SEQUENCE LISTING (78 sequences)

SEQ ID NO: 1 - RNA, 19 nt, Artificial
  RNA Oligomer directed to human cardiovascular gene
gcagcugcaugaaucccaa

SEQ ID NO: 2 - RNA, 19 nt, Artificial
  RNA Oligomer directed to human cardiovascular gene
aaagauuacuccaaagguu

SEQ ID NO: 3 - RNA, 19 nt, Artificial
  RNA Oligomer directed to human cardiovascular gene
ccaucgcccuguaugacua

SEQ ID NO: 4 - RNA, 19 nt, Artificial
  RNA Oligomer directed to human cardiovascular gene
guucagauaaauccacaug

SEQ ID NO: 5 - RNA, 19 nt, Artificial
  RNA Oligomer directed to human cardiovascular gene
cucacuagugggaagugua

SEQ ID NO: 6 - RNA, 19 nt, Artificial
  RNA Oligomer directed to human cardiovascular gene
ucagauaaauccacauggu

SEQ ID NO: 7 - RNA, 21 nt, Artificial
  RNA Oligomer directed to human cardiovascular gene
ggagguuaugacuuggaaagu

SEQ ID NO: 8 - RNA, 21 nt, Artificial
  RNA Oligomer directed to human cardiovascular gene
caagugccuagacuguuacuu

SEQ ID NO: 9 - RNA, 21 nt, Artificial
  RNA Oligomer directed to human cardiovascular gene
guacugucaucugucugucuu

SEQ ID NO: 10 - RNA, 21 nt, Artificial
  RNA Oligomer directed to human cardiovascular gene
guacugucguccgucugucuu

SEQ ID NO: 11 - RNA, 19 nt, Artificial
  RNA Oligomer directed to human cardiovascular gene
guucuaugggaaguaaugg

SEQ ID NO: 12 - RNA, 21 nt, Artificial
  RNA Oligomer directed to human cardiovascular gene
caagccuugucucaugaagaa

SEQ ID NO: 13 - RNA, 21 nt, Artificial
  RNA Oligomer directed to human cardiovascular gene
cgaaauugguccaacagaaga

SEQ ID NO: 14 - RNA, 19 nt, Artificial
  RNA Oligomer directed to human cardiovascular gene
ggaguccagaugaugacuu

SEQ ID NO: 15 - RNA, 19 nt, Artificial
  RNA Oligomer directed to human cardiovascular gene
gaguccagaugaugacuuu

SEQ ID NO: 16 - RNA, 19 nt, Artificial
  RNA Oligomer directed to human cardiovascular gene
cagaugaugacuuuugaca

SEQ ID NO: 17 - RNA, 19 nt, Artificial
  RNA Oligomer directed to human cardiovascular gene
ggauucgcuaccaucgcua

SEQ ID NO: 18 - RNA, 19 nt, Artificial
  RNA Oligomer directed to human cardiovascular gene
gaacuuuagucaccaugga

SEQ ID NO: 19 - RNA, 19 nt, Artificial
  RNA Oligomer directed to human cardiovascular gene
ggauccugcucuaugacau

SEQ ID NO: 20 - RNA, 21 nt, Artificial
  RNA Oligomer directed to human cardiovascular gene
gucucuucagagugucagcac

SEQ ID NO: 21 - RNA, 21 nt, Artificial
  RNA Oligomer directed to human cardiovascular gene
cuagguaaaacuuggaucaga

SEQ ID NO: 22 - RNA, 21 nt, Artificial
  RNA Oligomer directed to human cardiovascular gene
gcaacgagcucuucuacaucu

SEQ ID NO: 23 - RNA, 21 nt, Artificial
  RNA Oligomer directed to human cardiovascular gene
ccagcuauaucuaaagcuauu

SEQ ID NO: 24 - RNA, 21 nt, Artificial
  RNA Oligomer directed to human cardiovascular gene
cacuucaugugaaagacauuu

SEQ ID NO: 25 - RNA, 21 nt, Artificial
  RNA Oligomer directed to human cardiovascular gene
gaggaacacuggaucauguca

SEQ ID NO: 26 - RNA, 21 nt, Artificial
  RNA Oligomer directed to human cardiovascular gene
gcggaacauugguguaguugu

SEQ ID NO: 27 - RNA, 21 nt, Artificial
  RNA Oligomer directed to human cardiovascular gene
ccuccauugugcuuuagaaua

SEQ ID NO: 28 - RNA, 21 nt, Artificial
  RNA Oligomer directed to human cardiovascular gene
gaacaaggugguaucaucuuu

SEQ ID NO: 29 - RNA, 21 nt, Artificial
  RNA Oligomer directed to human cardiovascular gene
caaggaacuggcuaaauauga

SEQ ID NO: 30 - RNA, 19 nt, Artificial
  RNA Oligomer directed to human cardiovascular gene
ggacauagcauuguugggu

SEQ ID NO: 31 - RNA, 19 nt, Artificial
  RNA Oligomer directed to human cardiovascular gene
ggacacagcauuguugguu

SEQ ID NO: 32 - RNA, 21 nt, Artificial
  RNA Oligomer directed to human cardiovascular gene
ggaugacuaccgucaucauga

SEQ ID NO: 33 - RNA, 21 nt, Artificial
  RNA Oligomer directed to human cardiovascular gene
ccauaguuaucaccaaggaua

SEQ ID NO: 34 - RNA, 21 nt, Artificial
  RNA Oligomer directed to human cardiovascular gene
caguaggucagcucgauaucu

SEQ ID NO: 35 - RNA, 21 nt, Artificial
  RNA Oligomer directed to human cardiovascular gene
guaccaguguugagauuagcu

SEQ ID NO: 36 - RNA, 21 nt, Artificial
  RNA Oligomer directed to human cardiovascular gene
cgauggucaucguguaugacu

SEQ ID NO: 37 - RNA, 21 nt, Artificial
  RNA Oligomer directed to human cardiovascular gene
gaaguacacccuagugaguuu

SEQ ID NO: 38 - RNA, 21 nt, Artificial
  RNA Oligomer directed to human cardiovascular gene
gcacuuuaguguuagguaaaa

SEQ ID NO: 39 - RNA, 21 nt, Artificial
  RNA Oligomer directed to human cardiovascular gene
ccaagggggugcuuauguuua

SEQ ID NO: 40 - RNA, 19 nt, Artificial
  RNA Oligomer directed to human cardiovascular gene
uugggauucaugcagcugc

SEQ ID NO: 41 - RNA, 19 nt, Artificial
  RNA Oligomer directed to human cardiovascular gene
aaccuuuggaguaaucuuu

SEQ ID NO: 42 - RNA, 19 nt, Artificial
  RNA Oligomer directed to human cardiovascular gene
uagucauacagggcgaugg

SEQ ID NO: 43 - RNA, 19 nt, Artificial
  RNA Oligomer directed to human cardiovascular gene
cauguggauuuaucugaac

SEQ ID NO: 44 - RNA, 19 nt, Artificial
  RNA Oligomer directed to human cardiovascular gene
uacacuucccacuagugag

SEQ ID NO: 45 - RNA, 19 nt, Artificial
  RNA Oligomer directed to human cardiovascular gene
accauguggauuuaucuga

SEQ ID NO: 46 - RNA, 21 nt, Artificial
  RNA Oligomer directed to human cardiovascular gene
acuuuccaagucauaaccucc

SEQ ID NO: 47 - RNA, 21 nt, Artificial
  RNA Oligomer directed to human cardiovascular gene
aaguaacagucuaggcacuug

SEQ ID NO: 48 - RNA, 21 nt, Artificial
  RNA Oligomer directed to human cardiovascular gene
aagacagacagaugacaguac

SEQ ID NO: 49 - RNA, 21 nt, Artificial
  RNA Oligomer directed to human cardiovascular gene
aagacagacggacgacaguac

SEQ ID NO: 50 - RNA, 19 nt, Artificial
  RNA Oligomer directed to human cardiovascular gene
ccauuacuucccauagaac

SEQ ID NO: 51 - RNA, 21 nt, Artificial
  RNA Oligomer directed to human cardiovascular gene
uucuucaugagacaaggcuug

SEQ ID NO: 52 - RNA, 21 nt, Artificial
  RNA Oligomer directed to human cardiovascular gene
ucuucuguuggaccaauuucg

SEQ ID NO: 53 - RNA, 19 nt, Artificial
  RNA Oligomer directed to human cardiovascular gene
aagucaucaucuggacucc

SEQ ID NO: 54 - RNA, 19 nt, Artificial
  RNA Oligomer directed to human cardiovascular gene
aaagucaucaucuggacuc

SEQ ID NO: 55 - RNA, 19 nt, Artificial
  RNA Oligomer directed to human cardiovascular gene
ugucaaaagucaucaucug

SEQ ID NO: 56 - RNA, 19 nt, Artificial
  RNA Oligomer directed to human cardiovascular gene
uagcgaugguagcgaaucc

SEQ ID NO: 57 - RNA, 19 nt, Artificial
  RNA Oligomer directed to human cardiovascular gene
uccauggugacuaaaguuc

SEQ ID NO: 58 - RNA, 19 nt, Artificial
  RNA Oligomer directed to human cardiovascular gene
augucauagagcaggaucc

SEQ ID NO: 59 - RNA, 21 nt, Artificial
  RNA Oligomer directed to human cardiovascular gene
gugcugacacucugaagagac

SEQ ID NO: 60 - RNA, 21 nt, Artificial
  RNA Oligomer directed to human cardiovascular gene
ucugauccaaguuuuaccuag

SEQ ID NO: 61 - RNA, 21 nt, Artificial
  RNA Oligomer directed to human cardiovascular gene
agauguagaagagcucguugc

SEQ ID NO: 62 - RNA, 21 nt, Artificial
  RNA Oligomer directed to human cardiovascular gene
aauagcuuuagauauagcugg

SEQ ID NO: 63 - RNA, 21 nt, Artificial
  RNA Oligomer directed to human cardiovascular gene
aaaugucuuucacaugaagug

SEQ ID NO: 64 - RNA, 21 nt, Artificial
  RNA Oligomer directed to human cardiovascular gene
ugacaugauccaguguuccuc

SEQ ID NO: 65 - RNA, 21 nt, Artificial
  RNA Oligomer directed to human cardiovascular gene
acaacuacaccaauguuccgc

SEQ ID NO: 66 - RNA, 21 nt, Artificial
  RNA Oligomer directed to human cardiovascular gene
uauucuaaagcacaauggagg

SEQ ID NO: 67 - RNA, 21 nt, Artificial
  RNA Oligomer directed to human cardiovascular gene
aaagaugauaccaccuuguuc

SEQ ID NO: 68 - RNA, 21 nt, Artificial
  RNA Oligomer directed to human cardiovascular gene
ucauauuuagccaguuccuug

SEQ ID NO: 69 - RNA, 19 nt, Artificial
  RNA Oligomer directed to human cardiovascular gene
acccaacaaugcuaugucc

SEQ ID NO: 70 - RNA, 19 nt, Artificial
  RNA Oligomer directed to human cardiovascular gene
aaccaacaaugcugugucc

SEQ ID NO: 71 - RNA, 21 nt, Artificial
  RNA Oligomer directed to human cardiovascular gene
ucaugaugacgguagucaucc

SEQ ID NO: 72 - RNA, 21 nt, Artificial
  RNA Oligomer directed to human cardiovascular gene
uauccuuggugauaacuaugg

SEQ ID NO: 73 - RNA, 21 nt, Artificial
  RNA Oligomer directed to human cardiovascular gene
agauaucgagcugaccuacug

SEQ ID NO: 74 - RNA, 21 nt, Artificial
  RNA Oligomer directed to human cardiovascular gene
agcuaaucucaacacugguac

SEQ ID NO: 75 - RNA, 21 nt, Artificial
  RNA Oligomer directed to human cardiovascular gene
agucauacacgaugaccaucg

SEQ ID NO: 76 - RNA, 21 nt, Artificial
  RNA Oligomer directed to human cardiovascular gene
aaacucacuaggguguacuuc

SEQ ID NO: 77 - RNA, 21 nt, Artificial
  RNA Oligomer directed to human cardiovascular gene
uuuuaccuaacacuaaagugc

SEQ ID NO: 78 - RNA, 21 nt, Artificial
  RNA Oligomer directed to human cardiovascular gene
uaaacauaagcacccccuugg

(19) United States
(12) Patent Application Publication
(10) Pub. No.: US 2010/0035963 A1
(43) Pub. Date: Feb. 11, 2010

(54) Oligoribonucleotides and Methods of use Thereof for Treatment of Cardiovascular Disease

(76) Inventors: Ayelet Chajut, Ramat Hasharon (IL); Elhanan Pinner, Moshav Beit Yitzhak (IL)

Correspondence Address: COOPER & DUNHAM, LLP, 30 Rockefeller Plaza, 20th Floor, NEW YORK, NY, 10112, US

(21) Appl. No.: 11/991,881

(22) PCT Filed: Sep. 6, 2006
(86) PCT No.: PCT/IL2006/001036

§ 371 Date: Jun. 16, 2008

Related U.S. Application Data
(60) Provisional application No. 60/715,414, filed on Sep. 9, 2005.
(60) Provisional application No. 60/732,188, filed on Oct. 31, 2005.

(51) Int. Cl. A61K 31/713; C12N 15/113; C12N 15/85; A61P 9/00

(52) U.S. Cl. 514/44 A; 536/24.5; 435/320.1

(57) ABSTRACT

The invention relates to a double-stranded compound, preferably an oligoribonucleotide, which down-regulates the expression of one or more cardiovascular-related gene. The invention also relates to a pharmaceutical composition comprising the compound, or a vector capable of expressing the oligoribonucleotide compound, and a pharmaceutically acceptable carrier. The present invention also contemplates a method of treating a patient suffering from a cardiovascular disorder or other diseases comprising administering to the patient the pharmaceutical composition in a therapeutically effective dose so as to thereby treat the patient.

This application claims priority of U.S. Provisional patent applications No. 60/715,414, filed 9 Sep. 2005 and No. 60/732,188, filed 31 Oct. 2005, both of which are hereby incorporated by reference in their entirety. Throughout this application various patent and scientific publications are cited. The disclosures for these publications in their entireties are hereby incorporated by reference into this application to more fully describe the state of the art to which this invention pertains.

### BACKGROUND OF THE INVENTION

### siRNAs and RNA Interference

RNA interference (RNAi) is a phenomenon involving double-stranded (ds) RNA-dependent gene specific posttranscriptional silencing. Originally, attempts to study this phenomenon and to manipulate mammalian cells experimentally were frustrated by an active, non-specific antiviral defense mechanism which was activated in response to long dsRNA molecules; see Gil et al. 2000, Apoptosis, 5:107-114. Later it was discovered that synthetic duplexes of 21 nucleotide RNAs could mediate gene specific RNAi in mammalian cells, without the stimulation of the generic antiviral defense mechanisms (see Elbashir et al. Nature 2001, 411:494-498 and Caplen et al. Proc Natl Acad Sci 2001, 98:9742-9747). As a result, small interfering RNAs (siRNAs), which are short double-stranded RNAs, have become powerful tools in attempting to understand gene function.

Thus, RNA interference (RNAi) refers to the process of sequence-specific post-transcriptional gene silencing in mammals mediated by small interfering RNAs (siRNAs) (Fire et al, 1998, Nature 391, 806) or microRNAs (miRNAs) (Ambros V. Nature 431:7006, 350-355 (2004); and Bartel D P. Cell. 2004 Jan. 23; 116(2): 281-97 MicroRNAs: genomics, biogenesis, mechanism, and function). The corresponding process in plants is commonly referred to as specific post-transcriptional gene silencing or RNA silencing and is also referred to as quelling in fungi. An siRNA is a double-stranded RNA molecule which down-regulates or silences (prevents) the expression of a gene/mRNA of its endogenous (cellular) counterpart. RNA interference is based on the ability of dsRNA species to enter a specific protein complex, where it is then targeted to the complementary cellular RNA and specifically degrades it. Thus, the RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir et al 2001, Genes Dev., 15, 188). In more detail, longer dsRNAs are digested into short (17-29 bp) dsRNA fragments (also referred to as short inhibitory RNAs—“siRNAs”) by type III RNAses (DICER, DROSHA, etc., Bernstein et al., Nature, 2001, v.409, p. 363-6; Lee et al., Nature, 2003, 425, p. 415-9). The RISC protein complex recognizes these fragments and complementary mRNA. The whole process is culminated by endonuclease cleavage of target mRNA (McManus&Sharp, Nature Rev Genet, 2002, v.3, p. 737-47; Paddison &Hannon, Curr Opin Mol Ther. 2003 June; 5(3): 217-24). For information on these terms and proposed mechanisms, see Bernstein E., Denli A M. Hannon G J: 2001 The rest is silence. RNA. I; 7(11): 1509-21; Nishikura K.: 2001 A short primer on RNAi: RNA-directed RNA polymerase acts as a key catalyst. Cell. I 16; 107(4): 415-8 and PCT publication WO 01/36646 (Glover et al).

The selection and synthesis of siRNA corresponding to known genes has been widely reported; see for example Chalk A M, Wahlestedt C, Sonnhammer E L. 2004 Improved and automated prediction of effective siRNA Biochem. Biophys. Res. Commun. June 18; 319(1): 264-74; Sioud M, Leirdal M., 2004, Potential design rules and enzymatic synthesis of siRNAs, Methods Mol Biol.; 252:457-69; Levenkova N, Gu Q, Rux J J. 2004, Gene specific siRNA selector Bioinformatics. I 12; 20(3): 430-2. and Ui-Tei K, Naito Y, Takahashi F, Haraguchi T, Ohki-Hamazaki H, Juni A, Ueda R, Saigo K., Guidelines for the selection of highly effective siRNA sequences for mammalian and chick RNA interference Nucleic Acids Res. 2004 I 9; 32(3):936-48.Se also Liu Y, Braasch D A, Nulf C J, Corey D R. Efficient and isoform-selective inhibition of cellular gene expression by peptide nucleic acids, Biochemistry, 2004 I 24; 43(7):1921-7. See also PCT publications WO 2004/015107 (Atugen) and WO 02/44321 (Tuschl et al), and also Chiu Y L, Rana T M. siRNA function in RNAi: a chemical modification analysis, RNA 2003 September; 9(9):1034-48 and I U.S. Pat. Nos. 5,898,031 and 6,107,094 (Crooke) for production of modified/more stable siRNAs.

Several groups have described the development of DNA-based vectors capable of generating siRNA within cells. The method generally involves transcription of short hairpin RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

Several studies have revealed that siRNA therapeutics are effective in vivo in both mammals and in humans. Bitko et al., have shown that specific siRNA molecules directed against the respiratory syncytial virus (RSV) nucleocapsid N gene are effective in treating mice when administered intranasally (Bitko et al., “Inhibition of respiratory viruses by nasally administered siRNA”, Nat. Med. 2005, 11(1):50-55). A review of the use of siRNA in medicine was recently published by Barik S. in J. Mol. Med (2005) 83: 764-773). Furthermore, a phase I clinical study with short siRNA molecule that targets the VEGFR1 receptor for the treatment of Age-Related Macular Degeneration (AMD) has been conducted in human patients. The siRNA drug administered by an intravitreal inter-ocular injection was found effective and safe in 14 patients tested after a maximum of 157 days of follow up (Boston Globe Jan. 21, 2005).

### Myocardial Infarction

Myocardial infarction and related myocardial ischemia following coronary arteriosclerosis are the leading causes of hospital admissions in industrialized countries. Cardiovascular diseases continue to be the principle cause of death in the United States, Europe and Japan. The costs of the disease are high both in terms of morbidity and mortality, as well as in terms of the financial burden on health care systems.

Myocardial infarction generally occurs when there is an abrupt decrease in coronary blood flow following a thrombotic occlusion of a coronary artery previously damaged by atherosclerosis. The coronary artery diseases are often characterized by lesions or occlusions in the coronary arteries which may result in inadequate blood flow to the myocardium, or myocardial ischemia, which is typically responsible for such complications as angina pectoris, necrosis of cardiac tissue (myocardial infarction), and sudden death. In most cases, infarction occurs when an atherosclerotic plaque fissures, ruptures or ulcerates and when conditions favour thrombogenesis. In rare cases, infarction may be due to coronary artery occlusion caused by coronary emboli, congenital abnormalities, coronary spasm, and a wide variety of systemic, particularly inflammatory diseases. In individuals who have had a first MI, the risk of a repeat MI within the next year is 10-14%, despite maximal medical management including angioplasty and stent placement.

Myocardial infarction and related myocardial ischemia following coronary arteriosclerosis are currently treated by the use of drugs and by modifications in behavior and diet. In other cases, dilatation of coronary arteries may be achieved by such procedures as angioplasty, laser ablation, atherectomy, catheterization, and intravascular stents. For certain patients, coronary artery bypass grafting (CABG) is the preferred form of treatment to relieve symptoms and often increase life expectancy. CABG consists of direct anastomosis of a vessel segment to one or more of the coronary arteries.

In conclusion, currently there are no satisfactory modes of therapy for the prevention and/or treatment of cardiovascular-related diseases and there is a need therefore to develop novel compounds for this purpose. The novel compounds of this invention may also be used to treat other diseases and conditions described herein

### SUMMARY OF THE INVENTION

The invention provides novel double stranded oligoribonucleotides that inhibit the expression of specific genes that are up-regulated in cardiovascular-related diseases. The invention also provides a pharmaceutical composition comprising one or more such oligoribonucleotides, and a vector capable of expressing the oligoribonucleotide. The present invention also provides a method of treating a patient suffering from a cardiovascular-related disease comprising administering to the patient one or more oligoribonucleotides typically as a pharmaceutical composition, in a therapeutically effective dose so as to thereby treat the patient. The present invention also contemplates treating other disorders that are accompanied by an elevated expression of these genes.

In one aspect, the present invention provides novel double stranded oligoribonucleotides that inhibit the expression of the following specific genes: heparin-binding EGF-like growth factor (HB-EGF (DTR)b), spermidine/spermine N1-acetyltransferase (SSAT), steroid sensitive gene 1 (URB), transcript variant 1 (SSG1), IQ motif containing GPase activating protein 1 (IQ-GAP), sphingosine-1-phosphate phosphatase 1 (SGPP1), -serine palmitoyltransferase, long chain base subunit 2 (SPTLC2), Synaptopodin 2-like (SYNPO2L), ornithine decarboxylase 1 (ODC1), FXYD domain containing ion transport regulator 5 (FXYD5), pim-1 oncogene (PIM1).

### BRIEF DESCRIPTION OF THE FIGURES

FIG. 1 demonstrates the effect of specific siRNA compounds on the expression of endogenous heparin-binding EGF-like growth factor (HB-EGF (DTR)b) in primary cardiomyocytes.

FIG. 2 demonstrates the effect of specific siRNA compounds on the expression of endogenous Synaptopodin 2-like (SYNPO2L) in primary cardiomyocytes.

FIG. 3 demonstrates the effect of specific siRNA compounds on the expression of endogenous IQ motif containing GTPase activating protein 1 (IQ-GAP) in primary cardiomyocytes.

FIG. 4 demonstrates the effect of specific siRNA compounds on the expression of endogenous spermidine/spermine N1-acetyltransferase (SSAT) in primary cardiomyocytes.

FIG. 5 demonstrates the effect of specific siRNA compounds on the expression of endogenous steroid sensitive gene 1 (URB), transcript variant 1 (SSG1) in primary cardiomyocytes.

FIG. 6 demonstrates the effect of specific siRNA compounds on the expression of endogenous sphingosine-1-phosphate phosphatase (SGPP1) in primary cardiomyocytes.

FIG. 7 demonstrates the effect of specific siRNA compounds on the expression of endogenous serine palmitoyltransferase, long chain base subunit 2 (SPTLC2) in primary cardiomyocytes.

FIG. 8 demonstrates the effect of specific siRNA compounds on the expression of endogenous FXY domain containing ion transport regulator 5 (FXYD5) in primary cardiomyocytes.

FIG. 9 demonstrates the effect of specific siRNA compounds on the expression of endogenous corta (CTTN) in primary cardiomyocytes.

FIG. 10 demonstrates the effect of specific siRNA compounds on the expression of endogenous omit decarboxylase 1 (ODC1) in primary cardiomyocytes.

FIG. 11 demonstrates the effect of specific siRNA compounds on the expression of endogenous pim-oncogene (PIM1) in primary cardiomyocytes.

FIG. 12 demonstrates Synpo2L expression (using in situ hybridization) in Perivascular Cardiomyocytes of intact myocardium (A), in the periinfarct area at 24 hrs post-ligation (B) and at 24 days post-ligation (C).

### DETAILED DESCRIPTION OF THE INVENTION

The present invention relates generally to compounds which down-regulate expression of the cardiovascular-related genes according to the present invention particularly to novel small interfering RNAs (siRNAs), and to the use of these novel siRNAs in the treatment of various diseases and medical conditions in particular accompanied by an elevated level of the cardiovascular-related genes.

The present invention further relates to compounds which down-regulate expression of the cardiovascular-related genes listed in Table O and to the use of these compounds in the treatment of various pathologies, in particular pathologies related to cardiovascular diseases or medical conditions accompanied by an elevated level of the cardiovascular-related genes. Preferably, the present invention relates to compounds which down-regulate expression of the cardiovascular-related genes listed in Table N and to the use of these compounds in the treatment of various pathologies, in particular pathologies related to cardiovascular diseases or medical conditions accompanied by an elevated level of the cardiovascular-related genes. More preferably, the present invention relates to compounds which down-regulate expression of the cardiovascular-related genes listed in Table A and to the use of these compounds in the treatment of various pathologies, in particular pathologies related to cardiovascular diseases or medical conditions accompanied by an elevated level of the cardiovascular-related genes.

The invention further provides a use of a therapeutically effective dose of one or more compounds which down-regulate expression of the cardiovascular-related genes listed in Tables A, N or O, for the preparation of a composition for promoting recovery in a patient suffering from a cardiovascular disease or a disorder accompanied by an elevated level of the gene products of the genes listed in Tables A, N or O.

The present invention provides methods and compositions for inhibiting expression of specific target cardiovascular-related genes in vivo. In general, the method includes administering oligoribonucleotides, such as small interfering RNAs (i.e., siRNAs) that are targeted to one or more particular cardiovascular-related genes and hybridize to, or interact with, the mRNAs under biological conditions (within the cell), or a nucleic acid material that can produce siRNA in a cell, in an amount sufficient to down-regulate expression of the target gene by an RNA interference mechanism. In particular, the subject method can be used to inhibit expression of specific cardiovascular-related genes for treatment of a disease.

In accordance with the present invention, the siRNA molecules or inhibitors of the cardiovascular-related gene may be used as drugs to treat various pathologies in particular pathologies related to cardiovascular diseases or disorders accompanied by up-regulation of these genes.

The present invention provides double-stranded oligoribonucleotides (siRNAs), which down-regulate the expression of the cardiovascular-related genes of the present invention. An siRNA of the invention is a duplex oligoribonucleotide in which the sense strand is derived from the mRNA sequence of the selected gene, and the antisense strand is complementary to the sense strand. In general, some deviation from the target mRNA sequence is tolerated without compromising the siRNA activity (see e.g. Czauderna et al 2003 Nucleic Acids Research 31(11), 2705-2716). An siRNA of the invention inhibits gene expression on a post-transcriptional level with or without destroying the mRNA. Without being bound by theory, siRNA may target the mRNA for specific cleavage and degradation and/or may inhibit translation from the targeted message.

Table A below summarizes the details of selected 11 cardiovascular-related genes according to the present invention including their mouse and human reference numbers.

TABLE A

| | REFSEQ - | REFSEQ - |
|---|---|---|
| Gene/known function | mouse | human |
| HB-EGF (DTR)b - heparin-binding EGF-like growth factor. | NM_010415 | NM_001945 |
| Plays a role in cardiac hypertrophy and in stretch signaling. | | |
| SSAT - spermidine/spermine N1-acetyltransferase. | NM_009121 | NM_002970 |
| A rate-limiting enzyme in the catabolism of polyamines. | | |
| SSG1 - steroid sensitive gene 1 (URB), transcript variant 1. | NM_026439 | NM_199511 |
| Regulated by 17β-estradiol in the rat uterus and mammary gland in a tissue | | |
| specific manner. Androgen-regulated gene which is expressed in prostatic | | |
| smooth muscle cells. | | |
| IQ-GAP - IQ motif containing GTPase activating protein 1. | NM_016721 | NM_003870 |
| A homodimeric scaffolding protein (1657 AA) that binds to a diverse array of | | |
| signaling and structural molecules | | |
| SGPP1 - sphingosine-1-phosphate phosphatase 1. | NM_030750 | NM_030791 |
| A signaling lipid implicated in many biological processes among which are cell | | |
| growth and survival. | | |
| SPTLC2 - serine palmitoyltransferase, long chain base subunit 2. | NM_011479 | NM_004863 |
| An ER membrane protein which catalyzes the initial step of sphingolipid | | |
| biosynthesis: the condensation of palmitoyl coenzyme A (CoA) with L-serine to | | |
| generate 3-ketodihydrosphingosine. | | |
| SYNPO2L - Synaptopodin 2-like | NM_175132 | NM_024875 |
| An actin-binding protein which is found in the Z-disc of cardiac and skeletal | | |
| muscle cells. | | |
| ODC1 - ornithine decarboxylase 1. | NM_013614 | NM_002539 |
| A key regulatory enzyme in the biosynthesis of polyamines. | | |
| CTTN - cortactin. | NM_007803 | NM_005231 |
| Cortactin is a filamentous actin (F-actin)-binding protein that regulates | | |
| cytoskeketal networks that are necessary for endocytosis, cell migration and | | |
| invasion, adhesion, synaptic organisation and cell morphogenesis. | | |
| FXYD5 - FXYD domain containing ion transport regulator 5. | NM_008761 | NM_014164 |
| The FXYD gene family encodes a family of small membrane proteins that share | | |
| a 35-amino acid signature sequence domain, beginning with the sequence | | |
| PFXYD and containing 7 invariant and 6 highly conserved amino acids. | | |
| FXYD domain-containing proteins are tissue-specific regulators of the Na, K- | | |
| ATPase | | |
| PIM1 - pim-1 oncogene. | NM_008842 | NM_002648 |
| Involved in the control of cell growth, differentiation and apoptosis | | |

Generally, the siRNAs used in the present invention comprise a ribonucleic acid comprising a double stranded structure, whereby the double-stranded structure comprises a first strand and a second strand, whereby the first strand comprises a first stretch of contiguous nucleotides and whereby said first stretch is at least partially complementary to a target nucleic acid, and the second strand comprises a second stretch of contiguous nucleotides and whereby said second stretch is at least partially identical to a target nucleic acid. The strands may be modified on the sugar (see below for examples) and/or on the phosphate and/or on the base, or alternatively may be unmodified. In one aspect of the invention the said first strand and/or said second strand comprises a plurality of groups of modified nucleotides having a modification at the 2′-position whereby within the strand each group of modified nucleotides is flanked on one or both sides by a flanking group of nucleotides whereby the flanking nucleotides forming the flanking group of nucleotides is either an unmodified nucleotide or a nucleotide having a modification different from the modification of the modified nucleotides. Further, said first strand and/or said second strand may comprise said plurality of modified nucleotides and may comprises said plurality of groups of modified nucleotides.

The group of modified nucleotides and/or the group of flanking nucleotides may comprise a number of nucleotides whereby the number is selected from the group comprising one nucleotide to 10 nucleotides. In connection with any ranges specified herein it is to be understood that each range discloses any individual integer between the respective figures used to define the range including said two figures defining said range. In the present case the group thus comprises one nucleotide, two nucleotides, three nucleotides, four nucleotides, five nucleotides, six nucleotides, seven nucleotides, eight nucleotides, nine nucleotides and ten nucleotides.

The pattern of modified nucleotides of said first strand may be shifted by one or more nucleotides relative to the pattern of modified nucleotides of the second strand.

The modifications discussed above may be selected from the group comprising amino, fluoro, methoxy alkoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O—, S—, or N— alkyl; O—, S—, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP O 586520 B1 or EP O 618925 B1.

The double stranded structure of the siRNA may be blunt ended, on one or both sides. More specifically, the double stranded structure may be blunt ended on the double stranded structure's side which is defined by the 5′-end of the first strand and the 3′-end of the second strand, or the double stranded structure may be blunt ended on the double stranded structure's side which is defined by at the 3′-end of the first strand and the 5′-end of the second strand.

Additionally, at least one of the two strands may have an overhang of at least one nucleotide at the 5′-end; the overhang may consist of at least one deoxyribonucleotide. At least one of the strands may also optionally have an overhang of at least one nucleotide at the 3′-end.

The length of the double-stranded structure of the siRNA is typically from about 17 to 27 and more preferably 19 or 21 bases Further, the length of said first strand and/or the length of said second strand may independently from each other be selected from the group comprising the ranges of from about 15 to about 27 bases, 17 to 21 bases and 18 or 19 bases. A particular example is 27 bases.

Additionally, the complementarily between said first strand and the target nucleic acid may be perfect, or the duplex formed between the first strand and the target nucleic acid may comprise at least 15 nucleotides wherein there is one mismatch or two mismatches between said first strand and the target nucleic acid forming said double-stranded structure.

In some cases both the first strand and the second strand each comprise at least one group of modified nucleotides and at least one flanking group of nucleotides, whereby each group of modified nucleotides comprises at least one nucleotide and whereby each flanking group of nucleotides comprising at least one nucleotide with each group of modified nucleotides of the first strand being aligned with a flanking group of nucleotides on the second strand, whereby the most terminal 5′ nucleotide of the first strand is a nucleotide of the group of modified nucleotides, and the most terminal 3′ nucleotide of the second strand is a nucleotide of the flanking group of nucleotides. Each group of modified nucleotides may consist of a single nucleotide and/or each flanking group of nucleotides may consist of a single nucleotide.

Additionally, it is possible that on the first strand the nucleotide forming the flanking group of nucleotides is an unmodified nucleotide which is arranged in a 3′ direction relative to the nucleotide forming the group of modified nucleotides, and on the second strand the nucleotide forming the group of modified nucleotides is a modified nucleotide which is arranged in 5′ direction relative to the nucleotide forming the flanking group of nucleotides.

Further the first strand of the siRNA may comprise eight to twelve, preferably nine to eleven, groups of modified nucleotides, and the second strand may comprise seven to eleven, preferably eight to ten, groups of modified nucleotides.

The first strand and the second strand may be linked by a loop structure, which may be comprised of a non-nucleic acid polymer such as, inter alia, polyethylene glycol. Alternatively, the loop structure may be comprised of a nucleic acid.

Further, the 5′-terminus of the first strand of the siRNA may be linked to the 3′-terminus of the second strand, or the 3′-end of the first strand may be linked to the 5′-terminus of the second strand, said linkage being via a nucleic acid linker typically having a length between 10-2000 nucleobases.

In particular, the invention provides a compound having structure A:
5′ (N)ₓ-Z 3′ (antisense strand)
3′ Z′-(N′)_{y} 5′ (sense strand)
wherein each N and N′ is a ribonucleotide which may be modified or unmodified in its sugar residue and (N)ₓ and (N′)_{y} is oligomer in which each consecutive N or N′ is joined to the next N or N′ by a covalent bond;
wherein each of x and y is an integer between 19 and 40;
   wherein each of Z and Z′ may be present or absent, but if present is dTdT and is covalently attached at the 3′ terminus of the strand in which it is present;
and wherein the sequence of (N)ₓ comprises an antisense sequence to mRNA of a cardiovascular-related gene in particular to any of the cardiovascular-related genes listed in Table O and/or N, and preferably comprises one or more of the antisense sequences present in any of Tables B-M.

It will be readily understood by those skilled in the art that the compounds of the present invention consist of a plurality of nucleotides, which are linked through covalent linkages. Each such covalent linkage may be a phosphodiester linkage, a phosphothioate linkage, or a combination of both, along the length of the nucleotide sequence of the individual strand. Other possible backbone modifications are described inter alia in U.S. Pat. Nos. 5,587,361; 6,242,589; 6,277,967; 6,326,358; 5,399,676; 5,489,677; and 5,596,086.

In particular embodiments, x and y are preferably an integer between about 19 to about 27, most preferably from about 19 to about 25. In a particular embodiment of the compound of the invention, x may be equal to y (viz., x=y) and in preferred embodiments x=y=19 or x=y=21. In a particularly preferred embodiment x=y=19.

In one embodiment of the compound of the invention, Z and Z′ are both absent; in another embodiment one of Z or Z′ is present.

In one embodiment of the compound of the invention, all of the ribonucleotides of the compound are unmodified in their sugar residues.

In preferred embodiments of the compound of the invention, at least one ribonucleotide is modified in its sugar residue, preferably a modification at the 2′ position. The modification at the 2′ position results in the presence of a moiety which is preferably selected from the group comprising amino, fluoro, methoxy, alkoxy and alkyl groups. In a presently most preferred embodiment the moiety at the 2′ position is methoxy (2′-0-methyl).

In preferred embodiments of the invention, alternating ribonucleotides are modified in both the antisense and the sense strands of the compound. In particular the siRNA used in the Examples has been such modified such that a 2′ O-Me group was present on the first, third, fifth, seventh, ninth, eleventh, thirteenth, fifteenth, seventeenth and nineteenth nucleotide of the antisense strand, whereby the very same modification, i.e. a 2′-O-Me group was present at the second, fourth, sixth, eighth, tenth, twelfth, fourteenth, sixteenth and eighteenth nucleotide of the sense strand. Additionally, it is to be noted that the in case of these particular nucleic acids according to the present invention the first stretch is identical to the first strand and the second stretch is identical to the second strand and these nucleic acids are also blunt ended.

According to one preferred embodiment of the invention, the antisense and the sense strands of the siRNA molecule are both phosphorylated only at the 3′-terminus and not at the 5′-terminus. According to another preferred embodiment of the invention, the antisense and the sense strands are both non-phosphorylated both at the 3′-terminus and also at the 5′-terminus. According to yet another preferred embodiment of the invention, the 1^{st} nucleotide in the 5′ position in the sense strand is specifically modified to abolish any possibility of in vivo 5′-phosphorylation.

In another embodiment of the compound of the invention, the ribonucleotides at the 5′ and 3′ termini of the antisense strand are modified in their sugar residues, and the ribonucleotides at the 5′ and 3′ termini of the sense strand are unmodified in their sugar residues.

The invention further provides a vector capable of expressing any of the aforementioned oligoribonucleotides in unmodified form in a cell after which appropriate modification may be made.

The invention also provides a composition comprising one or more of the compounds of the invention in a carrier, preferably a pharmaceutically acceptable carrier. This composition may comprise a mixture of two or more different siRNAs.

The invention also provides a composition which comprises the above compound of the invention covalently or non-covalently bound to one or more compounds of the invention in an amount effective to inhibit the expression of the genes and a carrier. This composition may be processed intracellularly by endogenous cellular complexes to produce one or more oligoribonucleotides of the invention.

The invention also provides a composition comprising a carrier and one or more of the compounds of the invention in an amount effective to down-regulate expression in a cell of the genes of Table A, Table N and/or Table O, which compound comprises a sequence substantially complementary to the sequence of (N)ₓ.

Additionally the invention provides a method of down-regulating the expression of the genes of Table A, Table N and/or Table Oby at least 50% as compared to a control comprising contacting an mRNA transcript of one or more of the genes of Table A with one or more of the compounds of the invention.

In one embodiment the oligoribonucleotide is down-regulating the genes of Table A, Table N and/or Table O, whereby the down-regulation of the genes of Table A, Table N and/or Table O is selected from the group comprising down-regulation of gene function, down-regulation of gene polypeptide and down-regulation of gene mRNA expression.

In one embodiment the compound is down-regulating the encoded polypeptide, whereby the down-regulation of encoded polypeptide is selected from the group comprising down-regulation of polypeptide function (which may be examined by an enzymatic assay or a binding assay with a known interactor of the native gene/polypeptide, inter alia), down-regulation of protein (which may be examined by Western blotting, ELISA or immuno-precipitation, inter alia) and down-regulation of mRNA expression (which may be examined by Northern blotting, quantitative RT-PCR, in-situ hybridisation or microarray hybridisation, inter alia).

The invention also provides a method of treating a patient suffering from a cardiovascular disease or a disorder accompanied by an elevated level of the gene products of the genes listed in Table A, Table N and/or Table O comprising administering to the patient a composition of the invention in a therapeutically effective dose so as to thereby treat the patient.

The invention also provides a use of a therapeutically effective dose of one or more compounds of the invention for the preparation of a composition for promoting recovery in a patient suffering from a cardiovascular disease or a disorder accompanied by an elevated level of the gene products of the genes listed in Table A, Table N and/or Table O

The term “cardiovascular disease” or “cardiovascular disorder” includes myocardial ischemia-associated dysfunction, heart failure, coronary arteriosclerosis, coronary thrombosis, myocardial infarction, stroke, acute coronary syndrome, unstable angina, arrhythmia, cardiopulmonary arrest, coronary heart disease, valve disorders, cardiac chest pain and any other disease or disorder of the cardiovascular system.

More particularly, the invention provides an oligoribonucleotide wherein one strand comprises consecutive nucleotides having, from 5′ to 3′ one or more of the sequences set forth in Tables B-M (either sense strands or the antisense strands) or a homolog thereof, wherein in up to 2 of the nucleotides in each terminal region a base is altered.

The terminal region of the oligonucleotide refers to bases 1-4 and/or 16-19 in the 19-mer sequence and to bases 1-4 and/or 18-21 in the 21-mer sequence.

The presently most preferred compound of the invention is a blunt-ended 19-mer oligonucleotide, i.e. x=y=19 and Z and Z′ are both absent; wherein alternating ribonucleotides are modified at the 2′ position in both the antisense and the sense strands, wherein the moiety at the 2′ position is methoxy (2′-0-methyl) and wherein the ribonucleotides at the 5′ and 3′ termini of the antisense strand are modified in their sugar residues, and the ribonucleotides at the 5′ and 3′ termini of the sense strand are unmodified in their sugar residues.

In another aspect of the invention the oligonucleotide comprises a double-stranded structure, whereby such double-stranded structure comprises
a first strand and a second strand, whereby
the first strand comprises a first stretch of contiguous nucleotides and the second strand comprises a second stretch of contiguous nucleotides, whereby
the first stretch is either complementary or identical to a nucleic acid sequence coding for one of the genes listed in Table A, Table N and/or Table O and whereby the second stretch is either identical or complementary to a nucleic acid sequence coding for these genes.

In an embodiment the first stretch and/or the second stretch comprises from about 14 to 40 nucleotides, preferably about 18 to 30 nucleotides, more preferably from about 19 to 27 nucleotides and most preferably from about 19 to 25 nucleotides, in particular from about 19 to 21 nucleotides. In such an embodiment the oligonucleotide may be from 17-40 nucleotides in length.

Additionally, further nucleic acids according to the present invention comprise at least 14 contiguous nucleotides of any one of the polynucleotides in Tables B-M and more preferably 14 contiguous nucleotide base pairs at any end of the double-stranded structure comprised of the first stretch and second stretch as described above.

In an embodiment the first stretch comprises a sequence of at least 14 contiguous nucleotides of an oligonucleotide, whereby such oligonucleotide is selected from the group listed in Tables B-M

Additionally, further nucleic acids according to the present invention comprise at least 14 contiguous nucleotides of any one of the sequences listed in Tables B-M, and more preferably 14 contiguous nucleotide base pairs at any end of the double-stranded structure comprised of the first stretch and second stretch as described above. It will be understood by one skilled in the art that given the potential length of the nucleic acid according to the present invention and particularly of the individual stretches forming such nucleic acid according to the present invention, some shifts relative to the coding sequence to each side is possible, whereby such shifts can be up to 1, 2, 3, 4, 5 and 6 nucleotides in both directions, and whereby the thus generated double-stranded nucleic acid molecules shall also be within the present invention.

Delivery: Delivery systems aimed specifically at the enhanced and improved delivery of siRNA into mammalian cells have been developed, see, for example, Shen et al (FEBS letters 539: 111-114 (2003)), Xia et al., Nature Biotechnology 20: 1006-1010 (2002), Reich et al., Molecular Vision 9: 210-216 (2003), Sorensen et al. (J. Mol. Biol. 327: 761-766 (2003), Lewis et al., Nature Genetics 32: 107-108 (2002) and Simeoni et al., Nucleic Acids Research 31, 11: 2717-2724 (2003). siRNA has recently been successfully used for inhibition in primates; for further details see Tolentino et al., Retina 24(1) February 2004 I 132-138. Respiratory formulations for siRNA are described in U.S. patent application No. 2004/0063654 of Davis et al. Cholesterol-conjugated siRNAs (and other steroid and lipid conjugated siRNAs) can been used for delivery see Soutschek et al Nature 432: 173-177 (2004) Therapeutic silencing of an endogenous gene by systemic administration of modified siRNAs; and Lorenz et al. Bioorg. Med. Chemistry. Lett. 14:4975-4977 (2004) Steroid and lipid conjugates of siRNAs to enhance cellular uptake and gene silencing in liver cells.

The siRNAs or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art. The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic co-solvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art. In one specific embodiment of this invention topical and transdermal formulations are particularly preferred.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-4 weeks or longer. Treatment for many years or even lifetime treatment is also envisaged for some of the indications disclosed herein.

The term “treatment” as used herein refers to administration of a therapeutic substance effective to ameliorate symptoms associated with a disease, to lessen the severity or cure the disease, or to prevent the disease from occurring.

In a particular embodiment, the administration comprises intravenous administration. In another particular embodiment the administration comprises topical or local administration.

Another aspect of the invention is a method of treating a patient suffering from a cardiovascular disease, in particular for treating pathologies related to cardiovascular diseases or disorders accompanied by up-regulation of the cardiovascular-related genes according to the present invention, comprising administering to the patient a pharmaceutical composition of the invention in a therapeutically effective amount so as to thereby treat the patient.

Another aspect of the invention is a method of preventing a cardiovascular disease in a patient, comprising administering to the patient a pharmaceutical composition of the invention in a therapeutically effective amount so as to thereby treat the patient.

In another aspect of the invention a pharmaceutical composition is provided which comprises any of the above oligoribonucleotides listed in Tables B-M or vectors which express these oligonucleotides and a pharmaceutically acceptable carrier. Another aspect of the invention is the use of a therapeutically effective amount of any of the above oligoribonucleotides or vectors for the preparation of a medicament for promoting recovery in a patient suffering from a cardiovascular disease or a disorder which is accompanied by up-regulation of the cardiovascular-related genes according to the present invention.

The composition of the present invention may be used for the treatment of any cardiovascular disease or disorder such as a disorder related to heart failure or myocardial ischemia-associated dysfunction, for example a disorder caused by insufficient blood flow to the muscle tissue of the heart and the consequent ischemic damage. The decreased blood flow may be due to narrowing of the coronary arteries (coronary arteriosclerosis), or to obstruction by a thrombus (coronary thrombosis). The present composition is also effective when severe interruption of the blood supply to the myocardial tissue occurs resulting in necrosis of cardiac muscle (myocardial infarction). Other conditions that may be treated with the composition of the present invention are: stroke, acute coronary syndrome, unstable angina, arrhythmia, cardiopulmonary arrest, coronary heart disease, valve disorders, and cardiac chest pain.

The present invention also provides for a process of preparing a pharmaceutical composition, which comprises:
obtaining one or more double stranded compound of the invention; and
admixing said compound with a pharmaceutically acceptable carrier.

The present invention also provides for a process of preparing a pharmaceutical composition, which comprises admixing one or more compounds of the present invention with a pharmaceutically acceptable carrier.

In a preferred embodiment, the compound used in the preparation of a pharmaceutical composition is admixed with a carrier in a pharmaceutically effective dose. In a particular embodiment the compound of the present invention is conjugated to a steroid or to a lipid or to another suitable molecule e.g. to cholesterol.

Modifications or analogs of nucleotides can be introduced to improve the therapeutic properties of the nucleotides. Improved properties include increased nuclease resistance and/or increased ability to permeate cell membranes.

Accordingly, the present invention also includes all analogs of, or modifications to, a oligonucleotide of the invention that does not substantially affect the function of the polynucleotide or oligonucleotide. In a preferred embodiment such modification is related to the base moiety of the nucleotide, to the sugar moiety of the nucleotide and/or to the phosphate moiety of the nucleotide.

In embodiments of the invention, the nucleotides can be selected from naturally occurring or synthetically modified bases. Naturally occurring bases include adenine, guanine, cytosine, thymine and uracil. Modified bases of the oligonucleotides include inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thioalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structures of the nucleotides are fundamentally altered and are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone similar to that found in peptides. PNA analogs have been shown to be resistant to degradation by enzymes and to have extended lives in vivo and in vitro. Further, PNAs have been shown to bind more strongly to a complementary DNA sequence than to a DNA molecule. This observation is attributed to the lack of charge repulsion between the PNA strand and the DNA strand. Other modifications that can be made to oligonucleotides include polymer backbones, cyclic backbones, or acyclic backbones.

In one embodiment the modification is a modification of the phosphate moiety, whereby the modified phosphate moiety is selected from the group comprising phosphothioate.

The compounds of the present invention can be synthesized by any of the methods that are well-known in the art for synthesis of ribonucleic (or deoxyribonucleic) oligonucleotides. Such synthesis is, among others, described in Beaucage S. L. and Iyer R. P., Tetrahedron 1992; 48: 2223-2311, Beaucage S. L. and Iyer R. P., Tetrahedron 1993; 49: 6123-6194 and Caruthers M. H. et. al., Methods Enzymol. 1987; 154: 287-313, the synthesis of thiolates is, among others, described in Eckstein F., Annu. Rev. Biochem. 1985; 54: 367-402, the synthesis of RNA molecules is described in Sproat B., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

Other synthetic procedures are known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein.

Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via a tandem synthesis methodology, as described in US patent application publication No. US2004/0019001 (McSwiggen), wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

The compounds of the present invention can be delivered either directly or with viral or non-viral vectors. When delivered directly the sequences are generally rendered nuclease resistant Alternatively the sequences can be incorporated into expression cassettes or constructs such that the sequence is expressed in the cell as discussed herein below. Generally the construct contains the proper regulatory sequence or promoter to allow the sequence to be expressed in the targeted cell. Vectors optionally used for delivery of the compounds of the present invention are commercially available, and may be modified for the purpose of delivery of the compounds of the present invention by methods known to one of skill in the art.

It is also envisaged that a long oligonucleotide (typically 25-500 nucleotides in length) comprising one or more stem and loop structures, where stem regions comprise the sequences of the oligonucleotides of the invention, may be delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the invention. This oligonucleotide can be termed a tandem shRNA construct. It is envisaged that this long oligonucleotide is a single stranded oligonucleotide comprising one or more stem and loop structures, wherein each stem region comprises a sense and corresponding antisense siRNA sequence of the selected gene. In particular, it is envisaged that this oligonucleotide comprises sense and antisense siRNA sequences as depicted in Tables B-M.

As used herein, the term “polypeptide” refers to, in addition to a polypeptide, an oligopeptide, peptide and a full protein.

The compounds which down-regulate expression of the cardiovascular-related genes according to the present invention may be inter alia siRNA, antibodies, preferably neutralizing antibodies or fragments thereof, including single chain antibodies, antisense oligonucleotides, antisense DNA or RNA molecules, proteins, polypeptides and peptides including peptide-mimetics and dominant negatives, and also expression vectors expressing all the above. Additional inhibitors may be small chemical molecules, which generally have a molecular weight of less than 2000 daltons, more preferably less than 1000 daltons, even more preferably less than 500 daltons. These inhibitors may act as follows: small molecules may affect expression and/or activity; antibodies may affect activity; all kinds of antisense may affect the expression of the cardiovascular-related genes; and dominant negative polypeptides and peptidomimetics may affect activity; expression vectors may be used inter alia for delivery of antisense or dominant-negative polypeptides or antibodies.

The selection and synthesis of siRNA corresponding to the genes listed in Tables A, N or O may be conducted based on known methods; see for example Yi Pei and Thomas Tuschl 2006 On the art of identifying effective and specific siRNA. Nature Methods Vol 3 No 9 p-670-676; Chalk A M, Wahlestedt C, Sonnhammer E L. 2004 Improved and automated prediction of effective siRNA Biochem. Biophys. Res. Commun. June 18; 319(1): 264-74; Sioud M, Leirdal M., 2004, Potential design rules and enzymatic synthesis of siRNAs, Methods Mol Biol.; 252:457-69; Levenkova N, Gu Q, Rux J J. 2004, Gene specific siRNA selector Bioinformatics. I 12; 20(3): 430-2. and Ui-Tei K, Naito Y, Takahashi F, Haraguchi T, Ohki-Hamazaki H, Juni A, Ueda R, Saigo K., Guidelines for the selection of highly effective siRNA sequences for mammalian and chick RNA interference Nucleic Acids Res. 2004 I 9; 32(3):936-48.Se also Liu Y, Braasch D A, Nulf C J, Corey D R. Efficient and isoform-selective inhibition of cellular gene expression by peptide nucleic acids, Biochemistry, 2004 I 124; 43(7):1921-7.

### Antibody Production

By the term “antibody” as used in the present invention is meant both poly- and mono-clonal complete antibodies as well as fragments thereof, such as Fab, F(ab′)2, and Fv, which are capable of binding the epitopic determinant. These antibody fragments retain the ability to selectively bind with its antigen or receptor and are exemplified as follows, inter alia:
(1) Fab, the fragment which contains a monovalent antigen-binding fragment of an antibody molecule can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′2) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

Such fragments having antibody functional activity can be prepared by methods known to those skilled in the art (e.g. Bird et al. (1988) Science 242:423-426).

Conveniently, antibodies may be prepared against the immunogen or portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art, as described generally in Harlow and Lane (1988), Antibodies: A Laboratory Manual, Cold Spring Harbor Laboratory, Cold Spring Harbor, N.Y., and Borrebaeck (1992), Antibody Engineering—A Practical Guide, W.H. Freeman and Co., NY.

For producing polyclonal antibodies a host, such as a rabbit or goat, is immunized with the immunogen or immunogen fragment, generally with an adjuvant and, if necessary, coupled to a carrier, antibodies to the immunogen are collected from the sera. Further, the polyclonal antibody can be absorbed such that it is monospecific; that is, the sera can be absorbed against related immunogens so that no cross-reactive antibodies remain in the sera, rendering it monospecific.

For producing monoclonal antibodies the technique involves hyperimmunization of an appropriate donor with the immunogen, generally a mouse, and isolation of splenic antibody-producing cells. These cells are fused to an immortal cell, such as a myeloma cell, to provide a fused cell hybrid that is immortal and secretes the required antibody. The cells are then cultured, in bulk, and the monoclonal antibodies harvested from the culture media for use.

For producing recombinant antibody see generally Huston et al. (1991) “Protein engineering of single-chain Fv analogs and fusion proteins” in Methods in Enzymology (J J Langone, ed, Academic Press, New York, N.Y.) 203:46-88; Johnson and Bird (1991) “Construction of single-chain Fvb derivatives of monoclonal antibodies and their production in Escherichia coli in Methods in Enzymology (J J Langone, ed.; Academic Press, New York, N.Y.) 203:88-99; Mernaugh and Mernaugh (1995) “An overview of phage-displayed recombinant antibodies” in Molecular Methods In Plant Pathology (R P Singh and U S Singh, eds.; CRC Press Inc., Boca Raton, Fla.:359-365). In particular scFv antibodies are described in WO 2004/007553 (Tedesco and Marzari). Additionally, messenger RNAs from antibody-producing B-lymphocytes of animals, or hybridoma can be reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art (For a general discussion of conjugation of fluorescent or enzymatic moieties see Johnstone & Thorpe (1982.), Immunochemistry in Practice, Blackwell Scientific Publications, Oxford). The binding of antibodies to a solid support substrate is also well known in the art (for a general discussion, see Harlow & Lane (1988) Antibodies: A Laboratory Manual, Cold Spring Harbor Laboratory Publications, New York; and Borrebaeck (1992), Antibody Engineering—A Practical Guide, W.H. Freeman and Co.). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

One aspect of the invention comprises antibodies to the polypeptides/proteins expressed by the genes listed in Table O, in particular antibodies to the polypeptides/proteins expressed by the genes listed in Table N and most particularly antibodies to the polypeptides/proteins expressed by the genes listed in Table A, and a pharmaceutical composition comprising one or more such antibodies and a carrier. The present invention also provides a method of treating a patient suffering from a cardiovascular-related disease comprising administering to the patient one or more such antibodies typically as a pharmaceutical composition, in a therapeutically effective dose so as to thereby treat the patient.

### Screening of Inactivation Compounds:

Some of the compounds and compositions of the present invention may be used in a screening assay for identifying and isolating compounds that modulate the activity of the cardiovascular-related gene products, in particular compounds that modulate cardiovascular disorders or disorders accompanied by an elevated level of the cardiovascular-related polypeptide. The compounds to be screened comprise inter alia substances such as small chemical molecules and antisense oligonucleotides.

The inhibitory activity of the compounds of the present invention on the specific polypeptide activity or binding of the compounds of the present invention to the specific mRNA may be used to determine the interaction of an additional compound with the specific polypeptide, e.g., if the additional compound competes with the oligonucleotides of the present invention for expression inhibition, or if the additional compound rescues said inhibition. The inhibition or activation can be tested by various means, such as, inter alia, assaying for the product of the activity of the specific polypeptide or displacement of binding compound from the specific polypeptide in radioactive or fluorescent competition assays.

The present invention is illustrated in detail below with reference to Examples, but is not to be construed as being limited thereto.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

### General Methods in Molecular and Cell Biology

Standard molecular biology techniques known in the art and not specifically described were generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.) Methods of performing RT-PCR are also well known in the art.

### Primary Isolation of Neonatal Rat Cardiomyocytes (NRVM)

Sprague-Dawley or Wistar strains of rats can be used for isolation of neonatal cardiomyocytes.

The isolation process is most successful when the rats are 1-2-days-old.
1. Anaesthetize rats in box with chloroform.
2. Wash anaesthetized pups in ethanol 70%.
3. Dissect out the heart and place in a separate covered Petri dish with PBS, contained 1% of Pen-Strep and 8 U/ml of Heparin (5'000 U/ml stock solution) and glucose.
   Mix (100 ml PBS+1 ml Pen/Strep+200 μl 50% Glucose)
   Add 10 μl of Heparin from stock to 7 ml of (PBS+Pen/Strep+Glucose) mix.
4. Wash heart in PBS/Pen-Strep/Heparin and transfer to additional Petri dish with PBS/Pen-Strep/Glucose.
   Repeat dissection until the required number of hearts has been obtained.
   Hearts are kept at room temperature and should all be dissected within 30-40 minutes.

Mechanic dissociation. Using forceps, take each heart in turn from the PBS and place into upturned lid of the Petri dish.

Dissect out the ventricles with a sterile scalpel and retain in the dish.

Discard atria and associated vessels.

Use scalpel to mince ventricles finely until it is difficult to distinguish individual pieces of tissue.

Transfer fragments to 25-ml conical flask with magnetic stirring bar.

Enzymatic dissociation. 4×30 min incubations of minced tissue in flask with stirring bar in PBS/Glucose/Pen-Strep mix, that contains RDB enzyme (1: 50) at room temperature. (RDB is a plant proteolysis enzyme.

Centrifugation of upper phase from all steps at 1400 rpm, 5 min.

Pre-plating step. Re-suspend all pellets in F12/DMEN medium, plate cells on 15 cm non-coated dish for 40 min at 37° C.

Plating. Collect supernatant from the 15-cm dish and centrifuge again, count the cells and plate them on the collagen-coated dishes or fibronectin-coated stretchers.

Starvation. Before treatment, cells are starved for 24 hrs in DMEM-F12 medium containing pen/strep+glutamine without serum.

### Example 1

### Generation of Sequences for Active siRNA Compounds

Using proprietary algorithms and the known sequence of the target genes, the sequences of many potential siRNAs were generated. Table B below shows specific siRNAs to eleven cardiovascular genes; these siRNAs have been selected, chemically synthesized and tested for activity.

TABLE B

| | | | | |
|---|---|---|---|---|
| Oligo size | Index | Sense strand | Antisense strand | Species |

| | | | | |
|---|---|---|---|---|
| 19 | CTTN_3 | GCAGCUGCAUGAAUCCCAA | UUGGGAUUCAUGCAGCUGC | m, r, h |
| 19 | CTTN_2 | AAAGAUUACUCCAAAGGUU | AACCUUUGGAGUAAUCUUU | m, r |
| 19 | CTTN_1 | CCAUCGCCCUGUAUGACUA | UAGUCAUACAGGGCGAUGG | m, r |
| 19 | FXYD5_3 | GUUCAGAUAAAUCCACAUG | CAUGUGGAUUUAUCUGAAC | m, r |
| 19 | FXYD5_2 | CUCACUAGUGGGAAGUGUA | UACACUUCCCACUAGUGAG | m, r |
| 19 | FXYD5_1 | TCAGAUAAAUCCACAUGGU | ACCAUGUGGAUUUAUCUGA | m, r |
| 21 | HBEGF_4 | GGAGGUUAUGACUUGGAAAGU | ACUUUCCAAGUCAUAACCUCC | m, r |
| 21 | HBEGF_3 | CAAGUGCCUAGACUGUUACUU | AAGUAACAGUCUAGGCACUUG | m, r, h |
| 21 | HBEGF_2 | GUACUGUCAUCUGUCUGUCUU | AAGACAGACAGAUGACAGUAC | r |
| 21 | HBEGF_1 | GUACUGUCGUCCGUCUGUCUU | AAGACAGACGGACGACAGUAC | m |
| 19 | IQGAP1_3 | GUUCUAUGGGAAGUAAUGG | CCAUUACUUCCCAUAGAAC | m |
| 21 | IQGAP1_2 | CAAGCCUUGUCUCAUGAAGAA | UUCUUCAUGAGACAAGGCUUG | m, r |
| 21 | IQGAP1_1 | CGAAAUUGGUCCAACAGAAGA | UCUUCUGUUGGACCAAUUUCG | m, r |
| 19 | ODC1_3 | GGAGUCCAGAUGAUGACUU | AAGUCAUCAUCUGGACUCC | h, m, r |
| 19 | ODC1_2 | GAGUCCAGAUGAUGACUUU | AAAGUCAUCAUCUGGACUC | h, m, r |
| 19 | ODC1_1 | CAGAUGAUGACUUUUGACA | UGUCAAAAGUCAUCAUCUG | m, r |
| 19 | PIM1_4 | GGAUUCGCUACCAUCGCUA | UAGCGAUGGUAGCGAAUCC | m, r |
| 19 | PIM1_3 | GAACUUUAGUCACCAUGGA | UCCAUGGUGACUAAAGUUC | m, r |
| 19 | PIM1_2 | GGAUCCUGCUCUAUGACAU | AUGUCAUAGAGCAGGAUCC | m, r |
| 21 | PIM1_1 | GUCUCUUCAGAGUGUCAGCAC | GUGCUGACACUCUGAAGAGAC | m |
| 21 | SGPP1_3 | CUAGGUAAAACUUGGAUCAGA | UCUGAUCCAAGUUUUACCUAG | m |
| 21 | SGPP1_2 | GCAACGAGCUCUUCUACAUCU | AGAUGUAGAAGAGCUCGUUGC | m |
| 21 | SGPP1_1 | CCAGCUAUAUCUAAAGCUAUU | AAUAGCUUUAGAUAUAGCUGG | m |
| 21 | SPTLC2_3 | CACUUCAUGUGAAAGACAUUU | AAAUGUCUUUCACAUGAAGUG | m |
| 21 | SPTLC2_2 | GAGGAACACUGGAUCAUGUCA | UGACAUGAUCCAGUGUUCCUC | m |
| 21 | SPTLC2_1 | GOGGAACAUUGGUGUAGUUGU | ACAACUACACCAAUGUUCCGC | m |
| 21 | SSAT_5 | CCUCCAUUGUGCUUUAGAAUA | UAUUCUAAAGCACAAUGGAGG | r, m |
| 21 | SSAT_4 | GAACAAGGUGGUAUCAUCUUU | AAAGAUGAUACCACCUUGUUC | r |
| 21 | SSAT_3 | CAAGGAACUGGCUAAAUAUGA | UCAUAUUUAGCCAGUUCCUUG | r, m |
| 19 | SSAT_2 | GGACAUAGCAUUGUUGGGU | ACCCAACAAUGCUAUGUCC | m |
| 19 | SSAT_1 | GGACACAGCAUUGUUGGUU | AACCAACAAUGCUGUGUCC | r, h |
| 21 | SSG1_5 | GGAUGACUACCGUCAUCAUGA | UCAUGAUGACGGUAGUCAUCC | r, m, h |
| 21 | SSG1_4 | CCAUAGUUAUCACCAAGGAUA | UAUCCUUGGUGAUAACUAUGG | r, m |
| 21 | SSG1_3 | CAGUAGGUCAGCUCGAUAUCU | AGAUAUCGAGCUGACCUACUG | r |
| 21 | SSG1_2 | GUACCAGUGUUGAGAUUAGCU | AGCUAAUCUCAACACUGGUAC | r, m |
| 21 | SSG1_1 | CGAUGGUCAUCGUGUAUGACU | AGUCAUACACGAUGACCAUCG | r, m |
| 21 | SYNPO2L_3 | GAAGUACACCCUAGUGAGUUU | AAACUCACUAGGGUGUACUUC | m, r |
| 21 | SYNPO2L_2 | GCACUUUAGUGUUAGGUAAAA | UUUUACCUAACACUAAAGUGC | m |
| 21 | SYNPO2L_1 | CCAAGGGGGUGCUUAUGUUUA | UAAACAUAAGCACCCCCUUGG | m, r |

Table C below shows further siRNAs compounds specific for the CTTN gene that have been selected according to the present invention.

TABLE C

| No. | Source | Oligo Length | Sense siRNA | AntiSense siRNA | human 20357555 | human 20357551 | mouse 6681086 | rat 11177917 |
|---|---|---|---|---|---|---|---|---|
| FOR 19 | | | | | | | | |
| STRETCHES: | | | | | | | | |
| 1 | Cross Sp | 19 | GUCUGCUGUAGGCUUUGAA | UUCAAAGCCUACAGCAGAC | [576-594](19/19) | [576-594](19/19) | [532-550](19/19) | [402-420](19/19) |
| 2 | Cross Sp | 19 | GCAGCUGCAUGAAUCCCAA | UUGGGAUUCAUGCAGCUGC | [942-960](19/19) | [942-960](19/19) | [898-916](19/19) | [768-786](19/19) |
| 3 | Cross Sp | 19 | CUGCUGUAGGCUUUGAAUA | UAUUCAAAGCCUACAGCAG | [578-596](19/19) | [578-596](19/19) | [534-552](19/19) | [404-422](19/19) |
| 4 | Cross Sp | 19 | GGAAGUAUGGGGUGCAGAA | UUCUGCACCCCAUACUUCC | [986-1004](19/19) | [1097-1115](19/19) | [1053-1071](19/19) | [812-830](19/19) |
| 5 | Cross Sp | 19 | CUGCAUGAAUCCCAAAAAG | CUUUUUGGGAUUCAUGCAG | [946-964](19/19) | [946-964](19/19) | [902-920](19/19) | [772-790](19/19) |
| 6 | Cross Sp | 19 | GCUGCAUGAAUCCCAAAAA | UUUUUGGGAUUCAUGCAGC | [945-963](19/19) | [945-963](19/19) | [901-919](19/19) | [771-789](19/19) |
| 7 | Cross Sp | 19 | UCAGUCUGCUGUAGGCUUU | AAAGCCUACAGCAGACUGA | [573-591](19/19) | [573-591](19/19) | [529-547](19/19) | [399-417](19/19) |
| 8 | Cross Sp | 19 | GCUUUGAAUACCAGGGGAA | UUCCCCUGGUAUUCAAAGC | [587-605](19/19) | [587-605](19/19) | [543-561](19/19) | [413-431](19/19) |
| 9 | Cross Sp | 19 | CUGUAGGCUUUGAAUACCA | UGGUAUUCAAAGCCUACAG | [581-599](19/19) | [581-599](19/19) | [537-555](19/19) | [407-425](19/19) |
| 10 | Cross Sp | 19 | GUAUGGGGUGCAGAAGGAU | AUCCUUCUGCACCCCAUAC | [990-1008](19/19) | [1101-1119](19/19) | [1057-1075](19/19) | [816-834](19/19) |
| 11 | Cross Sp | 19 | AGUAUGGGGUGCAGAAGGA | UCCUUCUGCACCCCAUACU | [989-1007](19/19) | [1100-1118](19/19) | [1056-1074](19/19) | [815-833](19/19) |
| 12 | Cross Sp | 19 | GAAGUAUGGGGUGCAGAAG | CUUCUGCACCCCAUACUUC | [987-1005](19/19) | [1098-1116](19/19) | [1054-1072](19/19) | [813-831](19/19) |
| 13 | Cross Sp | 19 | UCUGCUGUAGGCUUUGAAU | AUUCAAAGCCUACAGCAGA | [577-595](19/19) | [577-595](19/19) | [533-551](19/19) | [403-421](19/19) |
| 14 | Cross Sp | 19 | UGCAUGAAUCCCAAAAAGA | UCUUUUUGGGAUUCAUGCA | [947-965](19/19) | [947-965](19/19) | [903-921](19/19) | [773-791](19/19) |
| 15 | Cross Sp | 19 | AGCUGCAUGAAUCCCAAAA | UUUUGGGAUUCAUGCAGCU | [944-962](19/19) | [944-962](19/19) | [900-918](19/19) | [770-788](19/19) |
| 16 | Cross Sp | 19 | UUGGUGUGCAGACAGACAG | CUGUCUGUCUGCACACCAA | [881-899](19/19) | [881-899](19/19) | [837-855](19/19) | [707-725](19/19) |
| 17 | Cross Sp | 19 | GCUUUGAGUAUCAAGGCAA | UUGCCUUGAUACUCAAAGC | [809-827](19/19) | [809-827](19/19) | [765-783](19/19) | [635-653](19/19) |
| 18 | Cross Sp | 19 | GGCUUUGAGUAUCAAGGCA | UGCCUUGAUACUCAAAGCC | [808-826](19/19) | [808-826](19/19) | [764-782](19/19) | [634-652](19/19) |
| 19 | Cross Sp | 19 | CCUCCCAGAAAGACUACUC | GAGUAGUCUUUCUGGGAGG | [620-638](19/19) | [620-638](19/19) | [576-594](19/19) | [446-464](19/19) |
| 20 | Cross Sp | 19 | GCCUCCCAGAAAGACUACU | AGUAGUCUUUCUGGGAGGC | [619-637](19/19) | [619-637](19/19) | [575-593](19/19) | [445-463](19/19) |
| 21 | Cross Sp | 19 | GGCUUUGAAUACCAGGGGA | UCCCCUGGUAUUCAAAGCC | [586-604](19/19) | [586-604](19/19) | [542-560](19/19) | [412-430](19/19) |
| 22 | Cross Sp | 19 | UGUAGGCUUUGAAUACCAG | CUGGUAUUCAAAGCCUACA | [582-600](19/19) | [582-600](19/19) | [538-556](19/19) | [408-426](19/19) |
| 23 | Cross Sp | 19 | GCUGUAGGCUUUGAAUACC | GGUAUUCAAAGCCUACAGC | [580-598](19/19) | [580-598](19/19) | [536-554](19/19) | [406-424](19/19) |
| 24 | Cross Sp | 19 | AGGAUUCGGCGGGAAGUAU | AUACUUCCCGCCGAAUCCU | [975-993](19/19) | [1086-1104](19/19) | [1042-1060](19/19) | [801-819](19/19) |
| 25 | Cross Sp | 19 | AAGGAUUCGGCGGGAAGUA | UACUUCCCGCCGAAUCCUU | [974-992](19/19) | [1085-1103](19/19) | [1041-1059](19/19) | [800-818](19/19) |
| 26 | Cross Sp | 19 | AAAGGAUUCGGCGGGAAGU | ACUUCCCGCCGAAUCCUUU | [973-991](19/19) | [1084-1102](19/19) | [1040-1058](19/19) | [799-817](19/19) |
| 27 | Cross Sp | 19 | AGUCUGCUGUAGGCUUUGA | UCAAAGCCUACAGCAGACU | [575-593](19/19) | [575-593](19/19) | [531-549](19/19) | [401-419](19/19) |
| 28 | Cross Sp | 19 | CAGCUGCAUGAAUCCCAAA | UUUGGGAUUCAUGCAGCUG | [943-961](19/19) | [943-961](19/19) | [899-917](19/19) | [769-787](19/19) |
| 29 | Cross Sp | 19 | UGCAGCUGCAUGAAUCCCA | UGGGAUUCAUGCAGCUGCA | [941-959](19/19) | [941-959](19/19) | [897-915](19/19) | [767-785](19/19) |
| 30 | Cross Sp | 19 | GUGUGCAGACAGACAGACA | UGUCUGUCUGUCUGCACAC | [884-902](19/19) | [884-902](19/19) | [840-858](19/19) | [710-728](19/19) |
| 31 | Cross Sp | 19 | UUGAAUACCAGGGGAAGAC | GUCUUCCCCUGGUAUUCAA | [590-608](19/19) | [590-608](19/19) | [546-564](19/19) | [416-434](19/19) |
| 32 | Cross Sp | 19 | GUAGGCUUUGAAUACCAGG | CCUGGUAUUCAAAGCCUAC | [583-601](19/19) | [583-601](19/19) | [539-557](19/19) | [409-427](19/19) |
| 33 | Cross Sp | 19 | GAUCAGUCUGCUGUAGGCU | AGCCUACAGCAGACUGAUC | [571-589](19/19) | [571-589](19/19) | [527-545](19/19) | [397-415](19/19) |
| 34 | Cross Sp | 19 | UGCUGUAGGCUUUGAAUAC | GUAUUCAAAGCCUACAGCA | [579-597](19/19) | [579-597](19/19) | [535-553](19/19) | [405-423](19/19) |
| 35 | Cross Sp | 19 | UGCAGAAGGAUCGGAUGGA | UCCAUCCGAUCCUUCUGCA | [998-1016](19/19) | [1109-1127](19/19) | [1065-1083](19/19) | [824-842](19/19) |
| 36 | Cross Sp | 19 | UAUGGGGUGCAGAAGGAUC | GAUCCUUCUGCACCCCAUA | [991-1009](19/19) | [1102-1120](19/19) | [1058-1076](19/19) | [817-835](19/19) |
| 37 | Cross Sp | 19 | AAGUAUGGGGUGCAGAAGG | CCUUCUGCACCCCAUACUU | [988-1006](19/19) | [1099-1117](19/19) | [1055-1073](19/19) | [814-832](19/19) |
| 38 | Cross Sp | 19 | UGGUGUGCAGACAGACAGA | UCUGUCUGUCUGCACACCA | [882-900](19/19) | [882-900](19/19) | [838-856](19/19) | [708-726](19/19) |
| 39 | Cross Sp | 19 | UUUGGUGUGCAGACAGACA | UGUCUGUCUGCACACCAAA | [880-898](19/19) | [880-898](19/19) | [836-854](19/19) | [706-724](19/19) |
| 40 | Cross Sp | 19 | CAGUCUGCUGUAGGCUUUG | CAAAGCCUACAGCAGACUG | [574-592](19/19) | [574-592](19/19) | [530-548](19/19) | [400-418](19/19) |
| 41 | Cross Sp | 19 | AGGCUUUGAAUACCAGGGG | CCCCUGGUAUUCAAAGCCUA | [585-603](19/19) | [585-603](19/19) | [541-559](19/19) | [411-429](19/19) |
| 42 | Cross Sp | 19 | UAGGCUUUGAAUACCAGGG | CCCUGGUAUUCAAAGCCUA | [584-602](19/19) | [584-602](19/19) | [540-558](19/19) | [410-428](19/19) |
| 43 | Cross Sp | 19 | AUCAGUCUGCUGUAGGCUU | AAGCCUACAGCAGACUGAU | [572-590](19/19) | [572-590](19/19) | [528-546](19/19) | [398-416](19/19) |
| 44 | Cross Sp | 19 | UUUGAAUACCAGGGGAAGA | UCUUCCCCUGGUAUUCAAA | [589-607](19/19) | [589-607](19/19) | [545-563](19/19) | [415-433](19/19) |
| 45 | Cross Sp | 19 | CUUUGAAUACCAGGGGAAG | CUUCCCCUGGUAUUCAAAG | [588-606](19/19) | [588-606](19/19) | [544-562](19/19) | [414-432](19/19) |
| 46 | Cross Sp | 19 | CCAACUAUGUGGAGCUGCG | CGCAGCUCCACAUAGUUGG | [1691-1709](19/19) | [1802-1820](19/19) | [1746-1764](19/19) | [1502-1520](19/19) |
| 47 | Cross Sp | 19 | GGGUGCAGAAGGAUCGGAU | AUCCGAUCCUUCUGCACCC | [995-1013](19/19) | [1106-1124](19/19) | [1062-1080](19/19) | [821-839](19/19) |
| 48 | Cross Sp | 19 | GGAUUCGGCGGGAAGUAUG | CAUACUUCCCGCCGAAUCC | [976-994](19/19) | [1087-1105](19/19) | [1043-1061](19/19) | [802-820](19/19) |
| 49 | Cross Sp | 19 | CCAAAGGAUUCGGCGGGAA | UUCCCGCCGAAUCCUUUGG | [971-989](19/19) | [1082-1100](19/19) | [1038-1056](19/19) | [797-815](19/19) |
| 50 | Cross Sp | 19 | ACUAUGUGGAGCUGCGGCA | UGCCGCAGCUCCACAUAGU | [1694-1712](19/19) | [1805-1823](19/19) | [1749-1767](19/19) | [1505-1523](19/19) |
| 51 | Cross Sp | 19 | GGUGUGCAGACAGACAGAC | GUCUGUCUGUCUGCACACC | [883-901](19/19) | [883-901](19/19) | [839-857](19/19) | [709-727](19/19) |
| 52 | Cross Sp | 19 | ACCAGGGCAAGACGGAGAA | UUCUCCGUCUUGCCCUGGU | [707-725](19/19) | [707-725](19/19) | [663-681](19/19) | [533-551](19/19) |
| 53 | Cross Sp | 19 | AUGUGGAGCUGCGGCAGUA | UACUGCCGCAGCUCCACAU | [1697-1715](19/19) | [1808-1826](19/19) | [1752-1770](19/19) | [1508-1526](19/19) |
| 54 | Cross Sp | 19 | CAACUAUGUGGAGCUGCGG | CCGCAGCUCCACAUAGUUG | [1692-1710](19/19) | [1803-1821](19/19) | [1747-1765](19/19) | [1503-1521](19/19) |
| 55 | Cross Sp | 19 | GGUGCAGAAGGAUCGGAUG | CAUCCGAUCCUUCUGCACC | [996-1014](19/19) | [1107-1125](19/19) | [1063-1081](19/19) | [822-840](19/19) |
| 56 | Cross Sp | 19 | AUGGGGUGCAGAAGGAUCG | CGAUCCUUCUGCACCCCAU | [992-1010](19/19) | [1103-1121](19/19) | [1059-1077](19/19) | [818-836](19/19) |
| 57 | Cross Sp | 19 | GGGAAGUAUGGGGUGCAGA | UCUGCACCCCAUACUUCCC | [985-1003](19/19) | [1096-1114](19/19) | [1052-1070](19/19) | [811-829](19/19) |
| 58 | Cross Sp | 19 | GAUUCGGCGGGAAGUAUGG | CCAUACUUCCCGCCGAAUC | [977-995](19/19) | [1088-1106](19/19) | [1044-1062](19/19) | [803-821](19/19) |
| 59 | Cross Sp | 19 | CAAAGGAUUCGGCGGGAAG | CUUCCCGCCGAAUCCUUUG | [972-990](19/19) | [1083-1101](19/19) | [1039-1057](19/19) | [798-816](19/19) |
| 60 | Cross Sp | 19 | AGGGCAAGACGGAGAAGCA | UGCUUCUCCGUCUUGCCCU | [710-728](19/19) | [710-728](19/19) | [666-684](19/19) | [536-554](19/19) |
| 61 | Cross Sp | 19 | UACCAGGGCAAGACGGAGA | UCUCCGUCUUGCCCUGGUA | [706-724](19/19) | [706-724](19/19) | [662-680](19/19) | [532-550](19/19) |
| 62 | Cross Sp | 19 | ACUACCAGGGCAAGACGGA | UCCGUCUUGCCCUGGUAGU | [704-722](19/19) | [704-722](19/19) | [660-678](19/19) | [530-548](19/19) |
| 63 | Cross Sp | 19 | UAUGUGGAGCUGCGGCAGU | ACUGCCGCAGCUCCACAUA | [1696-1714](19/19) | [1807-1825](19/19) | [1751-1769](19/19) | [1507-1525](19/19) |
| 64 | Cross Sp | 19 | GUGCAGAAGGAUCGGAUGG | CCAUCCGAUCCUUCUGCAC | [997-1015](19/19) | [1108-1126](19/19) | [1064-1082](19/19) | [823-841](19/19) |
| 65 | Cross Sp | 19 | AACUAUGUGGAGCUGCGGC | GCCGCAGCUCCACAUAGUU | [1693-1711](19/19) | [1804-1822](19/19) | [1748-1766](19/19) | [1504-1522](19/19) |
| 66 | Cross Sp | 19 | GCCAACUAUGUGGAGCUGC | GCAGCUCCACAUAGUUGGC | [1690-1708](19/19) | [1801-1819](19/19) | [1745-1763](19/19) | [1501-1519](19/19) |
| 67 | Cross Sp | 19 | AUUCGGCGGGAAGUAUGGG | CCCAUACUUCCCGCCGAAU | [978-996](19/19) | [1089-1107](19/19) | [1045-1063](19/19) | [804-822](19/19) |
| 68 | Human | 19 | CAAGACUACUCCAAAGGAU | AUCCUUUGGAGUAGUCUUG | [962-979](18/18) | [1072-1090](19/19) | [696-711](15/16) | [566-581](15/16) |
| 69 | Human | 19 | GCAACUUAUUGUAUCUGAA | UUCAGAUACAAUAAGUUGC | [3041-3059](19/19) | [3151-3169](19/19) | — | — |
| 70 | Human | 19 | GAACCCUCCUCCUGUCAAU | AUUGACAGGAGGAGGGUUC | [2656-2674](19/19) | [2766-2784](19/19) | — | — |
| 71 | Human | 19 | CUAGCAGAGACCCUGGUUU | AAACCAGGGUCUCUGCUAG | [2306-2324](19/19) | [2416-2434](19/19) | — | — |
| 72 | Human | 19 | GGAGAAGGAACUUGAAACA | UGUUUCAAGUUCCUUCUCC | [384-402](19/19) | [384-402](19/19) | — | — |
| 73 | Human | 19 | GGAAUAUACACAUUGCUUU | AAAGCAAUGUGUAUAUUCC | [1846-1864](19/19) | [1956-1974](19/19) | — | — |
| 74 | Human | 19 | CUACAGAUCAGGCCUUCUU | AAGAAGGCCUGAUCUGUAG | [1759-1777](19/19) | [1870-1888](19/19) | — | — |
| 76 | Human | 19 | GAAGCUGUGACCAGCAAAA | UUUUGCUGGUCACAGCUUC | [1084-1102](19/19) | [1195-1213](19/19) | — | [913-928](16/16) |
| 77 | Human | 19 | CAACCUUUGAGGAUGUCAC | GUGACAUCCUCAAAGGUUG | [1028-1046](19/19) | [1139-1157](19/19) | — | — |
| 78 | Human | 19 | GGAUCGGAUGGAUAAGAAU | AUUCUUAUCCAUCCGAUCC | [1005-1023](19/19) | [1116-1134](19/19) | [1072-1090](18/19) | [831-848](17/18) |
| 79 | Human | 19 | GCAGAAGGAUCGGAUGGAU | AUCCAUCCGAUCCUUCUGC | [999-1017](19/19) | [1110-1128](19/19) | [1066-1083](18/18) | [825-842](18/18) |
| 80 | Human | 19 | GCAUGAAUCCCAAAAAGAU | AUCUUUUUGGGAUUCAUGC | [948-965](18/18) | [948-966](19/19) | [904-921](18/18) | [774-791](18/18) |
| 81 | Human | 19 | CUGAUUUUGUGAAUGAUGU | ACAUCAUUCACAAAAUCAG | [251-269](19/19) | [251-269](19/19) | [207-225](19/19) | [77-95](18/19) |
| 82 | Human | 19 | GGGAUCACCAGGAGAAAUU | AAUUUCUCCUGGUGAUCCC | [923-941](19/19) | [923-941](19/19) | — | — |
| 83 | Human | 19 | AGACAGACAAGACAAAUGU | ACAUUUGUCUUGUCUGUCU | [849-912](19/19) | [894-912](19/19) | — | — |
| 84 | Human | 19 | GACAGACAGACAAGACAAA | UUUGUCUUGUCUGUCUGUC | [891-909](19/19) | [891-909](19/19) | [847-864](17/18) | [717-734](17/18) |
| 85 | Human | 19 | CAGACAGACAGACAAGACA | UGUCUUGUCUGUCUGUCUG | [889-907](19/19) | [889-907](19/19) | [845-863](18/19) | [715-733](18/19) |
| 86 | Human | 19 | CGACCCUGAUUUUGUGAAU | AUUCACAAAAUCAGGGUCG | [246-264](19/19) | [246-264](19/19) | [206-220](15/15) | [73-89](17/17) |
| 87 | Human | 19 | GAAAGGGUUUGGAGGAAAA | UUUUCCUCCAAACCCUUUC | [861-879](19/19) | [861-879](19/19) | [818-834](16/17) | [688-704](16/17) |
| 88 | Human | 19 | CCCAGAAAGACUAUGUGAA | UUCACAUAGUCUUUCUGGG | [845-863](19/19) | [845-863](19/19) | [801-816](16/16) | [671-686](16/16) |
| 89 | Human | 19 | GGAGACCGACCCUGAUUUU | AAAAUCAGGGUCGGUCUCC | [240-258](19/19) | [240-258](19/19) | — | [66-84](18/19) |
| 90 | Human | 19 | GCAAAACGGAGAAGCACGA | UCGUGCUUCUCCGUUUUGC | [824-842](19/19) | [824-842](19/19) | [669-684](15/16) | [539-557](18/19) |
| 91 | Human | 19 | GUAUCGACAAGGACAAAGU | ACUUUGUCCUUGUCGAUAC | [773-791](19/19) | [773-791](19/19) | [734-744](11/11) | [604-614](11/11) |
| 92 | Human | 19 | AGAGAUUACUCCAAAGGUU | AACCUUUGGAGUAAUCUCU | [739-757](19/19) | [739-757](19/19) | [697-713](17/17) | [567-583](17/17) |
| 93 | Human | 19 | GCACGAGUCACAGAGAGAU | AUCUCUCUGUGACUCGUGC | [726-744](19/19) | [726-744](19/19) | — | [552-570](18/19) |
| 94 | Human | 19 | CUACUCCAGUGGUUUUGGC | GCCAAAACCACUGGAGUAG | [633-651](19/19) | [633-651](19/19) | — | [459-473](15/15) |
| 95 | Human | 19 | GGACAGAGUUGAUCAGUCU | AGACUGAUCAACUCUGUCC | [561-579](19/19) | [561-579](19/19) | — | — |
| 96 | Human | 19 | CAAGUUUGGUGUCCAGAUG | CAUCUGGACACCAAACUUG | [543-561](19/19) | [543-561](19/19) | [499-517](18/19) | [369-387](18/19) |
| 97 | Human | 19 | GAAUAUCAGUCGAAACUUU | AAAGUUUCGACUGAUAUUC | [481-499](19/19) | [481-491](19/19) | — | — |
| 98 | Human | 19 | CCACGAAUAUCAGUCGAAA | UUUCGACUGAUAUUCGUGG | [477-495](19/19) | [477-495](19/19) | — | — |
| 99 | Human | 19 | ACACCUCAUAGGUAUGAUU | AAUCAUACCUAUGAGGUGU | [3070-3088](19/19) | [3180-3198](19/19) | — | — |
| 100 | Human | 19 | CUGUCGGCCACGAAUAUCA | UGAUAUUCGUGGCCGACAG | [470-488](19/19) | [470-488](19/19) | — | — |
| 101 | Human | 19 | GCAAUGAGGUCGGGUUUUA | UAAAACCCGACCUCAUUGC | [3019-3037](19/19) | [3129-3147](19/19) | — | — |
| 102 | Human | 19 | CCUUGCUUUACCACAAUGA | UCAUUGUGGUAAAGCAAGG | [3000-3018](19/19) | [3110-3128](19/19) | — | — |
| 103 | Human | 19 | CAUCAUCCUUGCUUUACCA | UGGUAAAGCAAGGAUGAUG | [2994-3012](19/19) | [3104-3122](19/19) | — | — |
| 104 | Human | 19 | CUCCUGAGGUGCAUUUUCU | AGAAAAUGCACCUCAGGAG | [2975-2993](19/19) | [3085-3103](19/19) | — | — |
| 105 | Human | 19 | CGUGAAGCGUGUUUUUGCU | AGCAAAAACACGCUUCACG | [2958-2976](19/19) | [3068-3086](19/19) | — | — |
| 106 | Human | 19 | CAGAAGGUCACGUGGAAAU | AUUUCCACGUGACCUUCUG | [2912-2930](19/19) | [3022-3040](19/19) | — | — |
| 107 | Human | 19 | GGAAACUCAUCUCCUUCCU | AGGAAGGAGAUGAGUUUCC | [2835-2853](19/19) | [2945-2963](19/19) | — | — |
| 108 | Human | 19 | GUAGGAAACUCAUCUCCUU | AAGGAGAUGAGUUUCCUAC | [2832-2850](19/19) | [2942-2960](19/19) | — | — |
| 109 | Human | 19 | UCACUUUGUAGGAAACUCA | UGAGUUUCCUACAAAGUGA | [2825-2843](19/19) | [2935-2953](19/19) | — | — |
| 110 | Human | 19 | CCACUCACUUUGUAGGAAA | UUUCCUACAAAGUGAGUGG | [2821-2839](19/19) | [2931-2949](19/19) | — | — |
| 111 | Human | 19 | GAACAAGACCGAAUGGAUA | UAUCCAUUCGGUCUUGUUC | [445-463](19/19) | [445-463](19/19) | — | — |
| 112 | Human | 19 | GGAACAAGACCGAAUGGAU | AUCCAUUCGGUCUUGUUCC | [444-462](19/19) | [444-462](19/19) | — | — |
| 113 | Human | 19 | GCAGAUGAGUGUGUGUUCU | AGAACACACACUCAUCUGC | [2751-2769](19/19) | [2861-2879](19/19) | — | — |
| 114 | Human | 19 | GCCAAAAUAUCAUGUUCAA | UUGAACAUGAUAUUUUGGC | [2692-2710](19/19) | [2802-2820](19/19) | — | — |
| 115 | Human | 19 | CAAUGGGGGUGUAGUAUUU | AAAUACUACACCCCCAUUG | [2671-2689](19/19) | [2781-2799](19/19) | — | — |
| 116 | Human | 19 | AAAGGCUGAUGUCUUAACU | AGUUAAGACAUCAGCCUUU | [2520-2538](19/19) | [2630-2648](19/19) | — | — |
| 117 | Human | 19 | CACAAAGGCUGAUGUCUUA | UAAGACAUCAGCCUUUGUG | [2517-2535](19/19) | [2627-2645](19/19) | — | — |
| 118 | Human | 19 | CCAUGGCUAUGGAGGGAAA | UUUCCCUCCAUAGCCAUGG | [417-435](19/19) | [417-435](19/19) | — | [243-260](17/18) |
| 119 | Human | 19 | GUCACUGAGUCCUUGAAAU | AUUUCAAGGACUCAGUGAC | [2427-2445](19/19) | [2537-2555](19/19) | — | — |
| 120 | Human | 19 | GUGUCACUGAGUCCUUGAA | UUCAAGGACUCAGUGACAC | [2425-2443](19/19) | [2535-2553](19/19) | — | — |
| 121 | Human | 19 | CCAAAAGCUUCCCAUGGCU | AGCCAUGGGAAGCUUUUGG | [406-424](19/19) | [406-424](19/19) | — | [238-250](13/13) |
| 122 | Human | 19 | CAGAGACCCUGGUUUUUUU | AAAAAAACCAGGGUCUCUG | [2310-2328](19/19) | [2420-2438](19/19) | — | — |
| 123 | Human | 19 | UGAAACAGGACCAAAAGCU | AGCUUUUGGUCCUGUUUCA | [396-414](19/19) | [396-414](19/19) | — | — |
| 124 | Human | 19 | GGAAAUCUGCCUAUGUCAU | AUGACAUAGGCAGAUUUCC | [2257-2275](19/19) | [2367-2385](19/19) | — | — |
| 125 | Human | 19 | GAAGGAACUUGAAACAGGA | UCCUGUUUCAAGUUCCUUC | [387-405](19/19) | [387-405](19/19) | — | — |
| 126 | Human | 19 | GGAGCUCGCAUUCUCUUGU | ACAAGAGAAUGCGAGCUCC | [2026-2044](19/19) | [2136-2154](19/19) | — | — |
| 127 | Human | 19 | GGACUUUGGUAAUUGGUUU | AAACCAAUUACCAAAGUCC | [1947-1965](19/19) | [2057-2075](19/19) | — | — |
| 128 | Human | 19 | GGAGGACUUUGGUAAUUGG | CCAAUUACCAAAGUCCUCC | [1944-1962](19/19) | [2054-2072](19/19) | — | — |
| 129 | Human | 19 | CCACAGAAUUUGCUAAUAU | AUAUUAGCAAAUUCUGUGG | [1906-1924](19/19) | [2016-2034](19/19) | — | — |
| 130 | Human | 19 | GGGGAAUAUACACAUUGCU | AGCAAUGUGUAUAUUCCCC | [1844-1862](19/19) | [1954-1972](19/19) | — | — |
| 131 | Human | 19 | GGAGGGGAAUAUACACAUU | AAUGUGUAUAUUCCCCUCC | [1841-1859](19/19) | [1951-1969](19/19) | — | — |
| 132 | Human | 19 | GGGAGAAUGUCUUUCAAGA | UCUUGAAAGACAUUCUCCC | [350-368](19/19) | [350-368](19/19) | [308-324](16/17) | [178-194](16/17) |
| 133 | Human | 19 | GAGGGAGAAUGUCUUUCAA | UUGAAAGACAUUCUCCCUC | [348-366](19/19) | [348-366](19/19) | [308-318](11/11) | [178-188](11/11) |
| 134 | Human | 19 | CCUGGAUCCUCACACUACA | UGUAGUGUGAGGAUCCAGG | [1745-1763](19/19) | [1856-1874](19/19) | — | — |
| 135 | Human | 19 | GCUCUUCCCAGCCAACUAU | AUAGUUGGCUGGGAAGAGC | [1680-1698](19/19) | [1791-1809](19/19) | [1735-1753](19/19) | — |
| 136 | Human | 19 | UCACCAACAUCGAGAUGAU | AUCAUCUCGAUGUUGGUGA | [1619-1637](19/19) | [1730-1748](19/19) | — | — |
| 137 | Human | 19 | GCGAUGAUGAGAUCUCAUU | AAUGAGAUCUCAUCAUCGC | [1583-1601](19/19) | [1694-1712](19/19) | [1638-1653](16/16) | — |
| 138 | Human | 19 | CCAGGAGCAUAUCAACAUA | UAUGUUGAUAUGCUCCUGG | [321-339](19/19) | [321-339](19/19) | — | — |
| 139 | Human | 19 | ACAGCACCUACGAUGAGUA | UACUCAUCGUAGGUGCUGU | [1511-1529](19/19) | [1622-1640](19/19) | [1570-1580](11/11) | [1326-1336](11/11) |
| 140 | Human | 19 | GGCACCAGGAGCAUAUCAA | UUGAUAUGCUCCUGGUGCC | [317-335](19/19) | [317-335](19/19) | — | — |
| 141 | Human | 19 | CACAGAGGCUGUCUAUGAA | UUCAUAGACAGCCUCUGUG | [1458-1476](19/19) | [1569-1587](19/19) | — | — |
| 142 | Human | 19 | UCAGAGCUAACUUUGAAAA | UUUUCAAAGUUAGCUCUGA | [1112-1130](19/19) | [1223-1241](19/19) | [1184-1197](14/14) | [943-955](13/13) |
| 143 | Human | 19 | UAACAUCAGAGCUAACUUU | AAAGUUAGCUCUGAUGUUA | [1107-1125](19/19) | [1218-1236](19/19) | — | — |
| 144 | Human | 19 | CAAGUAACAUCAGAGCUAA | UUAGCUCUGAUGUUACUUG | [1103-1121](19/19) | [1214-1232](19/19) | — | — |
| 145 | Human | 19 | GCUGUGACCAGCAAAACAA | UUGUUUUGCUGGUCACAGC | [1087-1105](19/19) | [1198-1216](19/19) | [1160-1170](11/11) | [913-929](17/17) |
| 146 | Human | 19 | GAAGACAGUACCUGUCGAA | UUCGACAGGUACUGUCUUC | [1068-1086](19/19) | [1179-1197](19/19) | — | — |
| 147 | Human | 19 | CCUACCAGAAGACAGUACC | GGUACUGUCUUCUGGUAGG | [1061-1079](19/19) | [1172-1190](19/19) | — | — |
| 148 | Human | 19 | CUGCCUACCAGAAGACAGU | ACUGUCUUCUGGUAGGCAG | [1058-1076](19/19) | [1169-1187](19/19) | — | — |
| 149 | Human | 19 | GUGAGAAGGAGCAAAGAUG | CAUCUUUGCUCCUUCUCAC | [272-290](19/19) | [272-290](19/19) | — | — |
| 150 | Human | 19 | GGAUAAGAAUGCGUCAACC | GGUUGACGCAUUCUUAUCC | [1014-1032](19/19) | [1125-1143](19/19) | — | — |
| 151 | Human | 19 | GGAUGGAUAAGAAUGCGUC | GACGCAUUCUUAUCCAUCC | [1010-1028](19/19) | [1121-1139](19/19) | — | — |
| 152 | Human | 19 | AGGAUCGGAUGGAUAAGAA | UUCUUAUCCAUCCGAUCCU | [1004-1022](19/19) | [1115-1133](19/19) | [1071-1089](18/19) | [830-848](18/19) |
| 153 | Human | 19 | UGAGUGAGAAGGAGCAAAG | CUUUGCUCCUUCUCACUCA | [269-287](19/19) | [269-287](19/19) | — | — |
| 154 | Human | 19 | AGAAGGAUCGGAUGGAUAA | UUAUCCAUCCGAUCCUUCU | [1001-1019](19/19) | [1112-1130](19/19) | [1068-1083](16/16) | [827-842](16/16) |
| 155 | Human | 19 | CAGAAGGAUCGGAUGGAUA | UAUCCAUCCGAUCCUUCUG | [1000-1018](19/19) | [1111-1129](19/19) | [1067-1083](17/17) | [826-842](17/17) |
| 156 | Human | 19 | CUACUCCAAAGGAUUCGGC | GCCGAAUCCUUUGGAGUAG | [966-984](19/19) | [1077-1095](19/19) | [1038-1051](14/14) | [797-810](14/14) |
| 157 | Human | 19 | GCAAGACUACUCCAAAGGA | UCCUUUGGAGUAGUCUUGC | [962-978(17/17) | [1071-1089](19/19) | [696-711](15/16) | [566-581](15/16) |
| 158 | Human | 19 | CCAGCAAGACUACUCCAAA | UUUGGAGUAGUCUUGCUGG | [962-975](14/14) | [1068-1086](19/19) | [1024-1035](12/12) | [450-466](16/17) |
| 159 | Human | 19 | CCCAGCAAGACUACUCCAA | UUGGAGUAGUCUUGCUGGG | [623-640](17/18) | [1067-1085](19/19) | [1023-1035](13/13) | [449-466](17/18) |
| 160 | Human | 19 | ACGAGUCCCAGCAAGACUA | UAGUCUUGCUGGGACUCGU | [839-857](18/19) | [1061-1079](19/19) | [1017-1035](18/19) | — |
| 161 | Human | 19 | ACAAGGAGAAGCUGGCCAA | UUGGCCAGCUUCUCCUUGU | — | [1040-1058](19/19) | [888-898](11/11) | [758-768](11/11) |
| 162 | Human | 19 | GGUUUGAUUACAAGGAGAA | UUCUCCUUGUAAUCAAACC | — | [1031-1049](19/19) | [987-1004](18/18) | — |
| 163 | Human | 19 | CUGUGGGGUUUGAUUACAA | UUGUAAUCAAACCCCACAG | — | [1025-1043](19/19) | [981-999](19/19) | [518-530](13/13) |
| 164 | Human | 19 | UGAAUGAUGUGAGUGAGAA | UUCUCACUCACAUCAUUCA | [260-278](19/19) | [260-278](19/19) | [216-231](16/16) | [86-101](15/16) |
| 165 | Human | 19 | CCACUUGCGUGAAAAAAAA | UUUUUUUUCACGCAAGUGG | [3127-3145](19/19) | — | — | — |
| 166 | Human | 19 | AAAGACUACUCCAAAGGAU | AUCCUUUGGAGUAGUCUUU | [961-979](19/19) | [1073-1090](18/18) | [695-711](16/17) | [565-581](16/17) |
| 167 | Human | 19 | ACCUCGCUAAGGAGAAAGA | UCUUUCUCCUUAGCGAGGU | [1130-1148](19/19) | [1241-1259](19/19) | — | — |
| 168 | Human | 19 | UUGAAAACCUCGCUAAGGA | UCCUUAGCGAGGUUUUCAA | [1124-1142](19/19) | [1235-1253](19/19) | — | — |
| 169 | Human | 19 | GAGCUAACUUUGAAAACCU | AGGUUUUCAAAGUUAGCUC | [1115-1133](19/19) | [1226-1244](19/19) | [1184-1200](17/17) | [943-955](13/13) |
| 170 | Human | 19 | CAUCAGAGCUAACUUUGAA | UUCAAAGUUAGCUCUGAUG | [1110-1128](19/19) | [1221-1239](19/19) | [1184-1195](12/12) | [943-954](12/12) |
| 171 | Human | 19 | CAAAACAAGUAACAUCAGA | UCUGAUGUUACUUGUUUUG | [1098-1116](19/19) | [1209-1227](19/19) | — | — |
| 172 | Human | 19 | AGCAAAACAAGUAACAUCA | UGAUGUUACUUGUUUUGCU | [1096-1114](19/19) | [1207-1225](19/19) | — | — |
| 173 | Human | 19 | ACCAGCAAAACAAGUAACA | UGUUACUUGUUUUGCUGGU | [1093-1111](19/19) | [1204-1222](19/19) | [1160-1176](16/17) | [919-935](16/17) |
| 174 | Human | 19 | UGACCAGCAAAACAAGUAA | UUACUUGUUUUGCUGGUCA | [1091-1109](19/19) | [1202-1220](19/19) | [1160-1176](16/17) | [917-935](18/19) |
| 175 | Human | 19 | GUGACCAGCAAAACAAGUA | UACUUGUUUUGCUGGUCAC | [1090-1108](19/19) | [1201-1219](19/19) | [1160-1175](15/16) | [916-934](18/19) |
| 176 | Human | 19 | CGAAGCUGUGACCAGCAAA | UUUGCUGGUCACAGCUUCG | [1083-1101](19/19) | [1194-1212](19/19) | — | [913-937](15/15) |
| 177 | Human | 19 | CAGUACCUGUCGAAGCUGU | ACAGCUUCGACAGGUACUG | [1073-1091](19/19) | [1184-1202](19/19) | — | — |
| 178 | Human | 19 | GACAGUACCUGUCGAAGCU | AGCUUCGACAGGUACUGUC | [1071-1089](19/19) | [1182-1200](19/19) | — | — |
| 179 | Human | 19 | CCAGAAGACAGUACCUGUC | GACAGGUACUGUCUUCUGG | [1065-1083](19/19) | [1176-1194](19/19) | — | — |
| FOR 21 | | | | | | | | |
| STRETCHES: | | | | | | | | |
| 1 | Cross Sp | 21 | GCUGCAUGAAUCCCAAAAAGA | UCUUUUUGGGAUUCAUGCAGC | [945-965](21/21) | [945-965](21/21) | [901-921](21/21) | [771-791](21/21) |
| 2 | Cross Sp | 21 | GUCUGCUGUAGGCUUUGAAUA | UAUUCAAAGCCUACAGCAGAC | [576-596](21/21) | [576-596](21/21) | [532-552](21/21) | [402-422](21/21) |
| 3 | Cross Sp | 21 | AAGUAUGGGGUGCAGAAGGAU | AUCCUUCUGCACCCCAUACUU | [988-1008](21/21) | [1099-1119](21/21) | [1055-1075](21/21) | [814-834](21/21) |
| 4 | Cross Sp | 21 | AGUCUGCUGUAGGCUUUGAAU | AUUCAAAGCCUACAGCAGACU | [575-595](21/21) | (575-595](21/21) | [531-551](21/21) | [401-421](21/21) |
| 5 | Cross Sp | 21 | CAGCUGCAUGAAUCCCAAAAA | UUUUUGGGAUUCAUGCAGCUG | [943-963](21/21) | [943-963](21/21) | [899-919](21/21) | [769-789](21/21) |
| 6 | Cross Sp | 21 | GCAGCUGCAUGAAUCCCAAAA | UUUUGGGAUUCAUGCAGCUGC | [942-962](21/21) | [942-962](21/21) | [898-918](21/21) | (768-788](21/21) |
| 7 | Cross Sp | 21 | GCUGUAGGCUUUGAAUACCAG | CUGGUAUUCAAAGCCUACAGC | [580-600](21/21) | (580-600](21/21) | [536-556](21/21) | [406-426](21/21) |
| 8 | Cross Sp | 21 | GAAGUAUGGGGUGCAGAAGGA | UCCUUCUGCACCCCAUACUUC | [987-1007](21/21) | [1098-1118](21/21) | [1054-1074](21/21) | [813-833](21/21) |
| 9 | Cross Sp | 21 | AAAGGAUUCGGCGGGAAGUAU | AUACUUCCCGCCGAAUCCUUU | [973-993](21/21) | [1084-1104](21/21) | [1040-1060](21/21) | [799-819](21/21) |
| 10 | Cross Sp | 21 | CAAAGGAUUCGGCGGGAAGUA | UACUUCCCGCCGAAUCCUUUG | [972-992](21/21) | [1083-1103](21/21) | [1039-1059](21/21) | [798-818](21/21) |
| 11 | Cross Sp | 21 | CAGUCUGCUGUAGGCUUUGAA | UUCAAAGCCUACAGCAGACUG | [574-594](21/21) | [574-594](21/21) | [530-550](21/21) | (400-420](21/21) |
| 12 | Cross Sp | 21 | UGCAGCUGCAUGAAUCCCAAA | UUUGGGAUUCAUGCAGCUGCA | [941-961](21/21) | [941-961](21/21) | [897-917](21/21) | [767-787](21/21) |
| 13 | Cross Sp | 21 | UUUGGUGUGCAGACAGACAGA | UCUGUCUGUCUGCACACCAAA | [880-900](21/21) | [880-900](21/21) | [836-856](21/21) | [706-726](21/21) |
| 14 | Cross Sp | 21 | GGCUUUGAAUACCAGGGGAAG | CUUCCCCUGGUAUUCAAAGCC | [586-606](21/21) | [586-606](21/21) | [542-562](21/21) | [412-432](21/21) |
| 15 | Cross Sp | 21 | GUAGGCUUUGAAUACCAGGGG | CCCCUGGUAUUCAAAGCCUAC | [583-603](21/21) | [583-603](21/21) | [539-559](21/21) | [409-429](21/21) |
| 16 | Cross Sp | 21 | CUGUAGGCUUUGAAUACCAGG | CCUGGUAUUCAAAGCCUACAG | [581-601](21/21) | [581-601](21/21) | [537-557](21/21) | [407-427](21/21) |
| 17 | Cross Sp | 21 | AUCAGUCUGCUGUAGGCUUUG | CAAAGCCUACAGCAGACUGAU | [572-592](21/21) | [572-592](21/21) | [528-548](21/21) | [398-418](21/21) |
| 18 | Cross Sp | 21 | UGCUGUAGGCUUUGAAUACCA | UGGUAUUCAAAGCCUACAGCA | [579-599](21/21) | [579-599](21/21) | [535-555](21/21) | [405-425](21/21) |
| 19 | Cross Sp | 21 | CUGCUGUAGGCUUUGAAUACC | GGUAUUCAAAGCCUACAGCAG | [578-598](21/21) | [578-598](21/21) | [534-554](21/21) | [404-424](21/21) |
| 20 | Cross Sp | 21 | UCUGCUGUAGGCUUUGAAUAC | GUAUUCAAAGCCUACAGCAGA | [577-597](21/21) | [577-597](21/21) | [533-553](21/21) | [403-423](21/21) |
| 21 | Cross Sp | 21 | AGCUGCAUGAAUCCCAAAAAG | CUUUUUGGGAUUCAUGCAGCU | [944-964](21/21) | [944-964](21/21) | [900-920](21/21) | [770-790](21/21) |
| 22 | Cross Sp | 21 | UGGUGUGCAGACAGACAGACA | UGUCUGUCUGUCUGCACACCA | [882-902](21/21) | [882-902](21/21) | [838-858](21/21) | [708-728](21/21) |
| 23 | Cross Sp | 21 | UCAGUCUGCUGUAGGCUUUGA | UCAAAGCCUACAGCAGACUGA | [573-593](21/21) | [573-593](21/21) | [529-549](21/21) | [399-419](21/21) |
| 24 | Cross Sp | 21 | GCUUUGAAUACCAGGGGAAGA | UCUUCCCCUGGUAUUCAAAGC | [587-607](21/21) | [587-607](21/21) | [543-563](21/21) | [413-433](21/21) |
| 25 | Cross Sp | 21 | AGGCUUUGAAUACCAGGGGAA | UUCCCCUGGUAUUCAAAGCCU | [585-605](21/21) | [585-605](21/21) | [541-561](21/21) | [411-431](21/21) |
| 26 | Cross Sp | 21 | UAGGCUUUGAAUACCAGGGGA | UCCCCUGGUAUUCAAAGCCUA | [584-604](21/21) | [584-604](21/21) | [540-560](21/21) | [410-430](21/21) |
| 27 | Cross Sp | 21 | GAUCAGUCUGCUGUAGGCUUU | AAAGCCUACAGCAGACUGAUC | [571-591](21/21) | [571-591](21/21) | [527-547](21/21) | [397-417](21/21) |
| 28 | Cross Sp | 21 | AGUAUGGGGUGCAGAAGGAUC | GAUCCUUCUGCACCCCAUACU | [989-1009](21/21) | [1100-1120](21/21) | [1056-1076](21/21) | [815-835](21/21) |
| 29 | Cross Sp | 21 | AAGGAUUCGGCGGGAAGUAUG | CAUACUUCCCGCCGAAUCCUU | [974-994](21/21) | [1085-1105](21/21) | [1041-1061](21/21) | [800-820](21/21) |
| 30 | Cross Sp | 21 | UUGGUGUGCAGACAGACAGAC | GUCUGUCUGUCUGCACACCAA | [881-901](21/21) | [881-901](21/21) | [837-857](21/21) | [707-727](21/21) |
| 31 | Cross Sp | 21 | CUUUGAAUACCAGGGGAAGAC | GUCUUCCCCUGGUAUUCAAAG | [588-608](21/21) | [588-608](21/21) | [544-564](21/21) | [414-434](21/21) |
| 32 | Cross Sp | 21 | UGUAGGCUUUGAAUACCAGGG | CCCUGGUAUUCAAAGCCUACA | [582-602](21/21) | [582-602](21/21) | [538-558](21/21) | [408-428](21/21) |
| 33 | Cross Sp | 21 | CCAAAGGAUUCGGCGGGAAGU | ACUUCCCGCCGAAUCCUUUGG | [971-991](21/21) | [1082-1102](21/21) | [1038-1058](21/21) | [797-817](21/21) |
| 34 | Cross Sp | 21 | CUACCAGGGCAAGACGGAGAA | UUCUCCGUCUUGCCCUGGUAG | [705-725](21/21) | [705-725](21/21) | [661-681](21/21) | [531-551](21/21) |
| 35 | Cross Sp | 21 | CAACUAUGUGGAGCUGCGGCA | UGCCGCAGCUCCACAUAGUUG | [1692-1712](21/21) | [1803-1823](21/21) | [1747-1767](21/21) | [1503-1523](21/21) |
| 36 | Cross Sp | 21 | GGAAGUAUGGGGUGCAGAAGG | CCUUCUGCACCCCAUACUUCC | [986-1006](21/21) | [1097-1117](21/21) | [1053-1073](21/21) | [812-832](21/21) |
| 37 | Cross Sp | 21 | GGGAAGUAUGGGGUGCAGAAG | CUUCUGCACCCCAUACUUCCC | [985-1005](21/21) | [1096-1116](21/21) | [1052-1072](21/21) | [811-831](21/21) |
| 38 | Cross Sp | 21 | UACCAGGGCAAGACGGAGAAG | CUUCUCCGUCUUGCCCUGGUA | [706-726](21/21) | [706-726](21/21) | [662-682](21/21) | [532-552](21/21) |
| 39 | Cross Sp | 21 | UAUGUGGAGCUGCGGCAGUAG | CUACUGCCGCAGCUCCACAUA | [1696-1716](21/21) | [1807-1827](21/21) | [1751-1771](21/21) | [1507-1527](21/21) |
| 40 | Cross Sp | 21 | CUAUGUGGAGCUGCGGCAGUA | UACUGCCGCAGCUCCACAUAG | [1695-1715](21/21) | [1806-1826](21/21) | [1750-1770](21/21) | [1506-1526](21/21) |
| 41 | Cross Sp | 21 | AACUAUGUGGAGCUGCGGCAG | CUGCCGCAGCUCCACAUAGUU | [1693-1713](21/21) | [1804-1824](21/21) | [1748-1768](21/21) | [1504-1524](21/21) |
| 42 | Cross Sp | 21 | GGUGCAGAAGGAUCGGAUGGA | UCCAUCCGAUCCUUCUGCACC | [996-1016](21/21) | [1107-1127](21/21) | [1063-1083](21/21) | [822-842](21/21) |
| 43 | Cross Sp | 21 | UAUGGGGUGCAGAAGGAUCGG | CCGAUCCUUCUGCACCCCAUA | [991-1011](21/21) | [1102-1122](21/21) | [1058-1078](21/21) | [817-837](21/21) |
| 44 | Cross Sp | 21 | CGGGAAGUAUGGGGUGCAGAA | UUCUGCACCCCAUACUUCCCG | [984-1004](21/21) | [1095-1115](21/21) | [1051-1071](21/21) | [810-830](21/21) |
| 45 | Cross Sp | 21 | AUUCGGCGGGAAGUAUGGGGU | ACCCCAUACUUCCCGCCGAAU | [978-998](21/21) | [1089-1109](21/21) | [1045-1065](21/21) | [804-824](21/21) |
| 46 | Cross Sp | 21 | AGGAUUCGGCGGGAAGUAUGG | CCAUACUUCCCGCCGAAUCCU | [975-995](21/21) | [1086-1106](21/21) | [1042-1062](21/21) | [801-821](21/21) |
| 47 | Cross Sp | 21 | ACUAUGUGGAGCUGCGGCAGU | ACUGCCGCAGCUCCACAUAGU | [1694-1714](21/21) | [1805-1825](21/21) | [1749-1769](21/21) | [1505-1525](21/21) |
| 48 | Cross Sp | 21 | UGGGGUGCAGAAGGAUCGGAU | AUCCGAUCCUUCUGCACCCCA | [993-1013](21/21) | [1104-1124](21/21) | [1060-1080](21/21) | [819-839](21/21) |
| 49 | Cross Sp | 21 | GUAUGGGGUGCAGAAGGAUCG | CGAUCCUUCUGCACCCCAUAC | [990-1010](21/21) | [1101-1121](21/21) | [1057-1077](21/21) | [816-836](21/21) |
| 50 | Cross Sp | 21 | AUGGGGUGCAGAAGGAUCGGA | UCCGAUCCUUCUGCACCCCAU | [992-1012](21/21) | [1103-1123](21/21) | [1059-1079](21/21) | [818-838](21/21) |
| 51 | Cross Sp | 21 | ACUACCAGGGCAAGACGGAGA | UCUCCGUCUUGCCCUGGUAGU | [704-724](21/21) | [704-724](21/21) | [660-680](21/21) | [530-550](21/21) |
| 52 | Human | 21 | GCAAGACUACUCCAAAGGAUU | AAUCCUUUGGAGUAGUCUUGC | [962-980](19/19) | [1071-1091](21/21) | [696-711](15/16) | [566-581](15/16) |
| 53 | Human | 21 | GCAGACAGACAGACAAGACAA | UUGUCUUGUCUGUCUGUCUGC | [888-908](21/21) | [888-908](21/21) | [844-864](20/21) | [714-734](20/21) |
| 54 | Human | 21 | GAAGCACGAGUCCCAGAAAGA | UCUUUCUGGGACUCGUGCUUC | [834-854](21/21) | [834-854](21/21) | [790-810](20/21) | [660-680](20/21) |
| 55 | Human | 21 | GAAGCAUGCCUCCCAGAAAGA | UCUUUCUGGGAGGCAUGCUUC | [612-632](21/21) | [612-632](21/21) | [568-588](21/21) | [438-458](20/21) |
| 56 | Human | 21 | GAAGGUCACGUGGAAAUGUCU | AGACAUUUCCACGUGACCUUC | [2914-2934](21/21) | [3024-3044](21/21) | — | — |
| 57 | Human | 21 | CCACUCACUUUGUAGGAAACU | AGUUUCCUACAAAGUGAGUGG | [2821-2841](21/21) | [2931-2951](21/21) | — | — |
| 58 | Human | 21 | CAAGACCGAAUGGAUAAGUCA | UGACUUAUCCAUUCGGUCUUG | [448-468](21/21) | [448-468](21/21) | — | — |
| 59 | Human | 21 | GGAUCCUCACACUACAGAUCA | UGAUCUGUAGUGUGAGGAUCC | [1748-1768](21/21) | [1859-1879](21/21) | [1809-1820](12/12) | — |
| 60 | Human | 21 | CCACAGAGGCUGUCUAUGAAA | UUUCAUAGACAGCCUCUGUGG | [1457-1477](21/21) | [1568-1588](21/21) | — | — |
| 61 | Human | 21 | UAAGAAUGCGUCAACCUUUGA | UCAAAGGUUGACGCAUUCUUA | [1017-1037](21/21) | [1128-1148](21/21) | — | — |
| 62 | Human | 21 | GGAUAAGAAUGCGUCAACCUU | AAGGUUGACGCAUUCUUAUCC | [1014-1034](21/21) | [1125-1145](21/21) | — | — |
| 63 | Human | 21 | GCAGAAGGAUCGGAUGGAUAA | UUAUCCAUCCGAUCCUUCUGC | [999-1019](21/21) | [1110-1130](21/21) | [1066-1083](18/18) | [825-842](18/18) |
| 64 | Human | 21 | GUGAGUGAGAAGGAGCAAAGA | UCUUUGCUCCUUCUCACUCAC | [268-288](21/21) | [268-288](21/21) | — | — |
| 65 | Human | 21 | CAGCAAGACUACUCCAAAGGA | UCCUUUGGAGUAGUCUUGCUG | [962-978](17/17) | [1069-1089](21/21) | [1025-1045](19/21) | [566-581](15/16) |
| 66 | Human | 21 | AGACUGGUUUUGGAGGCAAAU | AUUUGCCUCCAAAACCAGUCU | — | [971-991](21/21) | — | — |
| 67 | Human | 21 | GAGAAAUUGCAGCUGCAUGAA | UUCAUGCAGCUGCAAUUUCUC | [934-954](21/21) | [934-954](21/21) | [897-910](14/14) | [767-780](14/14) |
| 68 | Human | 21 | AGACAGACAGACAAGACAAAU | AUUUGUCUUGUCUGUCUGUCU | [890-910](21/21) | [890-910](21/21) | [846-864](18/19) | [716-734](18/19) |
| 69 | Human | 21 | UGAAAGGGUUUGGAGGAAAAU | AUUUUCCUCCAAACCCUUUCA | [860-880](21/21) | [860-880](21/21) | [818-834](16/17) | [688-704](16/17) |
| 70 | Human | 21 | AGACCGACCCUGAUUUUGUGA | UCACAAAAUCAGGGUCGGUCU | [242-262](21/21) | [242-262](21/21) | [206-218](13/13) | [68-88](20/21) |
| 71 | Human | 21 | CACGAGUCCCAGAAAGACUAU | AUAGUCUUUCUGGGACUCGUG | [838-858](21/21) | [838-858](21/21) | [794-814](20/21) | [667-684](18/18) |
| 72 | Human | 21 | GAGAAGCACGAGUCCCAGAAA | UUUCUGGGACUCGUGCUUCUC | [832-852](21/21) | [832-852](21/21) | [788-808](20/21) | [658-678](20/21) |
| 73 | Human | 21 | GCUUUGAGUAUCAAGGCAAAA | UUUUGCCUUGAUACUCAAAGC | [809-829](21/21) | [809-829](21/21) | [765-783](19/19) | [635-655](21/21) |
| 74 | Human | 21 | CCAAAGGUUUCGGCGGCAAAU | AUUUGCCGCCGAAACCUUUGG | [749-769](21/21) | [749-769](21/21) | — | — |
| 75 | Human | 21 | GAGAGAUUACUCCAAAGGUUU | AAACCUUUGGAGUAAUCUCUC | [738-758](21/21) | [738-758](21/21) | [697-714](18/18) | [567-584](18/18) |
| 76 | Human | 21 | CAGAGAGAUUACUCCAAAGGU | ACCUUUGGAGUAAUCUCUCUG | [736-756](21/21) | [736-756](21/21) | [692-712](20/21) | [562-582](20/21) |
| 77 | Human | 21 | GAAGCACGAGUCACAGAGAGA | UCUCUCUGUGACUCGUGCUUC | [723-743](21/21) | [723-743](21/21) | — | [549-565](17/17) |
| 78 | Human | 21 | GAAAGACUACUCCAGUGGUUU | AAACCACUGGAGUAGUCUUUC | [627-647](21/21) | [627-647](21/21) | [583-600](17/18) | [453-473](21/21) |
| 79 | Human | 21 | AGAUGGACAGAGUUGAUCAGU | ACUGAUCAACUCUGUCCAUCU | [557-577](21/21) | [557-577](21/21) | — | — |
| 80 | Human | 21 | CCAGAUGGACAGAGUUGAUCA | UGAUCAACUCUGUCCAUCUGG | [555-575](21/21) | [555-575](21/21) | [511-531](19/21) | [381-401](19/21) |
| 81 | Human | 21 | GGUGUCCAGAUGGACAGAGUU | AACUCUGUCCAUCUGGACACC | [550-570](21/21) | [550-570](21/21) | [506-522](17/17) | [376-392](17/17) |
| 82 | Human | 21 | GCAAGUUUGGUGUCCAGAUGG | CCAUCUGGACACCAAACUUGC | [542-562](21/21) | [542-562](21/21) | [498-518](20/21) | [368-388](20/21) |
| 83 | Human | 21 | CGAAUAUCAGUCGAAACUUUC | GAAAGUUUCGACUGAUAUUCG | [480-500](21/21) | [480-500](21/21) | — | — |
| 84 | Human | 21 | CACGAAUAUCAGUCGAAACUU | AAGUUUCGACUGAUAUUCGUG | [478-498](21/21) | [478-498](21/21) | — | — |
| 85 | Human | 21 | CACACCUCAUAGGUAUGAUUU | AAAUCAUACCUAUGAGGUGUG | [3069-3089](21/21) | [3179-3199](21/21) | — | — |
| 86 | Human | 21 | GUAGCACACCUCAUAGGUAUG | CAUACCUAUGAGGUGUGCUAC | [3065-3085](21/21) | [3175-3195](21/21) | — | — |
| 87 | Human | 21 | CCUGUAGCACACCUCAUAGGU | ACCUAUGAGGUGUGCUACAGG | [3062-3082](21/21) | [3172-3192](21/21) | — | — |
| 88 | Human | 21 | GUGCAUUUUCUCAUCAUCCUU | AAGGAUGAUGAGAAAAUGCAC | [2983-3003](21/21) | [3093-3113](21/21) | — | — |
| 89 | Human | 21 | CCAGAAGGUCACGUGGAAAUG | CAUUUCCACGUGACCUUCUGG | [2911-2931](21/21) | [3021-3041](21/21) | — | — |
| 90 | Human | 21 | CUCACUUUGUAGGAAACUCAU | AUGAGUUUCCUACAAAGUGAG | [2824-2844](21/21) | [2934-2954](21/21) | — | — |
| 91 | Human | 21 | GCAUUUGUGGCCACUCACUUU | AAAGUGAGUGGCCACAAAUGC | [2811-2831](21/21) | [2921-2941](21/21) | — | — |
| 92 | Human | 21 | GGAACAAGACCGAAUGGAUAA | UUAUCCAUUCGGUCUUGUUCC | [444-464](21/21) | [444-464](21/21) | — | — |
| 93 | Human | 21 | GUUGAAGGCUAGAAGUGUGAA | UUCACACUUCUAGCCUUCAAC | [2728-2748](21/21) | [2838-2858](21/21) | — | — |
| 94 | Human | 21 | GAUCAGUUGAAGGCUAGAAGU | ACUUCUAGCCUUCAACUGAUC | [2723-2743](21/21) | [2833-2853](21/21) | — | — |
| 95 | Human | 21 | UCAAUGGGGGUGUAGUAUUUU | AAAAUACUACACCCCCAUUGA | [2670-2690](21/21) | [2780-2800](21/21) | — | — |
| 96 | Human | 21 | UCACAAAGGCUGAUGUCUUAA | UUAAGACAUCAGCCUUUGUGA | [2516-2536](21/21) | [2626-2646](21/21) | — | — |
| 97 | Human | 21 | CCCAUGGCUAUGGAGGGAAAU | AUUUCCCUCCAUAGCCAUGGG | [416-436](21/21) | [416-436](21/21) | — | [242-260](18/19) |
| 98 | Human | 21 | GCAGAGACCCUGGUUUUUUUC | GAAAAAAACCAGGGUCUCUGC | [2309-2329](21/21) | [2419-2439](21/21) | — | — |
| 99 | Human | 21 | CUAGCAGAGACCCUGGUUUUU | AAAAACCAGGGUCUCUGCUAG | [2306-2326](21/21) | [2416-2436](21/21) | — | — |
| 100 | Human | 21 | GGAUCAGGUGACUUCUAGCAG | CUGCUAGAAGUCACCUGAUCC | [2292-2312](21/21) | [2402-2422](21/21) | — | — |
| 101 | Human | 21 | CUGCCAUGUCAGAUGGGAAAU | AUUUCCCAUCUGACAUGGCAG | [2242-2262](21/21) | [2352-2372](21/21) | — | — |
| 102 | Human | 21 | GGAACUUGAAACAGGACCAAA | UUUGGUCCUGUUUCAAGUUCC | [390-410](21/21) | [390-410](21/21) | — | — |
| 103 | Human | 21 | GAAGUUAACCCGGAGCUAAGU | ACUUAGCUCCGGGUUAACUUC | [2204-2224](21/21) | [2314-2334](21/21) | — | — |
| 104 | Human | 21 | CAGACCCUUAAGGAGAAGGAA | UUCCUUCUCCUUAAGGGUCUG | [373-393](21/21) | [373-393](21/21) | [329-348](18/20) | — |
| 105 | Human | 21 | GAGCUCGCAUUCUCUUGUGUU | AACACAAGAGAAUGCGAGCUC | [2027-2047](21/21) | [2137-2157](21/21) | — | — |
| 106 | Human | 21 | GGAGCUCGCAUUCUCUUGUGU | ACACAAGAGAAUGCGAGCUCC | [2026-2046](21/21) | [2136-2156](21/21) | — | — |
| 107 | Human | 21 | AGAGCAUCAGACCCUUAAGGA | UCCUUAAGGGUCUGAUGCUCU | [366-386](21/21) | [366-386](21/21) | — | — |
| 108 | Human | 21 | CAAGAGCAUCAGACCCUUAAG | CUUAAGGGUCUGAUGCUCUUG | [364-384](21/21) | [364-384](21/21) | — | — |
| 109 | Human | 21 | GGACUUUGGUAAUUGGUUUUA | UAAAACCAAUUACCAAAGUCC | [1947-1967](21/21) | [2057-2077](21/21) | — | — |
| 110 | Human | 21 | GGAGGACUUUGGUAAUUGGUU | AACCAAUUACCAAAGUCCUCC | [1944-1964](21/21) | [2054-2074](21/21) | — | — |
| 111 | Human | 21 | CCUUAGGAGGACUUUGGUAAU | AUUACCAAAGUCCUCCUAAGG | [1939-1959](21/21) | [2049-2069](21/21) | — | — |
| 112 | Human | 21 | CACAUUCCUUAGGAGGACUUU | AAAGUCCUCCUAAGGAAUGUG | [1933-1953](21/21) | [2043-2063](21/21) | — | — |
| 113 | Human | 21 | GGGAAUAUACACAUUGCUUUU | AAAAGCAAUGUGUAUAUUCCC | [1845-1865](21/21) | [1955-1975](21/21) | — | — |
| 114 | Human | 21 | GAAGGUGGGGAGGGGAAUAUA | UAUAUUCCCCUCCCCACCUUC | [1833-1853](21/21) | [1943-1963](21/21) | — | — |
| 115 | Human | 21 | CUGGAUCCUCACACUACAGAU | AUCUGUAGUGUGAGGAUCCAG | [1746-1766](21/21) | [1857-1877](21/21) | [1809-1820](12/12) | — |
| 116 | Human | 21 | CGAUGAUGAGAUCUCAUUUGA | UCAAAUGAGAUCUCAUCAUCG | [1584-1604](21/21) | [1695-1715](21/21) | [1639-1659](20/21) | — |
| 117 | Human | 21 | GGCGAUGAUGAGAUCUCAUUU | AAAUGAGAUCUCAUCAUCGCC | [1582-1602](21/21) | [1693-1713](21/21) | [1637-1653](17/17) | [1393-1409](16/17) |
| 118 | Human | 21 | GGAGCAUAUCAACAUACACAA | UUGUGUAUGUUGAUAUGCUCC | [324-344](21/21) | [324-344](21/21) | — | [157-170](14/14) |
| 119 | Human | 21 | CCAGGAGCAUAUCAACAUACA | UGUAUGUUGAUAUGCUCCUGG | [321-341](21/21) | [321-341](21/21) | — | [147-167](19/21) |
| 120 | Human | 21 | CCAGCAAAACAAGUAACAUCA | UGAUGUUACUUGUUUUGCUGG | [1094-1114](21/21) | [1205-1225](21/21) | — | — |
| 121 | Human | 21 | GACCAGCAAAACAAGUAACAU | AUGUUACUUGUUUUGCUGGUC | [1092-1112](21/21) | [1203-1223](21/21) | [1160-1176](16/17) | [918-935](17/18) |
| 122 | Human | 21 | CGAAGCUGUGACCAGCAAAAC | GUUUUGCUGGUCACAGCUUCG | [1083-1103](21/21) | [1194-1214](21/21) | [1160-1170](11/11) | [913-929](17/17) |
| 123 | Human | 21 | GUCGAAGCUGUGACCAGCAAA | UUUGCUGGUCACAGCUUCGAC | [1081-1101](21/21) | [1192-1212](21/21) | — | [913-927](15/15) |
| 124 | Human | 21 | CUACCAGAAGACAGUACCUGU | ACAGGUACUGUCUUCUGGUAG | [1062-1082](21/21) | [1173-1193](21/21) | — | — |
| 125 | Human | 21 | GCCUACCAGAAGACAGUACCU | AGGUACUGUCUUCUGGUAGGC | [1060-1080](21/21) | [1171-1191](21/21) | — | — |
| 126 | Human | 21 | UCUGCCUACCAGAAGACAGUA | UACUGUCUUCUGGUAGGCAGA | [1057-1077](21/21) | [1168-1188](21/21) | — | — |
| 127 | Human | 21 | UCCUCUGCCUACCAGAAGACA | UGUCUUGUGGUAGGCAGAGGA | [1054-1074](21/21) | [1165-1185](21/21) | — | — |
| 128 | Human | 21 | CGUCAACCUUUGAGGAUGUCA | UGACAUCCUCAAAGGUUGACG | [1025-1045](21/21) | [1136-1156](21/21) | — | — |
| 129 | Human | 21 | GAAUGCGUCAACCUUUGAGGA | UCCUCAAAGGUUGACGCAUUC | [1020-1040](21/21) | [1131-1151](21/21) | — | — |
| 130 | Human | 21 | CGGAUGGAUAAGAAUGCGUCA | UGACGCAUUCUUAUCCAUCCG | [1009-1029](21/21) | [1120-1140](21/21) | — | — |
| 131 | Human | 21 | AAGGAUCGGAUGGAUAAGAAU | AUUCUUAUCCAUCCGAUCCUU | [1003-1023](21/21) | [1114-1134](21/21) | [1070-1090](20/21) | [829-848](19/20) |
| 132 | Human | 21 | GAAGGAUCGGAUGGAUAAGAA | UUCUUAUCCAUCCGAUCCUUC | [1002-1022](21/21) | [1113-1133](21/21) | [1069-1089](20/21) | [828-848](20/21) |
| 133 | Human | 21 | GAUGUGAGUGAGAAGGAGCAA | UUGCUCCUUCUCACUCACAUC | [265-285](21/21) | [265-285](21/21) | [221-240](19/20) | [91-107](16/17) |
| 134 | Human | 21 | CAAGACUACUCCAAAGGAUUC | GAAUCCUUUGGAGUAGUCUUG | [962-981](20/20) | [1072-1092](21/21) | [1028-1048](19/21) | [788-807](18/20) |
| 135 | Human | 21 | CCAGCAAGACUACUCCAAAGG | CCUUUGGAGUAGUCUUGCUGG | [962-977](16/16) | [1068-1088](21/21) | [1024-1044](19/21) | [450-466](16/17) |
| 136 | Human | 21 | UCCCAGCAAGACUACUCCAAA | UUUGGAGUAGUCUUGCUGGGA | [622-640](18/19) | [1066-1086](21/21) | [1023-1035](13/13) | [448-466](18/19) |
| 137 | Human | 21 | GGGUUUGAUUACAAGGAGAAG | CUUCUCCUUGUAAUCAAACCC | — | [1030-1050](21/21) | [986-1004](19/19) | — |
| 138 | Human | 21 | CUGUGGGGUUUGAUUACAAGG | CCUUGUAAUCAAACCCCACAG | — | [1025-1045](21/21) | [981-1001](21/21) | [518-530](13/13) |
| 139 | Human | 21 | CGCUGCUGUGGGGUUUGAUUA | UAAUCAAACCCCACAGCAGCG | — | [1020-1040](21/21) | [980-996](17/17) | [516-530](15/15) |
| 140 | Human | 21 | GCAAAUUCGGUGUUCAGUCGG | CCGACUGAACACCGAAUUUGC | — | [986-1006](21/21) | [942-960](18/19) | — |
| 141 | Human | 21 | GAGGCAAAUUCGGUGUUCAGU | ACUGAACACCGAAUUUGCCUC | — | [983-1003](21/21) | — | — |
| 142 | Human | $$ | GGAGGCAAAUUCGGUGUUCAG | CUGAACACCGAAUUUGCCUCC | — | [982-1002](21/21) | [938-958](20/21) | — |
| 143 | Human | 21 | CUGGUUUUGGAGGCAAAUUCG | CGAAUUUGCCUCCAAAACCAG | — | [974-994](21/21) | [930-948](18/19) | — |
| 144 | Human | 21 | GCAUGAAUCCCAAAAAGAUUA | UAAUCUUUUUGGGAUUCAUGC | [948-965](18/18) | [948-968](21/21) | [904-921](18/18) | [774-791](18/18) |
| 145 | Human | 21 | CUGCAUGAAUCCCAAAAAGAU | AUCUUUUUGGGAUUCAUGCAG | [946-965](20/20) | [946-966](21/21) | [902-921](20/20) | [772-791](20/20) |
| 146 | Human | 21 | CAGGAGAAAUUGCAGCUGCAU | AUGCAGCUGCAAUUUCUCCUG | [931-951](21/21) | [931-951](21/21) | [887-907](19/21) | [757-777](19/21) |
| 147 | Human | 21 | CCAGGAGAAAUUGCAGCUGCA | UGCAGCUGCAAUUUCUCCUGG | [930-950](21/21) | [930-950](21/21) | — | — |
| 148 | Human | 21 | CAAAAAGACUACUCCAAAGGA | UCCUUUGGAGUAGUCUUUUUG | [958-978](21/21) | [1073-1089](17/17) | [695-711](16/17) | [784-804](19/21) |
| 149 | Human | 21 | CAUCAGAGCUAACUUUGAAAA | UUUUCAAAGUUAGCUCUGAUG | [1110-1130](21/21) | [1221-1241](21/21) | [1184-1197](14/14) | [943-955](13/13) |
| 150 | Human | 21 | ACAUCAGAGCUAACUUUGAAA | UUUCAAAGUUAGCUCUGAUGU | [1109-1129](21/21) | [1220-1240](21/21) | [1184-1196](13/13) | [943-955](13/13) |
| 151 | Human | 21 | UAACAUCAGAGCUAACUUUGA | UCAAAGUUAGCUCUGAUGUUA | [1107-1127](21/21) | [1218-1238](21/21) | [1184-1194](11/11) | [943-953](11/11) |
| 152 | Human | 21 | GUAACAUCAGAGCUAACUUUG | CAAAGUUAGCUCUGAUGUUAC | [1106-1126](21/21) | [1217-1237](21/21) | — | — |
| 153 | Human | 21 | CAAGUAACAUCAGAGCUAACU | AGUUAGCUCUGAUGUUACUUG | [1103-1123](21/21) | [1214-1234](21/21) | — | — |
| 154 | Human | 21 | GCAAAACAAGUAACAUCAGAG | CUCUGAUGUUACUUGUUUUGC | [1097-1117](21/21) | [1208-1228](21/21) | — | — |
| 155 | Human | 21 | CAGCAAAACAAGUAACAUCAG | CUGAUGUUACUUGUUUUGCUG | [1095-1115](21/21) | [1206-1226](21/21) | — | — |
| 156 | Human | 21 | UGACCAGCAAAACAAGUAACA | UGUUACUUGUUUUGCUGGUCA | [1091-1111](21/21) | [1202-1222](21/21) | [1160-1176](16/17) | [917-935](18/19) |
| 157 | Human | 21 | GCUGUGACCAGCAAAACAAGU | ACUUGUUUUGCUGGUCACAGC | [1087-1107](21/21) | [1198-1218](21/21) | [1160-1170](11/11) | [913-929](17/17) |
| 158 | Human | 21 | GAAGCUGUGACCAGCAAAACA | UGUUUUGCUGGUCACAGCUUC | [1084-1104](21/21) | [1195-1215](21/21) | [1160-1170](11/11) | [913-929](17/17) |
| 159 | Human | 21 | AGACAGUACCUGUCGAAGCUG | CAGCUUCGACAGGUACUGUCU | [1070-1090](21/21) | [1181-1201](21/21) | — | — |
| 160 | Human | 21 | AGAAGACAGUACCUGUCGAAG | CUUCGACAGGUACUGUCUUCU | [1067-1087](21/21) | [1178-1198](21/21) | — | — |
| 161 | Human | 21 | AAAAGACUACUCCAAAGGAUU | AAUCCUUUGGAGUAGUCUUUU | [960-980](21/21) | [1073-1091](19/19) | [695-711](16/17) | [565-581](16/17) |
| 162 | Human | 21 | CCAAAAAGACUACUCCAAAGG | CCUUUGGAGUAGUCUUUUUGG | [957-977](21/21) | [1073-1088](16/16) | [695-711](16/17) | [783-803](19/21) |
| 163 | Human | 21 | UCCCAAAAAGACUACUCCAAA | UUUGGAGUAUUCUUUUUGGGA | [955-975](21/21) | [622-640](18/19) | [911-924](14/14) | [448-466](18/19) |
| 164 | Human | 21 | GCAUGAAUCCCAAAAAGACUA | UAGUCUUUUUGGGAUUCAUGC | [948-968](21/21) | [948-965](18/18) | [904-924](21/21) | [774-794](21/21) |

Table D below shows further siRNA compounds specific for the FXYD5 gene that have been selected according to the present invention

TABLE D

| | | | | | |
|---|---|---|---|---|---|
| | | Oligo | | | |
| No | Source | Length | Sense siRNA | AntiSense siRNA | human 47778937 |

FOR 19
STRETCHES:

| | | | | | |
|---|---|---|---|---|---|
| 1 | Human | 19 | GGAAGACACAGAUGAUGAA | UUCAUCAUCUGUGUCUUCC | [1474-1492](19/19) |
| 2 | Human | 19 | CCAGAGGACAGACGUUGAA | UUCAACGUCUGUCCUCUGG | [883-901](19/19) |
| 3 | Human | 19 | CAAGCCAUCUGGUUUUCAU | AUGAAAACCAGAUGGCUUG | [1199-1217](19/19) |
| 4 | Human | 19 | CAACUAUCAUGGACAUUCA | UGAAUGUCCAUGAUAGUUG | [931-949](19/19) |
| 5 | Human | 19 | GCAGACUCAACUAUCAUGG | CCAUGAUAGUUGAGUCUGC | [924-942](19/19) |
| 6 | Human | 19 | UCAGCAGACUCAACUAUCA | UGAUGAUUGAGUCUGCUGA | [921-939](19/19) |
| 7 | Human | 19 | CUUCAGCAGACUCAACUAU | AUAGUUGAGUCUGCUGAAG | [919-937](19/19) |
| 8 | Human | 19 | CAAGUUAUCUCCUCUGCUA | UAGCAGAGGAGAUAACUUG | [1582-1600](19/19) |
| 9 | Human | 19 | AGAGGGAAGACACAGAUGA | UCAUCUGUGUCUUCCCUCU | [1470-1488](19/19) |
| 10 | Human | 19 | AGAGAGGGAAGACACAGAU | AUCUGUGUCUUCCCUCUCU | [1468-1486](19/19) |
| 11 | Human | 19 | GGACAGACGUUGAAAGAUA | UAUCUUUCAACGUCUGUCC | [888-906](19/19) |
| 12 | Human | 19 | CCAUCGUUGGCCUGAUUCU | AGAAUCAGGCCAACGAUGG | [859-877](19/19) |
| 13 | Human | 19 | CACCAUCGUUGGCCUGAUU | AAUCAGGCCAACGAUGGUG | [857-875](19/19) |
| 14 | Human | 19 | GAUCCAGAGACACACAAGA | UCUUGUGUGUCUCUGGAUC | [1089-1107](19/19) |
| 15 | Human | 19 | CAGAUCCAGAGACACACAA | UUGUGUGUCUCUGGAUCUG | [1087-1105](19/19) |
| 16 | Human | 19 | GCAACUGGAAGGAACGGAU | AUCCGUUCCUUCCAGUUGC | [1055-1073](19/19) |
| 17 | Human | 19 | CUGGCCUGCUGAUGAAACA | UGUUUCAUCAGCAGGCCAG | [1013-1031](19/19) |
| 18 | Human | 19 | CCCAGAUGCAGUCUACACA | UGUGUAGACUGCAUCUGGG | [965-983](19/19) |
| 19 | Human | 19 | UUCUUCAGCAGACUCAACU | AGUUGAGUCUGCUGAAGAA | [917-935](19/19) |
| 20 | Human | 19 | CAGUUCUUCAGCAGACUCA | UGAGUCUGCUGAAGAACUG | [914-932](19/19) |
| 21 | Human | 19 | AAAGCACUGUGGUCUUUGC | GCAAAGACCACAGUGCUUU | [1612-1630](19/19) |
| 22 | Human | 19 | GUAAAGCACUGUGGUCUUU | AAAGACCACAGUGCUUUAC | [1610-1628](19/19) |
| 23 | Human | 19 | GAUACCACGUCCAGUUCUU | AAGAACUGGACGUGGUAUC | [903-921](19/19) |
| 24 | Human | 19 | UCUGCUAAGACAAAAAGUA | UACUUUUUGUCUUAGCAGA | [1594-1612](19/19) |
| 25 | Human | 19 | CCUCUGCUAAGACAAAAAG | CUUUUUGUCUUAGCAGAGG | [1592-1610](19/19) |
| 26 | Human | 19 | UAUCUCCUCUGCUAAGACA | UGUCUUAGCAGAGGAGAUA | [1587-1605](19/19) |
| 27 | Human | 19 | GGCUGUCCCUCAAGUUAUC | GAUAACUUGAGGGACAGCC | [1572-1590](19/19) |
| 28 | Human | 19 | AAAGAUACCACGUCCAGUU | AACUGGACGUGGUAUCUUU | [900-918](19/19) |
| 29 | Human | 19 | CACAGAUGAUGAAGCUGGA | UCCAGCUUCAUCAUCUGUG | [1480-1498](19/19) |
| 30 | Human | 19 | AGACACAGAUGAUGAAGCU | AGCUUCAUCAUCUGUGUCU | [1477-1495](19/19) |
| 31 | Human | 19 | GACAGACGUUGAAAGAUAC | GUAUCUUUCAACGUCUGUC | [889-907](19/19) |
| 32 | Human | 19 | GAGGACAGACGUUGAAAGA | UCUUUCAACGUCUGUCCUC | [886-904](19/19) |
| 33 | Human | 19 | AGAGGACAGACGUUGAAAG | CUUUCAACGUCUGUCCUCU | [885-903](19/19) |
| 34 | Human | 19 | AGAAACAGGAGCUGACAAC | GUUGUCAGCUCCUGUUUCU | [1372-1390](19/19) |
| 35 | Human | 19 | CGGUUAUGCCGGAAUCGUU | AACGAUUCCGGCAUAACCG | [1338-1356](19/19) |
| 36 | Human | 19 | GCAUCAUCAUCCUCACCAG | CUGGUGAGGAUGAUGAUGC | [1297-1315](19/19) |
| 37 | Human | 19 | UCACAGGCAUCAUCAUCCU | AGGAUGAUGAUGCCUGUGA | [1291-1309](19/19) |
| 38 | Human | 19 | UCAUCACAGGCAUCAUCAU | AUGAUGAUGCCUGUGAUGA | [1288-1306](19/19) |
| 39 | Human | 19 | GUUCAUCACAGGCAUCAUC | GAUGAUGCCUGUGAUGAAC | [1286-1304](19/19) |
| 40 | Human | 19 | CAGCUGUGCUGUUCAUCAC | GUGAUGAACAGCACAGCUG | [1276-1294](19/19) |
| 41 | Human | 19 | GCAGCUGUGCUGUUCAUCA | UGAUGAACAGCACAGCUGC | [1275-1293](19/19) |
| 42 | Human | 19 | UCGCAGCUGUGCUGUUCAU | AUGAACAGCACAGCUGCGA | [1273-1291](19/19) |
| 43 | Human | 19 | CUUCUUCUAUGAUGAACAC | GUGUUCAUCAUAGAAGAAG | [1229-1247](19/19) |
| 44 | Human | 19 | CCUUCUUCUAUGAUGAACA | UGUUCAUCAUAGAAGAAGG | [1228-1246](19/19) |
| 45 | Human | 19 | ACCCCUUCUUCUAUGAUGA | UCAUCAUAGAAGAAGGGGU | [1225-1243](19/19) |
| 46 | Human | 19 | AUGACCCCUUCUUCUAUGA | UCAUAGAAGAAGGGGUCAU | [1222-1240](19/19) |
| 47 | Human | 19 | GGAUGACCCCUUCUUCUAU | AUAGAAGAAGGGGUCAUCC | [1220-1238](19/19) |
| 48 | Human | 19 | GAGGAUGACCCCUUCUUCU | AGAAGAAGGGGUCAUCCUC | [1218-1236](19/19) |
| 49 | Human | 19 | AUGAGGAUGACCCCUUCUU | AAGAAGGGGUCAUCCUCAU | [1216-1234](19/19) |
| 50 | Human | 19 | CCAUCUGGUUUUCAUGAGG | CCUCAUGAAAACCAGAUGG | [1203-1221](19/19) |
| 51 | Human | 19 | CCUCAAGCCAUCUGGUUUU | AAAACCAGAUGGCUUGAGG | [1196-1214](19/19) |
| 52 | Human | 19 | GCUCAUCCCACUGAUGACA | UGUCAUCAGUGGGAUGAGC | [1119-1137](19/19) |
| 53 | Human | 19 | CAGCUCAUCCCACUGAUGA | UCAUCAGUGGGAUGAGCUG | [1117-1135](19/19) |
| 54 | Human | 19 | AGCAGCUCAUCCCACUGAU | AUCAGUGGGAUGAGCUGCU | [1115-1133](19/19) |
| 55 | Human | 19 | CAAAGCAGCUCAUCCCACU | AGUGGGAUGAGCUGCUUUG | [1112-1130](19/19) |
| 56 | Human | 19 | GAGCACCAAAGCAGCUCAU | AUGAGCUGCUUUGGUGCUC | [1106-1124](19/19) |
| 57 | Human | 19 | CACAAGAGCACCAAAGCAG | CUGCUUUGGUGCUCUUGUG | [1101-1119](19/19) |
| 58 | Human | 19 | ACACACAAGAGCACCAAAG | CUUUGGUGCUCUUGUGUGU | [1098-1116](19/19) |
| 59 | Human | 19 | GCUGAUGAAACACCACAAC | GUUGUGGUGUUUCAUCAGC | [1020-1038](19/19) |
| 60 | Human | 19 | GCCUGCUGAUGAAACACCA | UGGUGUUUCAUCAGCAGGC | [1016-1034](19/19) |
| 61 | Human | 19 | AACCUGGCCUGCUGAUGAA | UUCAUCAGCAGGCCAGGUU | [1010-1028](19/19) |
| 62 | Human | 19 | CCUGUGUCUUCUCACCAUC | GAUGGUGAGAAGACACAGG | [845-863](19/19) |
| 63 | Human | 19 | GCAGUCUACACAGAACUCC | GGAGUUCUGUGUAGACUGC | [972-990](19/19) |
| 64 | Human | 19 | AUGCAGUCUACACAGAACU | AGUUCUGUGUAGACUGCAU | [970-988](19/19) |
| 65 | Human | 19 | CAGAUGCAGUCUACACAGA | UCUGUGUAGACUGCAUCUG | [967-985](19/19) |
| 66 | Human | 19 | CUAUCAUGGACAUUCAGGU | ACCUGAAUGUCCAUGAUAG | [934-952](19/19) |
| 67 | Human | 19 | GACUCAACUAUCAUGGACA | UGUCCAUGAUAGUUGAGUC | [927-945](19/19) |
| 68 | Human | 19 | AGACUCAACUAUCAUGGAC | GUCCAUGAUAGUUGAGUCU | [926-944](19/19) |
| 69 | Human | 19 | CAGCAGACUCAACUAUCAU | AUGAUAGUUGAGUCUGCUG | [922-940](19/19) |
| 70 | Human | 19 | CCAGUUCUUCAGCAGACUC | GAGUCUGCUGAAGAACUGG | [913-931](19/19) |
| 71 | Human | 19 | CACGUCCAGUUCUUCAGCA | UGCUGAAGAACUGGACGUG | [908-926](19/19) |
| 72 | Human | 19 | ACCACGUCCAGUUCUUCAG | CUGAAGAACUGGACGUGGU | [906-924](19/19) |
| 73 | Human | 19 | CAAAAAGUAAAGCACUGUG | CACAGUGCUUUACUUUUUG | [1604-1622](19/19) |
| 74 | Human | 19 | AAGACAAAAAGUAAAGCAC | GUGCUUUACUUUUUGUCUU | [1600-1618](19/19) |
| 75 | Human | 19 | GCUAAGACAAAAAGUAAAG | CUUUACUUUUUGUCUUAGC | [1597-1615](19/19) |
| 76 | Human | 19 | CUGCUAAGACAAAAAGUAA | UUACUUUUUGUCUUAGCAG | [1595-1613](19/19) |
| 77 | Human | 19 | CUCUGCUAAGACAAAAAGU | ACUUUUUGUCUUAGCAGAG | [1593-1611](19/19) |
| 78 | Human | 19 | UCCUCUGCUAAGACAAAAA | UUUUUGUCUUAGCAGAGGA | [1591-1609](19/19) |
| 79 | Human | 19 | CUCCUCUGCUAAGACAAAA | UUUUGUCUUAGCAGAGGAG | [1590-1608](19/19) |
| 80 | Human | 19 | AUCUCCUCUGCUAAGACAA | UUGUCUUAGCAGAGGAGAU | [1588-1606](19/19) |
| 81 | Human | 19 | GUUAUCUCCUCUGCUAAGA | UCUUAGCAGAGGAGAUAAC | [1585-1603](19/19) |
| 82 | Human | 19 | AGAUACCACGUCCAGUUCU | AGAACUGGACGUGGUAUCU | [902-920](19/19) |
| 83 | Human | 19 | AAGUUAUCUCCUCUGCUAA | UUAGCAGAGGAGAUAACUU | [1583-1601](19/19) |
| 84 | Human | 19 | CUCAAGUUAUCUCCUCUGC | GCAGAGGAGAUAACUUGAG | [1580-1598](19/19) |
| 85 | Human | 19 | CCCUCAAGUUAUCUCCUCU | AGAGGAGAUAACUUGAGGG | [1578-1596](19/19) |
| 86 | Human | 19 | UGUCCCUCAAGUUAUCUCC | GGAGAUAACUUGAGGGACA | [1575-1593](19/19) |
| 87 | Human | 19 | CUGUCCCUCAAGUUAUCUC | GAGAUAACUUGAGGGACAG | [1574-1592](19/19) |
| 88 | Human | 19 | GGGCUGUCCCUCAAGUUAU | AUAACUUGAGGGACAGCCC | [1571-1589](19/19) |
| 89 | Human | 19 | UUGAAAGAUACCACGUCCA | UGGACGUGGUAUCUUUCAA | [897-915](19/19) |
| 90 | Human | 19 | GACGUUGAAAGAUACCACG | CGUGGUAUCUUUCAACGUC | [893-911](19/19) |
| 91 | Human | 19 | AGACGUUGAAAGAUACCAC | GUGGUAUCUUUCAACGUCU | [892-910](19/19) |
| 92 | Human | 19 | AGAUGAUGAAGCUGGAGCC | GGCUCCAGCUUCAUCAUCU | [1483-1501](19/19) |
| 93 | Human | 19 | CAGACGUUGAAAGAUACCA | UGGUAUCUUUCAACGUCUG | [891-909](19/19) |
| 94 | Human | 19 | GGGAAGACACAGAUGAUGA | UCAUCAUCUGUGUCUUCCC | [1473-1491](19/19) |
| 95 | Human | 19 | GAGGGAAGACACAGAUGAU | AUCAUCUGUGUCUUCCCUC | [1471-1489](19/19) |
| 96 | Human | 19 | CAGAGAGGGAAGACACAGA | UCUGUGUCUUCCCUCUCUG | [1467-1485](19/19) |
| 97 | Human | 19 | AACAGGAGCUGACAACCUG | CAGGUUGUCAGCUCCUGUU | [1375-1393](19/19) |
| 98 | Human | 19 | CAGAAACAGGAGCUGACAA | UUGUCAGCUCCUGUUUCUG | [1371-1389](19/19) |
| 99 | Human | 19 | CCAUCAGAAACAGGAGCUG | CAGCUCCUGUUUCUGAUGG | [1367-1385](19/19) |
| 100 | Human | 19 | GGUUAUGCCGGAAUCGUUG | CAACGAUUCCGGCAUAACC | [1339-1357](19/19) |

FOR 21
STRETCHES:

| | | | | | |
|---|---|---|---|---|---|
| 1 | Human | 21 | CCAGUUCUUCAGCAGACUCAA | UUGAGUCUGCUGAAGAACUGG | [913-933](21/21) |
| 2 | Human | 21 | GCAGCUGUGCUGUUCAUCACA | UGUGAUGAACAGCACAGCUGC | [1275-1295](21/21) |
| 3 | Human | 21 | CAACCUGGCCUGCUGAUGAAA | UUUCAUCAGCAGGCCAGGUUG | [1009-1029](21/21) |
| 4 | Human | 21 | CCAGAUGCAGUCUACACAGAA | UUCUGUGUAGACUGCAUCUGG | [966-986](21/21) |
| 5 | Human | 21 | GACUCAACUAUCAUGGACAUU | AAUGUCCAUGAUAGUUGAGUC | [927-947](21/21) |
| 6 | Human | 21 | GUUCUUCAGCAGACUCAACUA | UAGUUGAGUCUGCUGAAGAAC | [916-936](21/21) |
| 7 | Human | 21 | CUCUGCUAAGACAAAAAGUAA | UUACUUUUUGUCUUAGCAGAG | [1593-1613](21/21) |
| 8 | Human | 21 | UCAAGUUAUCUCCUCUGCUAA | UUAGCAGAGGAGAUAACUUGA | [1581-1601](21/21) |
| 9 | Human | 21 | GGGCUGUCCCUCAAGUUAUCU | AGAUAACUUGAGGGACAGCCC | [1571-1591](21/21) |
| 10 | Human | 21 | GAGAGGGAAGACACAGAUGAU | AUCAUCUGUGUCUUCCCUCUC | [1469-1489](21/21) |
| 11 | Human | 21 | CCAGAGAGGGAAGACACAGAU | AUCUGUGUCUUCCCUCUCUGG | [1466-1486](21/21) |
| 12 | Human | 21 | GGACAGACGUUGAAAGAUACC | GGUAUCUUUCAACGUCUGUCC | [888-908](21/21) |
| 13 | Human | 21 | CAGAGGACAGACGUUGAAAGA | UCUUUCAACGUCUGUCCUCUG | [881-904](21/21) |
| 14 | Human | 21 | CCUUCUUCUAUGAUGAACACA | UGUGUUCAUCAUAGAAGAAGG | [1228-1248](21/21) |
| 15 | Human | 21 | GACCCCUUCUUCUAUGAUGAA | UUCAUCAUAGAAGAAGGGGUC | [1224-1244](21/21) |
| 16 | Human | 21 | GAUGACCCCUUCUUCUAUGAU | AUCAUAGAAGAAGGGGUCAUC | [1221-1241](21/21) |
| 17 | Human | 21 | GGAUGACCCCUUCUUCUAUGA | UCAUAGAAGAAGGGGUCAUCC | [1220-1240](21/21) |
| 18 | Human | 21 | CAAGCCAUCUGGUUUUCAUGA | UCAUGAAAACCAGAUGGCUUG | [1199-1219](21/21) |
| 19 | Human | 21 | CUGUGUCUUCUCACCAUCGUU | AACGAUGGUGAGAAGACACAG | [846-866](21/21) |
| 20 | Human | 21 | UUCAGCAGACUCAACUAUCAU | AUGAUAGUUGAGUCUGCUGAA | [920-940](21/21) |
| 21 | Human | 21 | CACGUCCAGUUCUUCAGCAGA | UCUGCUGAAGAACUGGACGUG | [908-928](21/21) |
| 22 | Human | 21 | CAAAAAGUAAAGCACUGUGGU | ACCACAGUGCUUUACUUUUUG | [1604-1624](21/21) |
| 23 | Human | 21 | UCCUCUGCUAAGACAAAAAGU | ACUUUUUGUCUUAGCAGAGGA | [1591-1611](21/21) |
| 24 | Human | 21 | AGAUACCACGUCCAGUUCUUC | GAAGAACUGGACGUGGUAUCU | [902-922](21/21) |
| 25 | Human | 21 | UUAUCUCCUCUGCUAAGACAA | UUGUCUUAGCAGAGGAGAUAA | [1586-1606](21/21) |
| 26 | Human | 21 | CAAGUUAUCUCCUCUGCUAAG | CUUAGCAGAGGAGAUAACUUG | [1582-1602](21/21) |
| 27 | Human | 21 | CCUCAAGUUAUCUCCUCUGCU | AGCAGAGGAGAUAACUUGAGG | [1579-1599](21/21) |
| 28 | Human | 21 | CUGUCCCUCAAGUUAUCUCCU | AGGAGAUAACUUGAGGGACAG | [1574-1594](21/21) |
| 29 | Human | 21 | AAAGAUACCACGUCCAGUUCU | AGAACUGGACGUGGUAUCUUU | [900-920](21/21) |
| 30 | Human | 21 | GAAAGAUACCACGUCCAGUUC | GAACUGGACGUGGUAUCUUUC | [899-919](21/21) |
| 31 | Human | 21 | AGACGUUGAAAGAUACCACGU | ACGUGGUAUCUUUCAACGUCU | [892-912](21/21) |
| 32 | Human | 21 | CAGAUGAUGAAGCUGGAGCCA | UGGCUCCAGCUUCAUCAUCUG | [1482-1502](21/21) |
| 33 | Human | 21 | GAAGACACAGAUGAUGAAGCU | AGCUUCAUCAUCUGUGUCUUC | [1475-1495](21/21) |
| 34 | Human | 21 | GGGAAGACACAGAUGAUGAAG | CUUCAUCAUCUGUGUCUUCCC | [1473-1493](21/21) |
| 35 | Human | 21 | GAGGGAAGACACAGAUGAUGA | UCAUCAUCUGUGUCUUCCCUC | [1471-1491](21/21) |
| 36 | Human | 21 | CAGAGAGGGAAGACACAGAUG | CAUCUGUGUCUUCCCUCUCUG | [1467-1487](21/21) |
| 37 | Human | 21 | AGAGGACAGACGUUGAAAGAU | AUCUUUCAACGUCUGUCCUCU | [885-905](21/21) |
| 38 | Human | 21 | ACCAGAGGACAGACGUUGAAA | UUUCAACGUCUGUCCUCUGGU | [882-902](21/21) |
| 39 | Human | 21 | CAGAAACAGGAGCUGACAACC | GGUUGUCAGCUCCUGUUUCUG | [1371-1391](21/21) |
| 40 | Human | 21 | CAUCAGAAACAGGAGCUGACA | UGUCAGCUCCUGUUUCUGAUG | [1368-1388](21/21) |
| 41 | Human | 21 | CAUCAUCCUCACCAGUGGCAA | UUGCCACUGGUGAGGAUGAUG | [1301-1321](21/21) |
| 42 | Human | 21 | GGCAUCAUCAUCCUCACCAGU | ACUGGUGAGGAUGAUGAUGCC | [1296-1316](21/21) |
| 43 | Human | 21 | GUUCAUCACAGGCAUCAUCAU | AUGAUGAUGCCUGUGAUGAAC | [1286-1306](21/21) |
| 44 | Human | 21 | UGUUCAUCACAGGCAUCAUCA | UGAUGAUGCCUGUGAUGAACA | [1285-1305](21/21) |
| 45 | Human | 21 | GCUGUUCAUCACAGGCAUCAU | AUGAUGCCUGUGAUGAACAGC | [1283-1303](21/21) |
| 46 | Human | 21 | UGAUGAACACACCCUCCGGAA | UUCCGGAGGGUGUGUUCAUCA | [1238-1258](21/21) |
| 47 | Human | 21 | UGACCCCUUCUUCUAUGAUGA | UCAUCAUAGAAGAAGGGGUCA | [1223-1243](21/21) |
| 48 | Human | 21 | GAGGAUGACCCCUUCUUCUAU | AUAGAAGAAGGGGUCAUCCUC | [1218-1238](21/21) |
| 49 | Human | 21 | UGAGGAUGACCCCUUCUUCUA | UAGAAGAAGGGGUCAUCCUCA | [1217-1237](21/21) |
| 50 | Human | 21 | CAUGAGGAUGACCCCUUCUUC | GAAGAAGGGGUCAUCCUCAUG | [1215-1235](21/21) |
| 51 | Human | 21 | CAUCUGGUUUUCAUGAGGAUG | CAUCCUCAUGAAAACCAGAUG | [1204-1224](21/21) |
| 52 | Human | 21 | CCAUCUGGUUUUCAUGAGGAU | AUCCUCAUGAAAACCAGAUGG | [1203-1223](21/21) |
| 53 | Human | 21 | UCAAGCCAUCUGGUUUUCAUG | CAUGAAAACCAGAUGGCUUGA | [1198-1218](21/21) |
| 54 | Human | 21 | CCUCAAGCCAUCUGGUUUUCA | UGAAAACCAGAUGGCUUGAGG | [1196-1216](21/21) |
| 55 | Human | 21 | GACCCUCAAGCCAUCUGGUUU | AAACCAGAUGGCUUGAGGGUC | [1193-1213](21/21) |
| 56 | Human | 21 | CUCAUCCCACUGAUGACACCA | UGGUGUCAUCAGUGGGAUGAG | [1120-1140](21/21) |
| 57 | Human | 21 | CACCAUCGUUGGCCUGAUUCU | AGAAUCAGGCCAACGAUGGUG | [857-877](21/21) |
| 58 | Human | 21 | AAAGCAGCUCAUCCCACUGAU | AUCAGUGGGAUGAGCUGCUUU | [1113-1133](21/21) |
| 59 | Human | 21 | UCACCAUCGUUGGCCUGAUUC | GAAUCAGGCCAACGAUGGUGA | [856-876](21/21) |
| 60 | Human | 21 | AGAGCACCAAAGCAGCUCAUC | GAUGAGCUGCUUUGGUGCUCU | [1105-1125](21/21) |
| 61 | Human | 21 | ACACACAAGAGCACCAAAGCA | UGCUUUGGUGCUCUUGUGUGU | [1098-1118](21/21) |
| 62 | Human | 21 | GACACACAAGAGCACCAAAGC | GCUUUGGUGCUCUUGUGUGUC | [1097-1117](21/21) |
| 63 | Human | 21 | GAGACACAAGAGCACCAAAGC | UUUGGUGCUCUUGUGUGUCUC | [1095-1115](21/21) |
| 64 | Human | 21 | AGAUCCAGAGACACACAAGAG | CUCUUGUGUGUCUCUGGAUCU | [1088-1108](21/21) |
| 65 | Human | 21 | ACAGAUCCAGAGACACACAAG | CUUGUGUGUCUCUGGAUCUGU | [1086-1106](21/21) |
| 66 | Human | 21 | CUAGUGACAGAUCCAGAGACA | UGUCUCUGGAUCUGUCACUAG | [1080-1100](21/21) |
| 67 | Human | 21 | CUCUAGUGACAGAUCCAGAGA | UCUCUGGAUCUGUCACUAGAG | [1078-1098](21/21) |
| 68 | Human | 21 | GAUGGGCCUCUAGUGACAGAU | AUCUGUCACUAGAGGCCCAUC | [1071-1091](21/21) |
| 69 | Human | 21 | AGACCCAGCAACUGGAAGGAA | UUCCUUCCAGUUGCUGGGUCU | [1048-1068](21/21) |
| 70 | Human | 21 | GAUGCAGUCUACACAGAACUC | GAGUUCUGUGUAGACUGCAUC | [969-989](21/21) |
| 71 | Human | 21 | AGAUGCAGUCUACACAGAACU | AGUUCUGUGUAGACUGCAUCU | [968-988](21/21) |
| 72 | Human | 21 | CAACUAUCAUGGACAUUCAGG | CCUGAAUGUCCAUGAUAGUUG | [931-951](21/21) |
| 73 | Human | 21 | UCAACUAUCAUGGACAUUCAG | CUGAAUGUCCAUGAUAGUUGA | [930-950](21/21) |
| 74 | Human | 21 | AGACUCAACUAUCAUGGACAU | AUGUCCAUGAUAGUUGAGUCU | [926-946](21/21) |
| 75 | Human | 21 | CAGACUCAACUAUCAUGGACA | UGUCCAUGAUAGUUGAGUCUG | [925-945](21/21) |
| 76 | Human | 21 | GCAGACUCAACUAUCAUGGAC | GUCCAUGAUAGUUGAGUCUGC | [924-944](21/21) |
| 77 | Human | 21 | AGCAGACUCAACUAUCAUGGA | UCCAUGAUAGUUGAGUCUGCU | [923-943](21/21) |
| 78 | Human | 21 | CAGCAGACUCAACUAUCAUGG | CCAUGAUAGUUGAGUCUGCUG | [922-942](21/21) |
| 79 | Human | 21 | CUUCAGCAGACUCAACUAUCA | UGAUAGUUGAGUCUGCUGAAG | [919-939](21/21) |
| 80 | Human | 21 | UCUUCAGCAGACUCAACUAUC | GAUAGUUGAGUCUGCUGAAGA | [918-938](21/21) |
| 81 | Human | 21 | GUCCAGUUCUUCAGCAGACUC | GAGUCUGCUGAAGAACUGGAC | [911-931](21/21) |
| 82 | Human | 21 | CGUCCAGUUCUUCAGCAGACU | AGUCUGCUGAAGAACUGGACG | [910-930](21/21) |
| 83 | Human | 21 | UACCACGUCCAGUUCUUCAGC | GCUGAAGAACUGGACGUGGUA | [905-925](21/21) |
| 84 | Human | 21 | UAAAGCACUGUGGUCUUUGCC | GGCAAAGACCACAGUGCUUUA | [1611-1631](21/21) |
| 85 | Human | 21 | AAAGUAAAGCACUGUGGUCUU | AAGACCACAGUGCUUUACUUU | [1607-1627](21/21) |
| 86 | Human | 21 | AGACAAAAAGUAAAGCACUGU | ACAGUGCUUUACUUUUUGUCU | [1601-1621](21/21) |
| 87 | Human | 21 | CUAAGACAAAAAGUAAAGCAC | GUGCUUUACUUUUUGUCUUAG | [1598-1618](21/21) |
| 88 | Human | 21 | GCUAAGACAAAAAGUAAAGCA | UGCUUUACUUUUUGUCUUAGC | [1597-1617](21/21) |
| 89 | Human | 21 | CUGCUAAGACAAAAAGUAAAG | CUUUACUUUUUGUCUUAGCAG | [1595-1615](21/21) |
| 90 | Human | 21 | CCUCUGCUAAGACAAAAAGUA | UACUUUUUGUCUUAGCAGAGG | [1592-1612](21/21) |
| 91 | Human | 21 | CUCCUCUGCUAAGACAAAAAG | CUUUUUGUCUUAGCAGAGGAG | [1590-1610](21/21) |
| 92 | Human | 21 | UAUCUCCUCUGCUAAGACAAA | UUUGUCUUAGCAGAGGAGAUA | [1587-1607](21/21) |
| 93 | Human | 21 | GUCCCUCAAGUUAUCUCCUCU | AGAGGAGAUAACUUGAGGGAC | [1576-1596](21/21) |
| 94 | Human | 21 | GCUGUCCCUCAAGUUAUCUCC | GGAGAUAACUUGAGGGACAGC | [1573-1593](21/21) |
| 95 | Human | 21 | GUUGAAAGAUACCACGUCCAG | CUGGACGUGGUAUCUUUCAAC | [896-916](21/21) |
| 96 | Human | 21 | CGUUGAAAGAUACCACGUCCA | UGGACGUGGUAUCUUUCAACG | [895-915](21/21) |
| 97 | Human | 21 | ACACAGAUGAUGAAGCUGGAG | CUCCAGCUUCAUCAUCUGUGU | [1479-1499](21/21) |
| 98 | Human | 21 | GACACAGAUGAUGAAGCUGGA | UCCAGCUUCAUCAUCUGUGUC | [1478-1498](21/21) |
| 99 | Human | 21 | AAGACACAGAUGAUGAAGCUG | CAGCUUCAUCAUCUGUGUCUU | [1476-1496](21/21) |
| 100 | Human | 21 | AGAGAGGGAAGACACAGAUGA | UCAUCUGUGUCUUCCCUCUCU | [1468-1488](21/21) |

| | | | | | |
|---|---|---|---|---|---|
| | | Oligo | | mouse | |
| No | Source | Length | human 47778936 | 11612666 | rat 11612652 |

FOR 19
STRETCHES:

| | | | | | |
|---|---|---|---|---|---|
| 1 | Human | 19 | [733-751](19/19) | — | — |
| 2 | Human | 19 | [142-160](19/19) | — | — |
| 3 | Human | 19 | [458-476](19/19) | — | — |
| 4 | Human | 19 | [190-208](19/19) | — | — |
| 5 | Human | 19 | [183-201](19/19) | — | — |
| 6 | Human | 19 | [180-198](19/19) | — | — |
| 7 | Human | 19 | [178-196](19/19) | — | — |
| 8 | Human | 19 | [841-859](19/19) | — | — |
| 9 | Human | 19 | [729-747](19/19) | — | — |
| 10 | Human | 19 | [727-745](19/19) | — | — |
| 11 | Human | 19 | [147-165](19/19) | — | — |
| 12 | Human | 19 | [118-136](19/19) | — | — |
| 13 | Human | 19 | [116-134](19/19) | — | — |
| 14 | Human | 19 | [348-366](19/19) | — | — |
| 15 | Human | 19 | [346-364](19/19) | — | — |
| 16 | Human | 19 | [314-332](19/19) | — | — |
| 17 | Human | 19 | [272-290](19/19) | — | — |
| 18 | Human | 19 | [224-242](19/19) | — | — |
| 19 | Human | 19 | [176-194](19/19) | — | — |
| 20 | Human | 19 | [173-191](19/19) | — | — |
| 21 | Human | 19 | [871-889](19/19) | — | — |
| 22 | Human | 19 | [869-887](19/19) | — | — |
| 23 | Human | 19 | [162-180](19/19) | — | — |
| 24 | Human | 19 | [853-871](19/19) | — | — |
| 25 | Human | 19 | [851-869](19/19) | — | — |
| 26 | Human | 19 | [846-864](19/19) | — | — |
| 27 | Human | 19 | [831-849](19/19) | — | — |
| 28 | Human | 19 | [159-177](19/19) | — | — |
| 29 | Human | 19 | [739-757](19/19) | — | — |
| 30 | Human | 19 | [736-754](19/19) | — | — |
| 31 | Human | 19 | [148-166](19/19) | — | — |
| 32 | Human | 19 | [145-163](19/19) | — | — |
| 33 | Human | 19 | [144-162](19/19) | — | — |
| 34 | Human | 19 | [631-649](19/19) | — | — |
| 35 | Human | 19 | [597-615](19/19) | — | — |
| 36 | Human | 19 | [556-574](19/19) | — | [626-636] |
| | | | | | (11/11) |
| 37 | Human | 19 | [550-568](19/19) | — | — |
| 38 | Human | 19 | [547-565](19/19) | — | — |
| 39 | Human | 19 | [545-563](19/19) | — | — |
| 40 | Human | 19 | [535-553](19/19) | [502-515] | [605-615] |
| | | | | (14/14) | (11/11) |
| 41 | Human | 19 | [534-552](19/19) | [502-514] | [605-615] |
| | | | | (13/13) | (11/11) |
| 42 | Human | 19 | [532-550](19/19) | [502-512] | [605-615] |
| | | | | (11/11) | (11/11) |
| 43 | Human | 19 | [488-506](19/19) | — | — |
| 44 | Human | 19 | [487-505](19/19) | — | — |
| 45 | Human | 19 | [484-502](19/19) | — | — |
| 46 | Human | 19 | [481-499](19/19) | — | — |
| 47 | Human | 19 | [479-497](19/19) | — | — |
| 48 | Human | 19 | [477-495](19/19) | — | — |
| 49 | Human | 19 | [475-493](19/19) | — | — |
| 50 | Human | 19 | [462-480](19/19) | — | — |
| 51 | Human | 19 | [455-473](19/19) | — | — |
| 52 | Human | 19 | [378-396](19/19) | — | — |
| 53 | Human | 19 | [376-394](19/19) | — | — |
| 54 | Human | 19 | [374-392](19/19) | — | — |
| 55 | Human | 19 | [371-389](19/19) | — | — |
| 56 | Human | 19 | [365-383](19/19) | — | — |
| 57 | Human | 19 | [360-378](19/19) | — | — |
| 58 | Human | 19 | [357-375](19/19) | — | — |
| 59 | Human | 19 | [279-297](19/19) | — | — |
| 60 | Human | 19 | [275-293](19/19) | — | — |
| 61 | Human | 19 | [269-287](19/19) | — | — |
| 62 | Human | 19 | [104-122](19/19) | — | — |
| 63 | Human | 19 | [231-249](19/19) | — | — |
| 64 | Human | 19 | [229-247](19/19) | — | — |
| 65 | Human | 19 | [226-244](19/19) | — | — |
| 66 | Human | 19 | [193-211](19/19) | — | — |
| 67 | Human | 19 | [186-204](19/19) | — | — |
| 68 | Human | 19 | [185-203](19/19) | — | — |
| 69 | Human | 19 | [181-199](19/19) | — | — |
| 70 | Human | 19 | [172-190](19/19) | — | — |
| 71 | Human | 19 | [167-185](19/19) | — | — |
| 72 | Human | 19 | [165-183](19/19) | — | — |
| 73 | Human | 19 | [863-881](19/19) | — | — |
| 74 | Human | 19 | [859-877](19/19) | — | — |
| 75 | Human | 19 | [856-874](19/19) | — | — |
| 76 | Human | 19 | [854-872](19/19) | — | — |
| 77 | Human | 19 | [852-870](19/19) | — | — |
| 78 | Human | 19 | [850-868](19/19) | — | — |
| 79 | Human | 19 | [849-867](19/19) | — | — |
| 80 | Human | 19 | [847-865](19/19) | — | — |
| 81 | Human | 19 | [844-862](19/19) | — | — |
| 82 | Human | 19 | [161-179](19/19) | — | — |
| 83 | Human | 19 | [842-860](19/19) | — | — |
| 84 | Human | 19 | [839-857](19/19) | — | — |
| 85 | Human | 19 | [837-855](19/19) | — | — |
| 86 | Human | 19 | [834-852](19/19) | — | — |
| 87 | Human | 19 | [833-851](19/19) | — | — |
| 88 | Human | 19 | [830-848](19/19) | — | — |
| 89 | Human | 19 | [156-174](19/19) | — | — |
| 90 | Human | 19 | [152-170](19/19) | — | — |
| 91 | Human | 19 | [151-169](19/19) | — | — |
| 92 | Human | 19 | [742-760](19/19) | — | — |
| 93 | Human | 19 | [150-168](19/19) | — | — |
| 94 | Human | 19 | [732-750](19/19) | — | — |
| 95 | Human | 19 | [730-748](19/19) | — | — |
| 96 | Human | 19 | [726-744](19/19) | — | — |
| 97 | Human | 19 | [634-652](19/19) | — | — |
| 98 | Human | 19 | [630-648](19/19) | — | — |
| 99 | Human | 19 | [626-644](19/19) | — | — |
| 100 | Human | 19 | [598-616](19/19) | — | — |

FOR 21
STRETCHES:

| | | | | | |
|---|---|---|---|---|---|
| 1 | Human | 21 | [172-192](21/21) | — | — |
| 2 | Human | 21 | [534-554](21/21) | [502-515] | [605-615] |
| | | | | (14/14) | (11/11) |
| 3 | Human | 21 | [268-288](21/21) | — | — |
| 4 | Human | 21 | [225-245](21/21) | — | — |
| 5 | Human | 21 | [186-206](21/21) | — | — |
| 6 | Human | 21 | [175-195](21/21) | — | — |
| 7 | Human | 21 | [852-872](21/21) | — | — |
| 8 | Human | 21 | [840-860](21/21) | — | — |
| 9 | Human | 21 | [830-850](21/21) | — | — |
| 10 | Human | 21 | [728-748](21/21) | — | — |
| 11 | Human | 21 | [725-745](21/21) | — | — |
| 12 | Human | 21 | [147-167](21/21) | — | — |
| 13 | Human | 21 | [143-163](21/21) | — | — |
| 14 | Human | 21 | [487-507](21/21) | — | — |
| 15 | Human | 21 | [483-503](21/21) | — | — |
| 16 | Human | 21 | [480-500](21/21) | — | — |
| 17 | Human | 21 | [479-499](21/21) | — | — |
| 18 | Human | 21 | [458-478](21/21) | — | — |
| 19 | Human | 21 | [105-125](21/21) | — | — |
| 20 | Human | 21 | [179-199](21/21) | — | — |
| 21 | Human | 21 | [167-187](21/21) | — | — |
| 22 | Human | 21 | [863-883](21/21) | — | — |
| 23 | Human | 21 | [850-870](21/21) | — | — |
| 24 | Human | 21 | [161-181](21/21) | — | — |
| 25 | Human | 21 | [845-865](21/21) | — | — |
| 26 | Human | 21 | [841-861](21/21) | — | — |
| 27 | Human | 21 | [838-858](21/21) | — | — |
| 28 | Human | 21 | [833-853](21/21) | — | — |
| 29 | Human | 21 | [159-179](21/21) | — | — |
| 30 | Human | 21 | [158-178](21/21) | — | — |
| 31 | Human | 21 | [151-171](21/21) | — | — |
| 32 | Human | 21 | [741-761](21/21) | — | — |
| 33 | Human | 21 | [734-754](21/21) | — | — |
| 34 | Human | 21 | [732-752](21/21) | — | — |
| 35 | Human | 21 | [730-750](21/21) | — | — |
| 36 | Human | 21 | [726-746](21/21) | — | — |
| 37 | Human | 21 | [144-164](21/21) | — | — |
| 38 | Human | 21 | [141-161](21/21) | — | — |
| 39 | Human | 21 | [630-650](21/21) | — | — |
| 40 | Human | 21 | [627-647](21/21) | — | — |
| 41 | Human | 21 | [560-580](21/21) | — | [626-642] |
| | | | | | (16/17) |
| 42 | Human | 21 | [555-575](21/21) | — | [626-636] |
| | | | | | (11/11) |
| 43 | Human | 21 | [545-565](21/21) | — | — |
| 44 | Human | 21 | [544-564](21/21) | — | — |
| 45 | Human | 21 | [542-562](21/21) | [504-515] | — |
| | | | | (12/12) | |
| 46 | Human | 21 | [497-517](21/21) | [469-479] | [571-582] |
| | | | | (11/11) | (12/12) |
| 47 | Human | 21 | [482-502](21/21) | — | — |
| 48 | Human | 21 | [477-497](21/21) | — | — |
| 49 | Human | 21 | [476-496](21/21) | — | — |
| 50 | Human | 21 | [474-494](21/21) | — | — |
| 51 | Human | 21 | [463-483](21/21) | — | — |
| 52 | Human | 21 | [462-482](21/21) | — | — |
| 53 | Human | 21 | [457-477](21/21) | — | — |
| 54 | Human | 21 | [455-475](21/21) | — | — |
| 55 | Human | 21 | [452-472](21/21) | — | — |
| 56 | Human | 21 | [379-399](21/21) | — | — |
| 57 | Human | 21 | [116-136](21/21) | — | — |
| 58 | Human | 21 | [372-392](21/21) | — | — |
| 59 | Human | 21 | [115-135](21/21) | — | — |
| 60 | Human | 21 | [364-384](21/21) | — | — |
| 61 | Human | 21 | [357-377](21/21) | — | — |
| 62 | Human | 21 | [356-376](21/21) | — | — |
| 63 | Human | 21 | [354-374](21/21) | — | — |
| 64 | Human | 21 | [347-367](21/21) | — | — |
| 65 | Human | 21 | [345-365](21/21) | — | — |
| 66 | Human | 21 | [339-359](21/21) | — | [415-428] |
| | | | | | (14/14) |
| 67 | Human | 21 | [337-357](21/21) | — | [415-428] |
| | | | | | (14/14) |
| 68 | Human | 21 | [330-350](21/21) | — | — |
| 69 | Human | 21 | [307-327](21/21) | [275-288] | [378-391] |
| | | | | (14/14) | (14/14) |
| 70 | Human | 21 | [228-248](21/21) | — | — |
| 71 | Human | 21 | [227-247](21/21) | — | — |
| 72 | Human | 21 | [190-210](21/21) | — | — |
| 73 | Human | 21 | [189-209](21/21) | — | — |
| 74 | Human | 21 | [185-205](21/21) | — | — |
| 75 | Human | 21 | [184-204](21/21) | — | — |
| 76 | Human | 21 | [183-203](21/21) | — | — |
| 77 | Human | 21 | [182-202](21/21) | — | — |
| 78 | Human | 21 | [181-201](21/21) | — | — |
| 79 | Human | 21 | [178-198](21/21) | — | — |
| 80 | Human | 21 | [177-197](21/21) | — | — |
| 81 | Human | 21 | [170-190](21/21) | — | — |
| 82 | Human | 21 | [169-189](21/21) | — | — |
| 83 | Human | 21 | [164-184](21/21) | — | — |
| 84 | Human | 21 | [870-890](21/21) | — | — |
| 85 | Human | 21 | [866-886](21/21) | — | — |
| 86 | Human | 21 | [860-880](21/21) | — | — |
| 87 | Human | 21 | [857-877](21/21) | — | — |
| 88 | Human | 21 | [856-876](21/21) | — | — |
| 89 | Human | 21 | [854-874](21/21) | — | — |
| 90 | Human | 21 | [851-871](21/21) | — | — |
| 91 | Human | 21 | [849-869](21/21) | — | — |
| 92 | Human | 21 | [846-866](21/21) | — | — |
| 93 | Human | 21 | [835-855](21/21) | — | — |
| 94 | Human | 21 | [832-852](21/21) | — | — |
| 95 | Human | 21 | [155-175](21/21) | — | — |
| 96 | Human | 21 | [154-174](21/21) | — | — |
| 97 | Human | 21 | [738-758](21/21) | — | — |
| 98 | Human | 21 | [737-757](21/21) | — | — |
| 99 | Human | 21 | [735-755](21/21) | — | — |
| 100 | Human | 21 | [727-747](21/21) | — | — |

Table E below shows further siRNA compounds specific for the HBEGF gene that have been selected according to the present invention.

TABLE E

| | | | | | |
|---|---|---|---|---|---|
| | | Oligo | | | |
| No | Source | Length | Sense siRNA | AntiSense siRNA | human 4503412 |

FOR 19
STRETCHES:

| | | | | | |
|---|---|---|---|---|---|
| 1 | Cross Sp | 19 | CAAGUGCCUAGACUGUUAC | GUAACAGUCUAGGCACUUG | [1553-1571](19/19) |
| 2 | Cross Sp | 19 | UCAAGUGCCUAGACUGUUA | UAACAGUCUAGGCACUUGA | [1552-1570](19/19) |
| 3 | Cross Sp | 19 | UGCCUAGACUGUUACUUUG | CAAAGUAACAGUCUAGGCA | [1557-1575](19/19) |
| 4 | Cross Sp | 19 | GUGCCUAGACUGUUACUUU | AAAGUAACAGUCUAGGCAC | [1556-1574](19/19) |
| 5 | Cross Sp | 19 | AGUGCCUAGACUGUUACUU | AAGUAACAGUCUAGGCACU | [1555-1573](19/19) |
| 6 | Cross Sp | 19 | UAUUUCAAGUGCCUAGACU | AGUCUAGGCACUUGAAAUA | [1548-1566](19/19) |
| 7 | Cross Sp | 19 | UAUUGUCUGUCUACAUUUC | GAAAUGUAGACAGACAAUA | [1677-1695](19/19) |
| 8 | Cross Sp | 19 | AAGUGCCUAGACUGUUACU | AGUAACAGUCUAGGCACUU | [1554-1572](19/19) |
| 9 | Cross Sp | 19 | UUUCAAGUGCCUAGACUGU | ACAGUCUAGGCACUUGAAA | [1550-1568](19/19) |
| 10 | Cross Sp | 19 | CAAUAAUAUUUCAAGUGCC | GGCACUUGAAAUAUUAUUG | [1542-1560](19/19) |
| 11 | Cross Sp | 19 | UGCUGAAGCUCUUUCUGGC | GCCAGAAAGAGCUUCAGCA | [284-302](19/19) |
| 12 | Cross Sp | 19 | UUCAAGUGCCUAGACUGUU | AACAGUCUAGGCACUUGAA | [1551-1569](19/19) |
| 13 | Cross Sp | 19 | AUUUCAAGUGCCUAGACUG | CAGUCUAGGCACUUGAAAU | [1549-1567](19/19) |
| 14 | Cross Sp | 19 | AUAUUUCAAGUGCCUAGAC | GUCUAGGCACUUGAAAUAU | [1547-1565](19/19) |
| 15 | Cross Sp | 19 | AAUAUUUCAAGUGCCUAGA | UCUAGGCACUUGAAAUAUU | [1546-1564](19/19) |
| 16 | Cross Sp | 19 | UAAUAUUUCAAGUGCCUAG | CUAGGCACUUGAAAUAUUA | [1545-1563](19/19) |
| 17 | Cross Sp | 19 | ACCAUGAAGCUGCUGCCGU | ACGGCAGCAGCUUCAUGGU | [259-277](19/19) |
| 18 | Human | 19 | GGAUUAGAAUGCCGGUUAA | UUAACCGGCAUUCUAAUCC | [1194-1212](19/19) |
| 19 | Human | 19 | GGAAAGACUUCCAUCUAGU | ACUAGAUGGAAGUCUUUCC | [965-983](19/19) |
| 20 | Human | 19 | CCAUAGGAGAGGAGGUUAU | AUAACCUCCUCUCCUAUGG | [819-837](19/19) |
| 21 | Human | 19 | GAAGGUAUUAGCAAAGCAA | UUGCUUUGCUAAUACCUUC | [2071-2089](19/19) |
| 22 | Human | 19 | CCAAGGUGCUGAUGUCAAA | UUUGACAUCAGCACCUUGG | [1836-1854](19/19) |
| 23 | Human | 19 | CUACAUUUCUGCAGAUCUU | AAGAUCUGCAGAAAUGUAG | [1687-1705](19/19) |
| 24 | Human | 19 | CUACCCAGAUGGAAAAAUA | UAUUUUUCCAUCUGGGUAG | [1427-1445](19/19) |
| 25 | Human | 19 | AGAGCAAGAAGGAAAGAUU | AAUCUUUCCUUCUUGCUCU | [1266-1284](19/19) |
| 26 | Human | 19 | GGAUCUUUGACUGAAAAAA | UUUUUUCAGUCAAAGAUCC | [1228-1246](19/19) |
| 27 | Human | 19 | CAGGAUCUUUGACUGAAAA | UUUUCAGUCAAAGAUCCUG | [1226-1244](19/19) |
| 28 | Human | 19 | CCAGGAUCUUUGACUGAAA | UUUCAGUCAAAGAUCCUGG | [1225-1243](19/19) |
| 29 | Human | 19 | CGGUUAAAUUCCAUAUGCU | AGCAUAUGGAAUUUAACCG | [1206-1224](19/19) |
| 30 | Human | 19 | CCCCUCAUAAGUUUGUUUA | UAAACAAACUUAUGAGGGG | [1161-1179](19/19) |
| 31 | Human | 19 | CACUUCCCCUCAUAAGUUU | AAACUUAUGAGGGGAAGUG | [1156-1174](19/19) |
| 32 | Human | 19 | UAAGAAGAAAGCAAAAGCA | UGCUUUUGCUUUCUUCUUA | [1097-1115](19/19) |
| 33 | Human | 19 | CAAGUGCCAAACAGAGUAU | AUACUCUGUUUGGCACUUG | [1061-1079](19/19) |
| 34 | Human | 19 | AGAGGGGAAAGACUUCCAU | AUGGAAGUCUUUCCCCUCU | [960-978](19/19) |
| 35 | Human | 19 | GACACAAGGUGAUUUCAGA | UCUGAAAUCACCUUGUGUC | [937-955](19/19) |
| 36 | Human | 19 | GAAGACACAAGGUGAUUUC | GAAAUCACCUUGUGUCUUC | [934-952](19/19) |
| 37 | Human | 19 | CUACCUCUGAGAAGACACA | UGUGUCUUCUCAGAGGUAG | [924-942](19/19) |
| 38 | Human | 19 | GGACUGCUACCUCUGAGAA | UUCUCAGAGGUAGCAGUCC | [918-936](19/19) |
| 39 | Human | 19 | CUCAUGUUUAGGUACCAUA | UAUGGUACCUAAACAUGAG | [805-823](19/19) |
| 40 | Human | 19 | GGCUUCUCAUGUUUAGGUA | UACCUAAACAUGAGAAGCC | [800-818](19/19) |
| 41 | Human | 19 | CGUGGGGCUUCUCAUGUUU | AAACAUGAGAAGCCCCACG | [795-813](19/19) |
| 42 | Human | 19 | CUGUGGUGCUGUCAUCUGU | ACAGAUGACAGCACCACAG | [761-779](19/19) |
| 43 | Human | 19 | CCUAUGACCACACAACCAU | AUGGUUGUGUGGUCAUAGG | [728-746](19/19) |
| 44 | Human | 19 | CCAGUGGAAAAUCGCUUAU | AUAAGCGAUUUUCCACUGG | [706-724](19/19) |
| 45 | Human | 19 | GCAAAUAUGUGAAGGAGCU | AGCUCCUUCACAUAUUUGC | [623-641](19/19) |
| 46 | Human | 19 | GGAGAAUGCAAAUAUGUGA | UCACAUAUUUGCAUUCUCC | [616-634](19/19) |
| 47 | Human | 19 | CCAUGGAGAAUGCAAAUAU | AUAUUUGCAUUCUCCAUGG | [612-630](19/19) |
| 48 | Human | 19 | CAUCCAUGGAGAAUGCAAA | UUUGCAUUCUCCAUGGAUG | [609-627](19/19) |
| 49 | Human | 19 | GGAAAUACAAGGACUUCUG | CAGAAGUCCUUGUAUUUCC | [590-608](19/19) |
| 50 | Human | 19 | CCAUGUCUUCGGAAAUACA | UGUAUUUCCGAAGACAUGG | [580-598](19/19) |
| 51 | Human | 19 | GGACCCAUGUCUUCGGAAA | UUUCCGAAGACAUGGGUCC | [576-594](19/19) |
| 52 | Human | 19 | GAAGAGGGACCCAUGUCUU | AAGACAUGGGUCCCUCUUC | [570-588](19/19) |
| 53 | Human | 19 | AGAAGAGGGACCCAUGUCU | AGACAUGGGUCCCUCUUCU | [569-587](19/19) |
| 54 | Human | 19 | GGAAAAGAAAGAAGAAAGG | CCUUUCUUCUUUCUUUUCC | [536-554](19/19) |
| 55 | Human | 19 | CACGGGAAAAGAAAGAAGA | UCUUCUUUCUUUUCCCGUG | [532-550](19/19) |
| 56 | Human | 19 | CCACACCAAACAAGGAGGA | UCCUCCUUGUUUGGUGUGG | [512-530](19/19) |
| 57 | Human | 19 | CCUUUUGAGAGUCACUUUA | UAAAGUGACUCUCAAAAGG | [471-489](19/19) |
| 58 | Human | 19 | UGAACUGUUACAACUUGUA | UACAAGUUGUAACAGUUCA | [2264-2282](19/19) |
| 59 | Human | 19 | CCAUUUCUGAACUGUUACA | UGUAACAGUUCAGAAAUGG | [2257-2275](19/19) |
| 60 | Human | 19 | GGAAGUUUCUGGUGCCAUU | AAUGGCACCAGAAACUUCC | [2243-2261](19/19) |
| 61 | Human | 19 | CCAAAACGAUGAAGGUAUG | CAUACCUUCAUCGUUUUGG | [2207-2225](19/19) |
| 62 | Human | 19 | GGAAGGCUUUGUAUAAUCC | GGAUUAUACAAAGCCUUCC | [2177-2195](19/19) |
| 63 | Human | 19 | GACUGUUCUUGGUAACUGA | UCAGUUACCAAGAACAGUC | [2122-2140](19/19) |
| 64 | Human | 19 | GGAGCUGACUGUUCUUGGU | ACCAAGAACAGUCAGCUCC | [2116-2134](19/19) |
| 65 | Human | 19 | GAACAUUGGAGCUGACUGU | ACAGUCAGCUCCAAUGUUC | [2109-2127](19/19) |
| 66 | Human | 19 | GCAAAAGGCUGAGAAGGAA | UUCCUUCUCAGCCUUUUGC | [2086-2104](19/19) |
| 67 | Human | 19 | CAAAGCAAAAGGCUGAGAA | UUCUCAGCCUUUUGCUUUG | [2082-2100](19/19) |
| 68 | Human | 19 | ACAGUCUGGAGAAGGUAUU | AAUACCUUCUCCAGACUGU | [2061-2079](19/19) |
| 69 | Human | 19 | GAAAGUCCGUGACUUGCAA | UUGCAAGUCACGGACUUUC | [438-456](19/19) |
| 70 | Human | 19 | CCAUUCUUCUGGUGCUACU | AGUAGCACCAGAAGAAUGG | [2016-2034](19/19) |
| 71 | Human | 19 | CAAGACAUUUCUCUAACUC | GAGUUAGAGAAAUGUCUUG | [1994-2012](19/19) |
| 72 | Human | 19 | CUUGUGUCAAGACAUUUCU | AGAAAUGUCUUGACACAAG | [1987-2005](19/19) |
| 73 | Human | 19 | CCAUGCCUGUAGCUUUCCU | AGGAAAGCUACAGGCAUGG | [1919-1937](19/19) |
| 74 | Human | 19 | GAAGAGGUUGGGCUUCCAU | AUGGAAGCCCAACCUCUUC | [1904-1922](19/19) |
| 75 | Human | 19 | CCAAGUCUCAGAAGAGGUU | AACCUCUUCUGAGACUUGG | [1894-1912](19/19) |
| 76 | Human | 19 | CCUGCCAAGUCUCAGAAGA | UCUUCUGAGACUUGGCAGG | [1890-1908](19/19) |
| 77 | Human | 19 | GUGCUGAUGUCAAAGUGUA | UACACUUUGACAUCAGCAC | [1841-1859](19/19) |
| 78 | Human | 19 | GCGAUUUUGUCUACCAUUU | AAAUGGUAGACAAAAUCGC | [1804-1822](19/19) |
| 79 | Human | 19 | GUCUUGCCUAGGCGAUUUU | AAAAUCGCCUAGGCAAGAC | [1793-1811](19/19) |
| 80 | Human | 19 | CAGAGCCACUCUAUGAGUU | AACUCAUAGAGUGGCUCUG | [1766-1784](19/19) |
| 81 | Human | 19 | CUUGGUCAGAGCCACUCUA | UAGAGUGGCUCUGACCAAG | [1760-1778](19/19) |
| 82 | Human | 19 | CCCAUCUGUAGUAAUUUAU | AUAAAUUACUACAGAUGGG | [1661-1679](19/19) |
| 83 | Human | 19 | UGACCCAUCUGUAGUAAUU | AAUUACUACAGAUGGGUCA | [1658-1676](19/19) |
| 84 | Human | 19 | GGUUCUGUGACCCAUCUGU | ACAGAUGGGUCACAGAACC | [1651-1669](19/19) |
| 85 | Human | 19 | CCACCUUUGCCACAAAGCU | AGCUUUGUGGCAAAGGUGG | [1627-1645](19/19) |
| 86 | Human | 19 | CACUCCUCAUCCCCACAAU | AUUGUGGGGAUGAGGAGUG | [1593-1611](19/19) |
| 87 | Human | 19 | CCAGAUGGAAAAAUAACAA | UUGUUAUUUUUCCAUCUGG | [1431-1449](19/19) |
| 88 | Human | 19 | CUUCUACCCAGAUGGAAAA | UUUUCCAUCUGGGUAGAAG | [1424-1442](19/19) |
| 89 | Human | 19 | ACAAGCCUGAGAACUGAAU | AUUCAGUUCUCAGGCUUGU | [1398-1416](19/19) |
| 90 | Human | 19 | CCACAAGCCUGAGAACUGA | UCAGUUCUCAGGCUUGUGG | [1396-1414](19/19) |
| 91 | Human | 19 | CUCCAGUGCUGGAUUUGAU | AUCAAAUCCAGCACUGGAG | [1360-1378](19/19) |
| 92 | Human | 19 | GAAGCCAUGUACUCAAGUA | UACUUGAGUACAUGGCUUC | [1315-1333](19/19) |
| 93 | Human | 19 | GAAGAAAGCAACAAAGAUU | AAUCUUUGUUGCUUUCUUC | [1294-1312](19/19) |
| 94 | Human | 19 | AAAGAUUUGUGAACUGGAA | UUCCAGUUCACAAAUCUUU | [1278-1296](19/19) |
| 95 | Human | 19 | GGAAAGAUUUGUGAACUGG | CCAGUUCACAAAUCUUUCC | [1276-1294](19/19) |
| 96 | Human | 19 | GAAGGAAAGAUUUGUGAAC | GUUCACAAAUCUUUCCUUC | [1273-1291](19/19) |
| 97 | Human | 19 | AAGAAGGAAAGAUUUGUGA | UCACAAAUCUUUCCUUCUU | [1271-1289](19/19) |
| 98 | Human | 19 | CAAGAAGGAAAGAUUUGUG | CACAAAUCUUUCCUUCUUG | [1270-1288](19/19) |
| 99 | Human | 19 | GCAAGAAGGAAAGAUUUGU | ACAAAUCUUUCCUUCUUGC | [1269-1287](19/19) |
| 100 | Human | 19 | GAGCAAGAAGGAAAGAUUU | AAAUCUUUCCUUCUUGCUC | [1267-1285](19/19) |
| 101 | Human | 19 | AGAAGAAGGAGAGCAAGAA | UUCUUGCUCUCCUUCUUCU | [1257-1275](19/19) |
| 102 | Human | 19 | AGAAGAAGAAGGAGAGCAA | UUGCUCUCCUUCUUCUUCU | [1254-1272](19/19) |
| 103 | Human | 19 | GCUCCAGGAUCUUUGACUG | CAGUCAAAGAUCCUGGAGC | [1222-1240](19/19) |
| 104 | Human | 19 | CCAUAUGCUCCAGGAUCUU | AAGAUCCUGGAGCAUAUGG | [1216-1234](19/19) |
| 105 | Human | 19 | GAAUGCCGGUUAAAUUCCA | UGGAAUUUAACCGGCAUUC | [1200-1218](19/19) |
| 106 | Human | 19 | UUAGAAUGCCCGUUAAAUU | AAUUUAACCGGCAUUCUAA | [1197-1215](19/19) |
| 107 | Human | 19 | CUUAUCUUCUGGAUUAGAA | UUCUAAUCCAGAAGAUAAG | [1184-1202](19/19) |
| 108 | Human | 19 | ACACUUAUCUUCUGGAUUA | UAAUCCAGAAGAUAAGUGU | [1181-1199](19/19) |
| 109 | Human | 19 | AAACACUUAUCUUCUGGAU | AUCCAGAAGAUAAGUGUUU | [1179-1197](19/19) |
| 110 | Human | 19 | CUUCCCCUCAUAAGUUUGU | ACAAACUUAUGAGGGGAAG | [1158-1176](19/19) |
| 111 | Human | 19 | CCACUUCCCCUCAUAAGUU | AACUUAUGAGGGGAAGUGG | [1155-1173](19/19) |
| 112 | Human | 19 | CCCACUUCCCCUCAUAAGU | ACUUAUGAGGGGAAGUGGG | [1154-1172](19/19) |
| 113 | Human | 19 | AACCCCACUUCCCCUCAUA | UAUGAGGGGAAGUGGGGUU | [1151-1169](19/19) |
| 114 | Human | 19 | UGAUUCCCCUCCACCAAAC | GUUUGGUGGAGGGGAAUCA | [1135-1153](19/19) |
| 115 | Human | 19 | CCUUCAUGCCCUUCUGAUU | AAUCAGAAGGGCAUGAAGG | [1121-1139](19/19) |
| 116 | Human | 19 | GACCUUCAUGCCCUUCUGA | UCAGAAGGGCAUGAAGGUC | [1119-1137](19/19) |

FOR 21
STRETCHES:

| | | | | | |
|---|---|---|---|---|---|
| 1 | Cross Sp | 21 | UCAAGUGCCUAGACUGUUACU | AGUAACAGUCUAGGCACUUGA | [1552-1572](21/21) |
| 2 | Cross Sp | 21 | CAAGUGCCUAGACUGUUACUU | AAGUAACAGUCUAGGCACUUG | [1553-1573](21/21) |
| 3 | Cross Sp | 21 | AAGUGCCUAGACUGUUACUUU | AAAGUAACAGUCUAGGCACUU | [1554-1574](21/21) |
| 4 | Cross Sp | 21 | AUAUUUCAAGUGCCUAGACUG | CAGUCUAGGCACUUGAAAUAU | [1547-1567](21/21) |
| 5 | Cross Sp | 21 | UUCAAGUGCCUAGACUGUUAC | GUAACAGUCUAGGCACUUGAA | [1551-1571](21/21) |
| 6 | Cross Sp | 21 | AUUUCAAGUGCCUAGACUGUU | AACAGUCUAGGCACUUGAAAU | [1549-1569](21/21) |
| 7 | Cross Sp | 21 | UAAUAUUUCAAGUGCCUAGAC | GUCUAGGCACUUGAAAUAUUA | [1545-1565](21/21) |
| 8 | Cross Sp | 21 | AGUGCCUAGACUGUUACUUUG | CAAAGUAACAGUCUAGGCACU | [1555-1575](21/21) |
| 9 | Cross Sp | 21 | UUUCAAGUGCCUAGACUGUUA | UAACAGUCUAGGCACUUGAAA | [1550-1570](21/21) |
| 10 | Cross Sp | 21 | UAUUUCAAGUGCCUAGACUGU | ACAGUCUAGGCACUUGAAAUA | [1548-1568](21/21) |
| 11 | Cross Sp | 21 | AAUAUUUCAAGUGCCUAGACU | AGUCUAGGCACUUGAAAUAUU | [1546-1566](21/21) |
| 12 | Human | 21 | CAAGAAGGAAAGAUUUGUGAA | UUCACAAAUCUUUCCUUCUUG | [1270-1290](21/21) |
| 13 | Human | 21 | CCAGGAUCUUUGACUGAAAAA | UUUUUCAGUCAAAGAUCCUGG | [1225-1245](21/21) |
| 14 | Human | 21 | CCAUGGAGAAUGCAAAUAUGU | ACAUAUUUGCAUUCUCCAUGG | [612-632](21/21) |
| 15 | Human | 21 | GCAUCCAUGGAGAAUGCAAAU | AUUUGCAUUCUCCAUGGAUGC | [608-628](21/21) |
| 16 | Human | 21 | GCACGGGAAAAGAAAGAAGAA | UUCUUCUUUCUUUUCCCGUGC | [531-551](21/21) |
| 17 | Human | 21 | GCAGAUCUGGACCUUUUGAGA | UCUCAAAAGGUCCAGAUCUGC | [460-480](21/21) |
| 18 | Human | 21 | GGAACAUUGGAGCUGACUGUU | AACAGUCAGCUCCAAUGUUCC | [2108-2128](21/21) |
| 19 | Human | 21 | GAAGAAAGCAACAAAGAUUGA | UCAAUCUUUGUUGCUUUCUUC | [1294-1314](21/21) |
| 20 | Human | 21 | GCAAGAAGGAAAGAUUUGUGA | UCACAAAUCUUUCCUUCUUGC | [1269-1289](21/21) |
| 21 | Human | 21 | GAGAGCAAGAAGGAAAGAUUU | AAAUCUUUCCUUCUUGCUCUC | [1265-1285](21/21) |
| 22 | Human | 21 | GAAGAAGGAGAGCAAGAAGGA | UCCUUCUUGCUCUCCUUCUUC | [1258-1278](21/21) |
| 23 | Human | 21 | CCACUUCCCCUCAUAAGUUUG | CAAACUUAUGAGGGGAAGUGG | [1155-1175](21/21) |
| 24 | Human | 21 | AAACCCCACUUCCCCUCAUAA | UUAUGAGGGGAAGUGGGGUUU | [1150-1170](21/21) |
| 25 | Human | 21 | GGACCUUCAUGCCCUUCUGAU | AUCAGAAGGGCAUGAAGGUCC | [1118-1138](21/21) |
| 26 | Human | 21 | GCAAAAGCAAGGGACCUUCAU | AUGAAGGUCCCUUGCUUUUGC | [1107-1127](21/21) |
| 27 | Human | 21 | GUAAGAAGAAAGCAAAAGCAA | UUGCUUUUGCUUUCUUCUUAC | [1096-1116](21/21) |
| 28 | Human | 21 | CUGGGUAAGAAGAAAGCAAAA | UUUUGCUUUCUUCUUACCCAG | [1092-1112](21/21) |
| 29 | Human | 21 | CAAGUGCCAAACAGAGUAUGU | ACAUACUCUGUUUGGCACUUG | [1061-1081](21/21) |
| 30 | Human | 21 | CCAAAAUACCAGAGCCUUCAA | UUGAAGGCUCUGGUAUUUUGG | [1043-1063](21/21) |
| 31 | Human | 21 | GGAUUGGGCCUCCCAUAAUUG | CAAUUAUGGGAGGCCCAAUCC | [1017-1037](21/21) |
| 32 | Human | 21 | CUAGGAUUGGGCCUCCCAUAA | UUAUGGGAGGCCCAAUCCUAG | [1014-1034](21/21) |
| 33 | Human | 21 | GGAAAGACUUCCAUCUAGUCA | UGACUAGAUGGAAGUCUUUCC | [965-985](21/21) |
| 34 | Human | 21 | GAGGGGAAAGACUUCCAUCUA | UAGAUGGAAGUCUUUCCCCUC | [961-981](21/21) |
| 35 | Human | 21 | GCAGAGGGGAAAGACUUCCAU | AUGGAAGUCUUUCCCCUCUGC | [958-978](21/21) |
| 36 | Human | 21 | AGAAGACACAAGGUGAUUUCA | UGAAAUCACCUUGUGUCUUCU | [933-953](21/21) |
| 37 | Human | 21 | CUGAGAAGACACAAGGUGAUU | AAUCACCUUGUGUCUUCUCAG | [930-950](21/21) |
| 38 | Human | 21 | GCUACCUCUGAGAAGACACAA | UUGUGUCUUCUCAGAGGUAGC | [923-943](21/21) |
| 39 | Human | 21 | GAGAGACUUGUGCUCAAGGAA | UUCCUUGAGCACAAGUCUCUC | [889-909](21/21) |
| 40 | Human | 21 | GGUUAUGAUGUGGAAAAUGAA | UUCAUUUUCCACAUCAUAACC | [832-852](21/21) |
| 41 | Human | 21 | GGAGGUUAUGAUGUGGAAAAU | AUUUUCCACAUCAUAACCUCC | [829-849](21/21) |
| 42 | Human | 21 | GAGGAGGUUAUGAUGUGGAAA | UUUCCACAUCAUAACCUCCUC | [827-847](21/21) |
| 43 | Human | 21 | GUACCAUAGGAGAGGAGGUUA | UAACCUCCUCUCCUAUGGUAC | [816-836](21/21) |
| 44 | Human | 21 | GGAAAAUCGCUUAUAUACCUA | UAGGUAUAUAAGCGAUUUUCC | [711-731](21/21) |
| 45 | Human | 21 | CAGUGGAAAAUCGCUUAUAUA | UAUAUAAGCGAUUUUCCACUG | [707-727](21/21) |
| 46 | Human | 21 | GGGACCCAUGUCUUCGGAAAU | AUUUCCGAAGACAUGGGUCCC | [575-595](21/21) |
| 47 | Human | 21 | GGAGCACGGGAAAAGAAAGAA | UUCUUUCUUUUCCCGUGCUCC | [528-548](21/21) |
| 48 | Human | 21 | GGAGGAGCACGGGAAAAGAAA | UUUCUUUUCCCGUGCUCCUCC | [525-545](21/21) |
| 49 | Human | 21 | GGACCUUUUGAGAGUCACUUU | AAAGUGACUCUCAAAAGGUCC | [468-488](21/21) |
| 50 | Human | 21 | CCAAACCUGGUUCAUAUUUAU | AUAAAUAUGAACCAGGUUUGG | [2286-2306](21/21) |
| 51 | Human | 21 | GUAUUUCCAAACCUGGUUCAU | AUGAACCAGGUUUGGAAAUAC | [2280-2300](21/21) |
| 52 | Human | 21 | CUGAACUGUUACAACUUGUAU | AUACAAGUUGUAACAGUUCAG | [2263-2283](21/21) |
| 53 | Human | 21 | CCACCUCACCAAAACGAUGAA | UUCAUCGUUUUGGUGAGGUGG | [2199-2219](21/21) |
| 54 | Human | 21 | CACCCACCUCACCAAAACGAU | AUCGUUUUGGUGAGGUGGGUG | [2196-2216](21/21) |
| 55 | Human | 21 | GAGGUGGGGAAGGCUUUGUAU | AUACAAAGCCUUCCCCACCUC | [2170-2190](21/21) |
| 56 | Human | 21 | CCAAUUGCUACCGAGAAGGUU | AACCUUCUCGGUAGCAAUUGG | [2148-2168](21/21) |
| 57 | Human | 21 | GUAACUGAUUACCUGCCAAUU | AAUUGGCAGGUAAUCAGUUAC | [2133-2153](21/21) |
| 58 | Human | 21 | UGACUGUUCUUGGUAACUGAU | AUCAGUUACCAAGAACAGUCA | [2121-2141](21/21) |
| 59 | Human | 21 | ACAUUGGAGCUGACUGUUCUU | AAGAACAGUCAGCUCCAAUGU | [2111-2131](21/21) |
| 60 | Human | 21 | GGAACAGGGAACAUUGGAGCU | AGCUCCAAUGUUCCCUGUUCC | [2101-2121](21/21) |
| 61 | Human | 21 | GAAGGAACAGGGAACAUUGGA | UCCAAUGUUCCCUGUUCCUUC | [2098-2118](21/21) |
| 62 | Human | 21 | GCAAAGCAAAAGGCUGAGAAG | CUUCUCAGCCUUUUGCUUUGC | [2081-2101](21/21) |
| 63 | Human | 21 | GAAGGUAUUAGCAAAGCAAAA | UUUUGCUUUGCUAAUACCUUC | [2071-2091](21/21) |
| 64 | Human | 21 | AGAAGGUAUUAGCAAAGCAAA | UUUGCUUUGCUAAUACCUUCU | [2070-2090](21/21) |
| 65 | Human | 21 | GACAGUCUGGAGAAGGUAUUA | UAAUACCUUCUCCAGACUGUC | [2060-2080](21/21) |
| 66 | Human | 21 | GAGGACAGUCUGGAGAAGGUA | UACCUUCUCCAGACUGUCCUC | [2057-2077](21/21) |
| 67 | Human | 21 | GGAAAGUCCGUGACUUGCAAG | CUUGCAAGUCACGGACUUUCC | [437-457](21/21) |
| 68 | Human | 21 | GAACUCACUUUCCCUUGUGUC | GACACAAGGGAAAGUGAGUUC | [1974-1994](21/21) |
| 69 | Human | 21 | GGAACUCACUUUCCCUUGUGU | ACACAAGGGAAAGUGAGUUCC | [1973-1993](21/21) |
| 70 | Human | 21 | GAUGUCAAAGUGUAACAGAUA | UAUCUGUUACACUUUGACAUC | [1846-1866](21/21) |
| 71 | Human | 21 | GGUGCUGAUGUCAAAGUGUAA | UUACACUUUGACAUCAGCACC | [1840-1860](21/21) |
| 72 | Human | 21 | CUACCAUUUGUGUUUUGAAAG | CUUUCAAAACACAAAUGGUAG | [1814-1834](21/21) |
| 73 | Human | 21 | CUAGGCGAUUUUGUCUACCAU | AUGGUAGACAAAAUCGCCUAG | [1800-1820](21/21) |
| 74 | Human | 21 | CUCUAUGAGUUGGACUUCAGU | ACUGAAGUCCAACUCAUAGAG | [1774-1794](21/21) |
| 75 | Human | 21 | CCACUCUAUGAGUUGGACUUC | GAAGUCCAACUCAUAGAGUGG | [1771-1791](21/21) |
| 76 | Human | 21 | GGUCAGAGCCACUCUAUGAGU | ACUCAUAGAGUGGCUCUGACC | [1763-1783](21/21) |
| 77 | Human | 21 | GUAGGGGUUAACUUGGUCAGA | UCUGACCAAGUUAACCCCUAC | [1749-1769](21/21) |
| 78 | Human | 21 | GUGACCCAUCUGUAGUAAUUU | AAAUUACUACAGAUGGGUCAC | [1657-1677](21/21) |
| 79 | Human | 21 | CCACAAAGCUAGGAUGGUUCU | AGAACCAUCCUAGCUUUGUGG | [1636-1656](21/21) |
| 80 | Human | 21 | CUAGACUGUUACUUUGGCAAU | AUUGCCAAAGUAACAGUCUAG | [1560-1580](21/21) |
| 81 | Human | 21 | CCAGAUGGAAAAAUAACAACU | AGUUGUUAUUUUUCCAUCUGG | [1431-1451](21/21) |
| 82 | Human | 21 | CAAGCCUGAGAACUGAAUUUU | AAAAUUCAGUUCUCAGGCUUG | [1399-1419](21/21) |
| 83 | Human | 21 | CACAAGCCUGAGAACUGAAUU | AAUUCAGUUCUCAGGCUUGUG | [1397-1417](21/21) |
| 84 | Human | 21 | GGAUUUGAUGAGUUAACUGUG | CACAGUUAACUCAUCAAAUCC | [1370-1390](21/21) |
| 85 | Human | 21 | GUGCUGGAUUUGAUGAGUUAA | UUAACUCAUCAAAUCCAGCAC | [1365-1385](21/21) |
| 86 | Human | 21 | CCAGUGCUGGAUUUGAUGAGU | ACUCAUCAAAUCCAGCACUGG | [1362-1382](21/21) |
| 87 | Human | 21 | GAAGCCAUGUACUCAAGUACC | GGUACUUGAGUACAUGGCUUC | [1315-1335](21/21) |
| 88 | Human | 21 | UGAGAAGCCAUGUACUCAAGU | ACUUGAGUACAUGGCUUCUCA | [1312-1332](21/21) |
| 89 | Human | 21 | AGAUUUGUGAACUGGAAGAAA | UUUCUUCCAGUUCACAAAUCU | [1280-1300](21/21) |
| 90 | Human | 21 | GAAAGAUUUGUGAACUGGAAG | CUUCCAGUUCACAAAUCUUUC | [1277-1297](21/21) |
| 91 | Human | 21 | AGAAGGAAAGAUUUGUGAACU | AGUUCACAAAUCUUUCCUUCU | [1272-1292](21/21) |
| 92 | Human | 21 | GGAGAGCAAGAAGGAAAGAUU | AAUCUUUCCUUCUUGCUCUCC | [1264-1284](21/21) |
| 93 | Human | 21 | AAGGAGAGCAAGAAGGAAAGA | UCUUUCCUUCUUGCUCUCCUU | [1262-1282](21/21) |
| 94 | Human | 21 | GAAGGAGAGCAAGAAGGAAAG | CUUUCCUUCUUGCUCUCCUUC | [1261-1281](21/21) |
| 95 | Human | 21 | GAAGAAGAAGGAGAGCAAGAA | UUCUUGCUCUCCUUCUUCUUC | [1255-1275](21/21) |
| 96 | Human | 21 | CAUAUGCUCCAGGAUCUUUGA | UCAAAGAUCCUGGAGCAUAUG | [1217-1237](21/21) |
| 97 | Human | 21 | UUCCAUAUGCUCCAGGAUCUU | AAGAUCCUGGAGCAUAUGGAA | [1214-1234](21/21) |
| 98 | Human | 21 | CCGGUUAAAUUCCAUAUGCUC | GAGCAUAUGGAAUUUAACCGG | [1205-1225](21/21) |
| 99 | Human | 21 | GCCGGUUAAAUUCCAUAUGCU | AGCAUAUGGAAUUUAACCGGC | [1204-1224](21/21) |
| 100 | Human | 21 | GAAUGCCGGUUAAAUUCCAUA | UAUGGAAUUUAACCGGCAUUC | [1200-1220](21/21) |
| 101 | Human | 21 | AGAAUGCCGGUUAAAUUCCAU | AUGGAAUUUAACCGGCAUUCU | [1199-1219](21/21) |
| 102 | Human | 21 | GGAUUAGAAUGCCGGUUAAAU | AUUUAACCGGCAUUCUAAUCC | [1194-1214](21/21) |
| 103 | Human | 21 | UGGAUUAGAAUGCCGGUUAAA | UUUAACCGGCAUUCUAAUCCA | [1193-1213](21/21) |
| 104 | Human | 21 | CUGGAUUAGAAUGCCGGUUAA | UUAACCGGCAUUCUAAUCCAG | [1192-1212](21/21) |
| 105 | Human | 21 | CUUCUGGAUUAGAAUGCCGGU | ACCGGCAUUCUAAUCCAGAAG | [1189-1209](21/21) |
| 106 | Human | 21 | ACACUUAUCUUCUGGAUUAGA | UCUAAUCCAGAAGAUAAGUGU | [1181-1201](21/21) |
| 107 | Human | 21 | UCCCCUCAUAAGUUUGUUUAA | UUAAACAAACUUAUGAGGGGA | [1160-1180](21/21) |
| 108 | Human | 21 | CCCCACUUCCCCUCAUAAGUU | AACUUAUGAGGGGAAGUGGGG | [1153-1173](21/21) |
| 109 | Human | 21 | CAAACCCCACUUCCCCUCAUA | UAUGAGGGGAAGUGGGGUUUG | [1149-1169](21/21) |
| 110 | Human | 21 | GACCUUCAUGCCCUUCUGAUU | AAUCAGAAGGGCAUGAAGGUC | [1119-1139](21/21) |

| | | | | |
|---|---|---|---|---|
| | | Oligo | | |
| No | Source | Length | mouse 6754177 | rat 6978784 |

FOR 19
STRETCHES:

| | | | | |
|---|---|---|---|---|
| 1 | Cross Sp | 19 | [1570-1588](19/19) | [1324-1342](19/19) |
| 2 | Cross Sp | 19 | [1569-1587](19/19) | [1323-1341](19/19) |
| 3 | Cross Sp | 19 | [1574-1592](19/19) | [1328-1346](19/19) |
| 4 | Cross Sp | 19 | [1573-1591](19/19) | [1327-1345](19/19) |
| 5 | Cross Sp | 19 | [1572-1590](19/19) | [1326-1344](19/19) |
| 6 | Cross Sp | 19 | [1565-1583](19/19) | [1319-1337](19/19) |
| 7 | Cross Sp | 19 | [1683-1701](19/19) | [1434-1452](19/19) |
| 8 | Cross Sp | 19 | [1571-1589](19/19) | [1325-1343](19/19) |
| 9 | Cross Sp | 19 | [1567-1585](19/19) | [1321-1339](19/19) |
| 10 | Cross Sp | 19 | [1559-1577](19/19) | [1313-1331](19/19) |
| 11 | Cross Sp | 19 | [284-302](19/19) | [54-72](19/19) |
| 12 | Cross Sp | 19 | [1568-1586](19/19) | [1322-1340](19/19) |
| 13 | Cross Sp | 19 | [1566-1584](19/19) | [1320-1338](19/19) |
| 14 | Cross Sp | 19 | [1564-1582](19/19) | [1318-1336](19/19) |
| 15 | Cross Sp | 19 | [1563-1581](19/19) | [1317-1335](19/19) |
| 16 | Cross Sp | 19 | [1562-1580](19/19) | [1316-1334](19/19) |
| 17 | Cross Sp | 19 | [259-277](19/19) | [29-47](19/19) |
| 18 | Human | 19 | — | — |
| 19 | Human | 19 | — | — |
| 20 | Human | 19 | [823-837](15/15) | [589-607](18/19) |
| 21 | Human | 19 | — | — |
| 22 | Human | 19 | [1843-1857](15/15) | — |
| 23 | Human | 19 | — | — |
| 24 | Human | 19 | — | — |
| 25 | Human | 19 | — | — |
| 26 | Human | 19 | — | — |
| 27 | Human | 19 | — | — |
| 28 | Human | 19 | — | — |
| 29 | Human | 19 | [1216-1226](11/11) | [979-989](11/11) |
| 30 | Human | 19 | — | — |
| 31 | Human | 19 | — | — |
| 32 | Human | 19 | [1110-1120](11/11) | [873-883](11/11) |
| 33 | Human | 19 | [1074-1087](14/14) | [835-848](14/14) |
| 34 | Human | 19 | — | — |
| 35 | Human | 19 | — | — |
| 36 | Human | 19 | — | — |
| 37 | Human | 19 | — | — |
| 38 | Human | 19 | — | — |
| 39 | Human | 19 | [805-821](17/17) | — |
| 40 | Human | 19 | [802-818](17/17) | [572-588](16/17) |
| 41 | Human | 19 | [795-813](18/19) | [565-582](17/18) |
| 42 | Human | 19 | — | [539-549](11/11) |
| 43 | Human | 19 | [730-740](11/11) | [500-510](11/11) |
| 44 | Human | 19 | — | — |
| 45 | Human | 19 | — | — |
| 46 | Human | 19 | — | — |
| 47 | Human | 19 | — | — |
| 48 | Human | 19 | — | — |
| 49 | Human | 19 | [590-604](15/15) | [361-374](14/14) |
| 50 | Human | 19 | — | — |
| 51 | Human | 19 | — | — |
| 52 | Human | 19 | — | — |
| 53 | Human | 19 | — | — |
| 54 | Human | 19 | [544-554](11/11) | — |
| 55 | Human | 19 | — | — |
| 56 | Human | 19 | — | — |
| 57 | Human | 19 | — | — |
| 58 | Human | 19 | [2259-2274](16/16) | — |
| 59 | Human | 19 | [2252-2270](18/19) | — |
| 60 | Human | 19 | — | — |
| 61 | Human | 19 | — | — |
| 62 | Human | 19 | — | — |
| 63 | Human | 19 | — | — |
| 64 | Human | 19 | — | — |
| 65 | Human | 19 | — | — |
| 66 | Human | 19 | [2083-2093](11/11) | — |
| 67 | Human | 19 | [2078-2093](15/16) | — |
| 68 | Human | 19 | — | — |
| 69 | Human | 19 | — | — |
| 70 | Human | 19 | — | — |
| 71 | Human | 19 | [1990-2000](11/11) | — |
| 72 | Human | 19 | [1984-2000](17/17) | — |
| 73 | Human | 19 | — | — |
| 74 | Human | 19 | — | — |
| 75 | Human | 19 | — | — |
| 76 | Human | 19 | — | — |
| 77 | Human | 19 | — | — |
| 78 | Human | 19 | [1815-1825](11/11) | — |
| 79 | Human | 19 | [1799-1817](18/19) | — |
| 80 | Human | 19 | — | [1523-1541](18/19) |
| 81 | Human | 19 | — | [1518-1534](16/17) |
| 82 | Human | 19 | [1667-1685](18/19) | [1418-1436](19/19) |
| 83 | Human | 19 | — | [1417-1433](17/17) |
| 84 | Human | 19 | — | — |
| 85 | Human | 19 | — | — |
| 86 | Human | 19 | — | — |
| 87 | Human | 19 | — | [1191-1209](18/19) |
| 88 | Human | 19 | [1425-1435](11/11) | [1184-1202](18/19) |
| 89 | Human | 19 | — | — |
| 90 | Human | 19 | — | — |
| 91 | Human | 19 | [1362-1373](12/12) | [1124-1135](12/12) |
| 92 | Human | 19 | — | — |
| 93 | Human | 19 | — | — |
| 94 | Human | 19 | — | — |
| 95 | Human | 19 | — | — |
| 96 | Human | 19 | — | — |
| 97 | Human | 19 | — | — |
| 98 | Human | 19 | — | — |
| 99 | Human | 19 | — | — |
| 100 | Human | 19 | — | — |
| 101 | Human | 19 | — | — |
| 102 | Human | 19 | — | — |
| 103 | Human | 19 | — | — |
| 104 | Human | 19 | [1218-1233](16/16) | [981-996](16/16) |
| 105 | Human | 19 | — | — |
| 106 | Human | 19 | — | — |
| 107 | Human | 19 | [1186-1203](17/18) | [949-964](15/16) |
| 108 | Human | 19 | [1183-1201](18/19) | [946-964](18/19) |
| 109 | Human | 19 | — | — |
| 110 | Human | 19 | — | — |
| 111 | Human | 19 | — | — |
| 112 | Human | 19 | — | — |
| 113 | Human | 19 | — | — |
| 114 | Human | 19 | — | — |
| 115 | Human | 19 | — | — |
| 116 | Human | 19 | — | — |

FOR 21
STRETCHES:

| | | | | |
|---|---|---|---|---|
| 1 | Cross Sp | 21 | [1569-1589](21/21) | [1323-1343](21/21) |
| 2 | Cross Sp | 21 | [1570-1590](21/21) | [1324-1344](21/21) |
| 3 | Cross Sp | 21 | [1571-1591](21/21) | [1325-1345](21/21) |
| 4 | Cross Sp | 21 | [1564-1584](21/21) | [1318-1338](21/21) |
| 5 | Cross Sp | 21 | [1568-1588](21/21) | [1322-1342](21/21) |
| 6 | Cross Sp | 21 | [1566-1586](21/21) | [1320-1340](21/21) |
| 7 | Cross Sp | 21 | [1562-1582](21/21) | [1316-1336](21/21) |
| 8 | Cross Sp | 21 | [1572-1592](21/21) | [1326-1346](21/21) |
| 9 | Cross Sp | 21 | [1567-1587](21/21) | [1321-1341](21/21) |
| 10 | Cross Sp | 21 | [1565-1585](21/21) | [1319-1339](21/21) |
| 11 | Cross Sp | 21 | [1563-1583](21/21) | [1317-1337](21/21) |
| 12 | Human | 21 | — | — |
| 13 | Human | 21 | — | — |
| 14 | Human | 21 | — | — |
| 15 | Human | 21 | — | — |
| 16 | Human | 21 | — | — |
| 17 | Human | 21 | — | [233-246](14/14) |
| 18 | Human | 21 | — | — |
| 19 | Human | 21 | — | — |
| 20 | Human | 21 | — | — |
| 21 | Human | 21 | — | — |
| 22 | Human | 21 | — | — |
| 23 | Human | 21 | — | — |
| 24 | Human | 21 | — | — |
| 25 | Human | 21 | — | — |
| 26 | Human | 21 | — | — |
| 27 | Human | 21 | [1110-1120](11/11) | [873-883](11/11) |
| 28 | Human | 21 | [1110-1120](11/11) | [873-883](11/11) |
| 29 | Human | 21 | [1074-1087](14/14) | [835-853](18/19) |
| 30 | Human | 21 | — | — |
| 31 | Human | 21 | — | — |
| 32 | Human | 21 | — | — |
| 33 | Human | 21 | — | — |
| 34 | Human | 21 | — | — |
| 35 | Human | 21 | — | — |
| 36 | Human | 21 | — | — |
| 37 | Human | 21 | — | — |
| 38 | Human | 21 | — | — |
| 39 | Human | 21 | — | — |
| 40 | Human | 21 | — | — |
| 41 | Human | 21 | [829-839](11/11) | [599-609](11/11) |
| 42 | Human | 21 | [827-839](13/13) | [597-609](13/13) |
| 43 | Human | 21 | [816-836](20/21) | — |
| 44 | Human | 21 | — | — |
| 45 | Human | 21 | — | — |
| 46 | Human | 21 | — | — |
| 47 | Human | 21 | — | — |
| 48 | Human | 21 | — | — |
| 49 | Human | 21 | — | — |
| 50 | Human | 21 | [2284-2302](19/19) | — |
| 51 | Human | 21 | [2284-2296](13/13) | — |
| 52 | Human | 21 | [2258-2274](17/17) | — |
| 53 | Human | 21 | — | — |
| 54 | Human | 21 | — | — |
| 55 | Human | 21 | — | — |
| 56 | Human | 21 | — | — |
| 57 | Human | 21 | — | — |
| 58 | Human | 21 | — | — |
| 59 | Human | 21 | — | — |
| 60 | Human | 21 | — | — |
| 61 | Human | 21 | — | — |
| 62 | Human | 21 | [2078-2093](15/16) | — |
| 63 | Human | 21 | — | — |
| 64 | Human | 21 | — | — |
| 65 | Human | 21 | — | — |
| 66 | Human | 21 | — | — |
| 67 | Human | 21 | — | — |
| 68 | Human | 21 | — | — |
| 69 | Human | 21 | — | — |
| 70 | Human | 21 | — | — |
| 71 | Human | 21 | [1847-1857](11/11) | — |
| 72 | Human | 21 | — | — |
| 73 | Human | 21 | [1815-1825](11/11) | — |
| 74 | Human | 21 | [1780-1800](19/21) | [1531-1546](15/16) |
| 75 | Human | 21 | [1777-1795](18/19) | [1528-1546](18/19) |
| 76 | Human | 21 | — | [1523-1540](17/18) |
| 77 | Human | 21 | — | — |
| 78 | Human | 21 | — | [1417-1434](18/18) |
| 79 | Human | 21 | — | — |
| 80 | Human | 21 | [1577-1597](21/21) | [1331-1351](20/21) |
| 81 | Human | 21 | — | [1191-1209](18/19) |
| 82 | Human | 21 | — | — |
| 83 | Human | 21 | — | — |
| 84 | Human | 21 | [1365-1385](19/21) | [1127-1147](20/21) |
| 85 | Human | 21 | [1362-1375](14/14) | [1124-1138](15/15) |
| 86 | Human | 21 | [1362-1375](14/14) | [1124-1138](15/15) |
| 87 | Human | 21 | — | — |
| 88 | Human | 21 | — | — |
| 89 | Human | 21 | — | — |
| 90 | Human | 21 | — | — |
| 91 | Human | 21 | — | — |
| 92 | Human | 21 | — | — |
| 93 | Human | 21 | — | — |
| 94 | Human | 21 | — | — |
| 95 | Human | 21 | — | — |
| 96 | Human | 21 | [1219-1239](20/21) | [982-1001](19/20) |
| 97 | Human | 21 | [1216-1233](18/18) | [979-996](18/18) |
| 98 | Human | 21 | [1216-1227](12/12) | [979-990](12/12) |
| 99 | Human | 21 | [1216-1226](11/11) | [979-989](11/11) |
| 100 | Human | 21 | — | — |
| 101 | Human | 21 | — | — |
| 102 | Human | 21 | — | — |
| 103 | Human | 21 | — | — |
| 104 | Human | 21 | — | — |
| 105 | Human | 21 | [1191-1203](13/13) | [954-964](11/11) |
| 106 | Human | 21 | [1183-1203](20/21) | [946-964](18/19) |
| 107 | Human | 21 | — | — |
| 108 | Human | 21 | — | — |
| 109 | Human | 21 | — | — |
| 110 | Human | 21 | — | — |

Table F below shows further siRNA compounds specific for the IQGAP1 gene that have been selected according to the present invention.

TABLE F

| | | | | | |
|---|---|---|---|---|---|
| | | Oligo | | | |
| No | Source | Length | Sense siRNA | AntiSense siRNA | human 57242794 |

FOR 19
STRETCHES:

| | | | | | |
|---|---|---|---|---|---|
| 1 | Cross Sp | 19 | GGAUGAGUCAGCUGUGUUA | UAACACAGCUGACUCAUCC | [1886-1904](19/19) |
| 2 | Cross Sp | 19 | CCAUCUUACUGAAUACAAA | UUUGUAUUCAGUAAGAUGG | [4249-4267](19/19) |
| 3 | Cross Sp | 19 | CUACAAGACUCUCAUCAAU | AUUGAUGAGAGUCUUGUAG | [2663-2681](19/19) |
| 4 | Cross Sp | 19 | GAAUCUGGCAGUCCAACAA | UUGUUGGACUGCCAGAUUC | [1927-1945](19/19) |
| 5 | Cross Sp | 19 | UGAAUGCCAUGGAUGAGAU | AUCUCAUCCAUGGCAUUCA | [460-478](19/19) |
| 6 | Cross Sp | 19 | GGAUGAAAUUCAAGGUGGA | UCCACCUUGAAUUUCAUCC | [1910-1928](19/19) |
| 7 | Cross Sp | 19 | GAUCUUCCUUCUCAACAAA | UUUGUUGAGAAGGAAGAUC | [5039-5057](19/19) |
| 8 | Cross Sp | 19 | GCAAAAAGAUUUCUCUGAA | UUCAGAGAAAUCUUUUUGC | [4768-4786](19/19) |
| 9 | Cross Sp | 19 | GAGAGACCUUGACUGAAAU | AUUUCAGUCAAGGUCUCUC | [4303-4321](19/19) |
| 10 | Cross Sp | 19 | GGAGAGACCUUGACUGAAA | UUUCAGUCAAGGUCUCUCC | [4302-4320](19/19) |
| 11 | Cross Sp | 19 | CAGAAAUUCAGACGGUUUU | AAAACCGUCUGAAUUUCUG | [3879-3897](19/19) |
| 12 | Cross Sp | 19 | UCAGCUCUGUGGACAAAAU | AUUUUGUCCACAGAGCUGA | [3550-3568](19/19) |
| 13 | Cross Sp | 19 | GACUACAAGACUCUCAUCA | UGAUGAGAGUCUUGUAGUC | [2661-2679](19/19) |
| 14 | Cross Sp | 19 | GAAGAAAUCAACAACAUGA | UCAUGUUGUUGAUUUCUUC | [639-657](19/19) |
| 15 | Cross Sp | 19 | UCAGUUUGUACCUGUUCAA | UUGAACAGGUACAAACUGA | [565-583](19/19) |
| 16 | Cross Sp | 19 | GAAGAAGGCAUAUCAAGAU | AUCUUGAUAUGCCUUCUUC | [2474-2492](19/19) |
| 17 | Cross Sp | 19 | GCCAUCACCUGCAUUCAGU | ACUGAAUGCAGGUGAUGGC | [2433-2451](19/19) |
| 18 | Cross Sp | 19 | CUUGCUGAAGCCAAGAAGA | UCUUCUUGGCUUCAGCAAG | [2103-2121](19/19) |
| 19 | Cross Sp | 19 | GAAUGCCAUGGAUGAGAUU | AAUCUCAUCCAUGGCAUUC | [461-479](19/19) |
| 20 | Cross Sp | 19 | GGCAGUCCAACAAAGACAC | GUGUCUUUGUUGGACUGCC | [1933-1951](19/19) |
| 21 | Cross Sp | 19 | CAAGGUGGAAUCUGGCAGU | ACUGCCAGAUUCCACCUUG | [1920-1938](19/19) |
| 22 | Cross Sp | 19 | UGAAAUUCAAGGUGGAAUC | GAUUCCACCUUGAAUUUCA | [1913-1931](19/19) |
| 23 | Cross Sp | 19 | AUGGUUGGAUGAAAUUCAA | UUGAAUUUCAUCCAACCAU | [1904-1922](19/19) |
| 24 | Cross Sp | 19 | UGUUAUGGUUGGAUGAAAU | AUUUCAUCCAACCAUAACA | [1900-1918](19/19) |
| 25 | Cross Sp | 19 | GUGGAUGGAAGCAUGCCUA | UAGGCAUGCUUCCAUCCAC | [257-275](19/19) |
| 26 | Cross Sp | 19 | GCUGUGUUAUGGUUGGAUG | CAUCCAACCAUAACACAGC | [1896-1914](19/19) |
| 27 | Cross Sp | 19 | AGUCAGCUGUGUUAUGGUU | AACCAUAACACAGCUGACU | [1891-1909](19/19) |
| 28 | Cross Sp | 19 | CUUCUCAACAAAAAGUUCU | AGAACUUUUUGUUGAGAAG | [5046-5064](19/19) |
| 29 | Cross Sp | 19 | UCCUUCUCAACAAAAAGUU | AACUUUUUGUUGAGAAGGA | [5044-5062](19/19) |
| 30 | Cross Sp | 19 | UCUUCCUUCUCAACAAAAA | UUUUUGUUGAGAAGGAAGA | [5041-5059](19/19) |
| 31 | Cross Sp | 19 | UGAUCUUCCUUCUCAACAA | UUGUUGAGAAGGAAGAUCA | [5038-5056](19/19) |
| 32 | Cross Sp | 19 | UCCUGAUCUUCCUUCUCAA | UUGAGAAGGAAGAUCAGGA | [5035-5053](19/19) |
| 33 | Cross Sp | 19 | CUCCUGAUCUUCCUUCUCA | UGAGAAGGAAGAUCAGGAG | [5034-5052](19/19) |
| 34 | Cross Sp | 19 | CAACCUCCUGAUCUUCCUU | AAGGAAGAUCAGGAGGUUG | [5030-5048](19/19) |
| 35 | Cross Sp | 19 | UCAACCUCCUGAUCUUCCU | AGGAAGAUCAGGAGGUUGA | [5029-5047](19/19) |
| 36 | Cross Sp | 19 | AUGUCAACCUCCUGAUCUU | AAGAUCAGGAGGUUGACAU | [5026-5044](19/19) |
| 37 | Cross Sp | 19 | GCUGCAGCUACAGUAUGAA | UUCAUACUGUAGCUGCAGC | [4967-4985](19/19) |
| 38 | Cross Sp | 19 | UGCCCCAGGUGUAUCCAUU | AAUGGAUACACCUGGGGCA | [1354-1372](19/19) |
| 39 | Cross Sp | 19 | CAACAGAAGAAGUUGGAGA | UCUCCAACUUCUUCUGUUG | [4882-4900](19/19) |
| 40 | Cross Sp | 19 | AGAAGAAAGAGAAGAUCCA | UGGAUCUUCUCUUUCUUCU | [4462-4480](19/19) |
| 41 | Cross Sp | 19 | GUUUAAUUGUGGAUGUCAU | AUGACAUCCACAAUUAAAC | [4270-4288](19/19) |
| 42 | Cross Sp | 19 | UCAACACCCACACUCUCCU | AGGAGAGUGUGGGUGUUGA | [4009-4027](19/19) |
| 43 | Cross Sp | 19 | GCUCUGUGGACAAAAUCCC | GGGAUUUUGUCCACAGAGC | [3553-3571](19/19) |
| 44 | Cross Sp | 19 | GAAAUCAACAACAUGAAGA | UCUUCAUGUUGUUGAUUUC | [642-660](19/19) |
| 45 | Cross Sp | 19 | AGGAAAUUAUGGAUGACAA | UUGUCAUCCAUAAUUUCCU | [3331-3349](19/19) |
| 46 | Cross Sp | 19 | AGACUCUCAUCAAUGCUGA | UCAGCAUUGAUGAGAGUCU | [2668-2686](19/19) |
| 47 | Cross Sp | 19 | CAAGACUCUCAUCAAUGCU | AGCAUUGAUGAGAGUCUUG | [2666-2684](19/19) |
| 48 | Cross Sp | 19 | ACAAGACUCUCAUCAAUGC | GCAUUGAUGAGAGUCUUGU | [2665-2683](19/19) |
| 49 | Cross Sp | 19 | AUGACUACAAGACUCUCAU | AUGAGAGUCUUGUAGUCAU | [2659-2677](19/19) |
| 50 | Cross Sp | 19 | GAAGGCAUAUCAAGAUCGG | CCGAUCUUGAUAUGCCUUC | [2477-2495](19/19) |
| 51 | Cross Sp | 19 | UCACAGUGGAGAGGAUACA | UGUAUCCUCUCCACUGUGA | [2451-2469](19/19) |
| 52 | Cross Sp | 19 | CAGUCACAGUGGAGAGGAU | AUCCUCUCCACUGUGACUG | [2448-2466](19/19) |
| 53 | Cross Sp | 19 | CUCAGUUUGUACCUGUUCA | UGAACAGGUACAAACUGAG | [564-582](19/19) |
| 54 | Cross Sp | 19 | CCUGCAUUCAGUCACAGUG | CACUGUGACUGAAUGCAGG | [2440-2458](19/19) |
| 55 | Cross Sp | 19 | CCAUCACCUGCAUUCAGUC | GACUGAAUGCAGGUGAUGG | [2434-2452](19/19) |
| 56 | Cross Sp | 19 | AGAGGUGGAUGGAAGCAUG | CAUGCUUCCAUCCACCUCU | [253-271](19/19) |
| 57 | Cross Sp | 19 | ACAGCAAGUGGGUGAAGCA | UGCUUCACCCACUUGCUGU | [2146-2164](19/19) |
| 58 | Cross Sp | 19 | UUGCUGAAGCCAAGAAGAA | UUCUUCUUGGCUUCAGCAA | [2104-2122](19/19) |
| 59 | Cross Sp | 19 | UGGCAGUCCAACAAAGACA | UGUCUUUGUUGGACUGCCA | [1932-1950](19/19) |
| 60 | Cross Sp | 19 | UCUGGCAGUCCAACAAAGA | UCUUUGUUGGACUGCCAGA | [1930-1948](19/19) |
| 61 | Cross Sp | 19 | AUCUGGCAGUCCAACAAAG | CUUUGUUGGACUGCCAGAU | [1929-1947](19/19) |
| 62 | Cross Sp | 19 | GGAAUCUGGCAGUCCAACA | UGUUGGACUGCCAGAUUCC | [1926-1944](19/19) |
| 63 | Cross Sp | 19 | GUGGAAUCUGGCAGUCCAA | UUGGACUGCCAGAUUCCAC | [1924-1942](19/19) |
| 64 | Cross Sp | 19 | GAAAUUCAAGGUGGAAUCU | AGAUUCCACCUUGAAUUUC | [1914-1932](19/19) |
| 65 | Cross Sp | 19 | GUUGGAUGAAAUUCAAGGU | ACCUUGAAUUUCAUCCAAC | [1907-1925](19/19) |
| 66 | Cross Sp | 19 | GGUUGGAUGAAAUUCAAGG | CCUUGAAUUUCAUCCAACC | [1906-1924](19/19) |
| 67 | Cross Sp | 19 | UAUGGUUGGAUGAAAUUCA | UGAAUUUCAUCCAACCAUA | [1903-1921](19/19) |
| 68 | Cross Sp | 19 | GUUAUGGUUGGAUGAAAUU | AAUUUCAUCCAACCAUAAC | [1901-1919](19/19) |
| 69 | Cross Sp | 19 | GUGUUAUGGUUGGAUGAAA | UUUCAUCCAACCAUAACAC | [1899-1917](19/19) |
| 70 | Cross Sp | 19 | CUGUGUUAUGGUUGGAUGA | UCAUCCAACCAUAACACAG | [1897-1915](19/19) |
| 71 | Cross Sp | 19 | GAGUCAGCUGUGUUAUGGU | ACCAUAACACAGCUGACUC | [1890-1908](19/19) |
| 72 | Cross Sp | 19 | UGAGUCAGCUGUGUUAUGG | CCAUAACACAGCUGACUCA | [1889-1907](19/19) |
| 73 | Cross Sp | 19 | GAUGAGUCAGCUGUGUUAU | AUAACACAGCUGACUCAUC | [1887-1905](19/19) |
| 74 | Cross Sp | 19 | CAGGAUGAGUCAGCUGUGU | ACACAGCUGACUCAUCCUG | [1884-1902](19/19) |
| 75 | Cross Sp | 19 | AUGAAGCCCUGGAUGAAGG | CCUUCAUCCAGGGCUUCAU | [1738-1756](19/19) |
| 76 | Cross Sp | 19 | CCCAGGUGUAUCCAUUUGC | GCAAAUGGAUACACCUGGG | [1357-1375](19/19) |
| 77 | Cross Sp | 19 | UUCCUUCUCAACAAAAAGU | ACUUUUUGUUGAGAAGGAA | [5043-5061](19/19) |
| 78 | Cross Sp | 19 | CCCCAGGUGUAUCCAUUUG | CAAAUGGAUACACCUGGGG | [1356-1374](19/19) |
| 79 | Cross Sp | 19 | CUGAUCUUCCUUCUCAACA | UGUUGAGAAGGAAGAUCAG | [5037-5055](19/19) |
| 80 | Cross Sp | 19 | CCUGAUCUUCCUUCUCAAC | GUUGAGAAGGAAGAUCAGG | [5036-5054](19/19) |
| 81 | Cross Sp | 19 | ACCUCCUGAUCUUCCUUCU | AGAAGGAAGAUCAGGAGGU | [5032-5050](19/19) |
| 82 | Cross Sp | 19 | GCCCCAGGUGUAUCCAUUU | AAAUGGAUACACCUGGGGC | [1355-1373](19/19) |
| 83 | Cross Sp | 19 | UGCAGCUACAGUAUGAAGG | CCUUCAUACUGUAGCUGCA | [4969-4987](19/19) |
| 84 | Cross Sp | 19 | UGCUGCAGCUACAGUAUGA | UCAUACUGUAGCUGCAGCA | [4966-4984](19/19) |
| 85 | Cross Sp | 19 | CAGAAGAAGUUGGAGACUU | AAGUCUCCAACUUCUUCUG | [4885-4903](19/19) |
| 86 | Cross Sp | 19 | UCCAACAGAAGAAGUUGGA | UCCAACUUCUUCUGUUGGA | [4880-4898](19/19) |
| 87 | Cross Sp | 19 | GUCCAACAGAAGAAGUUGG | CCAACUUCUUCUGUUGGAC | [4879-4897](19/19) |
| 88 | Cross Sp | 19 | AGAAAGAGAAGAUCCAGAC | GUCUGGAUCUUCUCUUUCU | [4465-4483](19/19) |
| 89 | Cross Sp | 19 | CAGGAGAGACCUUGACUGA | UCAGUCAAGGUCUCUCCUG | [4300-4318](19/19) |
| 90 | Cross Sp | 19 | UUGUGGAUGUCAUCCGGUU | AACCGGAUGACAUCCACAA | [4276-4294](19/19) |
| 91 | Cross Sp | 19 | CAAAACGUUUAAUUGUGGA | UCCACAAUUAAACGUUUUG | [4264-4282](19/19) |
| 92 | Cross Sp | 19 | ACAAAACGUUUAAUUGUGG | CCACAAUUAAACGUUUUGU | [4263-4281](19/19) |
| 93 | Cross Sp | 19 | CUUACUGAAUACAAAACGU | ACGUUUUGUAUUCAGUAAG | [4253-4271](19/19) |
| 94 | Cross Sp | 19 | AACACCCACACUCUCCUGU | ACAGGAGAGUGUGGGUGUU | [4011-4029](19/19) |
| 95 | Cross Sp | 19 | CAUCAACACCCACACUCUC | GAGAGUGUGGGUGUUGAUG | [4007-4025](19/19) |
| 96 | Cross Sp | 19 | UCAUCAACACCCACACUCU | AGAGUGUGGGUGUUGAUGA | [4006-4024](19/19) |
| 97 | Cross Sp | 19 | AUCAUCAACACCCACACUC | GAGUGUGGGUGUUGAUGAU | [4005-4023](19/19) |
| 98 | Cross Sp | 19 | GAAAUUCAGACGGUUUUUC | GAAAAACCGUCUGAAUUUC | [3881-3899](19/19) |
| 99 | Cross Sp | 19 | CCAGAAAUUCAGACGGUUU | AAACCGUCUGAAUUUCUGG | [3878-3896](19/19) |
| 100 | Cross Sp | 19 | CUACCAGAAAUUCAGACGG | CCGUCUGAAUUUCUGGUAG | [3875-3893](19/19) |
| 101 | Cross Sp | 19 | GUCCUACCAGAAAUUCAGA | UCUGAAUUUCUGGUAGGAC | [3872-3890](19/19) |
| 102 | Cross Sp | 19 | AGUCCUACCAGAAAUUCAG | CUGAAUUUCUGGUAGGACU | [3871-3889](19/19) |
| 103 | Cross Sp | 19 | CAGUCCUACCAGAAAUUCA | UGAAUUUCUGGUAGGACUG | [3870-3888](19/19) |
| 104 | Cross Sp | 19 | CAGCUCUGUGGACAAAAUC | GAUUUUGUCCACAGAGCUG | [3551-3569](19/19) |
| 105 | Cross Sp | 19 | UGCAGGAUGUGGUUUCCCA | UGGGAAACCACAUCCUGCA | [2881-2899](19/19) |
| 106 | Cross Sp | 19 | GAUGACUACAAGACUCUCA | UGAGAGUCUUGUAGUCAUC | [2658-2676](19/19) |
| 107 | Cross Sp | 19 | UUAUCAAAAUCCAGGCUUU | AAAGCCUGGAUUUUGAUAA | [2617-2635](19/19) |
| 108 | Cross Sp | 19 | UCACCUGCAUUCAGUCACA | UGUGACUGAAUGCAGGUGA | [2437-2455](19/19) |
| 109 | Cross Sp | 19 | AGAAAUUCAGACGGUUUUU | AAAAACCGUCUGAAUUUCU | [3880-3898](19/19) |
| 110 | Cross Sp | 19 | ACCAGAAAUUCAGACGGUU | AACCGUCUGAAUUUCUGGU | [3877-3895](19/19) |
| 111 | Cross Sp | 19 | UACCAGAAAUUCAGACGGU | ACCGUCUGAAUUUCUGGUA | [3876-3894](19/19) |
| 112 | Cross Sp | 19 | CCUACCAGAAAUUCAGACG | CGUCUGAAUUUCUGGUAGG | [3874-3892](19/19) |
| 113 | Cross Sp | 19 | UCCUACCAGAAAUUCAGAC | GUCUGAAUUUCUGGUAGGA | [3873-3891](19/19) |
| 114 | Cross Sp | 19 | CUCUGUGGACAAAAUCCCU | AGGGAUUUUGUCCACAGAG | [3554-3572](19/19) |
| 115 | Cross Sp | 19 | ACUACAAGACUCUCAUCAA | UUGAUGAGAGUCUUGUAGU | [2662-2680](19/19) |
| 116 | Cross Sp | 19 | UGACUACAAGACUCUCAUC | GAUGAGAGUCUUGUAGUCA | [2660-2678](19/19) |
| 117 | Cross Sp | 19 | CAUUAUCAAAAUCCAGGCU | AGCCUGGAUUUUGAUAAUG | [2615-2633](19/19) |
| 118 | Cross Sp | 19 | ACAUUAUCAAAAUCCAGGC | GCCUGGAUUUUGAUAAUGU | [2614-2632](19/19) |
| 119 | Cross Sp | 19 | GACAUUAUCAAAAUCCAGG | CCUGGAUUUUGAUAAUGUC | [2613-2631](19/19) |
| 120 | Cross Sp | 19 | UGACAUUAUCAAAAUCCAG | CUGGAUUUUGAUAAUGUCA | [2612-2630](19/19) |
| 121 | Cross Sp | 19 | AUCACCUGCAUUCAGUCAC | GUGACUGAAUGCAGGUGAU | [2436-2454](19/19) |
| 122 | Cross Sp | 19 | CAUCACCUGCAUUCAGUCA | UGACUGAAUGCAGGUGAUG | [2435-2453](19/19) |
| 123 | Cross Sp | 19 | GUUCGACAGGAAUUCCGAU | AUCGGAAUUCCUGUCGAAC | [2382-2400](19/19) |
| 124 | Cross Sp | 19 | AUGCCAUGGAUGAGAUUGG | CCAAUCUCAUCCAUGGCAU | [463-481](19/19) |
| 125 | Cross Sp | 19 | AACAGCAAGUGGGUGAAGC | GCUUCACCCACUUGCUGUU | [2145-2163](19/19) |
| 126 | Cross Sp | 19 | AUAACAGCAAGUGGGUGAA | UUCACCCACUUGCUGUUAU | [2143-2161](19/19) |
| 127 | Cross Sp | 19 | AAUCUGGCAGUCCAACAAA | UUUGUUGGACUGCCAGAUU | [1928-1946](19/19) |
| 128 | Cross Sp | 19 | UCAAGGUGGAAUCUGGCAG | CUGCCAGAUUCCACCUUGA | [1919-1937](19/19) |
| 129 | Cross Sp | 19 | AAUUCAAGGUGGAAUCUGG | CCAGAUUCCACCUUGAAUU | [1916-1934](19/19) |
| 130 | Cross Sp | 19 | AUGAAAUUCAAGGUGGAAU | AUUCCACCUUGAAUUUCAU | [1912-1930](19/19) |
| 131 | Cross Sp | 19 | UUGGAUGAAAUUCAAGGUG | CACCUUGAAUUUCAUCCAA | [1908-1926](19/19) |
| 132 | Cross Sp | 19 | UAAUGAAGCCCUGGAUGAA | UUCAUCCAGGGCUUCAUUA | [1736-1754](19/19) |
| 133 | Cross Sp | 19 | CUGCAGCUACAGUAUGAAG | CUUCAUACUGUAGCUGCAG | [4968-4986](19/19) |
| 134 | Cross Sp | 19 | UUAAUGAAGCCCUGGAUGA | UCAUCCAGGGCUUCAUUAA | [1735-1753](19/19) |
| 135 | Cross Sp | 19 | AACAGAAGAAGUUGGAGAC | GUCUCCAACUUCUUCUGUU | [4883-4901](19/19) |
| 136 | Cross Sp | 19 | CCAACAGAAGAAGUUGGAG | CUCCAACUUCUUCUGUUGG | [4881-4899](19/19) |
| 137 | Cross Sp | 19 | UGGAUGUCAUCCGGUUCCA | UGGAACCGGAUGACAUCCA | [4279-4297](19/19) |
| 138 | Cross Sp | 19 | CGUUUAAUUGUGGAUGUCA | UGACAUCCACAAUUAAACG | [4269-4287](19/19) |
| 139 | Cross Sp | 19 | ACGUUUAAUUGUGGAUGUC | GACAUCCACAAUUAAACGU | [4268-4286](19/19) |
| 140 | Cross Sp | 19 | AACGUUUAAUUGUGGAUGU | ACAUCCACAAUUAAACGUU | [4267-4285](19/19) |
| 141 | Cross Sp | 19 | AAACGUUUAAUUGUGGAUG | CAUCCACAAUUAAACGUUU | [4266-4284](19/19) |
| 142 | Cross Sp | 19 | ACACCCACACUCUCCUGUU | AACAGGAGAGUGUGGGUGU | [4012-4030](19/19) |
| 143 | Cross Sp | 19 | AUUCAGACGGUUUUUCCAA | UUGGAAAAACCGUCUGAAU | [3884-3902](19/19) |
| 144 | Cross Sp | 19 | AAUUCAGACGGUUUUUCCA | UGGAAAAACCGUCUGAAUU | [3883-3901](19/19) |
| 145 | Cross Sp | 19 | AAAUUCAGACGGUUUUUCC | GGAAAAACCGUCUGAAUUU | [3882-3900](19/19) |
| 146 | Cross Sp | 19 | AAGAGGUGGAUGGAAGCAU | AUGCUUCCAUCCACCUCUU | [252-270](19/19) |
| 147 | Cross Sp | 19 | AGCUCUGUGGACAAAAUCC | GGAUUUUGUCCACAGAGCU | [3552-3570](19/19) |
| 148 | Cross Sp | 19 | UACAAGACUCUCAUCAAUG | CAUUGAUGAGAGUCUUGUA | [2664-2682](19/19) |
| 149 | Cross Sp | 19 | AUUAUCAAAAUCCAGGCUU | AAGCCUGGAUUUUGAUAAU | [2616-2634](19/19) |
| 150 | Cross Sp | 19 | UCGACAGGAAUUCCGAUCC | GGAUCGGAAUUCCUGUCGA | [2384-2402](19/19) |
| 151 | Cross Sp | 19 | UUCGACAGGAAUUCCGAUC | GAUCGGAAUUCCUGUCGAA | [2383-2401](19/19) |
| 152 | Cross Sp | 19 | AGCAAGUGGGUGAAGCACU | AGUGCUUCACCCACUUGCU | [2148-2166](19/19) |
| 153 | Cross Sp | 19 | UAACAGCAAGUGGGUGAAG | CUUCACCCACUUGCUGUUA | [2144-2162](19/19) |
| 154 | Cross Sp | 19 | AAUAACAGCAAGUGGGUGA | UCACCCACUUGCUGUUAUU | [2142-2160](19/19) |
| 155 | Cross Sp | 19 | UGGAAUCUGGCAGUCCAAC | GUUGGACUGCCAGAUUCCA | [1925-1943](19/19) |
| 156 | Cross Sp | 19 | AAGGUGGAAUCUGGCAGUC | GACUGCCAGAUUCCACCUU | [1921-1939](19/19) |
| 157 | Human | 19 | CCACUUACCAGGAUAUACU | AGUAUAUCCUGGUAAGUGG | [868-886](19/19) |
| 158 | Human | 19 | CGAAUGCCAUGCUUGUAAA | UUUACAAGCAUGGCAUUCG | [829-847](19/19) |
| 159 | Human | 19 | GCACAUAGUUGCCUUUGUA | UACAAAGGCAACUAUGUGC | [7157-7175](19/19) |
| 160 | Human | 19 | CCACACAGUUCUCAUUACU | AGUAAUGAGAACUGUGUGG | [7007-7025](19/19) |
| 161 | Human | 19 | CAAGAAUGUUGAUGAUGAU | AUCAUCAUCAACAUUCUUG | [6928-6946](19/19) |
| 162 | Human | 19 | CAACACAAUUGCUAAUGCA | UGCAUUAGCAAUUGUGUUG | [6889-6907](19/19) |
| 163 | Human | 19 | GCAAUAAUGAGCAGAGUUU | AAACUCUGCUCAUUAUUGC | [6820-6838](19/19) |
| 164 | Human | 19 | CCACUACCCAGACCUUGUU | AACAAGGUCUGGGUAGUGG | [6636-6654](19/19) |
| 165 | Human | 19 | GGAGUGCCAUGGAAGGAUU | AAUCCUUCCAUGGCACUCC | [6615-6633](19/19) |
| 166 | Human | 19 | CAAGUCUAAACAUUGCAUU | AAUGCAAUGUUUAGACUUG | [6299-6317](19/19) |
| 167 | Human | 19 | CCAAUUUUUUCCCCAUUUU | AAAAUGGGGAAAAAAUUGG | [6199-6217](19/19) |
| 168 | Human | 19 | GCACCUGCCUGUUUUUGUU | AACAAAAACAGGCAGGUGC | [5815-5833](19/19) |
| 169 | Human | 19 | CAAGGCACCUGCCUGUUUU | AAAACAGGCAGGUGCCUUG | [5811-5829](19/19) |
| 170 | Human | 19 | GGAAGCAUUUCUAGAAAAA | UUUUUCUAGAAAUGCUUCC | [5669-5687](19/19) |
| 171 | Human | 19 | GGAAACUCCACUAAAAUUU | AAAUUUUAGUGGAGUUUCC | [5332-5350](19/19) |
| 172 | Human | 19 | CCAUUACUUAGGAAAGAGU | ACUCUUUCCUAAGUAAUGG | [5313-5331](19/19) |
| 173 | Human | 19 | GGAAAGAAAAGCAAAAAGA | UCUUUUUGCUUUUCUUUCC | [4758-4776](19/19) |
| 174 | Human | 19 | GGAAAUGAAAGGAAAGAAA | UUUCUUUCCUUUCAUUUCC | [4748-4766](19/19) |
| 175 | Human | 19 | CUAGGGAAAUGAAAGGAAA | UUUCCUUUCAUUUCCCUAG | [4744-4762](19/19) |
| 176 | Human | 19 | CAAGGGCAAAGUCUCCAAA | UUUGGAGACUUUGCCCUUG | [4721-4739](19/19) |
| 177 | Human | 19 | GAACCGUGGACCCAAAGAA | UUCUUUGGGUCCACGGUUC | [4510-4528](19/19) |
| 178 | Human | 19 | CAAGAUGUAUCUACUGUAU | AUACAGUAGAUACAUCUUG | [538-556](19/19) |
| 179 | Human | 19 | GGAAAGCUCUGGCAAUUUA | UAAAUUGCCAGAGCUUUCC | [4127-4145](19/19) |
| 180 | Human | 19 | CCAUGGAUGAGAUUGGAUU | AAUCCAAUCUCAUCCAUGG | [466-484](19/19) |
| 181 | Human | 19 | CAAGCAAACUGCCCUAUGA | UCAUAGGGCAGUUUGCUUG | [3427-3445](19/19) |
| 182 | Human | 19 | CCACUUUAGACACACUGAU | AUCAGUGUGUCUAAAGUGG | [425-443](19/19) |
| 183 | Human | 19 | GCAAACCAAUCCCACCUAU | AUAGGUGGGAUUGGUUUGC | [3038-3056](19/19) |
| 184 | Human | 19 | CGAGAACAGACCAGAUACA | UGUAUCUGGUCUGUUCUCG | [393-411](19/19) |
| 185 | Human | 19 | CCACAAAGAUGAAGUUGUA | UACAACUUCAUCUUUGUGG | [2513-2531](19/19) |
| 186 | Human | 19 | GGAUGAAUUUCCUGAAGAA | UUCUUCAGGAAAUUCAUCC | [2404-2422](19/19) |
| 187 | Human | 19 | GGAUGAACCUCCAAAUUUU | AAAAUUUGGAGGUUCAUCC | [2222-2240](19/19) |
| 188 | Human | 19 | CCAUUAAUGAGGCAGUAGA | UCUACUGCCUCAUUAAUGG | [1987-2005](19/19) |
| 189 | Human | 19 | CCAAUAUUGAGGAAGAAAA | UUUUCUUCCUCAAUAUUGG | [1561-1579](19/19) |
| 190 | Human | 19 | GGAAUCAGGAGAUGUGAAU | AUUCACAUCUCCUGAUUCC | [1502-1520](19/19) |
| 191 | Human | 19 | GUACCUUUGUCAUUUGGAA | UUCCAAAUGACAAAGGUAC | [227-245](19/19) |
| 192 | Human | 19 | GAGAUGCACUGGCCUUGUU | AACAAGGCCAGUGCAUCUC | [1057-1075](19/19) |
| 193 | Human | 19 | CGACCUGGCUUUAGAACAA | UUGUUCUAAAGCCAGGUCG | [1037-1055](19/19) |
| 194 | Human | 19 | CACGCAAGCUGAAAUUCAA | UUGAAUUUCAGCUUGCGUG | [974-992](19/19) |
| 195 | Human | 19 | GCUCACGCAAGCUGAAAUU | AAUUUCAGCUUGCGUGAGC | [971-989](19/19) |
| 196 | Human | 19 | GGAGAUGGAUGAAAGGAGA | UCUCCUUUCAUCCAUCUCC | [188-206](19/19) |
| 197 | Human | 19 | GAGAGAAAGAGAUGUUUAU | AUAAACAUCUCUUUCUCUC | [944-962](19/19) |
| 198 | Human | 19 | CUCAGAGAGAGAAAGAGAU | AUCUCUUUCUCUCUCUGAG | [938-956](19/19) |
| 199 | Human | 19 | GGACAGAAAACUCAGAGAG | CUCUCUGAGUUUUCUGUCC | [928-946](19/19) |
| 200 | Human | 19 | AAACAGGACAGAAAACUCA | UGAGUUUUCUGUCCUGUUU | [923-941](19/19) |
| 201 | Human | 19 | GCUAAAAACAGGACAGAAA | UUUCUGUCCUGUUUUUAGC | [918-936](19/19) |
| 202 | Human | 19 | GACAAAAUGACAAAUGCUA | UAGCAUUUGUCAUUUUGUC | [903-921](19/19) |
| 203 | Human | 19 | CCAGGAUAUACUUUACCAG | CUGGUAAAGUAUAUCCUGG | [875-893](19/19) |
| 204 | Human | 19 | CAUCCACUUACCAGGAUAU | AUAUCCUGGUAAGUGGAUG | [865-883](19/19) |
| 205 | Human | 19 | GCAUCCACUUACCAGGAUA | UAUCCUGGUAAGUGGAUGC | [864-882](19/19) |
| 206 | Human | 19 | CCAUGCUUGUAAAUCUUGA | UCAAGAUUUACAAGCAUGG | [835-853](19/19) |
| 207 | Human | 19 | CACAUUUGCAGCUUUGAAA | UUUCAAAGCUGCAAAUGUG | [806-824](19/19) |
| 208 | Human | 19 | CGACACAUUUGCAGCUUUG | CAAAGCUGCAAAUGUGUCG | [803-821](19/19) |
| 209 | Human | 19 | GCCAUCAUGACAUUUACCA | UGGUAAAUGUCAUGAUGGC | [7090-7108](19/19) |
| 210 | Human | 19 | CCAGCCAUCAUGACAUUUA | UAAAUGUCAUGAUGGCUGG | [7087-7105](19/19) |
| 211 | Human | 19 | GACCAGCCAUCAUGACAUU | AAUGUCAUGAUGGCUGGUC | [7085-7103](19/19) |
| 212 | Human | 19 | CGACCAGCCAUCAUGACAU | AUGUCAUGAUGGCUGGUCG | [7084-7102](19/19) |
| 213 | Human | 19 | ACACAGUUCUCAUUACUGU | ACAGUAAUGAGAACUGUGU | [7009-7027](19/19) |
| 214 | Human | 19 | AAACCACACAGUUCUCAUU | AAUGAGAACUGUGUGGUUU | [7004-7022](19/19) |
| 215 | Human | 19 | GUUGAAACCACACAGUUCU | AGAACUGUGUGGUUUCAAC | [7000-7018](19/19) |
| 216 | Human | 19 | ACAAACUCAUGGCGUUGAA | UUCAACGCCAUGAGUUUGU | [6987-7005](19/19) |
| 217 | Human | 19 | CCAACAAACUCAUGGCGUU | AACGCCAUGAGUUUGUUGG | [6984-7002](19/19) |
| 218 | Human | 19 | GAUGCCCCAACAAACUCAU | AUGAGUUUGUUGGGGCAUC | [6978-6996](19/19) |
| 219 | Human | 19 | GGAUGCCCCAACAAACUCA | UGAGUUUGUUGGGGCAUCC | [6977-6995](19/19) |
| 220 | Human | 19 | AUAGAAUUGUGGCUUUAGU | ACUAAAGCCACAAUUCUAU | [6950-6968](19/19) |
| 221 | Human | 19 | GAAUGUUGAUGAUGAUGAU | AUCAUCAUCAUCAACAUUC | [6931-6949](19/19) |
| 222 | Human | 19 | CUUCAAGAAUGUUGAUGAU | AUCAUCAACAUUCUUGAAG | [6925-6943](19/19) |
| 223 | Human | 19 | GCAGAAUAGUGUAAAAUGC | GCAUUUUACACUAUUCUGC | [6905-6923](19/19) |
| 224 | Human | 19 | AUAGUUCCCUCAACACAAU | AUUGUGUUGAGGGAACUAU | [6879-6897](19/19) |
| 225 | Human | 19 | AGAGUUUACAGCUCCUUUA | UAAAGGAGCUGUAAACUCU | [6832-6850](19/19) |
| 226 | Human | 19 | CCAAGACAGGCAAUAAUGA | UCAUUAUUGCCUGUCUUGG | [6811-6829](19/19) |
| 227 | Human | 19 | CCACAUCCAAGACAGGCAA | UUGCCUGUCUUGGAUGUGG | [6805-6823](19/19) |
| 228 | Human | 19 | CCAAAGGCCACAUCCAAGA | UCUUGGAUGUGGCCUUUGG | [6798-6816](19/19) |
| 229 | Human | 19 | CCUUAAAAGAGGCUAUGAA | UUCAUAGCCUCUUUUAAGG | [6777-6795](19/19) |
| 230 | Human | 19 | CAACCCUGUUUCCUUAAAA | UUUUAAGGAAACAGGGUUG | [6766-6784](19/19) |
| 231 | Human | 19 | CUGGGACCAACCAAAGUGU | ACACUUUGGUUGGUCCCAG | [6745-6763](19/19) |
| 232 | Human | 19 | CAACUAGCAUUGCCUCAAA | UUUGAGGCAAUGCUAGUUG | [6724-6742](19/19) |
| 233 | Human | 19 | GAAACAUUUUCCUCAGAUU | AAUCUGAGGAAAAUGUUUC | [6688-6706](19/19) |
| 234 | Human | 19 | CCCAGACCUUGUUUUUUGU | ACAAAAAACAAGGUCUGGG | [6642-6660](19/19) |
| 235 | Human | 19 | CUACCCAGACCUUGUUUUU | AAAAACAAGGUCUGGGUAG | [6639-6657](19/19) |
| 236 | Human | 19 | ACAUGCUGCUGUUAUUGCU | AGCAAUAACAGCAGCAUGU | [752-770](19/19) |
| 237 | Human | 19 | UUACUCAGGUGCAUGGUUA | UAACCAUGCACCUGAGUAA | [6578-6596](19/19) |
| 238 | Human | 19 | GCAUUGGAGAAGUAUUUUU | AAAAAUACUUCUCCAAUGC | [6549-6567](19/19) |
| 239 | Human | 19 | GUAGUGUGUCCCUAUUAUA | UAUAAUAGGGACACACUAC | [6527-6545](19/19) |
| 240 | Human | 19 | CCAAUAAACUGCUUUAAUG | CAUUAAAGCAGUUUAUUGG | [6495-6513](19/19) |
| 241 | Human | 19 | GGAUGAAGCCGCAUUACAU | AUGUAAUGCGGCUUCAUCC | [737-755](19/19) |
| 242 | Human | 19 | GGAUAAUGAAAGACUUACU | AGUAAGUCUUUCAUUAUCC | [164-182](19/19) |
| 243 | Human | 19 | GCAUUUAGAAAGCUUUUGU | ACAAAAGCUUUCUAAAUGC | [6313-6331](19/19) |
| 244 | Human | 19 | CCAGUUUUCUGGGCUUAGU | ACUAAGCCCAGAAAACUGG | [6267-6285](19/19) |
| 245 | Human | 19 | CCACAUUGUGCCUUUAUUU | AAAUAAAGGCACAAUGUGG | [6240-6258](19/19) |
| 246 | Human | 19 | CCCACAUUGUGCCUUUAUU | AAUAAAGGCACAAUGUGGG | [6239-6257](19/19) |
| 247 | Human | 19 | CCCAUUUUUUCUCCUUUUU | AAAAAGGAGAAAAAAUGGG | [6210-6228](19/19) |
| 248 | Human | 19 | GGGGCAUCUUGGCUAAUGA | UCAUUAGCCAAGAUGCCCC | [709-727](19/19) |
| 249 | Human | 19 | CUAUGUUACACGUGAGAUU | AAUCUCACGUGUAACAUAG | [6176-6194](19/19) |
| 250 | Human | 19 | GGAGAUAAUUCUGCCUUUA | UAAAGGCAGAAUUAUCUCC | [6126-6144](19/19) |
| 251 | Human | 19 | GCAGUGUUCAGGGAGAUAA | UUAUCUCCCUGAACACUGC | [6115-6133](19/19) |
| 252 | Human | 19 | GGAUUUAUCUGCAGUGUUC | GAACACUGCAGAUAAAUCC | [6105-6123](19/19) |
| 253 | Human | 19 | AUAGCUAAAACACUAGGAU | AUCCUAGUGUUUUAGCUAU | [6090-6108](19/19) |

FOR 21
STRETCHES:

| | | | | | |
|---|---|---|---|---|---|
| 1 | Cross Sp | 21 | GGAAUCUGGCAGUCCAACAAA | UUUGUUGGACUGCCAGAUUCC | [1926-1946](21/21) |
| 2 | Cross Sp | 21 | CCAGAAAUUCAGACGGUUUUU | AAAAACCGUCUGAAUUUCUGG | [3878-3898](21/21) |
| 3 | Cross Sp | 21 | GAAGAAAUCAACAACAUGAAG | CUUCAUGUUGUUGAUUUCUUC | [639-659](21/21) |
| 4 | Cross Sp | 21 | GUGUUAUGGUUGGAUGAAAUU | AAUUUCAUCCAACCAUAACAC | [1899-1919](21/21) |
| 5 | Cross Sp | 21 | UGAUCUUCCUUCUCAACAAAA | UUUUGUUGAGAAGGAAGAUCA | [5038-5058](21/21) |
| 6 | Cross Sp | 21 | GUCAACCUCCUGAUCUUCCUU | AAGGAAGAUCAGGAGGUUGAC | [5028-5048](21/21) |
| 7 | Cross Sp | 21 | CAACAGAAGAAGUUGGAGACU | AGUCUCCAACUUCUUCUGUUG | [4882-4902](21/21) |
| 8 | Cross Sp | 21 | CCAGGAGAGACCUUGACUGAA | UUCAGUCAAGGUCUCUCCUGG | [4299-4319](21/21) |
| 9 | Cross Sp | 21 | CUGCCCCAGGUGUAUCCAUUU | AAAUGGAUACACCUGGGGCAG | [1353-1373](21/21) |
| 10 | Cross Sp | 21 | CAACACCCACACUCUCCUGUU | AACAGGAGAGUGUGGGUGUUG | [4010-4030](21/21) |
| 11 | Cross Sp | 21 | CAGUCCUACCAGAAAUUCAGA | UCUGAAUUUCUGGUAGGACUG | [3870-3890](21/21) |
| 12 | Cross Sp | 21 | UGACUACAAGACUCUCAUCAA | UUGAUGAGAGUCUUGUAGUCA | [2660-2680](21/21) |
| 13 | Cross Sp | 21 | UCAGUCACAGUGGAGAGGAUA | UAUCCUCUCCACUGUGACUGA | [2447-2467](21/21) |
| 14 | Cross Sp | 21 | AAUCUGGCAGUCCAACAAAGA | UCUUUGUUGGACUGCCAGAUU | [1928-1948](21/21) |
| 15 | Cross Sp | 21 | AUGAAAUUCAAGGUGGAAUCU | AGAUUCCACCUUGAAUUUCAU | [1912-1932](21/21) |
| 16 | Cross Sp | 21 | GCUGUGUUAUGGUUGGAUGAA | UUCAUCCAACCAUAACACAGC | [1896-1916](21/21) |
| 17 | Cross Sp | 21 | GAGUCAGCUGUGUUAUGGUUG | CAACCAUAACACAGCUGACUC | [1890-1910](21/21) |
| 18 | Cross Sp | 21 | AGGAUGAGUCAGCUGUGUUAU | AUAACACAGCUGACUCAUCCU | [1885-1905](21/21) |
| 19 | Cross Sp | 21 | CCUUCUCAACAAAAAGUUCUA | UAGAACUUUUUGUUGAGAAGG | [5045-5065](21/21) |
| 20 | Cross Sp | 21 | CUUCCUUCUCAACAAAAAGUU | AACUUUUUGUUGAGAAGGAAG | [5042-5062](21/21) |
| 21 | Cross Sp | 21 | CUGAUCUUCCUUCUCAACAAA | UUUGUUGAGAAGGAAGAUCAG | [5037-5057](21/21) |
| 22 | Cross Sp | 21 | CUCCUGAUCUUCCUUCUCAAC | GUUGAGAAGGAAGAUCAGGAG | [5034-5054](21/21) |
| 23 | Cross Sp | 21 | CCUCCUGAUCUUCCUUCUCAA | UUGAGAAGGAAGAUCAGGAGG | [5033-5053](21/21) |
| 24 | Cross Sp | 21 | CAACCUCCUGAUCUUCCUUCU | AGAAGGAAGAUCAGGAGGUUG | [5030-5050](21/21) |
| 25 | Cross Sp | 21 | GUCCAACAGAAGAAGUUGGAG | CUCCAACUUCUUCUGUUCGGAC | [4879-4899](21/21) |
| 26 | Cross Sp | 21 | GAGAAGAAAGAGAAGAUCCAG | CUGGAUCUUCUCUUUCUUCUC | [4461-4481](21/21) |
| 27 | Cross Sp | 21 | CGUUUAAUUGUGGAUGUCAUC | GAUGACAUCCACAAUUAAACG | [4269-4289](21/21) |
| 28 | Cross Sp | 21 | GAAAUUCAGACGGUUUUUCCA | UGGAAAAACCGUCUGAAUUUC | [3881-3901](21/21) |
| 29 | Cross Sp | 21 | CUACCAGAAAUUCAGACGGUU | AACCGUCUGAAUUUCUGGUAG | [3875-3895](21/21) |
| 30 | Cross Sp | 21 | GUCAGCUCUGUGGACAAAAUC | GAUUUUGUCCACAGAGCUGAC | [3549-3569](21/21) |
| 31 | Cross Sp | 21 | GAAGGAAAUUAUGGAUGACAA | UUGUCAUCCAUAAUUUCCUUC | [3329-3349](21/21) |
| 32 | Cross Sp | 21 | CAAGACUCUCAUCAAUGCUGA | UCAGCAUUGAUGAGAGUCUUG | [2666-2686](21/21) |
| 33 | Cross Sp | 21 | UACAAGACUCUCAUCAAUGCU | AGCAUUGAUGAGAGUCUUGUA | [2664-2684](21/21) |
| 34 | Cross Sp | 21 | GAUGACUACAAGACUCUCAUC | GAUGAGAGUCUUGUAGUCAUC | [2658-2678](21/21) |
| 35 | Cross Sp | 21 | GAAGAAGGCAUAUCAAGAUCG | CGAUCUUGAUAUGCCUUCUUC | [2474-2494](21/21) |
| 36 | Cross Sp | 21 | GUCACAGUGGAGAGGAUACAA | UUGUAUCCUCUCCACUGUGAC | [2450-2470](21/21) |
| 37 | Cross Sp | 21 | GCAUUCAGUCACAGUGGAGAG | CUCUCCACUGUGACUGAAUGC | [2443-2463](21/21) |
| 38 | Cross Sp | 21 | CUGCAUUCAGUCACAGUGGAG | CUCCACUGUGACUGAAUGCAG | [2441-2461](21/21) |
| 39 | Cross Sp | 21 | UCACCUGCAUUCAGUCACAGU | ACUGUGACUGAAUGCAGGUGA | [2437-2457](21/21) |
| 40 | Cross Sp | 21 | CAUCACCUGCAUUCAGUCACA | UGUGACUGAAUGCAGGUGAUG | [2435-2455](21/21) |
| 41 | Cross Sp | 21 | CCAUCACCUGCAUUCAGUCAC | GUGACUGAAUGCAGGUGAUGG | [2434-2454](21/21) |
| 42 | Cross Sp | 21 | GCCAUCACCUGCAUUCAGUCA | UGACUGAAUGCAGGUGAUGGC | [2433-2453](21/21) |
| 43 | Cross Sp | 21 | UAACAGCAAGUGGGUGAAGCA | UGCUUCACCCACUUGCUGUUA | [2144-2164](21/21) |
| 44 | Cross Sp | 21 | GCAGUCCAACAAAGACACCCA | UGGGUGUCUUUGUUUGACUGC | [1934-1954](21/21) |
| 45 | Cross Sp | 21 | CUGGCAGUCCAACAAAGACAC | GUGUCUUUGUUGGACUGCCAG | [1931-1951](21/21) |
| 46 | Cross Sp | 21 | AGGUGGAAUCUGGCAGUCCAA | UUGGACUGCCAGAUUCCACCU | [1922-1942](21/21) |
| 47 | Cross Sp | 21 | UGAAUGCCAUGGAUGAGAUUG | CAAUCUCAUCCAUGGCAUUCA | [460-480](21/21) |
| 48 | Cross Sp | 21 | GGAUGAAAUUCAAGGUGGAAU | AUUCCACCUUGAAUUUCAUCC | [1910-1930](21/21) |
| 49 | Cross Sp | 21 | UGGAUGAAAUUCAAGGUGGAA | UUCCACCUUGAAUUUCAUCCA | [1909-1929](21/21) |
| 50 | Cross Sp | 21 | GGUUUGAUGAAAUUCAAGGUG | CACCUUGAAUUUCAUCCAACC | [1906-1926](21/21) |
| 51 | Cross Sp | 21 | UAUGGUUGGAUGAAAUUCAAG | CUUGAAUUUCAUCCAACCAUA | [1903-1923](21/21) |
| 52 | Cross Sp | 21 | AGCUGUGUUAUGGUUGGAUGA | UCAUCCAACCAUAACACAGCU | [1895-1915](21/21) |
| 53 | Cross Sp | 21 | UCAGCUGUGUUAUGGUUGGAU | AUCCAACCAUAACACAGCUGA | [1893-1913](21/21) |
| 54 | Cross Sp | 21 | GUCAGCUGUGUUAUGGUUGGA | UCCAACCAUAACACAGCUGAC | [1892-1912](21/21) |
| 55 | Cross Sp | 21 | UGAGUCAGCUGUGUUAUGGUU | AACCAUAACACAGCUGACUCA | [1889-1909](21/21) |
| 56 | Cross Sp | 21 | AUGAGUCAGCUGUGUUAUGGU | ACCAUAACACAGCUGACUCAU | [1888-1908](21/21) |
| 57 | Cross Sp | 21 | GGAUGAGUCAGCUGUGUUAUG | CAUAACACAGCUGACUCAUCC | [1866-1906](21/21) |
| 58 | Cross Sp | 21 | CAGGAUGAGUCAGCUGUGUUA | UAACACAGCUGACUCAUCCUG | [1884-1904](21/21) |
| 59 | Cross Sp | 21 | UUCCUUCUCAACAAAAAGUUC | GAACUUUUUGUUGAGAAGGAA | [5043-5063](21/21) |
| 60 | Cross Sp | 21 | AUCUUCCUUCUCAACAAAAAG | CUUUUUGUUGAGAAGGAAGAU | [5040-5060](21/21) |
| 61 | Cross Sp | 21 | GAUCUUCCUUCUCAACAAAAA | UUUUUGUUGAGAAGGAAGAUC | [5039-5059](21/21) |
| 62 | Cross Sp | 21 | CCUGAUCUUCCUUCUCAACAA | UUGUUGAGAAGGAAGAUCAGG | [5036-5056](21/21) |
| 63 | Cross Sp | 21 | UCCUGAUCUUCCUUCUCAACA | UGUUGAGAAGGAAGAUCAGGA | [5035-5055](21/21) |
| 64 | Cross Sp | 21 | AACCUCCUGAUCUUCCUUCUC | GAGAAGGAAGAUCAGGAGGUU | [5031-5051](21/21) |
| 65 | Cross Sp | 21 | AAUGUCAACCUCCUGAUCUUC | GAAGAUCAGGAGGUUGACAUU | [5025-5045](21/21) |
| 66 | Cross Sp | 21 | CCAACAGAAGAAGUUGGAGAC | GUCUCCAACUUCUUCUGUUGG | [4881-4901](21/21) |
| 67 | Cross Sp | 21 | GAAGAAAGAGAAGAUCCAGAC | GUCUGGAUCUUCUCUUUCUUC | [4463-4483](21/21) |
| 68 | Cross Sp | 21 | AGGAGAGACCUUGACUGAAAU | AUUUCAGUCAAGGUCUCUCCU | [4301-4321](21/21) |
| 69 | Cross Sp | 21 | UAAUUGUGGAUGUCAUCCGGU | ACCGGAUGACAUCCACAAUUA | [4273-4293](21/21) |
| 70 | Cross Sp | 21 | AACGUUUAAUUGUGGAUGUCA | UGACAUCCACAAUUAAACGUU | [4267-4287](21/21) |
| 71 | Cross Sp | 21 | AAACGUUUAAUUGUGGAUGUC | GACAUCCACAAUUAAACGUUU | [4266-4286](21/21) |
| 72 | Cross Sp | 21 | CAAAACGUUUAAUUGUGGAUG | CAUCCACAAUUAAACGUUUUG | [4264-4284](21/21) |
| 73 | Cross Sp | 21 | CAUCUUACUGAAUACAAAACG | CGUUUUGUAUUCAGUAAGAUG | [4250-4270](21/21) |
| 74 | Cross Sp | 21 | CCAUCUUACUGAAUACAAAAC | GUUUUGUAUUCAGUAAGAUGG | [4249-4269](21/21) |
| 75 | Cross Sp | 21 | AAAUUCAGACGGUUUUUCCAA | UUGGAAAAACCGUCUGAAUUU | [3882-3092](21/21) |
| 76 | Cross Sp | 21 | AGAAAUUCAUACGGUUUUUCC | GGAAAAACCGUCUGAAUUUCU | [3880-3900](21/21) |
| 77 | Cross Sp | 21 | CCUACCAGAAAUUCAGACGGU | ACCGUCUGAAUUUCUGGUAGG | [3874-3894](21/21) |
| 78 | Cross Sp | 21 | CUCUGUGGACAAAAUCCCUUA | UAAGGGAUUUUGUCCACAGAG | [3554-3574](21/21) |
| 79 | Cross Sp | 21 | AGCUCUGUGGACAAAAUCCCU | AGGGAUUUUGUCCACAGAGCU | [3552-3572](21/21) |
| 80 | Cross Sp | 21 | UCAGCUCUGUGGACAAAAUCC | GGAUUUUGUCCACAGAGCUGA | [3550-3570](21/21) |
| 81 | Cross Sp | 21 | CUUGGGUUAAUCAGAUGGAGU | ACUCCAUCUGAUUAACCCAAG | [3391-3411](21/21) |
| 82 | Cross Sp | 21 | UGAAGGAAAUUAUGGAUGACA | UGUCAUCCAUAAUUUCCUUCA | [3328-3348](21/21) |
| 83 | Cross Sp | 21 | GUGAAGGAAAUUAUGGAUGAC | GUCAUCCAUAAUUUCCUUCAC | [3327-3347](21/21) |
| 84 | Cross Sp | 21 | CUACAAGACUCUCAUCAAUGC | GCAUUGAUGAGAGUCUUGUAG | [2663-2683](21/21) |
| 85 | Cross Sp | 21 | ACUACAAGACUCUCAUCAAUG | CAUUGAUGAGAGUCUUGUAGU | [2662-2682](21/21) |
| 86 | Cross Sp | 21 | GACUACAAGACUCUCAUCAAU | AUUGAUGAGAGUCUUGUAGUC | [2661-2681](21/21) |
| 87 | Cross Sp | 21 | ACAUUAUCAAAAUCCAGGCUU | AAGCCUGGAUUUUGAUAAUGU | [2614-2634](21/21) |
| 88 | Cross Sp | 21 | GACAUUAUCAAAAUCCAGGCU | AGCCUGGAUUUUGAUAAUGUC | [2613-2633](21/21) |
| 89 | Cross Sp | 21 | AGAAGAAGGCAUAUCAAGAUC | GAUCUUGAUAUGCCUUCUUCU | [2473-2493](21/21) |
| 90 | Cross Sp | 21 | CAGAAGAAGGCAUAUCAAGAU | AUCUUGAUAUGCCUUCUUCUG | [2472-2492](21/21) |
| 91 | Cross Sp | 21 | AUUCAGUCACAGUGGAGAGGA | UCCUCUCCACUGUGACUGAAU | [2445-2465](21/21) |
| 92 | Cross Sp | 21 | CCUGCAUUCAGUCACAGUGGA | UCCACUGUGACUGAAUGCAGG | [2440-2460](21/21) |
| 93 | Cross Sp | 21 | ACCUGCAUUCAGUCACAGUGG | CCACUGUGACUGAAUGCAGGU | [2439-2459](21/21) |
| 94 | Cross Sp | 21 | CACCUGCAUUCAGUCACAGUG | CACUGUGACUGAAUUCAGGUG | [2438-2458](21/21) |
| 95 | Cross Sp | 21 | AUCACCUGCAUUCAGUCACAG | CUGUGACUGAAUGCAGGUGAU | [2436-2456](21/21) |
| 96 | Cross Sp | 21 | GUUCGACAGGAAUUCCGAUCC | GGAUCGGAAUUCCUGUCGAAC | [2382-2402](21/21) |
| 97 | Cross Sp | 21 | ACAGCAAGUGGGUGAAGCACU | AGUGCUUCACCCACUUGCUGU | [2146-2166](21/21) |
| 98 | Cross Sp | 21 | AACAGCAAGUGGGUGAAGCAC | GUGCUUCACCCACUUGCUGUU | [2145-2165](21/21) |
| 99 | Cross Sp | 21 | UGGCAGUCCAACAAAGACACC | GGUGUCUUUGUUGGACUGCCA | [1932-1952](21/21) |
| 100 | Cross Sp | 21 | UCUGGCAGUCCAACAAAGACA | UGUCUUUGUUGGACUGCCAGA | [1930-1950](21/21) |
| 101 | Cross Sp | 21 | UGUGGAUGUCAUCCGGUUCCA | UGGAACCGGAUGACAUCCACA | [4277-4297](21/21) |
| 102 | Cross Sp | 21 | AUUGUGGAUGUCAUCCGGUUC | GAACCGGAUGACAUCCACAAU | [4275-4295](21/21) |
| 103 | Cross Sp | 21 | GUUUAAUUGUGGAUGUCAUCC | GGAUGACAUCCACAAUUAAAC | [4270-4290](21/21) |
| 104 | Cross Sp | 21 | UCAACACCCACACUCUCCUGU | ACAGGAGAGUGUGGGUGUUGA | [4009-4029](21/21) |
| 105 | Cross Sp | 21 | UGGUUGGAUGAAAUUCAAGGU | ACCUUGAAUUUCAUCCAACCA | [1905-1925](21/21) |
| 106 | Cross Sp | 21 | AUCAACACCCACACUCUCCUG | CAGGAGAGUGUGGGUGUUUAU | [4008-4028](21/21) |
| 107 | Cross Sp | 21 | CAUCAACACCCACACUCUCCU | AGGAGAGUGUGGGUGUUGAUG | [4007-4027](21/21) |
| 108 | Cross Sp | 21 | UCAUCAACACCCACACUCUCC | GGAGAGUGUGGGUGUUGAUGA | [4006-4026](21/21) |
| 109 | Cross Sp | 21 | AUCAUCAACACCCACACUCUC | GAGAGUGUGGGUGUUGAUGAU | [4005-4025](21/21) |
| 110 | Cross Sp | 21 | ACCAGAAAUUCAGACGGUUUU | AAAACCGUCUGAAUUUCUGGU | [3877-3897](21/21) |
| 111 | Cross Sp | 21 | UACCAGAAAUUCAGACGGUUU | AAACCGUCUGAAUUUCUGGUA | [3876-3896](21/21) |
| 112 | Cross Sp | 21 | GUCCUACCAGAAAUUCAGACG | CGUCUGAAUUUCUGGUAGGAC | [3872-3892](21/21) |
| 113 | Cross Sp | 21 | CAGCUCUGUGGACAAAAUCCC | GGGAUUUUGUCCACAGAGCUG | [3551-3571](21/21) |
| 114 | Cross Sp | 21 | AUGACUACAAGACUCUCAUCA | UGAUGAGAGUCUUGUAGUCAU | [2659-2679](21/21) |
| 115 | Cross Sp | 21 | CAUUAUCAAAAUCCAGGCUUU | AAAGCCUGGAUUUUGAUAAUG | [2615-2635](21/21) |
| 116 | Cross Sp | 21 | UGACAUUAUCAAAAUCCAGGC | GCCUGGAUUUUGAUAAUGUCA | [2612-2632](21/21) |
| 117 | Cross Sp | 21 | AUUAAUGAAGCCCUGGAUGAA | UUCAUCCAGGGCUUCAUUAAU | [1734-1754](21/21) |
| 118 | Cross Sp | 21 | GAAUCUGGCAGUCCAACAAAG | CUUUGUUGGACUGCCAGAUUC | [1927-1947](21/21) |
| 119 | Cross Sp | 21 | UGGAAUCUGGCAGUCCAACAA | UUGUUGGACUGCCAGAUUCCA | [1925-1945](21/21) |
| 120 | Cross Sp | 21 | GUGGAAUCUGGCAGUCCAACA | UGUUGGACUGCCAGAUUCCAC | [1924-1944](21/21) |
| 121 | Cross Sp | 21 | UCAAGGUGGAAUCUGGCAGUC | GACUGCCAGAUUCCACCUUGA | [1919-1939](21/21) |
| 122 | Cross Sp | 21 | UUCAAGGUGGAAUCUGGCAGU | ACUGCCAGAUUCCACCUUGAA | [1918-1938](21/21) |
| 123 | Cross Sp | 21 | AAAUUCAAGGUGGAAUCUGGC | GCCAGAUUCCACCUUGAAUUU | [1915-1935](21/21) |
| 124 | Cross Sp | 21 | UGAAAUUCAAGGUGGAAUCUG | CAGAUUCCACCUUGAAUUUCA | [1913-1933](21/21) |
| 125 | Cross Sp | 21 | GAUGAAAUUCAAGGUGGAAUC | GAUUCCACCUUGAAUUUCAUC | [1911-1931](21/21) |
| 126 | Cross Sp | 21 | UUGGAUGAAAUUCAAGGUGGA | UCCACCUUGAAUUUCAUCCAA | [1908-1928](21/21) |
| 127 | Cross Sp | 21 | GCUGCAGCUACAGUAUGAAGG | CCUUCAUACUGUAGCUGCAGC | [4967-4987](21/21) |
| 128 | Cross Sp | 21 | UGCUGCAGCUACAGUAUGAAG | CUUCAUACUGUAGCUGCAGCA | [4966-4986](21/21) |
| 129 | Cross Sp | 21 | GUUGGAUGAAAUUCAAGGUGG | CCACCUUGAAGUUUCAUCCAAC | [1907-1927](21/21) |
| 130 | Cross Sp | 21 | UUAAUUGUGGAUGUCAUCCGG | CCGGAUGACAUCCACAAUUAA | [4272-4292](21/21) |
| 131 | Cross Sp | 21 | ACGUUUAAUUGUGGAUGUCAU | AUGACAUCCACAAUUAAACGU | [4268-4288](21/21) |
| 132 | Cross Sp | 21 | AACACCCACACUCUCCUGUUG | CAACAGGAGAGUGUGGGUGUU | [4011-4031](21/21) |
| 133 | Cross Sp | 21 | CAGAAAUUCAGACGGUUUUUC | GAAAAACCGUCUGAAUUUCUG | [3879-3899](21/21) |
| 134 | Cross Sp | 21 | UCCUACCAGAAAUUCAGACGG | CCGUCUGAAUUUCUGGUAGGA | [3873-3893](21/21) |
| 135 | Cross Sp | 21 | AGUCCUACCAGAAAUUCAGAC | GUCUGAAUUUCUGGUAGGACU | [3871-3891](21/21) |
| 136 | Cross Sp | 21 | UUAAUGAAGCCCUGGAUGAAG | CUUCAUCCAGGGCUUCAUUAA | [1735-1755](21/21) |
| 137 | Cross Sp | 21 | AAUAACAGCAAGUGGGUGAAG | CUUCACCCACUUGCUGUUAUU | [2142-2162](21/21) |
| 138 | Cross Sp | 21 | AAUUCAAGGUGGAAUCUGGCA | UGCCAGAUUCCACCUUGAAUU | [1916-1936](21/21) |
| 139 | Cross Sp | 21 | GAAAUUCAAGGUGGAAUCUGG | CCAGAUUCCACCUUGAAUUUC | [1914-1934](21/21) |
| 140 | Cross Sp | 21 | AACAGAAGAAGUUGGAGACUU | AAGUCUCCAACUUCUUCUGUU | [4883-4903](21/21) |
| 141 | Cross Sp | 21 | GAAUGCCAUGGAUGAGAUUGG | CCAAUCUCAUCCAUGGCAUUC | [461-481](21/21) |
| 142 | Cross Sp | 21 | UUGUGGAUGUCAUCCGGUUCC | GGAACCGGAUGACAUCCACAA | [4276-4296](21/21) |
| 143 | Cross Sp | 21 | AAUUGUGGAUGUCAUCCGGUU | AACCGGAUGACAUCCACAAUU | [4274-4294](21/21) |
| 144 | Cross Sp | 21 | UAAUGAAGCCCUGGAUGAAGG | CCUUCAUCCAGGGCUUCAUUA | [1736-1756](21/21) |
| 145 | Cross Sp | 21 | AUGACAUUAUCAAAAUCCAGG | CCUGGAUUUUGAUAAUGUCAU | [2611-2631](21/21) |
| 146 | Cross Sp | 21 | AUAACAGCAAGUGGGUGAAGC | GCUUCACCCACUUGCUGUUAU | [2143-2163](21/21) |
| 147 | Cross Sp | 21 | AAGGUGGAAUCUGGCAGUCCA | UGGACUGCCAGAUUCCACCUU | [1921-1941](21/21) |
| 148 | Cross Sp | 21 | AUUCAAGGUGGAAUCUGGCAG | CUGCCAGAUUCCACCUUGAAU | [1917-1937](21/21) |
| 149 | Cross Sp | 21 | UCCAACAGAAGAAGUUGGAGA | UCUCCAACUUCUUCUGUUGGA | [4880-4900](21/21) |
| 150 | Cross Sp | 21 | UUUAAUUGUGGAUGUCAUCCG | CGGAUGACAUCCACAAUUAAA | [4271-4291](21/21) |
| 151 | Cross Sp | 21 | ACACCCACACUCUCCUGUUGG | CCAACAGGAGAGUGUGGGUGU | [4012-4032](21/21) |
| 152 | Cross Sp | 21 | CAAGUGGGUGAAGCACUGGGU | ACCCAGUGCUUCACCCACUUG | [2150-2170](21/21) |
| 153 | Cross Sp | 21 | GUGGAUGUCAUCCGGUUCCAG | CUGGAACCGGAUGACAUCCAC | [4278-4298](21/21) |
| 154 | Cross Sp | 21 | GGUGGAAUCUGGCAGUCCAAC | GUUGGACUGCCAGAUUCCACC | [1923-1943](21/21) |
| 155 | Cross Sp | 21 | CAAGGUGGAAUCUGGCAGUCC | GGACUGCCAGAUUCCACCUUG | [1920-1940](21/21) |
| 156 | Cross Sp | 21 | GAUGUCAUCCGGUUCCAGCCA | UGGCUGGAACCGGAUGACAUC | [4281-4301](21/21) |
| 157 | Cross Sp | 21 | CACCCACACUCUCCUGUUGGA | UCCAACAGGAGAGUGUGGGUG | [4013-4033](21/21) |
| 158 | Cross Sp | 21 | AGCAAGUGGGUGAAGCACUGG | CCAGUGCUUCACCCACUUGCU | [2148-2168](21/21) |
| 159 | Cross Sp | 21 | CAGCAAGUGGGUGAAGCACUG | CAGUGCUUCACCCACUUGCUG | [2147-2167](21/21) |
| 160 | Cross Sp | 21 | UGGAUGUCAUCCGGUUCCAGC | GCUGGAACCGGAUGACAUCCA | [4279-4299](21/21) |
| 161 | Cross Sp | 21 | AUGUCAUCCGGUUCCAGCCAG | CUGGCUGGAACCGGAUGACAU | [4282-4302](21/21) |
| 162 | Human | 21 | GCAAAUAUCGACCUGGCUUUA | UAAAGCCAGGUCGAUAUUUGC | [1029-1049](21/21) |
| 163 | Human | 21 | GGAUGAAAGGAGACGUCAGAA | UUCUGACGUCUCCUUUCAUCC | [194-214](21/21) |
| 164 | Human | 21 | GGACAAAAUGACAAAUGCUAA | UUAGCAUUUGUCAUUUUGUCC | [902-922](21/21) |
| 165 | Human | 21 | GCAUCCACUUACCAGGAUAUA | UAUAUCCUGGUAAGUGGAUGC | [864-884](21/21) |
| 166 | Human | 21 | CGACCAGCCAUCAUGACAUUU | AAAUGUCAUGAUGGCUGGUCG | [7084-7104](21/21) |
| 167 | Human | 21 | GUAGAAUUCCAGCCGACACAU | AUGUGUCGGCUGGAAUUCUAC | [790-810](21/21) |
| 168 | Human | 21 | CAACCCUGUUUCCUUAAAAGA | UCUUUUAAGGAAACAGGGUUG | [6766-6786](21/21) |
| 169 | Human | 21 | CAACUAGCAUUGCCUCAAAAA | UUUUUGAGGCAAUGCUAGUUG | [6724-6744](21/21) |
| 170 | Human | 21 | GGAAUGGGUUAGCCCUAAUGU | ACAUUAGGGCUAACCCAUUCC | [5890-5910](21/21) |
| 171 | Human | 21 | GAAGUGACAGUCAUGUUCAAA | UUUGAACAUGACUGUCACUUC | [5648-5668](21/21) |
| 172 | Human | 21 | GGAAGCUAUUAGCACCAAUGA | UCAUUGGUGCUAAUAGCUUCC | [5458-5478](21/21) |
| 173 | Human | 21 | GGAAAGGUUGACUUCACAGAA | UUCUGUGAAGUCAACCUUUCC | [618-638](21/21) |
| 174 | Human | 21 | CAACAGCACCUCAAUCUGAUA | UAUCAGAUUGAGGUGCUGUUG | [5175-5195](21/21) |
| 175 | Human | 21 | GCAAAGACCUAGCCAACAACA | UGUUGUUGGCUAGGUCUUUGC | [5159-5179](21/21) |
| 176 | Human | 21 | GGAGUUGCAGUCAUGAAAUUA | UAAUUUCAUGACUGCAACUCC | [4986-5006](21/21) |
| 177 | Human | 21 | GGAGUUCAAAUGGAGACUUUU | AAAAGUCUCCAUUUGAACUCC | [4926-4946](21/21) |
| 178 | Human | 21 | GCAAGGGCAAAGUCUCCAAAA | UUUUGGAGACUUUGCCCUUGC | [4720-4740](21/21) |
| 179 | Human | 21 | GCAACCUCACUCUUCAAGAGA | UCUCUUGAAGAGUGAGGUUGC | [4444-4464](21/21) |
| 180 | Human | 21 | GAAGACAGCAACCUCACUCUU | AAGAGUGAGGUUGCUGUCUUC | [4437-4457](21/21) |
| 181 | Human | 21 | CCAAAACACCUGACAAGAUGA | UCAUCUUGUCAGGUGUUUUGG | [4396-4416](21/21) |
| 182 | Human | 21 | GAACAUGCCAAGAUGUAUCUA | UAGAUACAUCUUGGCAUGUUC | [530-550](21/21) |
| 183 | Human | 21 | CGAAAGAACAUGCCAAGAUGU | ACAUCUUGGCAUGUUCUUUCG | [525-545](21/21) |
| 184 | Human | 21 | GGAUGCUCGAACCAUCUUACU | AGUAAGAUGGUUCGAGCAUCC | [4238-4258](21/21) |
| 185 | Human | 21 | CCAGUAAUCUACAUUUCCAUU | AAUGGAAAUGUAGAUUACUGG | [3978-3998](21/21) |
| 186 | Human | 21 | CCAGAGCUUCAGGAUAAAUUU | AAAUUUAUCCUGAAGCUCUGG | [3918-3938](21/21) |
| 187 | Human | 21 | GCAUGCUGCUUCCAAUAAGAU | AUCUUAUUGGAAGCAGCAUGC | [3800-3820](21/21) |
| 188 | Human | 21 | GCAAGCAAACUGCCCUAUGAU | AUCAUAGGGCAGUUUGCUUGC | [3426-3446](21/21) |
| 189 | Human | 21 | GGAUGACAAAUCUCUCAACAU | AUGUUGAGAGAUUUGUCAUCC | [3341-3361](21/21) |
| 190 | Human | 21 | CAAGAGAUUGUGACAGGAAAU | AUUUCCUGUCACAAUCUCUUG | [3225-3245](21/21) |
| 191 | Human | 21 | GAACAAGUCCACCAAGUUCAU | AUGAACUUGGUGGACUUGUUC | [3086-3106](21/21) |
| 192 | Human | 21 | CCAGAACAAGUCCACCAAGUU | AACUUGGUGGACUUGUUCUGG | [3083-3103](21/21) |
| 193 | Human | 21 | GAAGCUUACCAGCACCUGUUU | AAACAGGUGCUGGUAAGCUUC | [3009-3029](21/21) |
| 194 | Human | 21 | GGAUGUGGUUUCCCACAGUAA | UUACUGUGGGAAACCACAUCC | [2885-2905](21/21) |
| 195 | Human | 21 | GAAGAGGUUAUCACCCUCAUU | AAUGAGGGUGAUAACCUCUUC | [2781-2801](21/21) |
| 196 | Human | 21 | GAACUUCUUCUCUCCCAAAGU | ACUUUGGGAGAGAAGAAGUUC | [347-367](21/21) |
| 197 | Human | 21 | GGAAUUCCGAUCCAGGAUGAA | UUCAUCCUGGAUCGGAAUUCC | [2390-2410](21/21) |
| 198 | Human | 21 | GAAGUGGCCCAGCAUUACCAA | UUGGUAAUGCUGGGCCACUUC | [1821-1841](21/21) |
| 199 | Human | 21 | GCACAUGCAGAGAAUAAUGAA | UUCAUUAUUCUCUGCAUGUGC | [1623-1643](21/21) |
| 200 | Human | 21 | GAAGCAAUUGAGCAGUUCAGU | ACUGAACUGCUCAAUUGCUUC | [1529-1549](21/21) |
| 201 | Human | 21 | GCAGCGACAAAGUCCUGAACA | UGUUCAGGACUUUGUCGCUGC | [1409-1429](21/21) |
| 202 | Human | 21 | GCAGUAGCACUGAUUAAUGCU | AGCAUUAAUCAGUGCUACUGC | [1272-1292](21/21) |
| 203 | Human | 21 | CGAGGACUGCAGCAACAGAAU | AUUCUGUUGCUGCAGUCCUCG | [1107-1127](21/21) |
| 204 | Human | 21 | GCAGAGGAGAUGGAUGAAAGG | CCUUUCAUCCAUCUCCUCUGC | [183-203](21/21) |
| 205 | Human | 21 | CAGGACAAAAUGACAAAUGCU | AGCAUUUGUCAUUUUGUCCUG | [900-920](21/21) |
| 206 | Human | 21 | GCUAAGCAGGACAAAAUGACA | UGUCAUUUUGUCCUGCUUAGC | [894-914](21/21) |
| 207 | Human | 21 | CCACUUACCAGGAUAUACUUU | AAAGUAUAUCCUGGUAAGUGG | [868-888](21/21) |
| 208 | Human | 21 | CCGAAUGCCAUGCUUGUAAAU | AUUUACAAGCAUGGCAUUCGG | [828-848](21/21) |
| 209 | Human | 21 | ACACAUUUGCAGCUUUGAAAA | UUUUCAAAGCUGCAAAUGUGU | [805-825](21/21) |
| 210 | Human | 21 | AGAUUGUUGCUGCACAUAGUU | AACUAUGUGCAGCAACAAUCU | [7146-7166](21/21) |
| 211 | Human | 21 | GUCAGAUUGUUGCUGCACAUA | UAUGUGCAGCAACAAUCUGAC | [7143-7163](21/21) |
| 212 | Human | 21 | CCGACACAUUUGCAGCUUUGA | UCAAAGCUGCAAAUGUGUCGG | [802-822](21/21) |
| 213 | Human | 21 | CCAUGAAUUUACUUCCUCCCA | UGGGAGGAAGUAAAUUCAUGG | [7106-7126](21/21) |
| 214 | Human | 21 | CCAGCCAUCAUGACAUUUACC | GGUAAAUGUCAUGAUGGCUGG | [7087-7107](21/21) |
| 215 | Human | 21 | CUCCUCUCUCUCCUCCUUUGA | UCAAAGGAGGAGAGAGAGGAG | [7054-7074](21/21) |
| 216 | Human | 21 | GUCUCCUCUCUCUCCUCCUUU | AAAGGAGGAGAGAGAGGAGAC | [7052-7072](21/21) |
| 217 | Human | 21 | GCAUUCUCUGUCUCCUCUCUC | GAGAGAGGAGACAGAGAAUGC | [7043-7063](21/21) |
| 218 | Human | 21 | CACACAGUUCUCAUUACUGUU | AACAGUAAUGAGAACUGUGUG | [7008-7028](21/21) |
| 219 | Human | 21 | GAAACCACACAGUUCUCAUUA | UAAUGAGAACUGUGUGGUUUC | [7003-7023](21/21) |
| 220 | Human | 21 | GGAUGCCCCAACAAACUCAUG | CAUGAGUUUGUUGGGGCAUCC | [6977-6997](21/21) |
| 221 | Human | 21 | CAAUUGCUAAUGCAGAAUAGU | ACUAUUCUGCAUUAGCAAUUG | [6894-6914](21/21) |
| 222 | Human | 21 | GAAGCUAUUGACCGUAGAAUU | AAUUCUACGGUCAAUAGCUUC | [777-797](21/21) |
| 223 | Human | 21 | CAGAGUUUACAGCUCCUUUAA | UUAAAGGAGCUGUAAACUCUG | [6831-6851](21/21) |
| 224 | Human | 21 | GGCAAUAAUGAGCAGAGUUUA | UAAACUCUGCUCAUUAUUGCC | [6819-6839](21/21) |
| 225 | Human | 21 | GUGUCAACCCUGUUUCCUUAA | UUAAGGAAACAGGGUUGACAC | [6762-6782](21/21) |
| 226 | Human | 21 | CCAAAGUGUGUCAACCCUGUU | AACAGGGUUGACACACUUUGG | [6755-6775](21/21) |
| 227 | Human | 21 | GGACCAACCAAAGUGUGUCAA | UUGACACACUUUGGUUGGUCC | [6748-6768](21/21) |
| 228 | Human | 21 | UGAUGCUAUUACAACUAGCAU | AUGCUAGUUGUAAUAGCAUCA | [6713-6733](21/21) |
| 229 | Human | 21 | GGAAGACAGGUUUUUUAAAGA | UCUUUAAAAAACCUGUCUUCC | [6669-6689](21/21) |
| 230 | Human | 21 | GUAUUUUGGAAGACAGGUUUU | AAAACCUGUCUUCCAAAAUAC | [6662-6682](21/21) |
| 231 | Human | 21 | CCAGACCUUGUUUUUUGUUGU | ACAACAAAAAACAAGGUCUGG | [6643-6663](21/21) |
| 232 | Human | 21 | CACUACCCAGACCUUGUUUUU | AAAAACAAGGUCUGGGUAGUG | [6637-6657](21/21) |
| 233 | Human | 21 | GCCACUACCCAGACCUUGUUU | AAACAAGGUCUGGGUAGUGGC | [6635-6655](21/21) |
| 234 | Human | 21 | CCGCAUUACAUGCUGCUGUUA | UAACAGCAGCAUGUAAUGCGG | [745-765](21/21) |
| 235 | Human | 21 | GUAGUGUGUCCCUAUUAUAAA | UUUAUAAUAGGGACACACUAC | [6527-6547](21/21) |
| 236 | Human | 21 | CUAUGUAGUGUGUCCCUAUUA | UAAUAGGGACACACUACAUAG | [6523-6543](21/21) |
| 237 | Human | 21 | GCCAAUAAACUGCUUUAAUGA | UCAUUAAAGCAGUUUAUUGGC | [6494-6514](21/21) |
| 238 | Human | 21 | GCCUCUAUUUUCUUUCUUUGA | UCAAAGAAAGAAAAUAGAGGC | [6408-6428](21/21) |
| 239 | Human | 21 | CUGCUGAGCCUCUAUUUUCUU | AAGAAAAUAGAGGCUCAGCAG | [6401-6421](21/21) |
| 240 | Human | 21 | CAUGCUGCUGAGCCUCUAUUU | AAAUAGAGGCUCAGCAGCAUG | [6397-6417](21/21) |
| 241 | Human | 21 | GGAUAAUGAAAGACUUACUGC | GCAGUAAGUCUUUCAUUAUCC | [164-184](21/21) |
| 242 | Human | 21 | AGAAAGCUUUUGUUCUUGGAU | AUCCAAGAACAAAAGCUUUCU | [6319-6339](21/21) |
| 243 | Human | 21 | GCAUUUAGAAAGCUUUUGUUC | GAACAAAAGCUUUCUAAAUGC | [6313-6333](21/21) |
| 244 | Human | 21 | CCAGUUUUCUGGGCUUAGUUU | AAACUAAGCCCAGAAAACUGG | [6267-6287](21/21) |
| 245 | Human | 21 | CCACAUUGUGCCUUUAUUUUA | UAAAAUAAAGGCACAAUGUGG | [6240-6260](21/21) |
| 246 | Human | 21 | GCCCACAUUGUGCCUUUAUUU | AAAUAAAGGCACAAUGUGGGC | [6238-6258](21/21) |
| 247 | Human | 21 | GCAUCUUGGCUAAUGAACUGU | ACAGUUCAUUAGCCAAGAUGC | [712-732](21/21) |
| 248 | Human | 21 | CCCCAUUUUUUCUCCUUUUUU | AAAAAAGGAGAAAAAAUGGGG | [6209-6229](21/21) |
| 249 | Human | 21 | GGGGGCAUCUUGGCUAAUGAA | UUCAUUAGCCAAGAUGCCCCC | [708-728](21/21) |
| 250 | Human | 21 | CCUAUGUUACACGUGAGAUUA | UAAUCUCACGUGUAACAUAGG | [6175-6195](21/21) |
| 251 | Human | 21 | AAACAAAACCAGCCAACCUAU | AUAGGUUGGCUGGUUUUGUUU | [6159-6179](21/21) |
| 252 | Human | 21 | GCAGUGUUCAGGGAGAUAAUU | AAUUAUCUCCCUGAACACUGC | [6115-6135](21/21) |
| 253 | Human | 21 | CUAGGAUUUAUCUGCAGUGUU | AACACUGCAGAUAAAUCCUAG | [6102-6122](21/21) |
| 254 | Human | 21 | GAACUCUUGAAACUUAUAGCU | AGCUAUAAGUUUCAAGAGUUC | [6075-6095](21/21) |
| 255 | Human | 21 | GCAUCAAAAGCCUUAGAAUAA | UUAUUCUAAGGCUUUUGAUGC | [6018-6038](21/21) |
| 256 | Human | 21 | CCAGAUGCCUGCCUUUAGCAA | UUGCUAAAGGCAGGCAUCUGG | [683-703](21/21) |
| 257 | Human | 21 | GCCCUAAUGUAGAAUGUCAUU | AAUGACAUUCUACAUUAGGGC | [5901-5921](21/21) |
| 258 | Human | 21 | GCAUCCAGAUGCCUGCCUUUA | UAAAGGCAGGCAUCUGGAUGC | [679-699](21/21) |
| 259 | Human | 21 | CCAUCCCUAUGGCAGAGGAAU | AUUCCUCUGCCAUAGGGAUGG | [5874-5894](21/21) |

| | | | | |
|---|---|---|---|---|
| | | Oligo | | |
| No | Source | Length | mouse 7710041 | rat 34857387 |

FOR 19
STRETCHES:

| | | | | |
|---|---|---|---|---|
| 1 | Cross Sp | 19 | [1884-1902](19/19) | [1929-1947](19/19) |
| 2 | Cross Sp | 19 | [4247-4265](19/19) | [4292-4310](19/19) |
| 3 | Cross Sp | 19 | [2661-2679](19/19) | [2706-2724](19/19) |
| 4 | Cross Sp | 19 | [1925-1943](19/19) | [1970-1988](19/19) |
| 5 | Cross Sp | 19 | [458-476](19/19) | [503-521](19/19) |
| 6 | Cross Sp | 19 | [1908-1926](19/19) | [1953-1971](19/19) |
| 7 | Cross Sp | 19 | [5037-5055](19/19) | [5082-5100](19/19) |
| 8 | Cross Sp | 19 | [4766-4784](19/19) | [4811-4829](19/19) |
| 9 | Cross Sp | 19 | [4301-4319](19/19) | [4346-4364](19/19) |
| 10 | Cross Sp | 19 | [4300-4318](19/19) | [4345-4363](19/19) |
| 11 | Cross Sp | 19 | [3877-3895](19/19) | [3922-3940](19/19) |
| 12 | Cross Sp | 19 | [3548-3566](19/19) | [3593-3611](19/19) |
| 13 | Cross Sp | 19 | [2659-2677](19/19) | [2704-2722](19/19) |
| 14 | Cross Sp | 19 | [637-655](19/19) | [682-700](19/19) |
| 15 | Cross Sp | 19 | [563-581](19/19) | [608-626](19/19) |
| 16 | Cross Sp | 19 | [2472-2490](19/19) | [2517-2535](19/19) |
| 17 | Cross Sp | 19 | [2431-2449](19/19) | [2476-2494](19/19) |
| 18 | Cross Sp | 19 | [2101-2119](19/19) | [2146-2164](19/19) |
| 19 | Cross Sp | 19 | [459-477](19/19) | [504-522](19/19) |
| 20 | Cross Sp | 19 | [1931-1949](19/19) | [1976-1994](19/19) |
| 21 | Cross Sp | 19 | [1918-1936](19/19) | [1963-1981](19/19) |
| 22 | Cross Sp | 19 | [1911-1929](19/19) | [1956-1974](19/19) |
| 23 | Cross Sp | 19 | [1902-1920](19/19) | [1947-1965](19/19) |
| 24 | Cross Sp | 19 | [1898-1916](19/19) | [1943-1961](19/19) |
| 25 | Cross Sp | 19 | [255-273](19/19) | [300-318](19/19) |
| 26 | Cross Sp | 19 | [1894-1912](19/19) | [1939-1957](19/19) |
| 27 | Cross Sp | 19 | [1889-1907](19/19) | [1934-1952](19/19) |
| 28 | Cross Sp | 19 | [5044-5062](19/19) | [5089-5107](19/19) |
| 29 | Cross Sp | 19 | [5042-5060](19/19) | [5087-5105](19/19) |
| 30 | Cross Sp | 19 | [5039-5057](19/19) | [5084-5102](19/19) |
| 31 | Cross Sp | 19 | [5036-5054](19/19) | [5081-5099](19/19) |
| 32 | Cross Sp | 19 | [5033-5051](19/19) | [5078-5096](19/19) |
| 33 | Cross Sp | 19 | [5032-5050](19/19) | [5077-5095](19/19) |
| 34 | Cross Sp | 19 | [5028-5046](19/19) | [5073-5091](19/19) |
| 35 | Cross Sp | 19 | [5027-5045](19/19) | [5072-5090](19/19) |
| 36 | Cross Sp | 19 | [5024-5042](19/19) | [5069-5087](19/19) |
| 37 | Cross Sp | 19 | [4965-4983](19/19) | [5010-5028](19/19) |
| 38 | Cross Sp | 19 | [1352-1370](19/19) | [1397-1415](19/19) |
| 39 | Cross Sp | 19 | [4880-4898](19/19) | [4925-4943](19/19) |
| 40 | Cross Sp | 19 | [4460-4478](19/19) | [4505-4523](19/19) |
| 41 | Cross Sp | 19 | [4268-4286](19/19) | [4313-4331](19/19) |
| 42 | Cross Sp | 19 | [4007-4025](19/19) | [4052-4070](19/19) |
| 43 | Cross Sp | 19 | [3551-3569](19/19) | [3596-3614](19/19) |
| 44 | Cross Sp | 19 | [640-658](19/19) | [685-703](19/19) |
| 45 | Cross Sp | 19 | [3329-3347](19/19) | [3374-3392](19/19) |
| 46 | Cross Sp | 19 | [2666-2684](19/19) | [2711-2729](19/19) |
| 47 | Cross Sp | 19 | [2664-2682](19/19) | [2709-2727](19/19) |
| 48 | Cross Sp | 19 | [2663-2681](19/19) | [2708-2726](19/19) |
| 49 | Cross Sp | 19 | [2657-2675](19/19) | [2702-2720](19/19) |
| 50 | Cross Sp | 19 | [2475-2493](19/19) | [2520-2538](19/19) |
| 51 | Cross Sp | 19 | [2449-2467](19/19) | [2494-2512](19/19) |
| 52 | Cross Sp | 19 | [2446-2464](19/19) | [2491-2509](19/19) |
| 53 | Cross Sp | 19 | [562-580](19/19) | [607-625](19/19) |
| 54 | Cross Sp | 19 | [2438-2456](19/19) | [2483-2501](19/19) |
| 55 | Cross Sp | 19 | [2432-2450](19/19) | [2477-2495](19/19) |
| 56 | Cross Sp | 19 | [251-269](19/19) | [296-314](19/19) |
| 57 | Cross Sp | 19 | [2144-2162](19/19) | [2189-2207](19/19) |
| 58 | Cross Sp | 19 | [2102-2120](19/19) | [2147-2165](19/19) |
| 59 | Cross Sp | 19 | [1930-1948](19/19) | [1975-1993](19/19) |
| 60 | Cross Sp | 19 | [1928-1946](19/19) | [1973-1991](19/19) |
| 61 | Cross Sp | 19 | [1927-1945](19/19) | [1972-1990](19/19) |
| 62 | Cross Sp | 19 | [1924-1942](19/19) | [1969-1987](19/19) |
| 63 | Cross Sp | 19 | [1922-1940](19/19) | [1967-1985](19/19) |
| 64 | Cross Sp | 19 | [1912-1930](19/19) | [1957-1975](19/19) |
| 65 | Cross Sp | 19 | [1905-1923](19/19) | [1950-1968](19/19) |
| 66 | Cross Sp | 19 | [1904-1922](19/19) | [1949-1967](19/19) |
| 67 | Cross Sp | 19 | [1901-1919](19/19) | [1946-1964](19/19) |
| 68 | Cross Sp | 19 | [1899-1917](19/19) | [1944-1962](19/19) |
| 69 | Cross Sp | 19 | [1897-1915](19/19) | [1942-1960](19/19) |
| 70 | Cross Sp | 19 | [1895-1913](19/19) | [1940-1958](19/19) |
| 71 | Cross Sp | 19 | [1888-1906](19/19) | [1933-1951](19/19) |
| 72 | Cross Sp | 19 | [1887-1905](19/19) | [1932-1950](19/19) |
| 73 | Cross Sp | 19 | [1885-1903](19/19) | [1930-1948](19/19) |
| 74 | Cross Sp | 19 | [1882-1900](19/19) | [1927-1945](19/19) |
| 75 | Cross Sp | 19 | [1736-1754](19/19) | [1781-1799](19/19) |
| 76 | Cross Sp | 19 | [1355-1373](19/19) | [1400-1418](19/19) |
| 77 | Cross Sp | 19 | [5041-5059](19/19) | [5086-5104](19/19) |
| 78 | Cross Sp | 19 | [1354-1372](19/19) | [1399-1417](19/19) |
| 79 | Cross Sp | 19 | [5035-5053](19/19) | [5080-5098](19/19) |
| 80 | Cross Sp | 19 | [5034-5052](19/19) | [5079-5097](19/19) |
| 81 | Cross Sp | 19 | [5030-5048](19/19) | [5075-5093](19/19) |
| 82 | Cross Sp | 19 | [1353-1371](19/19) | [1398-1416](19/19) |
| 83 | Cross Sp | 19 | [4967-4985](19/19) | [5012-5030](19/19) |
| 84 | Cross Sp | 19 | [4964-4982](19/19) | [5009-5027](19/19) |
| 85 | Cross Sp | 19 | [4883-4901](19/19) | [4928-4946](19/19) |
| 86 | Cross Sp | 19 | [4878-4896](19/19) | [4923-4941](19/19) |
| 87 | Cross Sp | 19 | [4877-4895](19/19) | [4922-4940](19/19) |
| 88 | Cross Sp | 19 | [4463-4481](19/19) | [4508-4526](19/19) |
| 89 | Cross Sp | 19 | [4298-4316](19/19) | [4343-4361](19/19) |
| 90 | Cross Sp | 19 | [4274-4292](19/19) | [4319-4337](19/19) |
| 91 | Cross Sp | 19 | [4262-4280](19/19) | [4307-4325](19/19) |
| 92 | Cross Sp | 19 | [4261-4279](19/19) | [4306-4324](19/19) |
| 93 | Cross Sp | 19 | [4251-4269](19/19) | [4296-4314](19/19) |
| 94 | Cross Sp | 19 | [4009-4027](19/19) | [4054-4072](19/19) |
| 95 | Cross Sp | 19 | [4005-4023](19/19) | [4050-4068](19/19) |
| 96 | Cross Sp | 19 | [4004-4022](19/19) | [4049-4067](19/19) |
| 97 | Cross Sp | 19 | [4003-4021](19/19) | [4048-4066](19/19) |
| 98 | Cross Sp | 19 | [3879-3897](19/19) | [3924-3942](19/19) |
| 99 | Cross Sp | 19 | [3876-3894](19/19) | [3921-3939](19/19) |
| 100 | Cross Sp | 19 | [3873-3891](19/19) | [3918-3936](19/19) |
| 101 | Cross Sp | 19 | [3870-3888](19/19) | [3915-3933](19/19) |
| 102 | Cross Sp | 19 | [3869-3887](19/19) | [3914-3932](19/19) |
| 103 | Cross Sp | 19 | [3868-3886](19/19) | [3913-3931](19/19) |
| 104 | Cross Sp | 19 | [3549-3567](19/19) | [3594-3612](19/19) |
| 105 | Cross Sp | 19 | [2879-2897](19/19) | [2924-2942](19/19) |
| 106 | Cross Sp | 19 | [2656-2674](19/19) | [2701-2719](19/19) |
| 107 | Cross Sp | 19 | [2615-2633](19/19) | [2660-2678](19/19) |
| 108 | Cross Sp | 19 | [2435-2453](19/19) | [2480-2498](19/19) |
| 109 | Cross Sp | 19 | [3878-3896](19/19) | [3923-3941](19/19) |
| 110 | Cross Sp | 19 | [3875-3893](19/19) | [3920-3938](19/19) |
| 111 | Cross Sp | 19 | [3874-3892](19/19) | [3919-3937](19/19) |
| 112 | Cross Sp | 19 | [3872-3890](19/19) | [3917-3935](19/19) |
| 113 | Cross Sp | 19 | [3871-3889](19/19) | [3916-3934](19/19) |
| 114 | Cross Sp | 19 | [3552-3570](19/19) | [3597-3615](19/19) |
| 115 | Cross Sp | 19 | [2660-2678](19/19) | [2705-2723](19/19) |
| 116 | Cross Sp | 19 | [2658-2676](19/19) | [2703-2721](19/19) |
| 117 | Cross Sp | 19 | [2613-2631](19/19) | [2658-2676](19/19) |
| 118 | Cross Sp | 19 | [2612-2630](19/19) | [2657-2675](19/19) |
| 119 | Cross Sp | 19 | [2611-2629](19/19) | [2656-2674](19/19) |
| 120 | Cross Sp | 19 | [2610-2628](19/19) | [2655-2673](19/19) |
| 121 | Cross Sp | 19 | [2434-2452](19/19) | [2479-2497](19/19) |
| 122 | Cross Sp | 19 | [2433-2451](19/19) | [2478-2496](19/19) |
| 123 | Cross Sp | 19 | [2380-2398](19/19) | [2425-2443](19/19) |
| 124 | Cross Sp | 19 | [461-479](19/19) | [506-524](19/19) |
| 125 | Cross Sp | 19 | [2143-2161](19/19) | [2188-2206](19/19) |
| 126 | Cross Sp | 19 | [2141-2159](19/19) | [2186-2204](19/19) |
| 127 | Cross Sp | 19 | [1926-1944](19/19) | [1971-1989](19/19) |
| 128 | Cross Sp | 19 | [1917-1935](19/19) | [1962-1980](19/19) |
| 129 | Cross Sp | 19 | [1914-1932](19/19) | [1959-1977](19/19) |
| 130 | Cross Sp | 19 | [1910-1928](19/19) | [1955-1973](19/19) |
| 131 | Cross Sp | 19 | [1906-1924](19/19) | [1951-1969](19/19) |
| 132 | Cross Sp | 19 | [1734-1752](19/19) | [1779-1797](19/19) |
| 133 | Cross Sp | 19 | [4966-4984](19/19) | [5011-5029](19/19) |
| 134 | Cross Sp | 19 | [1733-1751](19/19) | [1778-1796](19/19) |
| 135 | Cross Sp | 19 | [4881-4899](19/19) | [4926-4944](19/19) |
| 136 | Cross Sp | 19 | [4879-4897](19/19) | [4924-4942](19/19) |
| 137 | Cross Sp | 19 | [4277-4295](19/19) | [4322-4340](19/19) |
| 138 | Cross Sp | 19 | [4267-4285](19/19) | [4312-4330](19/19) |
| 139 | Cross Sp | 19 | [4266-4284](19/19) | [4311-4329](19/19) |
| 140 | Cross Sp | 19 | [4265-4283](19/19) | [4310-4328](19/19) |
| 141 | Cross Sp | 19 | [4264-4282](19/19) | [4309-4327](19/19) |
| 142 | Cross Sp | 19 | [4010-4028](19/19) | [4055-4073](19/19) |
| 143 | Cross Sp | 19 | [3882-3900](19/19) | [3927-3945](19/19) |
| 144 | Cross Sp | 19 | [3881-3899](19/19) | [3926-3944](19/19) |
| 145 | Cross Sp | 19 | [3880-3898](19/19) | [3925-3943](19/19) |
| 146 | Cross Sp | 19 | [250-268](19/19) | [295-313](19/19) |
| 147 | Cross Sp | 19 | [3550-3568](19/19) | [3595-3613](19/19) |
| 148 | Cross Sp | 19 | [2662-2680](19/19) | [2707-2725](19/19) |
| 149 | Cross Sp | 19 | [2614-2632](19/19) | [2659-2677](19/19) |
| 150 | Cross Sp | 19 | [2382-2400](19/19) | [2427-2445](19/19) |
| 151 | Cross Sp | 19 | [2381-2399](19/19) | [2426-2444](19/19) |
| 152 | Cross Sp | 19 | [2146-2164](19/19) | [2191-2209](19/19) |
| 153 | Cross Sp | 19 | [2142-2160](19/19) | [2187-2205](19/19) |
| 154 | Cross Sp | 19 | [2140-2158](19/19) | [2185-2203](19/19) |
| 155 | Cross Sp | 19 | [1923-1941](19/19) | [1968-1986](19/19) |
| 156 | Cross Sp | 19 | [1919-1937](19/19) | [1964-1982](19/19) |
| 157 | Human | 19 | — | — |
| 158 | Human | 19 | [6004-6014](11/11) | [874-887](14/14) |
| 159 | Human | 19 | — | — |
| 160 | Human | 19 | — | — |
| 161 | Human | 19 | — | — |
| 162 | Human | 19 | — | — |
| 163 | Human | 19 | — | — |
| 164 | Human | 19 | — | — |
| 165 | Human | 19 | — | — |
| 166 | Human | 19 | — | — |
| 167 | Human | 19 | — | — |
| 168 | Human | 19 | — | — |
| 169 | Human | 19 | — | — |
| 170 | Human | 19 | — | — |
| 171 | Human | 19 | — | — |
| 172 | Human | 19 | [5300-5310](11/11) | — |
| 173 | Human | 19 | [4759-4774](16/16) | [4807-4819](13/13) |
| 174 | Human | 19 | [4746-4764](18/19) | [4791-4802](12/12) |
| 175 | Human | 19 | [4742-4757](16/16) | [4787-4802](16/16) |
| 176 | Human | 19 | [4892-4903](12/12) | [4937-4948](12/12) |
| 177 | Human | 19 | [4513-4526](14/14) | [4558-4571](14/14) |
| 178 | Human | 19 | [536-544](18/19) | [581-599](19/19) |
| 179 | Human | 19 | — | — |
| 180 | Human | 19 | [464-479](16/16) | [509-527](19/19) |
| 181 | Human | 19 | [3427-3443](17/17) | [3472-3488](16/17) |
| 182 | Human | 19 | — | — |
| 183 | Human | 19 | — | — |
| 184 | Human | 19 | [391-409](19/19) | [436-454](18/19) |
| 185 | Human | 19 | — | — |
| 186 | Human | 19 | — | — |
| 187 | Human | 19 | — | — |
| 188 | Human | 19 | — | — |
| 189 | Human | 19 | [1567-1577](11/11) | [1604-1622](18/19) |
| 190 | Human | 19 | — | — |
| 191 | Human | 19 | [226-243](17/18) | [270-288](19/19) |
| 192 | Human | 19 | — | — |
| 193 | Human | 19 | [1038-1049](12/12) | [1082-1094](13/13) |
| 194 | Human | 19 | [976-986](11/11) | [1021-1031](11/11) |
| 195 | Human | 19 | [969-986](17/18) | [1014-1031](17/18) |
| 196 | Human | 19 | [186-197](12/12) | [231-248](17/18) |
| 197 | Human | 19 | — | — |
| 198 | Human | 19 | — | — |
| 199 | Human | 19 | — | — |
| 200 | Human | 19 | — | — |
| 201 | Human | 19 | [916-934](18/19) | [961-979](18/19) |
| 202 | Human | 19 | — | [946-964](18/19) |
| 203 | Human | 19 | — | — |
| 204 | Human | 19 | — | — |
| 205 | Human | 19 | — | — |
| 206 | Human | 19 | — | — |
| 207 | Human | 19 | [1367-1377](11/11) | — |
| 208 | Human | 19 | [1367-1377](11/11) | — |
| 209 | Human | 19 | — | — |
| 210 | Human | 19 | — | — |
| 211 | Human | 19 | — | — |
| 212 | Human | 19 | — | — |
| 213 | Human | 19 | — | — |
| 214 | Human | 19 | — | — |
| 215 | Human | 19 | — | — |
| 216 | Human | 19 | — | — |
| 217 | Human | 19 | — | — |
| 218 | Human | 19 | — | — |
| 219 | Human | 19 | — | — |
| 220 | Human | 19 | — | — |
| 221 | Human | 19 | [2941-2951](11/11) | [2986-2996](11/11) |
| 222 | Human | 19 | — | — |
| 223 | Human | 19 | — | — |
| 224 | Human | 19 | — | — |
| 225 | Human | 19 | — | — |
| 226 | Human | 19 | — | — |
| 227 | Human | 19 | — | — |
| 228 | Human | 19 | — | — |
| 229 | Human | 19 | — | — |
| 230 | Human | 19 | — | — |
| 231 | Human | 19 | — | — |
| 232 | Human | 19 | — | — |
| 233 | Human | 19 | — | — |
| 234 | Human | 19 | — | — |
| 235 | Human | 19 | — | — |
| 236 | Human | 19 | [750-768](19/19) | [2184-2194](11/11) |
| 237 | Human | 19 | — | — |
| 238 | Human | 19 | — | — |
| 239 | Human | 19 | — | — |
| 240 | Human | 19 | — | — |
| 241 | Human | 19 | — | — |
| 242 | Human | 19 | — | — |
| 243 | Human | 19 | — | — |
| 244 | Human | 19 | — | — |
| 245 | Human | 19 | — | — |
| 246 | Human | 19 | — | — |
| 247 | Human | 19 | — | — |
| 248 | Human | 19 | — | — |
| 249 | Human | 19 | — | — |
| 250 | Human | 19 | — | — |
| 251 | Human | 19 | — | — |
| 252 | Human | 19 | — | — |
| 253 | Human | 19 | — | — |

FOR 21
STRETCHES:

| | | | | |
|---|---|---|---|---|
| 1 | Cross Sp | 21 | [1924-1944](21/21) | [1969-1989](21/21) |
| 2 | Cross Sp | 21 | [3876-3896](21/21) | [3921-3941](21/21) |
| 3 | Cross Sp | 21 | [637-657](21/21) | [682-702](21/21) |
| 4 | Cross Sp | 21 | [1897-1917](21/21) | [1942-1962](21/21) |
| 5 | Cross Sp | 21 | [5036-5056](21/21) | [5081-5101](21/21) |
| 6 | Cross Sp | 21 | [5026-5046](21/21) | [5071-5091](21/21) |
| 7 | Cross Sp | 21 | [4880-4900](21/21) | [4925-4945](21/21) |
| 8 | Cross Sp | 21 | [4297-4317](21/21) | [4342-4362](21/21) |
| 9 | Cross Sp | 21 | [1351-1371](21/21) | [1396-1416](21/21) |
| 10 | Cross Sp | 21 | [4008-4028](21/21) | [4053-4073](21/21) |
| 11 | Cross Sp | 21 | [3868-3888](21/21) | [3913-3933](21/21) |
| 12 | Cross Sp | 21 | [2658-2678](21/21) | [2703-2723](21/21) |
| 13 | Cross Sp | 21 | [2445-2465](21/21) | [2490-2510](21/21) |
| 14 | Cross Sp | 21 | [1926-1946](21/21) | [1971-1991](21/21) |
| 15 | Cross Sp | 21 | [1910-1930](21/21) | [1955-1975](21/21) |
| 16 | Cross Sp | 21 | [1894-1914](21/21) | [1939-1959](21/21) |
| 17 | Cross Sp | 21 | [1888-1908](21/21) | [1933-1953](21/21) |
| 18 | Cross Sp | 21 | [1883-1903](21/21) | [1928-1948](21/21) |
| 19 | Cross Sp | 21 | [5043-5063](21/21) | [5088-5108](21/21) |
| 20 | Cross Sp | 21 | [5040-5060](21/21) | [5085-5105](21/21) |
| 21 | Cross Sp | 21 | [5035-5055](21/21) | [5080-5100](21/21) |
| 22 | Cross Sp | 21 | [5032-5052](21/21) | [5077-5097](21/21) |
| 23 | Cross Sp | 21 | [5031-5051](21/21) | [5076-5096](21/21) |
| 24 | Cross Sp | 21 | [5028-5048](21/21) | [5073-5093](21/21) |
| 25 | Cross Sp | 21 | [4877-4897](21/21) | [4922-4942](21/21) |
| 26 | Cross Sp | 21 | [4459-4479](21/21) | [4504-4524](21/21) |
| 27 | Cross Sp | 21 | [4267-4287](21/21) | [4312-4332](21/21) |
| 28 | Cross Sp | 21 | [3879-3899](21/21) | [3924-3944](21/21) |
| 29 | Cross Sp | 21 | [3873-3893](21/21) | [3918-3938](21/21) |
| 30 | Cross Sp | 21 | [3547-3567](21/21) | [3592-3612](21/21) |
| 31 | Cross Sp | 21 | [3327-3347](21/21) | [3372-3392](21/21) |
| 32 | Cross Sp | 21 | [2664-2684](21/21) | [2709-2729](21/21) |
| 33 | Cross Sp | 21 | [2662-2682](21/21) | [2707-2727](21/21) |
| 34 | Cross Sp | 21 | [2656-2676](21/21) | [2701-2721](21/21) |
| 35 | Cross Sp | 21 | [2472-2492](21/21) | [2517-2537](21/21) |
| 36 | Cross Sp | 21 | [2448-2468](21/21) | [2493-2513](21/21) |
| 37 | Cross Sp | 21 | [2441-2461](21/21) | [2486-2506](21/21) |
| 38 | Cross Sp | 21 | [2439-2459](21/21) | [2484-2504](21/21) |
| 39 | Cross Sp | 21 | [2435-2455](21/21) | [2480-2500](21/21) |
| 40 | Cross Sp | 21 | [2433-2453](21/21) | [2478-2498](21/21) |
| 41 | Cross Sp | 21 | [2432-2452](21/21) | [2477-2497](21/21) |
| 42 | Cross Sp | 21 | [2431-2451](21/21) | [2476-2496](21/21) |
| 43 | Cross Sp | 21 | [2142-2162](21/21) | [2187-2207](21/21) |
| 44 | Cross Sp | 21 | [1932-1952](21/21) | [1977-1997](21/21) |
| 45 | Cross Sp | 21 | [1929-1949](21/21) | [1974-1994](21/21) |
| 46 | Cross Sp | 21 | [1920-1940](21/21) | [1965-1985](21/21) |
| 47 | Cross Sp | 21 | [458-478](21/21) | [503-523](21/21) |
| 48 | Cross Sp | 21 | [1908-1928](21/21) | [1953-1973](21/21) |
| 49 | Cross Sp | 21 | [1907-1927](21/21) | [1952-1972](21/21) |
| 50 | Cross Sp | 21 | [1904-1924](21/21) | [1949-1969](21/21) |
| 51 | Cross Sp | 21 | [1901-1921](21/21) | [1946-1966](21/21) |
| 52 | Cross Sp | 21 | [1893-1913](21/21) | [1938-1958](21/21) |
| 53 | Cross Sp | 21 | [1891-1911](21/21) | [1936-1956](21/21) |
| 54 | Cross Sp | 21 | [1890-1910](21/21) | [1935-1955](21/21) |
| 55 | Cross Sp | 21 | [1887-1907](21/21) | [1932-1952](21/21) |
| 56 | Cross Sp | 21 | [1886-1906](21/21) | [1931-1951](21/21) |
| 57 | Cross Sp | 21 | [1884-1904](21/21) | [1929-1949](21/21) |
| 58 | Cross Sp | 21 | [1882-1902](21/21) | [1927-1947](21/21) |
| 59 | Cross Sp | 21 | [5041-5061](21/21) | [5086-5106](21/21) |
| 60 | Cross Sp | 21 | [5038-5058](21/21) | [5083-5103](21/21) |
| 61 | Cross Sp | 21 | [5037-5057](21/21) | [5082-5102](21/21) |
| 62 | Cross Sp | 21 | [5034-5054](21/21) | [5079-5099](21/21) |
| 63 | Cross Sp | 21 | [5033-5053](21/21) | [5078-5098](21/21) |
| 64 | Cross Sp | 21 | [5029-5049](21/21) | [5074-5094](21/21) |
| 65 | Cross Sp | 21 | [5023-5043](21/21) | [5068-5088](21/21) |
| 66 | Cross Sp | 21 | [4879-4899](21/21) | [4924-4944](21/21) |
| 67 | Cross Sp | 21 | [4461-4481](21/21) | [4506-4526](21/21) |
| 68 | Cross Sp | 21 | [4299-4319](21/21) | [4344-4364](21/21) |
| 69 | Cross Sp | 21 | [4271-4291](21/21) | [4316-4336](21/21) |
| 70 | Cross Sp | 21 | [4265-4285](21/21) | [4310-4330](21/21) |
| 71 | Cross Sp | 21 | [4264-4284](21/21) | [4309-4329](21/21) |
| 72 | Cross Sp | 21 | [4262-4282](21/21) | [4307-4327](21/21) |
| 73 | Cross Sp | 21 | [4248-4268](21/21) | [4293-4313](21/21) |
| 74 | Cross Sp | 21 | [4247-4267](21/21) | [4292-4312](21/21) |
| 75 | Cross Sp | 21 | [3880-3900](21/21) | [3925-3945](21/21) |
| 76 | Cross Sp | 21 | [3878-3898](21/21) | [3923-3943](21/21) |
| 77 | Cross Sp | 21 | [3872-3892](21/21) | [3917-3937](21/21) |
| 78 | Cross Sp | 21 | [3552-3572](21/21) | [3597-3617](21/21) |
| 79 | Cross Sp | 21 | [3550-3570](21/21) | [3595-3615](21/21) |
| 80 | Cross Sp | 21 | [3548-3568](21/21) | [3593-3613](21/21) |
| 81 | Cross Sp | 21 | [3389-3409](21/21) | [3434-3454](21/21) |
| 82 | Cross Sp | 21 | [3326-3346](21/21) | [3371-3391](21/21) |
| 83 | Cross Sp | 21 | [3325-3345](21/21) | [3370-3390](21/21) |
| 84 | Cross Sp | 21 | [2661-2681](21/21) | [2706-2726](21/21) |
| 85 | Cross Sp | 21 | [2660-2680](21/21) | [2705-2725](21/21) |
| 86 | Cross Sp | 21 | [2659-2679](21/21) | [2704-2724](21/21) |
| 87 | Cross Sp | 21 | [2612-2632](21/21) | [2657-2677](21/21) |
| 88 | Cross Sp | 21 | [2611-2631](21/21) | [2656-2676](21/21) |
| 89 | Cross Sp | 21 | [2471-2491](21/21) | [2516-2536](21/21) |
| 90 | Cross Sp | 21 | [2470-2490](21/21) | [2515-2535](21/21) |
| 91 | Cross Sp | 21 | [2443-2463](21/21) | [2488-2508](21/21) |
| 92 | Cross Sp | 21 | [2438-2458](21/21) | [2483-2503](21/21) |
| 93 | Cross Sp | 21 | [2437-2457](21/21) | [2482-2502](21/21) |
| 94 | Cross Sp | 21 | [2436-2456](21/21) | [2481-2501](21/21) |
| 95 | Cross Sp | 21 | [2434-2454](21/21) | [2479-2499](21/21) |
| 96 | Cross Sp | 21 | [2380-2400](21/21) | [2425-2445](21/21) |
| 97 | Cross Sp | 21 | [2144-2164](21/21) | [2189-2209](21/21) |
| 98 | Cross Sp | 21 | [2143-2163](21/21) | [2188-2208](21/21) |
| 99 | Cross Sp | 21 | [1930-1950](21/21) | [1975-1995](21/21) |
| 100 | Cross Sp | 21 | [1928-1948](21/21) | [1973-1993](21/21) |
| 101 | Cross Sp | 21 | [4275-4295](21/21) | [4320-4340](21/21) |
| 102 | Cross Sp | 21 | [4273-4293](21/21) | [4318-4338](21/21) |
| 103 | Cross Sp | 21 | [4268-4288](21/21) | [4313-4333](21/21) |
| 104 | Cross Sp | 21 | [4007-4027](21/21) | [4052-4072](21/21) |
| 105 | Cross Sp | 21 | [1903-1923](21/21) | [1948-1968](21/21) |
| 106 | Cross Sp | 21 | [4006-4026](21/21) | [4051-4071](21/21) |
| 107 | Cross Sp | 21 | [4005-4025](21/21) | [4050-4070](21/21) |
| 108 | Cross Sp | 21 | [4004-4024](21/21) | [4049-4069](21/21) |
| 109 | Cross Sp | 21 | [4003-4023](21/21) | [4048-4068](21/21) |
| 110 | Cross Sp | 21 | [3875-3895](21/21) | [3920-3940](21/21) |
| 111 | Cross Sp | 21 | [3874-3894](21/21) | [3919-3939](21/21) |
| 112 | Cross Sp | 21 | [3870-3890](21/21) | [3915-3935](21/21) |
| 113 | Cross Sp | 21 | [3549-3569](21/21) | [3594-3614](21/21) |
| 114 | Cross Sp | 21 | [2657-2677](21/21) | [2702-2722](21/21) |
| 115 | Cross Sp | 21 | [2613-2633](21/21) | [2658-2678](21/21) |
| 116 | Cross Sp | 21 | [2610-2630](21/21) | [2655-2675](21/21) |
| 117 | Cross Sp | 21 | [1732-1752](21/21) | [1777-1797](21/21) |
| 118 | Cross Sp | 21 | [1925-1945](21/21) | [1970-1990](21/21) |
| 119 | Cross Sp | 21 | [1923-1943](21/21) | [1968-1988](21/21) |
| 120 | Cross Sp | 21 | [1922-1942](21/21) | [1967-1987](21/21) |
| 121 | Cross Sp | 21 | [1917-1937](21/21) | [1962-1982](21/21) |
| 122 | Cross Sp | 21 | [1916-1936](21/21) | [1961-1981](21/21) |
| 123 | Cross Sp | 21 | [1913-1933](21/21) | [1958-1978](21/21) |
| 124 | Cross Sp | 21 | [1911-1931](21/21) | [1956-1976](21/21) |
| 125 | Cross Sp | 21 | [1909-1929](21/21) | [1954-1974](21/21) |
| 126 | Cross Sp | 21 | [1906-1926](21/21) | [1951-1971](21/21) |
| 127 | Cross Sp | 21 | [4965-4985](21/21) | [5010-5030](21/21) |
| 128 | Cross Sp | 21 | [4964-4984](21/21) | [5009-5029](21/21) |
| 129 | Cross Sp | 21 | [1905-1925](21/21) | [1950-1970](21/21) |
| 130 | Cross Sp | 21 | [4270-4290](21/21) | [4315-4335](21/21) |
| 131 | Cross Sp | 21 | [4266-4286](21/21) | [4311-4331](21/21) |
| 132 | Cross Sp | 21 | [4009-4029](21/21) | [4054-4074](21/21) |
| 133 | Cross Sp | 21 | [3877-3897](21/21) | [3922-3942](21/21) |
| 134 | Cross Sp | 21 | [3871-3891](21/21) | [3916-3936](21/21) |
| 135 | Cross Sp | 21 | [3869-3889](21/21) | [3914-3934](21/21) |
| 136 | Cross Sp | 21 | [1733-1753](21/21) | [1778-1798](21/21) |
| 137 | Cross Sp | 21 | [2140-2160](21/21) | [2185-2205](21/21) |
| 138 | Cross Sp | 21 | [1914-1934](21/21) | [1959-1979](21/21) |
| 139 | Cross Sp | 21 | [1912-1932](21/21) | [1957-1977](21/21) |
| 140 | Cross Sp | 21 | [4881-4901](21/21) | [4926-4946](21/21) |
| 141 | Cross Sp | 21 | [459-479](21/21) | [504-524](21/21) |
| 142 | Cross Sp | 21 | [4274-4294](21/21) | [4319-4339](21/21) |
| 143 | Cross Sp | 21 | [4272-4292](21/21) | [4317-4337](21/21) |
| 144 | Cross Sp | 21 | [1734-1754](21/21) | [1779-1799](21/21) |
| 145 | Cross Sp | 21 | [2609-2629](21/21) | [2654-2674](21/21) |
| 146 | Cross Sp | 21 | [2141-2161](21/21) | [2186-2206](21/21) |
| 147 | Cross Sp | 21 | [1919-1939](21/21) | [1964-1984](21/21) |
| 148 | Cross Sp | 21 | [1915-1935](21/21) | [1960-1980](21/21) |
| 149 | Cross Sp | 21 | [4878-4898](21/21) | [4923-4943](21/21) |
| 150 | Cross Sp | 21 | [4269-4289](21/21) | [4314-4334](21/21) |
| 151 | Cross Sp | 21 | [4010-4030](21/21) | [4055-4075](21/21) |
| 152 | Cross Sp | 21 | [2148-2168](21/21) | [2193-2213](21/21) |
| 153 | Cross Sp | 21 | [4276-4296](21/21) | [4321-4341](21/21) |
| 154 | Cross Sp | 21 | [1921-1941](21/21) | [1966-1986](21/21) |
| 155 | Cross Sp | 21 | [1918-1938](21/21) | [1963-1983](21/21) |
| 156 | Cross Sp | 21 | [4279-4299](21/21) | [4324-4344](21/21) |
| 157 | Cross Sp | 21 | [4011-4031](21/21) | [4056-4076](21/21) |
| 158 | Cross Sp | 21 | [2146-2166](21/21) | [2191-2211](21/21) |
| 159 | Cross Sp | 21 | [2145-2165](21/21) | [2190-2210](21/21) |
| 160 | Cross Sp | 21 | [4277-4297](21/21) | [4322-4342](21/21) |
| 161 | Cross Sp | 21 | [4280-4300](21/21) | [4325-4345](21/21) |
| 162 | Human | 21 | — | [1082-1092](11/11) |
| 163 | Human | 21 | — | — |
| 164 | Human | 21 | — | [945-965](20/21) |
| 165 | Human | 21 | — | — |
| 166 | Human | 21 | — | — |
| 167 | Human | 21 | — | — |
| 168 | Human | 21 | — | — |
| 169 | Human | 21 | — | — |
| 170 | Human | 21 | — | — |
| 171 | Human | 21 | — | — |
| 172 | Human | 21 | — | — |
| 173 | Human | 21 | [616-636](20/21) | [661-680](20/20) |
| 174 | Human | 21 | — | — |
| 175 | Human | 21 | — | — |
| 176 | Human | 21 | [4984-5004](20/21) | — |
| 177 | Human | 21 | [4924-4943](19/20) | [4972-4988](16/17) |
| 178 | Human | 21 | [4892-4903](12/12) | [4937-4948](12/12) |
| 179 | Human | 21 | — | — |
| 180 | Human | 21 | — | — |
| 181 | Human | 21 | [4394-4414](20/21) | [4439-4459](20/21) |
| 182 | Human | 21 | [528-548](20/21) | [573-593](21/21) |
| 183 | Human | 21 | [526-542](17/17) | [571-588](18/18) |
| 184 | Human | 21 | [4240-4256](16/17) | [4281-4301](19/21) |
| 185 | Human | 21 | — | [4027-4041](15/15) |
| 186 | Human | 21 | [3916-3936](20/21) | — |
| 187 | Human | 21 | — | — |
| 188 | Human | 21 | [3427-3444](18/18) | [3472-3489](17/18) |
| 189 | Human | 21 | [3339-3359](20/21) | [3384-3404](20/21) |
| 190 | Human | 21 | [3223-3242](18/20) | [3268-3287](18/20) |
| 191 | Human | 21 | [3085-3104](19/20) | [3129-3149](21/21) |
| 192 | Human | 21 | [3085-3098](14/14) | [3127-3146](20/20) |
| 193 | Human | 21 | — | — |
| 194 | Human | 21 | [2883-2903](20/21) | [2928-2948](20/21) |
| 195 | Human | 21 | [2782-2798](16/17) | [2827-2843](16/17) |
| 196 | Human | 21 | [345-365](21/21) | [390-410](20/21) |
| 197 | Human | 21 | [2388-2408](20/21) | [2433-2453](20/21) |
| 198 | Human | 21 | — | — |
| 199 | Human | 21 | — | [1666-1686](21/21) |
| 200 | Human | 21 | — | — |
| 201 | Human | 21 | [3591-3602](12/12) | — |
| 202 | Human | 21 | — | — |
| 203 | Human | 21 | — | — |
| 204 | Human | 21 | [181-197](17/17) | [226-242](17/17) |
| 205 | Human | 21 | — | [943-963](20/21) |
| 206 | Human | 21 | [895-912](17/18) | [940-957](17/18) |
| 207 | Human | 21 | — | — |
| 208 | Human | 21 | [6004-6014](11/11) | [874-887](14/14) |
| 209 | Human | 21 | [1367-1377](11/11) | — |
| 210 | Human | 21 | — | — |
| 211 | Human | 21 | — | — |
| 212 | Human | 21 | [1367-1377](11/11) | — |
| 213 | Human | 21 | — | — |
| 214 | Human | 21 | — | — |
| 215 | Human | 21 | — | — |
| 216 | Human | 21 | — | — |
| 217 | Human | 21 | — | — |
| 218 | Human | 21 | — | — |
| 219 | Human | 21 | — | — |
| 220 | Human | 21 | — | — |
| 221 | Human | 21 | — | — |
| 222 | Human | 21 | — | [820-830](11/11) |
| 223 | Human | 21 | — | — |
| 224 | Human | 21 | — | — |
| 225 | Human | 21 | — | — |
| 226 | Human | 21 | — | — |
| 227 | Human | 21 | — | — |
| 228 | Human | 21 | — | — |
| 229 | Human | 21 | — | — |
| 230 | Human | 21 | — | — |
| 231 | Human | 21 | — | — |
| 232 | Human | 21 | — | — |
| 233 | Human | 21 | — | — |
| 234 | Human | 21 | [749-763](15/15) | — |
| 235 | Human | 21 | — | — |
| 236 | Human | 21 | — | — |
| 237 | Human | 21 | — | — |
| 238 | Human | 21 | — | — |
| 239 | Human | 21 | — | — |
| 240 | Human | 21 | — | — |
| 241 | Human | 21 | — | — |
| 242 | Human | 21 | — | — |
| 243 | Human | 21 | — | — |
| 244 | Human | 21 | — | — |
| 245 | Human | 21 | — | — |
| 246 | Human | 21 | — | — |
| 247 | Human | 21 | — | — |
| 248 | Human | 21 | — | — |
| 249 | Human | 21 | — | — |
| 250 | Human | 21 | — | — |
| 251 | Human | 21 | — | — |
| 252 | Human | 21 | — | — |
| 253 | Human | 21 | — | — |
| 254 | Human | 21 | — | — |
| 255 | Human | 21 | — | — |
| 256 | Human | 21 | [681-701](20/21) | [726-746](20/21) |
| 257 | Human | 21 | — | — |
| 258 | Human | 21 | [679-695](17/17) | [724-740](17/17) |
| 259 | Human | 21 | — | — |

Table G below shows further siRNA compounds specific for the ODC1 gene that have been selected according to the present invention

TABLE G

| | | | | | |
|---|---|---|---|---|---|
| | | Oligo | | | |
| No | Source | Length | Sense siRNA | AntiSense siRNA | human 4505488 |

FOR 19
STRETCHES:

| | | | | | |
|---|---|---|---|---|---|
| 1 | Cross Sp | 19 | GCAUGUGGGUGAUUGGAUG | CAUCCAAUCACCCACAUGC | [1459-1477](19/19) |
| 2 | Cross Sp | 19 | GAGUCCAGAUGAUGACUUU | AAAGUCAUCAUCUGGACUC | [714-732](19/19) |
| 3 | Cross Sp | 19 | GGAGUCCAGAUGAUGACUU | AAGUCAUCAUCUGGACUCC | [713-731](19/19) |
| 4 | Cross Sp | 19 | GCAAGACUGAAAUACAGUU | AACUGUAUUUCAGUCUUGC | [606-624](19/19) |
| 5 | Cross Sp | 19 | CUGAAAUACAGUUGGUGCA | UGCACCAACUGUAUUUCAG | [612-630](19/19) |
| 6 | Cross Sp | 19 | GAGUGAGAAUCAUAGCUGA | UCAGCUAUGAUUCUCACUC | [1137-1155](19/19) |
| 7 | Cross Sp | 19 | GGAGUGAGAAUCAUAGCUG | CAGCUAUGAUUCUCACUCC | [1136-1154](19/19) |
| 8 | Cross Sp | 19 | CCACUGAUGAUUCCAAAGC | GCUUUGGAAUCAUCAGUGG | [801-819](19/19) |
| 9 | Cross Sp | 19 | UGCCACUGAUGAUUCCAAA | UUUGGAAUCAUCAGUGGCA | [799-817](19/19) |
| 10 | Cross Sp | 19 | CAAGACUGAAAUACAGUUG | CAACUGUAUUUCAGUCUUG | [607-625](19/19) |
| 11 | Cross Sp | 19 | CGGAUUGCCACUGAUGAUU | AAUCAUCAGUGGCAAUCCG | [794-812](19/19) |
| 12 | Cross Sp | 19 | UCCAGAUGAUGACUUUUGA | UCAAAAGUCAUCAUCUGGA | [717-735](19/19) |
| 13 | Cross Sp | 19 | GACUGAAAUACAGUUGGUG | CACCAACUGUAUUUCAGUC | [610-628](19/19) |
| 14 | Cross Sp | 19 | UGAAAUGCAUGUGGGUGAU | AUCACCCACAUGCAUUUCA | [1453-1471](19/19) |
| 15 | Cross Sp | 19 | CUGAAAUGCAUGUGGGUGA | UCACCCACAUGCAUUUCAG | [1452-1470](19/19) |
| 16 | Cross Sp | 19 | GCCUGAAAUGCAUGUGGGU | ACCCACAUGCAUUUCAGGC | [1450-1468](19/19) |
| 17 | Cross Sp | 19 | CUGCCUGAAAUGCAUGUGG | CCACAUGCAUUUCAGGCAG | [1448-1466](19/19) |
| 18 | Cross Sp | 19 | AGACUGAAAUACAGUUGGU | ACCAACUGUAUUUCAGUCU | [609-627](19/19) |
| 19 | Cross Sp | 19 | UGGGGACCAACAUGUGAUG | CAUCACAUGUUGGUCCCCA | [1400-1418](19/19) |
| 20 | Cross Sp | 19 | UGGAGUGAGAAUCAUAGCU | AGCUAUGAUUCUCACUCCA | [1135-1153](19/19) |
| 21 | Cross Sp | 19 | UCUGGAGUGAGAAUCAUAG | CUAUGAUUCUCACUCCAGA | [1133-1151](19/19) |
| 22 | Cross Sp | 19 | CUCUGGAGUGAGAAUCAUA | UAUGAUUCUCACUCCAGAG | [1132-1150](19/19) |
| 23 | Cross Sp | 19 | ACUCUGGAGUGAGAAUCAU | AUGAUUCUCACUCCAGAGU | [1131-1149](19/19) |
| 24 | Cross Sp | 19 | GACUCUGGAGUGAGAAUCA | UGAUUCUCACUCCAGAGUC | [1130-1148](19/19) |
| 25 | Cross Sp | 19 | UUGCCACUGAUGAUUCCAA | UUGGAAUCAUCAGUGGCAA | [798-816](19/19) |
| 26 | Cross Sp | 19 | GAUUGCCACUGAUGAUUCC | GGAAUCAUCAGUGGCAAUC | [796-814](19/19) |
| 27 | Cross Sp | 19 | GGAUUGCCACUGAUGAUUC | GAAUCAUCAGUGGCAAUCC | [795-813](19/19) |
| 28 | Cross Sp | 19 | GUCCAGAUGAUGACUUUUG | CAAAAGUCAUCAUCUGGAC | [716-734](19/19) |
| 29 | Cross Sp | 19 | UCUAUGCAAAUCCUUGUAA | UUACAAGGAUUUGCAUAGA | [660-678](19/19) |
| 30 | Cross Sp | 19 | AUCUAUGCAAAUCCUUGUA | UACAAGGAUUUGCAUAGAU | [659-677](19/19) |
| 31 | Cross Sp | 19 | UGCAUGUGGGUGAUUGGAU | AUCCAAUCACCCACAUGCA | [1458-1476](19/19) |
| 32 | Cross Sp | 19 | AUGCAUGUGGGUGAUUGGA | UCCAAUCACCCACAUGCAU | [1457-1475](19/19) |
| 33 | Cross Sp | 19 | AAUGCAUGUGGGUGAUUGG | CCAAUCACCCACAUGCAUU | [1456-1474](19/19) |
| 34 | Cross Sp | 19 | AAAUGCAUGUGGGUGAUUG | CAAUCACCCACAUGCAUUU | [1455-1473](19/19) |
| 35 | Cross Sp | 19 | GAAAUGCAUGUGGGUGAUU | AAUCACCCACAUGCAUUUC | [1454-1472](19/19) |
| 36 | Cross Sp | 19 | UGCCUGAAAUGCAUGUGGG | CCCACAUGCAUUUCAGGCA | [1449-1467](19/19) |
| 37 | Cross Sp | 19 | CCUGCCUGAAAUGCAUGUG | CACAUGCAUUUCAGGCAGG | [1447-1465](19/19) |
| 38 | Cross Sp | 19 | GACCAACAUGUGAUGGCCU | AGGCCAUCACAUGUUGUC | [1404-1422](19/19) |
| 39 | Cross Sp | 19 | UGAGAAUCAUAGCUGAGCC | GGCUCAGCUAUGAUUCUCA | [1140-1158](19/19) |
| 40 | Cross Sp | 19 | CUGGAGUGAGAAUCAUAGC | GCUAUGAUUCUCACUCCAG | [1134-1152](19/19) |
| 41 | Cross Sp | 19 | AAGACUGAAAUACAGUUGG | CCAACUGUAUUUCAGUCUU | [608-626](19/19) |
| 42 | Cross Sp | 19 | GCCACUGAUGAUUCCAAAG | CUUUGGAAUCAUCAGUGGC | [800-818](19/19) |
| 43 | Cross Sp | 19 | UAUCUAUGCAAAUCCUUGU | ACAAGGAUUUGCAUAGAUA | [658-676](19/19) |
| 44 | Cross Sp | 19 | AGCAAGACUGAAAUACAGU | ACUGUAUUUCAGUCUUGCU | [605-623](19/19) |
| 45 | Cross Sp | 19 | UUAUCUAUGCAAAUCCUUG | CAAGGAUUUGCAUAGAUAA | [657-675](19/19) |
| 46 | Cross Sp | 19 | AUGUGGGUGAUUGGAUGCU | AGCAUCCAAUCACCCACAU | [1461-1479](19/19) |
| 47 | Cross Sp | 19 | CAUGUGGGUGAUUGGAUGC | GCAUCCAAUCACCCACAUG | [1460-1478](19/19) |
| 48 | Cross Sp | 19 | GUGAGAAUCAUAGCUGAGC | GCUCAGCUAUGAUUCUCAC | [1139-1157](19/19) |
| 49 | Cross Sp | 19 | AUUGCCACUGAUGAUUCCA | UGGAAUCAUCAGUGGCAAU | [797-815](19/19) |
| 50 | Cross Sp | 19 | AGUCCAGAUGAUGACUUUU | AAAAGUCAUCAUCUGGACU | [715-733](19/19) |
| 51 | Cross Sp | 19 | UGAAAUACAGUUGGUGCAG | CUGCACCAACUGUAUUUCA | [613-631](19/19) |
| 52 | Cross Sp | 19 | ACUGAAAUACAGUUGGUGC | GCACCAACUGUAUUUCAGU | [611-629](19/19) |
| 53 | Cross Sp | 19 | CCUGAAAUGCAUGUGGGUG | CACCCACAUGCAUUUCAGG | [1451-1469](19/19) |
| 54 | Cross Sp | 19 | AGUGAGAAUCAUAGCUGAG | CUCAGCUAUGAUUCUCACU | [1138-1156](19/19) |
| 55 | Cross Sp | 19 | GGACCAACAUGUGAUGGCC | GGCCAUCACAUGUUUGUCC | [1403-1421](19/19) |
| 56 | Cross Sp | 19 | GGGACCAACAUGUGAUGGC | GCCAUCACAUGUUGGUCCC | [1402-1420](19/19) |
| 57 | Cross Sp | 19 | GGGGACCAACAUGUGAUGG | CCAUCACAUGUUGGUCCCC | [1401-1419](19/19) |
| 58 | Human | 19 | CGAGUGAGCACACCUUUAU | AUAAAGGUCUGCUCACUCG | [1260-1278](19/19) |
| 59 | Human | 19 | GCAUGUAUCUGCUUGAUAU | AUAUCAAGCAGAUACAUGC | [1017-1035](19/19) |
| 60 | Human | 19 | CCAAAGCAAAGUUGGUUUU | AAAACCAACUUUGCUUUGG | [774-792](19/19) |
| 61 | Human | 19 | GAACAACUUUGGUAAUGAA | UUCAUUACCAAAGUUGUUC | [337-355](19/19) |
| 62 | Human | 19 | GCAGUCAAAUGUAAUGAUA | UAUCAUUACAUUUGACUGC | [533-551](19/19) |
| 63 | Human | 19 | GGAUAUGGGUCACACUUAU | AUAAGUGUGACCCAUAUCC | [1858-1876](19/19) |
| 64 | Human | 19 | CCAUAUGGAAGACUAGGAU | AUCCUAGUCUUCCAUAUGG | [1843-1861](19/19) |
| 65 | Human | 19 | GCAGCCAUAUGGAAGACUA | UAGUCUUCCAUAUGGCUGC | [1839-1857](19/19) |
| 66 | Human | 19 | CGACGAUCUACUAUGUGAU | AUCACAUAGUAGAUCGUCG | [1542-1560](19/19) |
| 67 | Human | 19 | GCAAAAGAGACCUAAACCA | UGGUUUAGGUCUCUUUUGC | [1354-1372](19/19) |
| 68 | Human | 19 | GCUCUGAUGACGAAGAUGA | UCAUCUUCGUCAUCAGAGC | [1239-1257](19/19 |
| 69 | Human | 19 | GGGCUCUGAUGACGAAGAU | AUCUUCGUCAUCAGAGCCC | [1237-1255](19/19) |
| 70 | Human | 19 | GAAGUUUCUUCUUCUGAUG | CAUCAGAAGAAGAAACUUC | [422-440](19/19) |
| 71 | Human | 19 | UCAUUGCCAAGAAAAUUGU | ACAAUUUUCUUGGCAAUGA | [1203-1221](19/19) |
| 72 | Human | 19 | CAGCGUUGGACAAAUACUU | AAGUAUUUGUCCAACGCUG | [1104-1122](19/19) |
| 73 | Human | 19 | CUGAGGAUGUGAAACUUAA | UUAAGUUUCACAUCCUCAG | [1056-1074](19/19) |
| 74 | Human | 19 | CUGGAUCUGAGGAUGUGAA | UUCACAUCCUCAGAUCCAG | [1050-1068](19/19) |
| 75 | Human | 19 | GGACCAGAAAAUUAAUGAA | UUCAUUAAUUUUCUGGUCC | [406-424](19/19) |
| 76 | Human | 19 | ACAUUCUGGACCAGAAAAU | AUUUUCUGGUCCAGAAUGU | [399-417](19/19) |
| 77 | Human | 19 | GGACAUUCUGGACCAGAAA | UUUCUGGUCCAGAAUGUCC | [397-415](19/19) |
| 78 | Human | 19 | CGAAAGAGCUAAAUAUCGA | UCGAUAUUUAGCUCUUUCG | [885-903](19/19) |
| 79 | Human | 19 | GGGCGAAAGAGCUAAAUAU | AUAUUUAGCUCUUUCGCCC | [882-900](19/19) |
| 80 | Human | 19 | GAACCAGCAGGCUCCUUUU | AAAAGGAGCCUGCUGGUUC | [858-876](19/19) |
| 81 | Human | 19 | AGAACCAGCAGGCUCCUUU | AAAGGAGCCUGCUGGUUCU | [857-875](19/19) |
| 82 | Human | 19 | UUACUGCCAAGGACAUUCU | AGAAUGUCCUUGGCAGUAA | [387-405](19/19) |
| 83 | Human | 19 | CAGAGCACAUCCCAAAGCA | UGCUUUGGGAUGUGCUCUG | [763-781](19/19) |
| 84 | Human | 19 | GUGAAGUUGAGUUGAUGAA | UUCAUCAACUCAACUUCAC | [738-756](19/19) |
| 85 | Human | 19 | GAUGACUUUUGAUAGUGAA | UUCACUAUCAAAAGUCAUC | [724-742](19/19) |
| 86 | Human | 19 | CAGAUGAUGACUUUUGAUA | UAUCAAAAGUCAUCAUCUG | [719-737](19/19) |
| 87 | Human | 19 | CCUGGAUGAAGGUUUUACU | AGUAAAACCUUCAUCGAGG | [373-391](19/19) |
| 88 | Human | 19 | CUUCCUCGAUGAAGGUUUU | AAAACCUUCAUCGAGGAAG | [370-388](19/19) |
| 89 | Human | 19 | GCAAAUCCUUGUAAACAAG | CUUGUUUACAAGGAUUUGC | [665-683](19/19) |
| 90 | Human | 19 | CUAUGCAAAUCCUUGUAAA | UUUACAAGGAUUUGCAUAG | [661-679](19/19) |
| 91 | Human | 19 | AGAGGAUUAUCUAUGCAAA | UUUGCAUAGAUAAUCCUCU | [651-669](19/19) |
| 92 | Human | 19 | CCUCCAGAGAGGAUUAUCU | AGAUAAUCCUCUCUGGAGG | [644-662](19/19) |
| 93 | Human | 19 | GUGCUAGCAAGACUGAAAU | AUUUCAGUCUUGCUAGCAC | [600-618](19/19) |
| 94 | Human | 19 | GCAAAGCCAUCGUGAAGAC | GUCUUCACGAUGGCUUUGC | [552-570](19/19) |
| 95 | Human | 19 | CCCCUUUUAUGCAGUCAAA | UUUGACUGCAUAAAAGGGG | [523-541](19/19) |
| 96 | Human | 19 | GUUGUGACCUGUUUUUAAA | UUUAAAAACAGGUCACAAC | [2019-2037](19/19) |
| 97 | Human | 19 | UUAGUGUUGUGACCUGUUU | AAACAGGUCACAACACUAA | [2014-2032](19/19) |
| 98 | Human | 19 | GCAGAAUGGGCCAAAAGCU | AGCUUUUGGCCCAUUCUGC | [1996-2014](19/19) |
| 99 | Human | 19 | GCAUUUGUAGCUUGUACAA | UUGUACAAGCUACAAAUGC | [1975-1993](19/19) |
| 100 | Human | 19 | AGACACGCUACUCAAGAGU | ACUCUUGAGUAGCGUGUCU | [1936-1954](19/19) |
| 101 | Human | 19 | CCUAUGGAAACUAUUUGAA | UUCAAAUAGUUUCCAUAGG | [1884-1902](19/19) |
| 102 | Human | 19 | GUGUUCCUAUGGAAACUAU | AUAGUUUCCAUAGGAACAC | [1879-1897](19/19) |
| 103 | Human | 19 | AAACAUCUGAGGUGGUUAA | UUAACCACCUCAGAUGUUU | [485-503](19/19) |
| 104 | Human | 19 | CCAUGAUGCAGCCAUAUGG | CCAUAUGGCUGCAUCAUGG | [1832-1850](19/19) |
| 105 | Human | 19 | AGAGUAGGGUCGCCAUGAU | AUCAUGGCGACCCUACUCU | [1820-1838](19/19) |
| 106 | Human | 19 | GGACCAUGUAACUUAAUUA | UAAUUAAGUUACAUGGUCC | [1776-1794](19/19) |
| 107 | Human | 19 | GGGGGACCAUGUAACUUAA | UUAAGUUACAUGGUCCCCC | [1773-1791](19/19) |
| 108 | Human | 19 | GCAAGUUUAGCUUGAAUUA | UAAUUCAAGCUAAACUUGC | [1745-1763](19/19) |
| 109 | Human | 19 | GCUGUUAACUGCAAGUUUA | UAAACUUGCAGUUAACAGC | [1735-1753](19/19) |
| 110 | Human | 19 | GCACUCUGGUAGCUGUUAA | UUAACAGCUACCAGAGUGC | [1724-1742](19/19) |
| 111 | Human | 19 | GGGAGACAUUCUAAAGAAA | UUUCUUUAGAAUGUCUCCC | [469-487](19/19) |
| 112 | Human | 19 | AGACCUGGGAGACAUUCUA | UAGAAUGUCUCCCAGGUCU | [463-481](19/19) |
| 113 | Human | 19 | GGCAACUCAUGCAGCAAUU | AAUUGCUGCAUGAGUUGCC | [1575-1593](19/19) |
| 114 | Human | 19 | GGCCGACGAUCUACUAUGU | ACAUAGUAGAUCGUCGGCC | [1539-1557](19/19) |
| 115 | Human | 19 | GGAUGCUCUUUGAAAACAU | AUGUUUUCAAAGAGCAUCC | [1473-1491](19/19) |
| 116 | Human | 19 | GUGAUUGGAUGCUCUUUGA | UCAAAGAGCAUCCAAUCAC | [1467-1485](19/19) |
| 117 | Human | 19 | UGACCUGCCUGAAAUGCAU | AUGCAUUUCAGGCAGGUCA | [1444-1462](19/19) |
| 118 | Human | 19 | GCUGUGACCUGCCUGAAAU | AUUUCAGGCAGGUCACAGC | [1440-1458](19/19) |
| 119 | Human | 19 | UGAUAAGGAUGCCUUCUAU | AUAGAAGGCAUCCUUAUCA | [439-457](19/19) |
| 120 | Human | 19 | CCAGAUGAGAAGUAUUAUU | AAUAAUACUUCUCAUCUGG | [1370-1388](19/19) |
| 121 | Human | 19 | UGACCACGCACAUGGAAAG | CUUUACAUGUGCGUGUUCA | [1327-1345](19/19) |
| 122 | Human | 19 | CUAUGACCACGCACAUGUA | UACAUGUGCGUGGUCAUAG | [1324-1342](19/19) |
| 123 | Human | 19 | UCUAUGACCACGCACAUGU | ACAUGUGCGUGGUCAUAGA | [1323-1341](19/19) |
| 124 | Human | 19 | GGAUCAUUUAAUUGCAUAC | GUAUGCAAUUAAAUGAUCC | [1304-1322](19/19) |
| 125 | Human | 19 | CGUCUAUGGAUCAUUUAAU | AUUAAAUGAUCCAUAGACG | [1297-1315](19/19) |
| 126 | Human | 19 | GGCGUCUAUGGAUCAUUUA | UAAAUGAUCCAUAGACGCC | [1295-1313](19/19) |
| 127 | Human | 19 | UGGCGUCUAUGGAUCAUUU | AAAUGAUCCAUAGACGCCA | [1294-1312](19/19) |
| 128 | Human | 19 | GAAUGAUGGCGUCUAUGGA | UCCAUAGACGCCAUCAUUC | [1288-1306](19/19) |
| 129 | Human | 19 | UGUGAAUGAUGGCGUCUAU | AUAGACGCCAUCAUUCACA | [1285-1303](19/19) |
| 130 | Human | 19 | UUCUUCUGAUGAUAAGGAU | AUCCUUAUCAUCAGAAGAA | [430-448](19/19) |
| 131 | Human | 19 | CUUCUUCUGAUGAUAAGGA | UCCUUAUCAUCAGAAGAAG | [429-447](19/19) |
| 132 | Human | 19 | GCAGACCUUUAUGUAUUAU | AUAAUACAUAAAGGUCUGC | [1267-1285](19/19) |
| 133 | Human | 19 | GAGCAGACCUUUAUGUAUU | AAUACAUAAAGGUCUGCUC | [1265-1283](19/19) |
| 134 | Human | 19 | GUGAGCAGACCUUUAUGUA | UACAUAAAGGUCUGCUCAC | [1263-1281](19/19) |
| 135 | Human | 19 | AGUGAGCAGACCUUUAUGU | ACAUAAAGGUCUGCUCACU | [1262-1280](19/19) |
| 136 | Human | 19 | GAAGAUGAGUCGAGUGAGC | GCUCACUCGACUCAUCUUC | [1250-1268](19/19) |
| 137 | Human | 19 | UGAAGUUUCUUCUUCUGAU | AUCAGAAGAAGAAACUUCA | [421-439](19/19) |
| 138 | Human | 19 | CAGUUAAUAUCAUUGCCAA | UUGGCAAUGAUAUUAACUG | [1194-1212](19/19) |
| 139 | Human | 19 | GCAGUUAAUAUCAUUGCCA | UGGCAAUGAUAUUAACUGC | [1193-1211](19/19) |
| 140 | Human | 19 | GCUUGCAGUUAAUAUCAUU | AAUGAUAUUAACUGCAAGC | [1189-1207](19/19) |
| 141 | Human | 19 | CGCUUGCAGUUAAUAUCAU | AUGAUAUUAACUGCAAGCG | [1188-1206](19/19) |
| 142 | Human | 19 | CACGCUUGCAGUUAAUAUC | GAUAUUAACUGCAAGCGUG | [1186-1204](19/19) |
| 143 | Human | 19 | UUUCACGCUUGCAGUUAAU | AUUAACUGCAAGCGUGAAA | [1183-1201](19/19) |
| 144 | Human | 19 | UCAGCUUUCACGCUUGCAG | CUGCAAGCGUGAAAGCUGA | [1178-1196](19/19) |
| 145 | Human | 19 | AUACUAUGUUGCAUCAGCU | AGCUGAUGCAACAUAGUAU | [1165-1183](19/19) |
| 146 | Human | 19 | CAGAUACUAUGUUGCAUCA | UGAUGCAACAUAGUAUCUG | [1162-1180](19/19) |
| 147 | Human | 19 | GCAGAUACUAUGUUGCAUC | GAUGCAACAUAGUAUCUGC | [1161-1179](19/19) |
| 148 | Human | 19 | CCGGCAGAUACUAUGUUGC | GCAACAUAGUAUCUGCCGG | [1158-1176](19/19) |
| 149 | Human | 19 | GCCCGGCAGAUACUAUGUU | AACAUAGUAUCUGCCGGGC | [1156-1174](19/19) |
| 150 | Human | 19 | CUUUGGUAAUGAAGAGUUU | AAACUCUUCAUUACCAAAG | [343-361](19/19) |
| 151 | Human | 19 | GUCAGACUCUGGAGUGAGA | UCUCACUCCAGAGUCUGAC | [1126-1144](19/19) |

FOR 21
STRETCHES:

| | | | | | |
|---|---|---|---|---|---|
| 1 | Cross Sp | 21 | GGAGUCCAGAUGAUGACUUUU | AAAAGUCAUCAUCUGGACUCC | [713-733](21/21) |
| 2 | Cross Sp | 21 | GGAUUGCCACUGAUGAUUCCA | UGGAAUCAUCAGUGGCAAUCC | [795-815](21/21) |
| 3 | Cross Sp | 21 | CUGAAAUGCAUGUGGGUGAUU | AAUCACCCACAUGCAUUUCAG | [1452-1472](21/21) |
| 4 | Cross Sp | 21 | CUGCCUGAAAUGCAUGUGGGU | ACCCACAUGCAUUUCAGGCAG | [1448-1468](21/21) |
| 5 | Cross Sp | 21 | GGAGUGAGAAUCAUAGCUGAG | CUCAGCUAUGAUUCUCACUCC | [1136-1156](21/21) |
| 6 | Cross Sp | 21 | CUCUGGAGUGAGAAUCAUAGC | GCUAUGAUUCUCACUCCAGAG | [1132-1152](21/21) |
| 7 | Cross Sp | 21 | CAAGACUGAAAUACAGUUGGU | ACCAACUGUAUUUCAGUCUUG | [607-627](21/21) |
| 8 | Cross Sp | 21 | GACUCUGGAGUGAGAAUCAUA | UAUGAUUCUCACUCCAGAGUC | [1130-1150](21/21) |
| 9 | Cross Sp | 21 | GCAAGACUGAAAUACAGUUGG | CCAACUGUAUUUCAGUCUUGC | [606-626](21/21) |
| 10 | Cross Sp | 21 | GCAUGUGGGUGAUUGGAUGCU | AGCAUCCAAUCACCCACAUGC | [1459-1479](21/21) |
| 11 | Cross Sp | 21 | AAAUGCAUGUGGGUGAUUGGA | UCCAAUCACCCACAUGCAUUU | [1455-1475](21/21) |
| 12 | Cross Sp | 21 | GAAAUGCAUGUGGGUGAUUGG | CCAAUCACCCACAUGCAUUUC | [1454-1474](21/21) |
| 13 | Cross Sp | 21 | CCUGAAAUGCAUGUGGGUGAU | AUCACCCACAUGCAUUUCAGG | [1451-1471](21/21) |
| 14 | Cross Sp | 21 | CUGGAGUGAGAAUCAUAGCUG | CAGCUAUGAUUCUCACUCCAG | [1134-1154](21/21) |
| 15 | Cross Sp | 21 | UUGCCACUGAUGAUUCCAAAG | CUUUGGAAUCAUCAGUGGCAA | [798-818](21/21) |
| 16 | Cross Sp | 21 | AUUGCCACUGAUGAUUCCAAA | UUUGGAAUCAUCAGUGGCAAU | [797-817](21/21) |
| 17 | Cross Sp | 21 | AGUCCAGAUGAUGACUUUUGA | UCAAAAGUCAUCAUCUGGACU | [715-735](21/21) |
| 18 | Cross Sp | 21 | ACUGAAAUACAGUUGGUGCAG | CUGCACCAACUGUAUUUCAGU | [611-631](21/21) |
| 19 | Cross Sp | 21 | GACUGAAAUACAGUUGGUGCA | UGCACCAACUGUAUUUCAGUC | [610-630](21/21) |
| 20 | Cross Sp | 21 | UGCAUGUGGGUGAUUGGAUGC | GCAUCCAAUCACCCACAUGCA | [1458-1478](21/21) |
| 21 | Cross Sp | 21 | AGACUGAAAUACAGUUGGUGC | GCACCAACUGUAUUUCAGUCU | [609-629](21/21) |
| 22 | Cross Sp | 21 | AAUGCAUGUGGGUGAUUGGAU | AUCCAAUCACCCACAUGCAUU | [1456-1476](21/21) |
| 23 | Cross Sp | 21 | AAGACUGAAAUACAGUUGGUG | CACCAACUGUAUUUCAGUCUU | [608-628](21/21) |
| 24 | Cross Sp | 21 | UGGAGUGAGAAUCAUAGCUGA | UCAGCUAUGAUUCUCACUCCA | [1135-1155](21/21) |
| 25 | Cross Sp | 21 | UCUGGAGUGAGAAUCAUAGCU | AGCUAUGAUUCUCACUCCAGA | [1133-1153](21/21) |
| 26 | Cross Sp | 21 | CGGAUUGCCACUGAUGAUUCC | GGAAUCAUCAGUGGCAAUCCG | [794-814](21/21) |
| 27 | Cross Sp | 21 | AGCAAGACUGAAAUACAGUUG | CAACUGUAUUUCAGUCUUGCU | [605-625](21/21) |
| 28 | Cross Sp | 21 | AUGCAUGUGGGUGAUUGGAUG | CAUCCAAUCACCCACAUGCAU | [1457-1477](21/21) |
| 29 | Cross Sp | 21 | UGAAAUGCAUGUGGGUGAUUG | CAAUCACCCACAUGCAUUUCA | [1453-1473](21/21) |
| 30 | Cross Sp | 21 | GCCUGAAAUGCAUGUGGGUGA | UCACCCACAUGCAUUUCAGGC | [1450-1470](21/21) |
| 31 | Cross Sp | 21 | UGCCUGAAAUGCAUGUGGGUG | CACCCACAUGCAUUUCAGGCA | [1449-1469](21/21) |
| 32 | Cross Sp | 21 | GAGUGAGAAUCAUAGCUGAGC | GCUCAGCUAUGAUUCUCACUC | [1137-1157](21/21) |
| 33 | Cross Sp | 21 | ACUCUGGAGUGAGAAUCAUAG | CUAUGAUUCUCACUCCAGAGU | [1131-1151](21/21) |
| 34 | Cross Sp | 21 | UGCCACUGAUGAUUCCAAAGC | GCUUUGGAAUCAUCAGUGGCA | [799-819](21/21) |
| 35 | Cross Sp | 21 | GAUUGCCACUGAUGAUUCCAA | UUGGAAUCAUCAGUGGCAAUC | [796-816](21/21) |
| 36 | Cross Sp | 21 | GAGUCCAGAUGAUGACUUUUG | CAAAAGUCAUCAUCUGGACUC | [714-734](21/21) |
| 37 | Cross Sp | 21 | AGUGAGAAUCAUAGCUGAGCC | GGCUCAGCUAUGAUUCUCACU | [1138-1158](21/21) |
| 38 | Cross Sp | 21 | GGGACCAACAUGUGAUGGCCU | AGGCCAUCACAUGUUGGUCCC | [1402-1422](21/21) |
| 39 | Cross Sp | 21 | CCUGCCUGAAAUGCAUGUGGG | CCCACAUGCAUUUCAGGCAGG | [1447-1467](21/21) |
| 40 | Cross Sp | 21 | UGGGGACCAACAUGUGAUGGC | GCCAUCACAUGUUGGUCCCCA | [1400-1420](21/21) |
| 41 | Human | 21 | CCAGCGUUGGACAAAUACUUU | AAAGUAUUUGUCCAACGCUGG | [1103-1123](21/21) |
| 42 | Human | 21 | CCAGAGCACAUCCCAAAGCAA | UUGCUUUGGGAUGUGCUCUGG | [762-782](21/21) |
| 43 | Human | 21 | GAAGUUGAGUUGAUGAAAGUU | AACUUUCAUCAACUCAACUUC | [740-760](21/21) |
| 44 | Human | 21 | GGAUUAUCUAUGCAAAUCCUU | AAGGAUUUGCAUAGAUAAUCC | [654-674](21/21) |
| 45 | Human | 21 | CUAGCAAGACUGAAAUACAGU | ACUGUAUUUCAGUCUUGCUAG | [603-623](21/21) |
| 46 | Human | 21 | CUACCGGGACAGGAUUUGACU | AGUCAAAUCCUGUCCCGGUAG | [579-599](21/21) |
| 47 | Human | 21 | GGACCAUGUAACUUAAUUACU | AGUAAUUAAGUUACAUGGUCC | [1776-1796](21/21) |
| 48 | Human | 21 | GCACUCUGGUAGCUGUUAACU | AGUUAACAGCUACCAGAGUGC | [1724-1744](21/21) |
| 49 | Human | 21 | CCAGAUGAGAAGUAUUAUUCA | UGAAUAAUACUUCUCAUCUGG | [1370-1390](21/21) |
| 50 | Human | 21 | GCACAUGUAAAGCCCCUUCUG | CAGAAGGGGCUUUACAUGUGC | [1334-1354](21/21) |
| 51 | Human | 21 | CUCUAUGACCACGCACAUGUA | UACAUGUGCGUGGUCAUAGAG | [1322-1342](21/21) |
| 52 | Human | 21 | GCAGACCUUUAUGUAUUAUGU | ACAUAAUACAUAAAGGUCUGC | [1267-1287](21/21) |
| 53 | Human | 21 | GAGUGAGCAGACCUUUAUGUA | UACAUAAAGGUCUGCUCACUC | [1261-1281](21/21) |
| 54 | Human | 21 | GAGUCGAGUGAGCAGACCUUU | AAAGGUCUGCUCACUCGACUC | [1256-1276](21/21) |
| 55 | Human | 21 | CGAAGAUGAGUCGAGUGAGCA | UGCUCACUCGACUCAUCUUCG | [1249-1269](21/21) |
| 56 | Human | 21 | UGAAGUUUCUUCUUCUGAUGA | UCAUCAGAAGAAGAAACUUCA | [421-441](21/21) |
| 57 | Human | 21 | UCACGCUUGCAGUUAAUAUCA | UGAUAUUAACUGCAAGCGUGA | [1185-1205](21/21) |
| 58 | Human | 21 | GCAUCAGCUUUCACGCUUGCA | UGCAAGCGUGAAAGCUGAUGC | [1175-1195](21/21) |
| 59 | Human | 21 | GCAGAUACUAUGUUGCAUCAG | CUGAUGCAACAUAGUAUCUGC | [1161-1181](21/21) |
| 60 | Human | 21 | UGAGCCCGGCAGAUACUAUGU | ACAUAGUAUCUGCCGGGCUCA | [1153-1173](21/21) |
| 61 | Human | 21 | CGUCAGACUCUGGAGUGAGAA | UUCUCACUCCAGAGUCUGACG | [1125-1145](21/21) |
| 62 | Human | 21 | GGAUCUGAGGAUGUGAAACUU | AAGUUUCACAUCCUCAGAUCC | [1052-1072](21/21) |
| 63 | Human | 21 | CCUGGAUCUGAGGAUGUGAAA | UUUCACAUCCUCAGAUCCAGG | [1049-1069](21/21) |
| 64 | Human | 21 | GGACCAGAAAAUUAAUGAAGU | ACUUCAUUAAUUUUCUGGUCC | [406-426](21/21) |
| 65 | Human | 21 | GGUUGGUUUCAGCAUGUAUCU | AGAUACAUGCUGAAACCAACC | [1006-1026](21/21) |
| 66 | Human | 21 | GACAUUCUGGACCAGAAAAUU | AAUUUUCUGGUCCAGAAUGUC | [398-418](21/21) |
| 67 | Human | 21 | GAGCUAAAUAUCGAUGUUGUU | AACAACAUCGAUAUUUAGCUC | [890-910](21/21) |
| 68 | Human | 21 | GAACGGGCGAAAGAGCUAAAU | AUUUAGCUCUUUCGCCCGUUC | [878-898](21/21) |
| 69 | Human | 21 | CAGAACCAGCAGGCUCCUUUU | AAAAGGAGCCUGCUGGUUCUG | [856-876](21/21) |
| 70 | Human | 21 | CCAGAUGAUGACUUUUGAUAG | CUAUCAAAAGUCAUCAUCUGG | [718-738](21/21) |
| 71 | Human | 21 | GUCCAGAUGAUGACUUUUGAU | AUCAAAAGUCAUCAUCUGGAC | [716-736](21/21) |
| 72 | Human | 21 | CUAAUAAUGGAGUCCAGAUGA | UCAUCUGGACUCCAUUAUUAG | [705-725](21/21) |
| 73 | Human | 21 | GAGAGGAUUAUCUAUGCAAAU | AUUUGCAUAGAUAAUCCUCUC | [650-670](21/21) |
| 74 | Human | 21 | CCAGAGAGGAUUAUCUAUGCA | UGCAUAGAUAAUCCUCUCUGG | [647-667](21/21) |
| 75 | Human | 21 | GCCUCCAGAGAGGAUUAUCUA | UAGAUAAUCCUCUCUGGAGGC | [643-663](21/21) |
| 76 | Human | 21 | GACUGCCACUUCCUCGAUGAA | UUCAUCGAGGAAGUGGCAGUC | [362-382](21/21) |
| 77 | Human | 21 | GGACAGGAUUUGACUGUGCUA | UAGCACAGUCAAAUCCUGUCC | [585-605](21/21) |
| 78 | Human | 21 | GAACAACUUUGGUAAUGAAGA | UCUUCAUUACCAAAGUUGUUC | [337-357](21/21) |
| 79 | Human | 21 | CCUUUUAUGCAGUCAAAUGUA | UACAUUUGACUGCAUAAAAGG | [525-545](21/21) |
| 80 | Human | 21 | CCCCCUUUUAUGCAGUCAAAU | AUUUGACUGCAUAAAAGGGGG | [522-542](21/21) |
| 81 | Human | 21 | GCAGAAUGGGCCAAAAGCUUA | UAAGCUUUUGGCCCAUUCUGC | [1996-2016](21/21) |
| 82 | Human | 21 | GUACAAUGGCAGAAUGGGCCA | UGGCCCAUUCUGCCAUUGUAC | [1988-2008](21/21) |
| 83 | Human | 21 | CAAGCAUUUGUAGCUUGUACA | UGUACAAGCUACAAAUGCUUG | [1972-1992](21/21) |
| 84 | Human | 21 | CCUCAGCUGCUGAACAAGCAU | AUGCUUGUUCAGCAGCUGAGG | [1958-1978](21/21) |
| 85 | Human | 21 | CUCUUCAGACACGCUACUCAA | UUGAGUAGCGUGUCUGAAGAG | [1930-1950](21/21) |
| 86 | Human | 21 | GGAUUUUUAUUCACUCUUCAG | CUGAAGAGUGAAUAAAAAUCC | [1917-1937](21/21) |
| 87 | Human | 21 | CUAGGAUAUGGGUCACACUUA | UAAGUGUGACCCAUAUCCUAG | [1855-1875](21/21) |
| 88 | Human | 21 | GAAACAUCUGAGGUGGUUAAA | UUUAACCACCUCAGAUGUUUC | [484-504](21/21) |
| 89 | Human | 21 | GCCAUAUGGAAGACUAGGAUA | UAUCCUAGUCUUCCAUAUGGC | [1842-1862](21/21) |
| 90 | Human | 21 | CCAUGAUGCAGCCAUAUGGAA | UUCCAUAUGGCUGCAUCAUGG | [1832-1852](21/21) |
| 91 | Human | 21 | GCCAUGAUGCAGCCAUAUGGA | UCCAUAUGGCUGCAUCAUGGC | [1831-1851](21/21) |
| 92 | Human | 21 | GUCGCCAUGAUGCAGCCAUAU | AUAUGGCUGCAUCAUGGCGAC | [1828-1848](21/21) |
| 93 | Human | 21 | CUGCUAGUUUUGAAAUGUCUU | AAGACAUUUCAAAACUAGCAG | [1795-1815](21/21) |
| 94 | Human | 21 | GGGGGGACCAUGUAACUUAAU | AUUAAGUUACAUGGUCCCCCC | [1772-1792](21/21) |
| 95 | Human | 21 | GGAUUUGGGGGGACCAUGUAA | UUACAUGGUCCCCCCAAAUCC | [1766-1786](21/21) |
| 96 | Human | 21 | CUGCAAGUUUAGCUUGAAUUA | UAAUUCAAGCUAAACUUGCAG | [1743-1763](21/21) |
| 97 | Human | 21 | GUAGCUGUUAACUGCAAGUUU | AAACUUGCAGUUAACAGCUAC | [1732-1752](21/21) |
| 98 | Human | 21 | GGAGACAUUCUAAAGAAACAU | AUGUUUCUUUAGAAUGUCUCC | [470-490](21/21) |
| 99 | Human | 21 | CAGACCUGGGAGACAUUCUAA | UUAGAAUGUCUCCCAGGUCUG | [462-482](21/21) |
| 100 | Human | 21 | CAACUCAUGCAGCAAUUCCAG | CUGGAAUUGCUGCAUGAGUUG | [1577-1597](21/21) |
| 101 | Human | 21 | CUACGUUCAAUGGCUUCCAGA | UCUGGAAGCCAUUGAACGUAG | [1518-1538](21/21) |
| 102 | Human | 21 | GGAUGCUCUUUGAAAACAUGG | CCAUGUUUUCAAAGAGCAUCC | [1473-1493](21/21) |
| 103 | Human | 21 | GGUGAUUGGAUGCUCUUUGAA | UUCAAAGAGCAUCCAAUCACC | [1466-1486](21/21) |
| 104 | Human | 21 | CAACAUGUGAUGGCCUCGAUC | GAUCGAGGCCAUCACAUGUUG | [1407-1427](21/21) |
| 105 | Human | 21 | GCAUAUGGGGACCAACAUGUG | CACAUGUUGGUCCCCAUAUGC | [1395-1415](21/21) |
| 106 | Human | 21 | CCAGCAUAUGGGGACCAACAU | AUGUUGGUCCCCAUAUGCUGG | [1392-1412](21/21) |
| 107 | Human | 21 | GAAGUAUUAUUCAUCCAGCAU | AUGCUGGAUGAAUAAUACUUC | [1378-1398](21/21) |
| 108 | Human | 21 | GACCUAAACCAGAUGAGAAGU | ACUUCUCAUCUGGUUUAGGUC | [1362-1382](21/21) |
| 109 | Human | 21 | AGAGACCUAAACCAGAUGAGA | UCUCAUCUGGUUUAGGUCUCU | [1359-1379](21/21) |
| 110 | Human | 21 | CUAUGACCACGCACAUGUAAA | UUUACAUGUGCGUGGUCAUAG | [1324-1344](21/21) |
| 111 | Human | 21 | AUACUCUAUGACCACGCACAU | AUGUGCGUGGUCAUAGAGUAU | [1319-1339](21/21) |
| 112 | Human | 21 | GCAUACUCUAUGACCACGCAC | GUGCGUGGUCAUAGAGUAUGC | [1317-1337](21/21) |
| 113 | Human | 21 | CGUCUAUGGAUCAUUUAAUUG | CAAUUAAAUGAUCCAUAGACG | [1297-1317](21/21) |
| 114 | Human | 21 | GCGUCUAUGGAUCAUUUAAUU | AAUUAAAUGAUCCAUAGACGC | [1296-1316](21/21) |
| 115 | Human | 21 | AUGGCGUCUAUGGAUCAUUUA | UAAAUGAUCCAUAGACGCCAU | [1293-1313](21/21) |
| 116 | Human | 21 | UGAAUGAUGGCGUCUAUGGAU | AUCCAUAGACGCCAUCAUUCA | [1287-1307](21/21) |
| 117 | Human | 21 | GUGAAUGAUGGCGUCUAUGGA | UCCAUAGACGCCAUCAUUCAC | [1286-1306](21/21) |
| 118 | Human | 21 | CUUCUUCUGAUGAUAAGGAUG | CAUCCUUAUCAUCAGAAGAAG | [429-449](21/21) |
| 119 | Human | 21 | CAGACCUUUAUGUAUUAUGUG | CACAUAAUACAUAAAGGUCUG | [1268-1288](21/21) |
| 120 | Human | 21 | GAGCAGACCUUUAUGUAUUAU | AUAAUACAUAAAGGUCUGCUC | [1265-1285](21/21) |
| 121 | Human | 21 | UGAGCAGACCUUUAUGUAUUA | UAAUACAUAAAGGUCUGCUCA | [1264-1284](21/21) |
| 122 | Human | 21 | CGAGUGAGCAGACCUUUAUGU | ACAUAAAGGUCUGCUCACUCG | [1260-1280](21/21) |
| 123 | Human | 21 | GACGAAGAUGAGUCGAGUGAG | CUCACUCGACUCAUCUUCGUC | [1247-1267](21/21) |
| 124 | Human | 21 | CUGAUGACGAAGAUGAGUCGA | UCGACUCAUCUUCGUCAUCAG | [1242-1262](21/21) |
| 125 | Human | 21 | GCUCUGAUGACGAAGAUGAGU | ACUCAUCUUCGUCAUCAGAGC | [1239-1259](21/21) |
| 126 | Human | 21 | GGCUCUGAUGACGAAGAUGAG | CUCAUCUUCGUCAUCAGAGCC | [1238-1258](21/21) |
| 127 | Human | 21 | GAAGUUUCUUCUUCUGAUGAU | AUCAUCAGAAGAAGAAACUUC | [422-442](21/21) |
| 128 | Human | 21 | GCAGUUAAUAUCAUUGCCAAG | CUUGGCAAUGAUAUUAACUGC | [1193-1213](21/21) |
| 129 | Human | 21 | CUUUCACGCUUGCAGUUAAUA | UAUUAACUGCAAGCGUGAAAG | [1182-1202](21/21) |
| 130 | Human | 21 | GCUUUCACGCUUGCAGUUAAU | AUUAACUGCAAGCGUGAAAGC | [1181-1201](21/21) |
| 131 | Human | 21 | CAGCUUUCACGCUUGCAGUUA | UAACUGCAAGCGUGAAAGCUG | [1179-1199](21/21) |
| 132 | Human | 21 | AUACUAUGUUGCAUCAGCUUU | AAAGCUGAUGCAACAUAGUAU | [1165-1185](21/21) |
| 133 | Human | 21 | CCCGGCAGAUACUAUGUUGCA | UGCAACAUAGUAUCUGCCGGG | [1157-1177](21/21) |
| 134 | Human | 21 | CAGACUCUGGAGUGAGAAUCA | UGAUUCUCACUCCAGAGUCUG | [1128-1148](21/21) |
| 135 | Human | 21 | GGACAAAUACUUUCCGUCAGA | UCUGACGGAAAGUAUUUGUCC | [1111-1131](21/21) |

| | | | | |
|---|---|---|---|---|
| | | Oligo | | |
| No | Source | Length | mouse 7305336 | rat 6981301 |

FOR 19
STRETCHES:

| | | | | |
|---|---|---|---|---|
| 1 | Cross Sp | 19 | [1862-1880](19/19) | [1548-1566](19/19) |
| 2 | Cross Sp | 19 | [1117-1135](19/19) | [803-821](19/19) |
| 3 | Cross Sp | 19 | [1116-1134](19/19) | [802-820](19/19) |
| 4 | Cross Sp | 19 | [1009-1027](19/19) | [695-713](19/19) |
| 5 | Cross Sp | 19 | [1015-1033](19/19) | [701-719](19/19) |
| 6 | Cross Sp | 19 | [1540-1558](19/19) | [1226-1244](19/19) |
| 7 | Cross Sp | 19 | [1539-1557](19/19) | [1225-1243](19/19) |
| 8 | Cross Sp | 19 | [1204-1222](19/19) | [890-908](19/19) |
| 9 | Cross Sp | 19 | [1202-1220](19/19) | [888-906](19/19) |
| 10 | Cross Sp | 19 | [1010-1028](19/19) | [696-714](19/19) |
| 11 | Cross Sp | 19 | [1197-1215](19/19) | [883-910](19/19) |
| 12 | Cross Sp | 19 | [1120-1138](19/19) | [806-824](19/19) |
| 13 | Cross Sp | 19 | [1013-1031](19/19) | [699-717](19/19) |
| 14 | Cross Sp | 19 | [1856-1874](19/19) | [1542-1560](19/19) |
| 15 | Cross Sp | 19 | [1855-1873](19/19) | [1541-1559](19/19) |
| 16 | Cross Sp | 19 | [1853-1871](19/19) | [1539-1557](19/19) |
| 17 | Cross Sp | 19 | [1851-1869](19/19) | [1537-1555](19/19) |
| 18 | Cross Sp | 19 | [1012-1030](19/19) | [698-716](19/19) |
| 19 | Cross Sp | 19 | [1803-1821](19/19) | [1489-1507](19/19) |
| 20 | Cross Sp | 19 | [1538-1556](19/19) | [1224-1242](19/19) |
| 21 | Cross Sp | 19 | [1536-1554](19/19) | [1222-1240](19/19) |
| 22 | Cross Sp | 19 | [1535-1553](19/19) | [1221-1239](19/19) |
| 23 | Cross Sp | 19 | [1534-1552](19/19) | [1220-1238](19/19) |
| 24 | Cross Sp | 19 | [1533-1551](19/19) | [1219-1237](19/19) |
| 25 | Cross Sp | 19 | [1201-1219](19/19) | [887-905](19/19) |
| 26 | Cross Sp | 19 | [1199-1217](19/19) | [885-903](19/19) |
| 27 | Cross Sp | 19 | [1198-1216](19/19) | [884-902](19/19) |
| 28 | Cross Sp | 19 | [1119-1137](19/19) | [805-823](19/19) |
| 29 | Cross Sp | 19 | [1063-1081](19/19) | [749-767](19/19) |
| 30 | Cross Sp | 19 | [1062-1080](19/19) | [748-766](19/19) |
| 31 | Cross Sp | 19 | [1861-1879](19/19) | [1547-1565](19/19) |
| 32 | Cross Sp | 19 | [1860-1878](19/19) | [1546-1564](19/19) |
| 33 | Cross Sp | 19 | [1859-1877](19/19) | [1545-1563](19/19) |
| 34 | Cross Sp | 19 | [1858-1876](19/19) | [1544-1562](19/19) |
| 35 | Cross Sp | 19 | [1857-1875](19/19) | [1543-1561](19/19) |
| 36 | Cross Sp | 19 | [1852-1870](19/19) | [1538-1556](19/19) |
| 37 | Cross Sp | 19 | [1850-1868](19/19) | [1536-1554](19/19) |
| 38 | Cross Sp | 19 | [1807-1825](19/19) | [1493-1511](19/19) |
| 39 | Cross Sp | 19 | [1543-1561](19/19) | [1229-1247](19/19) |
| 40 | Cross Sp | 19 | [1537-1555](19/19) | [1223-1241](19/19) |
| 41 | Cross Sp | 19 | [1011-1029](19/19) | [697-715](19/19) |
| 42 | Cross Sp | 19 | [1203-1221](19/19) | [889-907](19/19) |
| 43 | Cross Sp | 19 | [1061-1079](19/19) | [747-765](19/19) |
| 44 | Cross Sp | 19 | [1008-1026](19/19) | [694-712](19/19) |
| 45 | Cross Sp | 19 | [1060-1078](19/19) | [746-764](19/19) |
| 46 | Cross Sp | 19 | [1864-1882](19/19) | [1550-1568](19/19) |
| 47 | Cross Sp | 19 | [1863-1881](19/19) | [1549-1567](19/19) |
| 48 | Cross Sp | 19 | [1542-1560](19/19) | [1228-1246](19/19) |
| 49 | Cross Sp | 19 | [1200-1218](19/19) | [886-904](19/19) |
| 50 | Cross Sp | 19 | [1118-1136](19/19) | [804-822](19/19) |
| 51 | Cross Sp | 19 | [1016-1034](19/19) | [702-720](19/19) |
| 52 | Cross Sp | 19 | [1014-1032](19/19) | [700-718](19/19) |
| 53 | Cross Sp | 19 | [1854-1872](19/19) | [1540-1558](19/19) |
| 54 | Cross Sp | 19 | [1541-1559](19/19) | [1227-1245](19/19) |
| 55 | Cross Sp | 19 | [1806-1824](19/19) | [1492-1510](19/19) |
| 56 | Cross Sp | 19 | [1805-1823](19/19) | [1491-1509](19/19) |
| 57 | Cross Sp | 19 | [1804-1822](19/19) | [1490-1508](19/19) |
| 58 | Human | 19 | — | — |
| 59 | Human | 19 | [1420-1438](18/19) | [1106-1121](19/19) |
| 60 | Human | 19 | [1182-1193](12/12) | [868-881](14/14) |
| 61 | Human | 19 | — | — |
| 62 | Human | 19 | — | — |
| 63 | Human | 19 | [2263-2274](12/12) | [1954-1965](12/12) |
| 64 | Human | 19 | — | — |
| 65 | Human | 19 | — | — |
| 66 | Human | 19 | [1950-1960](11/11) | — |
| 67 | Human | 19 | — | — |
| 68 | Human | 19 | — | — |
| 69 | Human | 19 | — | — |
| 70 | Human | 19 | — | [511-529](18/19) |
| 71 | Human | 19 | — | — |
| 72 | Human | 19 | — | — |
| 73 | Human | 19 | — | — |
| 74 | Human | 19 | — | — |
| 75 | Human | 19 | — | — |
| 76 | Human | 19 | [802-820](18/19) | [488-506](18/19) |
| 77 | Human | 19 | [800-814](15/15) | [486-500](15/15) |
| 78 | Human | 19 | [1290-1303](14/14) | [976-989](14/14) |
| 79 | Human | 19 | [1285-1303](18/19) | [971-989](18/19) |
| 80 | Human | 19 | [1262-1273](12/12) | [948-959](12/12) |
| 81 | Human | 19 | [1262-1273](12/12) | [948-959](12/12) |
| 82 | Human | 19 | [790-808](18/19) | [478-494](16/17) |
| 83 | Human | 19 | [1166-1177](12/12) | [852-863](12/12) |
| 84 | Human | 19 | — | [827-845](18/19) |
| 85 | Human | 19 | [1127-1145](18/19) | [813-831](18/19) |
| 86 | Human | 19 | [1122-1138](17/17) | [808-824](17/17) |
| 87 | Human | 19 | — | [462-476](15/15) |
| 88 | Human | 19 | — | [461-476](16/16) |
| 89 | Human | 19 | [1068-1086](18/19) | [754-772](18/19) |
| 90 | Human | 19 | [1064-1081](18/18) | [750-767](18/18) |
| 91 | Human | 19 | [1054-1072](18/19) | [740-758](19/19) |
| 92 | Human | 19 | — | [773-751](19/19) |
| 93 | Human | 19 | [1003-1021](18/19) | [689-707](18/19) |
| 94 | Human | 19 | — | — |
| 95 | Human | 19 | — | — |
| 96 | Human | 19 | — | — |
| 97 | Human | 19 | — | — |
| 98 | Human | 19 | [2393-2411](18/19) | [2079-2097](18/19) |
| 99 | Human | 19 | [2375-2389](15/15) | [2058-2075](18/18) |
| 100 | Human | 19 | — | — |
| 101 | Human | 19 | [2282-2293](12/12) | [1973-1984](12/12 |
| 102 | Human | 19 | [2277-2293](17/17) | [1968-1984](17/17) |
| 103 | Human | 19 | [891-902](12/12) | [577-588](12/12) |
| 104 | Human | 19 | — | — |
| 105 | Human | 19 | — | — |
| 106 | Human | 19 | — | [1866-1884](18/19) |
| 107 | Human | 19 | — | — |
| 108 | Human | 19 | [2148-2166](19/19) | [1834-1848](15/15) |
| 109 | Human | 19 | [2146-2156](11/11) | [1832-1842](11/11) |
| 110 | Human | 19 | — | — |
| 111 | Human | 19 | [873-889](17/17) | — |
| 112 | Human | 19 | [867-884](17/18) | — |
| 113 | Human | 19 | [1978-1988](11/11) | [1664-1680](16/17) |
| 114 | Human | 19 | [1950-1960](11/11) | — |
| 115 | Human | 19 | — | — |
| 116 | Human | 19 | [1870-1888](18/19) | [1556-1574](18/19) |
| 117 | Human | 19 | [1849-1865](17/17) | [1536-1551](16/16) |
| 118 | Human | 19 | [1843-1861](18/19) | [1536-1547](12/12) |
| 119 | Human | 19 | [843-860](17/18) | [528-546](18/19) |
| 120 | Human | 19 | [1773-1789](16/17) | [1459-1475](17/17) |
| 121 | Human | 19 | — | — |
| 122 | Human | 19 | — | — |
| 123 | Human | 19 | — | — |
| 124 | Human | 19 | [1707-1723](16/17) | — |
| 125 | Human | 19 | [1704-1717](14/14) | — |
| 126 | Human | 19 | [1704-1716](13/13) | — |
| 127 | Human | 19 | [1704-1715](12/12) | — |
| 128 | Human | 19 | — | — |
| 129 | Human | 19 | [1688-1699](12/12) | [1375-1385](11/11) |
| 130 | Human | 19 | — | [523-537](15/15) |
| 131 | Human | 19 | — | [518-536](18/19) |
| 132 | Human | 19 | — | — |
| 133 | Human | 19 | — | — |
| 134 | Human | 19 | — | — |
| 135 | Human | 19 | — | — |
| 136 | Human | 19 | [1653-1663](11/11) | [1339-1349](11/11) |
| 137 | Human | 19 | — | [510-528](18/19) |
| 138 | Human | 19 | [1605-1615](11/11) | [1283-1301](18/19) |
| 139 | Human | 19 | — | [1282-1300](18/19) |
| 140 | Human | 19 | — | — |
| 141 | Human | 19 | — | — |
| 142 | Human | 19 | [1589-1600](12/12) | — |
| 143 | Human | 19 | [1586-1600](15/15) | — |
| 144 | Human | 19 | [1581-1599](19/19) | [1267-1285](18/19) |
| 145 | Human | 19 | — | — |
| 146 | Human | 19 | [1565-1583](18/19) | — |
| 147 | Human | 19 | [1564-1582](18/19) | — |
| 148 | Human | 19 | [1563-1576](14/14) | [1249-1259](11/11) |
| 149 | Human | 19 | [1563-1576](14/14) | [1249-1259](11/11) |
| 150 | Human | 19 | — | — |
| 151 | Human | 19 | [1530-1547](18/18) | [1219-1233](15/15) |

FOR 21
STRETCHES:

| | | | | |
|---|---|---|---|---|
| 1 | Cross Sp | 21 | [1116-1136](21/21) | [802-822](21/21) |
| 2 | Cross Sp | 21 | [1198-1218](21/21) | [884-904](21/21) |
| 3 | Cross Sp | 21 | [1855-1875](21/21) | [1541-1561](21/21) |
| 4 | Cross Sp | 21 | [1851-1871](21/21) | [1537-1557](21/21) |
| 5 | Cross Sp | 21 | [1539-1559](21/21) | [1225-1245](21/21) |
| 6 | Cross Sp | 21 | [1535-1555](21/21) | [1221-1241](21/21) |
| 7 | Cross Sp | 21 | [1010-1030](21/21) | [696-716](21/21) |
| 8 | Cross Sp | 21 | [1533-1553](21/21) | [1219-1239](21/21) |
| 9 | Cross Sp | 21 | [1009-1029](21/21) | [695-715](21/21) |
| 10 | Cross Sp | 21 | [1862-1882](21/21) | [1548-1568](21/21) |
| 11 | Cross Sp | 21 | [1858-1878](21/21) | [1544-1564](21/21) |
| 12 | Cross Sp | 21 | [1857-1877](21/21) | [1543-1563](21/21) |
| 13 | Cross Sp | 21 | [1854-1874](21/21) | [1540-1560](21/21) |
| 14 | Cross Sp | 21 | [1537-1557](21/21) | [1223-1243](21/21) |
| 15 | Cross Sp | 21 | [1201-1221](21/21) | [887-907](21/21) |
| 16 | Cross Sp | 21 | [1200-1220](21/21) | [886-906](21/21) |
| 17 | Cross Sp | 21 | [1118-1138](21/21) | [804-824](21/21) |
| 18 | Cross Sp | 21 | [1014-1034](21/21) | [700-720](21/21) |
| 19 | Cross Sp | 21 | [1013-1033](21/21) | [699-719](21/21) |
| 20 | Cross Sp | 21 | [1861-1881](21/21) | [1547-1567](21/21) |
| 21 | Cross Sp | 21 | [1012-1032](21/21) | [698-718](21/21) |
| 22 | Cross Sp | 21 | [1859-1879](21/21) | [1545-1565](21/21) |
| 23 | Cross Sp | 21 | [1011-1031](21/21) | [697-717](21/21) |
| 24 | Cross Sp | 21 | [1538-1558](21/21) | [1224-1244](21/21) |
| 25 | Cross Sp | 21 | [1536-1556](21/21) | [1222-1242](21/21) |
| 26 | Cross Sp | 21 | [1197-1217](21/21) | [883-903](21/21) |
| 27 | Cross Sp | 21 | [1008-1028](21/21) | [694-714](21/21) |
| 28 | Cross Sp | 21 | [1860-1880](21/21) | [1546-1566](21/21) |
| 29 | Cross Sp | 21 | [1856-1876](21/21) | [1542-1562](21/21) |
| 30 | Cross Sp | 21 | [1853-1873](21/21) | [1539-1559](21/21) |
| 31 | Cross Sp | 21 | [1852-1872](21/21) | [1538-1558](21/21) |
| 32 | Cross Sp | 21 | [1540-1560](21/21) | [1226-1246](21/21) |
| 33 | Cross Sp | 21 | [1534-1554](21/21) | [1220-1240](21/21) |
| 34 | Cross Sp | 21 | [1202-1222](21/21) | [888-908](21/21) |
| 35 | Cross Sp | 21 | [1199-1219](21/21) | [885-905](21/21) |
| 36 | Cross Sp | 21 | [1117-1137](21/21) | [803-823](21/21) |
| 37 | Cross Sp | 21 | [1541-1561](21/21) | [1227-1247](21/21) |
| 38 | Cross Sp | 21 | [1805-1825](21/21) | [1491-1511](21/21) |
| 39 | Cross Sp | 21 | [1850-1870](21/21) | [1536-1556](21/21) |
| 40 | Cross Sp | 21 | [1803-1823](21/21) | [1489-1509](21/21) |
| 41 | Human | 21 | — | — |
| 42 | Human | 21 | [1165-1177](13/13) | [851-863](13/13) |
| 43 | Human | 21 | [1147-1162](15/16) | [833-849](17/17) |
| 44 | Human | 21 | [1060-1077](18/18) | [743-763](21/21) |
| 45 | Human | 21 | [1008-1026](19/19) | [694-712](19/19) |
| 46 | Human | 21 | [987-1002](16/16) | [673-686](14/14) |
| 47 | Human | 21 | — | [1866-1886](20/21) |
| 48 | Human | 21 | — | — |
| 49 | Human | 21 | [1773-1789](16/17) | [1459-1475](17/17) |
| 50 | Human | 21 | — | — |
| 51 | Human | 21 | — | — |
| 52 | Human | 21 | [1674-1690](16/17) | — |
| 53 | Human | 21 | — | — |
| 54 | Human | 21 | — | — |
| 55 | Human | 21 | [1653-1663](11/11) | [1339-1349](11/11) |
| 56 | Human | 21 | — | [510-530](20/21) |
| 57 | Human | 21 | [1588-1600](13/13) | — |
| 58 | Human | 21 | [1578-1598](21/21) | [1264-1284](20/21) |
| 59 | Human | 21 | [1564-1584](20/21) | — |
| 60 | Human | 21 | [1556-1576](20/21) | [1242-1259](17/18) |
| 61 | Human | 21 | [1530-1548](19/19) | [1219-1234](16/16) |
| 62 | Human | 21 | — | — |
| 63 | Human | 21 | [1452-1462](11/11) | — |
| 64 | Human | 21 | — | — |
| 65 | Human | 21 | [1410-1429](19/20) | [1096-1115](20/20) |
| 66 | Human | 21 | [801-820](19/20) | [487-506](19/20) |
| 67 | Human | 21 | [1293-1303](11/11) | [979-995](16/17) |
| 68 | Human | 21 | [1281-1301](20/21) | [967-987](20/21) |
| 69 | Human | 21 | [1262-1273](12/12) | [948-959](12/12) |
| 70 | Human | 21 | [1121-1138](18/18) | [807-824](18/18) |
| 71 | Human | 21 | [1119-1138](20/20) | [805-824](20/20) |
| 72 | Human | 21 | [1116-1128](13/13) | [798-814](17/17) |
| 73 | Human | 21 | [1053-1073](20/21) | [739-759](21/21) |
| 74 | Human | 21 | [1051-1070](19/20) | [736-756](21/21) |
| 75 | Human | 21 | — | [732-752](21/21) |
| 76 | Human | 21 | — | [451-471](19/21) |
| 77 | Human | 21 | [988-1006](19/19) | [674-692](18/19) |
| 78 | Human | 21 | — | — |
| 79 | Human | 21 | — | — |
| 80 | Human | 21 | — | — |
| 81 | Human | 21 | [2393-2413](20/21) | [2079-2099](20/21) |
| 82 | Human | 21 | [2385-2405](20/21) | [2071-2089](18/19) |
| 83 | Human | 21 | [2369-2389](20/21) | [2055-2075](21/21) |
| 84 | Human | 21 | [2355-2373](18/19) | — |
| 85 | Human | 21 | — | — |
| 86 | Human | 21 | [2313-2333](20/21) | [2000-2020](20/21) |
| 87 | Human | 21 | [2263-2274](12/12) | [1954-1965](12/12) |
| 88 | Human | 21 | [891-907](16/17) | [577-588](12/12) |
| 89 | Human | 21 | — | — |
| 90 | Human | 21 | — | — |
| 91 | Human | 21 | — | — |
| 92 | Human | 21 | — | — |
| 93 | Human | 21 | — | — |
| 94 | Human | 21 | [2175-2193](18/19) | [1862-1882](20/21) |
| 95 | Human | 21 | [2171-2185](15/15) | [1858-1872](15/15) |
| 96 | Human | 21 | [2146-2166](21/21) | [1832-1848](17/17) |
| 97 | Human | 21 | — | — |
| 98 | Human | 21 | [873-889](17/17) | — |
| 99 | Human | 21 | [867-885](18/19) | — |
| 100 | Human | 21 | — | — |
| 101 | Human | 21 | — | — |
| 102 | Human | 21 | — | — |
| 103 | Human | 21 | [1869-1888](19/20) | [1555-1574](19/20) |
| 104 | Human | 21 | [1810-1830](20/21) | [1496-1516](20/21) |
| 105 | Human | 21 | [1798-1818](20/21) | [1484-1504](20/21) |
| 106 | Human | 21 | [1795-1815](20/21) | [1481-1501](20/21) |
| 107 | Human | 21 | [1781-1801](20/21) | [1467-1487](20/21) |
| 108 | Human | 21 | — | [1459-1471](13/13) |
| 109 | Human | 21 | — | — |
| 110 | Human | 21 | — | — |
| 111 | Human | 21 | — | — |
| 112 | Human | 21 | — | — |
| 113 | Human | 21 | [1704-1717](14/14) | — |
| 114 | Human | 21 | [1704-1717](14/14) | — |
| 115 | Human | 21 | [1704-1716](13/13) | — |
| 116 | Human | 21 | — | — |
| 117 | Human | 21 | [1689-1709](19/21) | [1375-1394](18/20) |
| 118 | Human | 21 | — | [518-538](20/21) |
| 119 | Human | 21 | [1674-1691](17/18) | — |
| 120 | Human | 21 | — | — |
| 121 | Human | 21 | — | — |
| 122 | Human | 21 | — | — |
| 123 | Human | 21 | [1653-1663](11/11) | [1339-1349](11/11) |
| 124 | Human | 21 | [1653-1663](11/11) | [1339-1349](11/11) |
| 125 | Human | 21 | — | — |
| 126 | Human | 21 | — | — |
| 127 | Human | 21 | — | [511-531](20/21) |
| 128 | Human | 21 | [1596-1615](18/20) | [1282-1301](19/20) |
| 129 | Human | 21 | [1585-1600](16/16) | — |
| 130 | Human | 21 | [1584-1600](17/17) | — |
| 131 | Human | 21 | [1582-1600](19/19) | — |
| 132 | Human | 21 | [1568-1588](20/21) | [1254-1274](19/21) |
| 133 | Human | 21 | [1563-1576](14/14) | [1249-1259](11/11) |
| 134 | Human | 21 | [1531-1551](21/21) | [1219-1237](19/19) |
| 135 | Human | 21 | — | — |

Table H below shows further siRNA compounds specific for the PIM1 gene that have been selected according to the present invention.

TABLE H

| | | | | | |
|---|---|---|---|---|---|
| | | Oligo | | | |
| No | Source | Length | Sense siRNA | AntiSense siRNA | human 31543400 |

FOR 19
STRETCHES:

| | | | | | |
|---|---|---|---|---|---|
| 1 | Cross Sp | 19 | AGAAGGACCGGAUUUCCGA | UCGGAAAUCCGGUCCUUCU | [572-590](19/19) |
| 2 | Cross Sp | 19 | GUGGAGAAGGACCGGAUUU | AAAUCCGGUCCUUCUCCAC | [568-586](19/19) |
| 3 | Cross Sp | 19 | UGGAGAAGGACCGGAUUUC | GAAAUCCGGUCCUUCUCCA | [569-587](19/19) |
| 4 | Cross Sp | 19 | GAGAAGGACCGGAUUUCCG | CGGAAAUCCGGUCCUUCUC | [571-589](19/19) |
| 5 | Cross Sp | 19 | GGAGAAGGACCGGAUUUCC | GGAAAUCCGGUCCUUCUCC | [570-588](19/19) |
| 6 | Cross Sp | 19 | ACCGCGACAUCAAGGACGA | UCGUCCUUGAUGUCGCGGU | [857-875](19/19) |
| 7 | Cross Sp | 19 | UCCACCGCGACAUCAAGGA | UCCUUGAUGUCGCGGUGGA | [854-872](19/19) |
| 8 | Cross Sp | 19 | CGUGGAGAAGGACCGGAUU | AAUCCGGUCCUUCUCCACG | [567-585](19/19) |
| 9 | Cross Sp | 19 | ACGUGGAGAAGGACCGGAU | AUCCGGUCCUUCUCCACGU | [566-584](19/19) |
| 10 | Cross Sp | 19 | UGCCCAUGGAAGUGGUCCU | AGGACCACUUCCAUGGGCA | [620-638](19/19) |
| 11 | Cross Sp | 19 | GAGUGCCCAUGGAAGUGGU | ACCACUUCCAUGGGCACUC | [617-635](19/19) |
| 12 | Cross Sp | 19 | UCUUCUGGCAGGUGCUGGA | UCCAGCACCUGCCAGAAGA | [803-821](19/19) |
| 13 | Cross Sp | 19 | ACAACUUGCCGGUGGCCAU | AUGGCCACCGGCAAGUUGU | [542-560](19/19) |
| 14 | Cross Sp | 19 | UGGAGUCGCAGUACCAGGU | ACCUGGUACUGCGACUCCA | [464-482](19/19) |
| 15 | Cross Sp | 19 | AGUGCCCAUGGAAGUGGUC | GACCACUUCCAUGGGCACU | [618-636](19/19) |
| 16 | Cross Sp | 19 | UUCUUCUGGCAGGUGCUGG | CCAGCACCUGCCAGAAGAA | [802-820](19/19) |
| 17 | Human | 19 | GGAUCCUGCUGUAUGAUAU | AUAUCAUACAGCAGGAUCC | [1049-1067](19/19) |
| 18 | Human | 19 | GGAGGUCAAUGUUAUGUAU | AUACAUAACAUUGACCUCC | [2190-2208](19/19) |
| 19 | Human | 19 | CAACAGAAAAGCUGUAAAU | AUUUACAGCUUUUCUGUUG | [2048-2066](19/19) |
| 20 | Human | 19 | CAAGACCUCACACACACAA | UUGUGUGUGUGAGGUCUUG | [2008-2026](19/19) |
| 21 | Human | 19 | GAACUUGCCUCUUUUACCU | AGGUAAAAGAGGCAAGUUC | [1788-1806](19/19) |
| 22 | Human | 19 | GAACUGUUUUCUUCAUCAU | AUGAUGAAGAAAACAGUUC | [1621-1639](19/19) |
| 23 | Human | 19 | CCAGGAAACUGCUGAGAUC | GAUCUCAGCAGUUUCCUGG | [1251-1269](19/19) |
| 24 | Human | 19 | CCAGAACCAUCCAUGGAUG | CAUCCAUGGAUGGUUCUGG | [1215-1233](19/19) |
| 25 | Human | 19 | CAACCUUCGAAGAAAUCCA | UGGAUUUCUUCGAAGGUUG | [1199-1217](19/19) |
| 26 | Human | 19 | GGCCAACCUUCGAAGAAAU | AUUUCUUCGAAGGUUGGCC | [1196-1214](19/19) |
| 27 | Human | 19 | CCAUCAGAUAGGCCAACCU | AGGUUGGCCUAUCUGAUGG | [1186-1204](19/19) |
| 28 | Human | 19 | CAGAAUGUCAGCAUCUCAU | AUGAGAUGCUGACAUUCUG | [1145-1163](19/19) |
| 29 | Human | 19 | CAGAGGGUCUCUUCAGAAU | AUUCUGAAGAGACCCUCUG | [1132-1150](19/19) |
| 30 | Human | 19 | UCAGGGGCCAGGUUUUCUU | AAGAAAACCUGGCCCCUGA | [1109-1127](19/19) |
| 31 | Human | 19 | GGUGUGUGGAGAUAUUCCU | AGGAAUAUCUCCACACACC | [1068-1086](19/19) |
| 32 | Human | 19 | CAAGGACGAAAACAUCCUU | AAGGAUGUUUUCGUCCUUG | [867-885](19/19) |
| 33 | Human | 19 | ACAUCAAGGACGAAAACAU | AUGUUUUCGUCCUUGAUGU | [863-881](19/19) |
| 34 | Human | 19 | GCGACAUCAAGGACGAAAA | UUUUCGUCCUUGAUGUCGC | [860-878](19/19) |
| 35 | Human | 19 | GUAGCCUGCUGGUUUUAUC | GAUAAAACCAGCAGGCUAC | [2537-2555](19/19) |
| 36 | Human | 19 | CAGGUAGCCUGGUGGUUUU | AAAACCAGCAGGCUACCUG | [2534-2552](19/19) |
| 37 | Human | 19 | GGAUAAUUAUGAAAAGCAG | CUGCUUUUCAUAAUUAUCC | [2465-2483](19/19) |
| 38 | Human | 19 | GCAUUGCUGACUGUGUAUA | UAUACACAGUCAGCAAUGC | [2444-2462](19/19) |
| 39 | Human | 19 | GGGCAUUGCUGACUGUGUA | UACACAGUCAGCAAUGCCC | [2442-2460](19/19) |
| 40 | Human | 19 | CCAUCAAACACGUGGAGAA | UUCUCCACGUGUUUGAUGG | [557-575](19/19) |
| 41 | Human | 19 | CCUAUUCCAAGCUUCCAUA | UAUGGAAGCUUGGAAUAGG | [2236-2254](19/19) |
| 42 | Human | 19 | CUAACCUGGAGGUCAAUGU | ACAUUGACCUCCAGGUUAG | [2183-2201](19/19) |
| 43 | Human | 19 | AGAUUGUAGUGGAUCUAAU | AUUAGAUCCACUACAAUCU | [2098-2116](19/19) |
| 44 | Human | 19 | GCAUGGUAGUAUACAAAAA | UUUUUGUAUACUACCAUGC | [2080-2098](19/19) |
| 45 | Human | 19 | CAGUUGGCAUGGUAGUAUA | UAUACUACCAUGCCAACUG | [2074-2092](19/19) |
| 46 | Human | 19 | GUGUACAGUUGGCAUGGUA | UACCAUGCCAACUGUACAC | [2069-2087](19/19) |
| 47 | Human | 19 | GCAAUCAACAGAAAAGCUG | CAGCUUUUCUGUUGAUUGC | [2043-2061](19/19) |
| 48 | Human | 19 | ACAAUGCAAUCAACAGAAA | UUUCUGUUGAUUGCAUUGU | [2038-2056](19/19) |
| 49 | Human | 19 | GCACAAACAAUGCAAUCAA | UUGAUUGCAUUGUUUGUGC | [2032-2050](19/19) |
| 50 | Human | 19 | CACAAAAAAUGCACAAACA | UGUUUGUGCAUUUUUUGUG | [2022-2040](19/19) |
| 51 | Human | 19 | AGACCUCACACACACAAAA | UUUUGUGUGUGUGAGGUCU | [2010-2028](19/19) |
| 52 | Human | 19 | GUGAACUGGUCUUCCUUUU | AAAAGGAAGACCAGUUCAC | [1963-1981](19/19) |
| 53 | Human | 19 | CGAGUGAACUGGUCUUCCU | AGGAAGACCAGUUCACUCG | [1960-1978](19/19) |
| 54 | Human | 19 | CCUUUUUUCUGCCUCCUUU | AAAGGAGGCAGAAAAAAGG | [1931-1949](19/19) |
| 55 | Human | 19 | UGAGCCACCUGCCCUUUUU | AAAAAGGGCAGGUGGCUCA | [1919-1937](19/19) |
| 56 | Human | 19 | CCCCCUCCUUCAUAUGAAA | UUUCAUAUGAAGGAGGGGG | [1868-1886](19/19) |
| 57 | Human | 19 | CUCACCCCCUCCUUCAUAU | AUAUGAAGGAGGGGGUGAG | [1864-1882](19/19) |
| 58 | Human | 19 | GGGUUUUGUUCCCUAUUUU | AAAAUAGGGAACAAAACCC | [1830-1848](19/19) |
| 59 | Human | 19 | CCAAAAAUCUGCCUGGGUU | AACCAGGCAGAUUUUUGG | [1816-1834](19/19) |
| 60 | Human | 19 | CCUGCUGCUUCUCCAAAAA | UUUUUGGAGAAGCAGCAGG | [1804-1822](19/19) |
| 61 | Human | 19 | GGGAGAAAUACUUGAACUU | AAGUUCAAGUAUUUCUCCC | [1775-1793](19/19) |
| 62 | Human | 19 | GGAUAGGACUAGCACCAUU | AAUGGUGCUAGUCCUAUCC | [1693-1711](19/19) |
| 63 | Human | 19 | GGAUGGGAUAGGACUAGCA | UGCUAGUCCUAUCCCAUCC | [1688-1706](19/19) |
| 64 | Human | 19 | GGAACUGUUUUCUUCAUCA | UGAUGAAGAAAACAGUUCC | [1620-1638](19/19) |
| 65 | Human | 19 | CCAUGGAACUGUUUUCUUC | GAAGAAAACAGUUCCAUGG | [1616-1634](19/19) |
| 66 | Human | 19 | UCAGAACCCUGCCAUGGAA | UUCCAUGGCAGGGUUCUGA | 1605-1623](19/19) |
| 67 | Human | 19 | CUCUCUUCUUCUCAUAGGU | ACCUAUGAGAAGAAGAGAG | [1534-1552](19/19) |
| 68 | Human | 19 | AUACUCUCUUCUUCUCAUA | UAUGAGAAGAAGAGAGUAU | [1531-1549](19/19) |
| 69 | Human | 19 | CCCAUAGAUACUCUCUUCU | AGAAGAGAGUAUCUAUGGG | [1524-1542](19/19) |
| 70 | Human | 19 | CUCAACUCCUCCCAUAGAU | AUCUAUGGGAGGAGUUGAG | [1514-1532](19/19) |
| 71 | Human | 19 | ACAUUUACAACUCAUUCCA | UGGAAUGAGUUGUAAAUGU | [1430-1448](19/19) |
| 72 | Human | 19 | GAACAACAUUUACAACUCA | UGAGUUGUAAAUGUUGUUC | [1425-1443](19/19) |
| 73 | Human | 19 | ACAGGAACAACAUUUACAA | UUGUAAAUGUUGUUCCUGU | [1421-1439](19/19) |
| 74 | Human | 19 | GCUUGAUACAGGAACAACA | UGUUGUUCCUGUAUCAAGC | [1414-1432](19/19) |
| 75 | Human | 19 | GCAGGACAGUGCUUGAUAC | GUAUCAAGCACUGUCCUGC | [1404-1422](19/19) |
| 76 | Human | 19 | GAGUACCAGUGACACGUCU | AGACGUGUCACUGGUACUC | [1379-1397](19/19) |
| 77 | Human | 19 | AAACUGCUGAGAUCCACCU | AGGUGGAUCUCAGCAGUUU | [1256-1274](19/19) |
| 78 | Human | 19 | GGAAACUGCUGAGAUCCAC | GUGGAUCUCAGCAGUUUCC | [1254-1272](19/19) |
| 79 | Human | 19 | GGAUGCAAGAUGUUCUCCU | AGGAGAACAUCUUGCAUCC | [1229-1247](19/19) |
| 80 | Human | 19 | CAUGGAUGCAAGAUGUUCU | AGAACAUCUUGCAUCCAUG | [1226-1244](19/19) |
| 81 | Human | 19 | AUCCAUGGAUGCAAGAUGU | ACAUCUUGCAUCCAUGGAU | [1223-1241](19/19) |
| 82 | Human | 19 | GAACCAUCCAUGGAUGCAA | UUGCAUCCAUGGAUGGUUC | [1218-1236](19/19) |
| 83 | Human | 19 | GAAAUCCAGAACCAUCCAU | AUGGAUGGUUCUGGAUUUC | [1210-1228](19/19) |
| 84 | Human | 19 | CGAAGAAAUCCAGAACCAU | AUGGUUCUGGAUUUCUUCG | [1206-1224](19/19) |
| 85 | Human | 19 | AGAUAGGCCAACCUUCGAA | UUCGAAGGUUGGCCUAUCU | [1191-1209](19/19) |
| 86 | Human | 19 | CAUCAGAUAGGCCAACCUU | AAGGUUGGCCUAUCUGAUG | [1187-1205](19/19) |
| 87 | Human | 19 | GAGACCAUCAGAUAGGCCA | UGGCCUAUCUGAUGGUCUC | [1182-1200](19/19) |
| 88 | Human | 19 | GCCCUGAGACCAUCAGAUA | UAUCUGAUGGUCUCAGGGC | [1177-1195](19/19) |
| 89 | Human | 19 | CAUCUCAUUAGAUGGUGCU | AGCACCAUCUAAUGAGAUG | [1156-1174](19/19) |
| 90 | Human | 19 | GUCAGCAUCUCAUUAGAUG | CAUCUAAUGAGAUGCUGAC | [1151-1169](19/19) |
| 91 | Human | 19 | AUGUCAGCAUCUCAUUAGA | UCUAAUGAGAUGCUGACAU | [1149-1167](19/19) |
| 92 | Human | 19 | AGAAUGUCAGCAUCUCAUU | AAUGAGAUGCUGACAUUCU | [1146-1164](19/19) |
| 93 | Human | 19 | GGUCUCUUCAGAAUGUCAG | CUGACAUUCUGAAGAGACC | [1137-1155](19/19) |
| 94 | Human | 19 | GAGGGUCUCUUCAGAAUGU | ACAUUCUGAAGAGACCCUC | [1134-1152](19/19) |
| 95 | Human | 19 | GCAGAGGGUCUCUUCAGAA | UUCUGAAGAGACCCUCUGC | [1131-1149](19/19) |
| 96 | Human | 19 | GCCAGGUUUUCUUCAGGCA | UGCCUGAAGAAAACCUGGC | [1115-1133](19/19) |
| 97 | Human | 19 | UCAUCAGGGGCCAGGUUUU | AAAACCUGGCCCCUGAUGA | [1106-1124](19/19) |
| 98 | Human | 19 | GCAUGACGAAGAGAUCAUC | GAUGAUCUCUUCGUCAUGC | [1092-1110](19/19) |
| 99 | Human | 19 | CGAGCAUGACGAAGAGAUC | GAUCUCUUCGUCAUGCUCG | [1089-1107](19/19) |
| 100 | Human | 19 | UCGAGCAUGACGAAGAGAU | AUCUCUUCGUCAUGCUCGA | [1088-1106](19/19) |
| 101 | Human | 19 | GUGGAGAUAUUCCUUUCGA | UCGAAAGGAAUAUCUCCAC | [1073-1091](19/19) |
| 102 | Human | 19 | UAUGGUGUGUGGAGAUAUU | AAUAUCUCCACACACCAUA | [1065-1083](19/19) |
| 103 | Human | 19 | CCUGCUGUAUGAUAUGGUG | CACCAUAUCAUACAGCAGG | [1053-1071](19/19) |
| 104 | Human | 19 | UGGGGAUCCUGCUGUAUGA | UCAUACAGCAGGAUCCCCA | [1046-1064](19/19) |
| 105 | Human | 19 | AGAGUGGAUCCGCUACCAU | AUGGUAGCGGAUCCACUCU | [993-1011](19/19) |
| 106 | Human | 19 | CGUCUACACGGACUUCGAU | AUCGAAGUCCGUGUAGACG | [951-969](19/19) |
| 107 | Human | 19 | ACACCGUCUACACGGACUU | AAGUCCGUGUAGACGGUGU | [947-965](19/19) |
| 108 | Human | 19 | CUCAAGGACACCGUCUACA | UGUAGACGGUGUCCUUGAG | [940-958](19/19) |
| 109 | Human | 19 | CAUCCUUAUCGACCUCAAU | AUUGAGGUCGAUAAGGAUG | [879-897](19/19) |
| 110 | Human | 19 | ACAUCCUUAUCGACCUCAA | UUGAGGUCGAUAAGGAUGU | [878-896](19/19) |
| 111 | Human | 19 | CGAAAACAUCCUUAUCGAC | GUCGAUAAGGAUGUUUUCG | [873-891](19/19) |
| 112 | Human | 19 | ACGAAAACAUCCUUAUCGA | UCGAUAAGGAUGUUUUCGU | [872-890](19/19) |
| 113 | Human | 19 | AGGACGAAAACAUCCUUAU | AUAAGGAUGUUUUCGUCCU | [869-887](19/19) |
| 114 | Human | 19 | CGACAUCAAGGACGAAAAC | GUUUUCGUCCUUGAUGUCG | [861-879](19/19) |
| 115 | Human | 19 | AAGAUCUCUUCGACCUCAU | AUGAAGUCGAAGAGAUCUU | [743-761](19/19) |
| 116 | Human | 19 | CAAGAUCUCUUCGACUUCA | UGAAGUCGAAGAGAUCUUG | [742-760](19/19) |

FOR 21
STRETCHES:

| | | | | | |
|---|---|---|---|---|---|
| 1 | Cross Sp | 21 | ACGUGGAGAAGGACCGGAUUU | AAAUCCGGUCCUUCUCCACGU | [566-586](21/21) |
| 2 | Cross Sp | 21 | GGAGAAGGACCGGAUUUCCGA | UCGGAAAUCCGGUCCUUCUCC | [570-590](21/21) |
| 3 | Cross Sp | 21 | CACGUGGAGAAGGACCGGAUU | AAUCCGGUCCUUCUCCACGUG | [565-585](21/21) |
| 4 | Cross Sp | 21 | GUGGAGAAGGACCGGAUUUCC | GGAAAUCCGGUCCUUCUCCAC | [568-588](21/21) |
| 5 | Cross Sp | 21 | CGUGGAGAAGGACCGGAUUUC | GAAAUCCGGUCCUUCUCCACG | [567-587](21/21) |
| 6 | Cross Sp | 21 | UGGAGAAGGACCGGAUUUCCG | CGGAAAUCCGGUCCUUCUCCA | [569-589](21/21) |
| 7 | Cross Sp | 21 | UUCUUCUGGCAGGUGCUGGAG | CUCCAGCACCUGCCAGAAGAA | [802-822](21/21) |
| 8 | Cross Sp | 21 | AGUGCCCAUGGAAGUGGUCCU | AGGACCACUUCCAUGGGCACU | [618-638](21/21) |
| 9 | Human | 21 | GCAGGUAGCCUGCUGGUUUUA | UAAAACCAGCAGGCUACCUGC | [2533-2553](21/21) |
| 10 | Human | 21 | CCAUAGCUGCUGCCCUAGUUU | AAACUAGGGCAGCAGCUAUGG | [2250-2270](21/21) |
| 11 | Human | 21 | GCACAAACAAUGCAAUCAACA | UGUUGAUUGCAUUGUUUGUGC | [2032-2052](21/21) |
| 12 | Human | 21 | CCAAGACCUCACACACACAAA | UUUGUGUGUGUGAGGUCUUGG | [2007-2027](21/21) |
| 13 | Human | 21 | GAACUGGUCUUCCUUUUUGGU | ACCAAAAAGGAAGACCAGUUC | [1965-1985](21/21) |
| 14 | Human | 21 | GGAUAGGACUAGCACCAUUUU | AAAAUGGUGCUAGUCCUAUCC | [1693-1713](21/21) |
| 15 | Human | 21 | GCAUUGCUGGACUCUGAAAUA | UAUUUCAGAGUCCAGCAAUGC | [1558-1578](21/21) |
| 16 | Human | 21 | CCAGCAUUGCUGGACUCUGAA | UUCAGAGUCCAGCAAUGCUGG | [1555-1575](21/21) |
| 17 | Human | 21 | CCAUAGAUACUCUCUUCUUCU | AGAAGAAGAGAGUAUCUAUGG | [1525-1545](21/21) |
| 18 | Human | 21 | GAAGCUUCUGUCUCCAGCUUC | GAAGCUGGAGACAGAAGCUUC | [1356-1376](21/21) |
| 19 | Human | 21 | CAUCCAUGGAUGCAAGAUGUU | AACAUCUUGCAUCCAUGGAUG | [1222-1242](21/21) |
| 20 | Human | 21 | CCAUCCAUGGAUGCAAGAUGU | ACAUCUUGCAUCCAUGGAUGG | [1221-1241](21/21) |
| 21 | Human | 21 | GAACCAUCCAUGGAUGCAAGA | UCUUGCAUCCAUGGAUGGUUC | [1218-1238](21/21) |
| 22 | Human | 21 | GAAGAAAUCCAGAACCAUCCA | UGGAUGGUUCUGGAUUUCUUC | [1207-1227](21/21) |
| 23 | Human | 21 | CAACCUUCGAAGAAAUCCAGA | UCUGGAUUUCUUCGAAGGUUG | [1199-1219](21/21) |
| 24 | Human | 21 | GCAUCUCAUUAGAUGGUGCUU | AAGCACCAUCUAAUGAGAUGC | [1155-1175](21/21) |
| 25 | Human | 21 | UCAGAAUGUCAGCAUCUCAUU | AAUGAGAUGCUGACAUUCUGA | [1144-1164](21/21) |
| 26 | Human | 21 | GCAGAGGGUCUCUUCAGAAUG | CAUUCUGAAGAGACCCUCUGC | [1131-1151](21/21) |
| 27 | Human | 21 | CUGGGGAUCCUGCUGUAUGAU | AUCAUACAGCAGGAUCCCCAG | [1045-1065](21/21) |
| 28 | Human | 21 | GGACGAAAACAUCCUUAUCGA | UCGAUAAGGAUGUUUUCGUCC | [870-890](21/21) |
| 29 | Human | 21 | CAAGGACGAAAACAUCCUUAU | AUAAGGAUGUUUUCGUCCUUG | [867-887](21/21) |
| 30 | Human | 21 | CAAGAUCUCUUCGACUUCAUC | GAUGAAGUCGAAGAGAUCUUG | [742-762](21/21) |
| 31 | Human | 21 | GAAGAAGGUGAGCUCGGGUUU | AAACCCGAGCUCACCUUCUUC | [642-662](21/21) |
| 32 | Human | 21 | GAAGUGGUCCUGCUGAAGAAG | CUUCUUCAGCAGGACCACUUC | [628-648](21/21) |
| 33 | Human | 21 | GUACAGGGGAAUAAAAGAGAU | AUCUCUUUUAUUCCCCUGUAC | [2569-2589](21/21) |
| 34 | Human | 21 | GGUAGCCUGCUGGUUUUAUCU | AGAUAAAACCAGCAGGCUACC | [2536-2556](21/21) |
| 35 | Human | 21 | GGGGCAUUGCUGACUGUGUAU | AUACACAGUCAGCAAUGCCCC | [2441-2461](21/21) |
| 36 | Human | 21 | CCUCCUUUCCUCCUCUGACUU | AAGUCAGAGGAGGAAAGGAGG | [2276-2296](21/21) |
| 37 | Human | 21 | GCAUGGUAGUAUACAAAAAGA | UCUUUUUGUAUACUACCAUGC | [2080-2100](21/21) |
| 38 | Human | 21 | ACAGAAAAGCUGUAAAUGUGU | ACACAUUUACAGCUUUUCUGU | [2050-2070](21/21) |
| 39 | Human | 21 | GACCUCACACACACAAAAAAU | AUUUUUUGUGUGUGUGAGGUC | [2011-2031](21/21) |
| 40 | Human | 21 | CGAGUGAACUGGUCUUCCUUU | AAAGGAAGACCAGUUCACUCG | [1960-1980](21/21) |
| 41 | Human | 21 | CCGAGUGAACUGGUCUUCCUU | AAGGAAGACCAGUUCACUCGG | [1959-1979](21/21) |
| 42 | Human | 21 | CCCUUUUUUCUGCCUCCUUUA | UAAAGGAGGCAGAAAAAAGGG | [1930-1950](21/21) |
| 43 | Human | 21 | CUGAGCCACCUGCCCUUUUUU | AAAAAAGGGCAGGUGGCUCAG | [1918-1938](21/21) |
| 44 | Human | 21 | GCCUGGGUUUUGUUCCCUAUU | AAUAGGGAACAAAACCCAGGC | [1826-1846](21/21) |
| 45 | Human | 21 | CCAAAAAUCUGCCUGGGUUUU | AAAACCCAGGCAGAUUUUUGG | [1816-1836](21/21) |
| 46 | Human | 21 | CUGCUGCUUCUCCAAAAAUCU | AGAUUUUUGGAGAAGCAGCAG | [1805-1825](21/21) |
| 47 | Human | 21 | GGAACUGUUUUCUUCAUCAUG | CAUGAUGAAGAAAACAGUUCC | [1620-1640](21/21) |
| 48 | Human | 21 | GCCAUGGAACUGUUUUCUUCA | UGAAGAAAACAGUUCCAUGGC | [1615-1635](21/21) |
| 49 | Human | 21 | AUAGAUACUCUCUUCUUCUCA | UGAGAAGAAGAGAGUAUCUAU | [1527-1547](21/21) |
| 50 | Human | 21 | CCUCAACUCCUCCCAUAGAUA | UAUCUAUGGGAGGAGUUGAGG | [1513-1533](21/21) |
| 51 | Human | 21 | CAACAUUUACAACUCAUUCCA | UGGAAUGAGUUGUAAAUGUUG | [1428-1448](21/21) |
| 52 | Human | 21 | GGAACAACAUUUACAACUCAU | AUGAGUUGUAAAUGUUGUUCC | [1424-1444](21/21) |
| 53 | Human | 21 | GGACAGUGCUUGAUACAGGAA | UUCCUGUAUCAAGCACUGUCC | [1407-1427](21/21) |
| 54 | Human | 21 | CAAGCAGGACAGUGCUUGAUA | UAUCAAGCACUGUCCUGCUUG | [1401-1421](21/21) |
| 55 | Human | 21 | UCCUCCCCUCUCUUGUCAGAU | AUCUGACAAGAGAGGGGAGGA | [1322-1342](21/21) |
| 56 | Human | 21 | GGAUGCAAGAUGUUCUCCUGC | GCAGGAGAACAUCUUGCAUCC | [1229-1249](21/21) |
| 57 | Human | 21 | CAUGGAUGCAAGAUGUUCUCC | GGAGAACAUCUUGCAUCCAUG | [1226-1246](21/21) |
| 58 | Human | 21 | CCAUGGAUGCAAGAUGUUCUC | GAGAACAUCUUGCAUCCAUGG | [1225-1245](21/21) |
| 59 | Human | 21 | CCAGAACCAUCCAUGGAUGCA | UGCAUCCAUGGAUGGUUCUGG | [1215-1235](21/21) |
| 60 | Human | 21 | UUCGAAGAAAUCCAGAACCAU | AUGGUUCUGGAUUUCUUCGAA | [1204-1224](21/21) |
| 61 | Human | 21 | CCAACCUUCGAAGAAAUCCAG | CUGGAUUUCUUCGAAGGUUGG | [1198-1218](21/21) |
| 62 | Human | 21 | AGAUAGGCCAACCUUCGAAGA | UCUUCGAAGGUUGGCCUAUCU | [1191-1211](21/21) |
| 63 | Human | 21 | CCAUCAGAUAGGCCAACCUUC | GAAGGUUGGCCUAUCUGAUGG | [1186-1206](21/21) |
| 64 | Human | 21 | ACCAUCAGAUAGGCCAACCUU | AAGGUUGGCCUAUCUGAUGGU | [1185-1205](21/21) |
| 65 | Human | 21 | UGAGACCAUCAGAUAGGCCAA | UUGGCCUAUCUGAUGGUCUCA | [1181-1201](21/21) |
| 66 | Human | 21 | UGGCCCUGAGACCAUCAGAUA | UAUCUGAUGGUCUCAGGGCCA | [1175-1195](21/21) |
| 67 | Human | 21 | AAUGUCAGCAUCUCAUUAGAU | AUCUAAUGAGAUGCUGACAUU | [1148-1168](21/21) |
| 68 | Human | 21 | GAAUGUCAGCAUCUCAUUAGA | UCUAAUGAGAUGCUGACAUUC | [1147-1167](21/21) |
| 69 | Human | 21 | CAGAAUGUCAGCAUCUCAUUA | UAAUGAGAUGCUGACAUUCUG | [1145-1165](21/21) |
| 70 | Human | 21 | CUUCAGAAUGUCAGCAUCUCA | UGAGAUGCUGACAUUCUGAAG | [1142-1162](21/21) |
| 71 | Human | 21 | CUCUUCAGAAUGUCAGCAUCU | AGAUGCUGACAUUCUGAAGAG | [1140-1160](21/21) |
| 72 | Human | 21 | GAGGGUCUCUUCAGAAUGUCA | UGACAUUCUGAAGAGACCCUC | [1134-1154](21/21) |
| 73 | Human | 21 | AGGCAGAGGGUCUCUUCAGAA | UUCUGAAGAGACCCUCUGCCU | [1129-1149](21/21) |
| 74 | Human | 21 | GGUUUUCUUCAGGCAGAGGGU | ACCCUCUGCCUGAAGAAAACC | [1119-1139](21/21) |
| 75 | Human | 21 | UCAGGGGCCAGGUUUUCUUCA | UGAAGAAAACCUGGCCCCUGA | [1109-1129](21/21) |
| 76 | Human | 21 | GAUCAUCAGGGGCCAGGUUUU | AAAACCUGGCCCCUGAUGAUC | [1104-1124](21/21) |
| 77 | Human | 21 | AGAUCAUCAGGGGCCAGGUUU | AAACCUGGCCCCUGAUGAUCU | [1103-1123](21/21) |
| 78 | Human | 21 | CAUGACGAAGAGAUCAUCAGG | CCUGAUGAUCUCUUCGUCAUG | [1093-1113](21/21) |
| 79 | Human | 21 | CUUUCGAGCAUGACGAAGAGA | UCUCUUCGUCAUGCUCGAAAG | [1085-1105](21/21) |
| 80 | Human | 21 | GUGUGGAGAUAUUCCUUUCGA | UCGAAAGGAAUAUCUCCACAC | [1071-1091](21/21) |
| 81 | Human | 21 | UGGUGUGUGGAGAUAUUCCUU | AAGGAAUAUCUCCACACACCA | [1067-1087](21/21) |
| 82 | Human | 21 | UGAUAUGGUGUGUGGAGAUAU | AUAUCUCCACACACCAUAUCA | [1062-1082](21/21) |
| 83 | Human | 21 | CUGUAUGAUAUGGUGUGUGGA | UCCACACACCAUAUCAUACAG | [1057-1077](21/21) |
| 84 | Human | 21 | GGGAUCCUGCUGUAUGAUAUG | CAUAUCAUACAGCAGGAUCCC | [1048-1068](21/21) |
| 85 | Human | 21 | GAGCUCAAGCUCAUCGACUUC | GAAGUCGAUGAGCUUGAGCUC | [904-924](21/21) |
| 86 | Human | 21 | AACAUCCUUAUCGACCUCAAU | AUUGAGGUCGAUAAGGAUGUU | [877-897](21/21) |
| 87 | Human | 21 | GACGAAAACAUCCUUAUCGAC | GUCGAUAAGGAUGUUUUCGUC | [871-891](21/21) |
| 88 | Human | 21 | UCAAGGACGAAAACAUCCUUA | UAAGGAUGUUUUCGUCCUUGA | [866-886](21/21) |
| 89 | Human | 21 | GACAUCAAGGACGAAAACAUC | GAUGUUUUCGUCCUUGAUGUC | [862-882](21/21) |
| 90 | Human | 21 | GCGACAUCAAGGACGAAAACA | UGUUUUCGUCCUUGAUGUCGC | [860-880](21/21) |
| 91 | Human | 21 | CGCGACAUCAAGGACGAAAAC | GUUUUCGUCCUUGAUGUCGCG | [859-879](21/21) |
| 92 | Human | 21 | CUCUUCGACUUCAUCACGGAA | UUCCGUGAUGAAGUCGAAGAG | [748-768](21/21) |
| 93 | Human | 21 | GAUCUCUUCGACUUCAUCACG | CGUGAUGAAGUCGAAGAGAUC | [745-765](21/21) |
| 94 | Human | 21 | GCAAGAUCUCUUCGACUUCAU | AUGAAGUCGAAGAGAUCUUGC | [741-761](21/21) |
| 95 | Human | 21 | GUGCAAGAUCUCUUCGACUUC | GAAGUCGAAGAGAUCUUGCAC | [739-759](21/21) |
| 96 | Human | 21 | CGGUGCAAGAUCUCUUCGACU | AGUCGAAGAGAUCUUGCACCG | [737-757](21/21) |
| 97 | Human | 21 | GACAGUUUCGUCCUGAUCCUG | CAGGAUCAGGACGAAACUGUC | [703-723](21/21) |
| 98 | Human | 21 | GGAAGUGGUCCUGCUGAAGAA | UUCUUCAGCAGGACCACUUCC | [627-647](21/21) |
| 99 | Human | 21 | GGGGAAUAAAAGAGAUCUUAU | AUAAGAUCUCUUUUAUUCCCC | [2574-2594](21/21) |
| 100 | Human | 21 | ACAGGGGAAUAAAAGAGAUCU | AGAUCUCUUUUAUUCCCCUGU | [2571-2591](21/21) |
| 101 | Human | 21 | GAAAUACUGUACAGGGGAAUA | UAUUCCCCUGUACAGUAUUUC | [2561-2581](21/21) |
| 102 | Human | 21 | GCUGGUUUUAUCUGAGUGAAA | UUUCACUCAGAUAAAACCAGC | [2544-2564](21/21) |
| 103 | Human | 21 | GUAGCCUGCUGGUUUUAUCUG | CAGAUAAAACCAGCAGGCUAC | [2537-2557](21/21) |
| 104 | Human | 21 | CUGUGUUUUGAGCAGCAGGUA | UACCUGCUGCUCAAAACACAG | [2519-2539](21/21) |
| 105 | Human | 21 | CAGUUCUGGAUGGUGUGCCUU | AAGGCACACCAUCCAGAACUG | [2481-2501](21/21) |
| 106 | Human | 21 | CUGACUGUGUAUAUAGGAUAA | UUAUCCUAUAUACACAGUCAG | [2450-2470](21/21) |
| 107 | Human | 21 | GCUGACUGUGUAUAUAGGAUA | UAUCCUAUAUACACAGUCAGC | [2449-2469](21/21) |
| 108 | Human | 21 | UUGCUGACUGUGUAUAUAGGA | UCCUAUAUACACAGUCAGCAA | [2447-2467](21/21) |

| | | | | |
|---|---|---|---|---|
| | | Oligo | | |
| No | Source | Length | mouse 40254619 | rat 8393958 |

FOR 19
STRETCHES:

| | | | | |
|---|---|---|---|---|
| 1 | Cross Sp | 19 | [541-559](19/19) | [212-230](19/19) |
| 2 | Cross Sp | 19 | [537-555](19/19) | [208-226](19/19) |
| 3 | Cross Sp | 19 | [538-556](19/19) | [209-227](19/19) |
| 4 | Cross Sp | 19 | [540-558](19/19) | [211-229](19/19) |
| 5 | Cross Sp | 19 | [539-557](19/19) | [210-228](19/19) |
| 6 | Cross Sp | 19 | [826-844](19/19) | [497-515](19/19) |
| 7 | Cross Sp | 19 | [823-841](19/19) | [494-512](19/19) |
| 8 | Cross Sp | 19 | [536-554](19/19) | [207-225](19/19) |
| 9 | Cross Sp | 19 | [535-553](19/19) | [206-224](19/19) |
| 10 | Cross Sp | 19 | [589-607](19/19) | [260-278](19/19) |
| 11 | Cross Sp | 19 | [586-604](19/19) | [257-275](19/19) |
| 12 | Cross Sp | 19 | [772-790](19/19) | [443-461](19/19) |
| 13 | Cross Sp | 19 | [511-529](19/19) | [182-200](19/19) |
| 14 | Cross Sp | 19 | [433-451](19/19) | [104-122](19/19) |
| 15 | Cross Sp | 19 | [587-605](19/19) | [258-276](19/19) |
| 16 | Cross Sp | 19 | [771-789](19/19) | [442-460](19/19) |
| 17 | Human | 19 | — | — |
| 18 | Human | 19 | — | — |
| 19 | Human | 19 | — | — |
| 20 | Human | 19 | [1942-1955](14/14) | — |
| 21 | Human | 19 | — | — |
| 22 | Human | 19 | — | — |
| 23 | Human | 19 | — | — |
| 24 | Human | 19 | [1187-1202](16/16) | [885-873](18/19) |
| 25 | Human | 19 | — | [842-857](15/16) |
| 26 | Human | 19 | — | — |
| 27 | Human | 19 | — | — |
| 28 | Human | 19 | — | — |
| 29 | Human | 19 | [1107-1117](11/11) | [775-790](16/16) |
| 30 | Human | 19 | — | — |
| 31 | Human | 19 | [1044-1054](11/11) | [715-725](11/11) |
| 32 | Human | 19 | — | — |
| 33 | Human | 19 | [832-850](18/19) | [503-521](18/19) |
| 34 | Human | 19 | [829-844](16/16) | [500-515](16/16) |
| 35 | Human | 19 | — | — |
| 36 | Human | 19 | — | — |
| 37 | Human | 19 | — | — |
| 38 | Human | 19 | [64-74](11/11) | — |
| 39 | Human | 19 | [64-74](11/11) | — |
| 40 | Human | 19 | [534-544](11/11) | [197-215](18/19) |
| 41 | Human | 19 | — | — |
| 42 | Human | 19 | — | — |
| 43 | Human | 19 | — | — |
| 44 | Human | 19 | — | — |
| 45 | Human | 19 | — | — |
| 46 | Human | 19 | — | — |
| 47 | Human | 19 | — | — |
| 48 | Human | 19 | — | — |
| 49 | Human | 19 | — | — |
| 50 | Human | 19 | — | — |
| 51 | Human | 19 | [1944-1955](12/12) | — |
| 52 | Human | 19 | [1900-1912](13/13) | — |
| 53 | Human | 19 | [1900-1912](13/13) | — |
| 54 | Human | 19 | — | — |
| 55 | Human | 19 | [1853-1871](18/19) | — |
| 56 | Human | 19 | — | — |
| 57 | Human | 19 | — | — |
| 58 | Human | 19 | — | — |
| 59 | Human | 19 | [1761-1772](12/12) | — |
| 60 | Human | 19 | [1744-1756](13/13) | — |
| 61 | Human | 19 | [1716-1734](19/19) | — |
| 62 | Human | 19 | [1643-1653](11/11) | [1292-1302](11/11) |
| 63 | Human | 19 | — | — |
| 64 | Human | 19 | — | — |
| 65 | Human | 19 | — | — |
| 66 | Human | 19 | — | — |
| 67 | Human | 19 | — | — |
| 68 | Human | 19 | — | — |
| 69 | Human | 19 | — | — |
| 70 | Human | 19 | — | — |
| 71 | Human | 19 | [1373-1385](13/13) | [1034-1046](13/13) |
| 72 | Human | 19 | — | — |
| 73 | Human | 19 | — | — |
| 74 | Human | 19 | — | — |
| 75 | Human | 19 | — | — |
| 76 | Human | 19 | — | — |
| 77 | Human | 19 | — | — |
| 78 | Human | 19 | — | — |
| 79 | Human | 19 | [843-853](11/11) | [869-887](18/19) |
| 80 | Human | 19 | — | — |
| 81 | Human | 19 | [1192-1204](13/13) | — |
| 82 | Human | 19 | [1187-1204](18/18) | — |
| 83 | Human | 19 | [1179-1197](18/19) | [850-866](17/17) |
| 84 | Human | 19 | — | [847-864](18/18) |
| 85 | Human | 19 | — | — |
| 86 | Human | 19 | — | — |
| 87 | Human | 19 | [1151-1168](17/18) | — |
| 88 | Human | 19 | [1147-1163](17/17) | [818-830](13/13) |
| 89 | Human | 19 | — | [796-813](17/18) |
| 90 | Human | 19 | — | — |
| 91 | Human | 19 | — | — |
| 92 | Human | 19 | — | — |
| 93 | Human | 19 | [1107-1124](17/18) | [777-794](18/18) |
| 94 | Human | 19 | [1107-1117](11/11) | [775-792](18/18) |
| 95 | Human | 19 | [1107-1117](11/11) | [775-789](15/15) |
| 96 | Human | 19 | [1092-1102](11/11) | — |
| 97 | Human | 19 | — | — |
| 98 | Human | 19 | [1068-1079](12/12) | [736-747](12/12) |
| 99 | Human | 19 | — | [730-747](17/18) |
| 100 | Human | 19 | — | [730-746](16/17) |
| 101 | Human | 19 | [1044-1054](11/11) | [715-725](11/11) |
| 102 | Human | 19 | — | — |
| 103 | Human | 19 | — | — |
| 104 | Human | 19 | [1017-1033](16/17) | [686-704](18/19) |
| 105 | Human | 19 | — | — |
| 106 | Human | 19 | [921-934](14/14) | [592-605](14/14) |
| 107 | Human | 19 | [916-934](18/19) | [587-605](18/19) |
| 108 | Human | 19 | [909-927](18/19) | [580-598](18/19) |
| 109 | Human | 19 | — | — |
| 110 | Human | 19 | — | — |
| 111 | Human | 19 | — | — |
| 112 | Human | 19 | — | — |
| 113 | Human | 19 | — | — |
| 114 | Human | 19 | [803-844](15/15) | [501-515](15/15) |
| 115 | Human | 19 | [712-727](15/16) | [383-401](18/19) |
| 116 | Human | 19 | [711-727](16/17) | [382-400](18/19) |

FOR 21
STRETCHES:

| | | | | |
|---|---|---|---|---|
| 1 | Cross Sp | 21 | [535-555](21/21) | [206-226](21/21) |
| 2 | Cross Sp | 21 | [539-559](21/21) | [210-230](21/21) |
| 3 | Cross Sp | 21 | [534-554](21/21) | [205-225](21/21) |
| 4 | Cross Sp | 21 | [537-557](21/21) | [208-228](21/21) |
| 5 | Cross Sp | 21 | [536-556](21/21) | [207-227](21/21) |
| 6 | Cross Sp | 21 | [538-558](21/21) | [209-229](21/21) |
| 7 | Cross Sp | 21 | [771-791](21/21) | [442-462](21/21) |
| 8 | Cross Sp | 21 | [587-607](21/21) | [258-278](21/21) |
| 9 | Human | 21 | — | — |
| 10 | Human | 21 | — | — |
| 11 | Human | 21 | — | — |
| 12 | Human | 21 | [1941-1955](15/15) | — |
| 13 | Human | 21 | [1900-1919](19/20) | — |
| 14 | Human | 21 | [1643-1653](11/11) | [1292-1302](11/11) |
| 15 | Human | 21 | — | — |
| 16 | Human | 21 | — | — |
| 17 | Human | 21 | — | — |
| 18 | Human | 21 | — | — |
| 19 | Human | 21 | [1191-1204](14/14) | — |
| 20 | Human | 21 | [1190-1204](15/15) | — |
| 21 | Human | 21 | [1187-1204](18/18) | — |
| 22 | Human | 21 | — | [847-866](20/20) |
| 23 | Human | 21 | — | [842-859](17/18) |
| 24 | Human | 21 | — | [796-813](17/18) |
| 25 | Human | 21 | — | [784-800](16/17) |
| 26 | Human | 21 | [1107-1117](11/11) | [775-791](17/17) |
| 27 | Human | 21 | [1017-1033](16/17) | [685-704](19/20) |
| 28 | Human | 21 | — | — |
| 29 | Human | 21 | — | — |
| 30 | Human | 21 | [711-727](16/17) | [382-402](20/21) |
| 31 | Human | 21 | [611-627](17/17) | [282-299](18/18) |
| 32 | Human | 21 | [597-617](20/21) | [268-288](21/21) |
| 33 | Human | 21 | — | — |
| 34 | Human | 21 | — | — |
| 35 | Human | 21 | [64-74](11/11) | — |
| 36 | Human | 21 | — | — |
| 37 | Human | 21 | — | — |
| 38 | Human | 21 | — | — |
| 39 | Human | 21 | [1945-1955](11/11) | — |
| 40 | Human | 21 | [1900-1912](13/13) | — |
| 41 | Human | 21 | [1900-1912](13/13) | — |
| 42 | Human | 21 | — | — |
| 43 | Human | 21 | [1852-1871](19/20) | — |
| 44 | Human | 21 | [1764-1775](12/12) | — |
| 45 | Human | 21 | [1761-1774](14/14) | — |
| 46 | Human | 21 | [1745-1756](12/12) | — |
| 47 | Human | 21 | — | — |
| 48 | Human | 21 | — | — |
| 49 | Human | 21 | — | — |
| 50 | Human | 21 | — | — |
| 51 | Human | 21 | [1373-1385](13/13) | [1034-1046](13/13) |
| 52 | Human | 21 | — | — |
| 53 | Human | 21 | — | — |
| 54 | Human | 21 | — | — |
| 55 | Human | 21 | — | — |
| 56 | Human | 21 | [843-853](11/11) | [869-889](20/21) |
| 57 | Human | 21 | [843-853](11/11) | [514-524](11/11) |
| 58 | Human | 21 | [1194-1204](11/11) | [514-524](11/11) |
| 59 | Human | 21 | [1187-1204](18/18) | [855-875](20/21) |
| 60 | Human | 21 | — | [847-864](18/18) |
| 61 | Human | 21 | — | [842-858](16/17) |
| 62 | Human | 21 | — | — |
| 63 | Human | 21 | — | — |
| 64 | Human | 21 | — | — |
| 65 | Human | 21 | [1150-1168](18/19) | — |
| 66 | Human | 21 | [1147-1163](17/17) | [818-830](13/13) |
| 67 | Human | 21 | — | — |
| 68 | Human | 21 | — | — |
| 69 | Human | 21 | — | — |
| 70 | Human | 21 | — | [782-800](18/19) |
| 71 | Human | 21 | — | [780-800](20/21) |
| 72 | Human | 21 | [1107-1123](16/17) | [775-794](20/20) |
| 73 | Human | 21 | [1107-1117](11/11) | [769-789](20/21) |
| 74 | Human | 21 | [1092-1102](11/11) | — |
| 75 | Human | 21 | — | — |
| 76 | Human | 21 | — | — |
| 77 | Human | 21 | — | — |
| 78 | Human | 21 | [1068-1080](13/13) | [736-747](12/12) |
| 79 | Human | 21 | — | — |
| 80 | Human | 21 | [1044-1054](11/11) | [715-725](11/11) |
| 81 | Human | 21 | [1044-1054](11/11) | [715-725](11/11) |
| 82 | Human | 21 | — | — |
| 83 | Human | 21 | — | — |
| 84 | Human | 21 | [1017-1033](16/17) | [688-704](16/17) |
| 85 | Human | 21 | [877-893](16/17) | [547-564](17/18) |
| 86 | Human | 21 | — | — |
| 87 | Human | 21 | — | — |
| 88 | Human | 21 | — | — |
| 89 | Human | 21 | [831-851](20/21) | [502-522](20/21) |
| 90 | Human | 21 | [829-849](20/21) | [500-520](20/21) |
| 91 | Human | 21 | [828-844](17/17) | [499-515](17/17) |
| 92 | Human | 21 | [717-733](16/17) | [388-404](17/17) |
| 93 | Human | 21 | [717-733](16/17) | [388-404](17/17) |
| 94 | Human | 21 | [710-727](17/18) | [381-401](20/21) |
| 95 | Human | 21 | [708-727](19/20) | [379-399](20/21) |
| 96 | Human | 21 | — | — |
| 97 | Human | 21 | — | — |
| 98 | Human | 21 | [596-616](20/21) | [267-287](21/21) |
| 99 | Human | 21 | — | — |
| 100 | Human | 21 | — | — |
| 101 | Human | 21 | — | — |
| 102 | Human | 21 | — | — |
| 103 | Human | 21 | — | — |
| 104 | Human | 21 | [1742-1752](11/11) | — |
| 105 | Human | 21 | — | — |
| 106 | Human | 21 | — | — |
| 107 | Human | 21 | — | — |
| 108 | Human | 21 | [64-74](11/11) | — |

Table I below shows further siRNA compounds specific for the SGPP1 gene that have been selected according to the present invention.

TABLE I

| | | | | | |
|---|---|---|---|---|---|
| | | Oligo | | | |
| No | Source | Length | Sense siRNA | AntiSense siRNA | human 40254975 |

FOR 19
STRETCHES:

| | | | | | |
|---|---|---|---|---|---|
| 1 | Cross Sp | 19 | UGUUUGGAAAAGCCAUAUU | AAUAUGGCUUUUCCAAACA | [1176-1194](19/19) |
| 2 | Cross Sp | 19 | GGACCUGAUUGACAACUUC | GAAGUUGUCAAUCAGGUCC | [928-946](19/19) |
| 3 | Cross Sp | 19 | UCUGGGUGCUGGUCAUGUA | UACAUGACCAGCACCCAGA | [591-609](19/19) |
| 4 | Cross Sp | 19 | UCAUCUGGGUGCUGGUCAU | AUGACCAGCACCCAGAUGA | [588-606](19/19) |
| 5 | Cross Sp | 19 | UGAUUGACAACUUCAACCA | UGGUUGAAGUUGUCAAUCA | [933-951](19/19) |
| 6 | Cross Sp | 19 | ACCUGAUUGACAACUUCAA | UUGAAGUUGUCAAUCAGGU | [930-948](19/19) |
| 7 | Cross Sp | 19 | CUAGUUUGCCUAAGUAGAA | UUCUACUUAGGCAAACUAG | [824-842](19/19) |
| 8 | Cross Sp | 19 | UGACAACUUCAACCAAACU | AGUUUGGUUGAAGUUGUCA | [937-955](19/19) |
| 9 | Cross Sp | 19 | UUGACAACUUCAACCAAAC | GUUUGGUUGAAGUUGUCAA | [936-954](19/19) |
| 10 | Cross Sp | 19 | AUUGACAACUUCAACCAAA | UUUGGUUGAAGUUGUCAAU | [935-953](19/19) |
| 11 | Cross Sp | 19 | CUGAUUGACAACUUCAACC | GGUUGAAGUUGUCAAUCAG | [932-950](19/19) |
| 12 | Cross Sp | 19 | GACCUGAUUGACAACUUCA | UGAAGUUGUCAAUCAGGUC | [929-947](19/19) |
| 13 | Cross Sp | 19 | UGGACCUGAUUGACAACUU | AAGUUGUCAAUCAGGUCCA | [927-945](19/19) |
| 14 | Cross Sp | 19 | GUGGACCUGAUUGACAACU | AGUUGUCAAUCAGGUCCAC | [926-944](19/19) |
| 15 | Cross Sp | 19 | UUCCCCUUCUGGAUCUGGA | UCCAGAUCCAGAAGGGGAA | [536-554](19/19) |
| 16 | Cross Sp | 19 | GAUUGACAACUUCAACCAA | UUGGUUGAAGUUGUCAAUC | [934-952](19/19) |
| 17 | Cross Sp | 19 | UCCCCUUCUGGAUCUGGAA | UUCCAGAUCCAGAAGGGGA | [537-555](19/19) |
| 18 | Cross Sp | 19 | CCUGAUUGACAACUUCAAC | GUUGAAGUUGUCAAUCAGG | [931-949](19/19) |
| 19 | Cross Sp | 19 | UAGUUUGCCUAAGUAGAAU | AUUCUACUUAGGCAAACUA | [825-843](19/19) |
| 20 | Cross Sp | 19 | UCUUCCCCUUCUGGAUCUG | CAGAUCCAGAAGGGGAAGA | [534-552](19/19) |
| 21 | Cross Sp | 19 | UUCUUCCCCUUCUGGAUCU | AGAUCCAGAAGGGGAAGAA | [533-551](19/19) |
| 22 | Cross Sp | 19 | AUCUGGGUGCUGGUCAUGU | ACAUGACCAGCACCCAGAU | [590-608](19/19) |
| 23 | Cross Sp | 19 | GGGUGCUGGUCAUGUACCU | AGGUACAUGACCAGCACCC | [594-612](19/19) |
| 24 | Cross Sp | 19 | CUGGGUGCUGGUCAUGUAC | GUACAUGACCAGCACCCAG | [592-610](19/19) |
| 25 | Cross Sp | 19 | UGGUCAUGUACCUGGGCCA | UGGCCCAGGUACAUGACCA | [600-618](19/19) |
| 26 | Cross Sp | 19 | CAUCUGGGUGCUGGUCAUG | CAUGACCAGCACCCAGAUG | [589-607](19/19) |
| 27 | Cross Sp | 19 | AUGUACCUGGGCCAGUGCA | UGCACUGGCCCAGGUACAU | [605-623](19/19) |
| 28 | Cross Sp | 19 | UCAUGUACCUGGGCCAGUG | CACUGGCCCAGGUACAUGA | [603-621](19/19) |
| 29 | Cross Sp | 19 | UGCUGGUCAUGUACCUGGG | CCCAGGUACAUGACCAGCA | [597-615](19/19) |
| 30 | Cross Sp | 19 | GUGCUGGUCAUGUACCUGG | CCAGGUACAUGACCAGCAC | [596-614](19/19) |
| 31 | Cross Sp | 19 | CUUCCCCUUCUGGAUCUGG | CCAGAUCCAGAAGGGGAAG | [535-553](19/19) |
| 32 | Cross Sp | 19 | GUCAUGUACCUGGGCCAGU | ACUGGCCCAGGUACAUGAC | [602-620](19/19) |
| 33 | Cross Sp | 19 | GGUGCUGGUCAUGUACCUG | CAGGUACAUGACCAGCACC | [595-613](19/19) |
| 34 | Cross Sp | 19 | UGGGUGCUGGUCAUGUACC | GGUACAUGACCAGCACCCA | [593-611](19/19) |
| 35 | Cross Sp | 19 | CUUGACACCUGGAGCACAU | AUGUGCUCCAGGUGUCAAG | [1019-1037](19/19) |
| 36 | Cross Sp | 19 | AGUGCACCAAGGACAUCAU | AUGAUGUCCUUGGUGCACU | [618-636](19/19) |
| 37 | Cross Sp | 19 | CUGUUUGGAAAAGCCAUAU | AUAUGGCUUUUCCAAACAG | [1175-1193](19/19) |
| 38 | Cross Sp | 19 | UCAUCAUCAUCGGGCUUCA | UGAAGCCCGAUGAUGAUGA | [972-990](19/19) |
| 39 | Cross Sp | 19 | GUGCACCAAGGACAUCAUC | GAUGAUGUCCUUGGUGCAC | [619-637](19/19) |
| 40 | Cross Sp | 19 | CAGUGCACCAAGGACAUCA | UGAUGUCCUUGGUGCACUG | [617-635](19/19) |
| 41 | Cross Sp | 19 | UCUGUUUGGAAAAGCCAUA | UAUGGCUUUUCCAAACAGA | [1174-1192](19/19) |
| 42 | Cross Sp | 19 | CUCUGUUUGGAAAAGCCAU | AUGGCUUUUCCAAACAGAG | [1173-1191](19/19) |
| 43 | Cross Sp | 19 | ACUCUGUUUGGAAAAGCCA | UGGCUUUUCCAAACAGAGU | [1172-1190](19/19) |
| 44 | Cross Sp | 19 | UUGACACCUGGAGCACAUC | GAUGUGCUCCAGGUGUCAA | [1020-1038](19/19) |
| 45 | Cross Sp | 19 | UGCACCAAGGACAUCAUCC | GGAUGAUGUCCUUGGUGCA | [620-638](19/19) |
| 46 | Cross Sp | 19 | GCACCAAGGACAUCAUCCG | CGGAUGAUGUCCUUGGUGC | [621-639](19/19) |
| 47 | Cross Sp | 19 | GCCAGUGCACCAAGGACAU | AUGUCCUUGGUGCACUGGC | [615-633](19/19) |
| 48 | Cross Sp | 19 | UGACACCUGGAGCACAUCC | GGAUGUGCUCCAGGUGUCA | [1021-1039](19/19) |
| 49 | Cross Sp | 19 | CCAGUGCACCAAGGACAUC | GAUGUCCUUGGUGCACUGG | [616-634](19/19) |
| 50 | Cross Sp | 19 | AGCACAUCCCGAGGAGACA | UGUCUCCUCGGGAUGUGCU | [1031-1049](19/19) |
| 51 | Human | 19 | GCAUGUGGAUCUCAUGUUA | UAACAUGAGAUCCACAUGC | [1082-1100](19/19) |
| 52 | Human | 19 | CAACCAAACUCACAAAUAU | AUAUUUGUGAGUUUGGUUG | [946-964](19/19) |
| 53 | Human | 19 | GCAGUACCCUCUUAUAUAU | AUAUAUAAGAGGGUACUGC | [775-793](19/19) |
| 54 | Human | 19 | GCAACGAACUCUUCUACAU | AUGUAGAAGAGUUCGUUGC | [510-528](19/19) |
| 55 | Human | 19 | CCAGAACUAUCACUUCAGA | UCUGAAGUGAUAGUUCUGG | [2895-2913](19/19) |
| 56 | Human | 19 | GCACCGUUAACUUUUAAUA | UAUUAAAAGUUAACGGUGC | [2592-2610](19/19) |
| 57 | Human | 19 | GCACAUACUUCAUAUGUAA | UUACAUAUGAAGUAUGUGC | [2532-2550](19/19) |
| 58 | Human | 19 | GGACAAUGCACAUACUUCA | UGAAGUAUGUGCAUUGUCC | [2525-2543](19/19) |
| 59 | Human | 19 | GUAGGUAUUUCCAGUGAAU | AUUCACUGGAAAUACCUAC | [2483-2501](19/19) |
| 60 | Human | 19 | GAACAUGGGAGGCAUUUUU | AAAAAUGCCUCCCAUGUUC | [2009-2027](19/19) |
| 61 | Human | 19 | GCAUAUGCUACUAUACAUU | AAUGUAUAGUAGCAUAUGC | [1937-1955](19/19) |
| 62 | Human | 19 | GGAGCCAUUUCCUAAUUGA | UCAAUUAGGAAAUGGCUCC | [1894-1912](19/19) |
| 63 | Human | 19 | GGAAGUUGAACUUCCUUAU | AUAAGGAAGUUCAACUUCC | [1321-1339](19/19) |
| 64 | Human | 19 | GCACAUGGAAGUUGAACUU | AAGUUCAACUUCCAUGUGC | [1315-1333](19/19) |
| 65 | Human | 19 | CCAUUACUGUGACUCUGUU | AACAGAGUCACAGUAAUGG | [1161-1179](19/19) |
| 66 | Human | 19 | CGAGAUACUAGGAAGUGGU | ACCACUUCCUAGUAUCUCG | [1054-1072](19/19) |
| 67 | Human | 19 | CAUCGGGCUUCAUUUAGCU | AGCUAAAUGAAGCCCGAUG | [979-997](19/19) |
| 68 | Human | 19 | ACAACUUCAACCAAACUCA | UGAGUUUGGUUGAAGUUGU | [939-957](19/19) |
| 69 | Human | 19 | CCAUUUUAAUCUUAGCUGU | ACAGCUAAGAUUAAAAUGG | [894-912](19/19) |
| 70 | Human | 19 | GGAUUCCUAUAUACCAUUU | AAAUGGUAUAUAGGAAUCC | [881-899](19/19) |
| 71 | Human | 19 | GGAUAUUAUUGCUGGAUUC | GAAUCCAGCAAUAAUAUCC | [868-886](19/19) |
| 72 | Human | 19 | GCACUCUAUUCUGGAUAUU | AAUAUCCAGAAUAGAGUGC | [856-874](19/19) |
| 73 | Human | 19 | GGAAUGCACUCUAUUCUGG | CCAGAAUAGAGUGCAUUCC | [851-869](19/19) |
| 74 | Human | 19 | ACAUGGGAAUGCACUCUAU | AUAGAGUGCAUUCCCAUGU | [846-864](19/19) |
| 75 | Human | 19 | GUAGAAUUUACAUGGGAAU | AUUCCCAUGUAAAUUCUAC | [837-855](19/19) |
| 76 | Human | 19 | GUACCCUCUUAUAUAUGGA | UCCAUAUAUAAGAGGGUAC | [778-796](19/19) |
| 77 | Human | 19 | GCUGGCAGUACCCUCUUAU | AUAAGAGGGUACUGCCAGC | [771-789](19/19) |
| 78 | Human | 19 | CCAUUUCUAUGGUCCUCCU | AGGAGGACCAUAGAAAUGG | [741-759](19/19) |
| 79 | Human | 19 | CCAUCCCCAUUUCUAUGGU | ACCAUAGAAAUGGGGAUGG | [735-753](19/19) |
| 80 | Human | 19 | UCAAGUUGGAGGUCUUCUA | UAGAAGACCUCCAACUUGA | [669-687](19/19) |
| 81 | Human | 19 | ACAGUUCUAUGCUAAAUGU | ACAUUUAGCAUAGAACUGU | [3259-3277](19/19) |
| 82 | Human | 19 | CCUAUGCUAUCCUGUUGAU | AUCAACAGGAUAGCAUAGG | [3174-3192](19/19) |
| 83 | Human | 19 | GGACCGUUUCCCAUAAUUU | AAAUUAUGGGAAACGGUCC | [3152-3170](19/19) |
| 84 | Human | 19 | GGGACCGUUUCCCAUAAUU | AAUUAUGGGAAACGGUCCC | [3151-3169](19/19) |
| 85 | Human | 19 | GUACGUUGGAAAAUGUGAA | UUCACAUUUUCCAACGUAC | [3088-3106](19/19) |
| 86 | Human | 19 | GGAAAAUAUUGGUACGUUG | CAACGUACCAAUAUUUUCC | [3077-3095](19/19) |
| 87 | Human | 19 | GAAGCUGGUAAUUUUAAGU | ACUUAAAAUUACCAGCUUC | [3004-3022](19/19) |
| 88 | Human | 19 | CAAAACUGAAGCUGGUAAU | AUUACCAGCUUCAGUUUUG | [2997-3015](19/19) |
| 89 | Human | 19 | CCAAAACUGAAGCUGGUAA | UUACCAGCUUCAGUUUUGG | [2996-3014](19/19) |
| 90 | Human | 19 | GCAUGGUAAUUUUUACCAA | UUGGUAAAAAUUACCAUGC | [2981-2999](19/19) |
| 91 | Human | 19 | UUAGGGGCAUGGUAAUUUU | AAAAUUACCAUGCCCCUAA | [2975-2993](19/19) |
| 92 | Human | 19 | CACUUCAGAUGUUUCUAUU | AAUAGAAACAUCUGAAGUG | [2905-2923](19/19) |
| 93 | Human | 19 | UGAUUAUCCAGAACUAUCA | UGAUAGUUCUGGAUAAUCA | [2888-2906](19/19) |
| 94 | Human | 19 | GUAUGAUUAUCCAGAACUA | UAGUUCUGGAUAAUCAUAC | [2885-2903](19/19) |
| 95 | Human | 19 | CCAGGUAUGAAAUUACUGA | UCAGUAAUUUCAUACCUGG | [2850-2868](19/19) |
| 96 | Human | 19 | GAAGAGAUUAACUUUCCAU | AUGGAAAGUUAAUCUCUUC | [2820-2838](19/19) |
| 97 | Human | 19 | GGAAAAUUUGCGUAUAGUA | UACUAUACGCAAAUUUUCC | [2708-2726](19/19) |
| 98 | Human | 19 | CCCAUUUAUUGGCUAUUUA | UAAAUAGCCAAUAAAUGGG | [2611-2629](19/19) |
| 99 | Human | 19 | GGAUUUAGCACCGUUAACU | AGUUAACGGUGCUAAAUCC | [2585-2603](19/19) |
| 100 | Human | 19 | GGAAAGGAUUGGAUUUAGC | GCUAAAUCCAAUCCUUUCC | [2575-2593](19/19) |
| 101 | Human | 19 | UGAAUGGAAACGUUGAAAU | AUUUCAACGUUUCCAUUCA | [2497-2515](19/19) |
| 102 | Human | 19 | GCAGGACAUAUUUUUUGUA | UACAAAAAAUAUGUCCUGC | [2438-2456](19/19) |
| 103 | Human | 19 | GCUAAACUUUCUCACCAAU | AUUGGUGAGAAAGUUUAGC | [2348-2366](19/19) |
| 104 | Human | 19 | GGAAUGGGCUAAACUUUCU | AGAAAGUUUAGCCCAUUCC | [2341-2359](19/19) |
| 105 | Human | 19 | CAGGGUGACAGGCAUAAAA | UUUUAUGCCUGUCACCCUG | [2278-2296](19/19) |
| 106 | Human | 19 | CCUUUCCGUUGCUAAAUUA | UAAUUUAGCAACGGAAAGG | [2168-2186](19/19) |
| 107 | Human | 19 | CUGGAUGUCAGUUUAAUAA | UUAUUAAACUGACAUCCAG | [2115-2133](19/19) |
| 108 | Human | 19 | GAACUUUAUUUGCAGCUAU | AUAGCUGCAAAUAAAGUUC | [2095-2113](19/19) |
| 109 | Human | 19 | CCAUUACCUUUCAUGUUAU | AUAACAUGAAAGGUAAUGG | [1980-1998](19/19) |
| 110 | Human | 19 | GAAUGCAUAUGCUACUAUA | UAUAGUAGCAUAUGCAUUC | [1933-1951](19/19) |
| 111 | Human | 19 | AGACCAGAAUGCAUAUGCU | AGCAUAUGCAUUCUGGUCU | [1927-1945](19/19) |
| 112 | Human | 19 | GUAAUUACUAGACCAGAAU | AUUCUGGUCUAGUAAUUAC | [1918-1936](19/19) |
| 113 | Human | 19 | CUACAUACAGGUAUGUUGU | ACAACAUACCUGUAUGUAG | [1841-1859](19/19) |
| 114 | Human | 19 | GAACUACAUACAGGUAUGU | ACAUACCUGUAUGUAGUUC | [1838-1856](19/19) |
| 115 | Human | 19 | CUGUGUAUUUGAACUACAU | AUGUAGUUCAAAUACACAG | [1828-1846](19/19) |
| 116 | Human | 19 | AAACAGUUGUGCCUGUGUA | UACACAGGCACAACUGUUU | [1816-1834](19/19) |
| 117 | Human | 19 | CUAUGGGAGUGUAUUAUAA | UUAUAAUACACUCCCAUAG | [1780-1798](19/19) |
| 118 | Human | 19 | GGAGAGUUUUGUAUUAUGG | CCAUAAUACAAAACUCUCC | [1664-1682](19/19) |
| 119 | Human | 19 | CCAUUUAGAAUGUUCAUGU | ACAUGAACAUUCUAAAUGG | [1638-1656](19/19) |
| 120 | Human | 19 | UGAGCUAUUUCGUUACUGA | UCAGUAACGAAAUAGCUCA | [1604-1622](19/19) |
| 121 | Human | 19 | GGCUUUUGCAGGAAUUUAA | UUAAAUUCCUGCAAAAGCC | [1507-1525](19/19) |
| 122 | Human | 19 | UUAGGCUUUUGCAGGAAUU | AAUUCCUGCAAAAGCCUAA | [1504-1522](19/19) |
| 123 | Human | 19 | GGUAAAGCCAGGUCAGAAU | AUUCUGACCUGGCUUUACC | [1486-1504](19/19) |
| 124 | Human | 19 | GAGGGUAUCAGUUACUGAU | AUCAGUAACUGAUACCCUC | [1449-1467](19/19) |
| 125 | Human | 19 | GGAGAAGUAUUGUUUAUGA | UCAUAAACAAUACUUCUCC | [1422-1440](19/19) |
| 126 | Human | 19 | GUUGGUUUCUCCAUCACAU | AUGUGAUGGAGAAACCAAC | [1361-1379](19/19) |
| 127 | Human | 19 | CCUAUGGAAUGGUUGGUUU | AAACCAACCAUUCCAUAGG | [1350-1368](19/19) |
| 128 | Human | 19 | CGGUAUAUUACCUAUGGAA | UUCCAUAGGUAAUAUACCG | [1340-1358](19/19) |
| 129 | Human | 19 | GAACUUCCUUAUCGGUAUA | UAUACCGAUAAGGAAGUUC | [1328-1346](19/19) |
| 130 | Human | 19 | GACAGCACAUGGAAGUUGA | UCAACUUCCAUGUGCUGUC | [1311-1329](19/19) |
| 131 | Human | 19 | CAAGACAGCACAUGGAAGU | ACUUCCAUGUGCUGUCUUG | [1308-1326](19/19) |
| 132 | Human | 19 | CGAAAAGCAAGACAGCACA | UGUGCUGUCUUGCUUUUCG | [1301-1319](19/19) |
| 133 | Human | 19 | UGAUGAUAUUCGAAAAGCA | UGCUUUUCGAAUAUCAUCA | [1291-1309](19/19) |
| 134 | Human | 19 | GCAAAAUCUUCAAUAUACC | GGUAUAUUGAAGAUUUUGC | [1269-1287](19/19) |
| 135 | Human | 19 | CCUGCAAAAUCUUCAAUAU | AUAUUGAAGAUUUUGCAGG | [1266-1284](19/19) |
| 136 | Human | 19 | CCUUUAGCCUGCAAAAUCU | AGAUUUUGCAGGCUAAAGG | [1259-1277](19/19) |
| 137 | Human | 19 | GGAUGGUAUUUGUACUAAU | AUUAGUACAAAUACCAUCC | [1209-1227](19/19) |
| 138 | Human | 19 | UCAUAGGGAUGGUAUUUGU | ACAAAUACCAUCCCUAUGA | [1203-1221](19/19) |
| 139 | Human | 19 | CUGUGACUCUGUUUGGAAA | UUUCCAAACAGAGUCACAG | [1167-1185](19/19) |
| 140 | Human | 19 | AGAUACAUUACCUUUAGCU | AGCUAAAGGUAAUGUAUCU | [1135-1153](19/19) |
| 141 | Human | 19 | AGAUCCUUCUCUAGAUACA | UGUAUCUAGAGAAGGAUCU | [1123-1141](19/19) |
| 142 | Human | 19 | UUAGAUCCUUCUCUAGAUA | UAUCUAGAGAAGGAUCUAA | [1121-1139](19/19) |
| 143 | Human | 19 | CAUGGGUCUAGUAUUAGAU | AUCUAAUACUAGACCCAUG | [1108-1126](19/19) |
| 144 | Human | 19 | UAACAUGGGUCUAGUAUUA | UAAUACUAGACCCAUGUUA | [1105-1123](19/19) |
| 145 | Human | 19 | GGAUCUCAUGUUACUUAUA | UAUAAGUAACAUGAGAUCC | [1088-1106](19/19) |
| 146 | Human | 19 | UGGAUCUCAUGUUACUUAU | AUAAGUAACAUGAGAUCCA | [1087-1105](19/19) |
| 147 | Human | 19 | GUGGAUCUCAUGUUACUUA | UAAGUAACAUGAGAUCCAC | [1086-1104](19/19) |
| 148 | Human | 19 | AUGUGGAUCUCAUGUUACU | AGUAACAUGAGAUCCACAU | [1084-1102](19/19) |
| 149 | Human | 19 | GGAAUUGCAUGUGGAUCUC | GAGAUCCACAUGCAAUUCC | [1076-1094](19/19) |

FOR 21
STRETCHES:

| | | | | | |
|---|---|---|---|---|---|
| 1 | Cross Sp | 21 | CUGAUUGACAACUUCAACCAA | UUGGUUGAAGUUGUCAAUCAG | [932-952](21/21) |
| 2 | Cross Sp | 21 | AUUGACAACUUCAACCAAACU | AGUUUGGUUGAAGUUGUCAAU | [935-955](21/21) |
| 3 | Cross Sp | 21 | GACCUGAUUGACAACUUCAAC | GUUGAAGUUGUCAAUCAGGUC | [929-949](21/21) |
| 4 | Cross Sp | 21 | GGACCUGAUUGACAACUUCAA | UUGAAGUUGUCAAUCAGGUCC | [928-948](21/21) |
| 5 | Cross Sp | 21 | UGGACCUGAUUGACAACUUCA | UGAAGUUGUCAAUCAGGUCCA | [927-947](21/21) |
| 6 | Cross Sp | 21 | CUUCCCCUUCUGGAUCUGGAA | UUCCAGAUCCAGAAGGGGAAG | [535-555](21/21) |
| 7 | Cross Sp | 21 | AUCUGGGUGCUGGUCAUGUAC | GUACAUGACCAGCACCCAGAU | [590-610](21/21) |
| 8 | Cross Sp | 21 | UGAUUGACAACUUCAACCAAA | UUUGGUUGAAGUUGUCAAUCA | [933-953](21/21) |
| 9 | Cross Sp | 21 | CCUGAUUGACAACUUCAACCA | UGGUUGAAGUUGUCAAUCAGG | [931-951](21/21) |
| 10 | Cross Sp | 21 | UCAUCUGGGUGCUGGUCAUGU | ACAUGACCAGCACCCAGAUGA | [588-608](21/21) |
| 11 | Cross Sp | 21 | GUGGACCUGAUUGACAACUUC | GAAGUUGUCAAUCAGGUCCAC | [926-946](21/21) |
| 12 | Cross Sp | 21 | CAUCUGGGUGCUGGUCAUGUA | UACAUGACCAGCACCCAGAUG | [589-609](21/21) |
| 13 | Cross Sp | 21 | GAUUGACAACUUCAACCAAAC | GUUUGGUUGAAGUUGUCAAUC | [934-954](21/21) |
| 14 | Cross Sp | 21 | UUCUUCCCCUUCUGGAUCUGG | CCAGAUCCAGAAGGGGAAGAA | [533-553](21/21) |
| 15 | Cross Sp | 21 | ACCUGAUUGACAACUUCAACC | GGUUGAAGUUGUCAAUCAGGU | [930-950](21/21) |
| 16 | Cross Sp | 21 | UCUUCCCCUUCUGGAUCUGGA | UCCAGAUCCAGAAGGGGAAGA | [534-554](21/21) |
| 17 | Cross Sp | 21 | UGGGUGCUGGUCAUGUACCUG | CAGGUACAUGACCAGCACCCA | [593-613](21/21) |
| 18 | Cross Sp | 21 | UCAUGUACCUGGGCCAGUGCA | UGCACUGGCCCAGGUACAUGA | [603-623](21/21) |
| 19 | Cross Sp | 21 | CUGGGUGCUGGUCAUGUACCU | AGGUACAUGACCAGCACCCAG | [592-612](21/21) |
| 20 | Cross Sp | 21 | UCUGGGUGCUGGUCAUGUACC | GGUACAUGACCAGCACCCAGA | [591-611](21/21) |
| 21 | Cross Sp | 21 | UGGUCAUGUACCUGGGCCAGU | ACUGGCCCAGGUACAUGACCA | [600-620](21/21) |
| 22 | Cross Sp | 21 | CUCUGUUUGGAAAAGCCAUAU | AUAUGGCUUUUCCAAACAGAG | [1173-1193](21/21) |
| 23 | Cross Sp | 21 | CAGUGCACCAAGGACAUCAUC | GAUGAUGUCCUUGGUGCACUG | [617-637](21/21) |
| 24 | Cross Sp | 21 | CCAGUGCACCAAGGACAUCAU | AUGAUGUCCUUGGUGCACUGG | [616-636](21/21) |
| 25 | Cross Sp | 21 | UCUGUUUGGAAAAGCCAUAUU | AAUAUGGCUUUUCCAAACAGA | [1174-1194](21/21) |
| 26 | Cross Sp | 21 | AGUGCACCAAGGACAUCAUCC | GGAUGAUGUCCUUGGUGCACU | [618-638](21/21) |
| 27 | Cross Sp | 21 | ACUCUGUUUGGAAAAGCCAUA | UAUGGCUUUUCCAAACAGAGU | [1172-1192](21/21) |
| 28 | Cross Sp | 21 | CUUGACACCUGGAGCACAUCC | GGAUGUGCUCCAGGUGUCAAG | [1019-1039](21/21) |
| 29 | Cross Sp | 21 | GCCAGUGCACCAAGGACAUCA | UGAUGUCCUUGGUGCACUGGC | [615-635](21/21) |
| 30 | Cross Sp | 21 | GUGCACCAAGGACAUCAUCCG | CGGAUGAUGUCCUUGGUGCAC | [619-639](21/21) |
| 31 | Cross Sp | 21 | UUGACACCUGGAGCACAUCCC | GGGAUGUGCUCCAGGUGUCAA | [1020-1040](21/21) |
| 32 | Human | 21 | GAAGUGGUGCUGGAAUUGCAU | AUGCAAUUCCAGCACCACUUC | [1065-1085](21/21) |
| 33 | Human | 21 | CAAGGCAUGUAGGAAAAUAUU | AAUAUUUUCCUACAUGCCUUG | [3066-3086](21/21) |
| 34 | Human | 21 | CCAAAACUGAAGCUGGUAAUU | AAUUACCAGCUUCAGUUUUGG | [2996-3016](21/21) |
| 35 | Human | 21 | GAAGUGUGGUUGUACUUACGU | ACGUAAGUACAACCACACUUC | [2763-2783](21/21) |
| 36 | Human | 21 | GGAGGGUAUCAGUUACUGAUA | UAUCAGUAACUGAUACCCUCC | [1448-1468](21/21) |
| 37 | Human | 21 | GCAAGACAGCACAUGGAAGUU | AACUUCCAUGUGCUGUCUUGC | [1307-1327](21/21) |
| 38 | Human | 21 | CCAUUCCUUUAGCCUGCAAAA | UUUUGCAGGCUAAAGGAAUGG | [1254-1274](21/21) |
| 39 | Human | 21 | GUGGAUCUCAUGUUACUUAUA | UAUAAGUAACAUGAGAUCCAC | [1086-1106](21/21) |
| 40 | Human | 21 | CAUGUGGAUCUCAUGUUACUU | AAGUAACAUGAGAUCCACAUG | [1083-1103](21/21) |
| 41 | Human | 21 | GGAGACACAGCCGAGAUACUA | UAGUAUCUCGGCUGUGUCUCC | [1043-1063](21/21) |
| 42 | Human | 21 | UCAUCGGGCUUCAUUUAGCUU | AAGCUAAAUGAAGCCCGAUGA | [978-998](21/21) |
| 43 | Human | 21 | CCAUUCAUCAUCAUCGGGCUU | AAGCCCGAUGAUGAUGAAUGG | [968-988](21/21) |
| 44 | Human | 21 | ACAAAUAUGCUCCAUUCAUCA | UGAUGAAUGGAGCAUAUUUGU | [957-977](21/21) |
| 45 | Human | 21 | CUCACAAAUAUGCUCCAUUCA | UGAAUGGAGCAUAUUUGUGAG | [954-974](21/21) |
| 46 | Human | 21 | CUUCAACCAAACUCACAAAUA | UAUUUGUGAGUUUGGUUGAAG | [943-963](21/21) |
| 47 | Human | 21 | CCAUUUGUGGACCUGAUUGAC | GUCAAUCAGGUCCACAAAUGG | [920-940](21/21) |
| 48 | Human | 21 | UUAGCUGUCUUCUAUCCAUUU | AAAUGGAUAGAAGACAGCUAA | [905-925](21/21) |
| 49 | Human | 21 | CGCUGGCAGUACCCUCUUAUA | UAUAAGAGGGUACUGCCAGCG | [770-790](21/21) |
| 50 | Human | 21 | GAACUCUUCUACAUCCUGUUC | GAACAGGAGGUAGAAGAGUUC | [515-535](21/21) |
| 51 | Human | 21 | GACAGUUCUAUGCUAAAUGUA | UACAUUUAGCAUAGAACUGUC | [3258-3278](21/21) |
| 52 | Human | 21 | CUAUGACAGUUCUAUGCUAAA | UUUAGCAUAGAACUGUCAUAG | [3254-3274](21/21) |
| 53 | Human | 21 | GUCCUAUGACAGUUCUAUGCU | AGCAUAGAACUGUCAUAGGAC | [3251-3271](21/21) |
| 54 | Human | 21 | GCCUAUGCUAUCCUGUUGAUU | AAUCAACAGGAUAGCAUAGGC | [3173-3193](21/21) |
| 55 | Human | 21 | CCAUAAUUUGGGCCUAUGCUA | UAGCAUAGGCCCAAAUUAUGG | [3162-3182](21/21) |
| 56 | Human | 21 | GGAAAAUGUGAAAAGAGCUUU | AAAGCUCUUUUCACAUUUUCC | [3095-3115](21/21) |
| 57 | Human | 21 | GUACGUUGGAAAAUGUGAAAA | UUUUCACAUUUUCCAACGUAC | [3088-3108](21/21) |
| 58 | Human | 21 | GCAUGUAGGAAAAUAUUGGUA | UACCAAUAUUUUCCUACAUGC | [3070-3090](21/21) |
| 59 | Human | 21 | GGAAACCAAGGCAUGUAGGAA | UUCCUACAUGCCUUGGUUUCC | [3060-3080](21/21) |
| 60 | Human | 21 | CCAUAACUAUUUGAGCUCAUA | UAUGAGCUCAAAUAGUUAUGG | [2931-2951](21/21) |
| 61 | Human | 21 | CUAUCACUUCAGAUGUUUCUA | UAGAAACAUCUGAAGUGAUAG | [2901-2921](21/21) |
| 62 | Human | 21 | GAACUAUCACUUCAGAUGUUU | AAACAUCUGAAGUGAUAGUUC | [2898-2918](21/21) |
| 63 | Human | 21 | CAGAACUAUCACUUCAGAUGU | ACAUCUGAAGUGAUAGUUCUG | [2896-2916](21/21) |
| 64 | Human | 21 | CCAGGUAUGAAAUUACUGAUU | AAUCAGUAAUUUCAUACCUGG | [2850-2870](21/21) |
| 65 | Human | 21 | GGUUGUACUUACGUUAAUAGU | ACUAUUAACGUAAGUACAACC | [2770-2790](21/21) |
| 66 | Human | 21 | GUGUGGUUGUACUUACGUUAA | UUAACGUAAGUACAACCACAC | [2766-2786](21/21) |
| 67 | Human | 21 | GGAUUUAGCACCGUUAACUUU | AAAGUUAACGGUGCUAAAUCC | [2585-2605](21/21) |
| 68 | Human | 21 | CAAUGCACAUACUUCAUAUGU | ACAUAUGAAGUAUGUGCAUUG | [2528-2548](21/21) |
| 69 | Human | 21 | GGGACAAUGCACAUACUUCAU | AUGAAGUAUGUGCAUUGUCCC | [2524-2544](21/21) |
| 70 | Human | 21 | GAUGGGACAAUGCACAUACUU | AAGUAUGUGCAUUGUCCCAUC | [2521-2541](21/21) |
| 71 | Human | 21 | GUGAAUGGAAACGUUGAAAUU | AAUUUCAACGUUUCCAUUCAC | [2496-2516](21/21) |
| 72 | Human | 21 | GCAGGACAUAUUUUUUGUACA | UGUACAAAAAAUAUGUCCUGC | [2438-2458](21/21) |
| 73 | Human | 21 | GGAGCAGGACAUAUUUUUUGU | ACAAAAAAUAUGUCCUGCUCC | [2435-2455](21/21) |
| 74 | Human | 21 | GGCUAAACUUUCUCACCAAUU | AAUUGGUGAGAAAGUUUAGCC | [2347-2367](21/21) |
| 75 | Human | 21 | GGAAUGGGCUAAACUUUCUCA | UGAGAAAGUUUAGCCCAUUCC | [2341-2361](21/21) |
| 76 | Human | 21 | GCCUUUCCGUUGCUAAAUUAA | UUAAUUUAGCAACGGAAAGGC | [2167-2187](21/21) |
| 77 | Human | 21 | CUAUUCUGGAUGUCAGUUUAA | UUAAACUGACAUCCAGAAUAG | [2110-2130](21/21) |
| 78 | Human | 21 | GCAGCUAUUCUGGAUGUCAGU | ACUGACAUCCAGAAUAGCUGC | [2106-2126](21/21) |
| 79 | Human | 21 | GAACUUUAUUUGCAGCUAUUC | GAAUAGCUGCAAAUAAAGGUC | [2095-2115](21/21) |
| 80 | Human | 21 | GAACAUGGGAGGCAUUUUUAA | UUAAAAAUGCCUCCCAUGUUC | [2009-2029](21/21) |
| 81 | Human | 21 | UGAACAUGGGAGGCAUUUUUA | UAAAAAUGCCUCCCAUGUUCA | [2008-2028](21/21) |
| 82 | Human | 21 | CCAUUACCUUUCAUGUUAUCC | GGAUAACAUGAAAGGUAAUGG | [1980-2000](21/21) |
| 83 | Human | 21 | CACUGCCAUUACCUUUCAUGU | ACAUGAAAGGUAAUGGCAGUG | [1975-1995](21/21) |
| 84 | Human | 21 | CUAGACCAGAAUGCAUAUGCU | AGCAUAUGCAUUCUGGUCUAG | [1925-1945](21/21) |
| 85 | Human | 21 | UUACUAGACCAGAAUGCAUAU | AUAUGCAUUCUGGUCUAGUAA | [1922-1942](21/21) |
| 86 | Human | 21 | GGAGCCAUUUCCUAAUUGAAU | AUUCAAUUAGGAAAUGGCUCC | [1894-1914](21/21) |
| 87 | Human | 21 | GGGAGCCAUUUCCUAAUUGAA | UUCAAUUAGGAAAUGGCUCCC | [1893-1913](21/21) |
| 88 | Human | 21 | UUACUAUGGGAGCCAUUUCCU | AGGAAAUGGCUCCCAUAGUAA | [1886-1906](21/21) |
| 89 | Human | 21 | CCUGUGUAUUUGAACUACAUA | UAUGUAGUUCAAAUACACAGG | [1827-1847](21/21) |
| 90 | Human | 21 | CAAACAGUUGUGCCUGUGUAU | AUACACAGGCACAACUGUUUG | [1815-1835](21/21) |
| 91 | Human | 21 | GCACAUCCAGAUACUGUUUUA | UAAAACAGUAUCUGGAUGUGC | [1576-1596](21/21) |
| 92 | Human | 21 | GGUCAGAAUUAGGCUUUUGCA | UGCAAAAGCCUAAUUCUGACC | [1496-1516](21/21) |
| 93 | Human | 21 | CCAGGUCAGAAUUAGGCUUUU | AAAAGCCUAAUUCUGACCUGG | [1493-1513](21/21) |
| 94 | Human | 21 | CCAUCACAUUUUUUGUUCCUU | AAGGAACAAAAAAUGUGAUGG | [1371-1391](21/21) |
| 95 | Human | 21 | GGUUUCUCCAUCACAUUUUUU | AAAAAAUGUGAUGGAGAAACC | [1364-1384](21/21) |
| 96 | Human | 21 | GGUUGGUUUCUCCAUCACAUU | AAUGUGAUGGAGAAACCAACC | [1360-1380](21/21) |
| 97 | Human | 21 | ACAUGGAAGUUGAACUUCCUU | AAGGAAGUUCAACUUCCAUGU | [1317-1337](21/21) |
| 98 | Human | 21 | AGACAGCACAUGGAAGUUGAA | UUCAACUUCCAUGUGCUGUCU | [1310-1330](21/21) |
| 99 | Human | 21 | AAAGCAAGACAGCACAUGGAA | UUCCAUGUGCUGUCUUGCUUU | [1304-1324](21/21) |
| 100 | Human | 21 | GUGAUGAUAUUCGAAAAGCAA | UUGCUUUUCGAAUAUCAUCAC | [1290-1310](21/21) |
| 101 | Human | 21 | GCCUGCAAAAUCUUCAAUAUA | UAUAUUGAAGAUUUUGCAGGC | [1265-1285](21/21) |
| 102 | Human | 21 | GAAAAAGAUCACCAUUCCUUU | AAAGGAAUGGUGAUCUUUUUC | [1243-1263](21/21) |
| 103 | Human | 21 | CUCAUAGGGAUGGUAUUUGUA | UACAAAUACCAUCCCUAUGAG | [1202-1222](21/21) |
| 104 | Human | 21 | GGAUCCUCAUAGGGAUGGUAU | AUACCAUCCCUAUGAGGAUCC | [1197-1217](21/21) |
| 105 | Human | 21 | CCCAUUACUGUGACUCUGUUU | AAACAGAGUCACAGUAAUGGG | [1160-1180](21/21) |
| 106 | Human | 21 | CUAGAUACAUUACCUUUAGCU | AGCUAAAGGUAAUGUAUCUAG | [1133-1153](21/21) |
| 107 | Human | 21 | CUAGUAUUAGAUCCUUCUCUA | UAGAGAAGGAUCUAAUACUAG | [1115-1135](21/21) |
| 108 | Human | 21 | GGAUCUCAUGUUACUUAUAAC | GUUAUAAGUAACAUGAGAUCC | [1088-1108](21/21) |
| 109 | Human | 21 | GCAUGUGGAUCUCAUGUUACU | AGUAACAUGAGAUCCACAUGC | [1082-1102](21/21) |
| 110 | Human | 21 | GAAUUGCAUGUGGAUCUCAUG | CAUGAGAUCCACAUGCAAUUC | [1077-1097](21/21) |
| 111 | Human | 21 | GUGCUGGAAUUGCAUGUGGAU | AUCCACAUGCAAUUCCAGCAC | [1071-1091](21/21) |
| 112 | Human | 21 | CUAGGAAGUGGUGCUGGAAUU | AAUUCCAGCACCACUUCCUAG | [1061-1081](21/21) |
| 113 | Human | 21 | CAGCCGAGAUACUAGGAAGUG | CACUUCCUAGUAUCUCGGCUG | [1050-1070](21/21) |
| 114 | Human | 21 | GACACAGCCGAGAUACUAGGA | UCCUAGUAUCUCGGCUGUGUC | [1046-1066](21/21) |
| 115 | Human | 21 | GAUCUUUUCUUUCACUCUUGA | UCAAGAGUGAAAGAAAAGAUC | [1003-1023](21/21) |
| 116 | Human | 21 | GGAUCUUUUCUUUCACUCUUG | CAAGAGUGAAAGAAAAGAUCC | [1002-1022](21/21) |
| 117 | Human | 21 | GGGGAUCUUUUCUUUCACUCU | AGAGUGAAAGAAAAGAUCCCC | [1000-1020](21/21) |
| 118 | Human | 21 | GCUUUGGGGAUCUUUUCUUUC | GAAAGAAAAGAUCCCCAAAGC | [995-1015](21/21) |
| 119 | Human | 21 | UUAGCUUUGGGGAUCUUUUCU | AGAAAAGAUCCCCAAAGCUAA | [992-1012](21/21) |
| 120 | Human | 21 | GGCUUCAUUUAGCUUUGGGGA | UCCCCAAAGCUAAAUGAAGCC | [984-1004](21/21) |
| 121 | Human | 21 | CAUCAUCAUCGGGCUUCAUUU | AAAUGAAGCCCGAUGAUGAUG | [973-993](21/21) |
| 122 | Human | 21 | CAAAUAUGGCUCCAUUCAUCAU | AUGAUGAAUGGAGCAUAUUUG | [958-978](21/21) |
| 123 | Human | 21 | UCACAAAUAUGCUCCAUUCAU | AUGAAUGGAGCAUAUUUGUGA | [955-975](21/21) |
| 124 | Human | 21 | AACCAAACUCACAAAUAUGCU | AGCAUAUUUGUGAGUUUGGUU | [947-967](21/21) |
| 125 | Human | 21 | UCAACCAAACUCACAAAUAUG | CAUAUUUGUGAGUUUGGUUGA | [945-965](21/21) |
| 126 | Human | 21 | CAACUUCAACCAAACUCACAA | UUGUGAGUUUGGUUGAAGUUG | [940-960](21/21) |
| 127 | Human | 21 | GACAACUUCAACCAAACUCAC | GUGAGUUUGGUUGAAGUUGUC | [938-958](21/21) |
| 128 | Human | 21 | UGACAACUUCAACCAAACUCA | UGAGUUUGGUUGAAGUUGUCA | [937-957](21/21) |

| | | | | |
|---|---|---|---|---|
| | | Oligo | | |
| No | Source | Length | mouse 31543695 | rat 34865465 |

FOR 19
STRETCHES:

| | | | | |
|---|---|---|---|---|
| 1 | Cross Sp | 19 | [1168-1186](19/19) | [1487-1505](19/19) |
| 2 | Cross Sp | 19 | [920-938](19/19) | [1239-1257](19/19) |
| 3 | Cross Sp | 19 | [583-601](19/19) | [902-920](19/19) |
| 4 | Cross Sp | 19 | [580-598](19/19) | [899-917](19/19) |
| 5 | Cross Sp | 19 | [925-943](19/19) | [1244-1262](19/19) |
| 6 | Cross Sp | 19 | [922-940](19/19) | [1241-1259](19/19) |
| 7 | Cross Sp | 19 | [816-834](19/19) | [1135-1153](19/19) |
| 8 | Cross Sp | 19 | [929-947](19/19) | [1248-1266](19/19) |
| 9 | Cross Sp | 19 | [928-946](19/19) | [1247-1265](19/19) |
| 10 | Cross Sp | 19 | [927-945](19/19) | [1246-1264](19/19) |
| 11 | Cross Sp | 19 | [924-942](19/19) | [1243-1261](19/19) |
| 12 | Cross Sp | 19 | [921-939](19/19) | [1240-1258](19/19) |
| 13 | Cross Sp | 19 | [919-937](19/19) | [1238-1256](19/19) |
| 14 | Cross Sp | 19 | [918-936](19/19) | [1237-1255](19/19) |
| 15 | Cross Sp | 19 | [528-546](19/19) | [847-865](19/19) |
| 16 | Cross Sp | 19 | [926-944](19/19) | [1245-1263](19/19) |
| 17 | Cross Sp | 19 | [529-547](19/19) | [848-866](19/19) |
| 18 | Cross Sp | 19 | [923-941](19/19) | [1242-1260](19/19) |
| 19 | Cross Sp | 19 | [817-835](19/19) | [1136-1154](19/19) |
| 20 | Cross Sp | 19 | [526-544](19/19) | [845-863](19/19) |
| 21 | Cross Sp | 19 | [525-543](19/19) | [844-862](19/19) |
| 22 | Cross Sp | 19 | [582-600](19/19) | [901-919](19/19) |
| 23 | Cross Sp | 19 | [586-604](19/19) | [905-923](19/19) |
| 24 | Cross Sp | 19 | [584-602](19/19) | [903-921](19/19) |
| 25 | Cross Sp | 19 | [592-610](19/19) | [911-929](19/19) |
| 26 | Cross Sp | 19 | [581-599](19/19) | [900-918](19/19) |
| 27 | Cross Sp | 19 | [597-615](19/19) | [916-934](19/19) |
| 28 | Cross Sp | 19 | [595-613](19/19) | [914-932](19/19) |
| 29 | Cross Sp | 19 | [589-607](19/19) | [908-926](19/19) |
| 30 | Cross Sp | 19 | [588-606](19/19) | [907-925](19/19) |
| 31 | Cross Sp | 19 | [527-545](19/19) | [846-864](19/19) |
| 32 | Cross Sp | 19 | [594-612](19/19) | [913-931](19/19) |
| 33 | Cross Sp | 19 | [587-605](19/19) | [906-924](19/19) |
| 34 | Cross Sp | 19 | [585-603](19/19) | [904-922](19/19) |
| 35 | Cross Sp | 19 | [1011-1029](19/19) | [1330-1348](19/19) |
| 36 | Cross Sp | 19 | [610-628](19/19) | [929-947](19/19) |
| 37 | Cross Sp | 19 | [1167-1185](19/19) | [1486-1504](19/19) |
| 38 | Cross Sp | 19 | [964-982](19/19) | [1283-1301](19/19) |
| 39 | Cross Sp | 19 | [611-629](19/19) | [930-948](19/19) |
| 40 | Cross Sp | 19 | [609-627](19/19) | [928-946](19/19) |
| 41 | Cross Sp | 19 | [1166-1184](19/19) | [1485-1503](19/19) |
| 42 | Cross Sp | 19 | [1165-1183](19/19) | [1484-1502](19/19) |
| 43 | Cross Sp | 19 | [1164-1182](19/19) | [1483-1501](19/19) |
| 44 | Cross Sp | 19 | [1012-1030](19/19) | [1331-1349](19/19) |
| 45 | Cross Sp | 19 | [612-630](19/19) | [931-949](19/19) |
| 46 | Cross Sp | 19 | [613-631](19/19) | [932-950](19/19) |
| 47 | Cross Sp | 19 | [607-625](19/19) | [926-944](19/19) |
| 48 | Cross Sp | 19 | [1013-1031](19/19) | [1332-1350](19/19) |
| 49 | Cross Sp | 19 | [608-626](19/19) | [927-945](19/19) |
| 50 | Cross Sp | 19 | [1023-1041](19/19) | [1342-1360](19/19) |
| 51 | Human | 19 | — | — |
| 52 | Human | 19 | — | — |
| 53 | Human | 19 | — | [1086-1104](18/19) |
| 54 | Human | 19 | [502-520](18/19) | [821-839](18/19) |
| 55 | Human | 19 | — | — |
| 56 | Human | 19 | — | — |
| 57 | Human | 19 | — | — |
| 58 | Human | 19 | — | — |
| 59 | Human | 19 | — | — |
| 60 | Human | 19 | — | — |
| 61 | Human | 19 | — | — |
| 62 | Human | 19 | [1856-1874](18/19) | — |
| 63 | Human | 19 | — | — |
| 64 | Human | 19 | [1307-1318](12/12) | [1626-1637](12/12) |
| 65 | Human | 19 | — | — |
| 66 | Human | 19 | — | — |
| 67 | Human | 19 | [971-982](12/12) | [1290-1301](12/12) |
| 68 | Human | 19 | [931-947](17/17) | [1250-1266](17/17) |
| 69 | Human | 19 | [886-899](14/14) | [1205-1215](11/11) |
| 70 | Human | 19 | — | — |
| 71 | Human | 19 | [867-878](12/12) | [1186-1197](12/12) |
| 72 | Human | 19 | — | — |
| 73 | Human | 19 | — | — |
| 74 | Human | 19 | [838-856](18/19) | [1157-1169](13/13) |
| 75 | Human | 19 | [829-847](18/19) | [1148-1166](18/19) |
| 76 | Human | 19 | — | [1093-1106](14/14) |
| 77 | Human | 19 | — | — |
| 78 | Human | 19 | — | — |
| 79 | Human | 19 | — | — |
| 80 | Human | 19 | [661-679](18/19) | — |
| 81 | Human | 19 | [3208-3226](19/19) | — |
| 82 | Human | 19 | — | — |
| 83 | Human | 19 | — | — |
| 84 | Human | 19 | — | — |
| 85 | Human | 19 | — | — |
| 86 | Human | 19 | — | — |
| 87 | Human | 19 | — | — |
| 88 | Human | 19 | — | — |
| 89 | Human | 19 | — | — |
| 90 | Human | 19 | — | — |
| 91 | Human | 19 | — | — |
| 92 | Human | 19 | — | — |
| 93 | Human | 19 | — | — |
| 94 | Human | 19 | — | — |
| 95 | Human | 19 | — | — |
| 96 | Human | 19 | — | — |
| 97 | Human | 19 | — | — |
| 98 | Human | 19 | — | — |
| 99 | Human | 19 | — | — |
| 100 | Human | 19 | [2532-2545](14/14) | — |
| 101 | Human | 19 | — | — |
| 102 | Human | 19 | — | — |
| 103 | Human | 19 | — | — |
| 104 | Human | 19 | — | — |
| 105 | Human | 19 | — | — |
| 106 | Human | 19 | — | — |
| 107 | Human | 19 | — | — |
| 108 | Human | 19 | — | — |
| 109 | Human | 19 | — | — |
| 110 | Human | 19 | — | — |
| 111 | Human | 19 | — | — |
| 112 | Human | 19 | — | — |
| 113 | Human | 19 | — | — |
| 114 | Human | 19 | — | — |
| 115 | Human | 19 | — | — |
| 116 | Human | 19 | — | — |
| 117 | Human | 19 | — | — |
| 118 | Human | 19 | — | — |
| 119 | Human | 19 | [1613-1624](12/12) | — |
| 120 | Human | 19 | — | — |
| 121 | Human | 19 | — | — |
| 122 | Human | 19 | — | — |
| 123 | Human | 19 | — | — |
| 124 | Human | 19 | — | — |
| 125 | Human | 19 | — | — |
| 126 | Human | 19 | [1353-1369](16/17) | [1672-1688](17/17) |
| 127 | Human | 19 | — | — |
| 128 | Human | 19 | [1332-1345](14/14) | [1651-1664](14/14) |
| 129 | Human | 19 | — | — |
| 130 | Human | 19 | [1305-1318](14/14) | [1624-1637](14/14) |
| 131 | Human | 19 | [1300-1318](18/19) | [1619-1637](18/19) |
| 132 | Human | 19 | — | — |
| 133 | Human | 19 | — | — |
| 134 | Human | 19 | — | — |
| 135 | Human | 19 | — | — |
| 136 | Human | 19 | — | [1574-1584](11/11) |
| 137 | Human | 19 | [2427-2437](11/11) | — |
| 138 | Human | 19 | — | [1514-1524](11/11) |
| 139 | Human | 19 | [1159-1177](18/19) | [1483-1496](14/14) |
| 140 | Human | 19 | — | — |
| 141 | Human | 19 | — | — |
| 142 | Human | 19 | — | — |
| 143 | Human | 19 | — | — |
| 144 | Human | 19 | — | — |
| 145 | Human | 19 | — | — |
| 146 | Human | 19 | — | — |
| 147 | Human | 19 | — | — |
| 148 | Human | 19 | — | — |
| 149 | Human | 19 | [1071-1081](11/11) | — |

FOR 21
STRETCHES:

| | | | | |
|---|---|---|---|---|
| 1 | Cross Sp | 21 | [924-944](21/21) | [1243-1263](21/21) |
| 2 | Cross Sp | 21 | [927-947](21/21) | [1246-1266](21/21) |
| 3 | Cross Sp | 21 | [921-941](21/21) | [1240-1260](21/21) |
| 4 | Cross Sp | 21 | [920-940](21/21) | [1239-1259](21/21) |
| 5 | Cross Sp | 21 | [919-939](21/21) | [1238-1258](21/21) |
| 6 | Cross Sp | 21 | [527-547](21/21) | [846-866](21/21) |
| 7 | Cross Sp | 21 | [582-602](21/21) | [901-921](21/21) |
| 8 | Cross Sp | 21 | [925-945](21/21) | [1244-1264](21/21) |
| 9 | Cross Sp | 21 | [923-943](21/21) | [1242-1262](21/21) |
| 10 | Cross Sp | 21 | [580-600](21/21) | [899-919](21/21) |
| 11 | Cross Sp | 21 | [918-938](21/21) | [1237-1257](21/21) |
| 12 | Cross Sp | 21 | [581-601](21/21) | [900-920](21/21) |
| 13 | Cross Sp | 21 | [926-946](21/21) | [1245-1265](21/21) |
| 14 | Cross Sp | 21 | [525-545](21/21) | [844-864](21/21) |
| 15 | Cross Sp | 21 | [922-942](21/21) | [1241-1261](21/21) |
| 16 | Cross Sp | 21 | [526-546](21/21) | [845-865](21/21) |
| 17 | Cross Sp | 21 | [585-605](21/21) | [904-924](21/21) |
| 18 | Cross Sp | 21 | [595-615](21/21) | [914-934](21/21) |
| 19 | Cross Sp | 21 | [584-604](21/21) | [903-923](21/21) |
| 20 | Cross Sp | 21 | [583-603](21/21) | [902-922](21/21) |
| 21 | Cross Sp | 21 | [592-612](21/21) | [911-931](21/21) |
| 22 | Cross Sp | 21 | [1165-1185](21/21) | [1484-1504](21/21) |
| 23 | Cross Sp | 21 | [609-629](21/21) | [928-948](21/21) |
| 24 | Cross Sp | 21 | [608-628](21/21) | [927-947](21/21) |
| 25 | Cross Sp | 21 | [1166-1186](21/21) | [1485-1505](21/21) |
| 26 | Cross Sp | 21 | [610-630](21/21) | [929-949](21/21) |
| 27 | Cross Sp | 21 | [1164-1184](21/21) | [1483-1503](21/21) |
| 28 | Cross Sp | 21 | [1011-1031](21/21) | [1330-1350](21/21) |
| 29 | Cross Sp | 21 | [607-627](21/21) | [926-946](21/21) |
| 30 | Cross Sp | 21 | [611-631](21/21) | [930-950](21/21) |
| 31 | Cross Sp | 21 | [1012-1032](21/21) | [1331-1351](21/21) |
| 32 | Human | 21 | [1057-1077](20/21) | [1378-1394](16/17) |
| 33 | Human | 21 | — | — |
| 34 | Human | 21 | — | — |
| 35 | Human | 21 | — | — |
| 36 | Human | 21 | — | — |
| 37 | Human | 21 | [1299-1318](19/20) | [1618-1637](19/20) |
| 38 | Human | 21 | — | [1565-1584](19/20) |
| 39 | Human | 21 | — | — |
| 40 | Human | 21 | — | — |
| 41 | Human | 21 | — | — |
| 42 | Human | 21 | [970-982](13/13) | [1289-1301](13/13) |
| 43 | Human | 21 | [964-980](17/17) | [1283-1299](17/17) |
| 44 | Human | 21 | — | — |
| 45 | Human | 21 | — | — |
| 46 | Human | 21 | [935-955](20/21) | [1254-1274](20/21) |
| 47 | Human | 21 | [912-932](20/21) | [1231-1251](20/21) |
| 48 | Human | 21 | — | — |
| 49 | Human | 21 | [762-781](19/20) | [1081-1101](20/21) |
| 50 | Human | 21 | [510-521](12/12) | [829-840](12/12) |
| 51 | Human | 21 | [3207-3227](21/21) | — |
| 52 | Human | 21 | [3205-3223](19/19) | — |
| 53 | Human | 21 | [3205-3220](16/16) | — |
| 54 | Human | 21 | — | — |
| 55 | Human | 21 | — | — |
| 56 | Human | 21 | — | — |
| 57 | Human | 21 | — | — |
| 58 | Human | 21 | — | — |
| 59 | Human | 21 | — | — |
| 60 | Human | 21 | — | — |
| 61 | Human | 21 | — | — |
| 62 | Human | 21 | — | — |
| 63 | Human | 21 | — | — |
| 64 | Human | 21 | — | — |
| 65 | Human | 21 | — | — |
| 66 | Human | 21 | — | — |
| 67 | Human | 21 | — | — |
| 68 | Human | 21 | — | — |
| 69 | Human | 21 | − | — |
| 70 | Human | 21 | — | — |
| 71 | Human | 21 | — | — |
| 72 | Human | 21 | — | — |
| 73 | Human | 21 | — | — |
| 74 | Human | 21 | — | — |
| 75 | Human | 21 | — | — |
| 76 | Human | 21 | — | — |
| 77 | Human | 21 | — | — |
| 78 | Human | 21 | — | — |
| 79 | Human | 21 | — | — |
| 80 | Human | 21 | — | — |
| 81 | Human | 21 | — | — |
| 82 | Human | 21 | — | — |
| 83 | Human | 21 | — | — |
| 84 | Human | 21 | — | — |
| 85 | Human | 21 | — | — |
| 86 | Human | 21 | [1856-1874](18/19) | — |
| 87 | Human | 21 | [1855-1874](19/20) | — |
| 88 | Human | 21 | [1854-1868](15/15) | — |
| 89 | Human | 21 | — | — |
| 90 | Human | 21 | — | — |
| 91 | Human | 21 | — | — |
| 92 | Human | 21 | — | — |
| 93 | Human | 21 | — | — |
| 94 | Human | 21 | — | — |
| 95 | Human | 21 | [1359-1375](16/17) | [1675-1694](19/20) |
| 96 | Human | 21 | [1352-1369](17/18) | [1671-1688](18/18) |
| 97 | Human | 21 | — | — |
| 98 | Human | 21 | [1305-1318](14/14) | [1624-1637](14/14) |
| 99 | Human | 21 | [1297-1316](19/20) | [1616-1635](19/20) |
| 100 | Human | 21 | — | — |
| 101 | Human | 21 | — | — |
| 102 | Human | 21 | [1239-1253](15/15) | [1562-1572](11/11) |
| 103 | Human | 21 | — | [1514-1524](11/11) |
| 104 | Human | 21 | — | [1514-1524](11/11) |
| 105 | Human | 21 | — | — |
| 106 | Human | 21 | — | — |
| 107 | Human | 21 | — | — |
| 108 | Human | 21 | — | — |
| 109 | Human | 21 | — | — |
| 110 | Human | 21 | [1071-1081](11/11) | — |
| 111 | Human | 21 | [1063-1081](18/19) | — |
| 112 | Human | 21 | [1056-1069](14/14) | [1378-1388](11/11) |
| 113 | Human | 21 | — | — |
| 114 | Human | 21 | — | — |
| 115 | Human | 21 | — | [1318-1334](16/17) |
| 116 | Human | 21 | — | [1318-1333](15/16) |
| 117 | Human | 21 | — | [1318-1328](11/11) |
| 118 | Human | 21 | — | — |
| 119 | Human | 21 | — | — |
| 120 | Human | 21 | [2325-2335](11/11) | — |
| 121 | Human | 21 | [965-982](18/18) | [1284-1301](118/18) |
| 122 | Human | 21 | — | — |
| 123 | Human | 21 | — | — |
| 124 | Human | 21 | — | — |
| 125 | Human | 21 | [937-957](20/21) | [1256-1276](20/21) |
| 126 | Human | 21 | [932-952](20/21) | [1251-1271](20/21) |
| 127 | Human | 21 | [930-947](18/18) | [1249-1266](18/18) |
| 128 | Human | 21 | [929-947](19/19) | [1248-1266](19/19) |

Table J below shows further siRNA compounds specific for the SPTLC2 gene that have been selected according to the present invention.

TABLE J

| | | | | | | |
|---|---|---|---|---|---|---|
| | | Oligo | | | | |
| No | Source | Length | Sense siRNA | AntiSense siRNA | human 31881646 | mouse 31543768 |

FOR 19
STRETCHES:

| | | | | | | |
|---|---|---|---|---|---|---|
| 1 | Cross Sp | 19 | GCAAGAAGGAGCUGAUAGA | UCUAUCAGCUCCUUCUUGC | [1357-1375](19/19) | [1223-1241](19/19) |
| 2 | Cross Sp | 19 | GAACAUUCCUGCUCUUGUU | AACAAGAGCAGGAAUGUUC | [920-338](19/19) | [786-804](19/19) |
| 3 | Cross Sp | 19 | CUACAACUAUCUUGGAUUU | AAAUCCAAGAUAGUUGUAG | [713-731](19/19) | [579-597](19/19) |
| 4 | Cross Sp | 19 | GCAGCUCAUACCAAAGAAA | UUUCUUUGGUAUGAGCUGC | [1728-1746](19/19) | [1594-1612](19/19) |
| 5 | Cross Sp | 19 | AGAAGGAGCUGAUAGACUA | UAGUCUAUCAGCUCCUUCU | [1360-1378](19/19) | [1226-1244](19/19) |
| 6 | Cross Sp | 19 | ACAAGGCAUACUUGUAUCU | AGAUACAAGUAUGCCUUGU | [1198-1216](19/19) | [1064-1082](19/19) |
| 7 | Cross Sp | 19 | AGAAAUACAAGGCAUACUU | AAGUAUGCCUUGUAUUUCU | [1192-1210](19/19) | [1058-1076](19/19) |
| 8 | Cross Sp | 19 | AGAUGCCAUUGUUUAUGGU | ACCAUAAACAAUGGCAUCA | [1067-1085](19/19) | [933-951](19/19) |
| 9 | Cross Sp | 19 | UCAAACACAACAAUAUGCA | UGCAUAUUGUUGUGUUUGA | [1027-1045](19/19) | [893-911](19/19) |
| 10 | Cross Sp | 19 | GUCAGCAGCUCAUACCAAA | UUUGGUAUGAGCUGCUGAC | [1724-1742](19/19) | [1590-1608](19/19) |
| 11 | Cross Sp | 19 | CUGUCAGCAGCUCAUACCA | UGGUAUGAGCUGCUGACAG | [1722-1740](19/19) | [1588-1606](19/19) |
| 12 | Cross Sp | 19 | GAGGCAAGAAGGAGCUGAU | AUCAGCUCCUUCUUGCCUC | [1354-1372](19/19) | [1220-1238](19/19) |
| 13 | Cross Sp | 19 | AUACAAGGCAUACUUGUAU | AUACAAGUAUGCCUUGUAU | [1196-1214](19/19) | [1062-1080](19/19) |
| 14 | Cross Sp | 19 | UGGGUUCCUACAACUAUCU | AGAUAGUUGUAGGAACCCA | [706-724](19/19) | [572-590](19/19) |
| 15 | Cross Sp | 19 | AGAAGAAAUACAAGGCAUA | UAUGCCUUGUAUUUCUUCU | [1189-1207](19/19) | [1055-1073](19/19) |
| 16 | Cross Sp | 19 | ACAUGGGUUCCUACAACUA | UAGUUGUAGGAACCCAUGU | [703-721](19/19) | [569-587](19/19) |
| 17 | Cross Sp | 19 | GAAUCUUCAAACACAACAA | UUGUUGUGUUUGAAGAUUC | [1021-1039](19/19) | [887-905](19/19) |
| 18 | Cross Sp | 19 | GCCUGAUUCUGAGUGAUGA | UCAUCACUCAGAAUCAGGC | [949-967](19/19) | [815-833](19/19) |
| 19 | Cross Sp | 19 | AACAUGGGUUCCUACAACU | AGUUGUAGGAACCCAUGUU | [702-720](19/19) | [568-586](19/19) |
| 20 | Cross Sp | 19 | CCUACAACUAUCUUGGAUU | AAUCCAAGAUAGUUGUAGG | [712-730](19/19) | [578-596](19/19) |
| 21 | Cross Sp | 19 | CAGCUCAUACCAAAGAAAU | AUUUCUUUGGUAUGAGCUG | [1729-1747](19/19) | [1595-1613](19/19) |
| 22 | Cross Sp | 19 | CAGCAGCUCAUACCAAAGA | UCUUUGGUAUGAGCUGCUG | [1726-1744](19/19) | [1592-1610](19/19) |
| 23 | Cross Sp | 19 | GUUCCUACAACUAUCUUGG | CCAAGAUAGUUGUAGGAAC | [709-727](19/19) | [575-593](19/19) |
| 24 | Cross Sp | 19 | UUGCCUGUCAGCAGCUCAU | AUGAGCUGCUGACAGGCAA | [1718-1736](19/19) | [1584-1602](19/19) |
| 25 | Cross Sp | 19 | CCAGGUAUUUCAGGAGACG | CGUCUCCUGAAAUACCUGG | [1525-1543](19/19) | [1391-1409](19/19) |
| 26 | Cross Sp | 19 | CACCAGGUAUUUCAGGAGA | UCUCCUGAAAUACCUGGUG | [1523-1541](19/19) | [1389-1407](19/19) |
| 27 | Cross Sp | 19 | GGUUCCUACAACUAUCUUG | CAAGAUAGUUGUAGGAACC | [708-726](19/19) | [574-592](19/19) |
| 28 | Cross Sp | 19 | CAAGAAGGAGCUGAUAGAC | GUCUAUCAGCUCCUUCUUG | [1358-1376](19/19) | [1224-1242](19/19) |
| 29 | Cross Sp | 19 | GGGUUCCUACAACUAUCUU | AAGAUAGUUGUAGGAACCC | [707-725](19/19) | [573-591](19/19) |
| 30 | Cross Sp | 19 | AAAUACAAGGCAUACUUGU | ACAAGUAUGCCUUGUAUUU | [1194-1212](19/19) | [1060-1078](19/19) |
| 31 | Cross Sp | 19 | GAAAUACAAGGCAUACUUG | CAAGUAUGCCUUGUAUUUC | [1193-1211](19/19) | [1059-1077](19/19) |
| 32 | Cross Sp | 19 | CAUUGUUUAUGGUCAGCCU | AGGCUGACCAUAAACAAUG | [1073-1091](19/19) | [939-957](19/19) |
| 33 | Cross Sp | 19 | CAUGGGUUCCUACAACUAU | AUAGUUGUAGGAACCCAUG | [704-722](19/19) | [570-588](19/19) |
| 34 | Cross Sp | 19 | CAAACACAACAAUAUGCAA | UUGCAUAUUGUUGUGUUUG | [1028-1046](19/19) | [894-912](19/19) |
| 35 | Cross Sp | 19 | UUGCCUGAUUCUGAGUGAU | AUCACUCAGAAUCAGGCAA | [947-965](19/19) | [813-831](19/19) |
| 36 | Cross Sp | 19 | GUUGCCUGAUUCUGAGUGA | UCACUCAGAAUCAGGCAAC | [946-964](19/19) | [812-830](19/19) |
| 37 | Cross Sp | 19 | UCCUGCUCUUGUUGGCAAA | UUUGCCAACAAGAGCAGGA | [926-944](19/19) | [792-810](19/19) |
| 38 | Cross Sp | 19 | UGAACAUUCCUGCUCUUGU | ACAAGAGCAGGAAUGUUCA | [919-937](19/19) | [785-803](19/19) |
| 39 | Cross Sp | 19 | AAUGAACAUUCCUGCUCUU | AAGAGCAGGAAUGUUCAUU | [917-935](19/19) | [783-801](19/19) |
| 40 | Cross Sp | 19 | CAAUGAACAUUCCUGCUCU | AGAGCAGGAAUGUUCAUUG | [916-934](19/19) | [782-800](19/19) |
| 41 | Cross Sp | 19 | AAACAUGGGUUCCUACAAC | GUUGUAGGAACCCAUGUUU | [701-719](19/19) | [567-585](19/19) |
| 42 | Cross Sp | 19 | UAUGGCAUGGGAUUUGCAA | UUGCAAAUCCCAUGCCAUA | [891-909](19/19) | [757-775](19/19) |
| 43 | Cross Sp | 19 | UCCUACAACUAUCUUGGAU | AUCCAAGAUAGUUGUAGGA | [711-729](19/19) | [577-595](19/19) |
| 44 | Cross Sp | 19 | AGCAGCUCAUACCAAAGAA | UUCUUUGGUAUGAGCUGCU | [1727-1745](19/19) | [1593-1611](19/19) |
| 45 | Cross Sp | 19 | UGCCUGUCAGCAGCUCAUA | UAUGAGCUGCUGACAGGCA | [1719-1737](19/19) | [1585-1603](19/19) |
| 46 | Cross Sp | 19 | ACACCAGGUAUUUCAGGAG | CUCCUGAAAUACCUGGUGU | [1522-1540](19/19) | [1388-1406](19/19) |
| 47 | Cross Sp | 19 | AACACCAGGUAUUUCAGGA | UCCUGAAAUACCUGGUGUU | [1521-1539](19/19) | [1387-1405](19/19) |
| 48 | Cross Sp | 19 | AAGAAGGAGCUGAUAGACU | AGUCUAUCAGCUCCUUCUU | [1359-1377](19/19) | [1225-1243](19/19) |
| 49 | Cross Sp | 19 | GGCAAGAAGGAGCUGAUAG | CUAUCAGCUCCUUCUUGCC | [1356-1374](19/19) | [1222-1240](19/19) |
| 50 | Cross Sp | 19 | AGGCAAGAAGGAGCUGAUA | UAUCAGCUCCUUCUUGCCU | [1355-1373](19/19) | [1221-1239](19/19) |
| 51 | Cross Sp | 19 | UACAAGGCAUACUUGUAUC | GAUACAAGUAUGCCUUGUA | [1197-1215](19/19) | [1063-1081](19/19) |
| 52 | Cross Sp | 19 | AAGAAAUACAAGGCAUACU | AGUAUGCCUUGUAUUUCUU | [1191-1209](19/19) | [1057-1075](19/19) |
| 53 | Cross Sp | 19 | GAAGAAAUACAAGGCAUAC | GUAUGCCUUGUAUUUCUUC | [1190-1208](19/19) | [1056-1074](19/19) |
| 54 | Cross Sp | 19 | AAGAAGAAAUACAAGGCAU | AUGCCUUGUAUUUCUUCUU | [1188-1206](19/19) | [1054-1072](19/19) |
| 55 | Cross Sp | 19 | UUAUGGUCAGCCUCGGACA | UGUCCGAGGCUGACCAUAA | [1079-1097](19/19) | [945-963](19/19) |
| 56 | Cross Sp | 19 | AUGGGUUCCUACAACUAUC | GAUAGUUGUAGGAACCCAU | [705-723](19/19) | [571-589](19/19) |
| 57 | Cross Sp | 19 | CCAUUGUUUAUGGUCAGCC | GGCUGACCAUAAACAAUGG | [1072-1090](19/19) | [938-956](19/19) |
| 58 | Cross Sp | 19 | AUGCCAUUGUUUAUGGUCA | UGACCAUAAACAAUGGCAU | [1069-1087](19/19) | [935-953](19/19) |
| 59 | Cross Sp | 19 | GAUGCCAUUGUUUAUGGUC | GACCAUAAACAAUGGCAUC | [1068-1086](19/19) | [934-952](19/19) |
| 60 | Cross Sp | 19 | AAGAUGCCAUUGUUUAUGG | CCAUAAACAAUGGCAUCUU | [1066-1084](19/19) | [932-950](19/19) |
| 61 | Cross Sp | 19 | AAAGAUGCCAUUGUUUAUG | CAUAAACAAUGGCAUCUUU | [1065-1083](19/19) | [931-949](19/19) |
| 62 | Cross Sp | 19 | ACACAACAAUAUGCAAAGC | GCUUUGCAUAUUGUUGUGU | [1031-1049](19/19) | [897-915](19/19) |
| 63 | Cross Sp | 19 | AACACAACAAUAUGCAAAG | CUUUGCAUAUUGUUGUGUU | [1030-1048](19/19) | [896-914](19/19) |
| 64 | Cross Sp | 19 | UUCAAACACAACAAUAUGC | GCAUAUUGUUGUGUUUGAA | [1026-1044](19/19) | [892-910](19/19) |
| 65 | Cross Sp | 19 | CUUCAAACACAACAAUAUG | CAUAUUGUUGUGUUUGAAG | [1025-1043](19/19) | [891-909](19/19) |
| 66 | Cross Sp | 19 | UUCCUGCUCUUGUUGGCAA | UUGCCAACAAGAGCAGGAA | [925-943](19/19) | [791-809](19/19) |
| 67 | Cross Sp | 19 | CAUUCCUGCUCUUGUUGGC | GCCAACAAGAGCAGGAAUG | [923-941](19/19) | [789-807](19/19) |
| 68 | Cross Sp | 19 | ACAUUCCUGCUCUUGUUGG | CCAACAAGAGCAGGAAUGU | [922-940](19/19) | [788-806](19/19) |
| 69 | Cross Sp | 19 | AACAUUCCUGCUCUUGUUG | CAACAAGAGCAGGAAUGUU | [921-939](19/19) | [787-805](19/19) |
| 70 | Cross Sp | 19 | UCAAUGAACAUUCCUGCUC | GAGCAGGAAUGUUCAUUGA | [915-933](19/19) | [781-799](19/19) |
| 71 | Cross Sp | 19 | AUUCAAUGAACAUUCCUGC | GCAGGAAUGUUCAUUGAAU | [913-931](19/19) | [779-797](19/19) |
| 72 | Cross Sp | 19 | ACAACUAUCUUGGAUUUGC | GCAAAUCCAAGAUAGUUGU | [715-733](19/19) | [581-599](19/19) |
| 73 | Cross Sp | 19 | UCAGCAGCUCAUACCAAAG | CUUUGGUAUGAGCUGCUGA | [1725-1743](19/19) | [1591-1609](19/19) |
| 74 | Cross Sp | 19 | UGUCAGCAGCUCAUACCAA | UUGGUAUGAGCUGCUGACA | [1723-1741](19/19) | [1589-1607](19/19) |
| 75 | Cross Sp | 19 | UAAACAUGGGUUCCUACAA | UUGUAGGAACCCAUGUUUA | [700-718](19/19) | [566-584](19/19) |
| 76 | Cross Sp | 19 | AUAAACAUGGGUUCCUACA | UGUAGGAACCCAUGUUUAU | [699-717](19/19) | [565-583](19/19) |
| 77 | Cross Sp | 19 | UUUGCCUGUCAGCAGCUCA | UGAGCUGCUGACAGGCAAA | [1717-1735](19/19) | [1583-1601](19/19) |
| 78 | Cross Sp | 19 | ACCAGGUAUUUCAGGAGAC | GUCUCCUGAAAUACCUGGU | [1524-1542](19/19) | [1390-1408](19/19) |
| 79 | Cross Sp | 19 | AAUACAAGGCAUACUUGUA | UACAAGUAUGCCUUGUAUU | [1195-1213](19/19) | [1061-1079](19/19) |
| 80 | Cross Sp | 19 | UUUAUGGUCAGCCUCGGAC | GUCCGAGGCUGACCAUAAA | [1078-1096](19/19) | [944-962](19/19) |
| 81 | Cross Sp | 19 | GUUUAUGGUCAGCCUCGGA | UCCGAGGCUGACCAUAAAC | [1077-1095](19/19) | [943-961](19/19) |
| 82 | Cross Sp | 19 | UGUUUAUGGUCAGCCUCGG | CCGAGGCUGACCAUAAACA | [1076-7094](19/19) | [942-960](19/19) |
| 83 | Cross Sp | 19 | AUUGUUUAUGGUCAGCCUC | GAGGCUGACCAUAAACAAU | [1074-1092](19/19) | [940-958](19/19) |
| 84 | Cross Sp | 19 | GCCAUUGUUUAUGGUCAGC | GCUGACCAUAAACAAUGGC | [1071-1089](19/19) | [937-955](19/19) |
| 85 | Cross Sp | 19 | UGCCAUUGUUUAUGGUCAG | CUGACCAUAAACAAUGGCA | [1070-1088](19/19) | [936-954](19/19) |
| 86 | Cross Sp | 19 | UGCCUGAUUCUGAGUGAUG | CAUCACUCAGAAUCAGGCA | [948-966](19/19) | [814-832](19/19) |
| 87 | Cross Sp | 19 | CCUGCUCUUGUUGGCAAAG | CUUUGCCAACAAGAGCAGG | [927-945](19/19) | [793-811](19/19) |
| 88 | Cross Sp | 19 | AUGAACAUUCCUGCUCUUG | CAAGAGCAGGAAUGUUCAU | [918-936](19/19) | [784-802](19/19) |
| 89 | Cross Sp | 19 | UUCAAUGAACAUUCCUGCU | AGCAGGAAUGUUCAUUGAA | [914-932](19/19) | [780-798](19/19) |
| 90 | Cross Sp | 19 | AAUUCAAUGAACAUUCCUG | CAGGAAUGUUCAUUGAAUU | [912-930](19/19) | [778-796](19/19) |
| 91 | Cross Sp | 19 | AUGGCAUGGGAUUUGCAAC | GUUGCAAAUCCCAUGCCAU | [892-910](19/19) | [758-776](19/19) |
| 92 | Cross Sp | 19 | UUCCUACAACUAUCUUGGA | UCCAAGAUAGUUGUAGGAA | [710-728](19/19) | [576-594](19/19) |
| 93 | Cross Sp | 19 | UUUUGCCUGUCAGCAGCUC | GAGCUGCUGACAGGCAAAA | [1716-1734](19/19) | [1582-1600](19/19) |
| 94 | Cross Sp | 19 | UUGUUUAUGGUCAGCCUCG | CGAGGCUGACCAUAAACAA | [1075-1093](19/19) | [941-959](19/19) |
| 95 | Cross Sp | 19 | AUUCCUGCUCUUGUUGGCA | UGCCAACAAGAGCAGGAAU | [924-942](19/19) | [790-808](19/19) |
| 96 | Cross Sp | 19 | UACAACUAUCUUGGAUUUG | CAAAUCCAAGAUAGUUGUA | [714-732](19/19) | [580-598](19/19) |
| 97 | Cross Sp | 19 | CCUGUCAGCAGCUCAUACC | GGUAUGAGCUGCUGACAGG | [1721-1739](19/19) | [1587-1605](19/19) |
| 98 | Cross Sp | 19 | GGAGGCAAGAAGGAGCUGA | UCAGCUCCUUCUUGCCUCC | [1353-1371](19/19) | [1219-1237](19/19) |
| 99 | Cross Sp | 19 | GCCUGUCAGCAGCUCAUAC | GUAUGAGCUGCUGACAGGC | [1720-1738](19/19) | [1586-1604](19/19) |
| 100 | Cross Sp | 19 | AUCAUGGGGCAGGAUGGCA | UGCCAUCCUGCCCCAUGAU | [1464-1482](19/19) | [1330-1348](19/19) |
| 101 | Human | 19 | GAAGAAAAUUCUCAUCCUU | AAGGAUGAGAAUUUUCUUC | [1109-1127](19/19) | — |
| 102 | Human | 19 | CGAAGGCCCUGGAAGAAAA | UUUUCUUCCAGGGCCUUCG | [1098-1116](19/19) | [965-982](17/18) |
| 103 | Human | 19 | CCAUUAGAAUCUUCAAACA | UGUUUGAAGAUUCUAAUGG | [1015-1033](19/19) | [881-899](18/19) |
| 104 | Human | 19 | GCAACCAUUAGAAUCUUCA | UGAAGAUUCUAAUGGUUGC | [1011-1029](19/19) | — |
| 105 | Human | 19 | GCAACGAAUUCAAUGAACA | UGUUCAUUGAAUUCGUUGC | [906-924](19/19) | [772-790](18/19) |
| 106 | Human | 19 | GGAUUUGCAACGAAUUCAA | UUGAAUUCGUUGCAAAUCC | [900-918](19/19) | [766-784](18/19) |
| 107 | Human | 19 | GGACAAGCAUGAAGAACUA | UAGUUCUUCAUGCUUGUCC | [827-845](19/19) | [693-711](19/19) |
| 108 | Human | 19 | CCACAUUCAUGAAGAUUAU | AUAAUCUUCAUGAAUGUGG | [5901-5919](19/19) | — |
| 109 | Human | 19 | GCAUUGCUAUGACCUAAUU | AAUUAGGUCAUAGCAAUGC | [5766-5784](19/19) | [747-758](12/12) |
| 110 | Human | 19 | GGAAAAAAGUGAUGACAUU | AAUGUCAUCACUUUUUUCC | [5570-5588](19/19) | — |
| 111 | Human | 19 | CAACUAUCUUGGAUUUGCA | UGCAAAUCCAAGAUAGUUG | [716-734](19/19) | [582-599](18/18) |
| 112 | Human | 19 | CAACUCUAGAGAAAACCAA | UUGGUUUUCUCUAGAGUUG | [5255-5273](19/19) | — |
| 113 | Human | 19 | GUACCUUCAAGUUCUUGAU | AUCAAGAACUUGAAGGUAC | [5225-5243](19/19) | — |
| 114 | Human | 19 | GAACAUGCUUCCAGUGAUU | AAUCACUGGAAGCAUGUUC | [5160-5178](19/19) | — |
| 115 | Human | 19 | GAAGAAUCCAGCCAUCGUA | UACGAUGGCUGGAUUCUUC | [4990-5008](19/19) | — |
| 116 | Human | 19 | GGACUUUAUAGCACUUUUU | AAAAAGUGCUAUAAAGUCC | [4882-4900](19/19) | — |
| 117 | Human | 19 | GCAGGGGCAUGGACUUUAU | AUAAAGUCCAUGCCCCUGC | [4872-4890](19/19) | — |
| 118 | Human | 19 | CUAGUCCUUAGCACUUUUU | AAAAAGUGCUAAGGACUAG | [4671-4689](19/19) | — |
| 119 | Human | 19 | GCAUACAAGAGUAGUUUCU | AGAAACUACUCUUGUAUGC | [4545-4563](19/19) | — |
| 120 | Human | 19 | CCAAGUACUUGGAGGAAUA | UAUUCCUCCAAGUACUUGG | [4483-4501](19/19) | — |
| 121 | Human | 19 | CCAGUCAUAUCCUAGGUAU | AUACCUAGGAUAUGACUGG | [4454-4472](19/19) | — |
| 122 | Human | 19 | GAACUUACCUGCUAGUCUU | AAGACUAGCAGGUAAGUUC | [4188-4206](19/19) | [3515-3526](12/12) |
| 123 | Human | 19 | GCAAAACUGUGUCUCAAAA | UUUUGAGACACAGUUUUGC | [3656-3674](19/19) | — |
| 124 | Human | 19 | GGAACAGAAUGGAAAAACA | UGUUUUUCCAUUCUGUUCC | [3348-3366](19/19) | [2962-2972](11/11) |
| 125 | Human | 19 | CCAUGCAACAGAAAGAGAA | UUCUCUUUCUGUUGCAUGG | [488-506](19/19) | [354-372](19/19) |
| 126 | Human | 19 | GAAGGCCACUCUCACAUCU | AGAUGUGAGAGUGGCCUUC | [2672-2690](19/19) | — |
| 127 | Human | 19 | CCAUCAAAUACUUCUUCAU | AUGAAGAAGUAUUUGAUGG | [2532-2550](19/19) | — |
| 128 | Human | 19 | CCAAGGGUUUGGUCUUAUU | AAUAAGACCAAACCCUUGG | [2476-2494](19/19) | — |
| 129 | Human | 19 | GAAGUUUUCACCCUUUAAU | AUUAAAGGGUGAAAACUUC | [2392-2410](19/19) | — |
| 130 | Human | 19 | CAAGUGCCUUCCACUUACU | AGUAAGUGGAAGGCACUUG | [2304-2322](19/19) | [2164-2174](11/11) |
| 131 | Human | 19 | GAAGGUGACUCACUUUGCU | AGCAAAGUGAGUCACCUUC | [2091-2109](19/19) | — |
| 132 | Human | 19 | CCAGGAACCACACUUCUGU | ACAGAAGUGUGGUUCCUGG | [1947-1965](19/19) | [1812-1827](16/16) |
| 133 | Human | 19 | CUACAUGCCUGCCAAAAUU | AAUUUUGGCAGGCAUGUAG | [1607-1625](19/19) | — |
| 134 | Human | 19 | GGAGACGCCUGAAAGAGAU | AUCUCUUUCAGGCGUCUCC | [1537-1555](19/19) | [1403-1415](13/13) |
| 135 | Human | 19 | CCAGAUCCAUCAUGUUACA | UGUAACAUGAUGGAUCUGG | [317-335](19/19) | — |
| 136 | Human | 19 | GGAACGUUCACAAAGAGUU | AACUCUUUGUGAACGUUCC | [1311-1329](19/19) | [1177-1193](16/17) |
| 137 | Human | 19 | GGAUGAGGCUCACAGCAUU | AAUGCUGUGAGCCUCAUCC | [1217-1235](19/19) | [1083-1101](19/19) |
| 138 | Human | 19 | CCCUCAAGAAGAAAUACAA | UUGUAUUUCUUCUUGAGGG | [1183-1201](19/19) | [1051-1067](17/17) |
| 139 | Human | 19 | UGAUUGCCCUCAAGAAGAA | UUCUUCUUGAGGGCAAUCA | [1177-1195](19/19) | [1043-1061](18/19) |
| 140 | Human | 19 | GAAGUGAUUGCCCUCAAGA | UCUUGAGGGCAAUCACUUC | [1173-1191](19/19) | [1039-1057](18/19) |
| 141 | Human | 19 | CCUUGUGGAAGGAAUAUAU | AUAUAUUCCUUCCACAAGG | [1124-1142](19/19) | [990-1008](18/19) |
| 142 | Human | 19 | UCAUCCUUGUGGAAGGAAU | AUUCCUUCCACAAGGAUGA | [1120-1138](19/19) | [988-1001](14/14) |
| 143 | Human | 19 | GGCCCUGGAAGAAAAUUCU | AGAAUUUUCUUCCAGGGCC | [1102-1120](19/19) | [970-983](14/14) |
| 144 | Human | 19 | GAAGGCCCUGGAAGAAAAU | AUUUUCUUCCAGGGCCUUC | [1099-1117](19/19) | [965-983](18/19) |
| 145 | Human | 19 | GCCUAGAGAAGCUAUUGAA | UUCAAUAGCUUCUCUAGGC | [1048-1066](19/19) | — |
| 146 | Human | 19 | AAAGCCUAGAGAAGCUAUU | AAUAGCUUCUCUAGGCUUU | [1045-1063](19/19) | — |
| 147 | Human | 19 | GCAAAGCCUAGAGAAGCUA | UAGCUUCUCUAGGCUUUGC | [1043-1061](19/19) | — |
| 148 | Human | 19 | GGAGCAACCAUUAGAAUCU | AGAUUCUAAUGGUUGCUCC | [1008-1026](19/19) | [874-892](18/19) |
| 149 | Human | 19 | UCAGGAGCAACCAUUAGAA | UUCUAAUGGUUGCUCCUGA | [1005-1023](19/19) | [871-885](15/15) |
| 150 | Human | 19 | GCAAAGGUUGCCUGAUUCU | AGAAUCAGGCAACCUUUGC | [940-958](19/19) | [806-824](19/19) |
| 151 | Human | 19 | CGAAUUCAAUGAACAUUCC | GGAAUGUUCAUUGAAUUCG | [910-928](19/19) | [778-794](17/17) |
| 152 | Human | 19 | CAUGGGAUUUGCAACGAAU | AUUCGUUGCAAAUCCCAUG | [896-914](19/19) | [762-776](15/15) |
| 153 | Human | 19 | GCAUGGGAUUUGCAACGAA | UUCGUUGCAAAUCCCAUGC | [895-913](19/19) | [761-776](16/16) |
| 154 | Human | 19 | UGACCCUAAAGAAUGCAAA | UUUGCAUUCUUUAGGGUCA | [7217-7235](19/19) | — |
| 155 | Human | 19 | CAUGAUAAAUGGCCUUAAA | UUUAAGGCCAUUUAUCAUG | [7162-7180](19/19) | — |
| 156 | Human | 19 | ACAUAGUGCACAUGAUAAA | UUUAUCAUGUGCACUAUGU | [7152-7170](19/19) | — |
| 157 | Human | 19 | CUACAUAGUGCACAUGAUA | UAUCAUGUGCACUAUGUAG | [7150-7168](19/19) | — |
| 158 | Human | 19 | CUGGGGACAAGGUUUUGUU | AACAAAACCUUGUCCCCAG | [7129-7147](19/19) | — |
| 159 | Human | 19 | GAGCUGGGGACAAGGUUUU | AAAACCUUGUCCCCAGCUC | [7126-7144](19/19) | — |
| 160 | Human | 19 | GGAAGUGUCACAUUCACAG | CUGUGAAUGUGACACUUCC | [7083-7101](19/19) | — |
| 161 | Human | 19 | GACCCUAAUAGAAAUGUUU | AAACAUUUCUAUUAGGGUC | [7043-7061](19/19) | — |
| 162 | Human | 19 | GCUAGGUUAAGACCCUAAU | AUUAGGGUCUUAACCUAGC | [7033-7051](19/19) | — |
| 163 | Human | 19 | CCAGUGACAUCUGUUUUCU | AGAAAACAGAUGUCACUGG | [6981-6999](19/19) | — |
| 164 | Human | 19 | UGAUCCCUUUGCUUUUAAU | AUUAAAAGCAAAGGGAUCA | [6929-6947](19/19) | — |
| 165 | Human | 19 | CCAAUGAUCCCUUUGCUUU | AAAGCAAAGGGAUCAUUGG | [6925-6943](19/19) | — |
| 166 | Human | 19 | GCUAGUGCUAGCCAAUGAU | AUCAUUGGCUAGCACUAGC | [6914-6932](19/19) | — |
| 167 | Human | 19 | GCUUCCCUAUCACACAGAA | UUCUGUGUGAUAGGGAAGC | [6890-6908](19/19) | — |
| 168 | Human | 19 | CAGCCAGAGAGCCAAAUUA | UAAUUUGGCUCUCUGGCUG | [6863-6881](19/19) | — |
| 169 | Human | 19 | GACUCUGUCUCAAAAAAAA | UUUUUUUUGAGACAGAGUC | [6802-6820](19/19) | — |
| 170 | Human | 19 | GAGACUCUGUCUCAAAAAA | UUUUUUGAGACAGAGUCUC | [6800-6818](19/19) | — |
| 171 | Human | 19 | GGGAGACUCUGUCUCAAAA | UUUUGAGACAGAGUCUCCC | [6798-6816](19/19) | — |
| 172 | Human | 19 | GGAGCUUGUAGCAAGGUUC | GAACCUUGCUACAAGCUCC | [848-866](19/19) | [721-732](12/12) |
| 173 | Human | 19 | CUGAGGCACAGGAAUUGCU | AGCAAUUCCUGUGCCUCAG | [6712-6730](19/19) | — |
| 174 | Human | 19 | GCACAUGCCUGUAAUCCCA | UGGGAUUACAGGCAUGUGC | [6679-6697](19/19) | — |
| 175 | Human | 19 | GGUGGCACAUGCCUGUAAU | AUUACAGGCAUGUGCCACC | [6675-6693](19/19) | — |
| 176 | Human | 19 | GAAACCUCAUCUCUACUAA | UUAGUAGAGAUGAGGUUUC | [6633-6651](19/19) | — |
| 177 | Human | 19 | CGAAACCUCAUCUCUACUA | UAGUAGAGAUGAGGUUUCG | [6632-6650](19/19) | — |
| 178 | Human | 19 | CAAGCAUGAAGAACUAGAG | CUCUAGUUCUUCAUGCUUG | [830-848](19/19) | [696-714](19/19) |
| 179 | Human | 19 | AGAACAGUCUGGCCAACAU | AUGUUGGCCAGACUGUUCU | [6611-6629](19/19) | — |
| 180 | Human | 19 | GAAGCAGGCAGAUUGCUUG | CAAGCAAUCUGCCUGCUUC | [6578-6596](19/19) | — |
| 181 | Human | 19 | CACCUGUAAUCCCAACACU | AGUGUUGGGAUUACAGGUG | [6549-6567](19/19) | — |
| 182 | Human | 19 | CAACUUUAUCCACAUGAAA | UUUCAUGUGGAUAAAGUUG | [6502-6520](19/19) | — |
| 183 | Human | 19 | UCAACUUUAUCCACAUGAA | UUCAUGUGGAUAAAGUUGA | [6501-6519](19/19) | — |
| 184 | Human | 19 | CUCGCCUCAACUUUAUCCA | UGGAUAAAGUUGAGGCGAG | [6495-6513](19/19) | — |
| 185 | Human | 19 | CCUUCCAUGUGGAGAAAAA | UUUUUCUCCACAUGGAAGG | [6465-6483](19/19) | — |
| 186 | Human | 19 | GCAGCCAUCUUAAUACAUU | AAUGUAUUAAGAUGGCUGC | [6428-6446](19/19) | — |
| 187 | Human | 19 | CAGCAGCCAUCUUAAUACA | UGUAUUAAGAUGGCUGCUG | [6426-6444](19/19) | — |
| 188 | Human | 19 | GCAGGAAAUUGGAAACCUG | CAGGUUUCCAAUUUCCUGC | [809-827](19/19) | [676-693](18/18) |
| 189 | Human | 19 | CUACUUGCUUCACGUGGAU | AUCCACGUGAAGCAAGUAG | [6397-6415](19/19) | — |
| 190 | Human | 19 | GAACUUACAGGGAUUUUUU | AAAAAAUCCCUGUAAGUUC | [6348-6366](19/19) | — |
| 191 | Human | 19 | CAAGAACUUACAGGGAUUU | AAAUCCCUGUAAGUUCUUG | [6345-6363](19/19) | — |
| 192 | Human | 19 | GUACUCGGCAGGAAAUUGG | CCAAUUUCCUGCCGAGUAC | [802-820](19/19) | [670-686](16/17) |
| 193 | Human | 19 | GCAGUACUCGGCAGGAAAU | AUUUCCUGCCGAGUACUGC | [799-817](19/19) | — |
| 194 | Human | 19 | UGAAUGGGGAGGUAUAAAA | UUUUAUACCUCCCCAUUCA | [6298-6316](19/19) | — |

FOR 21
STRETCHES:

| | | | | | | |
|---|---|---|---|---|---|---|
| 1 | Cross Sp | 21 | GGAGGCAAGAAGGAGCUGAUA | UAUCAGCUCCUUCUUGCCUCC | [1353-1373](21/21) | [1219-1239](21/21) |
| 2 | Cross Sp | 21 | AAACAUGGGUUCCUACAACUA | UAGUUGUAGGAACCCAUGUUU | [701-721](21/21) | [567-587](21/21) |
| 3 | Cross Sp | 21 | GGUUCCUACAACUAUCUUGGA | UCCAAGAUAGUUGUAGGAACC | [708-728](21/21) | [574-594](21/21) |
| 4 | Cross Sp | 21 | AGCAGCUCAUACCAAAGAAAU | AUUUCUUUGGUAUGAGCUGCU | [1727-1747](21/21) | [1593-1613](21/21) |
| 5 | Cross Sp | 21 | CAGCAGCUCAUACCAAAGAAA | UUUCUUUGGUAUGAGCUGCUG | [1726-1746](21/21) | [1592-1612](21/21) |
| 6 | Cross Sp | 21 | UCAGCAGCUCAUACCAAAGAA | UUCUUUGGUAUGAGCUGCUGA | [1725-1745](21/21) | [1591-1611](21/21) |
| 7 | Cross Sp | 21 | CCUGUCAGCAGCUCAUACCAA | UUGGUAUGAGCUGCUGACAGG | [1721-1741](21/21) | [1587-1607](21/21) |
| 8 | Cross Sp | 21 | UUUGCCUGUCAGCAGCUCAUA | UAUGAGCUGCUGACAGGCAAA | [1717-1737](21/21) | [1583-1603](21/21) |
| 9 | Cross Sp | 21 | CAAGAAGGAGCUGAUAGACUA | UAGUCUAUCAGCUCCUUCUUG | [1358-1378](21/21) | [1224-1244](21/21) |
| 10 | Cross Sp | 21 | GCAAGAAGGAGCUGAUAGACU | AGUCUAUCAGCUCCUUCUUGC | [1357-1377](21/21) | [1223-1243](21/21) |
| 11 | Cross Sp | 21 | GGCAAGAAGGAGCUGAUAGAC | GUCUAUCAGCUCCUUCUUGCC | [1356-1376](21/21) | [1222-1242](21/21) |
| 12 | Cross Sp | 21 | AUACAAGGCAUACUUGUAUCU | AGAUACAAGUAUGCCUUGUAU | [1196-1216](21/21) | [1062-1082](21/21) |
| 13 | Cross Sp | 21 | AUGGGUUCCUACAACUAUCUU | AAGAUAGUUGUAGGAACCCAU | [705-725](21/21) | [571-591](21/21) |
| 14 | Cross Sp | 21 | GAAAUACAAGGCAUACUUGUA | UACAAGUAUGCCUUGUAUUUC | [1193-1213](21/21) | [1059-1079](21/21) |
| 15 | Cross Sp | 21 | AGAAAUACAAGGCAUACUUGU | ACAAGUAUGCCUUGUAUUUCU | [1192-1212](21/21) | [1058-1078](21/21) |
| 16 | Cross Sp | 21 | GAAGAAAUACAAGGCAUACUU | AAGUAUGCCUUGUAUUUCUUC | [1190-1210](21/21) | [1056-1076](21/21) |
| 17 | Cross Sp | 21 | CAUGGGUUCCUACAACUAUCU | AGAUAGUUGUAGGAACCCAUG | [704-724](21/21) | [570-590](21/21) |
| 18 | Cross Sp | 21 | CCAUUGUUUAUGGUCAGCCUC | GAGGCUGACCAUAAACAAUGG | [1072-1092](21/21) | [938-958](21/21) |
| 19 | Cross Sp | 21 | AAAGAUGCCAUUGUUUAUGGU | ACCAUAAACAAUGGCAUCUUU | [1065-1085](21/21) | [931-951](21/21) |
| 20 | Cross Sp | 21 | CUUCAAACACAACAAUAUGCA | UGCAUAUUGUUGUGUUUGAAG | [1025-1045](21/21) | [891-911](21/21) |
| 21 | Cross Sp | 21 | ACAUUCCUGCUCUUGUUGGCA | UGCCAACAAGAGCAGGAAUGU | [922-942](21/21) | [788-808](21/21) |
| 22 | Cross Sp | 21 | GAACAUUCCUGCUCUUGUUGG | CCAACAAGAGCAGGAAUGUUC | [920-940](21/21) | [786-806](21/21) |
| 23 | Cross Sp | 21 | UGAACAUUCCUGCUCUUGUUG | CAACAAGAGCAGGAAUGUUCA | [919-939](21/21) | [785-805](21/21) |
| 24 | Cross Sp | 21 | CAAUGAACAUUCCUGCUCUUG | CAAGAGCAGGAAUGUUCAUUG | [916-936](21/21) | [782-802](21/21) |
| 25 | Cross Sp | 21 | CCUACAACUAUCUUGGAUUUG | CAAAUCCAAGAUAGUUGUAGG | [712-732](21/21) | [578-598](21/21) |
| 26 | Cross Sp | 21 | GUCAGCAGCUCAUACCAAAGA | UCUUUGGUAUGAGCUGCUGAC | [1724-1744](21/21) | [1590-1610](21/21) |
| 27 | Cross Sp | 21 | UGUCAGCAGCUCAUACCAAAG | CUUUGGUAUGAGCUGCUGACA | [1723-1743](21/21) | [1589-1609](21/21) |
| 28 | Cross Sp | 21 | GGGUUCCUACAACUAUCUUGG | CCAAGAUAGUUGUAGGAACCC | [707-727](21/21) | [573-593](21/21) |
| 29 | Cross Sp | 21 | CUGUCAGCAGCUCAUACCAAA | UUUGGUAUGAGCUGCUGACAG | [1722-1742](21/21) | [1588-1608](21/21) |
| 30 | Cross Sp | 21 | AAGAAAUACAAGGCAUACUUG | CAAGUAUGCCUUGUAUUUCUU | [1191-1211](21/21) | [1057-1077](21/21) |
| 31 | Cross Sp | 21 | AAGAAGAAAUACAAGGCAUAC | GUAUGCCUUGUAUUUCUUCUU | [1188-1208](21/21) | [1054-1074](21/21) |
| 32 | Cross Sp | 21 | GUUUAUGGUCAGCCUCGGACA | UGUCCGAGGCUGACCAUAAAC | [1077-1097](21/21) | [943-963](21/21) |
| 33 | Cross Sp | 21 | AAGAUGCCAUUGUUUAUGGUC | GACCAUAAACAAUGGCAUCUU | [1066-1086](21/21) | [932-952](21/21) |
| 34 | Cross Sp | 21 | AAACACAACAAUAUGCAAAGC | GCUUUGCAUAUUGUUGUGUUU | [1029-1049](21/21) | [895-915](21/21) |
| 35 | Cross Sp | 21 | CAAACACAACAAUAUGCAAAG | CUUUGCAUAUUGUUGUGUUUG | [1028-1048](21/21) | [894-914](21/21) |
| 36 | Cross Sp | 21 | AACAUGGGUUCCUACAACUAU | AUAGUUGUAGGAACCCAUGUU | [702-722](21/21) | [568-588](21/21) |
| 37 | Cross Sp | 21 | GUUGCCUGAUUCUGAGUGAUG | CAUCACUCAGAAUCAGGCAAC | [946-966](21/21) | [812-832](21/21) |
| 38 | Cross Sp | 21 | UUCCUGCUCUUGUUGGCAAAG | CUUUGCCAACAAGAGCAGGAA | [925-945](21/21) | [791-811](21/21) |
| 39 | Cross Sp | 21 | UCAAUGAACAUUCCUGCUCUU | AAGAGCAGGAAUGUUCAUUGA | [915-935](21/21) | [781-801](21/21) |
| 40 | Cross Sp | 21 | UCCUACAACUAUCUUGGAUUU | AAAUCCAAGAUAGUUGUAGGA | [711-731](21/21) | [577-597](21/21) |
| 41 | Cross Sp | 21 | UUCCUACAACUAUCUUGGAUU | AAUCCAAUAUAGUUGUAGGAA | [710-730](21/21) | [576-596](21/21) |
| 42 | Cross Sp | 21 | GUUCCUACAACUAUCUUGGAU | AUCCAAGAUAGUUGUAGGAAC | [709-729](21/21) | [575-595](21/21) |
| 43 | Cross Sp | 21 | UAAACAUGGGUUCCUACAACU | AGUUGUAGGAACCCAUGUUUA | [700-720](21/21) | [566-586](21/21) |
| 44 | Cross Sp | 21 | CACCAGGUAUUUCAGGAGACG | CGUCUCCUGAAAUACCUGGUG | [1523-1543](21/21) | [1389-1409](21/21) |
| 45 | Cross Sp | 21 | ACACCAGGUAUUUCAGGAGAC | GUCUCCUGAAAUACCUGGUGU | [1522-1542](21/21) | [1388-1408](21/21) |
| 46 | Cross Sp | 21 | AACACCAGGUAUUUCAGGAGA | UCUCCUGAAAUACCUGGUGUU | [1521-1541](21/21) | [1387-1407](21/21) |
| 47 | Cross Sp | 21 | AGGCAAGAAGGAGCUGAUAGA | UCUAUCAGCUCCUUCUUGCCU | [1355-1375](21/21) | [1221-1241](21/21) |
| 48 | Cross Sp | 21 | GAGGCAAGAAGGAGCUGAUAG | CUAUCAGCUCCUUCUUGCCUC | [1354-1374](21/21) | [1220-1240](21/21) |
| 49 | Cross Sp | 21 | AGAAGAAAUACAAGGCAUACU | AGUAUGCCUUGUAUUUCUUCU | [1189-1209](21/21) | [1055-1075](21/21) |
| 50 | Cross Sp | 21 | UUGUUUAUGGUCAGCCUCGGA | UCCGAGGCUGACCAUAAACAA | [1075-1095](21/21) | [941-961](21/21) |
| 51 | Cross Sp | 21 | GCCAUUGUUUAUGGUCAGCCU | AGGCUGACCAUAAACAAUGGC | [1071-1091](21/21) | [937-957](21/21) |
| 52 | Cross Sp | 21 | GAUGCCAUUGUUUAUGGUCAG | CUGACCAUAAACAAUGGCAUC | [1068-1088](21/21) | [934-954](21/21) |
| 53 | Cross Sp | 21 | AGAUGCCAUUGUUUAUGGUCA | UGACCAUAAACAAUGGCAUCU | [1067-1087](21/21) | [933-953](21/21) |
| 54 | Cross Sp | 21 | ACAUGGGUUCCUACAACUAUC | GAUAGUUGUAGGAACCCAUGU | [703-723](21/21) | [569-589](21/21) |
| 55 | Cross Sp | 21 | UUGCCUGAUUCUGAGUGAUGA | UCAUCACUCAGAAUCAGGCAA | [947-967](21/21) | [813-833](21/21) |
| 56 | Cross Sp | 21 | AUUCCUGCUCUUGUUGGCAAA | UUUGCCAACAAGAGCAGGAAU | [924-944](21/21) | [790-810](21/21) |
| 57 | Cross Sp | 21 | AUGAACAUUCCUGCUCUUGUU | AACAAGAGCAGGAAUGUUCAU | [918-938](21/21) | [784-804](21/21) |
| 58 | Cross Sp | 21 | AAUGAACAUUCCUGCUCUUGU | ACAAGAGCAGGAAUGUUCAUU | [917-937](21/21) | [783-803](21/21) |
| 59 | Cross Sp | 21 | UUCAAUGAACAUUCCUGCUCU | AGAGCAGGAAUGUUCAUUGAA | [914-934](21/21) | [780-800](21/21) |
| 60 | Cross Sp | 21 | AAUUCAAUGAACAUUCCUGCU | AGCAGGAAUGUUCAUUGAAUU | [912-932](21/21) | [778-798](21/21) |
| 61 | Cross Sp | 21 | CUACAACUAUCUUGGAUUUGC | GCAAAUCCAAGAUAGUUGUAG | [713-733](21/21) | [579-599](21/21) |
| 62 | Cross Sp | 21 | UUGCCUGUCAGCAGCUCAUAC | GUAUGAGCUGCUGACAGGCAA | [1718-1738](21/21) | [1584-1604](21/21) |
| 63 | Cross Sp | 21 | UGGGUUCCUACAACUAUCUUG | CAAGAUAGUUGUAGGAACCCA | [706-726](21/21) | [572-592](21/21) |
| 64 | Cross Sp | 21 | AAUACAAGGCAUACUUGUAUC | GAUACAAGUAUGCCUUGUAUU | [1195-1215](21/21) | [1061-1081](21/21) |
| 65 | Cross Sp | 21 | CAUUGUUUAUGGUCAGCCUCG | CGAGGCUGACCAUAAACAAUG | [1073-1093](21/21) | [939-959](21/21) |
| 66 | Cross Sp | 21 | AUGCCAUUGUUUAUGGUCAGC | GCUGACCAUAAACAAUGGCAU | [1069-1089](21/21) | [935-955](21/21) |
| 67 | Cross Sp | 21 | UCUUCAAACACAACAAUAUGC | GCAUAUUGUUGUGUUUGAAGA | [1024-1044](21/21) | [890-910](21/21) |
| 68 | Cross Sp | 21 | AUAAACAUGGGUUCCUACAAC | GUUGUAGGAACCCAUGUUUAU | [699-719](21/21) | [565-585](21/21) |
| 69 | Cross Sp | 21 | UUUUGCCUGUCAGCAGCUCAU | AUGAGCUGCUGACAGGCAAAA | [1716-1736](21/21) | [1582-1602](21/21) |
| 70 | Cross Sp | 21 | UGUUUAUGGUCAGCCUCGGAC | GUCCGAGGCUGACCAUAAACA | [1076-1096](21/21) | [942-962](21/21) |
| 71 | Cross Sp | 21 | AUUGUUUAUGGUCAGCCUCGG | CCGAGGCUGACCAUAAACAAU | [1074-1094](21/21) | [940-960](21/21) |
| 72 | Cross Sp | 21 | UGCCAUUGUUUAUGGUCAGCC | GGCUGACCAUAAACAAUGGCA | [1070-1090](21/21) | [936-956](21/21) |
| 73 | Cross Sp | 21 | CAUUCCUGCUCUUGUUGGCAA | UUGCCAACAAGAGCAGGAAUG | [923-943](21/21) | [789-809](21/21) |
| 74 | Cross Sp | 21 | AACAUUCCUGCUCUUGUUGGC | GCCAACAAGAGCAGGAAUGUU | [921-941](21/21) | [787-807](21/21) |
| 75 | Cross Sp | 21 | AUUCAAUGAACAUUCCUGCUC | GAGCAGGAAUGUUCAUUGAAU | [913-933](21/21) | [779-799](21/21) |
| 76 | Cross Sp | 21 | GCCUGUCAGCAGCUCAUACCA | UGGUAUGAGCUGCUGACAGGC | [1720-1740](21/21) | [1586-1606](21/21) |
| 77 | Cross Sp | 21 | UGCCUGUCAGCAGCUCAUACC | GGUAUGAGCUGCUGACAGGCA | [1719-1739](21/21) | [1585-1605](21/21) |
| 78 | Human | 21 | GCAUGGGAUUUGCAACGAAUU | AAUUCGUUGCAAAUCCCAUGC | [895-915](21/21) | [761-781](20/21) |
| 79 | Human | 21 | CUAGGUUAAGACCCUAAUAGA | UCUAUUAGGGUCUUAACCUAG | [7034-7054](21/21) | — |
| 80 | Human | 21 | GGAGACUCUGUCUCAAAAAAA | UUUUUUUGAGACAGAGUCUCC | [6799-6819](21/21) | — |
| 81 | Human | 21 | GGAUGCAAUGAGCCAAGAUCA | UGAUCUUGGCUCAUUGCAUCC | [6748-6768](21/21) | — |
| 82 | Human | 21 | GAAGAACUAGAGGAGCUUGUA | UACAAGCUCCUCUAGUUCUUC | [837-857](21/21) | [703-714](21/21) |
| 83 | Human | 21 | CGAAACCUCAUCUCUACUAAA | UUUAGUAGAGAUGAGGUUUCG | [6632-6652](21/21) | — |
| 84 | Human | 21 | GGAAACCUGGACAAGCAUGAA | UUCAUGCUUGUCCAGGUUUCC | [819-839](21/21) | [685-705](21/21) |
| 85 | Human | 21 | CCAGCAGCCAUCUUAAUACAU | AUGUAUUAAGAUGGCUGCUGG | [6425-6445](21/21) | — |
| 86 | Human | 21 | CAAGACCCCAUCUAAGAAAAA | UUUUUCUUAGAUGGGGUCUUG | [6242-6262](21/21) | — |
| 87 | Human | 21 | GUAGUGGUUUUCCUUCAAACU | AGUUUGAAGGAAAACCACUAC | [5878-5898](21/21) | — |
| 88 | Human | 21 | GGAGCCUGCAUCUAAAUUUCU | AGAAAUUUAGAUGCAGGCUCC | [5433-5453](21/21) | [3569-3580](12/12) |
| 89 | Human | 21 | CUAGCUCCUUUGGCUUUUAAA | UUUAAAAGCCAAAGGAGCUAG | [5378-5398](21/21) | — |
| 90 | Human | 21 | CCAACUCUAGAGAAAACCAAU | AUUGGUUUUCUCUAGAGUUGG | [5254-5274](21/21) | — |
| 91 | Human | 21 | GUAGUCAUCAAGGUGAACAGA | UCUGUUCACCUUGAUGACUAC | [5075-5095](21/21) | — |
| 92 | Human | 21 | GGAUAAGAAAUCCUUACCUUU | AAAGGUAAGGAUUUCUUAUCC | [4920-4940](21/21) | — |
| 93 | Human | 21 | GGAGAGACAGUCUCAUGAUUA | UAAUCAUGAGACUGUCUCUCC | [635-655](21/21) | — |
| 94 | Human | 21 | CGAUUGAUAGAGGUGUGACUA | UAGUCACACCUCUAUCAAUCG | [4653-4673](21/21) | — |
| 95 | Human | 21 | CUACCAAAACUGAUCAUCUGA | UCAGAUGAUCAGUUUUGGUAG | [4630-4650](21/21) | — |
| 96 | Human | 21 | CUAGGUAUCAUCAUUUAUCCA | UGGAUAAAUGAUGAUACCUAG | [4465-4485](21/21) | — |
| 97 | Human | 21 | GCAGGACCUCUGUACUUUCUA | UAGAAAGUACAGAGGUCCUGC | [4360-4380](21/21) | — |
| 98 | Human | 21 | CCACUGGAAUACCACAUAGCU | AGCUAUGUGGUAUUCCAGUGG | [4099-4119](21/21) | — |
| 99 | Human | 21 | CAAGGAGAGAUGCAGAGACUA | UAGUCUCUGCAUCUCUCCUUG | [3963-3983](21/21) | — |
| 100 | Human | 21 | GGACUUUGUGUCAUUGUAUCA | UGAUACAAUGACACAAAGUCC | [515-535](21/21) | [381-401](20/21) |
| 101 | Human | 21 | GGAAAAACAGUGUAUGAAAGU | ACUUUCAUACACUGUUUUUCC | [3358-3378](21/21) | — |
| 102 | Human | 21 | CAAGUAUUACAGGAGAAGGAU | AUCCUUCUCCUGUAAUACUUG | [3325-3345](21/21) | — |
| 103 | Human | 21 | CAACAGAAAGAGAAGAACAAA | UUUGUUCUUCUCUUUCUGUUG | [493-513](21/21) | [359-379](21/21) |
| 104 | Human | 21 | GUACUCACCCUCUUUGGAUAU | AUAUCCAAAGAGGGUGAGUAC | [426-446](21/21) | [292-312](21/21) |
| 105 | Human | 21 | GCAAGUGCCUUCCACUUACUU | AAGUAAGUGGAAGGCACUUGC | [2303-2323](21/21) | [2163-2174](12/12) |
| 106 | Human | 21 | CAACCAUUCUACUUGUGAAAU | AUUUCACAAGUAGAAUGGUUG | [2142-2162](21/21) | [2012-2027](16/16) |
| 107 | Human | 21 | CGAAGGUGACUCACUUUGCUU | AAGCAAAGUGAGUCACCUUCG | [2090-2110](21/21) | — |
| 108 | Human | 21 | CCACUCUAAAUGACAUUUUGU | ACAAAAUGUCAUUUAGAGUGG | [2015-2035](21/21) | — |
| 109 | Human | 21 | CCACCUCCACUCUAAAUGACA | UGUCAUUUAGAGUGGAGGUGG | [2009-2029](21/21) | — |
| 110 | Human | 21 | CCAUCUCACGUGAAAGACAUU | AAUGUCUUUCACGUGAGAUGG | [1968-1988](21/21) | [1842-1853](12/12) |
| 111 | Human | 21 | GCACCAGCCUUGGUAAAGAGU | ACUCUUUACCAAGGCUGGUGC | [1480-1500](21/21) | — |
| 112 | Human | 21 | GCAGAUCAUCACCUCCAUGAA | UUCAUGGAGGUGAUGAUCUGC | [1439-1459](21/21) | [1306-1325](19/20) |
| 113 | Human | 21 | CUACCUGCGAACACAUUCUCA | UGAGAAUGUGUUCGCAGGUAG | [1376-1396](21/21) | [1242-1262](20/21) |
| 114 | Human | 21 | GUGAUUGCCCUCAAGAAGAAA | UUUCUUCUUGAGGGCAAUCAC | [1176-1196](21/21) | [1042-1062](20/21) |
| 115 | Human | 21 | CUGAAGUGAUUGCCCUCAAGA | UCUUGAGGGCAAUCACUUCAG | [1171-1191](21/21) | [1037-1057](20/21) |
| 116 | Human | 21 | CUCAUCCUUGUGGAAGGAAUA | UAUUCCUUCCACAAGGAUGAG | [1119-1139](21/21) | [988-1001](14/14) |
| 117 | Human | 21 | GGAAGAAAAUUCUCAUCCUUG | CAAGGAUGAGAAUUUUCUUCC | [1108-1128](21/21) | — |
| 118 | Human | 21 | CGAAGGCCCUGGAAGAAAAUU | AAUUUUCUUCCAGGGCCUUCG | [1098-1118](21/21) | [965-983](18/19) |
| 119 | Human | 21 | CUAGAGAAGCUAUUGAAAGAU | AUCUUUCAAUAGCUUCUCUAG | [1050-1070](21/21) | — |
| 120 | Human | 21 | CAAUAUGCAAAGCCUAGAGAA | UUCUCUAGGCUUUGCAUAUUG | [1037-1057](21/21) | [903-923](20/21) |
| 121 | Human | 21 | GUCAGGAGCAACCAUUAGAAU | AUUCUAAUGGUUGCUCCUGAC | [1004-1024](21/21) | [870-890](20/21) |
| 122 | Human | 21 | GACUGUCAGGAGCAACCAUUA | UAAUGGUUGCUCCUGACAGUC | [1000-1020](21/21) | [866-885](20/20) |
| 123 | Human | 21 | UGAUUCUGAGUGAUGAACUGA | UCAGUUCAUCACUCAGAAUCA | [952-972](21/21) | [818-838](20/21) |
| 124 | Human | 21 | CUGCUCUUGUUGGCAAAGGUU | AACCUUUGCCAACAAGAGCAG | [928-948](21/21) | [794-814](21/21) |
| 125 | Human | 21 | GGGAUUUGCAACGAAUUCAAU | AUUGAAUUCGUUGCAAAUCCC | [899-919](21/21) | [765-785](20/21) |
| 126 | Human | 21 | CUGACCCUAAAGAAUGCAAAA | UUUUGCAUUCUUUAGGGUCAG | [7216-7236](21/21) | — |
| 127 | Human | 21 | UGAUUCUUUCUGGUAGGAUAA | UUAUCCUACCAGAAAGAAUCA | [7184-7204](21/21) | — |
| 128 | Human | 21 | CCUUAAACUGUGAUUCUUUCU | AGAAAGAAUCACAGUUUAAGG | [7174-7194](21/21) | — |
| 129 | Human | 21 | GACAAGGUUUUGUUUACUACA | UGUAGUAAACAAAACCUUGUC | [7134-7154](21/21) | — |
| 130 | Human | 21 | GCUGGGGACAAGGUUUUGUUU | AAACAAAACCUUGUCCCCAGC | [7128-7148](21/21) | — |
| 131 | Human | 21 | GGCUAGGUUAAGACCCUAAUA | UAUUAGGGUCUUAACCUAGCC | [7032-7052](21/21) | — |
| 132 | Human | 21 | GCAGCUCUUUUCUAGGGAGGA | UCCUCCCUAGAAAAGAGCUGC | [7002-7022](21/21) | — |
| 133 | Human | 21 | CUGUUUUCUAAGCAGCUCUUU | AAAGAGCUGCUUAGAAAACAG | [6991-7011](21/21) | — |
| 134 | Human | 21 | UCAUUUCCAGUGACAUCUGUU | AACAGAUGUCACUGGAAAUGA | [6975-6995](21/21) | — |
| 135 | Human | 21 | AUACUGUUGUUCCUUUUGUCA | UGACAAAAGGAACAACAGUAU | [6957-6977](21/21) | — |
| 136 | Human | 21 | CUAGCCAAUGAUCCCUUUGCU | AGCAAAGGGAUCAUUGGCUAG | [6921-6941](21/21) | — |
| 137 | Human | 21 | CAAGGUUCUUAGGAGUAGAAG | CUUCUACUCCUAAGAACCUUG | [859-879](21/21) | [725-737](13/13) |
| 138 | Human | 21 | CUUCCCUAUCACACAGAAUAA | UUAUUCUGUGUGAUAGGGAAG | [6891-6911](21/21) | — |
| 139 | Human | 21 | GGCUUCCCUAUCACACAGAAU | AUUCUGUGUGAUAGGGAAGCC | [6889-6909](21/21) | — |
| 140 | Human | 21 | GUAGCAAGGUUCUUAGGAGUA | UACUCCUAAGAACCUUGCUAC | [855-875](21/21) | [721-737](17/17) |
| 141 | Human | 21 | CCAGCCAGAGAGCCAAAUUAG | CUAAUUUGGCUCUCUGGCUGG | [6862-6882](21/21) | — |
| 142 | Human | 21 | GGAGCUUGUAGCAAGGUUCUU | AAGAACCUUGCUACAAGCUCC | [848-868](21/21) | [721-734](14/14) |
| 143 | Human | 21 | CCAAGAUCACACCACUGCACU | AGUGCAGUGGUGUGAUCUUGG | [6760-6780](21/21) | — |
| 144 | Human | 21 | CAAGAGGCAGAGGAUGCAAUG | CAUUGCAUCCUCUGCCUCUUG | [6737-6757](21/21) | — |
| 145 | Human | 21 | GAACUAGAGGAGCUUGUAGCA | UGCUACAAGCUCCUCUAGUUC | [840-860](21/21) | — |
| 146 | Human | 21 | GCUGAGGCACAGGAAUUGCUU | AAGCAAUUCCUGUGCCUCAGC | [6711-6731](21/21) | — |
| 147 | Human | 21 | ACAUGGCGAAACCUCAUCUCU | AGAGAUGAGGUUUCGCCAUGU | [6626-6646](21/21) | — |
| 148 | Human | 21 | CAACAUGGCGAAACCUCAUCU | AGAUGAGGUUUCGCCAUGUUG | [6624-6644](21/21) | — |
| 149 | Human | 21 | UCACCUGUAAUCCCAACACUU | AAGUGUUGGGAUUACAGGUGA | [6548-6568](21/21) | — |
| 150 | Human | 21 | CUCAACUUUAUCCACAUGAAA | UUUCAUGUGGAUAAAGUUGAG | [6500-6520](21/21) | — |
| 151 | Human | 21 | GUGGAGAAAAACAUGUCUUUU | AAAAGACAUGUUUUUCUCCAC | [6473-6493](21/21) | — |
| 152 | Human | 21 | GCAGCCAUCUUAAUACAUUAA | UUAAUGUAUUAAGAUGGCUGC | [6428-6448](21/21) | — |
| 153 | Human | 21 | CAGUCCCAGAACCUACAGAUA | UAUCUGUAGGUUCUGGGACUG | [6371-6391](21/21) | — |
| 154 | Human | 21 | CCAAGAACUUACAGGGAUUUU | AAAAUCCCUGUAAGUUCUUGG | [6344-6364](21/21) | — |
| 155 | Human | 21 | GUACUCGGCAGGAAAUUGGAA | UUCCAAUUUCCUGCCGAGUAC | [802-822](21/21) | [670-688](18/19) |
| 156 | Human | 21 | CUACUAAGCCAAGAACUUACA | UGUAAGUUCUUGGCUUAGUAG | [6336-6356](21/21) | — |
| 157 | Human | 21 | GUGAAUGGGGAGGUAUAAAAA | UUUUUAUACCUCCCCAUUCAC | [6297-6317](21/21) | — |
| 158 | Human | 21 | GCAAGACCCCAUCUAAGAAAA | UUUUCUUAGAUGGGGUCUUGC | [6241-6261](21/21) | — |
| 159 | Human | 21 | ACACAGCAAGACCCCAUCUAA | UUAGAUGGGGUCUUGCUGUGU | [6236-6256](21/21) | — |
| 160 | Human | 21 | CAACACAGCAAGACCCCAUCU | AGAUGGGGUCUUGCUGUGUUG | [6234-6254](21/21) | — |
| 161 | Human | 21 | GGAUUGCUUAAACCCAAGAAU | AUUCUUGGGUUUAAGCAAUCC | [6197-6217](21/21) | — |
| 162 | Human | 21 | AAAGUCCUUGAGGAGUAUGGA | UCCAUACUCCUCAAGGACUUU | [768-788](21/21) | [635-654](18/20) |
| 163 | Human | 21 | CCCUGUCUCUACAAAAAUUUU | AAAAUUUUUGUAGAGACAGGG | [5950-5970](21/21) | — |
| 164 | Human | 21 | AGACCCUGUCUCUACAAAAAU | AUUUUUGUAGAGACAGGGUCU | [5947-5967](21/21) | — |
| 165 | Human | 21 | AUAGACCCUGUCUCUACAAAA | UUUUGUAGAGACAGGGUCUAU | [5945-5965](21/21) | — |
| 166 | Human | 21 | AGAUAGACCCUGUCUCUACAA | UUGUAGAGACAGGGUCUAUCU | [5943-5963](21/21) | — |
| 167 | Human | 21 | GCAAGAUAGACCCUGUCUCUA | UAGAGACAGGGUCUAUCUUGC | [5940-5960](21/21) | — |
| 168 | Human | 21 | CUGUCCACAUUCAUGAAGAUU | AAUCUUCAUGAAUGUGGACAG | [5897-5917](21/21) | — |
| 169 | Human | 21 | CAAACUGUCCACAUUCAUGAA | UUCAUGAAUGUGGACAGUUUG | [5893-5913](21/21) | — |
| 170 | Human | 21 | CUUCAAACUGUCCACAUUCAU | AUGAAUGUGGACAGUUUGAAG | [5890-5910](21/21) | — |
| 171 | Human | 21 | GUGAGAGUAGUGGUUUUCCUU | AAGGAAAACCACUACUCUCAC | [5872-5892](21/21) | — |
| 172 | Human | 21 | UUAGAAAAAGAGGUGAGAGUA | UACUCUCACCUCUUUUUCUAA | [5860-5880](21/21) | — |
| 173 | Human | 21 | UAAGCCAAUGAGCUUUAAUCU | AGAUUAAAGCUCAUUGGCUUA | [5818-5838](21/21) | — |
| 174 | Human | 21 | CCUCUCUUGGUGUUCUAGGUA | UACCUAGAACACCAAGAGAGG | [5799-5819](21/21) | — |
| 175 | Human | 21 | CCAAGGUAUCUUUCCUCUCUU | AAGAGAGGAAAGAUACCUUGG | [5786-5806](21/21) | — |
| 176 | Human | 21 | GGUUAUGGGCAGUUGUUGUUU | AAACAACAACUGCCCAUAACC | [5742-5762](21/21) | — |

Table K below shows further siRNA compounds specific for the SSAT gene that have been selected according the present invention.

TABLE K

| | | | | | |
|---|---|---|---|---|---|
| | | Oligo | | | |
| No | Source | Length | Sense siRNA | AntiSense siRNA | human 4506788 |

FOR 19
STRETCHES:

| | | | | | |
|---|---|---|---|---|---|
| 1 | Cross Sp | 19 | CCAUUCCAAAGCUUUAUUA | UAAUAAAGCUUUGGAAUGG | [782-800](19/19) |
| 2 | Cross Sp | 19 | GCAAAGGGAAGAAAAGCAA | UUGCUUUUCUUCCCUUUGC | [138-156](19/19) |
| 3 | Cross Sp | 19 | GUUGUUGCAUGUUUGAAAU | AUUUCAAACAUGCAACAAC | [809-827](19/19) |
| 4 | Cross Sp | 19 | CCAUGUACUAUUUUACCUA | UAGGUAAAAUAGUACAUGG | [389-407](19/19) |
| 5 | Cross Sp | 19 | AGACGAAAAUGGCUAAAUU | AAUUUAGCCAUUUUCGUCU | [158-176](19/19) |
| 6 | Cross Sp | 19 | GGUACUUAGAGUUUCUGUU | AACAGAAACUCUAAGUACC | [983-1001](19/19) |
| 7 | Cross Sp | 19 | CACACUGGUACUUAGAGUU | AACUCUAAGUACCAGUGUG | [977-995](19/19) |
| 8 | Cross Sp | 19 | GUACACACUGGUACUUAGA | UCUAAGUACCAGUGUGUAC | [974-992](19/19) |
| 9 | Cross Sp | 19 | GUACAAUGUACACACUGGU | ACCAGUGUGUACAUUGUAC | [967-985](19/19) |
| 10 | Cross Sp | 19 | GUGAGUCAUUUAAAUGUGU | ACACAUUUAAAUGACUCAC | [950-968](19/19) |
| 11 | Cross Sp | 19 | CGUUGUUGCAUGUUUGAAA | UUUCAAACAUGCAACAACG | [808-826](19/19) |
| 12 | Cross Sp | 19 | CCAAAGCUUUAUUACCAGU | ACUGGUAAUAAAGCUUUGG | [787-805](19/19) |
| 13 | Cross Sp | 19 | GAAGAAUCUAAGCCAGGUU | AACCUGGCUUAGAUUCUUC | [495-513](19/19) |
| 14 | Cross Sp | 19 | GUACUAUUUUACCUAUGAC | GUCAUAGGUAAAAUAGUAC | [393-411](19/19) |
| 15 | Cross Sp | 19 | GCCAUGUACUAUUUUACCU | AGGUAAAAUAGUACAUGGC | [388-406](19/19) |
| 16 | Cross Sp | 19 | GGCUAAAUAUGAAUACAUG | CAUGUAUUCAUAUUUAGCC | [237-255](19/19) |
| 17 | Cross Sp | 19 | GAAAAGCAAAAGACGAAAA | UUUUCGUCUUUUGCUUUUC | [148-166](19/19) |
| 18 | Cross Sp | 19 | GGAAGAAAAGCAAAAGACG | CGUCUUUUGCUUUUCUUCC | [144-162](19/19) |
| 19 | Cross Sp | 19 | GGGAAGAAAAGCAAAAGAC | GUCUUUUGCUUUUCUUCCC | [143-161](19/19) |
| 20 | Cross Sp | 19 | AAAGGGAAGAAAAGCAAAA | UUUUGCUUUUCUUCCCUUU | [140-158](19/19) |
| 21 | Cross Sp | 19 | CGCAAAGGGAAGAAAAGCA | UGCUUUUCUUCCCUUUGCG | [137-155](19/19) |
| 22 | Cross Sp | 19 | GAAUGAGGAACCACCUCCU | AGGAGGUGGUUCCUCAUUC | [90-108](19/19) |
| 23 | Cross Sp | 19 | CUACUUAGAGUUUCUGUUU | AAAGAGAAACUCUAAGUAC | [984-1002](19/19) |
| 24 | Cross Sp | 19 | ACACACUGGUACUUAGAGU | ACUCUAAGUACCAGUGUGU | [976-994](19/19) |
| 25 | Cross Sp | 19 | ACAAUGUACACACUGGUAC | GUACCAGUGUGUACAUUGU | [969-987](19/19) |
| 26 | Cross Sp | 19 | UUCCAAAGCUUUAUUACCA | UGGUAAUAAAGCUUUGGAA | [785-803](19/19) |
| 27 | Cross Sp | 19 | GAUCUGUCCAGUGAAGAGG | CCUCUUCACUGGACAGAUC | [604-622](19/19) |
| 28 | Cross Sp | 19 | UGCACUUCUUGGUAGCAGA | UCUGCUACCAAGAAGUGCA | [539-557](19/19) |
| 29 | Cross Sp | 19 | AAGAAUCUAAGCCAGGUUG | CAACCUGGCUUAGAUUCUU | [496-514](19/19) |
| 30 | Cross Sp | 19 | UACUAUUUUACCUAUGACC | GGUCAUAGGUAAAAUAGUA | [394-412](19/19) |
| 31 | Cross Sp | 19 | ACUGCCUGGUUGCAGAAGU | ACUUCUGCAACCAGGCAGU | [323-341](19/19) |
| 32 | Cross Sp | 19 | CUGGCUAAAUAUGAAUACA | UGUAUUCAUAUUUAGCCAG | [235-253](19/19) |
| 33 | Cross Sp | 19 | AAAGACGAAAAUGGCUAAA | UUUAGCCAUUUUCGUCUUU | [156-174](19/19) |
| 34 | Cross Sp | 19 | CAAAAGACGAAAAUGGCUA | UAGCCAUUUUCGUCUUUUG | [154-172](19/19) |
| 35 | Cross Sp | 19 | GCAAAAGACGAAAAUGGCU | AGCCAUUUUCGUCUUUUGC | [153-171](19/19) |
| 36 | Cross Sp | 19 | AAGCAAAAGACGAAAAUGG | CCAUUUUCGUCUUUUGCUU | [151-169](19/19) |
| 37 | Cross Sp | 19 | AGAAAAGCAAAAGACGAAA | UUUCGUCUUUUGCUUUUCU | [147-165](19/19) |
| 38 | Cross Sp | 19 | GAAGAAAAGCAAAAGACGA | UCGUCUUUUGCUUUUCUUC | [145-163](19/19) |
| 39 | Cross Sp | 19 | AGGGAAGAAAAGCAAAAGA | UCUUUUGCUUUUCUUCCCU | [142-160](19/19) |
| 40 | Cross Sp | 19 | CAAAGGGAAGAAAAGCAAA | UUUGCUUUUCUUCCCUUUG | [139-157](19/19) |
| 41 | Cross Sp | 19 | UGACUGAGAAGAGGACGCU | AGCGUCCUCUUCUCAGUCA | [61-79](19/19) |
| 42 | Cross Sp | 19 | CUGGUACUUAGAGUUUCUG | CAGAAACUCUAAGUACCAG | [981-999](19/19) |
| 43 | Cross Sp | 19 | ACUGGUACUUAGAGUUUCU | AGAAACUCUAAGUACCAGU | [980-998](19/19) |
| 44 | Cross Sp | 19 | ACACUGGUACUUAGAGUUU | AAACUCUAAGUACCAGUGU | [978-996](19/19) |
| 45 | Cross Sp | 19 | UGUACACACUGGUACUUAG | CUAAGUACCAGUGUGUACA | [973-991](19/19) |
| 46 | Cross Sp | 19 | AAUGUACACACUGGUACUU | AAGUACCAGUGUGUACAUU | [971-989](19/19) |
| 47 | Cross Sp | 19 | UGUGUACAAUGUACACACU | AGUGUGUACAUUGUACACA | [964-982](19/19) |
| 48 | Cross Sp | 19 | GAGUCAUUUAAAUGUGUAC | GUACACAUUUAAAUGACUC | [952-970](19/19) |
| 49 | Cross Sp | 19 | CAAAGCUUUAUUACCAGUG | CACUGGUAAUAAAGCUUUG | [788-806](19/19) |
| 50 | Cross Sp | 19 | UCCAAAGCUUUAUUACCAG | CUGGUAAUAAAGCUUUGGA | [786-804](19/19) |
| 51 | Cross Sp | 19 | CAUUCCAAAGCUUUAUUAC | GUAAUAAAGCUUUGGAAUG | [783-801](19/19) |
| 52 | Cross Sp | 19 | AGAAUCUAAGCCAGGUUGC | GCAACCUGGCUUAGAUUCU | [497-515](19/19) |
| 53 | Cross Sp | 19 | UGAAGAAUCUAAGCCAGGU | ACCUGGCUUAGAUUCUUCA | [494-512](19/19) |
| 54 | Cross Sp | 19 | GCUAAAUAUGAAUACAUGG | CCAUGUAUUCAUAUUUAGC | [238-256](19/19) |
| 55 | Cross Sp | 19 | AAGACGAAAAUGGCUAAAU | AUUUAGCCAUUUUCGUCUU | [157-175](19/19) |
| 56 | Cross Sp | 19 | AAAAGACGAAAAUGGCUAA | UUAGCCAUUUUCGUCUUUU | [155-173](19/19) |
| 57 | Cross Sp | 19 | AGCAAAAGACGAAAAUGGC | GCCAUUUUCGUCUUUUGCU | [152-170](19/19) |
| 58 | Cross Sp | 19 | AAGAAAAGCAAAAGACGAA | UUCGUCUUUUGCUUUUCUU | [146-164](19/19) |
| 59 | Cross Sp | 19 | UGUUCAAGUACAGGGGCCU | AGGCCCCUGUACUUGAACA | [114-132](19/19) |
| 60 | Cross Sp | 19 | UGGUACUUAGAGUUUCUGU | ACAGAAACUCUAAGUACCA | [982-1000](19/19) |
| 61 | Cross Sp | 19 | CACUGGUACUUAGAGUUUC | GAAACUCUAAGUACCAGUG | [979-997](19/19) |
| 62 | Cross Sp | 19 | CAAUGUACACACUGGUACU | AGUACCAGUGUGUACAUUG | [970-988](19/19) |
| 63 | Cross Sp | 19 | GUGUACAAUGUACACACUG | CAGUGUGUACAUUGUACAC | [965-983](19/19) |
| 64 | Cross Sp | 19 | AUGUGUACAAUGUACACAC | GUGUGUACAUUGUACACAU | [963-981](19/19) |
| 65 | Cross Sp | 19 | AAUGUGUACAAUGUACACA | UGUGUACAUUGUACACAUU | [962-980](19/19) |
| 66 | Cross Sp | 19 | AUUCCAAAGCUUUAUUACC | GGUAAUAAAGCUUUGGAAU | [784-802](19/19) |
| 67 | Cross Sp | 19 | AUGCACUUCUUGGUAGCAG | CUGCUACCAAGAAGUGCAU | [538-556](19/19) |
| 68 | Cross Sp | 19 | ACUAUUUUACCUAUGACCC | GGGUCAUAGGUAAAAUAGU | [395-413](19/19) |
| 69 | Cross Sp | 19 | AAAGCAAAAGACGAAAAUG | CAUUUUCGUCUUUUGCUUU | [150-168](19/19) |
| 70 | Cross Sp | 19 | AAGGGAAGAAAAGCAAAAG | CUUUUGCUUUUCUUCCCUU | [141-159](19/19) |
| 71 | Cross Sp | 19 | AAUGAGGAACCACCUCCUC | GAGGAGGUGGUUCCUCAUU | [91-109](19/19) |
| 72 | Cross Sp | 19 | AGCCUGACUGAGAAGAGGA | UCCUCUUCUCAGUCAGGCU | [57-75](19/19) |
| 73 | Cross Sp | 19 | UACACACUGGUACUUAGAG | CUCUAAGUACCAGUGUGUA | [975-993](19/19) |
| 74 | Cross Sp | 19 | AUGUACACACUGGUACUUA | UAAGUACCAGUGUGUACAU | [972-990](19/19) |
| 75 | Cross Sp | 19 | UACAAUGUACACACUGGUA | UACCAGUGUGUACAUUGUA | [968-986](19/19) |
| 76 | Cross Sp | 19 | UGUACAAUGUACACACUGG | CCAGUGUGUACAUUGUACA | [966-984](19/19) |
| 77 | Cross Sp | 19 | AAAUGUGUACAAUGUACAC | GUGUACAUUGUACACAUUU | [961-979](19/19) |
| 78 | Cross Sp | 19 | AUCUGUCCAGUGAAGAGGG | CCCUCUUCACUGGACAGAU | [605-623](19/19) |
| 79 | Cross Sp | 19 | ACGAAUGAGGAACCACCUC | GAGGUGGUUCCUCAUUCGU | [88-106](19/19) |
| 80 | Cross Sp | 19 | GUGCCAGCCUGACUGAGAA | UUCUCAGUCAGGCUGGCAC | [52-70](19/19) |
| 81 | Cross Sp | 19 | CUGCCUGGUUGCAGAAGUG | CACUUCUGCAACCAGGCAG | [324-342](19/19) |
| 82 | Cross Sp | 19 | CCACUGCCUGGUUGCAGAA | UUCUGCAACCAGGCAGUGG | [321-339](19/19) |
| 83 | Cross Sp | 19 | UGAGGAACCACCUCCUCCU | AGGAGGAGGUGGUUCCUCA | [93-111](19/19) |
| 84 | Cross Sp | 19 | GCCAGCCUGACUGAGAAGA | UCUUCUCAGUCAGGCUGGC | [54-72](19/19) |
| 85 | Cross Sp | 19 | ACCACUGCCUGGUUGCAGA | UCUGCAACCAGGCAGUGGU | [320-338](19/19) |
| 86 | Cross Sp | 19 | CGAAUGAGGAACCACCUCC | GGAGGUGGUUCCUCAUUCG | [89-107](19/19) |
| 87 | Cross Sp | 19 | GCCUGACUGAGAAGAGGAC | GUCCUCUUCUCAGUCAGGC | [58-76](19/19) |
| 88 | Cross Sp | 19 | CAGCCUGACUGAGAAGAGG | CCUCUUCUCAGUCAGGCUG | [56-74](19/19) |
| 89 | Cross Sp | 19 | CCAGCCUGACUGAGAAGAG | CUCUUCUCAGUCAGGCUGG | [55-73](19/19) |
| 90 | Cross Sp | 19 | AUGGUGCCAGCCUGACUGA | UCAGUCAGGCUGGCACCAU | [49-67](19/19) |
| 91 | Cross Sp | 19 | UCAAGUACAGGGGCCUGGU | ACCAGGCCCCUGUACUUGA | [117-135](19/19) |
| 92 | Cross Sp | 19 | CUGUUCAAGUACAGGGGCC | GGCCCCUGUACUUGAACAG | [113-131](19/19) |
| 93 | Cross Sp | 19 | UCAUGGUGCCAGCCUGACU | AGUCAGGCUGGCACCAUGA | [47-65](19/19) |
| 94 | Cross Sp | 19 | ACUGAGAAGAGGACGCUCC | GGAGCGUCCUCUUCUCAGU | [63-81](19/19) |
| 95 | Cross Sp | 19 | CUGACUGAGAAGAGGACGC | GCGUCCUCUUCUCAGUCAG | [60-78](19/19) |
| 96 | Cross Sp | 19 | UGCCAGCCUGACUGAGAAG | CUUCUCAGUCAGGCUGGCA | [53-71](19/19) |
| 97 | Cross Sp | 19 | UGCCUGGUUGCAGAAGUGC | GCACUUCUGCAACCAGGCA | [325-343](19/19) |
| 98 | Cross Sp | 19 | CACUGCCUGGUUGCAGAAG | CUUCUGCAACCAGGCAGUG | [322-340](19/19) |
| 99 | Cross Sp | 19 | UUCAAGUACAGGGGCCUGG | CCAGGCCCCUGUACUUGAA | [116-134](19/19) |
| 100 | Cross Sp | 19 | UACCACUGCCUGGUUGCAG | CUGCAACCAGGCAGUGGUA | [319-337](19/19) |
| 101 | Human | 19 | GGAAGAACAAGUAAUCUUA | UAAGAUUACUUGUUCUUCC | [255-273](19/19) |
| 102 | Human | 19 | GCAGCAGCAUGCACUUCUU | AAGAAGUGCAUGCUGCUGC | [530-548](19/19) |
| 103 | Human | 19 | GCAAGUUAUUGUAUCUUGA | UCAAGAUACAAUAACUUGC | [422-440](19/19) |
| 104 | Human | 19 | GGAUUGGCAAGUUAUUGUA | UACAAUAACUUGCCAAUCC | [416-434](19/19) |
| 105 | Human | 19 | AUACAUGGAAGAACAAGUA | UACUUGUUCUUCCAUGUAU | [249-267](19/19) |
| 106 | Human | 19 | GUGGUGUGAUCUUAAUAUA | UAUAUUAAGAUCACACCAC | [910-928](19/19) |
| 107 | Human | 19 | CGAUAAACAACAAGGUGGU | ACCACCUUGUUGUUUAUCG | [896-914](19/19) |
| 108 | Human | 19 | CCUUGAAUAUCUUUCGAUA | UAUCGAAAGAUAUUCAAGG | [882-900](19/19) |
| 109 | Human | 19 | AGAUCUUUCUCCUUGAAUA | UAUUCAAGGAGAAAGAUCU | [872-890](19/19) |
| 110 | Human | 19 | UCAGAUCUUUCUCCUUGAA | UUCAAGGAGAAAGAUCUGA | [870-888](19/19) |
| 111 | Human | 19 | GCAGUUUGGAGAGUCAGAU | AUCUGACUCUCCAAACUGC | [857-875](19/19) |
| 112 | Human | 19 | GGAGCUGGCUAAAUAUGAA | UUCAUAUUUAGCCAGCUCC | [231-249](19/19) |
| 113 | Human | 19 | CAAGGAGCUGGCUAAAUAU | AUAUUUAGCCAGCUCCUUG | [228-246](19/19) |
| 114 | Human | 19 | UCAAGGAGCUGGCUAAAUA | UAUUUAGCCAGCUCCUUGA | [227-245](19/19) |
| 115 | Human | 19 | GCACCCAUUCCAAAGCUUU | AAAGCUUUGGAAUGGGUGC | [778-796](19/19) |
| 116 | Human | 19 | AGAAUGAGCACCCAUUCCA | UGGAAUGGGUGCUCAUUCU | [771-789](19/19) |
| 117 | Human | 19 | CAACUUCUCUUGCUUUCUA | UAGAAAGCAAGAGAAGUUG | [731-749](19/19) |
| 118 | Human | 19 | CAACCUCCAUUCUAUUUUA | UAAAAUAGAAUGGAGGUUG | [701-719](19/19) |
| 119 | Human | 19 | GUAGAUGACAACCUCCAUU | AAUGGAGGUUGUCAUCUAC | [693-711](19/19) |
| 120 | Human | 19 | ACAAGGAGUACUUGCUAAA | UUUAGCAAGUACUCCUUGU | [644-662](19/19) |
| 121 | Human | 19 | AGAUCGACAAGGAGUACUU | AAGUACUCCUUGUCGAUCU | [638-656](19/19) |
| 122 | Human | 19 | GGAGACUGUUCAAGAUCGA | UCGAUCUUGAACAGUCUCC | [626-644](19/19) |
| 123 | Human | 19 | GUUGGAGACUGUUCAAGAU | AUCUUGAACAGUCUCCAAC | [623-641](19/19) |
| 124 | Human | 19 | GAGGGUUGGAGACUGUUCA | UGAACAGUCUCCAACCCUC | [619-637](19/19) |
| 125 | Human | 19 | GAAGAGGGUUGGAGACUGU | ACAGUCUCCAACCCUCUUC | [616-634](19/19) |
| 126 | Human | 19 | CCAUCAACUUCUAUAAAAG | CUUUUAUAGAAGUUGAUGG | [572-590](19/19) |
| 127 | Human | 19 | CAUCCAUCAACUUCUAUAA | UUAUAGAAGUUGAUGGAUG | [569-587](19/19) |
| 128 | Human | 19 | CCAUCCAUCAACUUCUAUA | UAUAGAAGUUGAUGGAUGG | [568-586](19/19) |
| 129 | Human | 19 | GAACCAUCCAUCAACUUCU | AGAAGUUGAUGGAUGGUUC | [565-583](19/19) |
| 130 | Human | 19 | GUAGCAGAAUGGAAUGAAC | GUUCAUUCCAUUCUGCUAC | [550-568](19/19) |
| 131 | Human | 19 | CUUGGUAGCAGAAUGGAAU | AUUCCAUUCUGCUACCAAG | [546-564](19/19) |
| 132 | Human | 19 | GCAGCAUGCACUUCUUGGU | ACCAAGAAGUGCAUGCUGC | [533-551](19/19) |
| 133 | Human | 19 | CGACUGCAGUGACAUACUG | CAGUAUGUCACUGCAGUCG | [201-219](19/19) |
| 134 | Human | 19 | CAGAAAUUCUGAAGAAUCU | AGAUUCUUCAGAAUUUCUG | [485-503](19/19) |
| 135 | Human | 19 | GGAUCAGAAAUUCUGAAGA | UCUUCAGAAUUUCUGAUCC | [481-499](19/19) |
| 136 | Human | 19 | CGUGAUGAGUGAUUAUAGA | UCUAUAAUCACUCAUCACG | [450-468](19/19) |
| 137 | Human | 19 | CCCGUGGAUUGGCAAGUUA | UAACUUGCCAAUCCACGGG | [411-429](19/19) |
| 138 | Human | 19 | ACACAGCAUUGUUGGUUUU | AAAACCAACAAUGCUGUGU | [369-387](19/19) |
| 139 | Human | 19 | GAAGGACACAGCAUUGUUG | CAACAAUGCUGUGUCCUUC | [364-382](19/19) |
| 140 | Human | 19 | GCUAGAAGAUGGUUUUGGA | UCCAAAACCAUCUUCUAGC | [288-306](19/19) |
| 141 | Human | 19 | GAACAAGUAAUCUUAACUG | CAGUUAAGAUUACUUGUUC | [259-277](19/19) |
| 142 | Human | 19 | CAUGGAAGAACAAGUAAUC | GAUUACUUGUUCUUCCAUG | [252-270](19/19) |
| 143 | Human | 19 | ACAUGGAAGAACAAGUAAU | AUUACUUGUUCUUCCAUGU | [251-269](19/19) |
| 144 | Human | 19 | UGAAUACAUGGAAGAACAA | UUGUUCUUCCAUGUAUUCA | [246-264](19/19) |
| 145 | Human | 19 | AUGAAUACAUGGAAGAACA | UGUUCUUCCAUGUAUUCAU | [245-263](19/19) |
| 146 | Human | 19 | UCUCGUGAGUCAUUUAAAU | AUUUAAAUGACUCACGAGA | [946-964](19/19) |
| 147 | Human | 19 | AUUCUCGUGAGUCAUUUAA | UUAAAUGACUCACGAGAAU | [944-962](19/19) |
| 148 | Human | 19 | UCAUUCUCGUGAGUCAUUU | AAAUGACUCACGAGAAUGA | [942-960](19/19) |
| 149 | Human | 19 | AAACUUCAUUCUCGUGAGU | ACUCACGAGAAUGAAGUUU | [937-955](19/19) |
| 150 | Human | 19 | GGUGGUGUGAUCUUAAUAU | AUAUUAAGAUCACACCACC | [909-927](19/19) |
| 151 | Human | 19 | AAGGUGGUGUGAUCUUAAU | AUUAAGAUCACACCACCUU | [907-925](19/19) |
| 152 | Human | 19 | CAAGGUGGUGUGAUCUUAA | UUAAGAUCACACCACCUUG | [906-924](19/19) |
| 153 | Human | 19 | ACAAGGUGGUGUGAUCUUA | UAAGAUCACACCACCUUGU | [905-923](19/19) |
| 154 | Human | 19 | CAACAAGGUGGUGUGAUCU | AGAUCACACCACCUUGUUG | [903-921](19/19) |
| 155 | Human | 19 | ACAACAAGGUGGUGUGAUC | GAUCACACCACCUUGUUGU | [902-920](19/19) |
| 156 | Human | 19 | CUCCUUGAAUAUCUUUCGA | UCGAAAGAUAUUCAAGGAG | [880-898](19/19) |
| 157 | Human | 19 | CUUUCUCCUUGAAUAUCUU | AAGAUAUUCAAGGAGAAAG | [876-894](19/19) |
| 158 | Human | 19 | GAUCUUUCUCCUUGAAUAU | AUAUUCAAGGAGAAAGAUC | [873-891](19/19) |
| 159 | Human | 19 | AGAGUCAGAUCUUUCUCCU | AGGAGAAAGAUCUGACUCU | [866-884](19/19) |
| 160 | Human | 19 | UGGAGAGUCAGAUCUUUCU | AGAAAGAUCUGACUCUCCA | [863-881](19/19) |
| 161 | Human | 19 | AGUUUGGAGAGUCAGAUCU | AGAUCUGACUCUCCAAACU | [859-877](19/19) |
| 162 | Human | 19 | GAUGCAGUUUGGAGAGUCA | UGACUCUCCAAACUGCAUC | [854-872](19/19) |
| 163 | Human | 19 | CUCAGAUGCAGUUUGGAGA | UCUCCAAACUGCAUCUGAG | [850-868](19/19) |
| 164 | Human | 19 | GGCAAUCUCAGAUGCAGUU | AACUGCAUCUGAGAUUGCC | [844-862](19/19) |
| 165 | Human | 19 | GAGCUGGCUAAAUAUGAAU | AUUCAUAUUUAGCCAGCUC | [232-250](19/19) |
| 166 | Human | 19 | AAUGAGGUCUGUUUAAAGU | ACUUUAAACAGACCUCAUU | [825-843](19/19) |
| 167 | Human | 19 | GAAAUGAGGUCUGUUUAAA | UUUAAACAGACCUCAUUUC | [823-841](19/19) |
| 168 | Human | 19 | UUUGAAAUGAGGUCUGUUU | AAACAGACCUCAUUUCAAA | [820-838](19/19) |
| 169 | Human | 19 | GGCGUUGUUGCAUGUUUGA | UCAAACAUGCAACAACGCC | [806-824](19/19) |
| 170 | Human | 19 | GUGGCGUUGUUGCAUGUUU | AAACAUGCAACAACGCCAC | [804-822](19/19) |
| 171 | Human | 19 | CAGUGGCGUUGUUGCAUGU | ACAUGCAACAACGCCACUG | [802-820](19/19) |
| 172 | Human | 19 | CCCAUUCCAAAGCUUUAUU | AAUAAAGCUUUGGAAUGGG | [781-799](19/19) |
| 173 | Human | 19 | AGCACCCAUUCCAAAGCUU | AAGCUUUGGAAUGGGUGCU | [777-795](19/19) |
| 174 | Human | 19 | GAGCACCCAUUCCAAAGCU | AGCUUUGGAAUGGGUGCUC | [776-794](19/19) |
| 175 | Human | 19 | GAAUGAGCACCCAUUCCAA | UUGGAAUGGGUGCUCAUUC | [772-790](19/19) |
| 176 | Human | 19 | UGAUCAAGGAGCUGGCUAA | UUAGCCAGCUCCUUGAUCA | [224-242](19/19) |
| 177 | Human | 19 | GCUGUUUGUAGUGAAAUAA | UUAUUUCACUACAAACAGC | [751-769](19/19) |
| 178 | Human | 19 | UAUGCUGUUUGUAGUGAAA | UUUCACUACAAACAGCAUA | [748-766](19/19) |
| 179 | Human | 19 | CUAUGCUGUUUGUAGUGAA | UUCACUACAAACAGCAUAG | [747-765](19/19) |
| 180 | Human | 19 | UUUCUAUGCUGUUUGUAGU | ACUACAAACAGCAUAGAAA | [744-762](19/19) |
| 181 | Human | 19 | UGCUUUCUAUGCUGUUUGU | ACAAACAGCAUAGAAAGCA | [741-759](19/19) |
| 182 | Human | 19 | CUCUUGCUUUCUAUGCUGU | ACAGCAUAGAAAGCAAGAG | [737-755](19/19) |
| 183 | Human | 19 | AACUUCUCUUGCUUUCUAU | AUAGAAAGCAAGAGAAGUU | [732-750](19/19) |
| 184 | Human | 19 | CCCAACUUCUCUUGCUUUC | GAAAGCAAGAGAAGUUGGG | [729-747](19/19) |
| 185 | Human | 19 | UUCCCAACUUCUCUUGCUU | AAGCAAGAGAAGUUGGGAA | [727-745](19/19) |
| 186 | Human | 19 | GAAUAAAUUCCCAACUUCU | AGAAGUUGGGAAUUUAUUC | [720-738](19/19) |

FOR 21
STRETCHES:

| | | | | | |
|---|---|---|---|---|---|
| 1 | Cross Sp | 21 | GAAGAAAAGCAAAAGACGAAA | UUUCGUCUUUUGCUUUUCUUC | [145-165](21/21) |
| 2 | Cross Sp | 21 | CGCAAAGGGAAGAAAAGCAAA | UUUGCUUUUCUUCCCUUUGCG | [137-157](21/21) |
| 3 | Cross Sp | 21 | ACACACUGGUACUUAGAGUUU | AAACUCUAAGUACCAGUGUGU | [976-996](21/21) |
| 4 | Cross Sp | 21 | GUACACACUGGUACUUAGAGU | ACUCUAAGUACCAGUGUGUAC | [974-994](21/21) |
| 5 | Cross Sp | 21 | AUGUACACACUGGUACUUAGA | UCUAAGUACCAGUGUGUACAU | [972-992](21/21) |
| 6 | Cross Sp | 21 | CAAUGUACACACUGGUACUUA | UAAGUACCAGUGUGUACAUUG | [970-990](21/21) |
| 7 | Cross Sp | 21 | CCAUUCCAAAGCUUUAUUACC | GGUAAUAAAGCUUUGGAAUGG | [782-802](21/21) |
| 8 | Cross Sp | 21 | GAAGAAUCUAAGCCAGGUUGC | GCAACCUGGCUUAGAUUCUUC | [495-515](21/21) |
| 9 | Cross Sp | 21 | GUACUAUUUUACCUAUGACCC | GGGUCAUAGGUAAAAUAGUAC | [393-413](21/21) |
| 10 | Cross Sp | 21 | CCAUGUACUAUUUUACCUAUG | CAUAGGUAAAAUAGUACAUGG | [389-409](21/21) |
| 11 | Cross Sp | 21 | GCCAUGUACUAUUUUACCUAU | AUAGGUAAAAUAGUACAUGGC | [388-408](21/21) |
| 12 | Cross Sp | 21 | CAAAAGACGAAAAUGGCUAAA | UUUAGCCAUUUUCGUCUUUUG | [154-174](21/21) |
| 13 | Cross Sp | 21 | GCAAAAGACGAAAAUGGCUAA | UUAGCCAUUUUCGUCUUUUGC | [153-173](21/21) |
| 14 | Cross Sp | 21 | AAAGCAAAAGACGAAAAUGGC | GCCAUUUUCGUCUUUUGCUUU | [150-170](21/21) |
| 15 | Cross Sp | 21 | GAAAAGCAAAAGACGAAAAUG | CAUUUUCGUCUUUUGCUUUUC | [148-168](21/21) |
| 16 | Cross Sp | 21 | GCAAAGGGAAGAAAAGCAAAA | UUUUGCUUUUCUUCCCUUUGC | [138-158](21/21) |
| 17 | Cross Sp | 21 | UGGUACUUAGAGUUUCUGUUU | AAACAGAAACUCUAAGUACCA | [982-1002](21/21) |
| 18 | Cross Sp | 21 | ACAAUGUACACACUGGUACUU | AAGUACCAGUGUGUACAUUGU | [969-989](21/21) |
| 19 | Cross Sp | 21 | UACAAUGUACACACUGGUACU | AGUACCAGUGUGUACAUUGUA | [968-988](21/21) |
| 20 | Cross Sp | 21 | GUACAAUGUACACACUGGUAC | GUACCAGUGUGUACAUUGUAC | [967-987](21/21) |
| 21 | Cross Sp | 21 | UGUACAAUGUACACACUGGUA | UACCAGUGUGUACAUUGUACA | [966-986](21/21) |
| 22 | Cross Sp | 21 | GUGUACAAUGUACACACUGGU | ACCAGUGUGUACAUUGUACAC | [965-985](21/21) |
| 23 | Cross Sp | 21 | AUGUGUACAAUGUACACACUG | CAGUGUGUACAUUGUACACAU | [963-983](21/21) |
| 24 | Cross Sp | 21 | GUGAGUCAUUUAAAUGUGUAC | GUACACAUUUAAAUGACUCAC | [950-978](21/21) |
| 25 | Cross Sp | 21 | UGAAGAAUCUAAGCCAGGUUG | CAACCUGGCUUAGAUUCUUCA | [494-514](21/21) |
| 26 | Cross Sp | 21 | UACCACUGCCUGGUUGCAGAA | UUCUGCAACCAGGCAGUGGUA | [494-514](21/21) |
| 27 | Cross Sp | 21 | UGGCUAAAUAUGAAUACAUGG | CCAUGUAUUCAUAUUUAGCCA | [236-256](21/21) |
| 28 | Cross Sp | 21 | CUGGCUAAAUAUGAAUACAUG | CAUGUAUUCAUAUUUAGCCAG | [235-255](21/21) |
| 29 | Cross Sp | 21 | GGAAGAAAAGCAAAAGACGAA | UUCGUCUUUUGCUUUUCUUCC | [144-164](21/21) |
| 30 | Cross Sp | 21 | GGGAAGAAAAGCAAAAGACGA | UCGUCUUUUGCUUUUCUUCCC | [143-163](21/21) |
| 31 | Cross Sp | 21 | AGGGAAGAAAAGCAAAAGACG | CGUCUUUUGCUUUUCUUCCCU | [142-162](21/21) |
| 32 | Cross Sp | 21 | AAAGGGAAGAAAAGCAAAAGA | UCUUUUGCUUUUCUUCCCUUU | [140-160](21/21) |
| 33 | Cross Sp | 21 | CAAAGGGAAGAAAAGCAAAAG | CUUUUGCUUUUCUUCCCUUUG | [139-159](21/21) |
| 34 | Cross Sp | 21 | CUGGUACUUAGAGUUUCUGUU | AACAGAAACUCUAAGUACCAG | [981-1001](21/21) |
| 35 | Cross Sp | 21 | ACUGGUACUUAGAGUUUCUGU | ACAGAAACUCUAAGUACCAGU | [980-1000](21/21) |
| 36 | Cross SP | 21 | CACUGGUACUUAGAGUUUCUG | CAGAAACUCUAAGUACCAGUG | [979-999](21/21) |
| 37 | Cross Sp | 21 | ACACUGGUACUUAGAGUUUCU | AGAAACUCUAAGUACCAGUGU | [978-998](21/21) |
| 38 | Cross Sp | 21 | UACACACUGGUACUUAGAGUU | AACUCUAAGUACCAGUGUGUA | [975-995](21/21) |
| 39 | Cross Sp | 21 | UGUACACACUGGUACUUAGAG | CUCUAAGUACCAGUGUGUACA | [973-993](21/21) |
| 40 | Cross Sp | 21 | AAUGUGUACAAUGUACACACU | AGUGUGUACAUUGUACACAUU | [962-982](21/21) |
| 41 | Cross Sp | 21 | UUCCAAAGCUUUAUUACCAGU | ACUGGUAAUAAAGCUUUGGAA | [785-805](21/21) |
| 42 | Cross Sp | 21 | AUUCCAAAGCUUUAUUACCAG | CUGGUAAUAAAGCUUUGGAAU | [784-804](21/21) |
| 43 | Cross Sp | 21 | CAUUCCAAAGCUUUAUUACCA | UGGUAAUAAAGCUUUGGAAUG | [783-803](21/21) |
| 44 | Cross Sp | 21 | UGUACUAUUUUACCUAUGACC | GGUCAUAGGUAAAAUAGUACA | [392-412](21/21) |
| 45 | Cross Sp | 21 | GGCUAAAUAUGAAUACAUGGA | UCCAUGUAUUCAUAUUUAGCC | [237-257](21/21) |
| 46 | Cross Sp | 21 | AAGCAAAAGACGAAAAUGGCU | AGCCAUUUUCGUCUUUUGCUU | [151-171](21/21) |
| 47 | Cross Sp | 21 | AAGGGAAGAAAAGCAAAAGAC | GUCUUUUGCUUUUCUUCCCUU | [141-161](21/21) |
| 48 | Cross Sp | 21 | ACGAAUGAGGAACCACCUCCU | AGGAGGUGGUUCCUCAUUCGU | [88-108](21/21) |
| 49 | Cross Sp | 21 | CACACUGGUACUUAGAGUUUC | GAAACUCUAAGUACCAGUGUG | [977-997](21/21) |
| 50 | Cross Sp | 21 | AAAUGUGUACAAUGUACACAC | GUGUGUACAUUGUACACAUUU | [961-981](21/21) |
| 51 | Cross Sp | 21 | UCCAAAGCUUUAUUACCAGUG | CACUGGUAAUAAAGCUUUGGA | [786-806](21/21) |
| 52 | Cross Sp | 21 | AGCAAAAGACGAAAAUGGCUA | UAGCCAUUUUCGUCUUUUGCU | [152-172](21/21) |
| 53 | Cross Sp | 21 | AAUGAGGAACCACCUCCUCCU | AGGAGGAGGUGGUUCCUCAUU | [91-111](21/21) |
| 54 | Cross Sp | 21 | AAUGUACACACUGGUACUUAG | CUAAGUACCAGUGUGUACAUU | [971-991](21/21) |
| 55 | Cross Sp | 21 | UGUGUACAAUGUACACACUGG | CCAGUGUGUACAUUGUACACA | [964-984](21/21) |
| 56 | Cross Sp | 21 | AAAAGCAAAAGACGAAAAUGG | CCAUUUUCGUCUUUUGCUUUU | [149-169](21/21) |
| 57 | Cross Sp | 21 | UCAUGGUGCCAGCCUGACUGA | UCAGUCAGGCUGGCACCAUGA | [47-67](21/21) |
| 58 | Cross Sp | 21 | CGAAUGAGGAACCACCUCCUC | GAGGAGGUGGUUCCUCAUUCG | [89-109](21/21) |
| 59 | Cross Sp | 21 | CCAGCCUGACUGAGAAGAGGA | UCCUCUUCUCAGUCAGGCUGG | [55-75](21/21) |
| 60 | Cross Sp | 21 | GUGCCAGCCUGACUGAGAAGA | UCUUCUCAGUCAGGCUGGCAC | [52-72](21/21) |
| 61 | Cross Sp | 21 | ACUGCCUGGUUGCAGAAGUGC | GCACUUCUGCAACCAGGCAGU | [323-343](21/21) |
| 62 | Cross Sp | 21 | CACUGCCUGGUUGCAGAAGUG | CACUUCUGCAACCAGGCAGUG | [322-342](21/21) |
| 63 | Cross Sp | 21 | CCACUGCCUGGUUGCAGAAGU | ACUUCUGCAACCAGGCAGUGG | [321-341](21/21) |
| 64 | Cross Sp | 21 | GUUCAAGUACAGGGGCCUGGU | ACCAGGCCCCUGUACUUGAAC | [115-135](21/21) |
| 65 | Cross Sp | 21 | CCUGACUGAGAAGAGGACGCU | AGCGUCCUCUUCUCAGUCAGG | [59-79](21/21) |
| 66 | Cross Sp | 21 | UGCCAGCCUGACUGAGAAGAG | CUCUUCUCAGUCAGGCUGGCA | [53-73](21/21) |
| 67 | Cross Sp | 21 | UGGUGCCAGCCUGACUGAGAA | UUCUCAGUCAGGCUGGCACCA | [50-70](21/21) |
| 68 | Cross Sp | 21 | ACCACUGCCUGGUUGCAGAAG | CUUCUGCAACCAGGCAGUGGU | [320-340](21/21) |
| 69 | Cross Sp | 21 | CUGUUCAAGUACAGGGGCCUG | CAGGCCCCUGUACUUGAACAG | [113-133](21/21) |
| 70 | Cross Sp | 21 | GAAUGAGGAACCACCUCCUCC | GGAGGAGGUGGUUCCUCAUUC | [90-110](21/21) |
| 71 | Cross Sp | 21 | CUGACUGAGAAGAGGACGCUC | GAGCGUCCUCUUCUCAGUCAG | [60-80](21/21) |
| 72 | Cross Sp | 21 | AGCCUGACUGAGAAGAGGACG | CGUCCUCUUCUCAGUCAGGCU | [57-77](21/21) |
| 73 | Cross Sp | 21 | AUGGUGCCAGCCUGACUGAGA | UCUCAGUCAGGCUGGCACCAU | [49-69](21/21) |
| 74 | Cross Sp | 21 | CAGCCUGACUGAGAAGAGGAC | GUCCUCUUCUCAGUCAGGCUG | [56-76](21/21) |
| 75 | Cross Sp | 21 | UGACUGAGAAGAGGACGCUCC | GGAGCGUCCUCUUCUCAGUCA | [61-81](21/21) |
| 76 | Cross Sp | 21 | UGUUCAAGUACAGGGGCCUGG | CCAGGCCCCUGUACUUGAACA | [114-134](21/21) |
| 77 | Human | 21 | CAAGGUGGUGUGAUCUUAAUA | UAUUAAGAUCACACCACCUUG | [906-926](21/21) |
| 78 | Human | 21 | CCAGUGGCGUUGUUGCAUGUU | AACAUGCAACAACGCCACUGG | [801-821](21/21) |
| 79 | Human | 21 | CAACCUCCAUUCUAUUUUAGA | UCUAAAAUAGAAUGGAGGUUG | [701-721](21/21) |
| 80 | Human | 21 | GUAGAUGACAACCUCCAUUCU | AGAAUGGAGGUUGUCAUCUAC | [693-713](21/21) |
| 81 | Human | 21 | GGAGUGCUGCUGUAGAUGACA | UGUCAUCUACAGCAGCACUCC | [682-702](21/21) |
| 82 | Human | 21 | CCAUCCAUCAACUUCUAUAAA | UUUAUAGAAGUUGAUGGAUGG | [568-588](21/21) |
| 83 | Human | 21 | GAACCAUCCAUCAACUUCUAU | AUAGAAGUUGAUGGAUGGUUC | [565-585](21/21) |
| 84 | Human | 21 | GCAUAGGAUCAGAAAUUCUGA | UCAGAAUUUCUGAUCCUAUGC | [476-496](21/21) |
| 85 | Human | 21 | ACAUGGAAGAACAAGUAAUCU | AGAUUACUUGUUCUUCCAUGU | [251-271](21/21) |
| 86 | Human | 21 | GUACUUAGAGUUUCUGUUUGA | UCAAACAGAAACUCUAAGUAC | [984-1004](21/21) |
| 87 | Human | 21 | GGUGGUGUGAUCUUAAUAUAU | AUAUAUUAAGAUCACACCACC | [909-929](21/21) |
| 88 | Human | 21 | CAGAUCUUUCUCCUUGAAUAU | AUAUUCAAGGAGAAAGAUCUG | [871-891](21/21) |
| 89 | Human | 21 | GUCAGAUCUUUCUCCUUGAAU | AUUCAAGGAGAAAGAUCUGAC | [869-889](21/21) |
| 90 | Human | 21 | CAGUUUGGAGAGUCAGAUCUU | AAGAUCUGACUCUCCAAACUG | [858-878](21/21) |
| 91 | Human | 21 | CAAUCUCAGAUGCAGUUUGGA | UCCAAACUGCAUCUGAGAUUG | [846-866](21/21) |
| 92 | Human | 21 | GGAGCUGGCUAAAUAUGAAUA | UAUUCAUAUUUAGCCAGCUCC | [231-251](21/21) |
| 93 | Human | 21 | CUGUUUAAAGUGGCAAUCUCA | UGAGAUUGCCACUUUAAACAG | [833-853](21/21) |
| 94 | Human | 21 | CAAGGAGCUGGCUAAAUAUGA | UCAUAUUUAGCCAGCUCCUUG | [228-248](21/21) |
| 95 | Human | 21 | GUGGCGUUGUUGCAUGUUUGA | UCAAACAUGCAACAACGCCAC | [804-824](21/21) |
| 96 | Human | 21 | GAGCACCCAUUCCAAAGCUUU | AAAGCUUUGGAAUGGGUGCUC | [776-796](21/21) |
| 97 | Human | 21 | CUGAUCAAGGAGCUGGCUAAA | UUUAGCCAGCUCCUUGAUCAG | [223-243](21/21) |
| 98 | Human | 21 | CUAUGCUGUUUGUAGUGAAAU | AUUUCACUACAAACAGCAUAG | [747-767](21/21) |
| 99 | Human | 21 | CAACUUCUCUUGCUUUCUAUG | CAUAGAAAGCAAGAGAAGUUG | [731-751](21/21) |
| 100 | Human | 21 | CCAACUUCUCUUGCUUUCUAU | AUAGAAAGCAAGAGAAGUUGG | [730-750](21/21) |
| 101 | Human | 21 | GAUGACAACCUCCAUUCUAUU | AAUAGAAUGGAGGUUGUCAUC | [696-716](21/21) |
| 102 | Human | 21 | CAAGGAGUACUUGCUAAAAAU | AUUUUUAGCAAGUACUCCUUG | [645-665](21/21) |
| 103 | Human | 21 | GACAAGGAGUACUUGCUAAAA | UUUUAGCAAGUACUCCUUGUC | [643-663](21/21) |
| 104 | Human | 21 | CGACAAGGAGUACUUGCUAAA | UUUAGCAAGUACUCCUUGUCG | [642-662](21/21) |
| 105 | Human | 21 | AGAGGGUUGGAGACUGUUCAA | UUGAACAGUCUCCAACCCUCU | [618-638](21/21) |
| 106 | Human | 21 | UGAAGAGGGUUGGAGACUGUU | AACAGUCUCCAACCCUCUUCA | [615-635](21/21) |
| 107 | Human | 21 | GCUUCUGAUCUGUCCAGUGAA | UUCACUGGACAGAUCAGAAGC | [598-618](21/21) |
| 108 | Human | 21 | GAAGAGGUGCUUCUGAUCUGU | ACAGAUCAGAAGCACCUCUUC | [590-610](21/21) |
| 109 | Human | 21 | GAAUGAACCAUCCAUCAACUU | AAGUUGAUGGAUGGUUCAUUC | [561-581](21/21) |
| 110 | Human | 21 | GGAAUGAACCAUCCAUCAACU | AGUUGAUGGAUGGUUCAUUCC | [560-580](21/21) |
| 111 | Human | 21 | GGAUCAGAAAUUCUGAAGAAU | AUUCUUCAGAAUUUCUGAUCC | [481-501](21/21) |
| 112 | Human | 21 | GCUUUGGCAUAGGAUCAGAAA | UUUCUGAUCCUAUGCCAAAGC | [470-490](21/21) |
| 113 | Human | 21 | AUAGAGGCUUUGGCAUAGGAU | AUCCUAUGCCAAAGCCUCUAU | [464-484](21/21) |
| 114 | Human | 21 | GACUUCUUCGUGAUGAGUGAU | AUCACUCAUCACGAAGAAGUC | [442-462](21/21) |
| 115 | Human | 21 | GAGGACUUCUUCGUGAUGAGU | ACUCAUCACGAAGAAGUCCUC | [439-459](21/21) |
| 116 | Human | 21 | GGUUUUGCCAUGUACUAUUUU | AAAAUAGUACAUGGCAAAACC | [382-402](21/21) |
| 117 | Human | 21 | GGACACAGCAUUGUUGGUUUU | AAAACCAACAAUGCUGUGUCC | [367-387](21/21) |
| 118 | Human | 21 | GGAAGGACACACAGCAUUGUUGG | CCAACAAUGCUGUGUCCUUCC | [363-383](21/21) |
| 119 | Human | 21 | GAAAAAGAUCUGCUAGAAGAU | AUCUUCUAGCAGAUCUUUUUC | [277-297](21/21) |
| 120 | Human | 21 | GAAUACAUGGAAGAACAAGUA | UACUUGUUCUUCCAUGUAUUC | [247-267](21/21) |
| 121 | Human | 21 | CGUGAGUCAUUUAAAUGUGUA | UACACAUUUAAAUGACUCACG | [949-969](21/21) |
| 122 | Human | 21 | CAUUCUCGUGAGUCAUUUAAA | UUUAAAUGACUCACGAGAAUG | [943-936](21/21) |
| 123 | Human | 21 | AACUUCAUUCUCGUGAGUCAU | AUGACUCACGAGAAUGAAGUU | [938-958](21/21) |
| 124 | Human | 21 | CAACAAGGUGGUGUGAUCUUA | UAAGAUCACACCACCUUGUUG | [903-923](21/21) |
| 125 | Human | 21 | AACAACAAGGUGGUGUGAUCU | AGAUCACACCACCUUGUUGUU | [901-921](21/21) |
| 126 | Human | 21 | CUCCUUGAAUAUCUUUCGAUA | UAUCGAAAGAUAUUCAAGGAG | [880-900](21/21) |
| 127 | Human | 21 | GAGAGUCAGAUCUUUCUCCUU | AAGGAGAAAGAUCUGACUCUC | [865-885](21/21) |
| 128 | Human | 21 | GGAGAGUCAGAUCUUUCUCCU | AGGAGAAAGAUCUGACUCUCC | [864-884](21/21) |
| 129 | Human | 21 | GCAGUUUGGAGAGUCAGAUCU | AGAUCUGACUCUCCAAACUGC | [857-877](21/21) |
| 130 | Human | 21 | AGAUGCAGUUUGGAGAGUCAG | CUGACUCUCCAAACUGCAUCU | [853-873](21/21) |
| 131 | Human | 21 | GCAAUCUCAGAUGCAGUUUGG | CCAAACUGCAUCUGAGAUUGC | [845-865](21/21) |
| 132 | Human | 21 | UGGCAAUCUCAGAUGCAGUUU | AAACUGCAUCUGAGAUUGCCA | [843-863](21/21) |
| 133 | Human | 21 | AAAGUGGCAAUCUCAGAUGCA | UGCAUCUGAGAUUGCCACUUU | [839-859](21/21) |
| 134 | Human | 21 | GUCUGUUUAAAGUGGCAAUCU | AGAUUGCCACUUUAAACAGAC | [831-851](21/21) |
| 135 | Human | 21 | GUUUGAAAUGAGGUCUGUUUA | UAAACAGACCUCAUUUCAAAC | [819-839](21/21) |
| 136 | Human | 21 | GUUGCAUGUUUGAAAUGAGGU | ACCUCAUUUCAAACAUGCAAC | [812-832](21/21) |
| 137 | Human | 21 | CGUUGUUGCAUGUUUGAAAUG | CAUUUCAAACAUGCAACAACG | [808-828](21/21) |
| 138 | Human | 21 | GCGUUGUUGCAUGUUUGAAAU | AUUUCAAACAUGCAACAACGC | [807-827](21/21) |
| 139 | Human | 21 | UUACCAGUGGCGUUGUUGCAU | AUGCAACAACGCCACUGGUAA | [798-818](21/21) |
| 140 | Human | 21 | AUCAAGGAGCUGGCUAAAUAU | AUAUUUAGCCAGCUCCUUGAU | [226-246](21/21) |
| 141 | Human | 21 | CCCAUUCCAAAGCUUUAUUAC | GUAAUAAAGCUUUGGAAUGGG | [781-801](21/21) |
| 142 | Human | 21 | GCACCCAUUCCAAAGCUUUAU | AUAAAGCUUUGGAAUGGGUGC | [778-798](21/21) |
| 143 | Human | 21 | UGAGCACCCAUUCCAAAGCUU | AAGCUUUGGAAUGGGUGCUCA | [775-795](21/21) |
| 144 | Human | 21 | AGAAUGAGCACCCAUUCCAAA | UUUGGAAUGGGUGCUCAUUCU | [771-791](21/21) |
| 145 | Human | 21 | UAGAAUGAGCACCCAUUCCAA | UUGGAAUGGGUGCUCAUUCUA | [770-790](21/21) |
| 146 | Human | 21 | UUGCUUUCUAUGCUGUUUGUA | UACAAACAGCAUAGAAAGCAA | [740-760](21/21) |
| 147 | Human | 21 | CUUGCUUUCUAUGCUGUUUGU | ACAAACAGCAUAGAAAGCAAG | [739-759](21/21) |
| 148 | Human | 21 | UCCCAACUUCUCUUGCUUUCU | AGAAAGCAAGAGAAGUUGGGA | [728-748](21/21) |
| 149 | Human | 21 | AUUCCCAACUUCUCUUGCUUU | AAAGCAAGAGAAGUUGGGAAU | [726-746](21/21) |
| 150 | Human | 21 | GAAUAAAUUCCCAACUUCUCU | AGAGAAGUUGGGAAUUUAUUC | [720-740](21/21) |
| 151 | Human | 21 | GACAACCUCCAUUCUAUUUUA | UAAAAUAGAAUGGAGGUUGUC | [699-719](21/21) |
| 152 | Human | 21 | GCUGUAGAUGACAACCUCCAU | AUGGAGGUUGUCAUCUACAGC | [690-710](21/21) |
| 153 | Human | 21 | GUGCUGCUGUAGAUGACAACC | GGUUGUCAUCUACAGCAGCAC | [685-705](21/21) |
| 154 | Human | 21 | AUACUGCGGCUGAUCAAGGAG | CUCCUUGAUCAGCCGCAGUAU | [214-234](21/21) |
| 155 | Human | 21 | AUCGACAAGGAGUACUUGCUA | UAGCAAGUACUCCUUGUCGAU | [640-660](21/21) |
| 156 | Human | 21 | CAAGAUCGACAAGGAGUACUU | AAGUACUCCUUGUCGAUCUUG | [636-656](21/21) |
| 157 | Human | 21 | CUGUUCAAGAUCGACAAGGAG | CUCCUUGUCGAUCUUGAACAG | [631-651](21/21) |
| 158 | Human | 21 | AGACUGUUCAAGAUCGACAAG | CUUGUCGAUCUUGAACAGUCU | [628-648](21/21) |
| 159 | Human | 21 | GGUUGGAGACUGUUCAAGAUC | GAUCUUGAACAGUCUCCAACC | [622-642](21/21) |
| 160 | Human | 21 | UGACAUACUGCGGCUGAUCAA | UUGAUCAGCCGCAGUAUGUCA | [210-230](21/21) |
| 161 | Human | 21 | UUCUGAUCUGUCCAGUGAAGA | UCUUCACUGGACAGAUCAGAA | [600-620](21/21) |
| 162 | Human | 21 | GAGGUGCUUCUGAUCUGUCCA | UGGACAGAUCAGAAGCACCUC | [593-613](21/21) |
| 163 | Human | 21 | AAAGAAGAGGUGCUUCUGAUC | GAUCAGAAGCACCUCUUCUUU | [587-607](21/21) |
| 164 | Human | 21 | CAACUUCUAUAAAAGAAGAGG | CCUCUUCUUUUAUAGAAGUUG | [576-596](21/21) |
| 165 | Human | 21 | UGAACCAUCCAUCAACUUCUA | UAGAAGUUGAUGGAUGGUUCA | [564-584](21/21) |
| 166 | Human | 21 | CAGAAUGGAAUGAACCAUCCA | UGGAUGGUUCAUUCCAUUCUG | [554-574](21/21) |
| 167 | Human | 21 | GUAGCAGAAUGGAAUGAACCA | UGGUUCAUUCCAUUCUGCUAC | [550-570](21/21) |
| 168 | Human | 21 | GGUAGCAGAAUGGAAUGAACC | GGUUCAUUCCAUUCUGCUACC | [549-569](21/21) |
| 169 | Human | 21 | ACUUCUUGGUAGCAGAAUGGA | UCCAUUCUGCUACCAAGAAGU | [542-562](21/21) |
| 170 | Human | 21 | AGCAGCAUGCACUUCUUGGUA | UACCAAGAAGUGCAUGCUGCU | [532-552](21/21) |

| | | | | |
|---|---|---|---|---|
| | | Oligo | | |
| No | Source | Length | mouse 42476316 | rat 56090258 |

FOR 19
STRETCHES:

| | | | | |
|---|---|---|---|---|
| 1 | Cross Sp | 19 | [860-878](19/19) | [914-932](19/19) |
| 2 | Cross Sp | 19 | [214-232](19/19) | [267-285](19/19) |
| 3 | Cross Sp | 19 | [887-905](19/19) | [941-959](19/19) |
| 4 | Cross Sp | 19 | [465-483](19/19) | [518-536](19/19) |
| 5 | Cross Sp | 19 | [234-252](19/19) | [287-305](19/19) |
| 6 | Cross Sp | 19 | [1057-1075](19/19) | [1109-1127](19/19) |
| 7 | Cross Sp | 19 | [1051-1069](19/19) | [1103-1121](19/19) |
| 8 | Cross Sp | 19 | [1048-1066](19/19) | [1100-1118](19/19) |
| 9 | Cross Sp | 19 | [1041-1059](19/19) | [1093-1111](19/19) |
| 10 | Cross Sp | 19 | [1024-1042](19/19) | [1076-1094](19/19) |
| 11 | Cross Sp | 19 | [886-904](19/19) | [940-958](19/19) |
| 12 | Cross Sp | 19 | [865-883](19/19) | [919-937](19/19) |
| 13 | Cross Sp | 19 | [571-589](19/19) | [624-642](19/19) |
| 14 | Cross Sp | 19 | [469-487](19/19) | [522-540](19/19) |
| 15 | Cross Sp | 19 | [464-482](19/19) | [517-535](19/19) |
| 16 | Cross Sp | 19 | [313-331](19/19) | [366-384](19/19) |
| 17 | Cross Sp | 19 | [224-242](19/19) | [277-295](19/19) |
| 18 | Cross Sp | 19 | [220-238](19/19) | [273-291](19/19) |
| 19 | Cross Sp | 19 | [219-237](19/19) | [272-290](19/19) |
| 20 | Cross Sp | 19 | [216-234](19/19) | [269-287](19/19) |
| 21 | Cross Sp | 19 | [213-231](19/19) | [266-284](19/19) |
| 22 | Cross Sp | 19 | [166-184](19/19) | [219-237](19/19) |
| 23 | Cross Sp | 19 | [1058-1076](19/19) | [1110-1128](19/19) |
| 24 | Cross Sp | 19 | [1050-1068](19/19) | [1102-1120](19/19) |
| 25 | Cross Sp | 19 | [1043-1061](19/19) | [1095-1113](19/19) |
| 26 | Cross Sp | 19 | [863-881](19/19) | [917-935](19/19) |
| 27 | Cross Sp | 19 | [680-698](19/19) | [733-751](19/19) |
| 28 | Cross Sp | 19 | [615-633](19/19) | [668-686](19/19) |
| 29 | Cross Sp | 19 | [572-590](19/19) | [625-643](19/19) |
| 30 | Cross Sp | 19 | [470-488](19/19) | [523-541](19/19) |
| 31 | Cross Sp | 19 | [399-417](19/19) | [452-470](19/19) |
| 32 | Cross Sp | 19 | [311-329](19/19) | [364-382](19/19) |
| 33 | Cross Sp | 19 | [232-250](19/19) | [285-303](19/19) |
| 34 | Cross Sp | 19 | [230-248](19/19) | [283-301](19/19) |
| 35 | Cross Sp | 19 | [229-247](19/19) | [282-300](19/19) |
| 36 | Cross Sp | 19 | [227-245](19/19) | [280-298](19/19) |
| 37 | Cross Sp | 19 | [223-241](19/19) | [276-294](19/19) |
| 38 | Cross Sp | 19 | [221-239](19/19) | [274-292](19/19) |
| 39 | Cross Sp | 19 | [218-236](19/19) | [271-289](19/19) |
| 40 | Cross Sp | 19 | [215-233](19/19) | [268-286](19/19) |
| 41 | Cross Sp | 19 | [137-155](19/19) | [190-208](19/19) |
| 42 | Cross Sp | 19 | [1055-1073](19/19) | [1107-1125](19/19) |
| 43 | Cross Sp | 19 | [1054-1072](19/19) | [1106-1124](19/19) |
| 44 | Cross Sp | 19 | [1052-1070](19/19) | [1104-1122](19/19) |
| 45 | Cross Sp | 19 | [1047-1065](19/19) | [1099-1117](19/19) |
| 46 | Cross Sp | 19 | [1045-1063](19/19) | [1097-1115](19/19) |
| 47 | Cross Sp | 19 | [1038-1056](19/19) | [1090-1108](19/19) |
| 48 | Cross Sp | 19 | [1026-1044](19/19) | [1078-1096](19/19) |
| 49 | Cross Sp | 19 | [866-884](19/19) | [920-938](19/19) |
| 50 | Cross Sp | 19 | [864-882](19/19) | [918-936](19/19) |
| 51 | Cross Sp | 19 | [861-879](19/19) | [915-933](19/19) |
| 52 | Cross Sp | 19 | [573-591](19/19) | [626-644](19/19) |
| 53 | Cross Sp | 19 | [570-588](19/19) | [623-641](19/19) |
| 54 | Cross Sp | 19 | [314-332](19/19) | [367-385](19/19) |
| 55 | Cross Sp | 19 | [233-251](19/19) | [286-304](19/19) |
| 56 | Cross Sp | 19 | [231-249](19/19) | [284-302](19/19) |
| 57 | Cross Sp | 19 | [228-246](19/19) | [281-299](19/19) |
| 58 | Cross Sp | 19 | [222-240](19/19) | [275-293](19/19) |
| 59 | Cross Sp | 19 | [190-208](19/19) | [243-261](19/19) |
| 60 | Cross Sp | 19 | [1056-1074](19/19) | [1108-1126](19/19) |
| 61 | Cross Sp | 19 | [1053-1071](19/19) | [1105-1123](19/19) |
| 62 | Cross Sp | 19 | [1044-1062](19/19) | [1096-1114](19/19) |
| 63 | Cross Sp | 19 | [1039-1057](19/19) | [1091-1109](19/19) |
| 64 | Cross Sp | 19 | [1037-1055](19/19) | [1089-1107](19/19) |
| 65 | Cross Sp | 19 | [1036-1054](19/19) | [1088-1106](19/19) |
| 66 | Cross Sp | 19 | [862-880](19/19) | [916-934](19/19) |
| 67 | Cross Sp | 19 | [614-632](19/19) | [667-685](19/19) |
| 68 | Cross Sp | 19 | [471-489](19/19) | [524-542](19/19) |
| 69 | Cross Sp | 19 | [226-244](19/19) | [279-297](19/19) |
| 70 | Cross Sp | 19 | [217-235](19/19) | [270-288](19/19) |
| 71 | Cross Sp | 19 | [167-185](19/19) | [220-238](19/19) |
| 72 | Cross Sp | 19 | [133-151](19/19) | [186-204](19/19) |
| 73 | Cross Sp | 19 | [1049-1067](19/19) | [1101-1119](19/19) |
| 74 | Cross Sp | 19 | [1046-1064](19/19) | [1098-1116](19/19) |
| 75 | Cross Sp | 19 | [1042-1060](19/19) | [1094-1112](19/19) |
| 76 | Cross Sp | 19 | [1040-1058](19/19) | [1092-1110](19/19) |
| 77 | Cross Sp | 19 | [1035-1053](19/19) | [1087-1105](19/19) |
| 78 | Cross Sp | 19 | [681-699](19/19) | [734-752](19/19) |
| 79 | Cross Sp | 19 | [164-182](19/19) | [217-235](19/19) |
| 80 | Cross Sp | 19 | [128-146](19/19) | [181-199](19/19) |
| 81 | Cross Sp | 19 | [400-418](19/19) | [453-471](19/19) |
| 82 | Cross Sp | 19 | [397-415](19/19) | [450-468](19/19) |
| 83 | Cross Sp | 19 | [169-187](19/19) | [222-240](19/19) |
| 84 | Cross Sp | 19 | [130-148](19/19) | [183-201](19/19) |
| 85 | Cross Sp | 19 | [396-414](19/19) | [449-467](19/19) |
| 86 | Cross Sp | 19 | [165-183](19/19) | [218-236](19/19) |
| 87 | Cross Sp | 19 | [134-152](19/19) | [187-205](19/19) |
| 88 | Cross Sp | 19 | [132-150](19/19) | [185-203](19/19) |
| 89 | Cross Sp | 19 | [131-149](19/19) | [184-202](19/19) |
| 90 | Cross Sp | 19 | [125-143](19/19) | [178-196](19/19) |
| 91 | Cross Sp | 19 | [193-211](19/19) | [246-264](19/19) |
| 92 | Cross Sp | 19 | [189-207](19/19) | [242-260](19/19) |
| 93 | Cross Sp | 19 | [123-141](19/19) | [176-194](19/19) |
| 94 | Cross Sp | 19 | [139-157](19/19) | [192-210](19/19) |
| 95 | Cross Sp | 19 | [136-154](19/19) | [189-207](19/19) |
| 96 | Cross Sp | 19 | [129-147](19/19) | [182-200](19/19) |
| 97 | Cross Sp | 19 | [401-419](19/19) | [454-472](19/19) |
| 98 | Cross Sp | 19 | [398-416](19/19) | [451-469](19/19) |
| 99 | Cross Sp | 19 | [192-210](19/19) | [245-263](19/19) |
| 100 | Cross Sp | 19 | [395-413](19/19) | [448-466](19/19) |
| 101 | Human | 19 | — | — |
| 102 | Human | 19 | [606-624](18/19) | [659-677](18/19) |
| 103 | Human | 19 | — | — |
| 104 | Human | 19 | [492-504](13/13) | [545-563](18/19) |
| 105 | Human | 19 | [325-342](17/18) | [378-396](18/19) |
| 106 | Human | 19 | — | — |
| 107 | Human | 19 | [975-985](11/11) | [1029-1039](11/11) |
| 108 | Human | 19 | — | — |
| 109 | Human | 19 | [353-363](11/11) | [406-416](11/11) |
| 110 | Human | 19 | [353-363](11/11) | [406-416](11/11) |
| 111 | Human | 19 | — | — |
| 112 | Human | 19 | [311-325](15/15) | [364-378](15/15) |
| 113 | Human | 19 | [304-322](18/19) | [357-375](18/19) |
| 114 | Human | 19 | [303-321](18/19) | [356-374](18/19) |
| 115 | Human | 19 | [856-874](19/19) | [914-928](15/15) |
| 116 | Human | 19 | [854-867](14/14) | — |
| 117 | Human | 19 | [812-827](15/16) | [863-881](18/19) |
| 118 | Human | 19 | [780-790](11/11) | — |
| 119 | Human | 19 | [776-790](15/15) | — |
| 120 | Human | 19 | [720-738](18/19) | [781-791](11/11) |
| 121 | Human | 19 | — | — |
| 122 | Human | 19 | — | — |
| 123 | Human | 19 | — | — |
| 124 | Human | 19 | — | — |
| 125 | Human | 19 | — | — |
| 126 | Human | 19 | [650-666](16/17) | [703-719](16/17) |
| 127 | Human | 19 | [645-660](15/16) | [698-713](15/16) |
| 128 | Human | 19 | [644-660](16/17) | [697-713](16/17) |
| 129 | Human | 19 | — | — |
| 130 | Human | 19 | [626-644](19/19) | — |
| 131 | Human | 19 | [622-640](19/19) | [675-693](18/19) |
| 132 | Human | 19 | [609-627](18/19) | [662-680](18/19) |
| 133 | Human | 19 | [278-291](14/14) | [331-344](14/14) |
| 134 | Human | 19 | — | — |
| 135 | Human | 19 | [557-575](18/19) | [610-628](18/19) |
| 136 | Human | 19 | [526-540](15/15) | [580-593](14/14) |
| 137 | Human | 19 | [491-504](14/14) | [544-558](15/15) |
| 138 | Human | 19 | [449-459](11/11) | [498-516](19/19) |
| 139 | Human | 19 | — | [493-511](19/19) |
| 140 | Human | 19 | — | [419-435](16/17) |
| 141 | Human | 19 | — | — |
| 142 | Human | 19 | — | — |
| 143 | Human | 19 | — | — |
| 144 | Human | 19 | [322-336](15/15) | [375-389](15/15) |
| 145 | Human | 19 | [321-336](16/16) | [374-389](16/16) |
| 146 | Human | 19 | [1024-1038](15/15) | [1076-1090](15/15) |
| 147 | Human | 19 | [1018-1036](18/19) | [1070-1088](18/19) |
| 148 | Human | 19 | [1016-1034](18/19) | [1068-1086](18/19) |
| 149 | Human | 19 | [1011-1029](18/19) | [1063-1081](18/19) |
| 150 | Human | 19 | — | — |
| 151 | Human | 19 | — | — |
| 152 | Human | 19 | — | — |
| 153 | Human | 19 | — | — |
| 154 | Human | 19 | [975-992](17/18) | [1029-1039](11/11) |
| 155 | Human | 19 | [975-991](16/17) | [1029-1039](11/11) |
| 156 | Human | 19 | — | — |
| 157 | Human | 19 | — | — |
| 158 | Human | 19 | — | — |
| 159 | Human | 19 | [353-363](11/11) | [406-416](11/11) |
| 160 | Human | 19 | — | — |
| 161 | Human | 19 | — | — |
| 162 | Human | 19 | — | — |
| 163 | Human | 19 | — | — |
| 164 | Human | 19 | — | — |
| 165 | Human | 19 | [311-326](16/16) | [364-379](16/16) |
| 166 | Human | 19 | [908-921](14/14) | [962-973](12/12) |
| 167 | Human | 19 | [908-919](12/12) | [962-973](12/12) |
| 168 | Human | 19 | — | — |
| 169 | Human | 19 | [886-902](17/17) | [938-956](19/19) |
| 170 | Human | 19 | [886-900](15/15) | [936-954](19/19) |
| 171 | Human | 19 | [880-898](18/19) | [934-952](19/19) |
| 172 | Human | 19 | [859-877](19/19) | [914-931](18/18) |
| 173 | Human | 19 | [855-873](19/19) | [909-927](18/19) |
| 174 | Human | 19 | [854-872](19/19) | [908-926](18/19) |
| 175 | Human | 19 | [854-868](15/15) | — |
| 176 | Human | 19 | — | — |
| 177 | Human | 19 | [835-847](13/13) | [886-901](16/16) |
| 178 | Human | 19 | — | [886-898](13/13) |
| 179 | Human | 19 | — | [886-897](12/12) |
| 180 | Human | 19 | — | — |
| 181 | Human | 19 | — | — |
| 182 | Human | 19 | [817-828](12/12) | [871-882](12/12) |
| 183 | Human | 19 | [812-828](16/17) | [864-882](18/19) |
| 184 | Human | 19 | — | — |
| 185 | Human | 19 | — | — |
| 186 | Human | 19 | — | — |

FOR 21
STRETCHES:

| | | | | |
|---|---|---|---|---|
| 1 | Cross Sp | 21 | [221-241](21/21) | [274-294](21/21) |
| 2 | Cross Sp | 21 | [213-233](21/21) | [266-286](21/21) |
| 3 | Cross Sp | 21 | [1050-1070](21/21) | [1102-1122](21/21) |
| 4 | Cross Sp | 21 | [1048-1068](21/21) | [1100-1120](21/21) |
| 5 | Cross Sp | 21 | [1046-1066](21/21) | [1098-1118](21/21) |
| 6 | Cross Sp | 21 | [1044-1064](21/21) | [1096-1116](21/21) |
| 7 | Cross Sp | 21 | [860-880](21/21) | [914-934](21/21) |
| 8 | Cross Sp | 21 | [571-591](21/21) | [624-644](21/21) |
| 9 | Cross Sp | 21 | [469-489](21/21) | [522-542](21/21) |
| 10 | Cross Sp | 21 | [465-485](21/21) | [518-538](21/21) |
| 11 | Cross Sp | 21 | [464-484](21/21) | [517-537](21/21) |
| 12 | Cross Sp | 21 | [230-250](21/21) | [283-303](21/21) |
| 13 | Cross Sp | 21 | [229-249](21/21) | [282-302](21/21) |
| 14 | Cross Sp | 21 | [226-246](21/21) | [279-299](21/21) |
| 15 | Cross Sp | 21 | [224-244](21/21) | [277-297](21/21) |
| 16 | Cross Sp | 21 | [214-234](21/21) | [267-287](21/21) |
| 17 | Cross Sp | 21 | [1056-1076](21/21) | [1108-1128](21/21) |
| 18 | Cross Sp | 21 | [1043-1063](21/21) | [1095-1115](21/21) |
| 19 | Cross Sp | 21 | [1042-1062](21/21) | [1094-1114](21/21) |
| 20 | Cross Sp | 21 | [1041-1061](21/21) | [1093-1113](21/21) |
| 21 | Cross Sp | 21 | [1040-1060](21/21) | [1092-1112](21/21) |
| 22 | Cross Sp | 21 | [1039-1059](21/21) | [1091-1111](21/21) |
| 23 | Cross Sp | 21 | [1037-1057](21/21) | [1089-1109](21/21) |
| 24 | Cross Sp | 21 | [1024-1044](21/21) | [1076-1096](21/21) |
| 25 | Cross Sp | 21 | [570-590](21/21) | [623-643](21/21) |
| 26 | Cross Sp | 21 | [570-590](21/21) | [623-643](21/21) |
| 27 | Cross Sp | 21 | [312-332](21/21) | [365-385](21/21) |
| 28 | Cross Sp | 21 | [311-331](21/21) | [364-384](21/21) |
| 29 | Cross Sp | 21 | [220-240](21/21) | [273-293](21/21) |
| 30 | Cross Sp | 21 | [219-239](21/21) | [272-292](21/21) |
| 31 | Cross Sp | 21 | [218-238](21/21) | [271-291](21/21) |
| 32 | Cross Sp | 21 | [216-236](21/21) | [269-289](21/21) |
| 33 | Cross Sp | 21 | [215-235](21/21) | [268-288](21/21) |
| 34 | Cross Sp | 21 | [1055-1075](21/21) | [1107-1271](21/21) |
| 35 | Cross Sp | 21 | [1054-1074](21/21) | [1106-1126](21/21) |
| 36 | Cross SP | 21 | [1053-1073](21/21) | [1105-1125](21/21) |
| 37 | Cross Sp | 21 | [1052-1072](21/21) | [1104-1124](21/21) |
| 38 | Cross Sp | 21 | [1049-1069](21/21) | [1101-1121](21/21) |
| 39 | Cross Sp | 21 | [1047-1067](21/21) | [1099-1119](21/21) |
| 40 | Cross Sp | 21 | [1036-1056](21/21) | [1088-1108](21/21) |
| 41 | Cross Sp | 21 | [863-883](21/21) | [917-937](21/21) |
| 42 | Cross Sp | 21 | [862-882](21/21) | [916-936](21/21) |
| 43 | Cross Sp | 21 | [861-881](21/21) | [915-935](21/21) |
| 44 | Cross Sp | 21 | [468-488](21/21) | [521-541](21/21) |
| 45 | Cross Sp | 21 | [313-333](21/21) | [366-386](21/21) |
| 46 | Cross Sp | 21 | [227-247](21/21) | [280-300](21/21) |
| 47 | Cross Sp | 21 | [217-237](21/21) | [270-290](21/21) |
| 48 | Cross Sp | 21 | [164-184](21/21) | [217-237](21/21) |
| 49 | Cross Sp | 21 | [1051-1071](21/21) | [1103-1123](21/21) |
| 50 | Cross Sp | 21 | [1035-1055](21/21) | [1087-1107](21/21) |
| 51 | Cross Sp | 21 | [864-884](21/21) | [918-938](21/21) |
| 52 | Cross Sp | 21 | [228-248](21/21) | [281-301](21/21) |
| 53 | Cross Sp | 21 | [167-187](21/21) | [220-240](21/21) |
| 54 | Cross Sp | 21 | [1045-1065](21/21) | [1097-1117](21/21) |
| 55 | Cross Sp | 21 | [1038-1058](21/21) | [1090-1110](21/21) |
| 56 | Cross Sp | 21 | [225-245](21/21) | [278-298](21/21) |
| 57 | Cross Sp | 21 | [123-143](21/21) | [176-196](21/21) |
| 58 | Cross Sp | 21 | [165-185](21/21) | [218-238](21/21) |
| 59 | Cross Sp | 21 | [131-151](21/21) | [184-204](21/21) |
| 60 | Cross Sp | 21 | [128-148](21/21) | [181-201](21/21) |
| 61 | Cross Sp | 21 | [399-419](21/21) | [452-472](21/21) |
| 62 | Cross Sp | 21 | [398-418](21/21) | [451-471](21/21) |
| 63 | Cross Sp | 21 | [397-417](21/21) | [450-470](21/21) |
| 64 | Cross Sp | 21 | [191-211](21/21) | [244-264](21/21) |
| 65 | Cross Sp | 21 | [135-155](21/21) | [188-208](21/21) |
| 66 | Cross Sp | 21 | [129-149](21/21) | [182-202](21/21) |
| 67 | Cross Sp | 21 | [126-146](21/21) | [179-199](21/21) |
| 68 | Cross Sp | 21 | [396-416](21/21) | [449-469](21/21) |
| 69 | Cross Sp | 21 | [189-209](21/21) | [242-262](21/21) |
| 70 | Cross Sp | 21 | [166-186](21/21) | [219-239](21/21) |
| 71 | Cross Sp | 21 | [136-156](21/21) | [189-209](21/21) |
| 72 | Cross Sp | 21 | [133-153](21/21) | [186-206](21/21) |
| 73 | Cross Sp | 21 | [125-145](21/21) | [178-198](21/21) |
| 74 | Cross Sp | 21 | [132-152](21/21) | [185-205](21/21) |
| 75 | Cross Sp | 21 | [137-157](21/21) | [190-210](21/21) |
| 76 | Cross Sp | 21 | [190-210](21/21) | [243-263](21/21) |
| 77 | Human | 21 | — | — |
| 78 | Human | 21 | [879-899](20/21) | [933-953](21/21) |
| 79 | Human | 21 | [780-790](11/11) | — |
| 80 | Human | 21 | [776-790](15/15) | — |
| 81 | Human | 21 | — | — |
| 82 | Human | 21 | [644-660](16/17) | [697-713](16/17) |
| 83 | Human | 21 | [641-660](19/20) | [694-713](19/20) |
| 84 | Human | 21 | [554-568](15/15) | [607-621](15/15) |
| 85 | Human | 21 | — | — |
| 86 | Human | 21 | [1058-1076](19/19) | [1110-1128](19/19) |
| 87 | Human | 21 | — | — |
| 88 | Human | 21 | [353-363](11/11) | [406-416](11/11) |
| 89 | Human | 21 | [353-363](11/11) | [406-416](11/11) |
| 90 | Human | 21 | — | — |
| 91 | Human | 21 | — | — |
| 92 | Human | 21 | [311-327](17/17) | [364-380](17/17) |
| 93 | Human | 21 | [911-925](15/15) | — |
| 94 | Human | 21 | [304-324](20/21) | [357-377](20/21) |
| 95 | Human | 21 | [886-902](17/17) | [936-956](21/21) |
| 96 | Human | 21 | [854-874](21/21) | [908-928](20/21) |
| 97 | Human | 21 | [299-319](20/21) | [352-372](20/21) |
| 98 | Human | 21 | [835-845](11/11) | [886-899](14/14) |
| 99 | Human | 21 | [812-828](16/17) | [863-882](19/20) |
| 100 | Human | 21 | [812-828](16/17) | [863-882](19/20) |
| 101 | Human | 21 | [776-790](15/15) | — |
| 102 | Human | 21 | [725-741](17/17) | [781-794](14/14) |
| 103 | Human | 21 | [719-739](20/21) | [772-792](19/21) |
| 104 | Human | 21 | [719-738](19/20) | [772-791](18/20) |
| 105 | Human | 21 | — | — |
| 106 | Human | 21 | — | — |
| 107 | Human | 21 | [674-694](20/21) | [727-747](20/21) |
| 108 | Human | 21 | [666-686](20/21) | [719-739](20/21) |
| 109 | Human | 21 | [637-657](20/21) | [690-710](20/21) |
| 110 | Human | 21 | [636-656](20/21) | [689-709](20/21) |
| 111 | Human | 21 | [557-577](20/21) | [610-630](20/21) |
| 112 | Human | 21 | [546-566](20/21) | [599-619](20/21) |
| 113 | Human | 21 | [542-560](18/19) | — |
| 114 | Human | 21 | [518-538](21/21) | [571-591](20/21) |
| 115 | Human | 21 | [518-535](18/18) | — |
| 116 | Human | 21 | [464-478](15/15) | [511-531](21/21) |
| 117 | Human | 21 | [443-459](16/17) | [496-516](21/21) |
| 118 | Human | 21 | [440-459](19/20) | [493-512](20/20) |
| 119 | Human | 21 | — | — |
| 120 | Human | 21 | [323-342](19/20) | [376-396](20/21) |
| 121 | Human | 21 | [1024-1043](20/20) | [1076-1095](20/20) |
| 122 | Human | 21 | [1017-1037](20/21) | [1069-1089](20/21) |
| 123 | Human | 21 | [1012-1032](20/21) | [1064-1084](20/21) |
| 124 | Human | 21 | [975-994](19/20) | [1029-1039](11/11) |
| 125 | Human | 21 | [975-992](17/18) | [1029-1039](11/11) |
| 126 | Human | 21 | — | — |
| 127 | Human | 21 | [353-363](11/11) | [406-416](11/11) |
| 128 | Human | 21 | [353-363](11/11) | [406-416](11/11) |
| 129 | Human | 21 | — | — |
| 130 | Human | 21 | — | — |
| 131 | Human | 21 | — | — |
| 132 | Human | 21 | — | — |
| 133 | Human | 21 | — | — |
| 134 | Human | 21 | [909-925](17/17) | [963-979](16/17) |
| 135 | Human | 21 | — | — |
| 136 | Human | 21 | [890-905](16/16) | [944-959](16/16) |
| 137 | Human | 21 | [886-905](20/20) | [940-959](20/20) |
| 138 | Human | 21 | [886-905](20/20) | [939-959](21/21) |
| 139 | Human | 21 | [876-896](20/21) | [930-950](21/21) |
| 140 | Human | 21 | [302-322](20/21) | [355-375](20/21) |
| 141 | Human | 21 | [859-879](21/21) | [914-933](20/20) |
| 142 | Human | 21 | [856-876](21/21) | [914-930](17/17) |
| 143 | Human | 21 | [854-873](20/20) | [907-927](20/21) |
| 144 | Human | 21 | [854-869](16/16) | — |
| 145 | Human | 21 | [854-868](15/15) | — |
| 146 | Human | 21 | [818-828](11/11) | [872-882](11/11) |
| 147 | Human | 21 | [817-828](12/12) | [871-882](12/12) |
| 148 | Human | 21 | — | — |
| 149 | Human | 21 | — | — |
| 150 | Human | 21 | — | — |
| 151 | Human | 21 | [778-790](13/13) | — |
| 152 | Human | 21 | [776-789](14/14) | — |
| 153 | Human | 21 | — | — |
| 154 | Human | 21 | [293-309](16/17) | [346-362](16/17) |
| 155 | Human | 21 | [719-736](17/18) | — |
| 156 | Human | 21 | — | — |
| 157 | Human | 21 | — | — |
| 158 | Human | 21 | — | — |
| 159 | Human | 21 | — | — |
| 160 | Human | 21 | — | — |
| 161 | Human | 21 | [680-696](17/17) | [733-749](17/17) |
| 162 | Human | 21 | — | — |
| 163 | Human | 21 | [663-683](20/21) | [716-736](20/21) |
| 164 | Human | 21 | [652-672](20/21) | [705-725](20/21) |
| 165 | Human | 21 | [640-660](20/21) | [639-713](20/21) |
| 166 | Human | 21 | [630-648](19/19) | [683-701](18/19) |
| 167 | Human | 21 | [626-646](21/21) | [679-699](20/21) |
| 168 | Human | 21 | [625-645](21/21) | [678-698](20/21) |
| 169 | Human | 21 | [618-638](21/21) | [671-691](20/21) |
| 170 | Human | 21 | [608-628](20/21) | [661-681](20/21) |

Table L below shows further siRNA compounds specific for the SSG1 gene that have been selected according the present invention.

TABLE L

| | | | | | |
|---|---|---|---|---|---|
| No. | Source | length | Sense siRNA | AntiSense siRNA | human 41152073 |

FOR 19
STRETCHES:

| | | | | | |
|---|---|---|---|---|---|
| 1 | Cross Sp | 19 | CCAUGGAUACCCUUACUGA | UCAGUAAGGGUAUCCAUGG | [3650-3668](19/19) |
| 2 | Cross Sp | 19 | GGAUGACUACCGUCAUCAU | AUGAUGACGGUAGUCAUCC | [3620-3638](19/19) |
| 3 | Cross Sp | 19 | CUGCCCAGAAGAUGAGUAU | AUACUCAUCUUCUGGGCAG | [3551-3569](19/19) |
| 4 | Cross Sp | 19 | CCCGAAUCAAAGAUAUGGA | UCCAUAUCUUUGAUUCGGG | [3049-3067](19/19) |
| 5 | Cross Sp | 19 | CACCAUGGAUACCCUUACU | AGUAAGGGUAUCCAUGGUG | [3648-3666](19/19) |
| 6 | Cross Sp | 19 | GCUGCCCAGAAGAUGAGUA | UACUCAUCUUCUGGGCAGC | [3550-3568](19/19) |
| 7 | Cross Sp | 19 | ACUUCUCCAUGCUUCUAGU | ACUAGAAGCAUGGAGAAGU | [3403-3421](19/19) |
| 8 | Cross Sp | 19 | GAGUACUUCUCCAUGCUUC | GAAGCAUGGAGAAGUACUC | [3399-3417](19/19) |
| 9 | Cross Sp | 19 | GAACUGUUCCCAAUUAAUG | CAUUAAUUGGGAACAGUUC | [3303-3321](19/19) |
| 10 | Cross Sp | 19 | AGAACUGUUCCCAAUUAAU | AUUAAUUGGGAACAGUUCU | [3302-3320](19/19) |
| 11 | Cross Sp | 19 | UAGAACUGUUCCCAAUUAA | UUAAUUGGGAACAGUUCUA | [3301-3319](19/19) |
| 12 | Cross Sp | 19 | UUAGAACUGUUCCCAAUUA | UAAUUGGGAACAGUUCUAA | [3300-3318](19/19) |
| 13 | Cross Sp | 19 | GAAGACUGGGCCUAUUCAC | GUGAAUAGGCCCAGUCUUC | [3183-3201](19/19) |
| 14 | Cross Sp | 19 | GCAGAAGAAGGAGGGCAUU | AAUGCCCUCCUUCUUCUGC | [3071-3089](19/19) |
| 15 | Cross Sp | 19 | ACCAUGGAUACCCUUACUG | CAGUAAGGGUAUCCAUGGU | [3649-3667](19/19) |
| 16 | Cross Sp | 19 | AUGACUUCUUCAUGGUGCU | AGCACCAUGAAGAAGUCAU | [2944-2962](19/19) |
| 17 | Cross Sp | 19 | GAUGACUACCGUCAUCAUG | CAUGAUGACGGUAGUCAUC | [3621-3639](19/19) |
| 18 | Cross Sp | 19 | AGGAUGACUACCGUCAUCA | UGAUGACGGUAGUCAUCCU | [3619-3637](19/19) |
| 19 | Cross Sp | 19 | CACCAAGGAUACCAGGAUG | CAUCCUGGUAUCCUUGGUG | [3594-3612](19/19) |
| 20 | Cross Sp | 19 | GCCCAGAAGAUGAGUAUGC | GCAUACUCAUCUUCUGGGC | [3553-3571](19/19) |
| 21 | Cross Sp | 19 | GAAGGCAAACGAAGACUCC | GGAGUCUUCGUUUGCCUUC | [2673-2691](19/19) |
| 22 | Cross Sp | 19 | GUACUUCUCCAUGCUUCUA | UAGAAGCAUGGAGAAGUAC | [3401-3419](19/19) |
| 23 | Cross Sp | 19 | CUGUUCCCAAUUAAUGGGA | UCCCAUUAAUUGGGAACAG | [3306-3324](19/19) |
| 24 | Cross Sp | 19 | AAGACUGGGCCUAUUCACA | UGUGAAUAGGCCCAGUCUU | [3184-3202](19/19) |
| 25 | Cross Sp | 19 | GAAAGAGGACCCAAGGAGA | UCUCCUUGGGUCCUCUUUC | [1748-1766](19/19) |
| 26 | Cross Sp | 19 | AUGACUACCGUCAUCAUGA | UCAUGAUGACGGUAGUCAU | [3622-3640](19/19) |
| 27 | Cross Sp | 19 | ACCAAGGAUACCAGGAUGG | CCAUCCUGGUAUCCUUGGU | [3595-3613](19/19) |
| 28 | Cross Sp | 19 | UACUUCUCCAUGCUUCUAG | CUAGAAGCAUGGAGAAGUA | [3402-3420](19/19) |
| 29 | Cross Sp | 19 | AGUACUUCUCCAUGCUUCU | AGAAGCAUGGAGAAGUACU | [3400-3418](19/19) |
| 30 | Cross Sp | 19 | GUUCCCAAUUAAUGGGAGC | GCUCCCAUUAAUUGGGAAC | [3308-3326](19/19) |
| 31 | Cross Sp | 19 | ACUGUUCCCAAUUAAUGGG | CCCAUUAAUUGGGAACAGU | [3305-3323](19/19) |
| 32 | Cross Sp | 19 | GUUGCUGGUGAUCUCUGCU | AGCAGAGAUCACCAGCAAC | [3155-3173](19/19) |
| 33 | Cross Sp | 19 | UCCCGAAUCAAAGAUAUGG | CCAUAUCUUUGAUUCGGGA | [3048-3066](19/19) |
| 34 | Cross Sp | 19 | UUCCCAAUUAAUGGGAGCU | AGCUCCCAUUAAUUGGGAA | [3309-3327](19/19) |
| 35 | Cross Sp | 19 | UGUUCCCAAUUAAUGGGAG | CUCCCAUUAAUUGGGAACA | [3307-3325](19/19) |
| 36 | Cross Sp | 19 | AACUGUUCCCAAUUAAUGG | CCAUUAAUUGGGAACAGUU | [3304-3322](19/19) |
| 37 | Cross Sp | 19 | UUGCUGGUGAUCUCUGCUC | GAGCAGAGAUCACCAGCAA | [3156-3174](19/19) |
| 38 | Cross Sp | 19 | UGCCCAGAAGAUGAGUAUG | CAUACUCAUCUUCUGGGCA | [3552-3570](19/19) |
| 39 | Cross Sp | 19 | AAGGCAAACGAAGACUCCU | AGGAGUCUUCGUUUGCCUU | [2674-2692](19/19) |
| 40 | Cross Sp | 19 | UCCCAAUUAAUGGGAGCUC | GAGCUCCCAUUAAUUGGGA | [3310-3328](19/19) |
| 41 | Cross Sp | 19 | GAUGCGCUGCCCAGAAGAU | AUCUUCUGGGCAGCGCAUC | [3545-3563](19/19) |
| 42 | Cross Sp | 19 | GGUUGCUGGUGAUCUCUGC | GCAGAGAUCACCAGCAACC | [3154-3172](19/19) |
| 43 | Cross Sp | 19 | CGCUGCCCAGAAGAUGAGU | ACUCAUCUUCUGGGCAGCG | [3549-3567](19/19) |
| 44 | Cross Sp | 19 | UGCGCUGCCCAGAAGAUGA | UCAUCUUCUGGGCAGCGCA | [3547-3565](19/19) |
| 45 | Cross Sp | 19 | UGCUGGUGAUCUCUGCUCC | GGAGCAGAGAUCACCAGCA | [3157-3175](19/19) |
| 46 | Cross Sp | 19 | AUGCGCUGCCCAGAAGAUG | CAUCUUCUGGGCAGCGCAU | [3546-3564](19/19) |
| 47 | Human | 19 | GCAGCGAGAAGAAGAAAGA | UCUUUCUUCUUCUCGCUGC | [1735-1753](19/19) |
| 48 | Human | 19 | GAAGGGCUUUGUCCAGAAA | UUUCUGGACAAAGCCCUUC | [1637-1655](19/19) |
| 49 | Human | 19 | GCAUGGUGCUGCUGAAGAA | UUCUUCAGCAGCACCAUGC | [1516-1534](19/19) |
| 50 | Human | 19 | CUAGCCUCAUCCCUAAGCU | AGCUUAGGGAUGAGGCUAG | [1462-1480](19/19) |
| 51 | Human | 19 | CAACAUCCUUGCCAGCUUU | AAAGCUGGCAAGGAUGUUG | [1232-1250](19/19) |
| 52 | Human | 19 | GUACAGCACAGGCAUACCA | UGGUAUGCCUGUGCUGUAC | [4435-4453](19/19) |
| 53 | Human | 19 | CUAGUAGGCUGCUAUGGAA | UUCCAUAGCAGCCUACUAG | [4374-4392](19/19) |
| 54 | Human | 19 | CCACGGGAGUCCAGUAAUA | UAUUACUGGACUCCCGUGG | [4194-4212](19/19) |
| 55 | Human | 19 | GCAGAAAUAUGUAACCUUA | UAAGGUUACAUAUUUCUGC | [3669-3687](19/19) |
| 56 | Human | 19 | CAACUUCGGAGACAGGAAA | UUUCCUGUCUCCGAAGUUG | [3504-3522](19/19) |
| 57 | Human | 19 | GCAAAGAGGACAAAAAGCA | UGCUUUUUGUCCUCUUUGC | [3094-3112](19/19) |
| 58 | Human | 19 | GGAGGGCAUUGUUUGCAAA | UUUGCAAACAAUGCCCUCC | [3080-3098](19/19) |
| 59 | Human | 19 | GUACCAAUAACAAUGAAGU | ACUUCAUUGUUAUUGGUAC | [3000-3018](19/19) |
| 60 | Human | 19 | GGAUCUGAGAGUCAAGCAA | UUGCUUGACUCUCAGAUCC | [2972-2990](19/19) |
| 61 | Human | 19 | GGAUGAUGAAGACUUGGUA | UACCAAGUCUUCAUCAUCC | [2876-2894](19/19) |
| 62 | Human | 19 | CCAGGAAAAUCUCUGUGAU | AUCACAGAGAUUUUCCUGG | [2776-2794](19/19) |
| 63 | Human | 19 | GCAACAACGUGAUGAAUAU | AUAUUCAUCACGUUGUUGC | [2732-2750](19/19) |
| 64 | Human | 19 | CCACUCUGGAGGAACCAAA | UUUGGUUCCUCCAGAGUGG | [1006-1024](19/19) |
| 65 | Human | 19 | CCAAGGCUGAGAACAAUAU | AUAUUGUUCUCAGCCUUGG | [2707-2725](19/19) |
| 66 | Human | 19 | GAAGAAAAAAGGAGGUAAA | UUUACCUCCUUUUUUCUUC | [2567-2585](19/19) |
| 67 | Human | 19 | GCAAGCAAGAGAAAGAGAA | UUCUCUUUCUCUUGCUUGC | [2542-2560](19/19) |
| 68 | Human | 19 | GGGUGAAGGUCCUGAGAAA | UUUCUCAGGACCUUCACCC | [1795-1813](19/19) |
| 69 | Human | 19 | GCUUUGUCCAGAAAUGUAA | UUACAUUUCUGGACAAAGC | [1642-1660](19/19) |
| 70 | Human | 19 | GGAUCGAGAAGAUCAGGCA | UGCCUGAUCUUCUCGAUCC | [1618-1636](19/19) |
| 71 | Human | 19 | GUAGGAUCGAGAAGAUCAG | CUGAUCUUCUCGAUCCUAC | [1615-1633](19/19) |
| 72 | Human | 19 | CCAUGUACGAGGUCAUCGA | UCGAUGACCUCGUACAUGG | [1582-1600](19/19) |
| 73 | Human | 19 | AGAGGCACAUCCAACAGAU | AUCUGUUGGAUGUGCCUCU | [1357-1375](19/19) |
| 74 | Human | 19 | GAAGGACGAUGUGUACUGU | ACAGUACACAUCGUCCUUC | [1328-1346](19/19) |
| 75 | Human | 19 | GCUAGUGUGUGGAUCAGAA | UUCUGAUCCACACACUAGC | [863-881](19/19) |
| 76 | Human | 19 | GCAGGGAAGAACAGAGUAU | AUACUCUGUUCUUCCCUGC | [1251-1269](19/19) |
| 77 | Human | 19 | UCAGCUCGGUCAAGAAUGU | ACAUUCUUGACCGAGCUGA | [1185-1203](19/19) |
| 78 | Human | 19 | GUGAGUCACACAAAAAAAA | UUUUUUUUGUGUGACUCAC | [4511-4529](19/19) |
| 79 | Human | 19 | GGAUAUCGCAAUAAAGUGA | UCACUUUAUUGCGAUAUCC | [4496-4514](19/19) |
| 80 | Human | 19 | CAGUUCCAGACCACCAUAA | UUAUGGUGGUCUGGAACUG | [4471-4489](19/19) |
| 81 | Human | 19 | AGAUAUUACAGGAUCAGUU | AACUGAUCCUGUAAUAUCU | [4457-4475](19/19) |
| 82 | Human | 19 | AUACCAUGGAGAUAUUACA | UGUAAUAUCUCCAUGGUAU | [4448-4446](19/19) |
| 83 | Human | 19 | GGCAUACCAUGGAGAUAUU | AAUAUCUCCAUGGUAUGCC | [4445-4463](19/19) |
| 84 | Human | 19 | CUUGACCCCUCACACUUUA | UAAAGUGUGAGGGGUCAAG | [4410-4428](19/19) |
| 85 | Human | 19 | GGAAUUUCUGGCAUGAAAA | UUUUCAUGCCAGAAAUUCC | [4389-4407](19/19) |
| 86 | Human | 19 | GAAAUCCUCACCAGAAUCU | AGAUUCUGGUGAGGAUUUC | [4322-4340](19/19) |
| 87 | Human | 19 | CCCUGCUUCUGUUAUUUUU | AAAAAUAACAGAAGCAGGG | [4272-4290](19/19) |
| 88 | Human | 19 | GCCCAGCUUUGUUAUCAUA | UAUGAUAACAAAGCUGGGC | [4242-4260](19/19) |
| 89 | Human | 19 | CCAAGCCCAGCUUUGUUAU | AUAACAAAGCUGGGCUUGG | [4238-4256](19/19) |
| 90 | Human | 19 | GGCCACAAAAGCCAGAGAA | UUCUCUGGCUUUUGUGGCC | [4215-4233](19/19) |
| 91 | Human | 19 | CAGUAAUAUUGGCCACAAA | UUUGUGGCCAAUAUUACUG | [4205-4223](19/19) |
| 92 | Human | 19 | AGAUAUCUCUGCUUUGUAU | AUACAAAGCAGAGAUAUCU | [4151-4169](19/19) |
| 93 | Human | 19 | UGAAAUGCCAGUCUGAAAA | UUUUCAGACUGGCAUUUCA | [4126-4144](19/19) |
| 94 | Human | 19 | CAAGAAACAUCUGAAAAGA | UCUUUUCAGAUGUUUCUUG | [4058-4076](19/19) |
| 95 | Human | 19 | CCACAAUGUUUUCAAGAAA | UUUCUUGAAAACAUUGUGG | [4046-4064](19/19) |
| 96 | Human | 19 | GAUCACACCACAAUGUUUU | AAAACAUUGUGGUGUGAUC | [4039-4057](19/19) |
| 97 | Human | 19 | AAAGAUCACACCACAAUGU | ACAUUGUGGUGUGAUCUUU | [4036-4054](19/19) |
| 98 | Human | 19 | GACCUUAUUUGGCAUAUAA | UUAUAUGCCAAAUAAGGUC | [3948-3966](19/19) |
| 99 | Human | 19 | GGGCUCUAGUGCUAAAUAA | UUAUUUAGCACUAGAGCCC | [3887-3905](19/19) |
| 100 | Human | 19 | CAAACUUUCACCUAUUCAU | AUGAAUAGGUGAAAGUUUG | [3807-3825](19/19) |
| 101 | Human | 19 | CAGUGUUUUUCUAAGACUA | UAGUCUUAGAAAAACACUG | [3779-3797](19/19) |
| 102 | Human | 19 | GCAGCUGCUAAAACUACAU | AUGUAGUUUUAGCAGCUGC | [3707-3725](19/19) |
| 103 | Human | 19 | CUCUGCAGCUGCUAAAACU | AGUUUUAGCAGCUGCAGAG | [3703-3721](19/19) |
| 104 | Human | 19 | AGAGUUAUCACCAUGGAUA | UAUCCAUGGUGAUAACUCU | [3640-3658](19/19) |
| 105 | Human | 19 | CUACCGUCAUCAUGAGAGU | ACUCUCAUGAUGACGGUAG | [3626-3644](19/19) |
| 106 | Human | 19 | AUAGUUACCACCAAGGAUA | UAUCCUUGGUGGUAACUAU | [3586-3604](19/19) |
| 107 | Human | 19 | GCUAUGGUUACCAUAGUUA | UAACUAUGGUAACCAUAGC | [3574-3592](19/19) |
| 108 | Human | 19 | GCAGGCUAUGGUUACCAUA | UAUGGUAACCAUAGCCUGC | [3570-3588](19/19) |
| 109 | Human | 19 | UGAGUAUGCAGGCUAUGGU | ACCAUAGCCUGCAUACUCA | [3563-3581](19/19) |
| 110 | Human | 19 | GACAGGAAAUGGCGAUUCA | UGAAUCGCCAUUUCCUGUC | [3514-3532](19/19) |
| 111 | Human | 19 | CGAUUUAAUUGAUUCGAUG | CAUCGAAUCAAUUAAAUCG | [3485-3503](19/19) |
| 112 | Human | 19 | GGUCCAUGGUGAUUGUGUA | UACACAAUCACCAUGGACC | [3466-3484](19/19) |
| 113 | Human | 19 | CCAAUGUGGUCCAUGGUGA | UCACCAUGGACCACAUUGG | [3459-3477](19/19) |
| 114 | Human | 19 | GUCAAAUCCUGGUAUCCUU | AAGGAUACCAGGAUUUGAC | [3438-3456](19/19) |
| 115 | Human | 19 | GGAAAAGACGGAAAUGUCA | UGACAUUUCCGUCUUUUCC | [3423-3441](19/19) |
| 116 | Human | 19 | CAUGCUUCUAGUCGGAAAA | UUUUCCGACUAGAAGCAUG | [3410-3428](19/19) |
| 117 | Human | 19 | GAAAGACAUUCGUAACUAU | AUAGUUACGAAUGUCUUUC | [3365-3383](19/19) |
| 118 | Human | 19 | CCCAUUUGGUGAAAGACAU | AUGUCUUUCACCAAAUGGG | [3355-3373](19/19) |
| 119 | Human | 19 | CAGCCCAUUUGGUGAAAGA | UCUUUCACCAAAUGGGCUG | [3352-3370](19/19) |
| 120 | Human | 19 | GGAAGUUGGGGGAGUGUUA | UAACACUCCCCCAACUUCC | [3284-3302](19/19) |
| 121 | Human | 19 | GAAGCUUUUAGGCGUUGGA | UCCAACGCCUAAAAGCUUC | [3263-3281](19/19) |
| 122 | Human | 19 | CCAUUCUGAAGCUUUUAGG | CCUAAAAGCUUCAGAAUGG | [3256-3274](19/19) |
| 123 | Human | 19 | UAACCAUUCUGAAGCUUUU | AAAAGCUUCAGAAUGGUUA | [3253-3271](19/19) |
| 124 | Human | 19 | CGCCACAUAACCAUUCUGA | UCAGAAUGGUUAUGUGGCG | [3246-3264](19/19) |
| 125 | Human | 19 | GUCUGCGCCACAUAACCAU | AUGGUUAUGUGGCGCAGAC | [3241-3259](19/19) |
| 126 | Human | 19 | CUGCUCCUAACGAUGAAGA | UCUUCAUCGUUAGGAGCAG | [3169-3187](19/19) |
| 127 | Human | 19 | GAUCUCUGCUCCUAACGAU | AUCGUUAGGAGCAGAGAUC | [3164-3182](19/19) |
| 128 | Human | 19 | GAAGAAGGAGGGCAUUGUU | AACAAUGCCCUCCUUCUUC | [3074-3092](19/19) |
| 129 | Human | 19 | CCAGUCCCGAAUCAAAGAU | AUCUUUGAUUCGGGACUGG | [3044-3062](19/19) |
| 130 | Human | 19 | UGAAGUCUGUGUUUGAUCU | AGAUCAAACACAGACUUCA | [3013-3031](19/19) |
| 131 | Human | 19 | UAACAAUGAAGUCUGUGUU | AACACAGACUUCAUUGUUA | [3007-3025](19/19) |
| 132 | Human | 19 | CUAUGAGGUACCAAUAACA | UGUUAUUGGUACCUCAUAG | [2993-3011](19/19) |
| 133 | Human | 19 | UGAGAGUCAAGCAAUACUA | UAGUAUUGCUUGACUCUCA | [2977-2995](19/19) |
| 134 | Human | 19 | CCUACAAUGACUUCUUCAU | AUGAAGAAGUCAUUGUAGG | [2938-2956](19/19) |
| 135 | Human | 19 | GAAUGACCUACAAUGACUU | AAGUCAUUGUAGGUCAUUC | [2932-2950](19/19) |
| 136 | Human | 19 | GGAAUGACCUACAAUGACU | AGUCAUUGUAGGUCAUUCC | [2931-2949](19/19) |
| 137 | Human | 19 | GUAGACCAGCGUCUCAUCA | UGAUGAGACGCUGGUCUAC | [2892-2910](19/19) |
| 138 | Human | 19 | CCACUUUCAGCUAGAUAAU | AUUAUCUAGCUGAAAGUGG | [2837-2855](19/19) |
| 139 | Human | 19 | CGACCACUUUCAGCUAGAU | AUCUAGCUGAAAGUGGUCG | [2834-2852](19/19) |
| 140 | Human | 19 | UGAAAAUCGACCACUUUCA | UGAAAGUGGUCGAUUUUCA | [2827-2845](19/19) |
| 141 | Human | 19 | CCAUGAAAAUCGACCACUU | AAGUGGUCGAUUUUCAUGG | [2824-2842](19/19) |
| 142 | Human | 19 | ACAGCACCAUGAAAAUCGA | UCGAUUUUCAUGGUGCUGU | [2818-2836](19/19) |
| 143 | Human | 19 | CAAGAUGGCUACCAGGAAA | UUUCCUGGUAGCCAUCUUG | [2765-2783](19/19) |

FOR 21
STRETCHES:

| | | | | | |
|---|---|---|---|---|---|
| 1 | Cross Sp | 21 | CACCAUGGAUACCCUUACU | UCAGUAAGGGUAUCCAUGGUG | [3648-3668](21/21) |
| | | | GA | | |
| 2 | Cross Sp | 21 | GGAUGACUACCGUCAUCAU | UCAUGAUGACGGUAGUCAUCC | [3620-3640](21/21) |
| | | | GA | | |
| 3 | Cross Sp | 21 | GUACUUCUCCAUGCUUCUA | ACUAGAAGCAUGGAGAAGUAC | [3401-3421](21/21) |
| | | | GU | | |
| 4 | Cross Sp | 21 | GCUGCCCAGAAGAUGAGUA | CAUACUCAUCUUCUGGGCAGC | [3550-3570](21/21) |
| | | | UG | | |
| 5 | Cross Sp | 21 | CGCUGCCCAGAAGAUGAGU | AUACUCAUCUUCUGGGCAGCG | [3549-3569](21/21) |
| | | | AU | | |
| 6 | Cross Sp | 21 | GAGUACUUCUCCAUGCUUC | UAGAAGCAUGGAGAAGUACUC | [3399-3419](21/21) |
| | | | UA | | |
| 7 | Cross Sp | 21 | GAACUGUUCCCAAUUAAUG | CCCAUUAAUUGGGAACAGUUC | [3303-3323](21/21) |
| | | | GG | | |
| 8 | Cross Sp | 21 | AGAACUGUUCCCAAUUAAU | CCAUUAAUUGGGAACAGUUCU | [3302-3322](21/21) |
| | | | GG | | |
| 9 | Cross Sp | 21 | UGUUCCCAAUUAAUGGGAG | AGCUCCCAUUAAUUGGGAACA | [3307-3327](21/21) |
| | | | CU | | |
| 10 | Cross Sp | 21 | CUGUUCCCAAUUAAUGGGA | GCUCCCAUUAAUUGGGAACAG | [3306-3326](21/21) |
| | | | GC | | |
| 11 | Cross Sp | 21 | AACUGUUCCCAAUUAAUGG | UCCCAUUAAUUGGGAACAGUU | [3304-3324](21/21) |
| | | | GA | | |
| 12 | Cross Sp | 21 | UAGAACUGUUCCCAAUUAA | CAUUAAUUGGGAACAGUUCUA | [3301-3321](21/21) |
| | | | UG | | |
| 13 | Cross Sp | 21 | AGGAUGACUACCGUCAUCA | CAUGAUGACGGUAGUCAUCCU | [3619-3639](21/21) |
| | | | UG | | |
| 14 | Cross Sp | 21 | ACUGUUCCCAAUUAAUGGG | CUCCCAUUAAUUGUGAACAGU | [3305-3325](21/21) |
| | | | AG | | |
| 15 | Cross Sp | 21 | CUGCCCAGAAGAUGAGUAU | GCAUACUCAUCUUCUGGGCAG | [3551-3571](21/21) |
| | | | GC | | |
| 16 | Cross Sp | 21 | GUUCCCAAUUAAUGGGAGC | GAGCUCCCAUUAAUUGGGAAC | [3308-3328](21/21) |
| | | | UC | | |
| 17 | Cross Sp | 21 | AGUACUUCUCCAUGCUUCU | CUAGAAGCAUGGAGAAGUACU | [3400-3420](21/21) |
| | | | AG | | |
| 18 | Cross Sp | 21 | UGCGCUGCCCAGAAGAUGA | ACUCAUCUUCUGGGCAGCGCA | [3547-3567](21/21) |
| | | | GU | | |
| 19 | Cross Sp | 21 | GAUGCGCUGCCCAGAAGAU | UCAUCUUCUGGGCAGCGCAUC | [3545-3565](21/21) |
| | | | GA | | |
| 20 | Cross Sp | 21 | GCGCUGCCCAGAAGAUGAG | UACUCAUCUUCUGGGCAGCGC | [3548-3568](21/21) |
| | | | UA | | |
| 21 | Cross Sp | 21 | GUUGCUGGUGAUCUCUGCU | GGAGCAGAGAUCACCAGCAAC | [3155-3175](21/21) |
| | | | CC | | |
| 22 | Cross Sp | 21 | AUGCGCUGCCCAGAAGAUG | CUCAUCUUCUGGGCAGCGCAU | [3546-3566](21/21) |
| | | | AG | | |
| 23 | Cross Sp | 21 | GGUUGCUGGUGAUCUCUGC | GAGCAGAGAUCACCAGCAACC | [3154-3174](21/21) |
| | | | UC | | |
| 24 | Human | 21 | GGACGGAAAGUGCCUUUGG | AACCAAAGGCACUUUCCGUCC | [918-938](21/21) |
| | | | UU | | |
| 25 | Human | 21 | CCAUUGGAUUACACAAUGA | UGUCAUUGUGUAAUCCAAUGG | [801-821](21/21) |
| | | | CA | | |
| 26 | Human | 21 | CUACUUGUCCAGAAGGUAA | UUUUACCUUCUGGACAAGUAG | [553-573](21/21) |
| | | | AA | | |
| 27 | Human | 21 | CCAGUAAUAUUGGCCACAA | UUUUGUGGCCAAUAUUACUGG | [4204-4224](21/21) |
| | | | AA | | |
| 28 | Human | 21 | GGAAAGAGUGAGAUUCAGA | UUUCUGAAUCUCACUCUUUCC | [398-418](21/21) |
| | | | AA | | |
| 29 | Human | 21 | GGACCUUAUUUGGCAUAUA | UUUAUAUGCCAAAUAAGUUCC | [3947-3967](21/21) |
| | | | AA | | |
| 30 | Human | 21 | CUACAUUUCCUGCCUUUUU | AGAAAAAGGCAGGAAAUGUAG | [3756-3776](21/21) |
| | | | CU | | |
| 31 | Human | 21 | GUACUACAUUUCCUGCCUU | AAAAGGCAGGAAAUGUAGUAC | [3753-3773](21/21) |
| | | | UU | | |
| 32 | Human | 21 | GCAACUUCGGAGACAGGAA | AUUUCCUGUCUCCGAAGUUGC | [3503-3523](21/21) |
| | | | AU | | |
| 33 | Human | 21 | GUACGGAAUGACCUACAAU | UCAUUGUAGGUCAUUCCGUAC | [2927-2947](21/21) |
| | | | GA | | |
| 34 | Human | 21 | CAACAACAGCACCAUGAAA | AUUUUCAUGGUGCUGUUGUUG | [2813-2833](21/21) |
| | | | AU | | |
| 35 | Human | 21 | CAAGAUGGCUACCAGGAAA | AUUUUCCUGGUAGCCAUCUUG | [2765-2785](21/21) |
| | | | AU | | |
| 36 | Human | 21 | GCAAACGAAGACUCCUUCU | UCAGAAGGAGUCUUCGUUUGC | [2677-2697](21/21) |
| | | | GA | | |
| 37 | Human | 21 | CCAGAGAAGGAGAAGAAAA | UUUUUUCUUCUCCUUCUCUGG | [2451-2471](21/21) |
| | | | AA | | |
| 38 | Human | 21 | GGAGGAGAGAAGCACAGUG | UUCACUGUGCUUCUCUCCUCC | [224-244](21/21) |
| | | | AA | | |
| 39 | Human | 21 | GAAGAAAAAGAGGAGGAAA | UUUUUCCUCCUCUUUUUCUUC | [186-206](21/21) |
| | | | AA | | |
| 40 | Human | 21 | UUACACAAUGACAUGGAGA | AUUCUCCAUGUCAUUGUGUAA | [809-829](21/21) |
| | | | AU | | |
| 41 | Human | 21 | GAGCCACCAUUGGAUUACA | UGUGUAAUCCAAUGGUGGCUC | [795-815](21/21) |
| | | | CA | | |
| 42 | Human | 21 | GCAUUCCAUCUCUAUGAGC | UGGCUCAUAGAGAUGGAAUGC | [780-800](21/21) |
| | | | CA | | |
| 43 | Human | 21 | CCCUCUGCAUUCCAUCUCUAU | AUAGAGAUGGAAUGCAGAGGG | [774-794](21/21) |
| 44 | Human | 21 | GAACCAAACUUCUGAUUUC | AAGAAAUCAGAAGUUUGGUUC | [747-767](21/21) |
| | | | UU | | |
| 45 | Human | 21 | GGAACCAAACUUCUGAUUU | AGAAAUCAGAAGUUUGGUUCC | [746-766](21/21) |
| | | | CU | | |
| 46 | Human | 21 | GAAGUUUCCUUGAUUUCCA | ACUGGAAAUCAAGGAAACUUC | [73-93](21/21) |
| | | | GU | | |
| 47 | Human | 21 | GAAAAGUAGUUCAUCUAGG | UUCCUAGAUGAACUACUUUUC | [716-736](21/21) |
| | | | AA | | |
| 48 | Human | 21 | GGAGGAAAAGUAGUUCAUC | UAGAUGAACUACUUUUCCUCC | [712-732](21/21) |
| | | | UA | | |
| 49 | Human | 21 | GCAGCACACCCCUUUUGUA | AAUACAAAAGGGGUGUGCUGC | [661-681](21/21) |
| | | | UU | | |
| 50 | Human | 21 | GAAGGUAAAAAGACACAGC | UGGCUGUGUCUUUUUACCUUC | [564-584](21/21) |
| | | | CA | | |
| 51 | Human | 21 | CUUGUCCAGAAGGUAAAAA | UCUUUUUACCUUCUGGACAAG | [556-576](21/21) |
| | | | GA | | |
| 52 | Human | 21 | GAAGGACAGCUCCUUUCAC | AGGUGAAAGGAGCUGUCCUUC | [528-548](21/21) |
| | | | CU | | |
| 53 | Human | 21 | UUAGAAGGACAGCUCCUUU | UGAAAGGAGCUGUCCUUCUAA | [525-545](21/21) |
| | | | CA | | |
| 54 | Human | 21 | GGAGAUACAAAGACUUAGA | CUUCUAAGUCUUUGUAUCUCC | [511-531](21/21) |
| | | | AG | | |
| 55 | Human | 21 | AGACAAAUGGAGAUACAAA | UCUUUGUAUCUCCAUUUGUCU | [503-523](21/21) |
| | | | GA | | |
| 56 | Human | 21 | GAGAAACUGCAGACAAAUG | UCCAUUUGUCUGCAGUUUCUC | [493-513](21/21) |
| | | | GA | | |
| 57 | Human | 21 | GAGGAGAAACUGCAGACAA | AUUUGUCUGCAGUUUCUCCUC | [490-510](21/21) |
| | | | AU | | |
| 58 | Human | 21 | CAAUAAAGUGAGUCACACA | UUUGUGUGACUCACUUUAUUG | [4504-4524](21/21) |
| | | | AA | | |
| 59 | Human | 21 | GUUCCAGACCACCAUAAUA | UUUAUUAUGGUGGUCUGGAAC | [4473-4493](21/21) |
| | | | AA | | |
| 60 | Human | 21 | UCAGUUCCAGACCACCAUA | AUUAUGGUGGUCUGGAACUGA | [4470-4490](21/21) |
| | | | AU | | |
| 61 | Human | 21 | GAUCAGUUCCAGACCACCA | UAUGGUGGUCUGGAACUGAUC | [4468-4488](21/21) |
| | | | UA | | |
| 62 | Human | 21 | GCACAGGCAUACCAUGGAG | AUCUCCAUGGUAUGCCUGUGC | [4440-4460](21/21) |
| | | | AU | | |
| 63 | Human | 21 | GCAUGAAAAUUCUUGACCC | AGGGGUCAAGAAUUUUCAUGC | [4399-4419](21/21) |
| | | | CU | | |
| 64 | Human | 21 | GGAAUUUCUGGCAUGAAAA | AAUUUUCAUGCCAGAAAUUCC | [4389-4409](21/21) |
| | | | UU | | |
| 65 | Human | 21 | CUAGUAGGCUGCUAUGGAA | AAUUCCAUAGCAGCCUACUAG | [4374-4394](21/21) |
| | | | UU | | |
| 66 | Human | 21 | CCAGAGUUCUAGUAGGCUG | AGCAGCCUACUAGAACUCUGG | [4366-4386](21/21) |
| | | | CU | | |
| 67 | Human | 21 | CGAUACUUCCAGAGUUCUA | ACUAGAACUCUGGAAGUAUCG | [4358-4378](21/21) |
| | | | GU | | |
| 68 | Human | 21 | CUGCAGCCAAAGGCGAUAC | AAGUAUCGCCUUUGGCUGCAG | [4345-4365](21/21) |
| | | | UU | | |
| 69 | Human | 21 | GGAAAUCCUCACCAGAAUC | CAGAUUCUGGUGAGGAUUUCC | [4321-4341](21/21) |
| | | | UG | | |
| 70 | Human | 21 | GCCCUGCUUCUGUUAUUUU | UAAAAAUAACAGAAGCAGGGC | [4271-4291](21/21) |
| | | | UA | | |
| 71 | Human | 21 | CUUCCUGCCCUGCUUCUGU | UAACAGAAGCAGGGCAGGAAG | [4265-4285](21/21) |
| | | | UA | | |
| 72 | Human | 21 | GAAAGUACCAAGCCCAGCU | AAAGCUGGGCUUGGUACUUUC | [4231-4251](21/21) |
| | | | UU | | |
| 73 | Human | 21 | CCAGGACUGGACUUUAAGG | UCCCUUAAAGUCCAGUCCUGG | [422-442](21/21) |
| | | | GA | | |
| 74 | Human | 21 | GGUGUUUUGCUGAUCUGUU | UUAACAGAUCAGCAAAACACC | [43-63](21/21) |
| | | | AA | | |
| 75 | Human | 21 | AUACCACGGGAGUCCAGUA | AUUACUGGACUCCCGUGGUAU | [4191-4211](21/21) |
| | | | AU | | |
| 76 | Human | 21 | CCACAAUGUUUUCAAGAAA | UGUUUCUUGAAAACAUUGUGG | [4046-4066](21/21) |
| | | | CA | | |
| 77 | Human | 21 | CACACCACAAUGUUUUCAA | UCUUGAAAACAUUGUGGUGUG | [4042-4062](21/21) |
| | | | GA | | |
| 78 | Human | 21 | GAAGGACCUUAUUUGGCAU | AUAUGCCAAAUAAGGUCCUUC | [3944-3964](21/21) |
| | | | AU | | |
| 79 | Human | 21 | GGCGGUGUUUUGCUGAUCU | ACAGAUCAGCAAAACACCGCC | [40-60](21/21) |
| | | | GU | | |
| 80 | Human | 21 | GGGGCUCUAGUGCUAAAUA | AUUAUUUAGCACUAGAGCCCC | [3886-3906](21/21) |
| | | | AU | | |
| 81 | Human | 21 | GGAGGGGCUCUAGUGCUAA | AUUUAGCACUAGAGCCCCUCC | [3883-3903](21/21) |
| | | | AU | | |
| 82 | Human | 21 | GGAACUUUAAAGAGGGAAA | UCUUUCCCUCUUUAAAGUUCC | [384-404](21/21) |
| | | | GA | | |
| 83 | Human | 21 | GCAAACUUUCACCUAUUCA | CAUGAAUAGGUGAAAGUUUGC | [3806-3826](21/21) |
| | | | UG | | |
| 84 | Human | 21 | GGAUCCUGGAACUUUAAAG | CUCUUUAAAGUUCCAGGAUCC | [377-397](21/21) |
| | | | AG | | |
| 85 | Human | 21 | GAAGGAUCCUGGAACUUUA | UUUAAAGUUCCAGGAUCCUUC | [374-394](21/21) |
| | | | AA | | |
| 86 | Human | 21 | CCAGCUCCAUUCUUCCACACU | AGUGUGGAAGAAUGGAGCUGG | [3730-3750](21/21) |
| 87 | Human | 21 | ACAUGUGGCCAGCUCCAUU | AGAAUGGAGCUGGCCACAUGU | [3722-3742](21/21) |
| | | | CU | | |
| 88 | Human | 21 | AGAAGGAUCCUGGAACUUU | UUAAAGUUCCAGGAUCCUUCU | [373-393](21/21) |
| | | | AA | | |
| 89 | Human | 21 | GCAGCUGCUAAAACUACAU | ACAUGUAGUUUUAGCAGCUGC | [3707-3727](21/21) |
| | | | GU | | |
| 90 | Human | 21 | CCUCUGCAGCUGCUAAAAC | UAGUUUUAGCAGCUGCAGAGG | [3702-3722](21/21) |
| | | | UA | | |
| 91 | Human | 21 | GGAGAUAAGAAGGAUCCUG | UCCAGGAUCCUUCUUAUCUCC | [366-386](21/21) |
| | | | GA | | |
| 92 | Human | 21 | CCAGGAUGACUACCGUCAU | UGAUGACGGUAGUCAUCCUGG | [3617-3637](21/21) |
| | | | CA | | |
| 93 | Human | 21 | CAAGGAUACCAGGAUGGUU | GUAACCAUCCUGGUAUCCUUG | [3597-3617](21/21) |
| | | | AC | | |
| 94 | Human | 21 | GGAAAGGGAGAUAAGAAGG | AUCCUUCUUAUCUCCCUUUCC | [360-380](21/21) |
| | | | AU | | |
| 95 | Human | 21 | CCAUGGUGAUUGUGUACGA | AAUCGUACACAAUCACCAUGG | [3469-3489](21/21) |
| | | | UU | | |
| 96 | Human | 21 | CCAAUGUGGUCCAUGGUGA | AAUCACCAUGGACCACAUUGG | [3459-3479](21/21) |
| | | | UU | | |
| 97 | Human | 21 | GGAAAUGUCAAAUCCUGGU | AUACCAGGAUUUGACAUUUCC | [3432-3452](21/21) |
| | | | AU | | |
| 98 | Human | 21 | CUAGUCGGAAAAGACGGAA | AUUUCCGUCUUUUCCGACUAG | [3417-3437](21/21) |
| | | | AU | | |
| 99 | Human | 21 | CCAUGCUUCUAGUCGGAAA | CUUUUCCGACUAGAAGCAUGG | [3409-3429](21/21) |
| | | | AG | | |
| 100 | Human | 21 | GGAGUACUUCUCCAUGCUU | AGAAGCAUGGAGAAGUACUCC | [3398-3418](21/21) |
| | | | CU | | |
| 101 | Human | 21 | CCGGAGUACUUCUCCAUGC | AAGCAUGGAGAAGUACUCCGG | [3396-3416](21/21) |
| | | | UU | | |
| 102 | Human | 21 | GCCCAUUUGGUGAAAGACA | AAUGUCUUUCACCAAAUGGGC | [3354-3374](21/21) |
| | | | UU | | |
| 103 | Human | 21 | CCAGCCCAUUUGGUGAAAG | GUCUUUCACCAAAUGGGCUGG | [3351-3371](21/21) |
| | | | AC | | |
| 104 | Human | 21 | AAAGAGACUAAGCCAGACU | AUAGUCUGGCUUAGUCUCUUU | [336-356](21/21) |
| | | | AU | | |
| 105 | Human | 21 | GUACCAGCCCAUUUGGUGA | UUUCACCAAAUGGGCUGGUAC | [3348-3368](21/21) |
| | | | AA | | |
| 106 | Human | 21 | GUGUUAGAACUGUUCCCAA | AAUUGGGAACAGUUCUAACAC | [3297-3317](21/21) |
| | | | UU | | |
| 107 | Human | 21 | GGAAACAAAGAGACUAAGC | UGGCUUAGUCUCUUUGUUUCC | [330-350](21/21) |
| | | | CA | | |
| 108 | Human | 21 | GCGCCACAUAACCAUUCUG | UUCAGAAUGGUUAUGUGGCGC | [3245-3265](21/21) |
| | | | AA | | |
| 109 | Human | 21 | GGUCUGCGCCACAUAACCA | AAUGGUUAUGUGGCGCAGACC | [3240-3260](21/21) |
| | | | UU | | |
| 110 | Human | 21 | CAGUCCCUGGAGAACUUCC | UAGGAAGUUCUCCAGGGACUG | [3111-3131](21/21) |
| | | | UA | | |
| 111 | Human | 21 | CCAGUCCCGAAUCAAAGAU | AUAUCUUUGAUUCGGGACUGG | [3044-3064](21/21) |
| | | | AU | | |
| 112 | Human | 21 | GUGUUUGAUCUGAUCGAUA | AGUAUCGAUCAGAUCAAACAC | [3021-3041](21/21) |
| | | | CU | | |
| 113 | Human | 21 | CCAAUAACAAUGAAGUCUG | CACAGACUUCAUUGUUAUUGG | [3003-3023](21/21) |
| | | | UG | | |
| 114 | Human | 21 | CAAUACUAUGAGGUACCAA | UAUUGGUACCUCAUAGUAUUG | [2988-3008](21/21) |
| | | | UA | | |
| 115 | Human | 21 | CUGAGAGUCAAGCAAUACU | AUAGUAUUGCUUGACUCUCAG | [2976-2996](21/21) |
| | | | AU | | |
| 116 | Human | 21 | GACUUCUUCAUGGUGCUAA | UGUUAGCACCAUGAAGAAGUC | [2946-2966](21/21) |
| | | | CA | | |
| 117 | Human | 21 | UGACCUACAAUGACUUCUU | UGAAGAAGUCAUUGUAGGUCA | [2935-2955](21/21) |
| | | | CA | | |
| 118 | Human | 21 | GGAAUGACCUACAAUGACU | GAAGUCAUUGUAGGUCAUUCC | [2931-2951](21/21) |
| | | | UC | | |
| 119 | Human | 21 | GAGUACGGAAUGACCUACA | AUUGUAGGUCAUUCCGUACUC | [2925-2945](21/21) |
| | | | AU | | |
| 120 | Human | 21 | CAGCGAGCUGAGGAAAGAG | UACUCUUUCCUCAGCUCGCUG | [2909-2929](21/21) |
| | | | UA | | |
| 121 | Human | 21 | CGGGUGAAGGUCCUGAGAA | UUUUCUCAGGACCUUCACCCG | [1794-1814](21/21) |
| | | | AA | | |
| 122 | Human | 21 | CUGGGCAGCGAGAAGAAGA | UUUCUUCUUCUCGCUGCCCAG | [1731-1751](21/21) |
| | | | AA | | |
| 123 | Human | 21 | AGAAGGGCUUUGUCCAGAA | AUUUCUGGACAAAGCCCUUCU | [1636-1656](21/21) |
| | | | AU | | |
| 124 | Human | 21 | GAAGCCAUGUACGAGGUCA | GAUGACCUCGUACAUGGCUUC | [1578-1598](21/21) |
| | | | UC | | |
| 125 | Human | 21 | CAACAGAUUGUGCUCUUCC | GUGGAAGAGCACAAUCUGUUG | [1368-1388](21/21) |
| | | | AC | | |
| 126 | Human | 21 | GAGAGGCACAUCCAACAGA | AAUCUGUUGGAUGUGCCUCUC | [1356-1376](21/21) |
| | | | UU | | |
| 127 | Human | 21 | GGAGAGGCACAUCCAACAG | AUCUGUUGGAUGUGCCUCUCC | [1355-1375](21/21) |
| | | | AU | | |
| 128 | Human | 21 | GGAAGAACAGAGUAUGGGU | UGACCCAUACUCUGUUCUUCC | [1255-1275](21/21) |
| | | | CA | | |
| 129 | Human | 21 | CCAGCUUUGCAGGGAAGAA | UGUUCUUCCCUGCAAAGCUGG | [1243-1263](21/21) |
| | | | CA | | |
| 130 | Human | 21 | CCAACAUCCUUGCCAGCUU | CAAAGCUGGCAAGGAUGUUGG | [1231-1251](21/21) |
| | | | UG | | |
| 131 | Human | 21 | CUCAGCUCGGUCAAGAAUG | AACAUUCUUGACCGAGCUGAG | [1184-1204](21/21) |
| | | | UU | | |
| 132 | Human | 21 | CCGCGUGAGAUGAUCAGAG | AUCUCUGAUCAUCUCACGCGG | [1155-1175](21/21) |
| | | | AU | | |
| 133 | Human | 21 | CUCAUCAGCGAGCUGAGGA | UUUCCUCAGCUCGCUGAUGAG | [2904-2924](21/21) |
| | | | AA | | |
| 134 | Human | 21 | GAGUGGUGGAUGAUGAAGA | AGUCUUCAUCAUCCACCACUC | [2869-2889](21/21) |
| | | | CU | | |
| 135 | Human | 21 | GACCACUUUCAGCUAGAUA | AUUAUCUAGCUGAAAGUGGUC | [2835-2855](21/21) |
| | | | AU | | |
| 136 | Human | 21 | CAACAGCACCAUGAAAAUC | UCGAUUUUCAUGGUGCUGUUG | [2816-2836](21/21) |
| | | | GA | | |
| 137 | Human | 21 | GCAAGAUGGCUACCAGGAA | UUUUCCUGGUAGCCAUCUUGC | [2764-2784](21/21) |
| | | | AA | | |
| 138 | Human | 21 | CAAGGCUGAGAACAAUAUG | UACAUAUUGUUCUCAGCCUUG | [2708-2728](21/21) |
| | | | UA | | |
| 139 | Human | 21 | CGAAGACUCCUUCUGAUCA | AGUGAUCAGAAGGAGUCUUCG | [2682-2702](21/21) |
| | | | CU | | |
| 140 | Human | 21 | GGUCCUUUGAAGGCAAACG | UUCGUUUGCCUUCAAAGGACC | [2665-2685](21/21) |
| | | | AA | | |
| 141 | Human | 21 | UCACGCAGAGUCCCAAGAA | ACUUCUUGGGACUCUGCGUGA | [2626-2646](21/21) |
| | | | GU | | |
| 142 | Human | 21 | CUUCACGCAGAGUCCCAAG | UUCUUGGGACUCUGCGUGAAG | [2624-2644](21/21) |
| | | | AA | | |
| 143 | Human | 21 | UCAGAAACCCACCAACAAA | UGUUUGUUGGUGGGUUUCUGA | [2603-2623](21/21) |
| | | | CA | | |
| 144 | Human | 21 | GAACAGGAUGGCUAUCAGA | UUUCUGAUAGCCAUCCUGUUC | [2589-2609](21/21) |
| | | | AA | | |
| 145 | Human | 21 | CAGAACAGGAUGGCUAUCA | UCUGAUAGCCAUCCUGUUCUG | [2587-2607](21/21) |
| | | | GA | | |
| 146 | Human | 21 | GAGAAGAGCAAGAAGAAAA | UUUUUUCUUCUUGCUCUUCUC | [2556-2576](21/21) |
| | | | AA | | |
| 147 | Human | 21 | GAAAGAGAAGAGCAAGAAG | UUCUUCUUGCUCUUCUCUUUC | [2552-2572](21/21) |
| | | | AA | | |
| 148 | Human | 21 | CAAGAGAAAGAGAAGAGCA | CUUGCUCUUCUCUUUCUCUUG | [2547-2567](21/21) |
| | | | AG | | |
| 149 | Human | 21 | CAAAUGAAGAAGUCUGAGA | UUUCUCAGACUUCUUCAUUUG | [2517-2537](21/21) |
| | | | AA | | |
| 150 | Human | 21 | GCAAAUGAAGAAGUCUGAG | UUCUCAGACUUCUUCAUUUGC | [2516-2536](21/21) |
| | | | AA | | |
| 151 | Human | 21 | GAAAAGCAAAUGAAGAAGU | AGACUUCUUCAUUUGCUUUUC | [2511-2531](21/21) |
| | | | CU | | |
| 152 | Human | 21 | GCAGACAAGUUACUUAAGA | ACUCUUAAGUAACUUGUCUGC | [2490-2510](21/21) |
| | | | GU | | |
| 153 | Human | 21 | GAACGCAGACAAGUUACUU | UUAAGUAACUUGUCUGCGUUC | [2486-2506](21/21) |
| | | | AA | | |
| 154 | Human | 21 | GAGAACGCAGACAAGUUAC | AAGUAACUUGUCUGCGUUCUC | [2484-2504](21/21) |
| | | | UU | | |

| | | | | | |
|---|---|---|---|---|---|
| No. | Source | length | human 41152075 | mouse 23956171 | rat 7021448 |

FOR 19
STRETCHES:

| | | | | | |
|---|---|---|---|---|---|
| 1 | Cross Sp | 19 | [3053-3071](19/19) | [3105-3123](19/19) | [3330-3348](19/19) |
| 2 | Cross Sp | 19 | [3023-3041](19/19) | [3075-3093](19/19) | [3300-3318](19/19) |
| 3 | Cross Sp | 19 | [2954-2972](19/19) | [3006-3024](19/19) | [3230-3248](19/19) |
| 4 | Cross Sp | 19 | [2452-2470](19/19) | [2504-2522](19/19) | [2726-2744](19/19) |
| 5 | Cross Sp | 19 | [3051-3069](19/19) | [3103-3121](19/19) | [3328-3346](19/19) |
| 6 | Cross Sp | 19 | [2953-2971](19/19) | [3005-3023](19/19) | [3229-3247](19/19) |
| 7 | Cross Sp | 19 | [2806-2824](19/19) | [2858-2876](19/19) | [3082-3100](19/19) |
| 8 | Cross Sp | 19 | [2802-2820](19/19) | [2854-2872](19/19) | [3078-3096](19/19) |
| 9 | Cross Sp | 19 | [2706-2724](19/19) | [2758-2776](19/19) | [2981-2999](19/19) |
| 10 | Cross Sp | 19 | [2705-2723](19/19) | [2757-2775](19/19) | [2980-2998](19/19) |
| 11 | Cross Sp | 19 | [2704-2722](19/19) | [2756-2774](19/19) | [2979-2997](19/19) |
| 12 | Cross Sp | 19 | [2703-2721](19/19) | [2755-2773](19/19) | [2978-2996](19/19) |
| 13 | Cross Sp | 19 | [2586-2604](19/19) | [2638-2656](19/19) | [2861-2879](19/19) |
| 14 | Cross Sp | 19 | [2474-2492](19/19) | [2526-2544](19/19) | [2749-2767](19/19) |
| 15 | Cross Sp | 19 | [3052-3070](19/19) | [3104-3122](19/19) | [3329-3347](19/19) |
| 16 | Cross Sp | 19 | [2347-2365](19/19) | [2399-2417](19/19) | [2621-2639](19/19) |
| 17 | Cross Sp | 19 | [3024-3042](19/19) | [3076-3094](19/19) | [3301-3319](19/19) |
| 18 | Cross Sp | 19 | [3022-3040](19/19) | [3074-3092](19/19) | [3299-3317](19/19) |
| 19 | Cross Sp | 19 | [2997-3015](19/19) | [3049-3067](19/19) | [3273-3291](19/19) |
| 20 | Cross Sp | 19 | [2956-2974](19/19) | [3008-3026](19/19) | [3232-3250](19/19) |
| 21 | Cross Sp | 19 | [2076-2094](19/19) | [2128-2146](19/19) | [2350-2368](19/19) |
| 22 | Cross Sp | 19 | [2804-2822](19/19) | [2856-2874](19/19) | [3080-3098](19/19) |
| 23 | Cross Sp | 19 | [2709-2727](19/19) | [2761-2779](19/19) | [2984-3002](19/19) |
| 24 | Cross Sp | 19 | [2587-2605](19/19) | [2639-2657](19/19) | [2862-2880](19/19) |
| 25 | Cross Sp | 19 | [1151-1169](19/19) | [1206-1224](19/19) | [1428-1446](19/19) |
| 26 | Cross Sp | 19 | [3025-3043](19/19) | [3077-3095](19/19) | [3302-3320](19/19) |
| 27 | Cross Sp | 19 | [2998-3016](19/19) | [3050-3068](19/19) | [3274-3292](19/19) |
| 28 | Cross Sp | 19 | [2805-2823](19/19) | [2857-2875](19/19) | [3081-3099](19/19) |
| 29 | Cross Sp | 19 | [2803-2821](19/19) | [2855-2873](19/19) | [3079-3097](19/19) |
| 30 | Cross Sp | 19 | [2711-2729](19/19) | [2763-2781](19/19) | [2986-3004](19/19) |
| 31 | Cross Sp | 19 | [2708-2726](19/19) | [2760-2778](19/19) | [2983-3001](19/19) |
| 32 | Cross Sp | 19 | [2558-2576](19/19) | [2610-2628](19/19) | [2833-2851](19/19) |
| 33 | Cross Sp | 19 | [2451-2469](19/19) | [2503-2521](19/19) | [2725-2743](19/19) |
| 34 | Cross Sp | 19 | [2712-2730](19/19) | [2764-2782](19/19) | [2987-3005](19/19) |
| 35 | Cross Sp | 19 | [2710-2728](19/19) | [2762-2780](19/19) | [2985-3003](19/19) |
| 36 | Cross Sp | 19 | [2707-2725](19/19) | [2759-2777](19/19) | [2982-3000](19/19) |
| 37 | Cross Sp | 19 | [2559-2577](19/19) | [2611-2629](19/19) | [2834-2852](19/19) |
| 38 | Cross Sp | 19 | [2955-2973](19/19) | [3007-3025](19/19) | [3231-3249](19/19) |
| 39 | Cross Sp | 19 | [2077-2095](19/19) | [2129-2147](19/19) | [2351-2369](19/19) |
| 40 | Cross Sp | 19 | [2713-2731](19/19) | [2765-2783](19/19) | [2988-3006](19/19) |
| 41 | Cross Sp | 19 | [2948-2966](19/19) | [3000-3018](19/19) | [3224-3242](19/19) |
| 42 | Cross Sp | 19 | [2557-2575](19/19) | [2609-2627](19/19) | [2832-2850](19/19) |
| 43 | Cross Sp | 19 | [2952-2970](19/19) | [3004-3022](19/19) | [3228-3246](19/19) |
| 44 | Cross Sp | 19 | [2950-2968](19/19) | [3002-3020](19/19) | [3226-3244](19/19) |
| 45 | Cross Sp | 19 | [2560-2578](19/19) | [2612-2630](19/19) | [2835-2853](19/19) |
| 46 | Cross Sp | 19 | [2949-2967](19/19) | [3001-3019](19/19) | [3225-3243](19/19) |
| 47 | Human | 19 | [1138-1156](19/19) | [1915-1926](12/12) | — |
| 48 | Human | 19 | [1040-1058](19/19) | — | — |
| 49 | Human | 19 | [919-937](19/19) | [974-984](11/11) | [1197-1212](15/16) |
| 50 | Human | 19 | [865-883](19/19) | — | — |
| 51 | Human | 19 | [635-653](19/19) | — | — |
| 52 | Human | 19 | [3838-3856](19/19) | — | — |
| 53 | Human | 19 | [3777-3795](19/19) | — | — |
| 54 | Human | 19 | [3597-3615](19/19) | — | — |
| 55 | Human | 19 | [3072-3090](19/19) | [3125-3142](18/18) | — |
| 56 | Human | 19 | [2907-2925](19/19) | [2959-2977](19/19) | [3183-3201](18/19) |
| 57 | Human | 19 | [2497-2515](19/19) | — | — |
| 58 | Human | 19 | [2483-2501](19/19) | [2535-2545](11/11) | — |
| 59 | Human | 19 | [2403-2421](19/19) | — | — |
| 60 | Human | 19 | [2375-2393](19/19) | [2427-2445](19/19) | [2656-2667](12/12) |
| 61 | Human | 19 | [2279-2297](19/19) | [2633-2644](12/12) | — |
| 62 | Human | 19 | [2179-2197](19/19) | — | — |
| 63 | Human | 19 | [2135-2153](19/19) | — | — |
| 64 | Human | 19 | [409-427](19/19) | — | — |
| 65 | Human | 19 | [2110-2128](19/19) | [2162-2180](19/19) | [2384-2402](18/19) |
| 66 | Human | 19 | [1970-1988](19/19) | [1920-1931](12/12) | — |
| 67 | Human | 19 | [1945-1963](19/19) | — | [2219-2237](18/19) |
| 68 | Human | 19 | [1198-1216](19/19) | — | — |
| 69 | Human | 19 | [1045-1063](19/19) | — | — |
| 70 | Human | 19 | [1021-1039](19/19) | — | — |
| 71 | Human | 19 | [1018-1036](19/19) | [1075-1091](16/17) | — |
| 72 | Human | 19 | [985-1003](19/19) | — | — |
| 73 | Human | 19 | [760-778](19/19) | [817-833](17/17) | — |
| 74 | Human | 19 | [731-749](19/19) | — | [1009-1027](18/19) |
| 75 | Human | 19 | [266-284](19/19) | — | [548-560](13/13) |
| 76 | Human | 19 | [654-672](19/19) | — | — |
| 77 | Human | 19 | [588-606](19/19) | — | — |
| 78 | Human | 19 | [3914-3932](19/19) | — | — |
| 79 | Human | 19 | [3899-3917](19/19) | — | — |
| 80 | Human | 19 | [3874-3892](19/19) | — | — |
| 81 | Human | 19 | [3860-3878](19/19) | — | — |
| 82 | Human | 19 | [3851-3869](19/19) | — | — |
| 83 | Human | 19 | [3848-3866](19/19) | — | — |
| 84 | Human | 19 | [3813-3831](19/19) | — | — |
| 85 | Human | 19 | [3792-3810](19/19) | — | — |
| 86 | Human | 19 | [3725-3743](19/19) | — | — |
| 87 | Human | 19 | [3675-3693](19/19) | — | — |
| 88 | Human | 19 | [3645-3663](19/19) | — | — |
| 89 | Human | 19 | [3641-3659](19/19) | — | — |
| 90 | Human | 19 | [3618-3636](19/19) | — | — |
| 91 | Human | 19 | [3608-3626](19/19) | — | — |
| 92 | Human | 19 | [3554-3572](19/19) | — | — |
| 93 | Human | 19 | [3529-3547](19/19) | — | — |
| 94 | Human | 19 | [3461-3479](19/19) | — | — |
| 95 | Human | 19 | [3449-3467](19/19) | — | — |
| 96 | Human | 19 | [3442-3460](19/19) | — | — |
| 97 | Human | 19 | [3439-3457](19/19) | — | — |
| 98 | Human | 19 | [3351-3369](19/19) | — | — |
| 99 | Human | 19 | [3290-3308](19/19) | — | — |
| 100 | Human | 19 | [3210-3228](19/19) | — | — |
| 101 | Human | 19 | [3182-3200](19/19) | — | — |
| 102 | Human | 19 | [3110-3128](19/19) | — | — |
| 103 | Human | 19 | [3106-3124](19/19) | — | — |
| 104 | Human | 19 | [3043-3061](19/19) | [3097-3113](16/17) | [3322-3338](16/17) |
| 105 | Human | 19 | [3029-3047](19/19) | [3081-3095](15/15) | [3306-3320](15/15) |
| 106 | Human | 19 | [2989-3007](19/19) | [3041-3059](18/19) | [3265-3283](18/19) |
| 107 | Human | 19 | [2977-2995](19/19) | [3031-3047](17/17) | [3255-3271](17/17) |
| 108 | Human | 19 | [2973-2991](19/19) | [3025-3043](18/19) | [3255-3267](13/13) |
| 109 | Human | 19 | [2966-2984](19/19) | [3018-3036](18/19) | — |
| 110 | Human | 19 | [2917-2935](19/19) | [2969-2987](18/19) | [3193-3211](18/19) |
| 111 | Human | 19 | [2888-2906](19/19) | [2944-2954](11/11) | [3168-3178](11/11) |
| 112 | Human | 19 | [2869-2887](19/19) | — | — |
| 113 | Human | 19 | [2862-2880](19/19) | — | — |
| 114 | Human | 19 | [2841-2859](19/19) | [3053-3066](14/14) | [3277-3290](14/14) |
| 115 | Human | 19 | [2826-2844](19/19) | — | — |
| 116 | Human | 19 | [2813-2831](19/19) | [2865-2883](18/19) | [3089-3107](18/19) |
| 117 | Human | 19 | [2768-2786](19/19) | — | — |
| 118 | Human | 19 | [2758-2776](19/19) | — | — |
| 119 | Human | 19 | [2755-2773](19/19) | — | — |
| 120 | Human | 19 | [2687-2705](19/19) | [1292-1302](11/11) | — |
| 121 | Human | 19 | [2666-2684](19/19) | — | — |
| 122 | Human | 19 | [2659-2677](19/19) | [2711-2726](16/16) | — |
| 123 | Human | 19 | [2656-2674](19/19) | [2708-2726](19/19) | — |
| 124 | Human | 19 | [2649-2667](19/19) | [2707-2719](13/13) | — |
| 125 | Human | 19 | [2644-2662](19/19) | — | — |
| 126 | Human | 19 | [2572-2590](19/19) | — | — |
| 127 | Human | 19 | [2567-2585](19/19) | [2619-2633](15/15) | [2842-2853](12/12) |
| 128 | Human | 19 | [2477-2495](19/19) | [2529-2545](17/17) | [2752-2767](16/16) |
| 129 | Human | 19 | [2447-2465](19/19) | [2503-2517](15/15) | [2725-2739](15/15) |
| 130 | Human | 19 | [2416-2434](19/19) | [2468-2486](18/19) | [2690-2708](18/19) |
| 131 | Human | 19 | [2410-2428](19/19) | — | — |
| 132 | Human | 19 | [2396-2414](19/19) | — | — |
| 133 | Human | 19 | [2380-2398](19/19) | [2432-2450](19/19) | [2656-2672](17/17) |
| 134 | Human | 19 | [2341-2359](19/19) | [2393-2411](18/19) | [2615-2633](19/19) |
| 135 | Human | 19 | [2335-2353](19/19) | [2387-2405](18/19) | [2609-2627](19/19) |
| 136 | Human | 19 | [2334-2352](19/19) | [2386-2404](18/19) | [2608-2626](19/19) |
| 137 | Human | 19 | [2295-2313](19/19) | — | — |
| 138 | Human | 19 | [2240-2258](19/19) | [2293-2310](17/18) | [2514-2532](18/19) |
| 139 | Human | 19 | [2237-2255](19/19) | — | — |
| 140 | Human | 19 | [2230-2248](19/19) | — | — |
| 141 | Human | 19 | [2227-2245](19/19) | — | — |
| 142 | Human | 19 | [2221-2239](19/19) | [2407-2418](12/12) | [2629-2640](12/12) |
| 143 | Human | 19 | [2168-2186](19/19) | [3064-3077](14/14) | — |

FOR 21
STRETCHES:

| | | | | | |
|---|---|---|---|---|---|
| 1 | Cross Sp | 21 | [3051-3071](21/21) | [3103-3123](21/21) | [3328-3348](21/21) |
| 2 | Cross Sp | 21 | [3023-3043](21/21) | [3075-3095](21/21) | [3300-3320](21/21) |
| 3 | Cross Sp | 21 | [2804-2824](21/21) | [2856-2876](21/21) | [3080-3100](21/21) |
| 4 | Cross Sp | 21 | [2953-2973](21/21) | [3005-3025](21/21) | [3229-3249](21/21) |
| 5 | Cross Sp | 21 | [2952-2972](21/21) | [3004-3024](21/21) | [3228-3248](21/21) |
| 6 | Cross Sp | 21 | [2802-2822](21/21) | [2854-2874](21/21) | [3078-3098](21/21) |
| 7 | Cross Sp | 21 | [2706-2726](21/21) | [2758-2778](21/21) | [2981-3001](21/21) |
| 8 | Cross Sp | 21 | [2705-2725](21/21) | [2757-2777](21/21) | [2980-3000](21/21) |
| 9 | Cross Sp | 21 | [2710-2730](21/21) | [2762-2782](21/21) | [2985-3005](21/21) |
| 10 | Cross Sp | 21 | [2709-2729](21/21) | [2761-2781](21/21) | [2984-3004](21/21) |
| 11 | Cross Sp | 21 | [2707-2727](21/21) | [2759-2779](21/21) | [2982-3002](21/21) |
| 12 | Cross Sp | 21 | [2704-2724](21/21) | [2756-2776](21/21) | [2979-2999](21/21) |
| 13 | Cross Sp | 21 | [3022-3042](21/21) | [3074-3094](21/21) | [3299-3319](21/21) |
| 14 | Cross Sp | 21 | [2708-2728](21/21) | [2760-2780](21/21) | [2983-3003](21/21) |
| 15 | Cross Sp | 21 | [2954-2974](21/21) | [3006-3026](21/21) | [3230-3250](21/21) |
| 16 | Cross Sp | 21 | [2711-2731](21/21) | [2763-2783](21/21) | [2986-3006](21/21) |
| 17 | Cross Sp | 21 | [2803-2823](21/21) | [2855-2875](21/21) | [3079-3099](21/21) |
| 18 | Cross Sp | 21 | [2950-2970](21/21) | [3002-3022](21/21) | [3226-3246](21/21) |
| 19 | Cross Sp | 21 | [2948-2968](21/21) | [3000-3020](21/21) | [3224-3244](21/21) |
| 20 | Cross Sp | 21 | [2951-2971](21/21) | [3003-3023](21/21) | [3227-3247](21/21) |
| 21 | Cross Sp | 21 | [2558-2578](21/21) | [2610-2630](21/21) | [2833-2853](21/21) |
| 22 | Cross Sp | 21 | [2949-2969](21/21) | [3001-3021](21/21) | [3225-3245](21/21) |
| 23 | Cross Sp | 21 | [2557-2577](21/21) | [2609-2629](21/21) | [2832-2852](21/21) |
| 24 | Human | 21 | [321-341](21/21) | [376-396](20/21) | — |
| 25 | Human | 21 | [208-224](17/17) | — | — |
| 26 | Human | 21 | — | [80-92](13/13) | [267-279](13/13) |
| 27 | Human | 21 | [3607-3627](21/21) | — | — |
| 28 | Human | 21 | — | — | — |
| 29 | Human | 21 | [3350-3370](21/21) | — | — |
| 30 | Human | 21 | [3159-3179](21/21) | — | [3447-3459](13/13) |
| 31 | Human | 21 | [3156-3176](21/21) | — | — |
| 32 | Human | 21 | [2906-2926](21/21) | [2958-2978](21/21) | [3182-3202](20/21) |
| 33 | Human | 21 | [2330-2350](21/21) | [2386-2402](16/17) | [2608-2624](17/17) |
| 34 | Human | 21 | [2216-2236](21/21) | [2407-2418](12/12) | [1198-1210](13/13) |
| 35 | Human | 21 | [2168-2188](21/21) | [3064-3077](14/14) | — |
| 36 | Human | 21 | [2080-2100](21/21) | [2132-2152](20/21) | [2354-2374](20/21) |
| 37 | Human | 21 | [1854-1874](21/21) | [1909-1922](14/14) | [2131-2148](18/18) |
| 38 | Human | 21 | — | — | — |
| 39 | Human | 21 | [186-206](21/21) | — | — |
| 40 | Human | 21 | [212-232](21/21) | — | — |
| 41 | Human | 21 | [208-218](11/11) | — | — |
| 42 | Human | 21 | — | — | — |
| 43 | Human | 21 | — | — | — |
| 44 | Human | 21 | — | — | — |
| 45 | Human | 21 | — | — | — |
| 46 | Human | 21 | [73-93](21/21) | — | — |
| 47 | Human | 21 | — | — | — |
| 48 | Human | 21 | — | — | — |
| 49 | Human | 21 | — | — | [350-361](12/12) |
| 50 | Human | 21 | — | [89-102](14/14) | — |
| 51 | Human | 21 | — | [80-92](13/13) | [267-279](13/13) |
| 52 | Human | 21 | — | — | — |
| 53 | Human | 21 | — | — | — |
| 54 | Human | 21 | — | [30-40](11/11) | [217-227](11/11) |
| 55 | Human | 21 | — | [22-40](19/19) | [209-227](19/19) |
| 56 | Human | 21 | — | [12-32](21/21) | [203-219](17/17) |
| 57 | Human | 21 | — | [10-29](20/20) | [197-216](19/20) |
| 58 | Human | 21 | [3907-3927](21/21) | — | — |
| 59 | Human | 21 | [3876-3896](21/21) | — | — |
| 60 | Human | 21 | [3873-3893](21/21) | — | — |
| 61 | Human | 21 | [3871-3891](21/21) | — | — |
| 62 | Human | 21 | [3843-3863](21/21) | — | — |
| 63 | Human | 21 | [3802-3822](21/21) | — | — |
| 64 | Human | 21 | [3792-3812](21/21) | — | — |
| 65 | Human | 21 | [3777-3797](21/21) | — | — |
| 66 | Human | 21 | [3769-3789](21/21) | — | — |
| 67 | Human | 21 | [3761-3781](21/21) | — | — |
| 68 | Human | 21 | [3748-3768](21/21) | — | — |
| 69 | Human | 21 | [3724-3744](21/21) | — | — |
| 70 | Human | 21 | [3674-3694](21/21) | — | — |
| 71 | Human | 21 | [3668-3688](21/21) | — | [385-395](11/11) |
| 72 | Human | 21 | [3634-3654](21/21) | — | — |
| 73 | Human | 21 | — | — | — |
| 74 | Human | 21 | [43-63](21/21) | — | — |
| 75 | Human | 21 | [3594-3614](21/21) | — | — |
| 76 | Human | 21 | [3449-3469](21/21) | — | — |
| 77 | Human | 21 | [3445-3465](21/21) | — | — |
| 78 | Human | 21 | [3347-3367](21/21) | — | — |
| 79 | Human | 21 | [40-60](21/21) | — | — |
| 80 | Human | 21 | [3289-3309](21/21) | — | [3564-3574](11/11) |
| 81 | Human | 21 | [3286-3306](21/21) | — | — |
| 82 | Human | 21 | — | — | — |
| 83 | Human | 21 | [3209-3229](21/21) | — | — |
| 84 | Human | 21 | — | — | — |
| 85 | Human | 21 | — | — | — |
| 86 | Human | 21 | [3133-3153](21/21) | — | — |
| 87 | Human | 21 | [3125-3145](21/21) | — | — |
| 88 | Human | 21 | — | — | — |
| 89 | Human | 21 | [3110-3130](21/21) | — | — |
| 90 | Human | 21 | [3105-3125](21/21) | — | — |
| 91 | Human | 21 | — | — | — |
| 92 | Human | 21 | [3020-3040](21/21) | [3072-3092](21/21) | [3297-3317](21/21) |
| 93 | Human | 21 | [3000-3020](21/21) | [3052-3068](17/17) | [3276-3292](17/17) |
| 94 | Human | 21 | — | — | — |
| 95 | Human | 21 | [2872-2892](21/21) | — | — |
| 96 | Human | 21 | [2862-2882](21/21) | — | — |
| 97 | Human | 21 | [2835-2855](21/21) | — | — |
| 98 | Human | 21 | [2820-2840](21/21) | — | — |
| 99 | Human | 21 | [2812-2832](21/21) | [2864-2884](20/21) | [3088-3108](20/21) |
| 100 | Human | 21 | [2801-2821](21/21) | [2854-2873](20/20) | [3078-3097](20/20) |
| 101 | Human | 21 | [2799-2819](21/21) | [2854-2871](18/18) | [3078-3095](18/18) |
| 102 | Human | 21 | [2757-2777](21/21) | — | — |
| 103 | Human | 21 | [2754-2774](21/21) | — | — |
| 104 | Human | 21 | — | — | — |
| 105 | Human | 21 | [2751-2771](21/21) | — | — |
| 106 | Human | 21 | [2700-2720](21/21) | [2755-2772](18/18) | [2978-2995](18/18) |
| 107 | Human | 21 | — | — | — |
| 108 | Human | 21 | [2648-2668](21/21) | [2707-2720](14/14) | — |
| 109 | Human | 21 | [2643-2663](21/21) | — | — |
| 110 | Human | 21 | [2514-2534](21/21) | [2566-2586](21/21) | [2789-2809](20/21) |
| 111 | Human | 21 | [2447-2467](21/21) | [2503-2519](17/17) | [2725-2741](17/17) |
| 112 | Human | 21 | [2424-2444](21/21) | [2476-2486](11/11) | [2698-2718](21/21) |
| 113 | Human | 21 | [2406-2426](21/21) | — | — |
| 114 | Human | 21 | [2391-2411](21/21) | — | — |
| 115 | Human | 21 | [2379-2399](21/21) | [2431-2450](20/20) | [2656-2672](17/17) |
| 116 | Human | 21 | [2349-2369](21/21) | [2401-2417](17/17) | [2623-2639](17/17) |
| 117 | Human | 21 | [2338-2358](21/21) | [2390-2410](20/21) | [2612-2632](21/21) |
| 118 | Human | 21 | [2334-2354](21/21) | [2386-2406](20/21) | [2608-2628](21/21) |
| 119 | Human | 21 | [2328-2348](21/21) | [2380-2397](17/18) | [2602-2622](20/21) |
| 120 | Human | 21 | [2312-2332](21/21) | [2364-2384](20/21) | [2590-2606](16/17) |
| 121 | Human | 21 | [1197-1217](21/21) | — | — |
| 122 | Human | 21 | [1134-1154](21/21) | [1915-1926](12/12) | — |
| 123 | Human | 21 | [1039-1059](21/21) | — | — |
| 124 | Human | 21 | [981-1001](21/21) | [1036-1055](19/20) | — |
| 125 | Human | 21 | [771-791](21/21) | [826-846](21/21) | [1049-1069](20/21) |
| 126 | Human | 21 | [759-779](21/21) | [817-834](18/18) | — |
| 127 | Human | 21 | [758-778](21/21) | [817-833](17/17) | — |
| 128 | Human | 21 | [658-678](21/21) | — | — |
| 129 | Human | 21 | [644-666](21/21) | [701-721](20/21) | [924-944](20/21) |
| 130 | Human | 21 | [634-654](21/21) | — | — |
| 131 | Human | 21 | [587-607](21/21) | — | — |
| 132 | Human | 21 | [558-578](21/21) | [614-633](18/20) | — |
| 133 | Human | 21 | [2307-2327](21/21) | [2359-2378](20/20) | [2581-2600](19/20) |
| 134 | Human | 21 | [2272-2292](21/21) | [2326-2344](18/19) | [2548-2558](11/11) |
| 135 | Human | 21 | [2238-2258](21/21) | [2293-2310](17/18) | [2512-2532](20/21) |
| 136 | Human | 21 | [2219-2239](21/21) | [2407-2418](12/12) | [1198-1210](13/13) |
| 137 | Human | 21 | [2167-2187](21/21) | [3064-3077](14/14) | — |
| 138 | Human | 21 | [2111-2131](21/21) | [2163-2183](21/21) | [2385-2405](20/21) |
| 139 | Human | 21 | [2085-2105](21/21) | [2137-2156](19/20) | [2359-2378](19/20) |
| 140 | Human | 21 | [2068-2088](21/21) | [2128-2140](13/13) | [2350-2362](13/13) |
| 141 | Human | 21 | [2029-2049](21/21) | [2089-2101](13/13) | — |
| 142 | Human | 21 | [2027-2047](21/21) | [2089-2099](11/11) | — |
| 143 | Human | 21 | [2006-2026](21/21) | — | — |
| 144 | Human | 21 | [1992-2012](21/21) | [3061-3071](11/11) | [3285-3295](11/11) |
| 145 | Human | 21 | [1990-2010](21/21) | [3061-3071](11/11) | [3285-3295](11/11) |
| 146 | Human | 21 | [1959-1979](21/21) | [1918-1929](12/12) | [2233-2252](18/20) |
| 147 | Human | 21 | [1955-1975](21/21) | — | [2229-2249](19/21) |
| 148 | Human | 21 | [1950-1970](21/21) | — | [2227-2239](13/13) |
| 149 | Human | 21 | [1920-1940](21/21) | [2401-2411](11/11) | [2194-2214](19/21) |
| 150 | Human | 21 | [1919-1939](21/21) | [2401-2411](11/11) | [2193-2213](19/21) |
| 151 | Human | 21 | [1914-1934](21/21) | [2401-2411](11/11) | [2188-2204](17/17) |
| 152 | Human | 21 | [1893-1913](21/21) | [1947-1965](18/19) | [2168-2180](13/13) |
| 153 | Human | 21 | [1889-1909](21/21) | [1947-1958](12/12) | [2168-2180](13/13) |
| 154 | Human | 21 | [1887-1907](21/21) | [1947-1958](12/12) | [2168-2180](13/13) |

Table M below shows further siRNA compounds specific for the SYNPO2L gene that have been selected according to the present invention.

TABLE M

| | | | | | |
|---|---|---|---|---|---|
| | | Oligo | | | |
| No | Source | Length | Sense siRNA | AntiSense siRNA | human 50301231 |

FOR 19
STRETCHES:

| | | | | | |
|---|---|---|---|---|---|
| 1 | Cross Sp | 19 | AGACGAAGAACUCGCCCAA | UUGGGCGAGUUCUUCGUCU | [1278-1296](19/19) |
| 2 | Cross Sp | 19 | CUAUUGCUUACAACCCACU | AGUGGGUUGUAAGCAAUAG | [1779-1797](19/19) |
| 3 | Cross Sp | 19 | GAGCCAAGAAGUACACCCU | AGGGUGUACUUCUUGGCUC | [261-279](19/19) |
| 4 | Cross Sp | 19 | CCUCCUAUUGCUUACAACC | GGUUGUAAGCAAUAGGAGG | [1775-1793](19/19) |
| 5 | Cross Sp | 19 | CCUAUUGCUUACAACCCAC | GUGGGUUGUAAGCAAUAGG | [1778-1796](19/19) |
| 6 | Cross Sp | 19 | UCCUAUUGCUUACAACCCA | UGGGUUGUAAGCAAUAGGA | [1777-1795](19/19) |
| 7 | Cross Sp | 19 | CUCCUAUUGCUUACAACCC | GGGUUGUAAGCAAUAGGAG | [1776-1794](19/19) |
| 8 | Cross Sp | 19 | GAGACGAAGAACUCGCCCA | UGGGCGAGUUCUUCGUCUC | [1277-1295](19/19) |
| 9 | Cross Sp | 19 | ACGAAGAACUCGCCCAACC | GGUUGGGCGAGUUCUUCGU | [1280-1298](19/19) |
| 10 | Cross Sp | 19 | GACGAAGAACUCGCCCAAC | GUUGGGCGAGUUCUUCGUC | [1279-1297](19/19) |
| 11 | Cross Sp | 19 | AGAGAGCCAAGAAGUACAC | GUGUACUUCUUGGCUCUCU | [258-276](19/19) |
| 12 | Cross Sp | 19 | CAGAGAGCCAAGAAGUACA | UGUACUUCUUGGCUCUCUG | [257-275](19/19) |
| 13 | Cross Sp | 19 | GGCAGAGAGCCAAGAAGUA | UACUUCUUGGCUCUCUGCC | [255-273](19/19) |
| 14 | Cross Sp | 19 | UCUUUGAACAGCAGCGCCA | UGGCGCUGCUGUUCAAAGA | [519-537](19/19) |
| 15 | Cross Sp | 19 | AGAGCCAAGAAGUACACCC | GGGUGUACUUCUUGGCUCU | [260-278](19/19) |
| 16 | Cross Sp | 19 | GCAGAGAGCCAAGAAGUAC | GUACUUCUUGGCUCUCUGC | [256-274](19/19) |
| 17 | Cross Sp | 19 | GAGAGCCAAGAAGUACACC | GGUGUACUUCUUGGCUCUC | [259-277](19/19) |
| 18 | Cross Sp | 19 | AGCUCUUUGAACAGCAGCG | CGCUGCUGUUCAAAGAGCU | [516-534](19/19) |
| 19 | Cross Sp | 19 | CAGCUCUUUGAACAGCAGC | GCUGCUGUUCAAAGAGCUG | [515-533](19/19) |
| 20 | Cross Sp | 19 | CGGCAGAGAGCCAAGAAGU | ACUUCUUGGCUCUCUGCCG | [254-272](19/19) |
| 21 | Cross Sp | 19 | GCUCUUUGAACAGCAGCGC | GCGCUGCUGUUCAAAGAGC | [517-535](19/19) |
| 22 | Cross Sp | 19 | CUCUUUGAACAGCAGCGCC | GGCGCUGCUGUUCAAAGAG | [518-536](19/19) |
| 23 | Human | 19 | CCAGCUCCAAGCAUCUUUA | UAAAGAUGCUUGGAGCUGG | [725-743](19/19) |
| 24 | Human | 19 | GGACGAAGAAGCCUUCUCU | AGAGAAGGCUUCUUCGUCC | [355-373](19/19) |
| 25 | Human | 19 | CCACAGAUCUCUCUUCCUU | AAGGAAGAGAGAUCUGUGG | [2568-2586](19/19) |
| 26 | Human | 19 | GGAACAUCCAGUUCCUAAA | UUUAGGAACUGGAUGUUCC | [2316-2334](19/19) |
| 27 | Human | 19 | CCACUGCUCUCUCCCUUUU | AAAAGGGAGAGAGCAGUGG | [1793-1811](19/19) |
| 28 | Human | 19 | CUACCACCUCGGUUAUUUU | AAAAUAACCGAGGUGGUAG | [792-810](19/19) |
| 29 | Human | 19 | CAACUACCACCUCGGUUAU | AUAACCGAGGUGGUAGUUG | [789-807](19/19) |
| 30 | Human | 19 | CCAACUACCACCUCGGUUA | UAACCGAGGUGGUAGUUGG | [788-806](19/19) |
| 31 | Human | 19 | GGGUGCAGCUCUUUGAACA | UGUUCAAAGAGCUGCACCC | [510-528](19/19) |
| 32 | Human | 19 | CGCAGCCUCACCAAUCAAU | AUUGAUUGGUGAGGCUGCG | [380-398](19/19) |
| 33 | Human | 19 | CAAAAAGUUAGCAACUUGA | UCAAGUUGCUAACUUUUUG | [3421-3439](19/19) |
| 34 | Human | 19 | CUCAGGAUUUCAAAAAGUU | AACUUUUUGAAAUCCUGAG | [3411-3429](19/19) |
| 35 | Human | 19 | CUAUACCUCAGGAUUUCAA | UUGAAAUCCUGAGGUAUAG | [3405-3423](19/19) |
| 36 | Human | 19 | CAUGGGCUGAGUUAUACCA | UGGUAUAACUCAGCCCAUG | [3359-3377](19/19) |
| 37 | Human | 19 | UGAAGGGCCAGGACAGUUU | AAACUGUCCUGGCCCUUCA | [3339-3357](19/19) |
| 38 | Human | 19 | CAGGUGUACAGCAUCUUGU | ACAAGAUGCUGUACACCUG | [3296-3314](19/19) |
| 39 | Human | 19 | GAGAAUAACUCCUUAGAGA | UCUCUAAGGAGUUAUUCUC | [3264-3282](19/19) |
| 40 | Human | 19 | GAAAGAGAUUCACUAGAGA | UCUCUAGUGAAUCUCUUUC | [3249-3267](19/19) |
| 41 | Human | 19 | GGGAGAAAGAGAUUCACUA | UAGUGAAUCUCUUUCUCCC | [3245-3263](19/19) |
| 42 | Human | 19 | UGAGUCUUAUCGUUGUACU | AGUACAACGAUAAGACUCA | [3190-3208](19/19) |
| 43 | Human | 19 | CAAUCUGAGUCUUAUCGUU | AACGAUAAGACUCAGAUUG | [3185-3203](19/19) |
| 44 | Human | 19 | GACUCUGUCUCAAAACAAA | UUUGUUUUGAGACAGAGUC | [3149-3167](19/19) |
| 45 | Human | 19 | GAGACUCUGUCUCAAAACA | UGUUUUGAGACAGAGUCUC | [3147-3165](19/19) |
| 46 | Human | 19 | GCGAGACUCUGUCUCAAAA | UUUUGAGACAGAGUCUCGC | [3145-3163](19/19) |
| 47 | Human | 19 | CUUGCAGUGAGCUGAGAUU | AAUCUCAGCUCACUGCAAG | [3096-3114](19/19) |
| 48 | Human | 19 | GCAGGAGAAUGGCAUGAAC | GUUCAUGCCAUUCUCCUGC | [3064-3082](19/19) |
| 49 | Human | 19 | CCCAUUUCCACUAAAAAUA | UAUUUUUAGUGGAAAUGGG | [2985-3003](19/19) |
| 50 | Human | 19 | GAGGGGAGGUCAAAAUAAA | UUUAUUUUGACCUCCCCUC | [2848-2866](19/19) |
| 51 | Human | 19 | UAAGAGGGGAGGUCAAAAU | AUUUUGACCUCCCCUCUUA | [2845-2863](19/19) |
| 52 | Human | 19 | CCAAGCUUGACUGGCAUAU | AUAUGCCAGUCAAGCUUGG | [2756-2774](19/19) |
| 53 | Human | 19 | UCAGUGAGAAGUAAACAGA | UCUGUUUACUUCUCACUGA | [2625-2643](19/19) |
| 54 | Human | 19 | CCCUGGGCCUCAUUUUAAU | AUUAAAAUGAGGCCCAGGG | [8 2604-2622](19/19) |
| 55 | Human | 19 | GAUCUCUCUUCCUUGACAU | AUGUCAAGGAAGAGAGAUC | [2573-2591](19/19) |
| 56 | Human | 19 | CCAUAUUCCUCCACAGAUC | GAUCUGUGGAGGAAUAUGG | [2558-2576](19/19) |
| 57 | Human | 19 | GGAUCUCUGUCUUUAUUUC | GAAAUAAAGACAGAGAUCC | [2526-2544](19/19) |
| 58 | Human | 19 | CUGGAUCUCUGUCUUUAUU | AAUAAAGACAGAGAUCCAG | [2524-2542](19/19) |
| 59 | Human | 19 | CUCUCUUUGUCUCUGCAUU | AAUGCAGAGACAAAGAGAG | [2498-2516](19/19) |
| 60 | Human | 19 | CCAAGAAGUACACCCUGGU | ACCAGGGUGUACUUCUUGG | [264-282](19/19) |
| 61 | Human | 19 | CCUUCCUCCUCACUCUCUA | UAGAGAGUGAGGAGGAAGG | [2432-2450](19/19) |
| 62 | Human | 19 | GAGAUGGUUUCCAAGUUGA | UCAACUUGGAAACCAUCUC | [2398-2416](19/19) |
| 63 | Human | 19 | GCAUCUGGAAGCUAAAUUG | CAAUUUAGCUUCCAGAUGC | [2369-2387](19/19) |
| 64 | Human | 19 | UCACGCAUCUGGAAGCUAA | UUAGCUUCCAGAUGCGUGA | [2365-2383](19/19) |
| 65 | Human | 19 | ACAUCCAGUUCCUAAAGUU | AACUUUAGGAACUGGAUGU | [2319-2337](19/19) |
| 66 | Human | 19 | GGAUUGCAAGCUCAUGUGU | ACACAUGAGCUUGCAAUCC | [2240-2258](19/19) |
| 67 | Human | 19 | GAACAGGAUUGCAAGCUCA | UGAGCUUGCAAUCCUGUUC | [2235-2253](19/19) |
| 68 | Human | 19 | CACCAGAACAGGAUUGCAA | UUGCAAUCCUGUUCUGGUG | [2230-2248](19/19) |
| 69 | Human | 19 | GGGUACUUAUGUUUAAGAA | UUCUUAAACAUAAGUACCC | [231-249](19/19) |
| 70 | Human | 19 | CAAAGGGGUACUUAUGUUU | AAACAUAAGUACCCCUUUG | [226-244](19/19) |
| 71 | Human | 19 | CCAAAGGGGUACUUAUGUU | AACAUAAGUACCCCUUUGG | [225-243](19/19) |
| 72 | Human | 19 | CCACUCCAAAGGGGUACUU | AAGUACCCCUUUGGAGUGG | [220-238](19/19) |
| 73 | Human | 19 | AAACUGCCAUGUUCUGUUU | AAACAGAACAUGGCAGUUU | [1923-1941](19/19) |
| 74 | Human | 19 | GCCCUAUCAACUUAAAACU | AGUUUUAAGUUGAUAGGGC | [1909-1927](19/19) |
| 75 | Human | 19 | UCAGCCCUAUCAACUUAAA | UUUAAGUUGAUAGGGCUGA | [1906-1924](19/19) |
| 76 | Human | 19 | CAAGGCUCUAGAUUUUAUG | CAUAAAAUCUAGAGCCUUG | [1882-1900](19/19) |
| 77 | Human | 19 | CAUCAAGGCUCUAGAUUUU | AAAAUCUAGAGCCUUGAUG | [1879-1897](19/19) |
| 78 | Human | 19 | CCCCCUUCCUGGAAAUAUU | AAUAUUUCCAGGAAGGGGG | [1736-1754](19/19) |
| 79 | Human | 19 | GUGGGGAGCUGUUUGCUAA | UUAGCAAACAGCUCCCCAC | [1623-1641](19/19) |
| 80 | Human | 19 | CCCUAUGACUCCUAAGACU | AGUCUUAGGAGUCAUAGGG | [1495-1513](19/19) |
| 81 | Human | 19 | CCCCCUAUGACUCCUAAGA | UCUUAGGAGUCAUAGGGGG | [1493-1511](19/19) |
| 82 | Human | 19 | CCUCACGUGACACCUAAGA | UCUUAGGUGUCACGUGAGG | [1445-1463](19/19) |
| 83 | Human | 19 | GAGGCCAAGACCAAAUGCA | UGCAUUUGGUCUUGGCCUC | [164-182](19/19) |
| 84 | Human | 19 | CCAGGAGUUACAAGACCCU | AGGGUCUUGUAACUCCUGG | [1425-1443](19/19) |
| 85 | Human | 19 | GCAACUUCAUGCAGCCAGU | ACUGGCUGCAUGAAGUUGC | [1401-1419](19/19) |
| 86 | Human | 19 | GCUGAAGCCUGCAACUUCA | UGAAGUUGCAGGCUUCAGC | [1391-1409](19/19) |
| 87 | Human | 19 | AGAAUCUGGUCCUGAAGAA | UUCUUCAGGACCAGAUUCU | [1354-1372](19/19) |
| 88 | Human | 19 | GCAUAAAAGAGGCCAAGAC | GUCUUGGCCUCUUUUAUGC | [156-174](19/19) |
| 89 | Human | 19 | GAACCUGGAUGAAAAGCCU | AGGCUUUUCAUCCAGGUUC | [1321-1339](19/19) |
| 90 | Human | 19 | GAAAGGAGGAGACGAAGAA | UUCUUCGUCUCCUCCUUUC | [1269-1287](19/19) |
| 91 | Human | 19 | CAAGAGAAGAGCAUAAAAG | CUUUUAUGCUCUUCUCUUG | [146-164](19/19) |
| 92 | Human | 19 | CAGAGAGCCUCCAAGAGAA | UUCUCUUGGAGGCUCUCUG | [135-153](19/19) |
| 93 | Human | 19 | GUUCUAUGCAGAACUGCAA | UUGCAGUUCUGCAUAGAAC | [112-130](19/19) |
| 94 | Human | 19 | CUCGUUCUAUGCAGAACUG | CAGUUCUGCAUAGAACGAG | [109-127](19/19) |
| 95 | Human | 19 | CAAGACUCGUUCUAUGCAG | CUGCAUAGAACGAGUCUUG | [104-122](19/19) |
| 96 | Human | 19 | CCAAGACUCGUUCUAUGCA | UGCAUAGAACGAGUCUUGG | [103-121](19/19) |
| 97 | Human | 19 | CUCCAAGACUCGUUCUAUG | CAUAGAACGAGUCUUGGAG | [101-119](19/19) |
| 98 | Human | 19 | CCACCUCGGUUAUUUUCCG | CGGAAAAUAACCGAGGUGG | [795-813](19/19) |
| 99 | Human | 19 | AGCUCCAAGACUCGUUCUA | UAGAACGAGUCUUGGAGCU | [99-117](19/19) |
| 100 | Human | 19 | UGAGCUCCAAGACUCGUUC | GAACGAGUCUUGGAGCUCA | [97-115](19/19) |
| 101 | Human | 19 | CCAAGCAUCUUUAACCGGU | CCUGUUAAAGAUGCUUGG | [731-749](19/19) |
| 102 | Human | 19 | CUCCAAGCAUCUUUAACCG | CGGUUAAAGAUGCUUGGAG | [729-747](19/19) |
| 103 | Human | 19 | CAGCUCCAAGCAUCUUUAA | UUAAAGAUGCUUGGAGCUG | [726-744](19/19) |
| 104 | Human | 19 | CCCAGCUCCAAGCAUCUUU | AAAGAUGCUUGGAGCUGGG | [724-742](19/19) |
| 105 | Human | 19 | CAGUUCCUGAGCUCCAAGA | UCUUGUAGCUCAGGAACUG | [90-108](19/19) |
| 106 | Human | 19 | GCAGCUCUUUGAACAGCAG | CUGCUGUUCAAAGAGCUGC | [514-532](19/19) |
| 107 | Human | 19 | AGACCUUUUAGCCCAUCAG | CUGAUGGGCUCAAAGGUCU | [27-45](19/19) |
| 108 | Human | 19 | CCUAUCUGGACAUGGAGCU | AGCUCCAUGUCCAGAUAGG | [414-432](19/19) |
| 109 | Human | 19 | GUCCCUAUCUGGACAUGGA | UCCAUGUCCAGAUAGGGAC | [411-429](19/19) |
| 110 | Human | 19 | CCAAGCCAGCUAUGACAAA | UUUGUCAUAGCUGGCUUGG | [61-79](19/19) |
| 111 | Human | 19 | GCCAAGCCAGCUAUGACAA | UUGUCAUAGCUGGCUUGGC | [60-78](19/19) |
| 112 | Human | 19 | CCUCACCAAUCAAUCUGAC | GUCAGAUUGAUUGGUGAGG | [385-403](19/19) |
| 113 | Human | 19 | GCCUCACCAAUCAAUCUGA | UCAGAUUGAUUGGUGAGGC | [384-402](19/19) |
| 114 | Human | 19 | AGCCAAGCCAGCUAUGACA | UGUCAUAGCUGGCUUGGCU | [59-77](19/19) |
| 115 | Human | 19 | GCAGCCUCACCAAUCAAUC | GAUUGAUUGGUGAGGCUGC | [381-399](19/19) |
| 116 | Human | 19 | GGAUUUCAAAAAGUUAGCA | UGCUAACUUUUUGAAAUCC | [3415-3433](19/19) |
| 117 | Human | 19 | CCUCAGGAUUUCAAAAAGU | ACUUUUUGAAAUCCUGAGG | [3410-3428](19/19) |
| 118 | Human | 19 | GAAUUCUAUACCUCAGGAU | AUCCUGAGGUAUAGAAUUC | [3400-3418](19/19) |
| 119 | Human | 19 | ACAAAGAAUUCUAUACCUC | GAGGUAUAGAAUUCUUUGU | [3395-3413](19/19) |
| 120 | Human | 19 | GAACAAAGAAUUCUAUACC | GGUAUAGAAUUCUUUGUUC | [3393-3411](19/19) |
| 121 | Human | 19 | CAGGAAUAGAACAAAGAAU | AUUCUUUGUUCUAUUCCUG | [3385-3403](19/19) |
| 122 | Human | 19 | CCAGGAAUAGAACAAAGAA | UUCUUUGUUCUAUUCCUGG | [3384-3402](19/19) |

FOR 21
STRETCHES:

| | | | | | |
|---|---|---|---|---|---|
| 1 | Cross Sp | 21 | UCCUAUUGCUUACAACCCACU | AGUGGGUUGUAAGCAAUAGGA | [1777-1797](21/21) |
| 2 | Cross Sp | 21 | CUCCUAUUGCUUACAACCCAC | GUGGGUUGUAAGCAAUAGGAU | [1776-1796](21/21) |
| 3 | Cross Sp | 21 | CCUCCUAUUGCUUACAACCCA | UGGGUUGUAAGCAAUAGGAGG | [1775-1795](21/21) |
| 4 | Cross Sp | 21 | GAGACGAAGAACUCGCCCAAC | GUUGGGCGAGUUCUUCGUCUC | [1277-1297](21/21) |
| 5 | Cross Sp | 21 | AGACGAAGAACUCGCCCAACC | GGUUGGGCGAGUUCUUCGUCU | [1278-1298](21/21) |
| 6 | Cross Sp | 21 | CAGAGAGCCAAGAAGUACACC | GGUGUACUUCUUGGCUCUCUG | [257-277](21/21) |
| 7 | Cross Sp | 21 | GCAGAGAGCCAAGAAGUACAC | GUGUACUUCUUGGCUCUCUGC | [256-276](21/21) |
| 8 | Cross Sp | 21 | GAGAGCCAAGAAGUACACCCU | AGGGUGUGUACUUCUUGGCUCUC | [259-279](21/21) |
| 9 | Cross Sp | 21 | GGCAGAGAGCCAAGAAGUACA | UGUACUUCUUGGCUCUCUGCC | [255-275](21/21) |
| 10 | Cross Sp | 21 | AGAGAGCCAAGAAGUACACCC | GGGUGUACUUCUUGGCUCUCU | [258-278](21/21) |
| 11 | Cross Sp | 21 | GCGGCAGAGAGCCAAGAAGUA | UACUUCUUGGCUCUCUGCCGC | [253-273](21/21) |
| 12 | Cross Sp | 21 | CGGCAGAGAGCCAAGAAGUAC | GUACUUCUUGGCUCUCUGCCG | [254-274](21/21) |
| 13 | Cross Sp | 21 | CAGCUCUUUGAACAGCAGCAGCGC | GCGCUGCUGUUCAAAGAGCUG | [515-535](21/21) |
| 14 | Cross Sp | 21 | GCUCUUUGAACAGCAGCAGCGCCA | UGGCGCUGCUGUUCAAAGAGC | [517-537](21/21) |
| 15 | Cross Sp | 21 | AGCUCUUUGAACAGCAGCGCC | GGCGCUGCUGUUCAAAGAGCU | [516-536](21/21) |
| 16 | Human | 21 | CGAGACUCUGUCUCAAAACAA | UUGUUUUGAGACAGAGUCUCG | [3146-3166](21/21) |
| 17 | Human | 21 | CAAGUAUGUCUGCUUUGGUAU | AUACCAAAGCAGACAUACUUG | [2471-2491](21/21) |
| 18 | Human | 21 | CCAAAGGGGUACUUAUGUUUA | UAAACAUAAGUACCCCUUUGG | [225-245](21/21) |
| 19 | Human | 21 | GCAUCAAGGCUCUAGAUUUUA | UAAAAUCUAGAGCCUUGAUGC | [1878-1898](21/21) |
| 20 | Human | 21 | CAAGACCAAAUGCAGGACAAU | AUUGUCCUGCAUUUGGUCUUG | [169-189](21/21) |
| 21 | Human | 21 | CCAAGAGAAGAGCAUAAAAGA | UCUUUUAUGCUCUUCUCUUGG | [145-165](21/21) |
| 22 | Human | 21 | CAAGACUCGUUCUAUGCAGAA | UUCUGCAUAGAACGAGUCUUG | [104-124](21/21) |
| 23 | Human | 21 | CCAACUACCACCUCGGUUAUU | AAUAACCGAGGUGGUAGUUGG | [788-808](21/21) |
| 24 | Human | 21 | GCCAACUACCACCUCGGUUAU | AUAACCGAGGUGGUAGUUGGC | [787-807](21/21) |
| 25 | Human | 21 | GAGCUCCAAGACUCGUUCUAU | AUAGAACGAGUCUUGGAGCUC | [98-118](21/21) |
| 26 | Human | 21 | CCCAGCUCCAAGCAUCUUUAA | UUAAAGAUGCUUGGAGCUGGG | [724-744](21/21) |
| 27 | Human | 21 | UGACUGGGACAGUCCCUAUCU | AGAUAGGGACUGUCCCAGUCA | [400-420](21/21) |
| 28 | Human | 21 | CCAAUCAAUCUGACUGGGACA | UGUCCCAGUCAGAUUGAUUGG | [390-410](21/21) |
| 29 | Human | 21 | CAGCCAAGCCAGCUAUGACAA | UUGUCAUAGCUGGCUUGGCUG | [58-78](21/21) |
| 30 | Human | 21 | CCUCAGGAUUUCAAAAAGUUA | UAACUUUUUGAAAUCCUGAGG | [3410-3430](21/21) |
| 31 | Human | 21 | AGAAUUCUAUACCUCAGGAUU | AAUCCUGAGGUAUAGAAUUCU | [3399-3419](21/21) |
| 32 | Human | 21 | CCAGGAAUAGAACAAAGAAUU | AAUUCUUUGUUCUAUUCCUGG | [3384-3404](21/21) |
| 33 | Human | 21 | GACCAGGAAUAGAACAAAGAA | UUCUUUGUUCUAUUCCUGGUC | [3382-3402](21/21) |
| 34 | Human | 21 | CUAGACCAGGAAUAGAACAAA | UUUGUUCUAUUCCUGGUCUAG | [3379-3399](21/21) |
| 35 | Human | 21 | GCAUGGGCUGAGUUAUACCAG | CUGGUAUAACUCAGCCCAUGC | [3358-3378](21/21) |
| 36 | Human | 21 | GUGAAAGUUAUGGAUCAUGAA | UUCAUGCUCCAUAACUUUCAC | [3313-3333](21/21) |
| 37 | Human | 21 | GUACAGCAUCUUGUGAAAGUU | AACUUUCACAAGAUGCUGUAC | [3301-3321](21/21) |
| 38 | Human | 21 | GCAGGUGUACAGCAUCUUGUG | CACAAGAUGCUGUACACCUGC | [3295-3315](21/21) |
| 39 | Human | 21 | GUAGCAGGUGUACAGCAUCUU | AAGAUGCUGUACACCUGCUAC | [3292-3312](21/21) |
| 40 | Human | 21 | CUAGAGAAUAACUCCUUAGAG | CUCUAAGGAGUUAUUCUCUAG | [3261-3281](21/21) |
| 41 | Human | 21 | AGAAAGAGAUUCACUAGAGAA | UUCUCUAGUGAAUCUCUUUCU | [3248-3268](21/21) |
| 42 | Human | 21 | GUUGAGUGGGAGAAAGAGAUU | AAUCUCUUUCUCCCACUCAAC | [3238-3258](21/21) |
| 43 | Human | 21 | CCAAUCUGAGUCUUAUCGUUG | CAACGAUAAGACUCAGAUUGG | [3184-3204](21/21) |
| 44 | Human | 21 | CAAACAAAAGACCCAAUCUGA | UCAGAUUGGGUCUUUUGUUUG | [3172-3192](21/21) |
| 45 | Human | 21 | CUGUCUCAAAACAAACAAACA | UGUUUGUUUGUUUUGAGACAG | [3153-3172](21/21) |
| 46 | Human | 21 | GGAGCGAGACUCUGUCUCAAA | UUUGAGACAGAGUCUCGCUCC | [3142-3162](21/21) |
| 47 | Human | 21 | AAACCCCAUUUCCACUAAAAA | UUUUUAGUGGAAAUGGGGUUU | [2981-3001](21/21) |
| 48 | Human | 21 | CAAAAUAAAGACCCAAUCUGA | UCAGAUUGGGUCUUUAUUUUG | [2858-2878](21/21) |
| 49 | Human | 21 | GUCAAAAUAAAGACCCAAUCU | AGAUUGGGUCUUUAUUUUGAC | [2856-2876](21/21) |
| 50 | Human | 21 | CUUCCGUCUAAGGCAUGAAUA | UAUUCAUGCCUUAGACGGAAG | [2826-2846](21/21) |
| 51 | Human | 21 | GCAUAUACCUAGGCCUGUGUG | CACACAGGCCUAGGUAUAUGC | [2769-2789](21/21) |
| 52 | Human | 21 | CAAGCUUGACUGGCAUAUACC | GGUAUAUGCCAGUCAAGCUUG | [2757-2777](21/21) |
| 53 | Human | 21 | CCACUCUGCUUUCCCAAGCUU | AAGCUUGGGAAAGCAGAGUGG | [2743-2763](21/21) |
| 54 | Human | 21 | AGAUCUCUCUUCCUUGACAUU | AAUGUCAAGGAAGAGAGAUCU | [2572-2392](21/21) |
| 55 | Human | 21 | CCCAUAUUCCUCCACAGAUCU | AGAUCUGUGGAGGAAUAUGGG | [2557-2577](21/21) |
| 56 | Human | 21 | CCACCCAUAUUCCUCCACAGA | UCUGUGGAGGAAUAUGGGUGG | [2554-2574](21/21) |
| 57 | Human | 21 | CCAGGCUUCUCCACCCAUAUU | AAUAUGGGUGGAGAAGCCUGG | [2544-2564](21/21) |
| 58 | Human | 21 | CCUGGAUCUCUGUCUUUAUUU | AAAUAAAGACAGAGAUCCAGG | [2523-2543](21/21) |
| 59 | Human | 21 | GCAUUUCCUUUCCUGGAUCUC | GAGAUCCAGGAAAGGAAAUGC | [2512-2532](21/21) |
| 60 | Human | 21 | CCUCUCUUUGUCUCUGCAUUU | AAAUGCAGAGACAAAGAGAGG | [2497-2517](21/21) |
| 61 | Human | 21 | CAACCAAGUAUGUCUGCUUUG | CAAAGCAGACAUACUUGGUUG | [2467-2487](21/21) |
| 62 | Human | 21 | CCAACCAAGUAUGUCUGCUUU | AAAGCAGACAUACUUGGUUGG | [2466-2486](21/21) |
| 63 | Human | 21 | CCGCUUUCCAACAAGUAUGU | ACAUACUUGGUUGGAAAGCGG | [2459-2479](21/21) |
| 64 | Human | 21 | AGAGAUGGUUUCCAAGUUGAU | AUCAACUUGGAAACCAUCUCU | [2397-2417](21/21) |
| 65 | Human | 21 | GUCACGCAUCUGGAAGCUAAA | UUUAGCUUCCAGAUGCGUGAC | [2364-2384](21/21) |
| 66 | Human | 21 | GAACAUCCAGUUCCUAAAGUU | AACUUUAGGAACUGGAUGUUC | [2317-2337](21/21) |
| 67 | Human | 21 | CAAGGAGAGGUGGAACAUCCA | UGGAUGUUCCACCUCUCCUUG | [2305-2325](21/21) |
| 68 | Human | 21 | GACCAAGGAGAGGUGGAACAU | AUGUUCCACCUCUCCUUGGUC | [2302-2322](21/21) |
| 69 | Human | 21 | CAGGAUUGCAAGCUCAUGUGU | ACACAUGAGCUUGCAAUCCUG | [2238-2258](21/21) |
| 70 | Human | 21 | CCACCAGAACAGGAUUGCAAG | CUUGCAAUCCUGUUCUGGUGG | [2229-2249](21/21) |
| 71 | Human | 21 | CGAUUUCAGCCACCAGAACA | UGUUCUGGUGUCUGAAAAUCG | [2219-2239](21/21) |
| 72 | Human | 21 | GCCCAUCUGGAGAACAGAACU | AGUUCUGUUCUCCAGAUGGGC | [2101-2121](21/21) |
| 73 | Human | 21 | UGAGCCCAUCUGGAGAACAGA | UCUGUUCUCCAGAUGGGCUCA | [2098-2118](21/21) |
| 74 | Human | 21 | GGGGUACUUAUGUUUAAGAAA | UUUCUUAAACAUAAGUACCCC | [230-250](21/21) |
| 75 | Human | 21 | GCAUCAGCCCUAUCAACUUAA | UUAAGUUGAUAGGGCUGAUGC | [1903-1923](21/21) |
| 76 | Human | 21 | CAGGGCAUCAAGGCUCUAGAU | AUCUAGAGCCUUGAUGCCCUG | [1874-1894](21/21) |
| 77 | Human | 21 | CCCACUGCUCUCUCCCUUUUU | AAAAAGGGAGAGAGCAGUGGG | [1792-1812](21/21) |
| 78 | Human | 21 | GGAAAUAUUCACCCAACAUCC | GGAUGUUGGGUGAAUAUUUCC | [1746-1766](21/21) |
| 79 | Human | 21 | CUGGAAAUAUUCACCCAACAU | AUGUUGGGUGAAUAUUUCCAG | [1744-1764](21/21) |
| 80 | Human | 21 | GAAGCCUGCAACUUCAUGCAG | CUGCAUGAAGUUGCAGGCUUC | [1394-1414](21/21) |
| 81 | Human | 21 | GGCUGAAGCCUGCAACUUCAU | AUGAAGUUGCAGGCUUCAGCC | [1390-1410](21/21) |
| 82 | Human | 21 | GUACAGAACCUGGAUGAAAAG | CUUUUCAUCCAGGUUCUGUAC | [1316-1336](21/21) |
| 83 | Human | 21 | CUGGUACAGAACCUGGAUGAA | UUCAUCCAGGUUCUGUACCAG | [1313-1333](21/21) |
| 84 | Human | 21 | GGAAAGGAGGAGACGAAGAAC | GUUCUUCGUCUCCUCCUUUCC | [1268-1288](21/21) |
| 85 | Human | 21 | CCACCGCUUCUAUCUUCCUAU | AUAGGAAGAUAGAAGCGGUGG | [1083-1103](21/21) |
| 86 | Human | 21 | CAGAACUGCAACGUGCAGAGA | UCUCUGCACGUUGCAGUUCUG | [120-140](21/21) |
| 87 | Human | 21 | CGUUCUAUGCAGAACUGCAAC | GUUGCAGUUCUGCAUAGAACG | [111-131](21/21) |
| 88 | Human | 21 | CCAAGACUCGUUCUAUGCAGA | UCUGCAUAGAACGAGUCUUGG | [103-123](21/21) |
| 89 | Human | 21 | CUCCAAGACUCGUUCUAUGCA | UGCAUAGAACGAGUCUUGGAG | [101-121](21/21) |
| 90 | Human | 21 | GCUCCAAGACUCGUUCUAUGC | GCAUAGAACGAGUCUUGGAGC | [100-120](21/21) |
| 91 | Human | 21 | CUACCACCUCGGUUAUUUUCC | GGAAAAUAACCGAGGUGGUAG | [792-812](21/21) |
| 92 | Human | 21 | CUGAGCUCCAAGACUCGUUCU | AGAACGAGUCUUGGAGCUCAG | [96-116](21/21) |
| 93 | Human | 21 | CCAAGCAUCUUUAACCGGUCA | UGACCGGUUAAAGAUGCUUGG | [731-751](21/21) |
| 94 | Human | 21 | CCAGCUCCAAGCAUCUUUAAC | GUUAAAGAUGCUUGGAGCUGG | [725-745](21/21) |
| 95 | Human | 21 | CCCCAGCUCCAAGCAUCUUUA | UAAAGAUGCUUGGAGCUGGGG | [723-743](21/21) |
| 96 | Human | 21 | CAGUUCCUGAGCUCCAAGACU | AGUCUUGGAGCUCAGGAACUG | [90-110](21/21) |
| 97 | Human | 21 | CCCUAUCUGGACAUGGAGCUU | AAGCUCCAUGUCCAGAUAGGG | [413-433](21/21) |
| 98 | Human | 21 | CAAGCCAGCUAUGACAAAGCC | GGCUUUGUCAUAGCUGGCUUG | [62-82](21/21) |
| 99 | Human | 21 | GGACAGUCCCUAUCUGGACAU | AUGUCCAGAUAGGGACUGUCC | [406-426](21/21) |
| 100 | Human | 21 | GCCUCACCAAUCAAUCUGACU | AGUCAGAUUGAUUGGUGAGGC | [384-404](21/21) |
| 101 | Human | 21 | CAGCCUCACCAAUCAAUCUGA | UCAGAUUGAUUGGUGAGGCUG | [382-402](21/21) |
| 102 | Human | 21 | CGCAGCCUCACCAAUCAAUCU | AGAUUGAUUGGUGAGGCUGCG | [380-400](21/21) |
| 103 | Human | 21 | CAAAAAGUUAGCAACUUGAGA | UCUCAAGUUGCUAACUUUUUG | [3421-3441](21/21) |
| 104 | Human | 21 | UCAAAAAGUUAGCAACUUGAG | CUCAAGUUGCUAACUUUUUGA | [3420-3440](21/21) |
| 105 | Human | 21 | GGAUUUCAAAAAGUUAGCAAC | GUUGCUAACUUUUUGAAAUCC | [3415-3435](21/21) |
| 106 | Human | 21 | CAGGAUUUCAAAAAGUUAGCA | UGCUAACUUUUUGAAAUCCUG | [3413-3433](21/21) |
| 107 | Human | 21 | CUAUACCUCAGGAUUUCAAAA | UUUUGAAAUCCUGAGGUAUAG | [3405-3425](21/21) |
| 108 | Human | 21 | UCUAUACCUCAGGAUUCAAA | UUUGAAAUCCUGAGGUAUAGA | [3404-3424](21/21) |
| 109 | Human | 21 | GAAUUCUAUACCUCAGGAUUU | AAAUCCUGAGGUAUAGAAUUC | [3400-3420](21/21) |
| 110 | Human | 21 | GAACAAAGAAUUCUAUACCUC | GAGGUAUAGAAUUCUUUGUUC | [3393-3413](21/21) |
| 111 | Human | 21 | CAGGAAUAGAACAAAGAAUUC | GAAUUCUUUGUUCUAUUCCUG | [3385-3405](21/21) |
| 112 | Human | 21 | AGACCAGGAAUAGAACAAAGA | UCUUUGUUCUAUUCCUGGUCU | [3381-3401](21/21) |
| 113 | Human | 21 | GGACGAAGAAGCCUUCUCUGA | UCAGAGAAGGCUUCUUCGUCC | [355-375](21/21) |
| 114 | Human | 21 | CAGCUAGACCAGGAAUAGAAC | GUUCUAUUCCUGGUCUAGCUG | [3376-3396](21/21) |
| 115 | Human | 21 | GCUGAGUUAUACCAGCUAGAC | GUCUAGCUGGUAUAACUCAGC | [3364-3384](21/21) |

| | | | | |
|---|---|---|---|---|
| | | Oligo | | |
| No | Source | Length | mouse 31341052 | rat 34868957 |

FOR 19
STRETCHES:

| | | | | |
|---|---|---|---|---|
| 1 | Cross Sp | 19 | [2030-2048](19/19) | [2246-2264](19/19) |
| 2 | Cross Sp | 19 | [2537-2555](19/19) | [2753-2771](19/19) |
| 3 | Cross Sp | 19 | [1013-1031](19/19) | [1229-1247](19/19) |
| 4 | Cross Sp | 19 | [2533-2551](19/19) | [2749-2767](19/19) |
| 5 | Cross Sp | 19 | [2536-2554](19/19) | [2752-2770](19/19) |
| 6 | Cross Sp | 19 | [2535-2553](19/19) | [2751-2769](19/19) |
| 7 | Cross Sp | 19 | [2534-2552](19/19) | [2750-2768](19/19) |
| 8 | Cross Sp | 19 | [2029-2047](19/19) | [2245-2263](19/19) |
| 9 | Cross Sp | 19 | [2032-2050](19/19) | [2248-2266](19/19) |
| 10 | Cross Sp | 19 | [2031-2049](19/19) | [2247-2265](19/19) |
| 11 | Cross Sp | 19 | [1010-1028](19/19) | [1226-1244](19/19) |
| 12 | Cross Sp | 19 | [1009-1027](19/19) | [1225-1243](19/19) |
| 13 | Cross Sp | 19 | [1007-1025](19/19) | [1223-1241](19/19) |
| 14 | Cross Sp | 19 | [1271-1289](19/19) | [1490-1508](19/19) |
| 15 | Cross Sp | 19 | [1012-1030](19/19) | [1228-1246](19/19) |
| 16 | Cross Sp | 19 | [1008-1026](19/19) | [1224-1242](19/19) |
| 17 | Cross Sp | 19 | [1011-1029](19/19) | [1227-1245](19/19) |
| 18 | Cross Sp | 19 | [1268-1286](19/19) | [1487-1505](19/19) |
| 19 | Cross Sp | 19 | [1267-1285](19/19) | [1486-1504](19/19) |
| 20 | Cross Sp | 19 | [1006-1024](19/19) | [1222-1240](19/19) |
| 21 | Cross Sp | 19 | [1269-1287](19/19) | [1488-1506](19/19) |
| 22 | Cross Sp | 19 | [1270-1288](19/19) | [1489-1507](19/19) |
| 23 | Human | 19 | — | — |
| 24 | Human | 19 | — | — |
| 25 | Human | 19 | — | — |
| 26 | Human | 19 | — | — |
| 27 | Human | 19 | — | — |
| 28 | Human | 19 | — | — |
| 29 | Human | 19 | — | — |
| 30 | Human | 19 | — | — |
| 31 | Human | 19 | [1262-1280](18/19) | [1481-1499](18/19) |
| 32 | Human | 19 | — | — |
| 33 | Human | 19 | [3875-3885](11/11) | — |
| 34 | Human | 19 | — | — |
| 35 | Human | 19 | — | — |
| 36 | Human | 19 | [3815-3825](11/11) | — |
| 37 | Human | 19 | — | — |
| 38 | Human | 19 | [3754-3765](12/12) | — |
| 39 | Human | 19 | — | — |
| 40 | Human | 19 | — | — |
| 41 | Human | 19 | — | — |
| 42 | Human | 19 | — | — |
| 43 | Human | 19 | — | — |
| 44 | Human | 19 | — | — |
| 45 | Human | 19 | — | — |
| 46 | Human | 19 | — | — |
| 47 | Human | 19 | — | — |
| 48 | Human | 19 | — | — |
| 49 | Human | 19 | — | — |
| 50 | Human | 19 | — | — |
| 51 | Human | 19 | — | — |
| 52 | Human | 19 | — | — |
| 53 | Human | 19 | — | — |
| 54 | Human | 19 | — | — |
| 55 | Human | 19 | — | — |
| 56 | Human | 19 | — | — |
| 57 | Human | 19 | — | — |
| 58 | Human | 19 | — | — |
| 59 | Human | 19 | — | — |
| 60 | Human | 19 | [1016-1031](16/16) | [1232/1247](16/16) |
| 61 | Human | 19 | [3434-3445](12/12) | — |
| 62 | Human | 19 | [2952-2963](12/12) | — |
| 63 | Human | 19 | — | [3966-3976](11/11) |
| 64 | Human | 19 | — | [3966-3976](11/11) |
| 65 | Human | 19 | — | — |
| 66 | Human | 19 | — | — |
| 67 | Human | 19 | — | — |
| 68 | Human | 19 | [2985-3002](17/18) | — |
| 69 | Human | 19 | [983-1001](18/19) | [1199-1217](18/19) |
| 70 | Human | 19 | — | — |
| 71 | Human | 19 | — | — |
| 72 | Human | 19 | — | — |
| 73 | Human | 19 | — | [2900-2918](18/19) |
| 74 | Human | 19 | [2677-2687](11/11) | [2893-2904](12/12) |
| 75 | Human | 19 | — | — |
| 76 | Human | 19 | — | — |
| 77 | Human | 19 | — | — |
| 78 | Human | 19 | [2494-2510)(16/17) | [2710-2726](16/17) |
| 79 | Human | 19 | — | — |
| 80 | Human | 19 | — | — |
| 81 | Human | 19 | — | [2461-2479](18/19) |
| 82 | Human | 19 | — | — |
| 83 | Human | 19 | — | [1132-1149](18/18) |
| 84 | Human | 19 | [2178-2193](16/16) | [2394-2411](18/18) |
| 85 | Human | 19 | 2155-2169](15/15) | [2371-2385](15/15) |
| 86 | Human | 19 | [2143-2161](18/19) | — |
| 87 | Human | 19 | [2107-2117](11/11) | [2323-2333](11/11) |
| 88 | Human | 19 | — | [1132-1142](11/11) |
| 89 | Human | 19 | [2073-2087](15/15) | [2289-2303](15/15) |
| 90 | Human | 19 | [2023-2039](16/17) | [2237-2255](18/19) |
| 91 | Human | 19 | — | [1114-1124](11/11) |
| 92 | Human | 19 | — | [1108-1121](14/14) |
| 93 | Human | 19 | — | [1080-1098](19/19) |
| 94 | Human | 19 | — | [1079-1095](17/17) |
| 95 | Human | 19 | — | [1072-1090](18/19) |
| 96 | Human | 19 | — | [1072-1089](17/18) |
| 97 | Human | 19 | — | — |
| 98 | Human | 19 | — | — |
| 99 | Human | 19 | — | — |
| 100 | Human | 19 | — | — |
| 101 | Human | 19 | — | — |
| 102 | Human | 19 | — | — |
| 103 | Human | 19 | — | — |
| 104 | Human | 19 | — | [3680-3690](11/11) |
| 105 | Human | 19 | — | [2561-2572](12/12) |
| 106 | Human | 19 | [1267-1284](18/18) | [1486-1503](18/18) |
| 107 | Human | 19 | — | — |
| 108 | Human | 19 | [1166-1184](18/19) | [1387-1403](16/17) |
| 109 | Human | 19 | — | — |
| 110 | Human | 19 | — | [1035-1053](18/19) |
| 111 | Human | 19 | — | [1034-1052](18/19) |
| 112 | Human | 19 | — | — |
| 113 | Human | 19 | — | — |
| 114 | Human | 19 | — | [1033-1051](18/19) |
| 115 | Human | 19 | — | — |
| 116 | Human | 19 | — | — |
| 117 | Human | 19 | — | — |
| 118 | Human | 19 | — | — |
| 119 | Human | 19 | [3848-3858](11/11) | — |
| 120 | Human | 19 | [3848-3858](11/11) | — |
| 121 | Human | 19 | — | — |
| 122 | Human | 19 | — | — |

| FOR 21 | | | | |
|---|---|---|---|---|
| STRETCHES: | | | | |
| 1 | Cross Sp | 21 | (2535-2555)(21/21) | [2751-2771](21/21) |
| 2 | Cross Sp | 21 | [2534-2554](21/21) | [2750-2770](21/21) |
| 3 | Cross Sp | 21 | [2533-2553](21/21) | [2749-2769](21/21) |
| 4 | Cross Sp | 21 | [2029-2049](21/21) | [2245-2265](21/21) |
| 5 | Cross Sp | 21 | [2030-2050](21/21) | [2246-2266](21/21) |
| 6 | Cross Sp | 21 | [1009-1029](21/21) | [1225-1245](21/21) |
| 7 | Cross Sp | 21 | [1008-1028](21/21) | [1224-1244](21/21) |
| 8 | Cross Sp | 21 | [1011-1031](21/21) | [1227-1247](21/21) |
| 9 | Cross Sp | 21 | [1007-1027](21/21) | [1223-1243](21/21) |
| 10 | Cross Sp | 21 | [1010-1030](21/21) | [1226-1246](21/21) |
| 11 | Cross Sp | 21 | [1005-1025](21/21) | [1221-1241](21/21) |
| 12 | Cross Sp | 21 | [1006-1026](21/21) | [1222-1242](21/21) |
| 13 | Cross Sp | 21 | [1267-1287](21/21) | [1486-1506](21/21) |
| 14 | Cross Sp | 21 | [1269-1289](21/21) | [1488-1508](21/21) |
| 15 | Cross Sp | 21 | [1268-1288](21/21) | [1487-1507](21/21) |
| 16 | Human | 21 | — | — |
| 17 | Human | 21 | — | — |
| 18 | Human | 21 | — | — |
| 19 | Human | 21 | — | — |
| 20 | Human | 21 | — | [1137-1154](17/18) |
| 21 | Human | 21 | — | [1113-1124](21/21) |
| 22 | Human | 21 | — | [1072-1092](20/21) |
| 23 | Human | 21 | — | — |
| 24 | Human | 21 | — | — |
| 25 | Human | 21 | — | — |
| 26 | Human | 21 | — | [3680-3690](11/11) |
| 27 | Human | 21 | [1153-1172](19/20) | [1372-1391](18/20) |
| 28 | Human | 21 | — | — |
| 29 | Human | 21 | — | [1032-1052](20/21) |
| 30 | Human | 21 | — | — |
| 31 | Human | 21 | — | — |
| 32 | Human | 21 | — | — |
| 33 | Human | 21 | — | — |
| 34 | Human | 21 | — | — |
| 35 | Human | 21 | [3815-3825](11/11) | — |
| 36 | Human | 21 | — | — |
| 37 | Human | 21 | [3754-3769](16/16) | — |
| 38 | Human | 21 | [3754-3766](13/13) | — |
| 39 | Human | 21 | — | — |
| 40 | Human | 21 | — | — |
| 41 | Human | 21 | — | — |
| 42 | Human | 21 | — | — |
| 43 | Human | 21 | — | — |
| 44 | Human | 21 | — | — |
| 45 | Human | 21 | — | — |
| 46 | Human | 21 | — | — |
| 47 | Human | 21 | — | — |
| 48 | Human | 21 | — | — |
| 49 | Human | 21 | — | — |
| 50 | Human | 21 | — | — |
| 51 | Human | 21 | — | — |
| 52 | Human | 21 | — | — |
| 53 | Human | 21 | — | — |
| 54 | Human | 21 | — | — |
| 55 | Human | 21 | — | — |
| 56 | Human | 21 | — | — |
| 57 | Human | 21 | [3248-3261](14/14) | — |
| 58 | Human | 21 | — | — |
| 59 | Human | 21 | — | — |
| 60 | Human | 21 | — | — |
| 61 | Human | 21 | — | — |
| 62 | Human | 21 | — | — |
| 63 | Human | 21 | — | — |
| 64 | Human | 21 | — | — |
| 65 | Human | 21 | — | — |
| 66 | Human | 21 | — | — |
| 67 | Human | 21 | — | — |
| 68 | Human | 21 | — | — |
| 69 | Human | 21 | — | — |
| 70 | Human | 21 | — | — |
| 71 | Human | 21 | — | — |
| 72 | Human | 21 | — | — |
| 73 | Human | 21 | — | — |
| 74 | Human | 21 | [982-1001](19/20) | [1198-1217](19/20) |
| 75 | Human | 21 | — | — |
| 76 | Human | 21 | — | — |
| 77 | Human | 21 | — | — |
| 78 | Human | 21 | [2504-2524](20/21) | [2720-2740](20/21) |
| 79 | Human | 21 | [2502-2522](20/21) | [2718-2738](20/21) |
| 80 | Human | 21 | [2146-2166](20/21) | [2362-2382](20/21) |
| 81 | Human | 21 | [2142-2162](20/21) | — |
| 82 | Human | 21 | [2071-2087](17/17) | [2284-2303](20/20) |
| 83 | Human | 21 | [2071-2085](15/15) | [2281-2301](21/21) |
| 84 | Human | 21 | [2023-2040](17/18) | [2236-2256](20/21) |
| 85 | Human | 21 | — | — |
| 86 | Human | 21 | — | [1088-1100](13/13) |
| 87 | Human | 21 | — | [1079-1099](21/21) |
| 88 | Human | 21 | — | [1072-1091](19/20) |
| 89 | Human | 21 | — | [1072-1089](17/18) |
| 90 | Human | 21 | — | — |
| 91 | Human | 21 | — | — |
| 92 | Human | 21 | — | — |
| 93 | Human | 21 | — | — |
| 94 | Human | 21 | — | — |
| 95 | Human | 21 | — | [3680-3691](12/12) |
| 96 | Human | 21 | — | [2561-2572](12/12) |
| 97 | Human | 21 | [1165-1184](19/20) | [1387-1403](16/17) |
| 98 | Human | 21 | — | — |
| 99 | Human | 21 | — | — |
| 100 | Human | 21 | — | — |
| 101 | Human | 21 | — | — |
| 102 | Human | 21 | — | — |
| 103 | Human | 21 | [3875-3885](11/11) | — |
| 104 | Human | 21 | [3875-3885](11/11) | — |
| 105 | Human | 21 | [3875-3885](11/11) | — |
| 106 | Human | 21 | — | — |
| 107 | Human | 21 | — | — |
| 108 | Human | 21 | — | — |
| 109 | Human | 21 | — | — |
| 110 | Human | 21 | [3848-3858](11/11) | — |
| 111 | Human | 21 | — | — |
| 112 | Human | 21 | — | — |
| 113 | Human | 21 | — | — |
| 114 | Human | 21 | — | — |
| 115 | Human | 21 | [3815-3834](19/20) | — |

### Example 2

### Further Cardiovascular-Related Genes

Further preferred cardiovascular-related genes according to the present invention, including their GeneBank Reference numbers, are listed in Table N below. A comprehensive list of cardiovascular-related genes according to the present invention including their GeneBank Reference numbers, are listed in Table O.

TABLE N

| No. | Description | Acc | Gi | Symbol |
|---|---|---|---|---|
| | Homo sapiens coiled-coil domain containing 80 (CCDC80), transcript | NM_199511 | 41152073 | CCDC80 |
| | variant 1, mRNA | | | (SSG1) |
| | Homo sapiens synaptopodin 2 (SYNPO2), mRNA | NM_133477 | 55741765 | SYNPO2 |
| 61 | Homo sapiens von Willebrand factor (VWF), mRNA | NM_000552 | 9257255 | VWF |
| 62 | Homo sapiens hypothetical protein FLJ11078 (FLJ11078), mRNA | NM_018316 | 8922853 | FLJ11078 |
| 77 | Homo sapiens KIAA0265 protein (KIAA0265), mRNA | NM_014997 | 66348151 | KIAA0265 |
| 101 | Homo sapiens olfactomedin-like 3 (OLFML3), mRNA | NM_020190 | 50593011 | OLFML3 |
| 121 | Homo sapiens KIAA0182 protein (KIAA0182), mRNA | NM_014615 | 44955925 | KIAA0182 |
| 136 | Homo sapiens KIAA1533 (KIAA1533), mRNA | NM_020895 | 34147343 | KIAA1533 |
| 170 | Homo sapiens cDNA FLJ31748 fis, clone NT2RI2007384, highly similar | AK056310 | 16551675 | |
| | to Homo sapiens mixed lineage kinase mRNA | | | |
| 181 | Homo sapiens cDNA FLJ14791 fis, clone NT2RP4001064, weakly | AK027697 | 14042570 | |
| | similar to SYNAPTONEMAL COMPLEX PROTEIN SC65 | | | |
| 184 | Homo sapiens interferon induced transmembrane protein 2 (1-8D) | NM_006435 | 10835237 | IFITM2 |
| | (IFITM2), mRNA | | | |
| 205 | Homo sapiens mRNA for KIAA0774 protein, partial cds | AB018317 | 71891682 | |
| 218 | Homo sapiens cDNA FLJ10319 fis, clone NT2RM2000490, weakly | AK001181 | 7022274 | |
| | similar to SYNAPTOTAGMIN | | | |
| 289 | Homo sapiens tissue inhibitor of metalloproteinase 2 (TIMP2), mRNA | NM_003255 | 52632388 | TIMP2 |
| 295 | Homo sapiens S100 calcium binding protein A6 (calcyclin) (S100A6), | NM_014624 | 52352807 | S100A6 |
| | mRNA | | | |
| 330 | full-length cDNA clone CL0BA006ZC10 of Placenta of Homo sapiens | CR602328 | 50483135 | |
| | (human) | | | |
| 351 | Homo sapiens secreted protein, acidic, cysteine-rich (osteonectin) | NM_003118 | 48675809 | SPARC |
| | (SPARC), mRNA | | | |
| 359 | Homo sapiens fibronectin 1 (FN1), transcript variant 1, mRNA | NM_212482 | 47132556 | FN1 |
| 369 | Homo sapiens KIAA0355 (KIAA0355), mRNA | NM_014686 | 46276867 | KIAA0355 |
| 371 | Homo sapiens insulin-like growth factor binding protein 5 (IGFBP5), | NM_000599 | 46094066 | IGFBP5 |
| | mRNA | | | |
| 405 | Homo sapiens heparin-binding EGF-like growth factor (HBEGF), mRNA | NM_001945 | 4503412 | HBEGF |
| 415 | PREDICTED: Homo sapiens KIAA1458 protein (KIAA1458), mRNA | XM_044434 | 42657086 | KIAA1458 |
| 433 | Homo sapiens steroid sensitive gene 1 (URB), transcript variant 2, | NM_199512 | 41152075 | URB |
| | mRNA | | | |
| 478 | Homo sapiens cDNA FLJ44539 fis, clone UTERU3005205 | AK126503 | 34533004 | |
| 479 | Homo sapiens cDNA FLJ42531 fis, clone BRACE3002508, highly | AK124522 | 34530327 | WDR11 |
| | similar to Homo sapiens WD repeat domain 11 (WDR11) | | | |
| 480 | Homo sapiens cDNA FLJ16619 fis, clone TESTI4014553, highly similar | AK122930 | 34528375 | |
| | to Homo sapiens high-risk human papilloma viruses E6 oncoproteins | | | |
| | targeted protein E6TP1 alpha | | | |
| 496 | Homo sapiens serpin peptidase inhibitor, clade E (nexin, plasminogen | NM_006216 | 34147323 | SERPINE2 |
| | activator inhibitor type 1), member 2 (SERPINE2), mRNA | | | |
| 538 | Homo sapiens metallothionein 1K (MT1K), mRNA | NM_176870 | 28866965 | MT1K |
| 561 | Homo sapiens cDNA FLJ90785 fis, clone THYRO1001457, moderately | AK075266 | 22761241 | |
| | similar to H. sapiens mRNA for protein kinase C mu | | | |
| 575 | Homo sapiens cDNA FLJ37984 fis, clone CTONG2010652, weakly | R617323 | 21754536 | |
| | similar to THREONINE SYNTHASE (EC 4.2.99.2) | | | |
| 581 | Homo sapiens AXL receptor tyrosine kinase (AXL), transcript variant 1, | NM_021913 | 21536465 | AXL |
| | mRNA | | | |
| 596 | Homo sapiens c-fos induced growth factor (vascular endothelial growth | NM_004469 | 19924297 | FIGF |
| | factor D) (FIGF), mRNA | | | |
| 612 | Homo sapiens GNAS complex locus (GNAS), transcript variant 3, | NM_080425 | 18428897 | GNAS |
| | mRNA | | | |
| 629 | Homo sapiens cDNA FLJ25275 fis, clone STM06043 | AK058004 | 16554002 | |
| 650 | Homo sapiens hypothetical protein FLJ11301 (FLJ11301), mRNA | NM_018385 | 14149719 | FLJ11301 |
| 670 | Homo sapiens small muscle protein, X-linked (SMPX), mRNA | NM_014332 | 10047089 | SMPX |
| 696 | Homo sapiens hypothetical protein FLJ11046 (FLJ11046), mRNA | NM_018309 | 8922641 | FLJ11046 |
| 712 | Homo sapiens mRNA for KIAA1533 protein, partial cds | AB040966 | 7959332 | |
| 722 | Homo sapiens tumor necrosis factor receptor superfamily, member 12A | NM_016639 | 7706185 | TNFRSF12A |
| | (TNFRSF12A), mRNA | | | |
| 726 | Homo sapiens KIAA0494 (KIAA0494), mRNA | NM_014774 | 7662159 | KIAA0494 |
| 743 | Homo sapiens leukemia inhibitory factor receptor (LiFR), mRNA | NM_002310 | 73486667 | LIFR |
| 746 | Homo sapiens procollagen C-endopeptidase enhancer (PCOLCE), | NM_002593 | 7262388 | PCOLCE |
| | mRNA | | | |
| 851 | Homo sapiens vimentin (VIM), mRNA | NM_003380 | 62414288 | VIM |
| 884 | Homo sapiens granulin (GRN), transcript variant 2, mRNA | NM_001012479 | 60498994 | GRN |
| 885 | Homo sapiens elastin microfibril interfacer 2 (EMILIN2), mRNA | NM_032048 | 60498977 | EMILIN2 |
| 916 | Homo sapiens serine/threonine kinase 11 (Peutz-Jeghers syndrome) | NM_000455 | 58530881 | STK11 |
| | (STK11), mRNA | | | |
| 939 | Homo sapiens IQ motif containing GTPase activating protein 1 | NM_003870 | 57242794 | IQGAP1 |
| | (IQGAP1), mRNA | | | |
| 940 | Homo sapiens KIAA0100 gene product (KIAA0100), mRNA | NM_014680 | 57242773 | KIAA0100 |
| 961 | Homo sapiens transmembrane protein 64 (TMEM64), mRNA | NM_001008495 | 56606065 | TMEM64 |
| 962 | Homo sapiens similar to RIKEN cDNA 2310016C16 (LOC493869), | NM_001008397 | 56605999 | LOC493869 |
| | mRNA | | | |
| 988 | Homo sapiens KIAA1815 (KIAA1815), mRNA | NM_024896 | 55749803 | KIAA1815 |
| 1004 | Homo sapiens mRNA for KIAA0638 splice variant 2 | AB177859 | 54633201 | |
| 1043 | Homo sapiens transmembrane protein 30A (TMEM30A), mRNA | NM_018247 | 52694667 | TMEM30A |
| 1137 | full-length cDNA clone CS0DI077YN15 of Placenta Cot 25-normalized | CR615454 | 50496261 | |
| | of Homo sapiens (human) | | | |
| 1163 | full-length cDNA clone CS0DJ005YD11 of T cells (Jurkat cell line) Cot | CR603000 | 50483807 | |
| | 10-normalized of Homo sapiens (human) | | | |
| 1234 | Homo sapiens matrix metalloproteinase 9 (gelatinase B, 92 kDa | NM_004994 | 4826835 | MMP9 |
| | gelatinase, 92 kDa type IV collagenase) (MMP9), mRNA | | | |
| 1267 | Homo sapiens decorin (DCN), transcript variant A2, mRNA | NM_133503 | 47419926 | DCN |
| 1271 | Homo sapiens protein kinase C, delta (PRKCD), transcript variant 2, | NM_212539 | 47157324 | PRKCD |
| | mRNA | | | |
| 1274 | Homo sapiens protein kinase, cAMP-dependent, regulatory, type I, | NM_002734 | 47132579 | PRKAR1A |
| | alpha (tissue specific extinguisher 1) (PRKAR1A), transcript variant 1, | | | |
| | mRNA | | | |
| 1282 | Homo sapiens cDNA FLJ16643 fis, clone TESTI4029528 | AK131475 | 47077464 | |
| 1338 | Homo sapiens growth arrest-specific 6 (GAS6), mRNA | NM_000820 | 4557616 | GAS6 |
| 1379 | Homo sapiens spermidine/spermine N1-acetyltransferase (SAT), | NM_002970 | 4506788 | SAT |
| | mRNA | | | |
| 1389 | Homo sapiens ornithine decarboxylase 1 (ODC1), mRNA | NM_002539 | 4505488 | ODC1 |
| 1393 | Homo sapiens macrophage migration inhibitory factor (glycosylation- | NM_002415 | 4505184 | MIF |
| | inhibiting factor) (MIF), mRNA | | | |
| 1396 | Homo sapiens insulin-like growth factor binding protein 7 (IGFBP7), | NM_001553 | 4504618 | IGFBP7 |
| | mRNA | | | |
| 1408 | Homo sapiens epithelial membrane protein 3 (EMP3), mRNA | NM_001425 | 4503562 | EMP3 |
| 1416 | Homo sapiens connective tissue growth factor (CTGF), mRNA | NM_001901 | 4503122 | CTGF |
| 1436 | Homo sapiens KIAA0152 (KIAA0152), mRNA | NM_014730 | 44662824 | KIAA0152 |
| 1440 | Homo sapiens p21/Cdc42/Rac1-activated kinase 1 (STE20 homolog, | NM_002576 | 42794768 | PAK1 |
| | yeast) (PAK1), mRNA | | | |
| 1523 | Homo sapiens thrombospondin 1 (THBS1), mRNA | NM_003246 | 40317625 | THBS1 |
| 1533 | Homo sapiens sphingosine-1-phosphate phosphatase 1 (SGPP1), | NM_030791 | 40254975 | SGPP1 |
| | mRNA | | | |
| 1540 | Homo sapiens natriuretic peptide receptor A/guanylate cyclase A | NM_000906 | 40254425 | NPR1 |
| | (atrionatriuretic peptide receptor A) (NPR1), mRNA | | | |
| 1567 | Homo sapiens mRNA for KIAA0768 protein, partial cds | AB018311 | 3882256 | |
| 1577 | Homo sapiens KIAA1244 (KIAA1244), mRNA | NM_020340 | 38569486 | KIAA1244 |
| 1626 | PREDICTED: Homo sapiens FLJ00133 protein (FLJ00133), mRNA | XM_059482 | 37549556 | FLJ00133 |
| 1646 | Homo sapiens fibulin 1 (FBLN1), transcript variant D, mRNA | NM_006486 | 34734065 | FBLN1 |
| 1647 | Homo sapiens fibulin 1 (FBLN1), transcript variant C, mRNA | NM_001996 | 34734061 | FBLN1 |
| 1654 | Homo sapiens cDNA FLJ45929 fis, clone PLACE7000266, weakly | AK127826 | 34534907 | TTN |
| | similar to Homo sapiens titin (TTN), transcript variant N2-B | | | |
| 1664 | Homo sapiens cDNA FLJ43306 fis, clone NT2NE2006909, highly | AK125296 | 34531345 | |
| | similar to Methionine aminopeptidase 2 (EC 3.4.11.18) | | | |
| 1667 | Homo sapiens cDNA FLJ41022 fis, clone ASTRO1000009, weakly | AK123018 | 34528466 | |
| | similar to BAND 4.1-LIKE PROTEIN 4 | | | |
| 1698 | Homo sapiens biglycan (BGN), mRNA | NM_001711 | 34304351 | BGN |
| 1705 | Homo sapiens cysteine-rich, angiogenic inducer, 61 (CYR61), mRNA | NM_001554 | 34222286 | CYR61 |
| 1738 | Homo sapiens SM-11044 binding protein (SMBP), mRNA | NM_020123 | 33859832 | SMBP |
| 1748 | Homo sapiens phospholipase C, gamma 1 (PLCG1), transcript variant | NM_182811 | 33598945 | PLCG1 |
| | 2, mRNA | | | |
| 1773 | Homo sapiens protein kinase, AMP-activated, gamma 2 non-catalytic | NM_016203 | 33186924 | PRKAG2 |
| | subunit (PRKAG2), mRNA | | | |
| 1805 | Homo sapiens serine palmitoyltransferase, long chain base subunit 2 | NM_004863 | 31881646 | SPTLC2 |
| | (SPTLC2), mRNA | | | |
| 1818 | Homo sapiens pim-1 oncogene (PIM1), mRNA | NM_002648 | 31543400 | PIM1 |
| 1822 | Homo sapiens metallothionein 2A (MT2A), mRNA | NM_005953 | 31543214 | MT2A |
| 1893 | Homo sapiens reticulocalbin 3, EF-hand calcium binding domain | NM_020650 | 28626509 | RCN3 |
| | (RCN3), mRNA | | | |
| 2039 | Homo sapiens cDNA FLJ38612 fis, clone HEART2006195, weakly | AK095931 | 21755290 | |
| | similar to Homo sapiens mRNA for myopodin | | | |
| 2042 | Homo sapiens cDNA FLJ38173 fis, clone FCBBF1000053, highly | AK095492 | 21754757 | |
| | similar to HYDROXYMETHYLGLUTARYL-COA SYNTHASE, | | | |
| | CYTOPLASMIC (EC 4.1.3.5) | | | |
| 2044 | Homo sapiens cDNA FLJ37260 fis, clone BRAMY2010441, highly | AK094579 | 21753666 | |
| | similar to PEANUT-LIKE PROTEIN 2 | | | |
| 2055 | Homo sapiens cortactin, mRNA (cDNA clone MGC: 33091 | BC033889 | 21707901 | |
| | IMAGE: 4824206), complete cds | | | |
| 2095 | Homo sapiens hypothetical protein FLJ20152 (FLJ20152), mRNA | NM_019000 | 21361616 | FLJ20152 |
| 2100 | Homo sapiens glycogen synthase kinase 3 beta (GSK3B), mRNA | NM_002093 | 21361339 | GSK3B |
| 2122 | Homo sapiens peptidylglycine alpha-amidating monooxygenase (PAM), | NM_138822 | 21070981 | PAM |
| | transcript variant 4, mRNA | | | |
| 2132 | Homo sapiens mRNA for KIAA1412 protein, partial cds | AB037833 | 20521903 | |
| 2135 | Homo sapiens clusterin (complement lysis inhibitor, SP-40, 40, sulfated | BC029972 | 20455818 | CLU |
| | glycoprotein 2, testosterone-repressed prostate message 2, | | | |
| | apolipoprotein J), mRNA (cDNA clone IMAGE: 4939961) | | | |
| 2152 | Homo sapiens angiopoietin-like 7 (ANGPTL7), mRNA | NM_021146 | 20127595 | ANGPTL7 |
| 2156 | Homo sapiens transmembrane protein 49 (TMEM49), mRNA | NM_030938 | 20070348 | TMEM49 |
| 2228 | Homo sapiens wingless-type MMTV integration site family, member 7B | NM_058238 | 17505192 | WNT7B |
| | (WNT7B), mRNA | | | |
| 2252 | Homo sapiens mRNA for KIAA0273 gene, partial cds | D87463 | 1665810 | |
| 2255 | Homo sapiens immediate early response 3 (IER3), transcript variant | NM_052815 | 16554596 | IER3 |
| | long, mRNA | | | |
| 2260 | Homo sapiens cDNA FLJ32730 fis, clone TESTI2001099, highly similar | AK057292 | 16552923 | |
| | to Homo sapiens homolog of the Aspergillus nidulans sudD gene | | | |
| | product mRNA | | | |
| 2264 | Homo sapiens cDNA FLJ30515 fis, clone BRAWH2000686, highly | AK055077 | 16549730 | |
| | similar to SERINE/THREONINE-PROTEIN KINASE SGK (EC 2.7.1.—) | | | |
| 2290 | Homo sapiens sparc/osteonectin, cwcv end kazal-like domains | NM_004598 | 15451924 | SPOCK |
| | proteoglycan (testican) (SPOCK), mRNA | | | |
| 2376 | Homo sapiens transmembrane protein 38A (TMEM38A), mRNA | NM_024074 | 13129059 | TMEM38A |
| 2377 | Homo sapiens endothelial differentiation, sphingolipid G-protein- | NM_001400 | 13027635 | EDG1 |
| | coupled receptor, 1 (EDG1), mRNA | | | |
| 2380 | cand_Human_FGA_30E10_ECM1_long_26Jun2003.fa | NM_004425 | 12707564 | ECM1 |
| | BaseSame: gi\|12707564\|ref\|NM_004425.2\|long variant of ECM1 Homo | | | |
| | sapiens extracellular matrix protein 1 (ECM1), transcript variant 1, | | | |
| | mRNA | | | |
| 2389 | Homo sapiens interferon induced transmembrane protein 3 (1-8U) | NM_021034 | 11995467 | IFITM3 |
| | (IFITM3), mRNA | | | |
| 2397 | Homo sapiens matrix metalloproteinase 2 (gelatinase A, 72 kDa | NM_004530 | 11342665 | MMP2 |
| | gelatinase, 72 kDa type IV collagenase) (MMP2), mRNA | | | |
| 2415 | Homo sapiens serpin peptidase inhibitor, clade E (nexin, plasminogen | NM_000602 | 10835158 | SERPINE1 |
| | activator inhibitor type 1), member 1 (SERPINE1), mRNA | | | |
| 2418 | Homo sapiens cDNA: FLJ23553 fis, clone LNG09301 | AK027206 | 10440278 | |
| 2419 | cr: gi\|10439471\|dbj\|AK026587.1\|Homo sapiens cDNA: FLJ22934 fis, | AK026587 | 10439471 | |
| | clone KAT07572, highly similar to AF100759 Homo sapiens | | | |
| | transmembrane 4 superfamily protein mRNA | | | |
| 2428 | Homo sapiens FXYD domain containing ion transport regulator 5 | NM_144779.1 | 47778937 | FXYD5 |
| | (FXYD5), transcript variant 1, mRNA | | | |

TABLE O

| | Description | Acc | Gi | Symbol |
|---|---|---|---|---|
| | Homo sapiens coiled-coil domain containing 80 (CCDC80), transcript variant 1, mRN | NM_199511 | 41152073 | CCDC80 |
| | | | | (SSG1) |
| | Homo sapiens synaptopodin 2 (SYNPO2), mRNA | NM_133477 | 55741765 | SYNPO2 |
| 1 | full-length cDNA clone CS0DH001YN15 of T cells (Jurkat cell line) of Homo | CR603770 | 50484577 | |
| | sapiens (human) | | | |
| 4 | Homo sapiens plectin 1, intermediate filament binding protein 500 kDa (PLEC1), | NM_201382 | 41322918 | PLEC1 |
| | transcript variant 8, mRNA | | | |
| 5 | Homo sapiens heterogeneous nuclear ribonucleoprotein A3 (HNRPA3), mRNA | NM_194247 | 34740328 | HNRPA3 |
| 9 | Homo sapiens actin, gamma 1 (ACTG1), mRNA | NM_001614 | 11038618 | ACTG1 |
| 10 | H. sapiens DNA sequence | Z22320 | 608801 | |
| 12 | Homo sapiens tubulin, alpha, ubiquitous (K-ALPHA-1), mRNA | NM_006082 | 57013275 | K-ALPHA-1 |
| 14 | H. sapiens mRNA for M-phase phosphoprotein, mpp4, 1523bp | X98264 | 1770455 | |
| 17 | cr: gi\|62897350\|dbj\|AK222896.1\|Homo sapiens mRNA for tumor necrosis factor | AK222896 | 62897350 | CD40 |
| | receptor superfamily, member 5 isoform 1 precursor variant, clone: HRC00913 | | | |
| 18 | Homo sapiens mRNA for notch1 preproprotein variant protein | AB209873 | 62089331 | |
| 19 | Homo sapiens lymphocyte-specific protein 1 (LSP1), transcript variant 3, mRNA | NM_001013254 | 61742790 | LSP1 |
| 20 | Homo sapiens qulescin Q6 (QSCN6), transcript variant 2, mRNA | NM_001004128 | 52493188 | QSCN6 |
| 24 | Homo sapiens single-stranded DNA binding protein 1 (SSBP1), mRNA | NM_003143 | 4507230 | SSBP1 |
| 26 | Homo sapiens aldolase A, fructose-bisphosphate (ALDOA), transcript variant 2, | NM_184041 | 34577109 | ALDOA |
| | mRNA | | | |
| 27 | Homo sapiens eukaryotic translation elongation factor 2 (EEF2), mRNA | NM_001961 | 25453476 | EEF2 |
| 28 | Human phospholemman chloride channel mRNA, complete cds | U72245 | 1916009 | |
| 29 | Homo sapiens myosin, light polypeptide 6, alkali, smooth muscle and non-muscle | NM_079423 | 17986259 | MYL6 |
| | (MYL6), transcript variant 2, mRNA | | | |
| 30 | Homo sapiens bromodomain containing 3 (BRD3), mRNA | NM_007371 | 12408642 | BRD3 |
| 31 | cand_Human_FGA_102E5_KIAA0537_26Jun2003.fa | NM_014840 | 7662169 | KIAA0537 |
| | BaseSame: gi\|7662169\|ref\|NM_014840.1\|Homo sapiens KIAA0537 gene product | | | |
| | (KIAA0537), mRNA | | | |
| 33 | cr: gi\|7022513\|dbj\|AK001326.1\|Homo sapiens cDNA FLJ10464 fis, clone | AK001326 | 7022513 | |
| | NT2RP1001546, weakly similar to PLATELET-ENDOTHELIAL TETRASPAN | | | |
| | ANTIGEN 3 | | | |
| 34 | Homo sapiens tubulin, beta 2 (TUBB2), mRNA | NM_001069 | 68299771 | TUBB2 |
| 35 | Homo sapiens mRNA; cDNA DKFZp566D244 (from clone DKFZp566D244) | AL122091 | 6102902 | |
| 37 | Human flg (fms-like gene) mRNA for putative protein tyrosine kinase (partial) | Y00665 | 558583 | partial |
| 38 | Homo sapiens major histocompatibility complex, class I, A (HLA-A), mRNA | NM_002116 | 52426770 | HLA-A |
| 39 | PREDICTED: Homo sapiens tangerin (DKFZp762C186), mRNA | XM_170658 | 51468744 | DKFZp762C186 |
| 40 | PREDICTED: Homo sapiens major histocompatibility complex, class I, H | XR_000297 | 51465695 | HLA-H |
| | (pseudogene) (HLA-H), misc RNA | | | |
| 41 | Homo sapiens tubulin, beta 3 (TUBB3), mRNA | NM_006086 | 50592995 | TUBB3 |
| 42 | full-length cDNA clone CS0DC005YH06 of Neuroblastoma Cot 25-normalized of | CR605423 | 50486230 | |
| | Homo sapiens (human) | | | |
| 43 | Homo sapiens muscleblind-like 2 (Drosophila) (MBNL2), transcript variant 1, | NM_144778 | 46411178 | MBNL2 |
| | mRNA | | | |
| 44 | Homo sapiens transcription factor 4 (TCF4), mRNA | NM_003199 | 4507398 | TCF4 |
| 45 | Homo sapiens filamin A, alpha (actin binding protein 280) (FLNA), mRNA | NM_001456 | 4503744 | FLNA |
| 46 | Homo sapiens TAR DNA binding protein (TARDBP), mRNA | NM_007375 | 42741653 | TARDBP |
| 47 | Homo sapiens serine/arginine repetitive matrix 1 (SRRM1), mRNA | NM_005839 | 42542378 | SRRM1 |
| 48 | Homo sapiens amyloid beta (A4) precursor protein (peptidase nexin-II, Alzheimer | NM_201414 | 41406056 | APP |
| | disease) (APP), transcript variant 3, mRNA | | | |
| 49 | Homo sapiens nucleophosmin (nucleolar phosphoprotein B23, numatrin) (NPM1), | NM_002520 | 40353732 | NPM1 |
| | mRNA | | | |
| 50 | Homo sapiens WNK lysine deficient protein kinase 1, mRNA (cDNA clone | BC013629 | 34783913 | |
| | IMAGE: 3445410), partial cds | | | |
| 51 | Homo sapiens chromosome 20 open reading frame 149 (C20orf149), mRNA | NM_024299 | 34147371 | C20orf149 |
| 52 | Homo sapiens dermokine (ZD52F10), mRNA | NM_033317 | 33504488 | ZD52F10 |
| 53 | Homo sapiens fracture callus 1 homolog (rat) (FXC1), mRNA | NM_012192 | 29837656 | FXC1 |
| 54 | Homo sapiens sterol regulatory element binding transcription factor 2 (SREBF2), | NM_004599 | 27477112 | SREBF2 |
| | mRNA | | | |
| 55 | H. sapiens telomeric DNA sequence, clone 22QTEL001, read 22QTELOO001.seq | Z96497 | 2181572 | |
| 56 | Homo sapiens platelet/endothelial cell adhesion molecule (CD31 antigen) | NM_000442 | 21314616 | PECAM1 |
| | (PECAM1), mRNA | | | |
| 59 | Homo sapiens tubulin, alpha 3 (TUBA3), mRNA | NM_006009 | 17986282 | TUBA3 |
| 61 | Homo sapiens von Willebrand factor (VWF), mRNA | NM_000552 | 9257255 | VWF |
| 62 | Homo sapiens hypothetical protein FLJ11078 (FLJ11078), mRNA | NM_018316 | 8922853 | FLJ11078 |
| 63 | Homo sapiens LUC7-like 2 (S. cerevisiae) (LUC7L2), mRNA | NM_016019 | 7706309 | LUC7L2 |
| 64 | Homo sapiens chromosome 11 open reading frame 10 (C11orf10), mRNA | NM_014206 | 7656933 | C11orf10 |
| 65 | Homo sapiens thymosin, beta 10 (TMSB10), mRNA | NM_021103 | 72255578 | TMSB10 |
| 67 | Homo sapiens vascular endothelial growth factor (VEGF), transcript variant 4, | NM_001025368 | 71051580 | VEGF |
| | mRNA | | | |
| 70 | Homo sapiens aspartyl aminopeptidase (DNPEP), mRNA | NM_012100 | 6912247 | DNPEP |
| 71 | Homo sapiens interleukin-1 receptor-associated kinase 1 (IRAK1), transcript | NM_001569 | 68800242 | IRAK1 |
| | variant 1, mRNA | | | |
| 73 | Homo sapiens complement component 4B (C4B), mRNA | NM_000592 | 67782350 | C4B |
| 74 | Homo sapiens lysyl oxidase-like 2 (LOXL2), mRNA | NM_002318 | 67782347 | LOXL2 |
| 75 | Homo sapiens ubiquitin C (UBC), mRNA | NM_021009 | 67191207 | UBC |
| 76 | cand_Human_FUN_DOP1.36_KIAA0538_26Jun2003.fa | AB011110 | 6635196 | |
| | BaseSame: gi\|6635196\|dbj\|AB011110.2\|Homo sapiens mRNA for KIAA0538 | | | |
| | protein, partial cds | | | |
| 77 | Homo sapiens KIAA0265 protein (KIAA0265), mRNA | NM_014997 | 66348151 | KIAA0265 |
| 78 | Homo sapiens tropomyosin 1 (alpha) (TPM1), transcript variant 1, mRNA | NM_001018005 | 63252897 | TPM1 |
| 79 | Homo sapiens far upstream element (FUSE) binding protein 1 (FUBP1), mRNA | NM_003902 | 62868228 | FUSE |
| 81 | Homo sapiens mRNA for dystrobrevin alpha isoform 2 variant protein | AB209102 | 62087783 | |
| 82 | Human nuclear factor I (NFI) mRNA, clone AT2, partial cds | U18760 | 619727 | NFI |
| 83 | Homo sapiens platelet-derived growth factor receptor, alpha polypeptide | NM_006206 | 61699224 | PDGFRA |
| | (PDGFRA), mRNA | | | |
| 84 | Homo sapiens tetratricopeptide repeat domain 7B (TTC7B), mRNA | NM_001010854 | 60685230 | TTC7B |
| 85 | Homo sapiens RAN, member RAS oncogene family (RAN), mRNA | NM_006325 | 6042206 | RAN |
| 86 | Homo sapiens mRNA; cDNA DKFZp686P12164 (from clone DKFZp686P12164) | CR936816 | 60219727 | |
| 87 | Homo sapiens CASK interacting protein 1 (CASKIN1), mRNA | NM_020764 | 59891449 | CASKIN1 |
| 88 | Homo sapiens zyxin (ZYX), transcript variant 2, mRNA | NM_001010972 | 58530844 | ZYX |
| 89 | Homo sapiens HMG2 like (LOC127540), transcript variant 2, mRNA | NM_001008728 | 57232747 | LOC127540 |
| 90 | Homo sapiens basic transcription factor 3-like 4 (BTF3L4), mRNA | NM_152265 | 56847619 | BTF3L4 |
| 91 | Homo sapiens 24-dehydrocholesterol reductase (DHCR24), mRNA | NM_014762 | 56790943 | DHCR24 |
| 92 | Homo sapiens ferritin, heavy polypeptide 1 (FTH1), mRNA | NM_002032 | 56682958 | FTH1 |
| 93 | Homo sapiens epoxide hydrolase 2, cytoplasmic (EPHX2), mRNA | NM_001979 | 56682950 | EPHX2 |
| 94 | Homo sapiens PAK1 interacting protein 1 (PAK1IP1), mRNA | NM_017906 | 55769545 | PAK1IP1 |
| 95 | Homo sapiens RUN and SH3 domain containing 2 (RUSC2), mRNA | NM_014806 | 55741718 | RUSC2 |
| 96 | Homo sapiens transforming, acidic coiled-coil containing protein 3 (TACC3), | NM_006342 | 5454101 | TACC3 |
| | mRNA | | | |
| 97 | Human platelet-endothelial tetraspan antigen 3 mRNA, complete cds | U14650 | 541612 | |
| 100 | Homo sapiens PTX1 protein (PTX1), mRNA | NM_016570 | 50959175 | PTX1 |
| 101 | Homo sapiens olfactomedin-like 3 (OLFML3), mRNA | NM_020190 | 50593011 | OLFML3 |
| 102 | Homo sapiens transmembrane BAX inhibitor motif containing 1 (TMBIM1), mRNA | NM_022152 | 50593007 | TMBIM1 |
| 103 | full-length cDNA clone CS0DJ002YN19 of T cells (Jurkat cell line) Cot 10- | CR610067 | 50490874 | |
| | normalized of Homo sapiens (human) | | | |
| 104 | full-length cDNA clone CL0BA012ZA08 of Placenta of Homo sapiens (human) | CR609237 | 50490044 | |
| 105 | full-length cDNA clone CS0DB001YD10 of Neuroblastoma Cot 10-normalized of | CR602380 | 50483187 | |
| | Homo sapiens (human) | | | |
| 106 | Homo sapiens serpin peptidase inhibitor, clade A (alpha-1 antiproteinase, | NM_001002236 | 50363218 | SERPINA1 |
| | antitrypsin), member 1 (SERPINA1), transcript variant 2, mRNA | | | |
| 107 | Homo sapiens lactate dehydrogenase A (LDHA), mRNA | NM_005566 | 5031856 | LDHA |
| 108 | Homo sapiens heat shock 27 kDa protein 1 (HSPB1), mRNA | NM_001540 | 4996892 | HSPB1 |
| 109 | Homo sapiens ATPase, H+ transporting, lysosomal 42 kDa, V1 subunit C isoform 2 | NM_144583 | 47717097 | ATP6V1C2 |
| | (ATP6V1C2), mRNA | | | |
| 110 | Homo sapiens chloride channel, calcium activated, family member 3 (CLCA3), | NM_004921 | 4757997 | CLCA3 |
| | mRNA | | | |
| 111 | Homo sapiens DNA-binding protein (APRF) mRNA, complete cds | L29277 | 475788 | APRF |
| 112 | Homo sapiens BCL2/adenovirus E1B 19 kDa interacting protein 2 (BNIP2), mRNA | NM_004330 | 4757855 | BNIP2 |
| 113 | Homo sapiens chromosome 11 open reading frame 31 (C11orf31), mRNA | NM_170746 | 46370090 | C11orf31 |
| 114 | Homo sapiens reticulon 1 (RTN1), transcript variant 2, mRNA | NM_206857 | 45827777 | RTN1 |
| 115 | Homo sapiens immature colon carcinoma transcript 1 (ICT1), mRNA | NM_001545 | 4557656 | ICT1 |
| 116 | Homo sapiens tumor protein, translationally-controlled 1 (TPT1), mRNA | NM_003295 | 4507668 | TPT1 |
| 117 | Homo sapiens proteolipid protein 2 (colonic epithelium-enriched) (PLP2), mRNA | NM_002668 | 4505892 | PLP2 |
| 118 | Homo sapiens GDP dissociation inhibitor 1 (GDI1), mRNA | NM_001493 | 4503970 | GDI1 |
| 119 | Homo sapiens cyclin-dependent kinase 8 (CDK8), mRNA | NM_001260 | 4502744 | CDK8 |
| 120 | Homo sapiens betaine-homocysteine methyltransferase (BHMT), mRNA | NM_001713 | 4502406 | BHMT |
| 121 | Homo sapiens KIAA0182 protein (KIAA0182), mRNA | NM_014615 | 44955925 | KIAA0182 |
| 122 | Homo sapiens tripartite motif-containing 23 (TRIM23), transcript variant alpha, | NM_001656 | 44955890 | TRIM23 |
| | mRNA | | | |
| 124 | Homo sapiens dystrobrevin, alpha (DTNA), transcript variant 4, mRNA | NM_032978 | 42718000 | DTNA |
| 126 | Homo sapiens EGF-like-domain, multiple 7 (EGFL7), transcript variant 2, mRNA | NM_201446 | 41393582 | EGFL7 |
| 127 | Homo sapiens leucine-rich repeats and immunoglobulin-like domains 3 (LRIG3), | NM_153377 | 40255156 | LRIG3 |
| | mRNA | | | |
| 128 | Homo sapiens inhibitor of growth family, member 4 (ING4), transcript variant 1, | NM_016162 | 38201669 | ING4 |
| | mRNA | | | |
| 129 | Homo sapiens eukaryotic translation initiation factor 4 gamma, 1 (EIF4G1), | NM_198242 | 38201624 | EIF4G1 |
| | transcript variant 4, mRNA | | | |
| 130 | Homo sapiens SAM domain and HD domain 1 (SAMHD1), mRNA | NM_015474 | 38016913 | SAMHD1 |
| 131 | Homo sapiens dystonin (DST), transcript variant 1eA, mRNA | NM_015548 | 34577048 | DST |
| 132 | Homo sapiens CCR4-NOT transcription complex, subunit 7 (CNOT7), transcript | NM_013354 | 34335233 | CNOT7 |
| | variant 1, mRNA | | | |
| 134 | Homo sapiens cyclin B1 (CCNB1), mRNA | NM_031966 | 34304372 | CCNB1 |
| 136 | Homo sapiens KIAA1533 (KIAA1533), mRNA | NM_020895 | 34147343 | KIAA1533 |
| 137 | Homo sapiens patatin-like phospholipase domain containing 2 (PNPLA2), mRNA | NM_020376 | 34147340 | PNPLA2 |
| 138 | Homo sapiens hypothetical protein ET, mRNA (cDNA clone MGC: 3827 | BC002753 | 33877122 | |
| | IMAGE: 3629029), complete cds | | | |
| 139 | Homo sapiens YEATS domain containing 2 (YEATS2), mRNA | NM_018023 | 33620754 | YEATS2 |
| 140 | Homo sapiens polymerase (RNA) III (DNA directed) polypeptide F, 39 kDa | NM_006466 | 33598951 | RNA |
| | (POLR3F), mRNA | | | |
| 141 | Homo sapiens neuronatin (NNAT), transcript variant 1, mRNA | NM_005386 | 32307134 | NNAT |
| 142 | Homo sapiens high-mobility group box 1 (HMGB1), mRNA | NM_002128 | 31982879 | HMGB1 |
| 143 | Homo sapiens Sjogren syndrome antigen B (autoantigen La) (SSB), mRNA | NM_003142 | 31543654 | SSB |
| 144 | Homo sapiens CXXC finger 5 (CXXC5), mRNA | NM_016463 | 31542945 | CXXC5 |
| 145 | Homo sapiens hypothetical protein FLJ20244 (FLJ20244), mRNA | NM_017722 | 31542702 | FLJ20244 |
| 146 | cr: gi\|3152702\|gb\|AF065389.1\|AF065389 Homo sapiens tetraspan NET-4 mRNA, | AF065389 | 3152702 | |
| | complete cds | | | |
| 147 | Homo sapiens splicing factor Sip1 mRNA, complete cds | AF030234 | 2822459 | |
| 148 | Homo sapiens lamin A/C (LMNA), transcript variant 2, mRNA | NM_005572 | 27436944 | LMNA |
| 150 | Homo sapiens ataxin 2-like (ATXN2L), transcript variant D, mRNA | NM_148415 | 27262650 | ATXN2L |
| 151 | Homo sapiens dispatched homolog 1 (Drosophila) (DISP1), mRNA | NM_032890 | 25952133 | DISP1 |
| 152 | Homo sapiens phosphoglycerate dehydrogenase (PHGDH), mRNA | NM_006623 | 23308576 | PHGDH |
| 153 | Homo sapiens actin related protein 2/3 complex, subunit 5, 16 kDa (ARPC5), | NM_005717 | 23238212 | ARPC5 |
| | mRNA | | | |
| 154 | Homo sapiens glutathione S-transferase M1 (GSTM1), transcript variant 1, mRNA | NM_000561 | 23065543 | GSTM1 |
| 155 | Homo sapiens hypothetical protein MGC35043 (MGC35043), mRNA | NM_152770 | 22749508 | MGC35043 |
| 156 | Homo sapiens cDNA FLJ36604 fis, clone TRACH2015243, highly similar to | AK093923 | 21752881 | |
| | KENDRIN | | | |
| 157 | Homo sapiens chromosome 1 open reading frame 91 (C1orf91), mRNA | NM_019118 | 21361813 | C1orf91 |
| 158 | Homo sapiens nucleolar protein 11 (NOL11), mRNA | NM_015462 | 21361467 | NOL11 |
| 159 | Homo sapiens heat shock 70 kDa protein 5 (glucose-regulated protein, 78 kDa) | NM_005347 | 21361242 | HSPA5 |
| | (HSPA5), mRNA | | | |
| 160 | Homo sapiens plasma glutamate carboxypeptidase (PGCP), mRNA | NM_016134 | 21314657 | PGCP |
| 161 | cand_Human_STR_81G10_FL_26Jun2003.fa | BC030705 | 21265144 | |
| | BaseMod: gi\|21265144\|gb\|BC030705.1\|Based on Homo sapiens clone | | | |
| | MGC: 27076 IMAGE: 4824344, mRNA, complete cds | | | |
| 162 | Homo sapiens chromobox homolog 3 (HP1 gamma homolog, Drosophila) (CBX3), | NM_016587 | 20544150 | CBX3 |
| | transcript variant 2, mRNA | | | |
| 163 | Homo sapiens DEAH (Asp-Glu-Ala-His) box polypeptide 30 (DHX30), transcript | NM_138615 | 20336293 | DHX30 |
| | variant 1, mRNA | | | |
| 164 | Homo sapiens phosphomevalonate kinase (PMVK), mRNA | NM_006556 | 20127505 | PMVK |
| 165 | Homo sapiens putative translation initiation factor (SUI1), mRNA | NM_005801 | 20070210 | SUI1 |
| 166 | Homo sapiens dysferlin, limb girdle muscular dystrophy 2B (autosomal recessive) | NM_003494 | 19743938 | DYSF |
| | (DYSF), mRNA | | | |
| 167 | Homo sapiens chromosome 6 open reading frame 111 (C6orf111), mRNA | NM_032870 | 18699723 | C6orf111 |
| 168 | Homo sapiens shroom (SHRM), mRNA | NM_020859 | 18699721 | SHRM |
| 169 | Homo sapiens cDNA FLJ32140 fis, clone PLACE5000058, weakly similar to | AK056702 | 16552180 | |
| | PUTATIVE IMPORTIN BETA-4 SUBUNIT | | | |
| 170 | Homo sapiens cDNA FLJ31748 fis, clone NT2RI2007384, highly similar to Homo | AK056310 | 16551675 | |
| | sapiens mixed lineage kinase mRNA | | | |
| 171 | Homo sapiens cingulin (CGN), mRNA | NM_020770 | 16262451 | CGN |
| 174 | Homo sapiens cytochrome P450, family 3, subfamily A, polypeptide 5 (CYP3A5), | NM_000777 | 15147331 | CYP3A5 |
| | mRNA | | | |
| 175 | Homo sapiens spermatogenesis associated 16 (SPATA16), mRNA | NM_031955 | 15100157 | SPATA16 |
| 176 | Homo sapiens emopamil binding protein-like (EBPL), mRNA | NM_032565 | 14211872 | EBPL |
| 177 | Homo sapiens protocadherin gamma subfamily A, 11 (PCDHGA11), transcript | NM_018914 | 14196449 | PCDHGA11 |
| | variant 1, mRNA | | | |
| 179 | Homo sapiens zinc finger, CCHC domain containing 9 (ZCCHC9), mRNA | NM_032280 | 14150026 | ZCCHC9 |
| 180 | Homo sapiens heterogeneous nuclear ribonucleoprotein F (HNRPF), mRNA | NM_004966 | 14141150 | HNRPF |
| 181 | Homo sapiens cDNA FLJ14791 fis, clone NT2RP4001064, weakly similar to | AK027697 | 14042570 | |
| | SYNAPTONEMAL COMPLEX PROTEIN SC65 | | | |
| 182 | Homo sapiens tRNA splicing endonuclease 34 homolog (SEN34, S. cerevisiae) | NM_024075 | 13129061 | TSEN34 |
| | (TSEN34), mRNA | | | |
| 183 | Homo sapiens serine racemase (SRR), mRNA | NM_021947 | 11345491 | SRR |
| 184 | Homo sapiens interferon induced transmembrane protein 2 (1-8D) (IFITM2), | NM_006435 | 10835237 | IFITM2 |
| | mRNA | | | |
| 185 | Homo sapiens neural cell adhesion molecule 1 (NCAM1), transcript variant 1, | NM_000615 | 10834989 | NCAM1 |
| | mRNA | | | |
| 186 | Homo sapiens eukaryotic translation initiation factor 4 gamma, 3 (EIF4G3), mRNA | NM_003760 | 10092600 | EIF4G3 |
| 187 | Homo sapiens synovial sarcoma translocation gene an chromosome 18-like 2 | NM_016305 | 10047103 | SS18L2 |
| | (SS18L2), mRNA | | | |
| 188 | Homo sapiens reticulon 4a mRNA, complete cds | AF148537 | 10039550 | |
| 189 | Homo sapiens BAI1-associated protein 2 (BAIAP2), transcript variant 2, mRNA | NM_017451 | 9257198 | BAIAP2 |
| 190 | Homo sapiens BAI1-associated protein 2 (BAIAP2), transcript variant 1, mRNA | NM_017450 | 9257196 | BAIAP2 |
| 191 | Homo sapiens heme oxygenase (decycling) 2 (HMOX2), mRNA | NM_002134 | 8051607 | HMOX2 |
| 192 | Homo sapiens LSM7 homolog, U6 small nuclear RNA associated (S. cerevisiae) | NM_016199 | 7706422 | LSM7 |
| | (LSM7), mRNA | | | |
| 193 | Homo sapiens sorting nexing 24 (SNX24), mRNA | NM_014035 | 7662654 | SNX24 |
| 194 | Homo sapiens basic leucine zipper and W2 domains 2 (BZW2), mRNA | NM_014038 | 7661743 | BZW2 |
| 195 | Homo sapiens nucleolar complex associated 2 homolog (S. cerevisiae) (NOC2L), | NM_015658 | 7661605 | NOC2L |
| | mRNA | | | |
| 196 | Homo sapiens brain protein 44 (BRP44), mRNA | NM_015415 | 7661601 | BRP44 |
| 198 | Homo sapiens aspartoacylase (Canavan disease) (ASPA), mRNA | NM_000049 | 73622272 | ASPA |
| 199 | Homo sapiens chloride intracellular channel 4 (CLIC4), mRNA | NM_013943 | 7330334 | CLIC4 |
| 200 | Homo sapiens sex comb an midleg homolog 1 (Drosophila) (SCMH1), transcript | NM_001031694 | 72534679 | SCMH1 |
| | variant 1, mRNA | | | |
| 203 | Homo sapiens activating transcription factor 3 (ATF3), transcript variant 1, mRNA | NM_001674 | 71902534 | ATF3 |
| 204 | Homo sapiens mRNA for KIAA0516 protein, partial cds | AB011088 | 71891761 | |
| 205 | Homo sapiens mRNA for KIAA0774 protein, partial cds | AB018317 | 71891682 | |
| 206 | Homo sapiens adenine phosphoribosyltransferase (APRT), transcript variant 1, | NM_000485 | 71773149 | APRT |
| | mRNA | | | |
| 211 | Homo sapiens breast carcinoma-associated antigen isoform I mRNA, partial cds | AF227899 | 7110492 | |
| 212 | Homo sapiens vascular endothelial growth factor (VEGF), transcript variant 6, | NM_001025370 | 71051584 | VEGF |
| | mRNA | | | |
| 213 | Homo sapiens N-acylsphingosine amidohydrolase 3-like (ASAH3L), mRNA | NM_001010887 | 71043497 | ASAH3L |
| 214 | Homo sapiens peptidase inhibitor 16 (PI16), mRNA | NM_153370 | 70780383 | PI16 |
| 216 | Homo sapiens tumor protein D52 (TPD52), transcript variant 2, mRNA | NM_001025253 | 70608173 | TPD52 |
| 217 | Homo sapiens hypothetical protein FLJ20530 (FLJ20530), mRNA | NM_017864 | 70608108 | FLJ20530 |
| 218 | Homo sapiens cDNA FLJ10319 fis, clone NT2RM2000490, weakly similar to | AK001181 | 7022274 | |
| | SYNAPTOTAGMIN | | | |
| 219 | Homo sapiens LSM5 homolog, U6 small nuclear RNA associated (S. cerevisiae) | NM_012322 | 6912487 | LSM5 |
| | (LSM5), mRNA | | | |
| 220 | Homo sapiens general transcription factor IIIC, polypeptide 3, 102 kDa (GTF3C3), | NM_012086 | 6912397 | GTF3C3 |
| | mRNA | | | |
| 221 | Homo sapiens brain expressed, X-linked 1 (BEX1), mRNA | NM_018476 | 68533248 | BEX1 |
| 222 | Homo sapiens heat shock transcription factor 2 (HSF2), mRNA | NM_004506 | 6806888 | HSF2 |
| 223 | Homo sapiens lysyl oxidase-like 1 (LOXL1), mRNA | NM_005576 | 67782345 | LOXL1 |
| 224 | Homo sapiens farnesyl-diphosphate farnesyltransferase 1 (FDFT1), mRNA | NM_004462 | 67089146 | FDFT1 |
| 225 | Homo sapiens alpha-2-macroglobulin (A2M), mRNA | NM_000014 | 66932940 | A2M |
| 226 | Homo sapiens tensin 1 (TNS1), mRNA | NM_022648 | 66529406 | TNS1 |
| 227 | Homo sapiens non-metastatic cells 2, protein (NM23B) expressed in (NME2), | NM_001018139 | 66392226 | NM23B |
| | transcript variant 5, mRNA | | | |
| 228 | Homo sapiens hemoglobin, zeta (HBZ), mRNA | NM_005332 | 6633805 | HBZ |
| 229 | Homo sapiens coronin, actin binding protein, 1B (CORO1B), transcript variant 2, | NM_001018070 | 65787363 | CORO1B |
| | mRNA | | | |
| 230 | Homo sapiens signal sequence receptor, beta (translocon-associated protein beta) | NM_003145 | 6552341 | SSR2 |
| | (SSR2), mRNA | | | |
| 231 | Homo sapiens glutathione S-transferase pi (GSTP1), mRNA | NM_000852 | 6552334 | GSTP1 |
| 232 | Homo sapiens RAN binding protein 1 (RANBP1), mRNA | NM_002882 | 6382077 | RANBP1 |
| 233 | Homo sapiens capping protein (actin filament), gelsolin-like (CAPG), mRNA | NM_001747 | 63252912 | CAPG |
| 234 | Homo sapiens procollagen-proline, 2-oxoglutarate 4-dioxygenase (proline 4- | NM_001017973 | 63252890 | P4HA2 |
| | hydroxylase), alpha polypeptide II (P4HA2), transcript variant 2, mRNA | | | |
| 235 | Homo sapiens modulator of estrogen induced transcription (FLJ13213), transcript | NM_024755 | 63176610 | FLJ13213 |
| | variant 1, mRNA | | | |
| 236 | Homo sapiens seven in absentia homolog 1 (Drosophila) (SIAH1), transcript | NM_003031 | 63148617 | SIAH1 |
| | variant 1, mRNA | | | |
| 237 | Homo sapiens DNA segment, Chr 15, Wayne State University 75, expressed | NM_015704 | 63055039 | D15Wsu75e |
| | (D15Wsu75e), mRNA | | | |
| 238 | Homo sapiens sirtuin (silent mating type information regulation 2 homolog) 3 (S. cerevisiae) | NM_001017524 | 63054861 | SIRT3 |
| | (SIRT3), transcript variant 2, mRNA | | | |
| 239 | Homo sapiens FLYWCH-type zinc finger 1 (FLYWCH1), transcript variant 1, | NM_032296 | 62953133 | FLYWCH1 |
| | mRNA | | | |
| 240 | Homo sapiens mRNA for heterogeneous nuclear ribonucleoprotein A1 isoform a | AK222845 | 62897248 | |
| | variant, clone: HEP09073 | | | |
| 241 | Homo sapiens sterol-C4-methyl oxidase-like (SC4MOL), transcript variant 1, | NM_006745 | 62865626 | SC4MOL |
| | mRNA | | | |
| 242 | Homo sapiens heparan sulfate proteoglycan 2 (periecan) (HSPG2), mRNA | NM_005529 | 62859978 | HSPG2 |
| 243 | Homo sapiens chromosome 6 open reading frame 82 (C6orf82), transcript variant | NM_015921 | 62526020 | C6orf82 |
| | 2, mRNA | | | |
| 244 | Homo sapiens polymerase (RNA) II (DNA directed) polypeptide K, 7.0 kDa | NM_005034 | 62422569 | RNA |
| | (POLR2K), mRNA | | | |
| 245 | Homo sapiens Tax1 (human T-cell leukemia virus type I) binding protein 3 | NM_014604 | 62339397 | TAX1BP3 |
| | (TAX1BP3), mRNA | | | |
| 246 | Homo sapiens DNA (cytosine-5-)-methyltransferase 1, mRNA (cDNA clone | BC092517 | 62204779 | DNMT1 |
| | MGC: 104992 IMAGE: 3077831), complete cds | | | |
| 247 | Homo sapiens mRNA for protein tyrosine phosphatase, receptor type, A isoform 2 | AB209702 | 62088983 | |
| | precursor variant protein | | | |
| 248 | Homo sapiens mRNA for protein tyrosine phosphatase, receptor type, sigma | AB209333 | 62088245 | |
| | isoform 3 precursor variant protein | | | |
| 249 | Homo sapiens mRNA for phosphoribosylglycinamide formyltransferase, | AB208785 | 62087149 | |
| | phosphoribosylglycinamide synthetase, phosphoribosylaminoimidazole synthetase | | | |
| | isoform 1 variant protein | | | |
| 250 | Homo sapiens lumican (LUM), mRNA | NM_002345 | 61742794 | LUM |
| 251 | Synthetic construct Homo sapiens clone FLH007279.01X eukaryotic translation | AY888456 | 61357519 | EIF3S2 |
| | initiation factor 3 subunit 2 beta (EIF3S2) mRNA, complete cds | | | |
| 252 | cr. gi\|60653756\|gb\|AY892655.1\|Synthetic construct Homo sapiens clone | AY892655 | 60653756 | ITGB5 |
| | FLH015900.01L integrin beta 5 (ITGB5) mRNA, partial cds | | | |
| 253 | Homo sapiens cross-immune reaction antigen PCIA1 (PCIA1), mRNA | NM_024050 | 60279701 | PCIA1 |
| 254 | Homo sapiens lectin, galactoside-binding, soluble, 4 (galectin 4) (LGALS4), mRNA | NM_006149 | 6006017 | LGALS4 |
| 255 | Homo sapiens midkine (neurite growth-promoting factor 2) (MDK), transcript | NM_001012334 | 59894791 | MDK |
| | variant 1, mRNA | | | |
| 256 | Homo sapiens ring finger protein CKBBP1 mRNA, complete cds | AF164679 | 5917673 | CKBBP1 |
| 257 | Homo sapiens septin 7 (SEPT7), transcript variant 1, mRNA | NM_001788 | 58535458 | SEPT7 |
| 258 | Homo sapiens forty-two-three domain containing 1 (FYTTD1), transcript variant 1, | NM_032288 | 58374126 | FYTTD1 |
| | mRNA | | | |
| 259 | Homo sapiens septin 5 (SEPT5), transcript variant 1, mRNA | NM_002688 | 58331275 | SEPT5 |
| 260 | Homo sapiens mRNA for KIAA0700 protein, partial cds | AB014600 | 58257659 | |
| 261 | Homo sapiens nucleoporin 205 kDa (NUP205), mRNA | NM_015135 | 57634533 | NUP205 |
| 263 | Homo sapiens synaptophysin (SYP), mRNA | NM_003179 | 56699493 | SYP |
| 264 | Homo sapiens troponin I, skeletal, slow (TNNI1), mRNA | NM_003281 | 56682968 | TNNI1 |
| 265 | Homo sapiens ferritin, light polypeptide (FTL), mRNA | NM_000146 | 56682960 | FTL |
| 266 | Homo sapiens transmembrane emp24 protein transport domain containing 3 | NM_007364 | 56676386 | TMED3 |
| | (TMED3), mRNA | | | |
| 267 | Homo sapiens poly(A) binding protein, cytoplasmic 1 (PABPC1), mRNA | NM_002568 | 56676313 | PABPC1 |
| 268 | Homo sapiens cingulin-like 1 (CGNL1), mRNA | NM_032866 | 56550102 | CGNL1 |
| 269 | Homo sapiens SMAD specific E3 ubiquitin protein ligase 2 (SMURF2), mRNA | NM_022739 | 56550041 | SMURF2 |
| 270 | Homo sapiens IGF-II mRNA-binding protein 1 (IMP-1), mRNA | NM_006546 | 56237026 | IMP-1 |
| 271 | Homo sapiens nuclear casein kinase and cyclin-dependent kinase substrate 1 | NM_022731 | 56118309 | NUCKS1 |
| | (NUCKS1), mRNA | | | |
| 272 | Homo sapiens secreted frizzled-related protein 1 (SFRP1), mRNA | NM_003012 | 56117837 | SFRP1 |
| 273 | Homo sapiens gem (nuclear organelle) associated protein 7 (GEMIN7), transcript | NM_024707 | 55953070 | GEMIN7 |
| | variant 1, mRNA | | | |
| 275 | Homo sapiens retinol binding protein 4, plasma (RBP4), mRNA | NM_006744 | 55743121 | RBP4 |
| 276 | Homo sapiens v-erb-b2 erythroblastic leukemia viral oncogene homolog 2, | NM_001005862 | 54792097 | ERBB2 |
| | neuro/glioblastoma derived oncogene homolog (avian) (ERBB2), transcript variant | | | |
| | 2, mRNA | | | |
| 277 | Homo sapiens ATM/ATR-Substrate Chk2-Interacting Zn2+-finger protein (ASCIZ), | NM_015251 | 54792091 | ASCIZ |
| | mRNA | | | |
| 278 | Homo sapiens prefoldin 4 (PFDN4), mRNA | NM_002623 | 54792079 | PFDN4 |
| 279 | Homo sapiens mucolipin 2 (MCOLN2), mRNA | NM_153259 | 54792072 | MCOLN2 |
| 280 | Homo sapiens SMT3 suppressor of mif two 3 homolog 2 (yeast) (SUMO2), | NM_006937 | 54792068 | SUMO2 |
| | transcript variant 1, mRNA | | | |
| 281 | Homo sapiens ras homolog gene family, member N mRNA, complete cds | BT019394 | 54695657 | |
| 282 | Homo sapiens acidic (leucine-rich) nuclear phosphoprotein 32 family, member B | NM_006401 | 5454087 | ANP32B |
| | (ANP32B), mRNA | | | |
| 283 | Homo sapiens Rab acceptor 1 (prenylated) (RABAC1), mRNA | NM_006423 | 5453959 | CAPZA1 |
| 284 | Homo sapiens capping protein (actin filament) muscle Z-line, alpha 1 (CAPZA1), | NM_006135 | 5453596 | CAPZA1 |
| | mRNA | | | |
| 286 | Homo sapiens Cas-Br-M (murine) ecotropic retroviral transforming sequence b | NM_170662 | 54112419 | CBLB |
| | (CBLB), mRNA | | | |
| 287 | Homo sapiens myelin gene expression factor 2 mRNA, complete cds | AF106685 | 5410335 | |
| 288 | Homo sapiens transitional endoplasmic reticulum ATPase mRNA, complete cds | AF100752 | 5410289 | |
| 289 | Homo sapiens tissue Inhibitor of metalloproteinase 2 (TIMP2), mRNA | NM_003255 | 52632388 | TIMP2 |
| 290 | Homo sapiens monocyte to macrophage differentiation-associated (MMD), mRNA | NM_012329 | 52630444 | MMD |
| 291 | Homo sapiens major histocompatibility complex, class I, C (HLA-C), mRNA | NM_002117 | 52630341 | HLA-C |
| 292 | Homo sapiens differential display and activated by p53 (DDA3), transcript variant | NM_032636 | 52630332 | DDA3 |
| | 1, mRNA | | | |
| 293 | Homo sapiens v-raf-1 murine leukemia viral oncogene homolog 1 (RAF1), mRNA | NM_002880 | 52486392 | RAF1 |
| 294 | Homo sapiens Cas-Br-M (murine) ecotropic retroviral transforming sequence | NM_005188 | 52426744 | CBL |
| | (CBL), mRNA | | | |
| 295 | Homo sapiens S100 calcium binding protein A6 (calcyclin) (S100A6), mRNA | NM_014624 | 52352807 | S100A6 |
| 296 | Homo sapiens fibulin 2 (FBLN2), transcript variant 2, mRNA | NM_001998 | 51873054 | FBLN2 |
| 297 | Homo sapiens RAN binding protein 5 (RANBP5), mRNA | NM_002271 | 51873051 | RANBP5 |
| 298 | Homo sapiens milk fat globule-EGF factor 8 protein (MFGE8), mRNA | NM_005928 | 5174556 | MFGE8 |
| 300 | PREDICTED: Homo sapiens similar to PI-3-kinase-related kinase SMG-1 isoform | XM_370939 | 51472863 | LOC388221 |
| | 1; lambda/iota protein kinase C-Interacting protein; phosphatidylinositol 3-kinase- | | | |
| | related protein kinase (LOC388221), mRNA | | | |
| 302 | PREDICTED: Homo sapiens RNA binding motif protein 20 (RBM20), mRNA | XM_291671 | 51467827 | RBM20 |
| 304 | PREDICTED: Homo sapiens similar to basic leucine zipper and W2 domains 1 | XM_045290 | 51464099 | LOC151579 |
| | (LOC151579), mRNA | | | |
| 305 | PREDICTED: Homo sapiens EphA6 (EPHA6), mRNA | XM_114973 | 51464074 | EPHA6 |
| 306 | PREDICTED: Homo sapiens hypothetical LOC388588 (LOC388588), mRNA | XM_373823 | 51458727 | LOC388588 |
| 310 | Homo sapiens checkpoint suppressor 1 (CHES1), mRNA | NM_005197 | 51093864 | CHES1 |
| 311 | Homo sapiens annexin A2 (ANXA2), transcript variant 3, mRNA | NM_004039 | 50845389 | ANXA2 |
| 312 | Homo sapiens male-enhanced antigen 1 (MEA1), mRNA | NM_014623 | 50726966 | MEA1 |
| 314 | Homo sapiens hydroxysteroid (17-beta) dehydrogenase 7 (HSD17B7), mRNA | NM_016371 | 50593017 | HSD17B7 |
| 315 | Homo sapiens ras homolog gene family, member A (RHOA), mRNA | NM_001664 | 50593005 | RHOA |
| 316 | full-length cDNA clone CS0DE009YF20 of Placenta of Homo sapiens (human) | CR626719 | 50507581 | |
| 317 | full-length cDNA clone CS0DF002YP14 of Fetal brain of Homo sapiens (human) | CR626356 | 50507163 | |
| 318 | full-length cDNA clone CS0DH006YI09 of T cells (Jurkat cell line) of Homo sapiens | CR622666 | 50503473 | |
| | (human) | | | |
| 319 | full-length cDNA clone CS0DC014YC18 of Neuroblastoma Cot 25-normalized of | CR618748 | 50499555 | |
| | Homo sapiens (human) | | | |
| 320 | full-length cDNA clone CS0DI081YB23 of Placenta Cot 25-normalized of Homo | CR617987 | 50498794 | |
| | sapiens (human) | | | |
| 321 | full-length cDNA clone CL0BA009ZC04 of Placenta of Homo sapiens (human) | CR614775 | 50495582 | |
| 322 | full-length cDNA clone CS0DG007YD04 of B cells (Ramos cell line) of Homo | CR612229 | 50493036 | |
| | sapiens (human) | | | |
| 323 | full-length cDNA clone CS0DI013YH09 of Placenta Cot 25-normalized of Homo | CR611898 | 50492705 | |
| | sapiens (human) | | | |
| 324 | full-length cDNA clone CS0DI020YO04 of Placenta Cot 25-normalized of Homo | CR608343 | 50489150 | |
| | sapiens (human) | | | |
| 325 | full-length cDNA clone CS0DD007YD16 of Neuroblastoma Cot 50-normalized of | CR608062 | 50488869 | |
| | Homo sapiens (human) | | | |
| 326 | full-length cDNA clone CS0DM007YI10 of Fetal liver of Homo sapiens (human) | CR607973 | 50488780 | |
| 327 | full-length cDNA clone CS0DK007YF11 of HeLa cells Cot 25-normalized of Homo | CR607189 | 50487996 | |
| | sapiens (human) | | | |
| 328 | full-length cDNA clone CS0DI010YH14 of Placenta Cot 25-normalized of Homo | CR606794 | 50487601 | |
| | sapiens (human) | | | |
| 329 | full-length cDNA clone CS0DI036YF07 of Placenta Cot 25-normalized of Homo | CR604907 | 50485714 | |
| | sapiens (human) | | | |
| 330 | full-length cDNA clone CL0BA006ZC10 of Placenta of Homo sapiens (human) | CR602328 | 50483135 | |
| 331 | full-length cDNA clone CS0DK008YI13 of HeLa cells Cot 25-normalized of Homo | CR598865 | 50479672 | |
| | sapiens (human) | | | |
| 332 | full-length cDNA clone CS0DF036YG02 of Fetal brain of Homo sapiens (human) | CR598750 | 50479557 | |
| 333 | full-length cDNA clone CS0DC001YH18 of Neuroblastoma Cot 25-normalized of | CR597404 | 50478211 | |
| | Homo sapiens (human) | | | |
| 334 | Homo sapiens Down syndrome critical region gene 1-like 1 (DSCR1L1), mRNA | NM_005822 | 5032234 | DSCR1L1 |
| 335 | Homo sapiens lysyl-tRNA synthetase (KARS), mRNA | NM_005548 | 5031814 | KARS |
| 336 | Homo sapiens heterogeneous nuclear ribonucleoprotein H1 (H) (HNRPH1), mRNA | NM_005520 | 5031752 | HNRPH1 |
| 337 | Homo sapiens high-mobility group nucleosomal binding domain 2 (HMGN2), | NM_005517 | 50233912 | HMGN2 |
| | mRNA | | | |
| 338 | Homo sapiens actin, beta (ACTB), mRNA | NM_001101 | 5016088 | ACTB |
| 339 | Homo sapiens runt-related transcription factor 1 (acute myeloid leukemia 1; aml1 | NM_001001890 | 49574545 | RUNX1 |
| | oncogene) (RUNX1), transcript variant 2, mRNA | | | |
| 340 | Homo sapiens sterol O-acyltransferase (acyl-Coenzyme A: cholesterol | NM_003101 | 49533616 | SOAT1 |
| | acyltransferase) 1 (SOAT1), transcript variant 688113, mRNA | | | |
| 341 | Homo sapiens DnaJ (Hsp40) homolog, subfamily A, member 1 (DNAJA1), mRNA | NM_001539 | 49472820 | Hsp40 |
| 342 | Homo sapiens brain protein I3 (BRI3), mRNA | NM_015379 | 48928020 | BRI3 |
| 343 | Homo sapiens neurofilament 3 (150 kDa medium) (NEF3), mRNA | NM_005382 | 4885512 | NEF3 |
| 344 | Homo sapiens guanine nucleotide binding protein (G protein), gamma 5 (GNG5), | NM_005274 | 4885286 | GNG5 |
| | mRNA | | | |
| 345 | Homo sapiens cysteine and glycine-rich protein 3 (cardiac LIM protein) (CSRP3), | NM_003476 | 48762935 | CSRP3 |
| | mRNA | | | |
| 346 | Homo sapiens collagen, type I, alpha 2 (COL1A2), mRNA | NM_000089 | 48762933 | COL1A2 |
| 347 | Homo sapiens v-ral simian leukemia viral oncogene homolog B (ras related; GTP | NM_002881 | 48762927 | RALB |
| | binding protein) (RALB), mRNA | | | |
| 348 | Homo sapiens PWP2 periodic tryptophan protein homolog (yeast) (PWP2H), | NM_005049 | 48762925 | PWP2H |
| | mRNA | | | |
| 349 | Homo sapiens SH3 domain binding glutamic acid-rich protein (SH3BGR), | NM_007341 | 48762710 | SH3BGR |
| | transcript variant 1, mRNA | | | |
| 350 | Homo sapiens eukaryotic translation elongation factor 1 alpha 1, mRNA (cDNA | BC071619 | 48735184 | |
| | clone MGC: 87736 IMAGE: 4448926), complete cds | | | |
| 351 | Homo sapiens secreted protein, acidic, cysteine-rich (osteonectin) (SPARC), | NM_003118 | 48675809 | SPARC |
| | mRNA | | | |
| 352 | Homo sapiens zinc finger protein 9 (a cellular retroviral nucleic acid binding | NM_003418 | 4827070 | ZNF9 |
| | protein) (ZNF9), mRNA | | | |
| 353 | Homo sapiens poly(A) polymerase alpha (PAPOLA), mRNA | NM_032632 | 47834324 | PAPOLA |
| 354 | Homo sapiens malignant fibrous histiocytoma amplified sequence 1 (MFHAS1), | NM_004225 | 4758703 | MFHAS1 |
| | mRNA | | | |
| 356 | Homo sapiens enhancer of rudimentary homolog (Drosophila) (ERH), mRNA | NM_004450 | 4758301 | ERH |
| 357 | Homo sapiens tryptophanyl-tRNA synthetase (WARS), transcript variant 2, mRNA | NM_173701 | 47419915 | WARS |
| 358 | Homo sapiens abhydrolase domain containing 6 (ABHD6), mRNA | NM_020676 | 47271494 | ABHD6 |
| 359 | Homo sapiens fibronectin 1 (FN1), transcript variant 1, mRNA | NM_212482 | 47132558 | FN1 |
| 360 | Homo sapiens actin, alpha 1, skeletal muscle (ACTA1), mRNA | NM_001100 | 47078293 | ACTA1 |
| 361 | Homo sapiens G protein pathway suppressor 1 (GPS1), transcript variant 2, mRNA | NM_004127 | 47078239 | GPS1 |
| 363 | Homo sapiens ubiquinol-cytochrome c reductase core protein I (UQCRC1), mRNA | NM_003365 | 46593006 | UQCRC1 |
| 364 | Homo sapiens astrotactin (ASTN), transcript variant 1, mRNA | NM_004319 | 46488922 | ASTN |
| 365 | Homo sapiens nasal embryonic LHRH factor (NELF), mRNA | NM_015537 | 46397395 | NELF |
| 366 | Homo sapiens similar to splicing factor, arginine/serine-rich 4 (FLJ11021), | NM_198261 | 46397365 | FLJ11021 |
| | transcript variant 2, mRNA | | | |
| 367 | Homo sapiens selenoprotein K (SELK), mRNA | NM_021237 | 46370091 | SELK |
| 368 | Homo sapiens PWWP domain containing 2 (PWWP2), mRNA | NM_138499 | 46358349 | PWWP2 |
| 369 | Homo sapiens KIAA0355 (KIAA0355), mRNA | NM_014686 | 46276867 | KIAA0355 |
| 370 | Homo sapiens parathymosin (PTMS), mRNA | NM_002824 | 46276862 | PTMS |
| 371 | Homo sapiens insulin-like growth factor binding protein 5 (IGFBP5), mRNA | NM_000599 | 46094066 | IGFBP5 |
| 372 | Homo sapiens peroxisome biogenesis factor 13 (PEX13), mRNA | NM_002618 | 46047483 | PEX13 |
| 373 | Homo sapiens SAM and SH3 domain containing 1 (SASH1), mRNA | NM_015278 | 45935384 | SASH1 |
| 374 | Homo sapiens UDP glucuronosyltransferase 1 family, polypeptide A6 (UGT1A6), | NM_001072 | 45827764 | UGT1A6 |
| | transcript variant 1, mRNA | | | |
| 375 | Homo sapiens ectonucleoside triphosphate diphosphohydrolase 2 (ENTPD2), | NM_203468 | 45827717 | ENTPD2 |
| | transcript variant 1, mRNA | | | |
| 376 | Homo sapiens quaking homolog, KH domain RNA binding (mouse) (QKI), | NM_006775 | 45827705 | QKI |
| | transcript variant 1, mRNA | | | |
| 377 | Homo sapiens mortality factor 4 like 1 (MORF4L1), transcript variant 1, mRNA | NM_006791 | 45643136 | MORF4L1 |
| 378 | Homo sapiens TNF receptor-associated factor 7 (TRAF7), transcript variant 1, | NM_032271 | 45594311 | TRAF7 |
| | mRNA | | | |
| 379 | Homo sapiens HLA-B associated transcript 1 (BAT1), transcript variant 1, mRNA | NM_004640 | 45580710 | BAT1 |
| 380 | Homo sapiens unc-93 homolog B1 (C. elegans) (UNC93B1), mRNA | NM_030930 | 45580708 | UNC93B1 |
| 381 | Homo sapiens myosin, heavy polypeptide 7, cardiac muscle, beta (MYH7), mRNA | NM_000257 | 4557772 | MYH7 |
| 382 | Homo sapiens v-kit Hardy-Zuckerman 4 feline sarcoma viral oncogene homolog | NM_000222 | 4557694 | KIT |
| | (KIT), mRNA | | | |
| 383 | Homo sapiens ceruloplasmin (ferroxidase) (CP), mRNA | NM_000096 | 4557484 | CP |
| 384 | Homo sapiens ectonucleoside triphosphate diphosphohydrolase 5 (ENTPD5), | NM_001249 | 4557426 | ENTPD5 |
| | mRNA | | | |
| 385 | Homo sapiens per1-like domain containing 1 (PERLD1), mRNA | NM_033419 | 45505179 | PERLD1 |
| 386 | Homo sapiens peptidylprolyl isomerase A (cyclophilin A) (PPIA), transcript variant | NM_021130 | 45439309 | PPIA |
| | 1, mRNA | | | |
| 387 | Homo sapiens C-type lectin domain family 3, member B (CLEC3B), mRNA | NM_003278 | 4507556 | CLEC3B |
| 388 | Homo sapiens transgelin 2 (TAGLN2), mRNA | NM_003564 | 4507356 | TAGLN2 |
| 389 | Homo sapiens spectrin, beta, non-erythrocytic 1 (SPTBN1), transcript variant 1, | NM_003128 | 4507194 | SPTBN1 |
| | mRNA | | | |
| 390 | Homo sapiens spectrin, alpha, non-erythrocytic 1 (alpha-fodrin) (SPTAN1), mRNA | NM_003127 | 4507190 | SPTAN1 |
| 391 | Homo sapiens presenilin 2 (Alzheimer disease 4) (PSEN2), transcript variant 1, | NM_000447 | 4506164 | PSEN2 |
| | mRNA | | | |
| 392 | Homo sapiens protective protein for beta-galactosidase (galactosialidosis) (PPGB), | NM_000308 | 4505988 | PPGB |
| | mRNA | | | |
| 393 | Homo sapiens polymerase (RNA) II (DNA directed) polypeptide 8, 140 kDa | NM_000938 | 4505940 | RNA |
| | (POLR2B), mRNA | | | |
| 394 | Homo sapiens dynein, cytoplasmic, light polypeptide 1 (DNCL1), mRNA | NM_003746 | 4505812 | DNCL1 |
| 395 | Homo sapiens msh homeo box homolog 1 (Drosophila) (MSX1), mRNA | NM_002448 | 4505266 | MSX1 |
| 396 | Homo sapiens latent transforming growth factor beta binding protein 4 (LTBP4), | NM_003573 | 4505036 | LTBP4 |
| | mRNA | | | |
| 397 | Homo sapiens junction plakoglobin (JUP), transcript variant 1, mRNA | NM_002230 | 4504810 | JUP |
| 398 | Homo sapiens heat shock 10 kDa protein 1 (chaperonin 10) (HSPE1), mRNA | NM_002157 | 4504522 | HSPE1 |
| 400 | Homo sapiens glioblastoma amplified sequence (GBAS), mRNA | NM_001483 | 4503936 | GBAS |
| 402 | Homo sapiens erythrocyte membrane protein band 4.1-like 2 (EPB41L2), mRNA | NM_001431 | 4503578 | EPB41L2 |
| 403 | Homo sapiens eukaryotic translation initiation factor 4 gamma, 2 (EIF4G2), mRNA | NM_001418 | 4503538 | EIF4G2 |
| 404 | Homo sapiens eukaryotic translation initiation factor 4A, isoform 1 (EIF4A1), | NM_001416 | 4503528 | EIF4A1 |
| | mRNA | | | |
| 405 | Homo sapiens heparin-binding EGF-like growth factor (HBEGF), mRNA | NM_001945 | 4503412 | HBEGF |
| 406 | Homo sapiens ADP-ribosylation factor 3 (ARF3), mRNA | NM_001659 | 4502202 | ARF3 |
| 408 | Homo sapiens Meis1, myeloid ecotropic viral integration site 1 homolog (mouse) | NM_002398 | 45006902 | MEIS1 |
| | (MEIS1), mRNA | | | |
| 409 | Homo sapiens v-jun sarcoma virus 17 oncogene homolog (avian) (JUN), mRNA | NM_002228 | 44890066 | JUN |
| 410 | Homo sapiens peptidylprolyl isomerase B (cyclophilin B) (PPIB), mRNA | NM_000942 | 44890060 | PPIB |
| 411 | Homo sapiens caldesmon 1 (CALD1), transcript variant 1, mRNA | NM_033138 | 44680104 | CALD1 |
| 412 | Homo sapiens ADP-ribosylation factor-like 2 binding protein (ARL2BP), mRNA | NM_012106 | 42794776 | ARL2BP |
| 413 | Homo sapiens thyroid hormone responsive (SPOT14 homolog, rat) (THRSP), | NM_003251 | 42764629 | THRSP |
| | mRNA | | | |
| 414 | Homo sapiens ATP-binding cassette, sub-family B (MDR/TAP), member 1 | NM_000927 | 42741658 | MDR/TAP |
| | (ABCB1), mRNA | | | |
| 415 | PREDICTED: Homo sapiens KIAA1458 protein (KIAA1458), mRNA | XM_044434 | 42657086 | KIAA1458 |
| 416 | Homo sapiens lectin, galactoside-binding, soluble, 8 (galectin 8) (LGALS8), | NM_201543 | 42544186 | LGALS8 |
| | transcript variant 2, mRNA | | | |
| 417 | Homo sapiens heat shock 105 kDa/110 kDa protein 1 (HSPH1), mRNA | NM_006644 | 42544158 | HSPH1 |
| 418 | Homo sapiens CCAAT/enhancer binding protein zeta (CEBPZ), mRNA | NM_005760 | 42542391 | CEBPZ |
| 419 | Homo sapiens signal transducer and activator of transcription 5B (STAT5B), | NM_012448 | 42519913 | STAT5B |
| | mRNA | | | |
| 420 | Homo sapiens thyroid hormone receptor interactor 10 (TRIP10), mRNA | NM_004240 | 42516568 | TRIP10 |
| 422 | Homo sapiens reticulon 3 (RTN3), transcript variant 2, mRNA | NM_201428 | 41393607 | RTN3 |
| 423 | Homo sapiens complement component 1, s subcomponent (C1S), transcript | NM_201442 | 41393601 | C1S |
| | variant 2, mRNA | | | |
| 424 | Homo sapiens 3-hydroxyisobutyrate dehydrogenase (HIBADH), mRNA | NM_152740 | 41393565 | HIBADH |
| 425 | Homo sapiens RAB5C, member RAS oncogene family (RAB5C), transcript variant | NM_004583 | 41393544 | RAB5C |
| | 2, mRNA | | | |
| 426 | Homo sapiens SEC31-like 1 (S. cerevisiae) (SEC31L1), transcript variant 1, mRNA | NM_014933 | 41349438 | SEC31L1 |
| 428 | Homo sapiens clathrin, heavy polypeptide (Hc) (CLTC), mRNA | NM_004859 | 41327727 | CLTC |
| 429 | Homo sapiens calpain 7 (CAPN7), mRNA | NM_014296 | 41327720 | CAPN7 |
| 430 | Homo sapiens UDP glucuronosyltransferase 1 family, polypeptide A3 (UGT1A3), | NM_019093 | 41282228 | UGT1A3 |
| | mRNA | | | |
| 431 | Homo sapiens prostaglandin F2 receptor negative regulator (PTGFRN), mRNA | NM_020440 | 41152505 | PTGFRN |
| 432 | Homo sapiens DnaJ (Hsp40) homolog, subfamily C, member 19 (DNAJC19), | NM_145261 | 41152082 | DNAJC19 |
| | transcript variant 1, mRNA | | | |
| 433 | Homo sapiens steroid sensitive gene 1 (URB), transcript variant 2, mRNA | NM_199512 | 41152075 | URB |
| 434 | Homo sapiens kinesin 2 60/70 kDa (KNS2), transcript variant 2, mRNA | NM_182923 | 41055203 | KNS2 |
| 435 | Homo sapiens protein regulator of cytokinesis 1 (PRC1), transcript variant 1, | NM_003981 | 40807441 | PRC1 |
| | mRNA | | | |
| 436 | Homo sapiens dihydrouridine synthase 1-like (S. cerevisiae) (DUS1L), mRNA | NM_022156 | 40807365 | DUS1L |
| 437 | Homo sapiens RAB7, member RAS oncogene family (RAB7), mRNA | NM_004637 | 40807361 | RAB7 |
| 438 | Homo sapiens ubiquitin-conjugating enzyme E2 variant 1 (UBE2V1), transcript | NM_021988 | 40806163 | UBE2V1 |
| | variant 1, mRNA | | | |
| 439 | Homo sapiens prolylcarboxypeptidase (angiotensinase C) (PRCP), transcript | NM_005040 | 40805839 | PRCP |
| | variant 1, mRNA | | | |
| 440 | Homo sapiens chemokine (C—X—C motif) ligand 12 (stromal cell-derived factor 1) | NM_000609 | 40316922 | CXCL12 |
| | (CXCL12), transcript variant 1, mRNA | | | |
| 441 | Homo sapiens serum amyloid A1 (SAA1), transcript variant 2, mRNA | NM_199161 | 40316909 | SAA1 |
| 442 | Homo sapiens inter-alpha (globulin) inhibitor H4 (plasma Kallikrein-sensitive | NM_002218 | 40288189 | ITIH4 |
| | glycoprotein) (ITIH4), mRNA | | | |
| 443 | Homo sapiens inositol 1,4,5-trisphosphate 3-kinase C (ITPKC), mRNA | NM_025194 | 40254974 | ITPKC |
| 444 | Homo sapiens mediator of RNA polymerase II transcription, subunit 4 homolog | NM_014166 | 40254874 | MED4 |
| | (yeast) (MED4), mRNA | | | |
| 445 | Homo sapiens catenin (cadherin-associated protein), beta 1, 88 kDa (CTNNB1), | NM_001904 | 40254459 | CTNNB1 |
| | mRNA | | | |
| 446 | Homo sapiens fucosidase, alpha-L-2, plasma (FUCA2), mRNA | NM_032020 | 40068511 | FUCA2 |
| 447 | Homo sapiens brix domain containing 1 (BXDC1), mRNA | NM_032194 | 39930468 | BXDC1 |
| 448 | Homo sapiens NADH dehydrogenase (ubiquinone) Fe—S protein 6, 13 kDa (NADH- | NM_004553 | 39812335 | NDUFS6 |
| | coenzyme Q reductase) (NDUFS6), mRNA | | | |
| 449 | Homo sapiens hypothetical protein FLJ10534 (FLJ10534), mRNA | NM_018128 | 39780587 | FLJ10534 |
| 450 | Homo sapiens taxilin alpha (TXLNA), mRNA | NM_175852 | 39725959 | TXLNA |
| 451 | Homo sapiens vascular endothelial growth factor B (VEGFB), mRNA | NM_003377 | 39725673 | VEGFB |
| 452 | Homo sapiens solute carrier family 35, member A4 (SLC35A4), mRNA | NM_080670 | 39725666 | SLC35A4 |
| 453 | Homo sapiens tropomyosin 3 (TPM3), transcript variant 2, mRNA | NM_153649 | 39725631 | TPM3 |
| 454 | Homo sapiens tyrosine 3-monooxygenase/tryptophan 5-monooxygenase activation | BC063824 | 39645796 | |
| | protein, zeta polypeptide, transcript variant 2, mRNA (cDNA clone MGC: 74590 | | | |
| | IMAGE: 6180974), complete cds | | | |
| 455 | Homo sapiens cerebion (CRBN), mRNA | NM_016302 | 39545579 | CRBN |
| 456 | Homo sapiens tensin like C1 domain containing phosphatase (tensin 2) (TENC1), | NM_015319 | 38787940 | TENC1 |
| | transcript variant 1, mRNA | | | |
| 458 | Homo sapiens serine/threonine kinase 6 (STK6), transcript variant 5, mRNA | NM_198436 | 38327569 | STK6 |
| 459 | Homo sapiens ubiquitin-conjugating enzyme E2L 3 (UBE2L3), transcript variant 1, | NM_003347 | 38157977 | UBE2L3 |
| | mRNA | | | |
| 460 | Homo sapiens small nuclear ribonucleoprotein polypeptides B and B1 (SNRPB), | NM_003091 | 38149990 | SNRPB |
| | transcript variant 2, mRNA | | | |
| 461 | Homo sapiens non-metastatic cells 1, protein (NM23A) expressed in (NME1), | NM_198175 | 38045912 | NM23A |
| | transcript variant 1, mRNA | | | |
| 462 | Homo sapiens gelsolin (amyloidosis, Finnish type) (GSN), transcript variant 1, | NM_000177 | 38016941 | GSN |
| | mRNA | | | |
| 463 | Homo sapiens LP4947 mRNA, complete cds | AY203960 | 37782471 | |
| 464 | Homo sapiens basic leucine zipper nuclear factor 1 (JEM-1) (BLZF1), mRNA | NM_003666 | 37622354 | BLZF1 |
| 466 | Homo sapiens ATPase, H+ transporting, lysosomal accessory protein 1 | NM_001183 | 37577154 | ATP6AP1 |
| | (ATP6AP1), mRNA | | | |
| 467 | Homo sapiens UBBP4 pseudogene for ubiquitin UBB | X07499 | 37572 | |
| 468 | Homo sapiens eukaryotic translation initiation factor 5 (EIF5), transcript variant 1, | NM_001969 | 37537714 | EIF5 |
| | mRNA | | | |
| 469 | Homo sapiens hypothetical protein LOC196463 (LOC196463), mRNA | NM_173542 | 37059775 | LOC196463 |
| 470 | Homo sapiens vacuolar protein sorting 25 (yeast) (VPS25), mRNA | NM_032353 | 37059727 | VPS25 |
| 471 | Homo sapiens RP42 homolog (RP42), mRNA | NM_020640 | 36030882 | RP42 |
| 472 | Homo sapiens ribophorin II (RPN2), mRNA | NM_002951 | 35493915 | RPN2 |
| 473 | Homo sapiens galectin-3 internal gene (GALIG), mRNA | NM_194327 | 34996518 | GALIG |
| 474 | Human binding protein mRNA, 3′end | L23114 | 349469 | |
| 475 | Homo sapiens Kruppel-like factor 4 (gut) (KLF4), mRNA | NM_004235 | 34916057 | KLF4 |
| 476 | cr: gi\|34783417\|gb\|BC030698.2\|Homo sapiens a disintegrin and metalloproteinase | BC030698 | 34783417 | |
| | domain 32, mRNA (cDNA clone MGC: 26855 IMAGE: 4822988), complete cds | | | |
| 478 | Homo sapiens cDNA FLJ44539 fis, clone UTERU3005205 | AK126503 | 34533004 | |
| 479 | Homo sapiens cDNA FLJ42531 fis, clone BRACE3002508, highly similar to Homo | AK124522 | 34530327 | WDR11 |
| | sapiens WD repeat domain 11 (WDR11) | | | |
| 480 | Homo sapiens cDNA FLJ16619 fis, clone TESTI4014553, highly similar to Homo | AK122930 | 34528375 | |
| | sapiens high-risk human papilloma viruses E6 oncoproteins targeted protein | | | |
| | E6TP1 alpha | | | |
| 481 | Homo sapiens cDNA FLJ16383 fis, clone TLIVE2007016, highly similar to | AK122805 | 34528002 | |
| | NUCLEOLIN | | | |
| 483 | Homo sapiens thyrotrophic embryonic factor (TEF), mRNA | NM_003216 | 34486096 | TEF |
| 484 | Homo sapiens bromodomain containing 8 (BRD8), transcript variant 1, mRNA | NM_006696 | 34452704 | BRD8 |
| 485 | Homo sapiens ARP3 actin-related protein 3 homolog (yeast) (ACTR3), mRNA | NM_005721 | 34452698 | ACTR3 |
| 486 | Homo sapiens mRNA; cDNA DKFZp686F14109 (from clone DKFZp686F14109) | BX649148 | 34368320 | |
| 487 | Homo sapiens mRNA; cDNA DKFZp686K17155 (from clone DKFZp686K17155) | BX648472 | 34367634 | |
| 488 | Homo sapiens discs, large (Drosophila) homolog-associated protein 4 (DLGAP4), | NM_014902 | 34335252 | DLGAP4 |
| | transcript variant 1, mRNA | | | |
| 489 | Homo sapiens coatomer protein complex, subunit beta (COPB), mRNA | NM_016451 | 34222324 | COPB |
| 490 | Homo sapiens PRP19/PSO4 pre-mRNA processing factor 19 homolog (S. cerevisiae) | NM_014502 | 34222313 | PRPF19 |
| | (PRPF19), mRNA | | | |
| 491 | Homo sapiens family with sequence similarily 82, member A, mRNA (cDNA clone | BC024243 | 34193710 | |
| | MGC: 33318 IMAGE: 4798767), complete cds | | | |
| 492 | Homo sapiens leucine rich repeat containing 45 (LRRC45), mRNA | NM_144999 | 34147703 | LRRC45 |
| 493 | Homo sapiens DNA methyltransferase 1 associated protein 1 (DMAP1), mRNA | NM_019100 | 34147584 | DMAP1 |
| 494 | Homo sapiens RAB9A, member RAS oncogene family (RAB9A), mRNA | NM_004251 | 34147553 | RAB9A |
| 495 | Homo sapiens family with sequence similarity 44, member B (FAM44B), mRNA | NM_138389 | 34147528 | FAM44B |
| 496 | Homo sapiens serpin peptidase inhibitor, clade E (nexin, plasminogen activator | NM_006216 | 34147323 | SERPINE2 |
| | inhibitor type 1), member 2 (SERPINE2), mRNA | | | |
| 497 | Homo sapiens Fas apoptotic inhibitory molecule 2 (FAIM2), mRNA | NM_012306 | 34101289 | FAIM2 |
| 498 | Homo sapiens hypothetical protein MGC10744 (MGC10744), transcript variant 2, | NM_183065 | 34101277 | MGC10744 |
| | mRNA | | | |
| 499 | Homo sapiens Y box binding protein 1 (YBX1), mRNA | NM_004559 | 34098945 | YBX1 |
| 500 | Homo sapiens PRP39 pre-mRNA processing factor 39 homolog (yeast) (PRPF39), | NM_017922 | 33948298 | PRPF39 |
| | mRNA | | | |
| 501 | Homo sapiens eukaryotic translation initiation factor 2B, subunit 5 epsilon, 82 kDa | NM_003907 | 33667022 | EIF2B5 |
| | (EIF2B5), mRNA | | | |
| 502 | Homo sapiens LSM4 homolog, U6 small nuclear RNA associated (S. cerevisiae) | NM_012321 | 33620778 | LSM4 |
| | (LSM4), mRNA | | | |
| 505 | Homo sapiens methionine adenosyltransferase II, beta (MAT2B), transcript variant | NM_013283 | 33519456 | MAT2B |
| | 1, mRNA | | | |
| 506 | Homo sapiens activating transcription factor 4 (tax-responsive enhancer element | NM_182810 | 33469973 | ATF4 |
| | B67) (ATF4), transcript variant 2, mRNA | | | |
| 507 | Homo sapiens RNA binding motif protein 12 (RBM12), transcript variant 1, mRNA | NM_006047 | 33469952 | RBM12 |
| 508 | Homo sapiens RAVER1 (RAVER1), mRNA | NM_133452 | 33457331 | RAVER1 |
| 509 | Homo sapiens EPH receptor A2 (EPHA2), mRNA | NM_004431 | 32967310 | EPHA2 |
| 510 | Homo sapiens carnitine deficiency-associated, expressed in ventricle 1 (CDV1), | NM_014055 | 32526900 | CDV1 |
| | transcript variant 1, mRNA | | | |
| 511 | Homo sapiens nucleolar protein 5A (56 kDa with KKE/D repeat) (NOL5A), mRNA | NM_006392 | 32483373 | NOL5A |
| 512 | Homo sapiens sestrin 2 (SESN2), mRNA | NM_031459 | 32454742 | SESN2 |
| 513 | Homo sapiens coiled-coil-helix-coiled-coil-helix domain containing 2 (CHCHD2), | NM_016139 | 32307179 | CHCHD2 |
| | mRNA | | | |
| 514 | Homo sapiens neuronatin (NNAT), transcript variant 2, mRNA | NM_181689 | 32307135 | NNAT |
| 515 | Homo sapiens ankyrin repeat domain 37 (ANKRD37), mRNA | NM_181726 | 32171200 | ANKRD37 |
| 516 | Homo sapiens inhibitor of DNA binding 3, dominant negative helix-loop-helix | NM_002167 | 32171181 | ID3 |
| | protein (ID3), mRNA | | | |
| 517 | Homo sapiens microtubule-associated protein 1 light chain 3 alpha (MAP1LC3A), | NM_032514 | 31563519 | MAP1LC3A |
| | transcript variant 1, mRNA | | | |
| 518 | Homo sapiens tyrosine kinase with immunoglobulin-like and EGF-like domains 1 | NM_005424 | 31543809 | TIE1 |
| | (TIE1), mRNA | | | |
| 519 | Homo sapiens splicing factor, arginine/serine-rich 1 (splicing factor 2, alternate | NM_006924 | 31543618 | SFRS1 |
| | splicing factor) (SFRS1), mRNA | | | |
| 520 | Homo sapiens integral membrane protein 2B (ITM2B), mRNA | NM_021999 | 31542987 | ITM2B |
| 521 | Homo sapiens Ras-related GTP binding C (RRAGC), mRNA | NM_022157 | 31542866 | RRAGC |
| 522 | Homo sapiens ring finger protein 185 (RNF185), mRNA | NM_152267 | 31542782 | RNF185 |
| 523 | Homo sapiens nuclear receptor subfamily 2, group F, member 2 (NR2F2), mRNA | NM_021005 | 31377723 | NR2F2 |
| 524 | Homo sapiens protein phosphatase 2, regulatory subunit B (B56), epsilon isoform | NM_006246 | 31083295 | PPP2R5E |
| | (PPP2R5E), mRNA | | | |
| 525 | Homo sapiens protein phosphatase 2, regulatory subunit B (B56), alpha isoform | NM_006243 | 30795205 | PPP2R5A |
| | (PPP2R5A), mRNA | | | |
| 526 | cr: gi\|30583882\|gb\|BT007522.1\|Synthetic construct Homo sapiens ubiquitin | BT007522 | 30583882 | |
| | specific protease 5 (isopeptidase T) mRNA, partial cds | | | |
| 527 | Homo sapiens KIAA0280 protein, mRNA (cDNA clone MGC: 50842 | BC052341 | 30481647 | |
| | IMAGE: 5757011), complete cds | | | |
| 528 | Homo sapiens late cornified envelope 1B (LCE1B), mRNA | NM_178349 | 30387655 | LCE1B |
| 529 | Homo sapiens protein tyrosine phosphatase domain containing 1 (PTPDC1), | NM_177995 | 30089949 | PTPDC1 |
| | transcript variant 2, mRNA | | | |
| 530 | Homo sapiens sodium channel, voltage-gated, type I, alpha (SCN1A), mRNA | NM_006920 | 29893558 | SCN1A |
| 531 | Homo sapiens transient receptor potential cation channel, subfamily M, member 7 | NM_017672 | 29893551 | TRPM7 |
| | (TRPM7), mRNA | | | |
| 532 | Homo sapiens golgi reassembly stacking protein 2, 55 kDa (GORASP2), mRNA | NM_015530 | 29826293 | GORASP2 |
| 533 | Homo sapiens AngRem46 mRNA, complete cds | AY040224 | 29646854 | |
| 534 | Homo sapiens myosin regulatory light chain MRLC2 (MRLC2), mRNA | NM_033546 | 29568092 | MRLC2 |
| 535 | Homo sapiens sarcoma antigen NY-SAR-77 mRNA, partial cds | AY211921 | 29164884 | PALLD |
| 537 | Homo sapiens proteasome (prosome, macropain) 26S subunit, non-ATPase, 11 | NM_002815 | 28872724 | PSMD11 |
| | (PSMD11), mRNA | | | |
| 538 | Homo sapiens metallothionein 1K (MT1K), mRNA | NM_176870 | 28866965 | MT1K |
| 539 | Homo sapiens polypyrimidine tract binding protein 1 (PTBP1), transcript variant 3, | NM_031991 | 28558996 | PTBP1 |
| | mRNA | | | |
| 540 | Homo sapiens adhesion regulating molecule 1 (ADRM1), transcript variant 2, | NM_175573 | 28373193 | ADRM1 |
| | mRNA | | | |
| 541 | Homo sapiens t-complex-associated-testis-expressed 3 (TCTE3), mRNA | NM_174910 | 28372534 | TCTE3 |
| 542 | Homo sapiens hemoglobin, epsilon 1 (HBE1), mRNA | NM_005330 | 28302129 | HBE1 |
| 543 | Homo sapiens RAB3A interacting protein (rabin3) (RAB3IP), transcript variant | NM_022456 | 28274698 | RAB3IP |
| | alpha 1, mRNA | | | |
| 544 | human full-length cDNA clone CS0DF038YO05 of Fetal brain of Homo sapiens | BX247988 | 28193159 | |
| | (human) | | | |
| 545 | Homo sapiens transforming, acidic coiled-coil containing protein 1, mRNA (cDNA | BC041391 | 27552854 | |
| | clone MGC: 43967 IMAGE: 5276767), complete cds | | | |
| 546 | Homo sapiens cDNA clone IMAGE: 5277710, containing frame-shift errors | BC042364 | 27503450 | |
| 547 | Homo sapiens coronin, actin binding protein, 1C (CORO1C), mRNA | NM_014325 | 27477119 | CORO1C |
| 548 | Homo sapiens lamin A/C (LMNA), transcript variant 1, mRNA | NM_170707 | 27436945 | LMNA |
| 549 | Homo sapiens colony stimulating factor 1 (macrophage) (CSF1), transcript variant | NM_172212 | 27262666 | CSF1 |
| | 4, mRNA | | | |
| 550 | Homo sapiens potassium large conductance calcium-activated channel, subfamily | NM_014505 | 26051274 | KCNMB4 |
| | M, beta member 4 (KCNMB4), mRNA | | | |
| 551 | Homo sapiens aldo-keto reductase family 1, member B1 (aldose reductase) | NM_001628 | 24497579 | AKR1B1 |
| | (AKR1B1), mRNA | | | |
| 552 | Homo sapiens aldo-keto reductase family 1, member A1 (aldehyde reductase) | NM_153326 | 24497576 | AKR1A1 |
| | (AKR1A1), transcript variant 2, mRNA | | | |
| 553 | Homo sapiens RAB11 family interacting protein 5 (class I) (RAB11FIP5), mRNA | NM_015470 | 24308074 | RAB11FIP5 |
| 554 | Homo sapiens coronin, actin binding protein, 2B (CORO2B), mRNA | NM_006091 | 24307902 | CORO2B |
| 555 | Homo sapiens hypothetical protein MGC39520 (MGC39520), mRNA | NM_153364 | 23503300 | MGC39520 |
| 556 | Homo sapiens zinc finger protein 23 (KOX 16) (ZNF23), mRNA | NM_145911 | 23308736 | ZNF23 |
| 557 | Homo sapiens sorting nexin 17 (SNX17), mRNA | NM_014748 | 23238249 | SNX17 |
| 558 | Homo sapiens microfibrillar-associated protein 4 (MFAP4), mRNA | NM_002404 | 23111004 | MFAP4 |
| 559 | Homo sapiens glutathione S-transferase M5 (GSTM5), mRNA | NM_000851 | 23065562 | GSTM5 |
| 560 | Homo sapiens actin related protein 2/3 complex, subunit 1B, 41 kDa (ARPC1B), | NM_005720 | 22907055 | ARPC1B |
| | mRNA | | | |
| 561 | Homo sapiens cDNA FLJ90785 fis, clone THYRO1001457, moderately similar to | AK075266 | 22761241 | |
| | H. sapiens mRNA for protein kinase C mu | | | |
| 562 | Homo sapiens peptidylprolyl isomerase (cyclophilin)-like 2 (PPIL2), transcript | NM_014337 | 22547209 | PPIL2 |
| | variant 1, mRNA | | | |
| 563 | Homo sapiens ubiquitin B (UBB), mRNA | NM_018955 | 22538474 | UBB |
| 564 | cand_Human_STR_62C3_CatepsinZ_26Jun2003.fa | NM_001336 | 22538441 | CTSZ |
| | BaseSame: gi\|22538441\|ref\|NM_001336.2\|Homo sapiens cathepsin Z (CTSZ), | | | |
| | mRNA | | | |
| 565 | Homo sapiens chromosome 9 open reading frame 24 (C9orf24), transcript variant | NM_147168 | 22219468 | C9orf24 |
| | 2, mRNA | | | |
| 566 | Homo sapiens popeye domain containing 2 (POPDC2), mRNA | NM_022135 | 22209003 | POPDC2 |
| 567 | Homo sapiens high mobility group AT-hook 1 (HMGA1), transcript variant 7, mRNA | NM_145905 | 22208978 | HMGA1 |
| 568 | Homo sapiens ankyrin repeat and SOCS box-containing 2 (ASB2), mRNA | NM_016150 | 22208962 | ASB2 |
| 569 | Homo sapiens pescadillo homolog 1, containing BRCT domain (zebrafish) (PES1), | NM_014303 | 22091458 | PES1 |
| | mRNA | | | |
| 570 | Homo sapiens pleckstrin homology-like domain, family B, member 2 (PHLDB2), | NM_145753 | 21955171 | PHLDB2 |
| | mRNA | | | |
| 571 | H. sapiens telomeric DNA sequence, clone 22QTEL021, read 22QTELOO021.seq | Z96504 | 2181579 | |
| 572 | Homo sapiens cDNA FLJ40520 fis, clone TESTI2046786, highly similar to | AK097839 | 21757726 | |
| | SYNDECAN-2 PRECURSOR | | | |
| 573 | Homo sapiens cDNA FLJ39365 fis, clone PEBLM2006049, highly similar to | AK096684 | 21756230 | |
| | Polymerase (RNA) III | | | |
| 574 | Homo sapiens cDNA FLJ38927 fis, clone NT2NE2012505, highly similar to Gallus | AK096246 | 21755688 | |
| | gallus mRNA for avena | | | |
| 575 | Homo sapiens cDNA FLJ37984 fis, clone CTONG2010652, weakly similar to | R617323 | 21754536 | |
| | THREONINE SYNTHASE (EC 4.2.99.2) | | | |
| 576 | Homo sapiens cDNA FLJ34101 fis, clone FCBBF3007631, highly similar to CAMP- | AK091420 | 21749785 | |
| | DEPENDENT PROTEIN KINASE, BETA-CATALYTIC SUBUNIT (EC 2.7.1.37) | | | |
| 577 | Homo sapiens acyl-Coenzyme A binding domain containing 5 (ACBD5), mRNA | NM_145698 | 21735486 | ACBD5 |
| 578 | Homo sapiens pyridoxine-5′-phosphate oxidase mRNA, complete cds | AF468030 | 21728335 | |
| 579 | Homo sapiens H-rev107-like protein 5, mRNA (cDNA clone MGC: 39540 | BC034222 | 21707988 | |
| | IMAGE: 5268703), complete cds | | | |
| 580 | Homo sapiens, Similar to pre-mRNA splicing SR protein rA4, clone | BC031898 | 21594702 | |
| | IMAGE: 4995698, mRNA | | | |
| 581 | Homo sapiens AXL receptor tyrosine kinase (AXL), transcript variant 1, mRNA | NM_021913 | 21536465 | AXL |
| 582 | Homo sapiens RNA binding protein, autoantigenic (hnRNP-associated with lethal | NM_007367 | 21396479 | RALY |
| | yellow homolog (mouse)) (RALY), transcript variant 2, mRNA | | | |
| 583 | Homo sapiens retinitis pigmentosa GTPase regulator interacting protein 1 | NM_020366 | 21361838 | RPGRIP1 |
| | (RPGRIP1), mRNA | | | |
| 584 | Homo sapiens chromosome 13 open reading frame 12 (C13orf12), mRNA | NM_015932 | 21361533 | C13orf12 |
| 585 | Homo sapiens Rac GTPase activating protein 1 (RACGAP1), mRNA | NM_013277 | 21361396 | RACGAP1 |
| 586 | Homo sapiens retinoblastoma-like 2 (p130) (RBL2), mRNA | NM_005611 | 21361291 | RBL2 |
| 587 | Homo sapiens acid phosphatase 6, lysophosphatidic (ACP6), mRNA | NM_016361 | 21359910 | ACP6 |
| 588 | Homo sapiens SH3-domain GRB2-like endophilin B1 (SH3GLB1), mRNA | NM_016009 | 21359904 | SH3GLB1 |
| 589 | Homo sapiens prothymosin, alpha (gene sequence 28) (PTMA), mRNA | NM_002823 | 21359859 | PTMA |
| 590 | Homo sapiens small nuclear ribonucleoprotein polypeptide G (SNRPG), mRNA | NM_003096 | 21359839 | SNRPG |
| 591 | Homo sapiens phosphoprotein enriched in astrocytes 15 (PEA15), mRNA | NM_003768 | 21359835 | PEA15 |
| 592 | Homo sapiens nucleosome assembly protein 1-like 1 (NAP1L1), transcript variant | NM_139207 | 21327707 | NAP1L1 |
| | 1, mRNA | | | |
| 593 | Homo sapiens DEAD (Asp-Glu-Ala-Asp) box polypeptide 41 (DDX41), mRNA | NM_016222 | 21071031 | DDX41 |
| 594 | Homo sapiens calmodulin 2 (phosphorylase kinase, delta) (CALM2), mRNA | NM_001743 | 20428653 | CALM2 |
| 595 | Homo sapiens ATPase, H+ transporting, lysosomal 13 kDa, V1 subunit G isoform 1 | NM_004888 | 20357534 | ATP6V1G1 |
| | (ATP6V1G1), mRNA | | | |
| 596 | Homo sapiens c-fos induced growth factor (vascular endothelial growth factor D) | NM_004469 | 19924297 | FIGF |
| | (FIGF), mRNA | | | |
| 597 | Homo sapiens DNA replication factor (CDT1), mRNA | NM_030928 | 19923847 | CDT1 |
| 598 | Homo sapiens HSPC244 (MGC13379), mRNA | NM_016499 | 19923799 | MGC13379 |
| 599 | Homo sapiens SUB1 homolog (S. cerevisiae) (SUB1), mRNA | NM_006713 | 19923783 | SUB1 |
| 600 | cand_Human_FGA_85C2_SEL1L_26Jun2003.fa | NM_005065 | 19923668 | SEL1L |
| | BaseSame: gi\|19923668\|ref\|NM_005065.3\|Homo sapiens sel-1 suppressor of lin- | | | |
| | 12-like (C. elegans) (SEL1L), mRNA | | | |
| 601 | Homo sapiens down-regulated by Ctnnb1, a (DRCTNNB1A), mRNA | NM_032581 | 19923645 | DRCTNNB1A |
| 602 | Homo sapiens echinoderm microtubule associated protein like 4 (EML4), mRNA | NM_019063 | 19923496 | EML4 |
| 603 | Homo sapiens RAB14, member RAS oncogene family (RAB14), mRNA | NM_016322 | 19923482 | RAB14 |
| 604 | Homo sapiens arginine-glutamic acid dipeptide (RE) repeats (RERE), mRNA | NM_012102 | 19923392 | RERE |
| 605 | Homo sapiens antigen identified by monoclonal antibody Ki-67 (MKI67), mRNA | NM_002417 | 19923216 | MKI67 |
| 606 | Homo sapiens ATPase, H+ transporting, lysosomal 16 kDa, V0 subunit c | NM_001694 | 19913436 | ATP6V0C |
| | (ATP6V0C), mRNA | | | |
| 607 | Homo sapiens integrin, beta 1 (fibronectin receptor, beta polypeptide, antigen | NM_002211 | 19743812 | ITGB1 |
| | CD29 includes MDF2, MSK12) (ITGB1), transcript variant 1A, mRNA | | | |
| 608 | H. sapiens mRNA for myosin-I beta | X98507 | 1926310 | |
| 609 | Homo sapiens Kell blood group precursor (McLeod phenotype) (XK), mRNA | NM_021083 | 18860914 | XK |
| 610 | Homo sapiens protein tyrosine phosphatase, receptor type, N (PTPRN), mRNA | NM_002846 | 18860905 | PTPRN |
| 611 | Homo sapiens stromal cell derived factor 4 (SDF4), mRNA | NM_016176 | 18699731 | SDF4 |
| 612 | Homo sapiens GNAS complex locus (GNAS), transcript variant 3, mRNA | NM_080425 | 18426897 | GNAS |
| 613 | Human somatic cytochrome c (HC1) processed pseudogene, complete cds | M22878 | 181256 | HC1 |
| 615 | Homo sapiens tRNA splicing endonuclease 34 homolog (SEN34, S. cerevisiae), | BC020805 | 18088780 | |
| | mRNA (cDNA clone MGC: 23828 IMAGE: 4277870), complete cds | | | |
| 616 | Homo sapiens tubulin, alpha 2 (TUBA2), transcript variant 1, mRNA | NM_006001 | 17921992 | TUBA2 |
| 617 | cand_Human_779_RTP_801_26Jun2003.fa | AF335324 | 17385764 | RTP801 |
| | BaseSame: gi\|17385764\|gb\|AF335324.1\|Homo sapiens RTP801 mRNA, complete | | | |
| | cds similar to rat RTP_801 | | | |
| 618 | Homo sapiens FBP-interacting repressor (FIR) mRNA, complete cds | AF217197 | 17152039 | FIR |
| 619 | Homo sapiens ret finger protein (RFP), transcript variant alpha, mRNA | NM_006510 | 17105396 | RFP |
| 620 | Human calcium-binding protein chp mRNA, complete cds | U61538 | 1706966 | |
| 621 | Homo sapiens cyclin D3 (CCND3), mRNA | NM_001760 | 16950657 | CCND3 |
| 622 | Homo sapiens cyclin D2 (CCND2), mRNA | NM_001759 | 16950656 | CCND2 |
| 624 | Homo sapiens profilin 1 (PFN1), mRNA | NM_005022 | 16753213 | PFN1 |
| 628 | Homo sapiens enolase 3 (beta, muscle) (ENO3), transcript variant 2, mRNA | NM_053013 | 16554591 | ENO3 |
| 629 | Homo sapiens cDNA FLJ25275 fis, clone STM06043 | AK058004 | 16554002 | |
| 630 | Homo sapiens cDNA FLJ32672 fis, clone TESTI1000131, highly similar to Homo | AK057234 | 16552844 | |
| | sapiens TCP11 mRNA | | | |
| 631 | Homo sapiens cDNA FLJ32321 fis, clone PROST2003563, highly similar to | AK056883 | 16552404 | |
| | AMYLOID-LIKE PROTEIN 2 PRECURSOR | | | |
| 632 | Homo sapiens cDNA FLJ31860 fis, clone NT2RP7001306, weakly similar to U1 | AK056422 | 16551821 | |
| | SMALL NUCLEAR RIBONUCLEOPROTEIN 70 KDA | | | |
| 633 | Homo sapiens enolase 1, (alpha) (ENO1), mRNA | NM_001428 | 16507965 | ENO1 |
| 634 | Homo sapiens MGC16028 similar to RIKEN cDNA 1700019E19 gene | NM_052873 | 16418376 | MGC16028 |
| | (MGC16028), mRNA | | | |
| 635 | Homo sapiens cell division cycle 42 (GTP binding protein, 25 kDa) (CDC42), | NM_001791 | 16357470 | CDC42 |
| | transcript variant 1, mRNA | | | |
| 636 | Homo sapiens eukaryotic translation initiation factor 3, subunit 8, 110 kDa, mRNA | BC001571 | 16306764 | |
| | (cDNA clone MGC: 5500 IMAGE: 3452953), complete cds | | | |
| 638 | Homo sapiens keratin 14 (epidermolysis bullosa simplex, Dowling-Meara, | NM_000526 | 15431309 | KRT14 |
| | Koebner) (KRT14), mRNA | | | |
| 641 | Homo sapiens interferon-stimulated transcription factor 3, gamma 48 kDa, mRNA | BC012077 | 15082337 | |
| | (cDNA clone MGC: 19975 IMAGE: 4653017), complete cds | | | |
| 642 | Homo sapiens nucleolar protein family A, member 3 (H/ACA small nucleolar | NM_018648 | 15011920 | NOLA3 |
| | RNPs) (NOLA3), mRNA | | | |
| 643 | Homo sapiens Williams-Beuren syndrome chromosome region 1 (WBSCR1), | NM_031992 | 14702179 | WBSCR1 |
| | transcript variant 2, mRNA | | | |
| 644 | Homo sapiens polymerase (RNA) II (DNA directed) polypeptide F (POLR2F), | NM_021974 | 14602451 | RNA |
| | mRNA | | | |
| 646 | Homo sapiens disrupted in renal carcinoma 2 (DIRC2), mRNA | NM_032839 | 14249551 | DIRC2 |
| 647 | Homo sapiens GLIS family zinc finger 2 (GLIS2), mRNA | NM_032575 | 14211890 | GLIS2 |
| 648 | Homo sapiens tubulin, beta 6 (TUBB6), mRNA | NM_032525 | 14210535 | TUBB6 |
| 649 | Homo sapiens heterogeneous nuclear ribonucleoprotein K (HNRPK), transcript | NM_002140 | 14165438 | HNRPK |
| | variant 1, mRNA | | | |
| 650 | Homo sapiens hypothetical protein FLJ11301 (FLJ11301), mRNA | NM_018385 | 14149719 | FLJ11301 |
| 651 | Homo sapiens KIAA1049 protein (KIAA1049), mRNA | NM_014972 | 14149656 | KIAA1049 |
| 652 | Homo sapiens heterogeneous nuclear ribonucleoprotein C (C1/C2) (HNRPC), | NM_031314 | 14110427 | HNRPC |
| | transcript variant 1, mRNA | | | |
| 653 | Homo sapiens heterogeneous nuclear ribonucleoprotein D-like (HNRPDL), | NM_031372 | 14110406 | HNRPDL |
| | transcript variant 2, mRNA | | | |
| 654 | Homo sapiens spinster (SPIN1), mRNA | NM_032038 | 14042967 | SPIN1 |
| 655 | Homo sapiens sirtuin (silent mating type information regulation 2 homolog) 2 (S. cerevisiae) | NM_012237 | 13775599 | SIRT2 |
| | (SIRT2), transcript variant 1, mRNA | | | |
| 656 | Homo sapiens DEAD (Asp-Glu-Ala-Asp) box polypeptide 5 (DDX5), mRNA | NM_004396 | 13514826 | DDX5 |
| 657 | Homo sapiens Mak3 homolog (S. cerevisiae) (MAK3), mRNA | NM_025146 | 13376734 | MAK3 |
| 658 | Homo sapiens chromosome 10 open reading frame 119 (C10orf119), mRNA | NM_024834 | 13376242 | C10orf119 |
| 659 | Homo sapiens STEAP family member 4 (STEAP4), mRNA | NM_024636 | 13375867 | STEAP4 |
| 660 | Homo sapiens steroid 5 alpha-reductase 2-like (SRD5A2L), mRNA | NM_024592 | 13375784 | SRD5A2L |
| 661 | Homo sapiens chromosome 12 open reading frame 8 (C12orf8), mRNA | NM_006817 | 13124889 | C12orf8 |
| 662 | Homo sapiens tubulointerstitial nephritis antigen-like 1 (TINAGL1), mRNA | NM_022164 | 11545917 | TINAGL1 |
| 663 | Homo sapiens calcium binding atopy-related autoantigen 1 (CBARA1), mRNA | NM_006077 | 11386184 | CBARA1 |
| 665 | Homo sapiens mannose-6-phosphate receptor (cation dependent) (M6PR), mRNA | NM_002355 | 10947032 | M6PR |
| 666 | Homo sapiens jun D proto-oncogene (JUND), mRNA | NM_005354 | 10938013 | JUND |
| 667 | Homo sapiens lectin, mannose-binding, 1 (LMAN1), mRNA | NM_005570 | 10862689 | LMAN1 |
| 668 | Human RATS1 mRNA, complete cds | U37688 | 1041013 | |
| 669 | Homo sapiens cytokine induced apoptosis inhibitor 1 (CIAPIN1), mRNA | NM_020313 | 10092672 | CIAPIN1 |
| 670 | Homo sapiens small muscle protein, X-linked (SMPX), mRNA | NM_014332 | 10047089 | SMPX |
| 673 | Homo sapiens PEST-containing nuclear protein (PCNP), mRNA | NM_020357 | 9966826 | PCNP |
| 674 | Homo sapiens ariadne homolog, ubiquitin-conjugating enzyme E2 binding protein, | NM_005744 | 9966762 | ARIH1 |
| | 1 (Drosophila) (ARIH1), mRNA | | | |
| 675 | Homo sapiens ATP-binding cassette, sub-family B (MDR/TAP), member 4 | NM_000443 | 9961253 | MDR/TAP |
| | (ABCB4), transcript variant A, mRNA | | | |
| 678 | Homo sapiens paired box gene 5 (B-cell lineage specific activator) (PAX5), mRNA | NM_016734 | 9951919 | PAX5 |
| 679 | Homo sapiens S-adenosylhomocysteine hydrolase (AHCY), mRNA | NM_000687 | 9951914 | AHCY |
| 680 | Homo sapiens G protein-coupled receptor 162 (GPR162), transcript variant A-2, | NM_019858 | 9951912 | GPR162 |
| | mRNA | | | |
| 681 | Homo sapiens laminin, gamma 1 (formerly LAMB2) (LAMC1), mRNA | NM_002293 | 9845497 | LAMC1 |
| 682 | Homo sapiens growth arrest and DNA-damage-inducible, gamma (GADD45G), | NM_006705 | 9790905 | GADD45G |
| | mRNA | | | |
| 683 | Homo sapiens protein phosphatase 1, regulatory (inhibitor) subunit 15A | NM_014330 | 9790902 | PPP1R15A |
| | (PPP1R15A), mRNA | | | |
| 684 | Homo sapiens microfibrillar-associated protein 2 (MFAP2), transcript variant 2, | NM_002403 | 9665260 | MFAP2 |
| | mRNA | | | |
| 685 | Homo sapiens similar to ubiquitin binding protein (UBPH), mRNA | NM_019116 | 9507222 | UBPH |
| 686 | Homo sapiens TERA mRNA, complete cds | AF212220 | 9437506 | |
| 687 | Homo sapiens telomeric repeat binding factor (NIMA-interacting) 1 (TERF1), | NM_017489 | 9257245 | TERF1 |
| | transcript variant 1, mRNA | | | |
| 688 | Homo sapiens succinate dehydrogenase complex, subunit B, iron sulfur (Ip) | NM_003000 | 9257241 | SDHB |
| | (SDHB), mRNA | | | |
| 689 | Homo sapiens peroxisomal membrane protein 2, 22 kDa (PXMP2), mRNA | NM_018663 | 8923891 | PXMP2 |
| 690 | Homo sapiens hypothetical protein FLJ20625 (FLJ20625), mRNA | NM_017907 | 8923578 | FLJ20625 |
| 691 | Homo sapiens timeless-interacting protein (FLJ20516), mRNA | NM_017858 | 8923484 | FLJ20516 |
| 692 | Homo sapiens RAB20, member RAS oncogene family (RAB20), mRNA | NM_017817 | 8923400 | RAB20 |
| 693 | Homo sapiens hypothetical protein FLJ20425 (LYAR), mRNA | NM_017816 | 8923397 | LYAR |
| 694 | Homo sapiens coiled-coil-helix-coiled-coil-helix domain containing 3 (CHCHD3), | NM_017812 | 8923389 | CHCHD3 |
| | mRNA | | | |
| 695 | Homo sapiens pyroglutamyl-peptidase I (PGPEP1), mRNA | NM_017712 | 8923197 | PGPEP1 |
| 696 | Homo sapiens hypothetical protein FLJ11046 (FLJ11046), mRNA | NM_018309 | 8922841 | FLJ11046 |
| 697 | Homo sapiens chromosome 1 open reading frame 106 (C1orf106), mRNA | NM_018265 | 8922753 | C1orf106 |
| 698 | Homo sapiens signal transducer and activator of transcription 3 interacting protein | NM_018255 | 8922734 | STATIP1 |
| | 1 (STATIP1), mRNA | | | |
| 699 | Homo sapiens chromosome 1 open reading frame 75 (C1orf75), mRNA | NM_018252 | 8922727 | C1orf75 |
| 700 | Homo sapiens CNDP dipeptidase 2 (metallopeptidase M20 family) (CNDP2), | NM_018235 | 8922698 | CNDP2 |
| | mRNA | | | |
| 701 | Homo sapiens chromosome 20 open reading frame 31 (C20orf31), mRNA | NM_018217 | 8922666 | C20orf31 |
| 702 | Homo sapiens SG2NA beta isoform mRNA, partial cds | AF243424 | 8895998 | |
| 703 | Homo sapiens endozepine-like protein type 2 mutant mRNA, complete cds | AF229804 | 8777848 | |
| 704 | Homo sapiens aconitase 1, soluble (ACO1), mRNA | NM_002197 | 8659554 | ACO1 |
| 705 | Homo sapiens tumor protein p53 (Li-Fraumeni syndrome) (TP53), mRNA | NM_000546 | 8400737 | TP53 |
| 706 | Homo sapiens retinol binding protein 1, cellular (RBP1), mRNA | NM_002899 | 8400726 | RBP1 |
| 707 | Homo sapiens par-6 partitioning defective 6 homolog alpha (C. elegans) | NM_016948 | 8394416 | PARD6A |
| | (PARD6A), mRNA | | | |
| 708 | Homo sapiens UDP-N-acetyl-alpha-D-galactosamine:polypeptide N- | NM_017423 | 8393408 | GALNT7 |
| | acetylgalactosaminyltransferase 7 (GalNAc-T7) (GALNT7), mRNA | | | |
| 709 | Homo sapiens albumin (ALB), mRNA | NM_000477 | 8392890 | ALB |
| 710 | Homo sapiens endophilin B1 mRNA, complete cds | AF263293 | 8118529 | |
| 711 | Homo sapiens KDEL (Lys-Asp-Glu-Leu) endoplasmic reticulum protein retention | NM_006854 | 8051609 | KDELR2 |
| | receptor 2 (KDELR2), mRNA | | | |
| 712 | Homo sapiens mRNA for KIAA1533 protein, partial cds | AB040966 | 7959332 | |
| 713 | Homo sapiens isoleucine-tRNA synthetase (IARS), transcript variant short, mRNA | NM_002161 | 7770073 | IARS |
| 714 | Homo sapiens sterile alpha motif and leucine zipper containing kinase AZK (ZAK), | NM_016653 | 7706600 | ZAK |
| | transcript variant 1, mRNA | | | |
| 715 | Homo sapiens GNAS complex locus (GNAS), transcript variant 4, mRNA | NM_016592 | 7706588 | GNAS |
| 716 | Homo sapiens TNF receptor-associated protein 1 (TRAP1), mRNA | NM_016292 | 7706484 | TRAP1 |
| 717 | Homo sapiens hematopoietic stem/progenitor cells 176 (HSPC176), mRNA | NM_016209 | 7706428 | HSPC176 |
| 718 | Homo sapiens ankyrin repeat and SOCS box-containing 4 (ASB4), transcript | NM_016116 | 7706378 | ASB4 |
| | variant 1, mRNA | | | |
| 719 | Homo sapiens brain protein 44-like (BRP44L), mRNA | NM_016098 | 7706368 | BRP44L |
| 720 | Homo sapiens coatomer protein complex, subunit zeta 1 (COPZ1), mRNA | NM_016057 | 7706336 | COPZ1 |
| 721 | Homo sapiens chromosome 14 open reading frame 166 (C14orf166), mRNA | NM_016039 | 7706321 | C14orf166 |
| 722 | Homo sapiens tumor necrosis factor receptor superfamily, member 12A | NM_016639 | 7706185 | TNFRSF12A |
| | (TNFRSF12A), mRNA | | | |
| 723 | Homo sapiens anterior pharynx defective 1 homolog A (C. elegans) (APH1A), | NM_016022 | 7705786 | APH1A |
| | mRNA | | | |
| 724 | Homo sapiens fumarylacetoacetate hydrolase domain containing 2A (FAHD2A), | NM_016044 | 7705607 | FAHD2A |
| | mRNA | | | |
| 725 | Homo sapiens lysosomal-associated membrane protein 1 (LAMP1), mRNA | NM_005561 | 7669500 | LAMP1 |
| 726 | Homo sapiens KIAA0494 (KIAA0494), mRNA | NM_014774 | 7662159 | KIAA0494 |
| 727 | Homo sapiens proteasome (prosome, macropain) 26S subunit, non-ATPase, 6 | NM_014814 | 7661913 | PSMD6 |
| | (PSMD6), mRNA | | | |
| 728 | Homo sapiens kinesin family member 14 (KIF14), mRNA | NM_014875 | 7661877 | KIF14 |
| 729 | Homo sapiens COMM domain containing 9 (COMMD9), mRNA | NM_014186 | 7661827 | COMMD9 |
| 730 | Homo sapiens cornichon homolog 4 (Drosophila) (CNIH4), mRNA | NM_014184 | 7661823 | CNIH4 |
| 731 | Homo sapiens REX2, RNA exonuclease 2 homolog (S. cerevisiae) (REXO2), | NM_015523 | 7661645 | REXO2 |
| | mRNA | | | |
| 732 | Homo sapiens vesicle-associated membrane protein 2 (synaptobrevin 2) (VAMP2), | NM_014232 | 7657674 | VAMP2 |
| | mRNA | | | |
| 733 | Homo sapiens solute carrier family 25, member 13 (citrin) (SLC25A13), mRNA | NM_014251 | 7657580 | SLC25A13 |
| 734 | Homo sapiens growth hormone inducible transmembrane protein (GHITM), mRNA | NM_014394 | 7657479 | GHITM |
| 735 | Homo sapiens LSM3 homolog, U6 small nuclear RNA associated (S. cerevisiae) | NM_014463 | 7657314 | LSM3 |
| | (LSM3), mRNA | | | |
| 737 | Homo sapiens nerve growth factor receptor (TNFRSF16) associated protein 1 | NM_014380 | 7657043 | TNFRSF16 |
| | (NGFRAP1), transcript variant 3, mRNA | | | |
| 738 | Homo sapiens claudin 15 (CLDN15), transcript variant 1, mRNA | NM_014343 | 7656980 | CLDN15 |
| 739 | Homo sapiens DnaJ (Hsp40) homolog, subfamily A, member 2 (DNAJA2), mRNA | NM_005880 | 7549807 | Hsp40 |
| 740 | Homo sapiens clone LCHN mRNA sequence | AF136629 | 7416935 | |
| 741 | Homo sapiens lymphocyte cytosolic protein 1 (L-plastin) (LCP1), mRNA | NM_002298 | 7382490 | LCP1 |
| 742 | Homo sapiens CD24 antigen (small cell lung carcinoma cluster 4 antigen) (CD24), | NM_013230 | 73623396 | CD24 |
| | mRNA | | | |
| 743 | Homo sapiens leukemia inhibitory factor receptor (LIFR), mRNA | NM_002310 | 73486667 | LIFR |
| 745 | Homo sapiens aldehyde dehydrogenase 3 family, member A2 (ALDH3A2), | NM_000382 | 73465985 | ALDH3A2 |
| | transcript variant 2, mRNA | | | |
| 746 | Homo sapiens procollagen C-endopeptidase enhancer (PCOLCE), mRNA | NM_002593 | 7262388 | PCOLCE |
| 747 | Homo sapiens similar to RIKEN cDNA B230118G17 gene (MGC19604), transcript | NM_001031734 | 72534753 | MGC19604 |
| | variant 1, mRNA | | | |
| 748 | Homo sapiens FERM domain containing 5 (FRMD5), transcript variant 1, mRNA | NM_001031729 | 72534751 | FRMD5 |
| 749 | Homo sapiens zinc finger protein 205 (ZNF205), transcript variant 1, mRNA | NM_001031686 | 72534663 | ZNF205 |
| 751 | Homo sapiens Rtf1, Paf1/RNA polymerase II complex component, homolog (S. cerevisiae) | NM_015138 | 72086156 | RTF1 |
| | (RTF1), mRNA | | | |
| 752 | Homo sapiens ATX1 antioxidant protein 1 homolog (yeast) (ATOX1), mRNA | NM_004045 | 72004264 | ATOX1 |
| 753 | Homo sapiens growth factor receptor-bound protein 14 (GRB14), mRNA | NM_004490 | 71979662 | GRB14 |
| 754 | Homo sapiens thyroid hormone receptor interactor 4 (TRIP4), mRNA | NM_016213 | 71773892 | TRIP4 |
| 760 | Homo sapiens LIM domain only 4 (LMO4), mRNA | NM_006769 | 7108354 | LMO4 |
| 761 | Homo sapiens centromere protein E, 312 kDa (CENPE), mRNA | NM_001813 | 71061467 | CENPE |
| 762 | Homo sapiens fibromodulin (FMOD), mRNA | NM_002023 | 71040110 | FMOD |
| 763 | Homo sapiens enoyl Coenzyme A hydratase 1, peroxisomal (ECH1), mRNA | NM_001398 | 70995210 | ECH1 |
| 764 | Homo sapiens cytochrome b5 reductase 3 (CYB5R3), transcript variant M, mRNA | NM_000398 | 70908370 | CYB5R3 |
| 765 | Homo sapiens fibrinogen gamma chain (FGG), transcript variant gamma-A, mRNA | NM_000509 | 70906436 | FGG |
| 766 | Homo sapiens fibrinogen beta chain (FGB), mRNA | NM_005141 | 70906434 | FGB |
| 767 | Homo sapiens ankyrin 1, erythrocytic (ANK1), transcript variant 5, mRNA | NM_020478 | 70780365 | ANK1 |
| 768 | Homo sapiens ankyrin 1, erythrocytic (ANK1), transcript variant 8, mRNA | NM_020481 | 70780362 | ANK1 |
| 769 | Homo sapiens ankyrin 1, erythrocytic (ANK1), transcript variant 2, mRNA | NM_020477 | 70780354 | ANK1 |
| 770 | Homo sapiens tumor protein D52 (TPD52), transcript variant 3, mRNA | NM_005079 | 70608192 | TPD52 |
| 771 | Homo sapiens tumor protein D52 (TPD52), transcript variant 1, mRNA | NM_001025252 | 70608171 | TPD52 |
| 772 | Homo sapiens cDNA FLJ20845 fis, clone ADKA01901 | AK000852 | 7021174 | |
| 773 | Homo sapiens serine threonine kinase 39 (STE20/SPS1 homolog, yeast) (STK39), | NM_013233 | 7019542 | STK39 |
| | mRNA | | | |
| 774 | Homo sapiens glutamate receptor, lonotropic, N-methyl D-aspartate-like 1A | NM_015532 | 70166742 | GRINL1A |
| | (GRINL1A), transcript variant 1, mRNA | | | |
| 775 | Homo sapiens glycyl-tRNA synthetase (GARS), mRNA | NM_002047 | 6996009 | GARS |
| 776 | Homo sapiens coatomer protein complex, subunit alpha (COPA), mRNA | NM_004371 | 6996002 | COPA |
| 777 | Homo sapiens ADP-ribosylation factor 5 (ARF5), mRNA | NM_001662 | 6995999 | ARF5 |
| 778 | Homo sapiens ADP-ribosylation factor 4 (ARF4), mRNA | NM_001660 | 6995998 | ARF4 |
| 779 | H. sapiens mRNA for peroxisomal C-terminal targeting signal import receptor | X84899 | 695565 | |
| 780 | Homo sapiens ubinuclein (UBN1) mRNA, alternatively spliced, complete cds | AF108460 | 6940750 | UBN1 |
| 782 | Homo sapiens nucleotide binding protein 2 (MinD homolog, E. coli) (NUBP2), | NM_012225 | 6912539 | NUBP2 |
| | mRNA | | | |
| 783 | Homo sapiens microtubule-associated protein, RP/EB family, member 1 | NM_012325 | 6912493 | MAPRE1 |
| | (MAPRE1), mRNA | | | |
| 784 | Homo sapiens integrin beta 1 binding protein (melusin) 2 (ITGB1BP2), mRNA | NM_012278 | 6912437 | ITGB1BP2 |
| 785 | Homo sapiens endothelial differentiation, lysophosphatidic acid G-protein-coupled | NM_012152 | 6912347 | EDG7 |
| | receptor, 7 (EDG7), mRNA | | | |
| 786 | Homo sapiens chromobox homolog 5 (HP1 alpha homolog, Drosophila) (CBX5), | NM_012117 | 6912291 | CBX5 |
| | mRNA | | | |
| 787 | Homo sapiens gap junction protein, alpha 7, 45 kDa (connexin 45) (GJA7), mRNA | NM_005497 | 69122472 | GJA7 |
| 788 | Homo sapiens chromosome 18 open reading frame 10 (C18orf10), mRNA | NM_015476 | 68534956 | C18orf10 |
| 789 | Homo sapiens PKCq-interacting protein PICOT (PICOT) mRNA, complete cds | AF118649 | 6840946 | PICOT |
| 790 | Homo sapiens CD34 antigen (CD34), transcript variant 1, mRNA | NM_001025109 | 68342037 | CD34 |
| 791 | Homo sapiens casein kinase 1, alpha 1 (CSNK1A1), transcript variant 2, mRNA | NM_001892 | 68303571 | CSNK1A1 |
| 792 | Homo sapiens chromosome 9 open reading frame 10 (C9orf10), mRNA | NM_014612 | 68299753 | C9orf10 |
| 793 | Homo sapiens keratin 8 (KRT8), mRNA | NM_002273 | 68226427 | KRT8 |
| 795 | Homo sapiens actin related protein 2/3 complex, subunit 4, 20 kDa (ARPC4), | NM_005718 | 68161505 | ARPC4 |
| | transcript variant 1, mRNA | | | |
| 796 | Homo sapiens secretogranin II (chromogranin C) (SCG2), mRNA | NM_003469 | 68160946 | SCG2 |
| 798 | Homo sapiens tubulin, beta, 2 (TUBB2), mRNA | NM_006088 | 68051719 | TUBB2 |
| 799 | Homo sapiens CD82 antigen (CD82), transcript variant 1, mRNA | NM_002231 | 67782352 | CD82 |
| 801 | Homo sapiens neutrophil cytosolic factor 2 (65 kDa, chronic granulomatous | NM_000433 | 67189969 | NCF2 |
| | disease, autosomal 2) (NCF2), mRNA | | | |
| 803 | Homo sapiens IMP (inosine monophosphate) dehydrogenase 2 (IMPDH2), mRNA | NM_000884 | 66933015 | IMPDH2 |
| 804 | Homo sapiens calnexin (CANX), transcript variant 2, mRNA | NM_001024649 | 66933004 | CANX |
| 805 | Homo sapiens zinc finger and BTB domain containing 16 (ZBTB16), transcript | NM_001018011 | 66932931 | ZBTB16 |
| | variant 2, mRNA | | | |
| 806 | Homo sapiens TM2 domain containing 2 (TM2D2), transcript variant 2, mRNA | NM_031940 | 66932892 | TM2D2 |
| 807 | Homo sapiens neuropilin 1 (NRP1), transcript variant 1, mRNA | NM_003873 | 66912183 | NRP1 |
| 808 | Homo sapiens signal peptidase complex subunit 1 homolog (S. cerevisiae) | NM_014041 | 66472393 | SPCS1 |
| | (SPCS1), mRNA | | | |
| 809 | Homo sapiens non-metastatic cells 2, protein (NM23B) expressed in (NME2), | NM_002512 | 66392201 | NM23B |
| | transcript variant 2, mRNA | | | |
| 810 | Homo sapiens HCLS1 associated protein X-1 (HAX1), transcript variant 1, mRNA | NM_006118 | 66363692 | HAX1 |
| 811 | Homo sapiens N-acetylglucosaminidase, alpha-(Sanfilippo disease IIIB) (NAGLU), | NM_000263 | 66346697 | NAGLU |
| | mRNA | | | |
| 812 | Homo sapiens fibrillin 2 (congenital contractural arachnodactyty) (FBN2), mRNA | NM_001999 | 66346694 | FBN2 |
| 813 | Homo sapiens dickkopf homolog 3 (Xenopus laevis) (DKK3), transcript variant 2, | NM_013253 | 66346687 | DKK3 |
| | mRNA | | | |
| 814 | Homo sapiens PAI-1 mRNA binding protein (PAI-RBP1), transcript variant 4, | NM_015640 | 66346684 | PAI-RBP1 |
| | mRNA | | | |
| 815 | Homo sapiens PAI-1 mRNA binding protein (PAI-RBP1), transcript variant 3, | NM_001018069 | 66346682 | PAI-RBP1 |
| | mRNA | | | |
| 816 | Homo sapiens PAI-1 mRNA binding protein (PAI-RBP1), transcript variant 1, | NM_001018067 | 66346678 | PAI-RBP1 |
| | mRNA | | | |
| 817 | Homo sapiens cathepsin B (CTSB), transcript variant 3, mRNA | NM_147781 | 66346648 | CTSB |
| 818 | Homo sapiens small EDRK-rich factor 2 (SERF2), mRNA | NM_001018108 | 66275523 | SERF2 |
| 819 | Homo sapiens GDP dissociation inhibitor 2 (GDI2), mRNA | NM_001494 | 6598322 | GDI2 |
| 820 | Homo sapiens signal sequence receptor, alpha (translocon-associated protein | NM_003144 | 6552340 | SSR1 |
| | alpha) (SSR1), mRNA | | | |
| 821 | Homo sapiens v-fos FBJ murine osteosarcoma viral oncogene homolog (FOS), | NM_005252 | 6552332 | FOS |
| | mRNA | | | |
| 822 | Homo sapiens activin A receptor, type IIA (ACVR2A), mRNA | NM_001616 | 65508448 | ACVR2A |
| 823 | Homo sapiens Wilms tumor 1 (WT1), transcript variant B, mRNA | NM_024424 | 65507816 | WT1 |
| 824 | Homo sapiens polymerase (RNA) III (DNA directed) polypeptide H (22.9 kD) | NM_001018050 | 65301160 | POLR3H |
| | (POLR3H), transcript variant 3, mRNA | | | |
| 825 | Homo sapiens lysophospholipase 3 (lysosomal phospholipase A2) (LYPLA3), | NM_012320 | 64085296 | LYPLA3 |
| | mRNA | | | |
| 826 | Homo sapiens GTP cyclohydrolase I feedback regulator (GCHFR), mRNA | NM_005258 | 6382072 | GCHFR |
| 827 | Homo sapiens tropomyosin 1 (alpha) (TPM1), transcript variant 5, mRNA | NM_000366 | 63252894 | TPM1 |
| 828 | Homo sapiens chaperonin containing TCP1, subunit 3 (gamma) (CCT3), transcript | NM_005998 | 63162571 | CCT3 |
| | variant 1, mRNA | | | |
| 829 | Homo sapiens enabled homolog (Drosophila), transcript variant 1, mRNA (cDNA | BC095481 | 63102460 | |
| | clone MGC: 111520 IMAGE: 30348815), complete cds | | | |
| 830 | Homo sapiens eukaryotic translation initiation factor 4 gamma, 3, mRNA (cDNA | BC094683 | 63102223 | |
| | clone MGC: 102792 IMAGE: 6188273), complete cds | | | |
| 831 | Homo sapiens cDNA clone MGC: 102952 IMAGE: 30529503, complete cds | BC094690 | 63101198 | |
| 832 | Homo sapiens optic atrophy 3 (autosomal recessive, with chorea and spastic | NM_001017989 | 63055069 | OPA3 |
| | paraplegia) (OPA3), transcript variant 1, mRNA | | | |
| 833 | Homo sapiens MBC3205 (UNQ501), mRNA | NM_198536 | 63055042 | UNQ501 |
| 834 | Homo sapiens actin, gamma 2, smooth muscle, enteric (ACTG2), mRNA | NM_001615 | 63054873 | ACTG2 |
| 835 | Homo sapiens transforming growth factor, beta 1 (Camurati-Engelmann disease) | NM_000660 | 63025221 | TGFB1 |
| | (TGFB1), mRNA | | | |
| 836 | Homo sapiens leucine rich repeat containing 10 (LRRC10), mRNA | NM_201550 | 63003902 | LRRC10 |
| 837 | Homo sapiens hypothetical protein BC017488 (LOC124446), mRNA | NM_194280 | 62988351 | LOC124446 |
| 838 | Homo sapiens mRNA for eukaryotic translation initiation factor 2, subunit 3 | AK222577 | 62896712 | |
| | gamma, 52 kDa variant, clone: CAE09969 | | | |
| 839 | Homo sapiens dual specificity phosphatase 5 (DUSP5), mRNA | NM_004419 | 62865889 | DUSP5 |
| 840 | Homo sapiens fatty acid binding protein 3, muscle and heart (mammary-derived | NM_004102 | 62865867 | FABP3 |
| | growth inhibitor) (FABP3), mRNA | | | |
| 841 | Homo sapiens TAF9 RNA polymerase II, TATA box binding protein (TBP)- | NM_001015892 | 62865613 | TBP |
| | associated factor, 32 kDa (TAF9), transcript variant 4, mRNA | | | |
| 842 | Homo sapiens ribophorin I (RPN1), mRNA | NM_002950 | 62739176 | RPN1 |
| 843 | Homo sapiens procollagen-lysine, 2-oxoglutarate 5-dioxygenase 2 (PLOD2), | NM_000935 | 62739165 | PLOD2 |
| | transcript variant 2, mRNA | | | |
| 844 | Homo sapiens procollagen-lysine, 2-oxoglutarate 5-dioxygenase 2 (PLOD2), | NM_182943 | 62739164 | PLOD2 |
| | transcript variant 1, mRNA | | | |
| 845 | Homo sapiens selenoprotein P, plasma, 1 (SEPP1), mRNA | NM_005410 | 62530390 | SEPP1 |
| 846 | Homo sapiens chromosome 6 open reading frame 82 (C6orf82), transcript variant | NM_001014840 | 62526025 | C6orf82 |
| | 5, mRNA | | | |
| 847 | Homo sapiens collapsin response mediator protein 1 (CRMP1), transcript variant | NM_001014809 | 62422570 | CRMP1 |
| | 1, mRNA | | | |
| 848 | Homo sapiens collapsin response mediator protein 1 (CRMP1), transcript variant | NM_001313 | 62422567 | CRMP1 |
| | 2, mRNA | | | |
| 849 | Homo sapiens profilin 2 (PFN2), transcript variant 1, mRNA | NM_053024 | 62422564 | PFN2 |
| 850 | Homo sapiens electron-transfer-flavoprotein, beta polypeptide (ETFB), transcript | NM_001985 | 62420878 | ETFB |
| | variant 1, mRNA | | | |
| 851 | Homo sapiens vimentin (VIM), mRNA | NM_003380 | 62414288 | VIM |
| 852 | Homo sapiens carboxypeptidase Z (CPZ), transcript variant 1, mRNA | NM_001014447 | 62388876 | CPZ |
| 853 | Homo sapiens modulator of estrogen induced transcription (FLJ13213), transcript | NM_001013843 | 62244003 | FLJ13213 |
| | variant 2, mRNA | | | |
| 854 | Homo sapiens insulin-like growth factor binding protein 4 (IGFBP4), mRNA | NM_001552 | 62243289 | IGFBP4 |
| 855 | Homo sapiens crystallin, mu (CRYM), transcript variant 1, mRNA | NM_001888 | 62241006 | CRYM |
| 856 | Homo sapiens carboxypeptidase X (M14 family) (CPXM), mRNA | NM_019609 | 62241005 | CPXM |
| 857 | Homo sapiens CD81 antigen (target of antiproliferative antibody 1) (CD81), mRNA | NM_004356 | 62240999 | CD81 |
| 858 | H. sapiens mRNA for skeletal muscle abundant protein 2 | X94234 | 62122100 | |
| 859 | Homo sapiens mRNA for LL5 beta protein variant protein | AB209903 | 62089391 | |
| 860 | Homo sapiens mRNA for CSDA protein variant protein | AB209896 | 62089377 | |
| 861 | Homo sapiens mRNA for delta isoform of regulatory subunit B56, protein | AB209861 | 62089307 | |
| | phosphatase 2A isoform 1 variant protein | | | |
| 862 | Homo sapiens mRNA for platelet-derived growth factor receptor beta precursor | AB209657 | 62088893 | |
| | variant protein | | | |
| 863 | Homo sapiens mRNA for heterogeneous nuclear ribonucleoprotein K isoform a | AB209562 | 62088703 | |
| | variant protein | | | |
| 864 | Homo sapiens mRNA for splicing factor, arginine/serine-rich 1 (splicing factor 2, | AB209558 | 62088695 | |
| | alternate splicing factor) variant protein | | | |
| 865 | Homo sapiens mRNA for Diaphanous 1 variant protein | AB209482 | 62088543 | |
| 866 | Homo sapiens mRNA for ankyrin 1 isoform 4 variant protein | AB209418 | 62088415 | |
| 867 | Homo sapiens mRNA for Neural cell adhesion molecule variant protein | AB209329 | 62088237 | |
| 868 | Homo sapiens mRNA for synaptotagmin binding, cytoplasmic RNA interacting | AB209098 | 62087775 | |
| | protein variant protein | | | |
| 869 | Homo sapiens fragile X mental retardation, autosomal homolog 1 (FXR1), | NM_001013438 | 61835163 | FXR1 |
| | transcript variant 2, mRNA | | | |
| 870 | Homo sapiens fragile X mental retardation, autosomal homolog 1 (FXR1), | NM_005087 | 61835147 | FXR1 |
| | transcript variant 1, mRNA | | | |
| 871 | Homo sapiens ataxia, cerebellar, Cayman type (caytaxin) (ATCAY), mRNA | NM_033064 | 61835122 | ATCAY |
| 872 | Homo sapiens solute carrier family 3 (activators of dibasic and neutral amino acid | NM_001013251 | 61744482 | SLC3A2 |
| | transport), member 2 (SLC3A2), transcript variant 6, mRNA | | | |
| 873 | Homo sapiens beculoviral IAP repeat-containing 6 (apollon) (BIRC6), mRNA | NM_016252 | 61744455 | BIRC6 |
| 874 | Homo sapiens stabilin 1 (STAB1), mRNA | NM_015136 | 61743977 | STAB1 |
| 875 | Homo sapiens AHNAK nucleoprotein (desmoyokin) (AHNAK), transcript variant 1, | NM_001620 | 61743953 | AHNAK |
| | mRNA | | | |
| 876 | Homo sapiens antigen MMSA-10 mRNA sequence | AY954500 | 61676856 | |
| 877 | Homo sapiens membrane component, chromosome 11, surface marker 1 | NM_005898 | 61676201 | M11S1 |
| | (M11S1), transcript variant 1, mRNA | | | |
| 878 | Homo sapiens prostaglandin I2 (prostacyclin) synthase (PTGIS), mRNA | NM_000961 | 61676177 | PTGIS |
| 879 | Homo sapiens cadherin 13, H-cadherin (heart) (CDH13), mRNA | NM_001257 | 61676095 | CDH13 |
| 880 | cr: gi\|61364612\|gb\|AY890631.1\|Synthetic construct Homo sapiens clone | AY890631 | 61364612 | MMP23B |
| | FLH119426.01X matrix metalloproteinase 23B (MMP23B) mRNA, complete cds | | | |
| 881 | Synthetic construct Homo sapiens clone FLH163776.01L hypothetical protein | AY888096 | 61354687 | FLJ10074 |
| | FLJ10074 (FLJ10074) mRNA, partial cds | | | |
| 882 | H. sapiens DNA sequence | Z22285 | 608767 | |
| 883 | Homo sapiens F-box protein FBX29 (FBX29) mRNA, partial cds | AF176707 | 6063091 | FBX29 |
| 884 | Homo sapiens granulin (GRN), transcript variant 2, mRNA | NM_001012479 | 60498994 | GRN |
| 885 | Homo sapiens elastin microfibril interfacer 2 (EMILIN2), mRNA | NM_032048 | 60498977 | EMILIN2 |
| 886 | Homo sapiens Uox pseudogene for urate oxidase | AB074326 | 60391530 | |
| 887 | Homo sapiens platelet-activating factor acetylhydrolase, isoform lb, alpha subunit | NM_000430 | 6031206 | PAFAH1B1 |
| | 45 kDa (PAFAH1B1), mRNA | | | |
| 888 | Homo sapiens coagulation factor II (thrombin) receptor (F2R), mRNA | NM_001992 | 6031164 | F2R |
| 889 | Homo sapiens U2 (RNU2) small nuclear RNA auxiliary factor 2 (U2AF2), transcript | NM_007279 | 60279266 | RNU2 |
| | variant 1, mRNA | | | |
| 890 | Homo sapiens Sec61 gamma subunit (SEC61G), transcript variant 2, mRNA | NM_001012456 | 60279264 | SEC61G |
| 891 | Homo sapiens mRNA; cDNA DKFZp686G0883 (from clone DKFZp686G0883) | CR936762 | 60219673 | |
| 892 | Homo sapiens mRNA; cDNA DKFZp686M208 (from clone DKFZp686M208) | CR936665 | 60219576 | |
| 893 | Homo sapiens potassium voltage-gated channel, lsk-related family, member 1 | NM_000219 | 60218914 | KCNE1 |
| | (KCNE1), mRNA | | | |
| 894 | Homo sapiens Sec61 alpha 1 subunit (S. cerevisiae) (SEC61A1), mRNA | NM_013336 | 60218911 | SEC61A1 |
| 895 | Homo sapiens TEA domain family member 1 (SV40 transcriptional enhancer | NM_021961 | 60115820 | TEAD1 |
| | factor) (TEAD1), mRNA | | | |
| 896 | Homo sapiens neuroblastoma RAS viral (v-ras) oncogene homolog (NRAS), | NM_002524 | 6006027 | NRAS |
| | mRNA | | | |
| 897 | Homo sapiens membrane protein, palmitoylated 1, 55 kDa (MPP1), mRNA | NM_002436 | 6006024 | MPP1 |
| 898 | Homo sapiens lectin, galactoside-binding, soluble, 3 binding protein (LGALS3BP), | NM_005567 | 6006016 | LGALS3BP |
| | mRNA | | | |
| 899 | Homo sapiens lectin, galactoside-binding, soluble, 1 (galectin 1) (LGALS1), mRNA | NM_002305 | 6006015 | LGALS1 |
| 900 | Homo sapiens glutathione peroxidase 3 (plasma) (GPX3), mRNA | NM_002084 | 6006000 | GPX3 |
| 901 | Homo sapiens clathrin, light polypeptide (Lca) (CLTA), transcript variant brain- | NM_007096 | 6005992 | CLTA |
| | specific, mRNA | | | |
| 902 | Homo sapiens PRA1 domain family, member 2 (PRAF2), mRNA | NM_007213 | 6005793 | PRAF2 |
| 903 | Homo sapiens suppressor of S. cerevisiae gcr2 (HSGT1), mRNA | NM_007265 | 6005783 | HSGT1 |
| 904 | Homo sapiens KIT ligand (KITLG), transcript variant b, mRNA | NM_000899 | 59939901 | KITLG |
| 905 | Homo sapiens Kruppel-like factor 9 (KLF9), mRNA | NM_001206 | 59853224 | KLF9 |
| 906 | Homo sapiens latrophilin 3 (LPHN3), mRNA | NM_015236 | 59814434 | LPHN3 |
| 907 | Homo sapiens dpy-30-like protein (LOC84661), mRNA | NM_032574 | 59710088 | LOC84661 |
| 908 | Homo sapiens frizzled homolog 2 (Drosophila) (FZD2), mRNA | NM_001466 | 5922012 | FZD2 |
| 909 | Homo sapiens calreticulin (CALR), mRNA | NM_004343 | 5921996 | CALR |
| 910 | Homo sapiens spectrin, beta, non-erythrocytic 2 (SPTBN2), mRNA | NM_006946 | 5902121 | SPTBN2 |
| 911 | Homo sapiens solute carrier family 2 (facilitated glucose transporter), member 3 | NM_006931 | 5902089 | SLC2A3 |
| | (SLC2A3), mRNA | | | |
| 912 | Homo sapiens ribonuclease P 14 kDa subunit (RPP14), mRNA | NM_007042 | 5902065 | RPP14 |
| 913 | Homo sapiens elastin microfibril interfacer 1 (EMILIN1), mRNA | NM_007046 | 5901943 | EMILIN1 |
| 914 | Homo sapiens yrdC domain containing (E. coli) (YRDC), mRNA | NM_024640 | 58761509 | YRDC |
| 915 | Homo sapiens midnolin (MIDN), mRNA | NM_177401 | 58761507 | MIDN |
| 916 | Homo sapiens serine/threonine kinase 11 (Peutz-Jeghers syndrome) (STK11), | NM_000455 | 58530881 | STK11 |
| | mRNA | | | |
| 917 | Homo sapiens homocysteine-inducible, endoplasmic reticulum stress-inducible, | NM_014685 | 58530855 | HERPUD1 |
| | ubiquitin-like domain member 1 (HERPUD1), transcript variant 1, mRNA | | | |
| 918 | Homo sapiens destrin (actin depolymerizing factor) (DSTN), transcript variant 1, | NM_006870 | 58530846 | DSTN |
| | mRNA | | | |
| 919 | Homo sapiens desmoplakin (DSP), transcript variant 1, mRNA | NM_004415 | 58530839 | DSP |
| 920 | Homo sapiens deoxyribonuclease I-like 1 (DNASE1L1), transcript variant 4, mRNA | NM_001009934 | 58430945 | DNASE1L1 |
| 921 | Homo sapiens abhydrolase domain containing 11 (ABHD11), transcript variant 2, | NM_148913 | 58430929 | ABHD11 |
| | mRNA | | | |
| 922 | Homo sapiens aquarius homolog (mouse) (AQR), mRNA | NM_014691 | 58374127 | AQR |
| 923 | Homo sapiens chaperonin containing TCP1, subunit 5 (epsilon) (CCT5), mRNA | NM_012073 | 58331232 | CCT5 |
| 924 | Homo sapiens chaperonin containing TCP1, subunit 7 (eta) (CCT7), transcript | NM_006429 | 58331183 | CCT7 |
| | variant 1, mRNA | | | |
| 925 | Homo sapiens chaperonin containing TCP1, subunit 6A (zeta 1) (CCT6A), | NM_001762 | 58331169 | CCT6A |
| | transcript variant 1, mRNA | | | |
| 926 | Homo sapiens mRNA for KIAA0099 protein, partial cds | D43951 | 58257645 | |
| 927 | Homo sapiens RAP1B, member of RAS oncogene family (RAP1B), transcript | NM_015646 | 58219793 | RAP1B |
| | variant 1, mRNA | | | |
| 928 | Homo sapiens stress-induced-phosphoprotein 1 (Hsp70/Hsp90-organizing protein) | NM_006819 | 5803180 | STIP1 |
| | (STIP1), mRNA | | | |
| 929 | Homo sapiens GLI pathogenesis-related 1 (glioma) (GLIPR1), mRNA | NM_006851 | 5803150 | GLIPR1 |
| 930 | Homo sapiens leukocyte immunoglobulin-like receptor, subfamily A (with TM | NM_006866 | 5803067 | LILRA2 |
| | domain), member 2 (LILRA2), mRNA | | | |
| 931 | Homo sapiens chromosome 22 open reading frame 9 (C22orf9), transcript variant | NM_015264 | 57863292 | C22orf9 |
| | 1, mRNA | | | |
| 932 | Homo sapiens KIAA0179 (KIAA0179), mRNA | NM_015056 | 57863268 | KIAA0179 |
| 933 | Homo sapiens heparan sulfate (glucosamine) 3-O-sulfotransferase 6 (HS3ST6), | NM_001009606 | 57528079 | HS3ST6 |
| | mRNA | | | |
| 934 | Homo sapiens t-complex-associated-testis-expressed 1-like 1 (TCTEL1), mRNA | NM_006519 | 5730084 | TCTEL1 |
| 935 | Homo sapiens fibrinogen-like 2 (FGL2), mRNA | NM_006682 | 5730074 | FGL2 |
| 936 | Homo sapiens solute carrier family 2 (facilitated glucose transporter), member 1 | NM_006516 | 5730050 | SLC2A1 |
| | (SLC2A1), mRNA | | | |
| 937 | Homo sapiens solute carrier family 12 (potassium/chloride transporters), member 7 | NM_006598 | 5730042 | SLC12A7 |
| | (SLC12A7), mRNA | | | |
| 938 | Homo sapiens Jumonji, AT rich interactive domain 1B (RBP2-like) (JARID1B), | NM_006618 | 57242795 | JARID1B |
| | mRNA | | | |
| 939 | Homo sapiens IQ motif containing GTPase activating protein 1 (IQGAP1), mRNA | NM_003870 | 57242794 | IQGAP1 |
| 940 | Homo sapiens KIAA0100 gene product (KIAA0100), mRNA | NM_014680 | 57242773 | KIAA0100 |
| 941 | Homo sapiens calsyntenin 1 (CLSTN1), transcript variant 2, mRNA | NM_014944 | 57242754 | CLSTN1 |
| 942 | Homo sapiens protein phosphatase 2 (formerly 2A), catalytic subunit, alpha | NM_002715 | 57222566 | PPP2CA |
| | isoform (PPP2CA), mRNA | | | |
| 943 | Homo sapiens microfibrillar-associated protein 3-like (MFAP3L), transcript variant | NM_021647 | 57222248 | MFAP3L |
| | 1, mRNA | | | |
| 944 | Homo sapiens RNA binding protein with multiple splicing (RBPMS), transcript | NM_001008710 | 57164968 | RBPMS |
| | variant 1, mRNA | | | |
| 945 | Homo sapiens mRNA for G7b protein (G7b gene, located in the class III region of | AJ245416 | 5701851 | |
| | the major histocompatibility complex | | | |
| 946 | Homo sapiens zinc finger protein 445 (ZNF445), mRNA | NM_181489 | 56797755 | ZNF445 |
| 947 | Homo sapiens THAP domain containing 7 (THAP7), transcript variant 2, mRNA | NM_001008695 | 56788350 | THAP7 |
| 948 | Homo sapiens cysteine dioxygenase, type I (CDO1), mRNA | NM_001801 | 56786146 | CDO1 |
| 949 | Homo sapiens copper metabolism (Murr1) domain containing 1 (COMMD1), | NM_152516 | 56786145 | COMMD1 |
| | mRNA | | | |
| 950 | Homo sapiens ubiquitin specific peptidase 20 (USP20), transcript variant 2, mRNA | NM_006676 | 56713257 | USP20 |
| 951 | Homo sapiens zinc finger CCCH-type containing 11A (ZC3H11A), mRNA | NM_014827 | 56711291 | ZC3H11A |
| 952 | Homo sapiens protein inhibitor of activated STAT, 2 (PIAS2), transcript variant | NM_004671 | 56699457 | PIAS2 |
| | beta, mRNA | | | |
| 953 | Homo sapiens nuclear receptor subfamily 1, group H, member 2 (NR1H2), mRNA | NM_007121 | 56699414 | NR1H2 |
| 954 | Homo sapiens legumain (LGMN), transcript variant 1, mRNA | NM_005606 | 56682961 | LGMN |
| 955 | Homo sapiens chromosome 12 open reading frame 10 (C12orf10), mRNA | NM_021640 | 56676384 | C12orf10 |
| 956 | Homo sapiens brain specific protein (CGI-38), mRNA | NM_015964 | 56676376 | CGI-38 |
| 957 | Homo sapiens calponin 1, basic, smooth muscle (CNN1), mRNA | NM_001299 | 56676373 | CNN1 |
| 958 | Homo sapiens guanylate kinase 1 (GUK1), mRNA | NM_000858 | 56676331 | GUK1 |
| 959 | Homo sapiens keratinocyte associated protein 2 (KRTCAP2), mRNA | NM_173852 | 56676325 | KRTCAP2 |
| 960 | Homo sapiens similar to CG14977-PA (LOC389541), mRNA | NM_001008395 | 56606126 | LOC389541 |
| 961 | Homo sapiens transmembrane protein 64 (TMEM64), mRNA | NM_001008495 | 56606065 | TMEM64 |
| 962 | Homo sapiens similar to RIKEN cDNA 2310016C16 (LOC493869), mRNA | NM_001008397 | 56605999 | LOC493869 |
| 963 | Homo sapiens torsin family 1, member A (torsin A) (TOR1A), mRNA | NM_000113 | 56550122 | TOR1A |
| 965 | Homo sapiens sarcolemma associated protein (SLMAP), mRNA | NM_007159 | 56550042 | SLMAP |
| 966 | Homo sapiens myeloid/lymphoid or mixed-lineage leukemia (trithorax homolog, | NM_005933 | 56550038 | MLL |
| | Drosophila) (MLL), mRNA | | | |
| 967 | Homo sapiens sterol carrier protein 2 (SCP2), transcript variant 1, mRNA | NM_002979 | 56243511 | SCP2 |
| 968 | Homo sapiens aldo-keto reductase family 1, member C1 (dihydrodiol | NM_001353 | 56121816 | AKR1C1 |
| | dehydrogenase 1; 20-alpha (3-alpha)-hydroxysteroid dehydrogenase) (AKR1C1), | | | |
| | mRNA | | | |
| 969 | Homo sapiens zinc finger protein 161 (ZNF161), mRNA | NM_007146 | 56118213 | ZNF161 |
| 970 | Homo sapiens cold shock domain containing E1, RNA-binding (CSDE1), transcript | NM_001007553 | 56117851 | CSDE1 |
| | variant 1, mRNA | | | |
| 971 | Homo sapiens cold shock domain containing E1, RNA-binding (CSDE1), transcript | NM_007158 | 56117849 | CSDE1 |
| | variant 2, mRNA | | | |
| 972 | Homo sapiens tripeptidyl peptidase I (TPP1), mRNA | NM_000391 | 5597012 | TPP1 |
| 973 | Homo sapiens bromodomain and WD repeat domain containing 1 (BRWD1), | NM_001007246 | 55956929 | BRWD1 |
| | transcript variant 3, mRNA | | | |
| 974 | Homo sapiens alpha 1,4-galactosyltransferase (globotriaosylceramide synthase) | NM_017436 | 55956926 | A4GALT |
| | (A4GALT), mRNA | | | |
| 975 | Homo sapiens heterogeneous nuclear ribonucleoprotein A/B (HNRPAB), transcript | NM_031266 | 55956918 | HNRPAB |
| | variant 1, mRNA | | | |
| 976 | Homo sapiens interferon-related developmental regulator 1 (IFRD1), transcript | NM_001550 | 55953128 | IFRD1 |
| | variant 1, mRNA | | | |
| 977 | Homo sapiens hypothetical protein HSPC016 (HSPC016), mRNA | NM_015933 | 55926211 | HSPC016 |
| 978 | Homo sapiens PHD finger protein 5A (PHF5A), mRNA | NM_032758 | 55925655 | PHF5A |
| 979 | Homo sapiens calcyclin binding protein (CACYBP), transcript variant 1, mRNA | NM_014412 | 55925645 | CACYBP |
| 980 | Homo sapiens mRNA for G16 protein (G16 gene located in the class III region of | AJ243936 | 5578772 | |
| | the major histocompatibility complex) | | | |
| 981 | Homo sapiens I(3)mbt-like 3 (Drosophila) (L3MBTL3), transcript variant 1, mRNA | NM_032438 | 55770869 | L3MBTL3 |
| 982 | Homo sapiens catenin (cadherin-associated protein), alpha 1, 102 kDa (CTNNA1), | NM_001903 | 55770843 | CTNNA1 |
| | mRNA | | | |
| 983 | Homo sapiens saccharopine dehydrogenase (putative) (SCCPDH), mRNA | NM_016002 | 55770835 | SCCPDH |
| 984 | Homo sapiens chromosome 4 open reading frame 9 (C4orf9), mRNA | NM_003703 | 55769586 | C4orf9 |
| 986 | Homo sapiens zinc finger protein 670 (ZNF670), mRNA | NM_033213 | 55769531 | ZNF670 |
| 987 | Homo sapiens desmin (DES), mRNA | NM_001927 | 55749931 | DES |
| 988 | Homo sapiens KIAA1815 (KIAA1815), mRNA | NM_024896 | 55749803 | KIAA1815 |
| 990 | Homo sapiens splicing factor 3b, subunit 2, 145 kDa (SF3B2), mRNA | NM_006842 | 55749530 | SF3B2 |
| 991 | Homo sapiens syndecan binding protein (syntenin) (SDCBP), transcript variant 3, | NM_001007068 | 55749503 | syntenin |
| | mRNA | | | |
| 992 | Homo sapiens transcription elongation factor A (SII)-like 8 (TCEAL8), transcript | NM_001006684 | 55749465 | SII |
| | variant 2, mRNA | | | |
| 993 | Homo sapiens WD repeat domain 26 (WDR26), mRNA | NM_025160 | 55743152 | WDR26 |
| 994 | Homo sapiens WW domain binding protein 5 (WBP5), transcript variant 3, mRNA | NM_001006613 | 55743144 | WBP5 |
| 995 | Homo sapiens collagen, type VI, alpha 3 (COL6A3), transcript variant 4, mRNA | NM_057166 | 55743103 | COL6A3 |
| 996 | Homo sapiens guanine nucleotide binding protein (G protein), beta polypeptide 2- | NM_006098 | 55742675 | GNB2L1 |
| | like 1 (GNB2L1), mRNA | | | |
| 997 | Homo sapiens DNA helicase homolog (PIF1) mRNA, partial cds | AF108138 | 5523989 | PIF1 |
| 998 | Homo sapiens eukaryotic translation initiation factor 4E (EIF4E), mRNA | NM_001968 | 54873625 | EIF4E |
| 999 | Homo sapiens arginine-rich, mutated in early stage tumors (ARMET), mRNA | NM_006010 | 54873599 | ARMET |
| 1000 | Homo sapiens glycine N-methyltransferase (GNMT), mRNA | NM_018960 | 54792737 | GNMT |
| 1001 | Homo sapiens serpin peptidase inhibitor, clade F (alpha-2 antiplasmin, pigment | NM_002615 | 54792142 | SERPINF1 |
| | epithelium derived factor), member 1 (SERPINF1), mRNA | | | |
| 1002 | Homo sapiens phosphoinositide-3-kinase, catalytic, alpha polypeptide (PIK3CA), | NM_006218 | 54792081 | PIK3CA |
| | mRNA | | | |
| 1003 | Homo sapiens metaxin 2 (MTX2), transcript variant 1, mRNA | NM_006554 | 54792075 | MTX2 |
| 1004 | Homo sapiens mRNA for KIAA0638 splice variant 2 | AB177859 | 54633201 | |
| 1006 | troponin T human, skeletal and cardiac muscle, mRNA, 932 nt | S69208 | 546020 | |
| 1007 | Homo sapiens X-ray repair complementing defective repair in Chinese hamster | NM_006297 | 5454171 | XRCC1 |
| | cells 1 (XRCC1), mRNA | | | |
| 1008 | Homo sapiens valyl-tRNA synthetase (VARS), mRNA | NM_006295 | 5454157 | VARS |
| 1009 | Homo sapiens signal sequence receptor, delta (translocon-associated protein | NM_006280 | 5454089 | SSR4 |
| | delta) (SSR4), mRNA | | | |
| 1010 | Homo sapiens proliferation-associated 2G4, 38 kDa (PA2G4), mRNA | NM_006191 | 5453841 | PA2G4 |
| 1011 | Homo sapiens periostin, osteoblast specific factor (POSTN), mRNA | NM_006475 | 5453833 | POSTN |
| 1012 | Homo sapiens neurofilament, light polypeptide 68 kDa (NEFL), mRNA | NM_006158 | 5453761 | NEFL |
| 1013 | Homo sapiens neural precursor cell expressed, developmentally down-regulated 8 | NM_006156 | 5453759 | NEDD8 |
| | (NEDD8), mRNA | | | |
| 1014 | Homo sapiens heat shock 27 kDa protein 3 (HSPB3), mRNA | NM_006308 | 5453687 | HSPB3 |
| 1015 | Homo sapiens APG7 autophagy 7-like (S. cerevisiae) (APG7L), mRNA | NM_006395 | 5453667 | APG7L |
| 1016 | Homo sapiens split hand/foot malformation (ectrodactyly) type 1 (SHFM1), mRNA | NM_006304 | 5453639 | SHFM1 |
| 1017 | Homo sapiens DKFZP564O0823 protein (DKFZP564O0823), mRNA | NM_015393 | 54400757 | DKFZP564O0823 |
| 1018 | Homo sapiens guanine nucleotide binding protein (G protein), gamma 2 (GNG2), | NM_053064 | 54262138 | GNG2 |
| | mRNA | | | |
| 1019 | Homo sapiens influenza virus NS1A binding protein (IVNS1ABP), transcript variant | NM_006469 | 54144638 | IVNS1ABP |
| | 1, mRNA | | | |
| 1020 | Homo sapiens F-box protein 30 (FBXO30), mRNA | NM_032145 | 54112383 | FBXO30 |
| 1021 | Homo sapiens splicing factor 3a, subunit 3, 60 kDa (SF3A3), mRNA | NM_006802 | 54112115 | SF3A3 |
| 1022 | Homo sapiens C-type lectin domain family 10, member A (CLEC10A), transcript | NM_006344 | 53832015 | CLEC10A |
| | variant 2, mRNA | | | |
| 1024 | Homo sapiens membrane-associated ring finger (C3HC4) 7 (MARCH7), mRNA | NM_022826 | 53828927 | MARCH7 |
| 1025 | Homo sapiens SPTAN1 mRNA for non-erythrocytic spectrin alpha, complete cds | AB191262 | 53791224 | |
| 1026 | Homo sapiens stearoyl-CoA desaturase (delta-9-desaturase) (SCD), mRNA | NM_005063 | 53759150 | SCD |
| 1029 | Homo sapiens argininosuccinate synthetase (ASS), transcript variant 1, mRNA | NM_000050 | 53759106 | ASS |
| 1030 | Homo sapiens mediator of RNA polymerase II transcription, subunit 28 homolog | NM_025205 | 53749660 | yeast |
| | (yeast) (MED28), mRNA | | | |
| 1031 | Homo sapiens WD repeat domain 1 (WDR1), transcript variant 1, mRNA | NM_017491 | 53729350 | WDR1 |
| 1032 | Homo sapiens nucleolar protein family A, member 2 (H/ACA small nucleolar | NM_017838 | 53729323 | NOLA2 |
| | RNPs) (NOLA2), mRNA | | | |
| 1033 | Homo sapiens ARP2 actin-related protein 2 homolog (yeast) (ACTR2), transcript | NM_005722 | 53692185 | ACTR2 |
| | variant 2, mRNA | | | |
| 1034 | Homo sapiens F-box protein 21 (FBXO21), transcript variant 2, mRNA | NM_015002 | 53692181 | FBXO21 |
| 1035 | Homo sapiens F-box protein 21 (FBXO21), transcript variant 1, mRNA | NM_033624 | 53692180 | FBXO21 |
| 1036 | Homo sapiens hypothetical protein H41 (H41), mRNA | NM_017548 | 52856418 | H41 |
| 1037 | Homo sapiens UTP11-iike, U3 small nucleolar ribonucleoprotein, (yeast) | NM_016037 | 52856412 | UTP11L |
| | (UTP11L), mRNA | | | |
| 1038 | Homo sapiens triosephosphate isomerase 1 (TPI1), mRNA | NM_000365 | 52851448 | TPI1 |
| 1039 | Homo sapiens dendritic cell protein (hfl-B5), mRNA | NM_006360 | 52851442 | hfl-B5 |
| 1040 | Homo sapiens glioma tumor suppressor candidate region gene 2 (GLTSCR2), | NM_015710 | 52851435 | GLTSCR2 |
| | mRNA | | | |
| 1041 | Homo sapiens replication protein A3, 14 kDa (RPA3), mRNA | NM_002947 | 52851430 | RPA3 |
| 1042 | Homo sapiens chromosome 1 open reading frame 119 (C1orf119), mRNA | NM_020141 | 52851428 | C1orf119 |
| 1043 | Homo sapiens transmembrane protein 30A (TMEM30A), mRNA | NM_018247 | 52694667 | TMEM30A |
| 1044 | Homo sapiens heterogeneous nuclear ribonucleoprotein L (HNRPL), transcript | NM_001533 | 52632382 | HNRPL |
| | variant 1, mRNA | | | |
| 1045 | Homo sapiens melanoma antigen famiiy D, 1 (MAGED1), transcript variant 2, | NM_006986 | 52632376 | MAGED1 |
| | mRNA | | | |
| 1046 | Homo sapiens nuclear factor, interleukin 3 regulated (NFIL3), mRNA | NM_005384 | 52630428 | NFIL3 |
| 1048 | Homo sapiens myosin, heavy polypeptlde 2, skeletal muscle, adult (MYH2), mRNA | NM_017534 | 52627137 | MYH2 |
| 1049 | Homo sapiens chromosome 6 open reading frame 62 (C6orf62), mRNA | NM_030939 | 52627134 | C6orf62 |
| 1050 | Homo sapiens thioredoxin domain containing 4 (endoplasmic reticulum) | NM_015051 | 52487190 | TXNDC4 |
| | (TXNDC4), mRNA | | | |
| 1051 | Homo sapiens major histocampatibility complex, class I, B (HLA-B), mRNA | NM_005514 | 52426771 | HLA-B |
| 1052 | Homo sapiens meteorin, glial cell differentiation regulator-iike (METRNL), mRNA | NM_001004431 | 52345386 | METRNL |
| 1053 | Homo sapiens SIN3 homolog B, transcription regulator (yeast) (SIN3B), mRNA | NM_015260 | 52138512 | SIN3B |
| 1054 | Homo sapiens adenosylmethionine decarboxylase 1 (AMD1), mRNA | NM_001634 | 5209326 | AMD1 |
| 1055 | Homo sapiens tumor suppressor candidate 1 (TUSC1), mRNA | NM_001004125 | 51944973 | TUSC1 |
| 1056 | Homo sapiens ADP-ribosylation-like factar 6 interacting protein 5 (ARL6IP5), | NM_006407 | 51944964 | ARL6IP5 |
| | mRNA | | | |
| 1057 | Homo sapiens A kinase (PRKA) anchor protein 2 (AKAP2), mRNA | NM_001004065 | 51873057 | PRKA |
| 1058 | Homo sapiens fibulin 2 (FBLN2), transcript variant 1, mRNA | NM_001004019 | 51873052 | FBLN2 |
| 1059 | Homo sapiens Cbp/p300-interacting transactivator, with Glu/Asp-rich carboxy- | NM_006079 | 51807294 | CITED2 |
| | terminal domain, 2 (CITED2), mRNA | | | |
| 1060 | Homo sapiens ubiquinol-cytochrome c reductase hinge protein (UQCRH), mRNA | NM_006004 | 5174744 | UQCRH |
| 1061 | Homo sapiens ubiquinol-cytochrome c reductase, Rieske iron-sulfur polypeptide 1 | NM_006003 | 5174742 | UQCRFS1 |
| | (UQCRFS1), mRNA | | | |
| 1064 | Homo sapiens P38IP (P38IP) mRNA, complete cds | AF093250 | 5163088 | P38IP |
| 1065 | Homo sapiens neuroligin 3 (NLGN3), mRNA | NM_018977 | 51593087 | NLGN3 |
| 1066 | Homo sapiens aminolevulinate, delta-, dehydratase (ALAD), transcript variant 2, | NM_000031 | 51558761 | ALAD |
| | mRNA | | | |
| 1068 | PREDICTED: Homo sapiens similar to SET protein (Phosphatase 2A inhibitor | XM_371672 | 51492666 | I-2PP2A |
| | I2PP2A) (I-2PP2A) (Template activating factor I) (TAF-I) (HLA-DR associated | | | |
| | protein II) (PHAPII) (Inhibitor of granzyme A-activated DNase) (IGAAD) | | | |
| | LOC389168), mRNA | | | |
| 1069 | Homo sapiens mannosidase, alpha, class 2A, member 1 (MAN2A1), mRNA | NM_002372 | 51477713 | MAN2A1 |
| 1070 | PREDICTED: Homo sapiens similar to Chain, Heat-Shock Cognate 70 kd Protein | XM_376576 | 51475878 | |
| | (44 kd Atpase N-Terminal Fragment) (E.C.3.6.1.3) Mutant With Asp 206 Replaced | | | |
| | By Ser (D206s) (LOC401308), mRNA | | | |
| 1071 | PREDICTED: Homo sapiens zinc finger protein 527 (ZNF527), mRNA | XM_030378 | 51474693 | ZNF527 |
| 1074 | PREDICTED: Homo sapiens chromosome 14 open reading frame 120 | XM_033371 | 51471746 | C14orf120 |
| | C14orf120), mRNA | | | |
| 1075 | PREDICTED: Homo sapiens KIAA0280 protein (KIAA0280), mRNA | XM_370635 | 51468833 | KIAA0280 |
| 1076 | PREDICTED: Homo sapiens pecanex-like 3 (Drosophila) (PCNXL3), mRNA | XM_374898 | 51468802 | PCNXL3 |
| 1077 | PREDICTED: Homo sapiens macrophage expressed gene 1 (MPEG1), mRNA | XM_166227 | 51468711 | MPEG1 |
| 1080 | PREDICTED: Homo sapiens chromosome 9 open reading frame 119 (C9orf119), | XM_372143 | 51467631 | C9orf119 |
| | mRNA | | | |
| 1081 | PREDICTED: Homo sapiens F-box protein 10 (FBXO10), mRNA | XM_291314 | 51467486 | FBXO10 |
| 1084 | PREDICTED: Homo sapiens chromosome 6 open reading frame 115 (C6orf115), | XM_371848 | 51465304 | C6orf115 |
| | mRNA | | | |
| 1086 | PREDICTED: Homo sapiens similar to Ac1147 (LOC442144), mRNA | XM_498026 | 51464623 | LOC442144 |
| 1087 | PREDICTED: Homo sapiens Melanoma associated gene (D2S448), mRNA | XM_056455 | 51460715 | D2S448 |
| 1089 | PREDICTED: Homo sapiens similar to PPIA protein (LOC402102), mRNA | XM_497870 | 51460464 | LOC402102 |
| 1090 | PREDICTED: Homo sapiens similar to protein of fungal metazoan origin like (11.1 kD) | XM_496352 | 51459096 | 2C514 |
| | (2C514) (LOC440574), mRNA | | | |
| 1092 | PREDICTED: Homo sapiens hypothetical LOC148915 (LOC148915), mRNA | XM_086360 | 51458337 | LOC148915 |
| 1094 | Homo sapiens lipase A, lysosomal acid, cholesterol estarase (Wolman disease) | NM_000235 | 51317398 | LIPA |
| | (LIPA), mRNA | | | |
| 1096 | Homo sapiens myosin, heavy polypeptide 9, non-muscle (MYH9), mRNA | NM_002473 | 51317362 | MYH9 |
| 1097 | Homo sapiens HGFL gene (MGC17330), mRNA | NM_052880 | 51317357 | MGC17330 |
| 1098 | Homo sapiens mannose-6-phosphate receptor binding protein 1 (M6PRBP1), | NM_005817 | 51317356 | M6PRBP1 |
| | mRNA | | | |
| 1099 | Homo sapiens MAD2L1 binding protein (MAD2L1BP), transcript variant 1, mRNA | NM_001003690 | 51243062 | MAD2L1BP |
| 1100 | Homo sapiens RAD23 homolog B (S. cerevisiae) (RAD23B), mRNA | NM_002874 | 51173731 | RAD23B |
| 1101 | Homo sapiens chromosome 9 open reading frame 88 (C9orf88), mRNA | NM_022833 | 51093862 | C9orf88 |
| 1102 | Homo sapiens ataxin 10 (ATXN10), mRNA | NM_013236 | 51093837 | ATXN10 |
| 1103 | Homo sapiens leptin receptor (LEPR), transcript variant 1, mRNA | NM_002303 | 51093382 | LEPR |
| 1104 | Homo sapiens myosin phosphatase-Rho interacting protein (M-RIP), mRNA | NM_201274 | 50980300 | M-RIP |
| 1105 | Homo sapiens myosin, light polypeptide 4, alkali; atrial, embryonic (MYL4), | NM_001002841 | 50845427 | MYL4 |
| | transcript variant 1, mRNA | | | |
| 1106 | Homo sapiens chromosome 22 open reading frame 13 (C22orf13), mRNA | NM_031444 | 50845406 | C22orf13 |
| 1107 | Homo sapiens CD3E antigen, epsilon polypeptide (TiT3 complex) (CD3E), mRNA | NM_000733 | 50726997 | CD3E |
| 1110 | Homo sapiens vinculin (VCL), transcript variant 2, mRNA | NM_003373 | 50593538 | VCL |
| 1111 | Homo sapiens ATPase, H+ transporting, lysosomal 9 kDa, V0 subunit e | NM_003945 | 50593534 | ATP6V0E |
| | (ATP6V0E), mRNA | | | |
| 1114 | Homo sapiens HIRA interacting protein 5 (HIRIP5), transcript variant 2, mRNA | NM_001002755 | 50593020 | HIRIP5 |
| 1115 | Homo sapiens myosin, light polypeptide 7, regulatory (MYL7), mRNA | NM_021223 | 50593014 | MYL7 |
| 1116 | Homo sapiens phosphoglycerate mutase 2 (muscle) (PGAM2), mRNA | NM_000290 | 50593009 | PGAM2 |
| 1117 | full-length cDNA clone CS0DF034YK23 of Fetal brain of Homo sapiens (human) | CR626484 | 50507291 | |
| 1118 | full-length cDNA clone CS0DF010YL22 of Fetal brain of Homo sapiens (human) | CR626265 | 50507072 | |
| 1119 | full-length cDNA clone CS0DI036YF21 of Placenta Cot 25-normalized of Homo | CR625487 | 50506294 | |
| | sapiens (human) | | | |
| 1120 | full-length cDNA clone CL0BB019ZH12 of Neuroblastoma of Homo sapiens | CR624410 | 50505217 | |
| | (human) | | | |
| 1121 | full-length cDNA clone CS0DB002YC13 of Neuroblastoma Cot 10-normalized of | CR624013 | 50504820 | |
| | Homo sapiens (human) | | | |
| 1122 | full-length cDNA clone CS0DI019YM04 of Placenta Cot 25-normalized of Homo | CR622515 | 50503322 | |
| | sapiens (human) | | | |
| 1123 | full-length cDNA clone CS0DF022YL07 of Fetal brain of Homo sapiens (human) | CR622393 | 50503200 | |
| 1124 | full-length cDNA clone CS0DJ006YC05 of T cells (Jurkat cell line) Cot 10- | CR622342 | 50503149 | |
| | normalized of Homo sapiens (human) | | | |
| 1125 | full-length cDNA clone CS0DI044YP06 of Placenta Cot 25-normalized of Homo | CR622133 | 50502940 | |
| | sapiens (human) | | | |
| 1126 | full-length cDNA clone CS0DJ009YD01 of T cells (Jurkat cell line) Cot 10- | CR622099 | 50502906 | |
| | normalized of Homo sapiens (human) | | | |
| 1127 | full-length cDNA clone CS0DB006YN23 of Neuroblastoma Cot 10-normalized of | CR621588 | 50502395 | |
| | Homo sapiens (human) | | | |
| 1128 | full-length cDNA clone CS0DL004YL07 of B cells (Ramos cell line) Cot 25- | CR621024 | 50501831 | |
| | normalized of Homo sapiens (human) | | | |
| 1129 | full-length cDNA clone CS0DC003YK02 of Neuroblastoma Cot 25-normalized of | CR620376 | 50501183 | |
| | Homo sapiens (human) | | | |
| 1130 | full-length cDNA clone CS0DL011YM05 of B cells (Ramos cell line) Cot 25- | CR620309 | 50501116 | |
| | normalized of Homo sapiens (human) | | | |
| 1131 | full-length cDNA clone CS0DI056YJ24 of Placenta Cot 25-normalized of Homo | CR620068 | 50500875 | |
| | sapiens (human) | | | |
| 1132 | full-length cDNA clone CS0DK012YC18 of HeLa cells Cot 25-normalized of Homo | CR618546 | 50499353 | |
| | sapiens (human) | | | |
| 1133 | full-length cDNA clone CS0DF011YP12 of Fetal brain of Homo sapiens (human) | CR617323 | 50498130 | |
| 1134 | full-length cDNA clone CS0DC023YC13 of Neuroblastoma Cot 25-normalized of | CR616432 | 50497239 | |
| | Homo sapiens (human) | | | |
| 1135 | full-length cDNA clone CS0DE003YE04 of Placenta of Homo sapiens (human) | CR616142 | 50496949 | |
| 1136 | full-length cDNA clone CS0DC004YN01 of Neuroblastoma Cot 25-normalized of | CR616019 | 50496826 | |
| | Homo sapiens (human) | | | |
| 1137 | full-length cDNA clone CS0DI077YN15 of Placenta Cot 25-normalized of Homo | CR615454 | 50496261 | |
| | sapiens (human) | | | |
| 1138 | full-length cDNA clone CS0DC017YN21 of Neuroblastoma Cot 25-normalized of | CR615412 | 50496219 | |
| | Homo sapiens (human) | | | |
| 1139 | full-length cDNA clone CS0DC007YC01 of Neuroblastoma Cot 25-normalized of | CR615042 | 50495849 | |
| | Homo sapiens (human) | | | |
| 1140 | full-length cDNA clone CS0DB008YA09 of Neuroblastoma Cot 10-normalized of | CR614918 | 50495725 | |
| | Homo sapiens (human) | | | |
| 1141 | full-length cDNA clone CS0DJ001YD15 of T cells (Jurkat cell line) Cot 10- | CR613910 | 50494717 | |
| | normalized of Homo sapiens (human) | | | |
| 1142 | full-length cDNA clone CS0DE002YP03 of Placenta of Homo sapiens (human) | CR612956 | 50493763 | |
| 1143 | full-length cDNA clone CS0DF008YL18 of Fetal brain of Homo sapiens (human) | CR612679 | 50493486 | |
| 1144 | full-length cDNA clone CS0DB004YO23 of Neuroblastoma Cot 10-normalized of | CR611276 | 50492083 | |
| | Homo sapiens (human) | | | |
| 1145 | full-length cDNA clone CS0DI019YI14 of Placenta Cot 25-normalized of Homo | CR610107 | 50490914 | |
| | sapiens (human) | | | |
| 1146 | full-length cDNA clone CS0DI060YJ19 of Placenta Cot 25-normalized of Homo | CR609578 | 50490385 | |
| | sapians (human) | | | |
| 1147 | full-length cDNA clone CS0CAP007YI09 of Thymus of Homo sapiens (human) | CR609170 | 50489977 | |
| 1148 | full-length cDNA clone CS0DI059YF19 of Placenta Cot 25-normalized of Homo | CR609049 | 50489856 | |
| | sapiens (human) | | | |
| 1149 | full-length cDNA clone CS0DI016YM08 of Placenta Cot 25-normalized of Homo | CR608777 | 50489584 | |
| | sapiens (human) | | | |
| 1150 | full-length cDNA clone CS0DF028YO07 of Fetal brain of Homo sapiens (human) | CR608592 | 50489399 | |
| 1151 | full-length cDNA clone CS0DJ015YL04 of T cells (Jurkat cell line) Cot 10- | CR608298 | 50489105 | |
| | normalized of Homo sapiens (human) | | | |
| 1152 | full-length cDNA clone CS0DI034YG17 of Placenta Cot 25-normalized of Homo | CR608138 | 50488945 | |
| | sapiens (human) | | | |
| 1153 | full-length cDNA clone CS0DI031YE19 of Placenta Cot 25-normalized of Homo | CR607554 | 50488361 | |
| | sapiens (human) | | | |
| 1154 | full-length cDNA clone CS0DB001YA17 of Neuroblastoma Cot 10-normalized of | CR607507 | 50488314 | |
| | Homo sapiens (human) | | | |
| 1155 | full-length cDNA clone CS0DL011YN12 of B cells (Ramos cell line) Cot 25- | CR607172 | 50487979 | |
| | normalized of Homo sapiens (human) | | | |
| 1156 | full-length cDNA clone CS0DF018YK19 of Fetal brain of Homo sapiens (human) | CR608586 | 50487393 | |
| 1157 | full-length cDNA clone CS0DF032YB12 of Fetal brain of Homo sapiens (human) | CR605933 | 50486740 | |
| 1158 | full-length cDNA clone CS0DI027YE21 of Placenta Cot 25-normalized of Homo | CR604761 | 50485568 | |
| | sapiens (human) | | | |
| 1159 | full-length cDNA clone CS0DF018YJ11 of Fetal brain of Homo sapiens (human) | CR604753 | 50485560 | |
| 1160 | full-length cDNA clone CS0DI084YO10 of Placenta Cot 25-normalized of Homo | CR604743 | 50485550 | |
| | sapiens (human) | | | |
| 1161 | full-length cDNA clone CS0DA010YE20 of Neuroblastoma of Homo sapiens | CR603690 | 50484497 | |
| | (human) | | | |
| 1162 | full-length cDNA clone CS0DC017YA10 of Neuroblastoma Cot 25-normalized of | CR603029 | 50483836 | |
| | Homo sapiens (human) | | | |
| 1163 | full-length cDNA clone CS0DJ005YD11 of T cells (Jurkat cell line) Cot 10- | CR603000 | 50483807 | |
| | normalized of Homo sapiens (human) | | | |
| 1164 | full-length cDNA clone CS0DL006YG09 of B cells (Ramos cell line) Cot 25- | CR602962 | 50483769 | |
| | normalized of Homo sapiens (human) | | | |
| 1165 | full-length cDNA clone CS0DF006YB20 of Fetal brain of Homo sapiens (human) | CR602284 | 50483091 | |
| 1166 | full-length cDNA clone CS0DE006YB21 of Placenta of Homo sapiens (human) | CR601993 | 50482800 | |
| 1167 | full-length cDNA clone CS0DI057YM10 of Placenta Cot 25-normalized of Homo | CR601242 | 50482049 | |
| | sapiens (human) | | | |
| 1168 | full-length cDNA clone CS0DI053YB05 of Placenta Cot 25-normalized of Homo | CR600158 | 50480965 | |
| | sapiens (human) | | | |
| 1169 | full-length cDNA clone CS0DM011YK19 of Fetal liver of Homo sapiens (human) | CR599900 | 50480707 | |
| 1170 | full-length cDNA clone CS0DI084YG07 of Placenta Cot 25-normalized of Homo | CR597812 | 50478619 | |
| | sapiens (human) | | | |
| 1171 | full-length cDNA clone CL0BA005ZG04 of Placenta of Homo sapiens (human) | CR596943 | 50477750 | |
| 1172 | full-length cDNA clone CS0DI022YG21 of Placenta Cot 25-normalized of Homo | CR596741 | 50477548 | |
| | sapiens (human) | | | |
| 1173 | full-length cDNA clone CS0DI066YB16 of Placenta Cot 25-normalized of Homo | CR596478 | 50477285 | |
| | sapiens (human) | | | |
| 1174 | full-length cDNA clone CS0DH004YL21 of T cells (Jurkat cell line) of Homo | CR596189 | 50476996 | |
| | sapiens (human) | | | |
| 1175 | full-length cDNA clone CS0DD007YL12 of Neuroblastoma Cot 50-normalized of | CR596149 | 50476956 | |
| | Homo sapiens (human) | | | |
| 1176 | full-length cDNA clone CS0DI044YL08 of Placenta Cot 25-normalized of Homo | CR595992 | 50476799 | |
| | sapiens (human) | | | |
| 1177 | full-length cDNA clone CS0DF005YM18 of Fetal brain of Homo sapiens (human) | CR595805 | 50476612 | |
| 1178 | full-length cDNA clone CS0DK001YM17 of HeLa cells Cot 25-normalized of Homo | CR595330 | 50476137 | |
| | sapiens (human) | | | |
| 1179 | full-length cDNA clone CS0DF005YK21 of Fetal brain of Homo sapiens (human) | CR595206 | 50476013 | |
| 1180 | full-length cDNA clone CS0DI045YB05 of Placenta Cot 25-normalized of Homo | CR594717 | 50475524 | |
| | sapiens (human) | | | |
| 1181 | full-length cDNA clone CS0DI011YP01 of Placenta Cot 25-normalized of Homo | CR593247 | 50474054 | |
| | sapiens (human) | | | |
| 1182 | full-length cDNA clone CS0DI031YC12 of Placenta Cot 25-normalized of Homo | CR593139 | 50473946 | |
| | sapiens (human) | | | |
| 1183 | full-length cDNA clone CS0DF037YF09 of Fetal brain of Homo sapiens (human) | CR592602 | 50473409 | |
| 1184 | full-length cDNA clone CS0DC026YC15 of Neuroblastoma Cot 25-normalized of | CR592099 | 50472906 | |
| | Homo sapiens (human) | | | |
| 1185 | full-length cDNA clone CS0DI076YG17 of Placenta Cot 25-normalized of Homo | CR592085 | 50472892 | |
| | sapiens (human) | | | |
| 1186 | full-length cDNA clone CS0DI085YB07 of Placenta Cot 25-normalized of Homo | CR591879 | 50472686 | |
| | sapiens (human) | | | |
| 1187 | full-length cDNA clone CS0DI063YH06 of Placenta Cot 25-normalized of Homo | CR591732 | 50472539 | |
| | sapiens (human) | | | |
| 1188 | full-length cDNA clone CS0DI086YE21 of Placenta Cot 25-normalized of Homo | CR591288 | 50472095 | |
| | sapiens (human) | | | |
| 1189 | full-length cDNA clone CS0DI002YD21 of Placenta Cot 25-normalized of Homo | CR590182 | 50470989 | |
| | sapiens (human) | | | |
| 1190 | full-length cDNA clone CS0CAP003YE18 of Thymus of Homo sapiens (human) | CR590128 | 50470783 | |
| 1191 | Homo sapiens chromosome 9 open reading frame 58 (C9orf58), transcript variant | NM_031426 | 50428929 | C9orf58 |
| | 1, mRNA | | | |
| 1192 | Homo sapiens APC11 anaphase promoting complex subunit 11 homolog (yeast) | NM_016476 | 50409832 | ANAPC11 |
| | (ANAPC11), transcript variant 2, mRNA | | | |
| 1193 | Homo sapiens phosphofructokinase, liver (PFKL), transcript variant 2, mRNA | NM_002626 | 50346003 | PFKL |
| 1197 | Homo sapiens hematological and neurological expressed 1 (HN1), transcript | NM_001002033 | 50345291 | HN1 |
| | variant 3, mRNA | | | |
| 1198 | Homo sapiens DNA-damage-inducible transcript 3 (DDIT3), mRNA | NM_004083 | 50345277 | DDIT3 |
| 1199 | Homo sapiens solute carrier family 6 (neurotransmitter transporter, creatine), | NM_005629 | 5032096 | SLC6A8 |
| | member 8 (SLC6A8), mRNA | | | |
| 1200 | Homo sapiens S100 calcium binding protein A11 (calgizzarin) (S100A11), mRNA | NM_005620 | 5032056 | S100A11 |
| 1201 | Homo sapiens retinoblastoma binding protein 4 (RBBP4), mRNA | NM_005610 | 5032026 | RBBP4 |
| 1202 | Homo sapiens J-type co-chaperone HSC20 (HSC20), mRNA | NM_172002 | 50301233 | HSC20 |
| 1203 | Homo sapiens eukaryotic translation Initiation factor 4B (EIF4B), mRNA | NM_001417 | 50053794 | EIF4B |
| 1204 | Homo sapiens glycine cleavage system protein H (aminomethyl carrier) (GCSH), | NM_004483 | 49574536 | GCSH |
| | mRNA | | | |
| 1205 | Homo sapiens Kruppel-like factor 2 (lung) (KLF2), mRNA | NM_016270 | 49574523 | KLF2 |
| 1206 | Homo sapiens ATPase, Na+/K+ transporting, beta 2 polypeptide (ATP1B2), mRNA | NM_001678 | 49574490 | ATP1B2 |
| 1207 | Homo sapiens ATPase, Na+/K+ transporting, beta 1 polypeptide (ATP1B1), | NM_001677 | 49574487 | ATP1B1 |
| | transcript variant 1, mRNA | | | |
| 1208 | Homo sapiens cofilin 1 (non-muscle) (CFL1), mRNA | NM_005507 | 49472823 | CFL1 |
| 1209 | Homo sapiens Fanconi anemia, complementation group L (FANCL), mRNA | NM_018062 | 49472818 | FANCL |
| 1210 | Homo sapiens fascin homolog 1, actin-bundling protein (Strongylocentrotus | NM_003088 | 49472815 | FSCN1 |
| | purpuratus) (FSCN1), mRNA | | | |
| 1211 | Homo sapiens chromodomain helicase DNA binding protein 9 (CHD9), mRNA | NM_025134 | 49355789 | CHD9 |
| 1212 | Homo sapiens DNA fragmentation factor DFF35 (DFF35) mRNA, complete cds | AF087573 | 4926917 | DFF35 |
| 1213 | Homo sapiens syntaxin 16, mRNA (cDNA clone MGC: 90328 IMAGE: 4371823), | BC073876 | 49256596 | |
| | complete cds | | | |
| 1214 | Homo sapiens upregulated during skeletal muscle growth 5, mRNA (cDNA clone | BC072683 | 49116143 | |
| | MGC: 87882 IMAGE: 6056764), complete cds | | | |
| 1215 | Homo sapiens cyclin D binding myb-like transcription factor 1 (DMTF1), mRNA | NM_021145 | 48976050 | DMTF1 |
| 1217 | Homo sapiens SMT3 suppressor of mif two 3 homolog 3 (yeast) (SUMO3), mRNA | NM_006936 | 48928057 | SUMO3 |
| 1218 | Homo sapiens SUMO-1 activating enzyme subunit 1 (SAE1), mRNA | NM_005500 | 4885584 | SAE1 |
| 1219 | Homo sapiens myeloid leukemia factor 2 (MLF2), mRNA | NM_005439 | 4885486 | MLF2 |
| 1220 | Homo sapiens L-3-hydroxyacyl-Coenzyme A dehydrogenase, short chain | NM_005327 | 4885386 | HADHSC |
| | (HADHSC), mRNA | | | |
| 1221 | Homo sapiens glutamic-pyruvate transaminase (alanine aminotransferase) (GPT), | NM_005309 | 4885350 | GPT |
| | mRNA | | | |
| 1222 | Homo sapiens glutamate dehydrogenase 1 (GLUD1), mRNA | NM_005271 | 4885280 | GLUD1 |
| 1223 | Homo sapiens superoxide dismutase 1, soluble (amyotrophic lateral sclerosis 1 | NM_000454 | 48762945 | SOD1 |
| | (adult)) (SOD1), mRNA | | | |
| 1224 | Homo sapiens growth factor receptor-bound protein 10 (GRB10), transcript variant | NM_001001555 | 48762696 | GRB10 |
| | 4, mRNA | | | |
| 1225 | Homo sapiens mitogen-activated protein kinase kinase kinase 15 (MAP3K15), | NM_001001671 | 48717279 | MAP3K15 |
| | mRNA | | | |
| 1226 | Homo sapiens chromosome 6 open reading frame 85 (C6orf85), mRNA | NM_021945 | 48526515 | C6orf85 |
| 1228 | Homo sapiens chloride intracellular channel 1 (CLIC1), mRNA | NM_001288 | 48375182 | CLIC1 |
| 1229 | Homo sapiens U2(RNU2) small nuclear RNA auxiliary factor 1-like 2 (U2AF1L2), | NM_005089 | 4827045 | RNU2 |
| | mRNA | | | |
| 1230 | Homo sapiens splicing factor proline/glutamine-rich (polypyrimidine tract binding | NM_005066 | 4826997 | SFPQ |
| | protein associated) (SFPQ), mRNA | | | |
| 1231 | Homo sapiens glutaminyl-tRNA synthetase (QARS), mRNA | NM_005051 | 4826959 | QARS |
| 1232 | Homo sapiens ATP-binding cassette, sub-family D (ALD), member 4 (ABCD4), | NM_005050 | 4826957 | ALD |
| | transcript variant 1, mRNA | | | |
| 1233 | Homo sapiens oxidase (cytochrome c) assembly 1-like (OXA1L), mRNA | NM_005015 | 4826879 | OXA1L |
| 1234 | Homo sapiens matrix metalloproteinase 9 (gelatinase B, 92 kDa gelatinase, 92 kDa | NM_004994 | 4826835 | MMP9 |
| | type IV collagenase) (MMP9), mRNA | | | |
| 1235 | Homo sapiens capping protein (actin filament) muscle Z-line, beta (CAPZB), | NM_004930 | 4826658 | CAPZB |
| | mRNA | | | |
| 1236 | Homo sapiens high-mobility group nucleosome binding domain 1 (HMGN1), mRNA | NM_004965 | 48255932 | HMGN1 |
| 1238 | Homo sapiens transgelin (TAGLN), transcript variant 1, mRNA | NM_001001522 | 48255906 | TAGLN |
| 1239 | Homo sapiens troponin T2, cardiac (TNNT2), transcript variant 4, mRNA | NM_001001432 | 48255882 | TNNT2 |
| 1240 | Homo sapiens anaphase promoting complex subunit 1 (ANAPC1), mRNA | NM_022662 | 48093065 | ANAPC1 |
| 1241 | Homo sapiens annexin A5 (ANXA5), mRNA | NM_001154 | 4809273 | ANXA5 |
| 1242 | Homo sapiens cDNA clone MGC: 88735 IMAGE: 6263100, complete cds | BC072022 | 47938399 | |
| 1243 | Homo sapiens HMG-box transcription factor 1 (HBP1), mRNA | NM_012257 | 47834345 | HBP1 |
| 1244 | Homo sapiens Intersectin 1 (SH3 domain protein) (ITSN1), transcript variant 1, | NM_003024 | 47717122 | ITSN1 |
| | mRNA | | | |
| 1245 | Homo sapiens zinc finger, FYVE domain containing 9 (ZFYVE9), transcript variant | NM_004799 | 47717105 | ZFYVE9 |
| | 3, mRNA | | | |
| 1247 | Homo sapiens CSF2RB full length open reading frame (ORF) cDNA clone (cDNA | CR456428 | 47678386 | ORF |
| | clone C22ORF: pGEM.CSF2RB.V2) | | | |
| 1248 | Homo sapiens tubulin-specific chaperone a (TBCA), mRNA | NM_004607 | 4759211 | TBCA |
| 1249 | Homo sapiens mannose-P-dolichol utilization defect 1 (MPDU1), mRNA | NM_004870 | 4759109 | MPDU1 |
| 1250 | Homo sapiens splicing factor, arginine/serine-rich 10 (transformer 2 homolog, | NM_004593 | 4759097 | SFRS10 |
| | Drosophila) (SFRS10), mRNA | | | |
| 1252 | Homo sapiens eukaryotic translation termination factor 1 (ETF1), mRNA | NM_004730 | 4759033 | ETF1 |
| 1253 | Homo sapiens poly(A) binding protein, nuclear 1 (PABPN1), mRNA | NM_004643 | 4758875 | PABPN1 |
| 1254 | Homo sapiens kinesin family member 5B (KIF5B), mRNA | NM_004521 | 4758647 | KIF5B |
| 1255 | Homo sapiens hepatoma-derived growth factor (high-mobility group protein 1-like) | NM_004494 | 4758515 | HDGF |
| | (HDGF), mRNA | | | |
| 1256 | Homo sapiens heart and neural crest derivatives expressed 1 (HAND1), mRNA | NM_004821 | 4758505 | HAND1 |
| 1257 | Homo sapiens death-associated protein (DAP), mRNA | NM_004394 | 4758119 | DAP |
| 1258 | Homo sapiens cysteine and glycine-rich protein 1 (CSRP1), mRNA | NM_004078 | 4758085 | CSRP1 |
| 1259 | Homo sapiens chimerin (chimaerin) 2 (CHN2), mRNA | NM_004067 | 4757979 | CHN2 |
| 1260 | Homo sapiens sperm associated antigen 7 (SPAG7), mRNA | NM_004890 | 4757715 | SPAG7 |
| 1261 | Homo sapiens gap junction protein, alpha 1, 43 kDa (connexin 43) (GJA1), mRNA | NM_000165 | 4755136 | GJA1 |
| 1262 | Homo sapiens tropomyosin 2 (beta) (TPM2), transcript variant 2, mRNA | NM_213674 | 47519615 | TPM2 |
| 1263 | Homo sapiens tropomyosin 2 (beta) (TPM2), transcript variant 1, mRNA | NM_003289 | 47519592 | TPM2 |
| 1264 | Homo sapiens chromosome 22 open reading frame 16 (C22orf16), mRNA | NM_213720 | 47497975 | C22orf16 |
| 1266 | Homo sapiens carbamoyl-phosphate synthetase 2, aspartate transcarbamylase, | NM_004341 | 47458828 | CAD |
| | and dihydroorotase (CAD), mRNA | | | |
| 1267 | Homo sapiens decorin (DCN), transcript variant A2, mRNA | NM_133503 | 47419926 | DCN |
| 1268 | Homo sapiens decorin (DCN), transcript variant A1, mRNA | NM_001920 | 47419925 | DCN |
| 1269 | Homo sapiens zinc finger protein 574 (ZNF574), mRNA | NM_022752 | 47271486 | ZNF574 |
| 1270 | Homo sapiens exosome component 5 (EXOSC5), mRNA | NM_020158 | 47174863 | EXOSC5 |
| 1271 | Homo sapiens protein kinase C, delta (PRKCD), transcript variant 2, mRNA | NM_212539 | 47157324 | PRKCD |
| 1272 | Homo sapiens cyclin-dependent kinase inhibitor 2B (p15, inhibits CDK4) | NM_004936 | 47132608 | CDKN2B |
| | (CDKN2B), transcript variant 1, mRNA | | | |
| 1274 | Homo sapiens protein kinase, cAMP-dependent, regulatory, type I, alpha (tissue | NM_002734 | 47132579 | PRKAR1A |
| | specific extinguisher 1) (PRKAR1A), transcript variant 1, mRNA | | | |
| 1275 | Homo sapiens myosin, light polypeptide kinase (MYLK), transcript variant 2, mRNA | NM_053026 | 47132562 | MYLK |
| 1276 | Homo sapiens fibronectin 1 (FN1), transcript variant 4, mRNA | NM_212478 | 47132554 | FN1 |
| 1277 | Homo sapiens fibronectin 1 (FN1), transcript variant 5, mRNA | NM_212476 | 47132552 | FN1 |
| 1278 | Homo sapiens calponin 3, acidic (CNN3), mRNA | NM_001839 | 47080096 | CNN3 |
| 1279 | Homo sapiens apelin, AGTRL1 ligand (APLN), mRNA | NM_017413 | 47078297 | APLN |
| 1280 | Homo sapiens BCL2/adenovirus E1B 19 kDa interacting protein 3-like (BNIP3L), | NM_004331 | 47078259 | BNIP3L |
| | mRNA | | | |
| 1281 | Homo sapiens G protein pathway suppressor 1 (GPS1), transcript variant 1, mRNA | NM_212492 | 47078237 | GPS1 |
| 1282 | Homo sapiens cDNA FLJ16643 fis, clone TESTI4029528 | AK131475 | 47077464 | |
| 1283 | Homo sapiens cDNA FLJ16200 fis, clone CTONG2004000 | AK131265 | 47077017 | |
| 1284 | Homo sapiens upstream transcription factor 2, c-fos interacting (USF2), transcript | NM_003367 | 46877103 | USF2 |
| | variant 1, mRNA | | | |
| 1285 | Homo sapiens filamin A interacting protein 1 (FILIP1), mRNA | NM_015687 | 46852180 | FILIP1 |
| 1286 | Homo sapiens methionine adenosyltransferase II, alpha (MAT2A), mRNA | NM_005911 | 46852159 | MAT2A |
| 1288 | Homo sapiens actin, alpha, cardiac muscle (ACTC), mRNA | NM_005159 | 46411161 | ACTC |
| 1290 | Homo sapiens hypothetical protein LOC283377 (LOC283377), mRNA | NM_207344 | 46409323 | LOC283377 |
| 1291 | Homo sapiens myeloid-associated differentiation marker-like (MYADML), mRNA | NM_207329 | 46409299 | MYADML |
| 1292 | Homo sapiens pogo transposable element with ZNF domain (POGZ), transcript | NM_207171 | 46397393 | POGZ |
| | variant 2, mRNA | | | |
| 1293 | Homo sapiens similar to splicing factor, arginine/serine-rich 4 (FLJ11021), | NM_023012 | 46397370 | FLJ11021 |
| | transcript variant 1, mRNA | | | |
| 1294 | Homo sapiens latent transforming growth factor beta binding protein 2 (LTBP2), | NM_000428 | 46389563 | LTBP2 |
| | mRNA | | | |
| 1295 | Homo sapiens GRB2-associated binding protein 1 (GAB1), transcript variant 2, | NM_002039 | 46370072 | GAB1 |
| | mRNA | | | |
| 1296 | Homo sapiens v-myb myeloblastosis viral oncogene homolog (avian) (MYB), | NM_005375 | 46361979 | MYB |
| | mRNA | | | |
| 1297 | Homo sapiens myelin protein zero-like 1 (MPZL1), transcript variant 2, mRNA | NM_024569 | 46358425 | MPZL1 |
| 1298 | Homo sapiens myelin protein zero-like 1 (MPZL1), transcript variant 1, mRNA | NM_003953 | 46358424 | MPZL1 |
| 1299 | Homo sapiens HSPC038 protein (LOC51123), mRNA | NM_016096 | 46358346 | LOC51123 |
| 1300 | Homo sapiens pleckstrin homology-like domain, family A, member 3 (PHLDA3), | NM_012396 | 46358338 | PHLDA3 |
| | mRNA | | | |
| 1301 | Homo sapiens cDNA clone IMAGE: 5193590, partial cds | BC033744 | 46255769 | |
| 1302 | Homo sapiens thyroid hormone receptor, alpha (erythroblastic leukemia viral (v- | NM_199334 | 46255056 | THRA |
| | erb-a) oncogene homolog, avian) (THRA), transcript variant 1, mRNA | | | |
| 1303 | Homo sapiens chromosome 14 open reading frame 111 (C14orf111), mRNA | NM_015962 | 46255018 | C14orf111 |
| 1304 | Homo sapiens protein phosphatase 1, catalytic subunit, beta isoform (PPP1CB), | NM_002709 | 46249374 | PPP1CB |
| | transcript variant 1, mRNA | | | |
| 1305 | Homo sapiens mitogen-activated protein kinase kinase kinase kinase 4 (MAP4K4), | NM_004834 | 46249361 | MAP4K4 |
| | transcript variant 1, mRNA | | | |
| 1306 | Homo sapiens RAB40C, member RAS oncogene family (RAB40C), mRNA | NM_021168 | 46195788 | RAB40C |
| 1307 | Homo sapiens chromosome 6 open reading frame 106 (C6orf106), transcript | NM_024294 | 46094084 | C6orf106 |
| | variant 1, mRNA | | | |
| 1308 | Homo sapiens cleavage stimulation factor, 3NULL pre-RNA, subunit 2, 64 kDa, tau | NM_015235 | 46094083 | CSTF2T |
| | variant (CSTF2T), mRNA | | | |
| 1309 | Homo sapiens TSPY-like 4 (TSPYL4), mRNA | NM_021648 | 46094050 | TSPYL4 |
| 1310 | Homo sapiens family with sequence similarity 14, member B (FAM14B), mRNA | NM_206949 | 46048519 | FAM14B |
| 1311 | Homo sapiens Kruppel-like factor 3 (basic) (KLF3), mRNA | NM_016531 | 46048173 | KLF3 |
| 1312 | Homo sapiens mRNA for KIAA0940 protein, partial cds | AB023157 | 4589523 | |
| 1313 | Homo sapiens p21 (CDKN1A)-activated kinase 2 (PAK2), mRNA | NM_002577 | 45827795 | CDKN1A |
| 1314 | Homo sapiens cytochrome c oxidase subunit 8A (ubiquitous) (COX8A), mRNA | NM_004074 | 45827791 | COX8A |
| 1315 | Homo sapiens reticulon 1 (RTN1), transcript variant 3, mRNA | NM_206852 | 45827775 | RTN1 |
| 1316 | Homo sapiens reticulon 1 (RTN1), transcript variant 1, mRNA | NM_021136 | 45827774 | RTN1 |
| 1317 | Homo sapiens UDP glucuronosyltransferase 1 family, polypeptide A8 (UGT1A8), | NM_019076 | 45827768 | UGT1A8 |
| | mRNA | | | |
| 1318 | Homo sapiens UDP glucuronosyltransferase 1 family, polypeptide A4 (UGT1A4), | NM_007120 | 45827763 | UGT1A4 |
| | mRNA | | | |
| 1319 | Homo sapiens UDP glucuronosyltransferase 1 family, polypeptide A1 (UGT1A1), | NM_000463 | 45827762 | UGT1A1 |
| | mRNA | | | |
| 1320 | Homo sapiens vitamin K epoxide reductase complex, subunit 1 (VKORC1), | NM_024006 | 45827737 | VKORC1 |
| | transcript variant 1, mRNA | | | |
| 1321 | Homo sapiens SWI/SNF related, matrix associated, actin dependent regulator of | NM_003079 | 45827732 | SMARCE1 |
| | chromatin, subfamily e, member 1 (SMARCE1), mRNA | | | |
| 1322 | Homo sapiens quaking homolog, KH domain RNA binding (mouse) (QKI), | NM_206855 | 45827711 | QKI |
| | transcript variant 4, mRNA | | | |
| 1323 | Homo sapiens cDNA clone IMAGE: 30343220, containing frame-shift errors | BC067860 | 45709287 | |
| 1324 | Homo sapiens mortality factor 4 like 1 (MORF4L1), transcript variant 2, mRNA | NM_206839 | 45643134 | MORF4L1 |
| 1325 | Homo sapiens nuclear VCP-like (NVL), transcript variant 2, mRNA | NM_206840 | 45643124 | NVL |
| 1326 | Homo sapiens LIM domain binding 3 (LDB3), mRNA | NM_007078 | 45592958 | LDB3 |
| 1327 | Homo sapiens zinc finger, CSL-type containing 2 (ZCSL2), mRNA | NM_206831 | 45592951 | ZCSL2 |
| 1328 | Homo sapiens collagen, type IV, alpha 1 (COL4A1), mRNA | NM_001845 | 45580690 | COL4A1 |
| 1329 | Homo sapiens recombination activating gene 1 (RAG1), mRNA | NM_000448 | 4557840 | RAG1 |
| 1330 | Homo sapiens peptidase D (PEPD), mRNA | NM_000285 | 4557834 | PEPD |
| 1331 | Homo sapiens phenylalanine hydroxylase (PAH), mRNA | NM_000277 | 4557818 | PAH |
| 1332 | Homo sapiens myosin, light polypeptide 3, alkali; ventricular, skeletal, slow | NM_000258 | 4557776 | MYL3 |
| | (MYL3), mRNA | | | |
| 1334 | Homo sapiens lipoprotein lipase (LPL), mRNA | NM_000237 | 4557726 | LPL |
| 1335 | Homo sapiens keratin 17 (KRT17), mRNA | NM_000422 | 4557700 | KRT17 |
| 1336 | Homo sapiens jagged 1 (Alagille syndrome) (JAG1), mRNA | NM_000214 | 4557678 | JAG1 |
| 1338 | Homo sapiens growth arrest-specific 6 (GAS6), mRNA | NM_000820 | 4557616 | GAS6 |
| 1339 | Homo sapiens fumarylacetoacetate hydrolase (fumarylacetoacetase) (FAH), | NM_000137 | 4557586 | FAH |
| | mRNA | | | |
| 1340 | Homo sapiens erythropoietin receptor (EPOR), mRNA | NM_000121 | 4557561 | EPOR |
| 1341 | Homo sapiens cholinergic receptor, nicotinic, alpha polypeptide 1 (muscle) | NM_000079 | 4557456 | CHRNA1 |
| | (CHRNA1), mRNA | | | |
| 1342 | Homo sapiens cyclin-dependent kinase inhibitor 1C (p57, Kip2) (CDKN1C), mRNA | NM_000076 | 4557440 | CDKN1C |
| 1343 | Homo sapiens CDC20 cell division cycle 20 homolog (S. cerevisiae) (CDC20), | NM_001255 | 4557436 | CDC20 |
| | mRNA | | | |
| 1344 | Homo sapiens complement component 3 (C3), mRNA | NM_000064 | 4557384 | C3 |
| 1345 | Homo sapiens serine (or cysteine) proteinase inhibitor, clade G (C1 inhibitor), | NM_000062 | 4557378 | SERPING1 |
| | member 1, (angioedema, hereditary) (SERPING1), mRNA | | | |
| 1346 | Homo sapiens bone morphogenetic protein 2 (BMP2), mRNA | NM_001200 | 4557368 | BMP2 |
| 1347 | Homo sapiens adrenergic, beta-1-, receptor (ADRB1), mRNA | NM_000684 | 4557264 | ADRB1 |
| 1348 | Homo sapiens dihydropyrimidinase-like 3 (DPYSL3), mRNA | NM_001387 | 45505175 | DPYSL3 |
| 1349 | Homo sapiens solute carrier family 24, member 5 (SLC24A5), mRNA | NM_205850 | 45504368 | SLC24A5 |
| 1350 | Homo sapiens mRNA for v-myb myeloblastosis viral oncogene homologue (avian), | AJ606319 | 45502006 | c-myb |
| | (c-myb gene), splice variant 9Aii | | | gene |
| 1351 | Homo sapiens triple functional domain (PTPRF interacting) (TRIO), mRNA | NM_007118 | 45439358 | TRIO |
| 1352 | Homo sapiens transcription elongation factor A (SII), 1 (TCEA1), transcript variant | NM_006756 | 45439353 | SII |
| | 1, mRNA | | | |
| 1353 | Homo sapiens selenoprotein X, 1 (SEPX1), mRNA | NM_016332 | 45439350 | SEPX1 |
| 1354 | Homo sapiens selenoprotein S (SELS), transcript variant 2, mRNA | NM_018445 | 45439347 | SELS |
| 1355 | Homo sapiens peptidylprolyl isomerase C (cyclophilin C) (PPIC), mRNA | NM_000943 | 45439319 | PPIC |
| 1356 | Homo sapiens peptidylprolyl isomerase A (cyclophilin A) (PPIA), transcript variant | NM_203431 | 45439312 | PPIA |
| | 3, mRNA | | | |
| 1358 | Homo sapiens proteasome (prosome, macropain) subunit, alpha type, 2 (PSMA2), | NM_002787 | 45359861 | PSMA2 |
| | mRNA | | | |
| 1359 | Homo sapiens growth factor receptor-bound protein 2 (GRB2), transcript variant 1, | NM_002086 | 45359857 | GRB2 |
| | mRNA | | | |
| 1360 | Homo sapiens Ras-GTPase activating protein SH3 domain-binding protein 2 | NM_203504 | 45359845 | G3BP2 |
| | (G3BP2), transcript variant 3, mRNA | | | |
| 1361 | Homo sapiens Ras-GTPase activating protein SH3 domain-binding protein 2 | NM_012297 | 45359844 | G3BP2 |
| | (G3BP2), transcript variant 2, mRNA | | | |
| 1362 | Homo sapiens acylphosphatase 1, erythrocyte (common) type (ACYP1), transcript | NM_203488 | 45243546 | ACYP1 |
| | variant 2, mRNA | | | |
| 1363 | Homo sapiens unknown mRNA | AY547318 | 45239007 | |
| 1364 | Homo sapiens zinc finger protein 36, C3H type, homolog (mouse) (ZFP36), mRNA | NM_003407 | 4507960 | ZFP36 |
| 1365 | Homo sapiens voltage-dependent anion channel 1 (VDAC1), mRNA | NM_003374 | 4507878 | VDAC1 |
| 1366 | Homo sapiens tumor rejection antigen (gp96) 1 (TRA1), mRNA | NM_003299 | 4507676 | TRA1 |
| 1367 | Homo sapiens tropomyosin 4 (TPM4), mRNA | NM_003290 | 4507650 | TPM4 |
| 1368 | Homo sapiens troponin C, slow (TNNC1), mRNA | NM_003280 | 4507614 | TNNC1 |
| 1369 | Homo sapiens tankyrase, TRF1-interacting ankyrin-related ADP-ribose polymerase | NM_003747 | 4507612 | TNKS |
| | (TNKS), mRNA | | | |
| 1370 | Homo sapiens transforming growth factor, beta-induced, 68 kDa (TGFBI), mRNA | NM_000358 | 4507466 | TGFBI |
| 1371 | Homo sapiens transferrin receptor (p90, CD71) (TFRC), mRNA | NM_003234 | 4507456 | TFRC |
| 1372 | Homo sapiens testis enhanced gene transcript (BAX inhibitor 1) (TEGT), mRNA | NM_003217 | 4507432 | TEGT |
| 1373 | Homo sapiens teratocarcinoma-derived growth factor 1 (TDGF1), mRNA | NM_003212 | 4507424 | TDGF1 |
| 1374 | Homo sapiens T-cell acute lymphocytic leukemia 1 (TAL1), mRNA | NM_003189 | 4507362 | TAL1 |
| 1375 | Homo sapiens signal recognition particle 9 kDa (SRP9), mRNA | NM_003133 | 4507216 | SRP9 |
| 1376 | Homo sapiens superoxide dismutase 3, extracellular (SOD3), mRNA | NM_003102 | 4507150 | SOD3 |
| 1377 | Homo sapiens chemokine (C-C motif) ligand 4 (CCL4), mRNA | NM_002984 | 4506844 | CCL4 |
| 1378 | Homo sapiens sodium channel, voltage-gated, type IX, alpha (SCN9A), mRNA | NM_002977 | 4506812 | SCN9A |
| 1379 | Homo sapiens spermidine/spermine N1-acetyltransferase (SAT), mRNA | NM_002970 | 4506788 | SAT |
| 1380 | Homo sapiens sin3-associated polypeptide, 30 kDa (SAP30), mRNA | NM_003864 | 4506782 | SAP30 |
| 1381 | Homo sapiens S100 calcium binding protein A10 (annexin II ligand, calpactin I, | NM_002966 | 4506760 | S100A10 |
| | light polypeptide (p11)) (S100A10), mRNA | | | |
| 1383 | Homo sapiens protein tyrosine phosphatase, receptor-type, Z polypeptide 1 | NM_002851 | 4506328 | PTPRZ1 |
| | (PTPRZ1), mRNA | | | |
| 1384 | Homo sapiens protein S (alpha) (PROS1), mRNA | NM_000313 | 4506116 | PROS1 |
| 1385 | Homo sapiens chromosome 17 open reading frame 35 (C17orf35), mRNA | NM_003876 | 4505900 | C17orf35 |
| 1386 | Homo sapiens phosphorylase kinase, alpha 2 (liver) (PHKA2), mRNA | NM_000292 | 4505780 | PHKA2 |
| 1387 | Homo sapiens platelet factor 4 (chemokine (C—X—C motif) ligand 4) (PF4), mRNA | NM_002619 | 4505732 | PF4 |
| 1388 | Homo sapiens pyruvate dehydrogenase (lipoamide) alpha 1 (PDHA1), mRNA | NM_000284 | 4505684 | PDHA1 |
| 1389 | Homo sapiens ornithine decarboxylase 1 (ODC1), mRNA | NM_002539 | 4505488 | ODC1 |
| 1390 | Homo sapiens NADH dehydrogenase (ubiquinone) Fe—S protein 8, 23 kDa (NADH- | NM_002496 | 4505370 | NDUFS8 |
| | coenzyme Q reductase) (NDUFS8), mRNA | | | |
| 1391 | Homo sapiens SjogrenNULLs syndrome nuclear autoantigen 1 (SSNA1), mRNA | NM_003731 | 4505324 | SSNA1 |
| 1392 | Homo sapiens myosin, heavy polypeptide 8, skeletal muscle, perinatal (MYH8), | NM_002472 | 4505300 | MYH8 |
| | mRNA | | | |
| 1393 | Homo sapiens macrophage migration inhibitory factor (glycosylation-inhibiting | NM_002415 | 4505184 | MIF |
| | factor) (MIF), mRNA | | | |
| 1394 | Homo sapiens lysosomal-associated membrane protein 2 (LAMP2), transcript | NM_002294 | 4504956 | LAMP2 |
| | variant LAMP2A, mRNA | | | |
| 1395 | Homo sapiens interferon regulatory factor 1 (IRF1), mRNA | NM_002198 | 4504720 | IRF1 |
| 1396 | Homo sapiens insulin-like growth factor binding protein 7 (IGFBP7), mRNA | NM_001553 | 4504618 | IGFBP7 |
| 1397 | Homo sapiens insulin-like growth factor 2 receptor (IGF2R), mRNA | NM_000876 | 4504610 | IGF2R |
| 1398 | Homo sapiens hypoxanthine phosphoribosyltransferase 1 (Lesch-Nyhan | NM_000194 | 4504482 | HPRT1 |
| | syndrome) (HPRT1), mRNA | | | |
| 1399 | Homo sapiens heterogeneous nuclear ribonucleoprotein A1 (HNRPA1), transcript | NM_002136 | 4504444 | HNRPA1 |
| | variant 1, mRNA | | | |
| 1400 | Homo sapiens major histocompatibility complex, class II, DR beta 1 (HLA-DRB1), | NM_002124 | 4504410 | HLA-DRB1 |
| | mRNA | | | |
| 1401 | Homo sapiens human immunodeficiency virus type I enhancer binding protein 1 | NM_002114 | 4504388 | HIVEP1 |
| | (HIVEP1), mRNA | | | |
| 1402 | Homo sapiens mutS homolog 6 (E. coli) (MSH6), mRNA | NM_000179 | 4504190 | MSH6 |
| 1403 | Homo sapiens glutathione S-transferase theta 1 (GSTT1), mRNA | NM_000853 | 4504184 | GSTT1 |
| 1404 | Homo sapiens growth arrest-specific 1 (GAS1), mRNA | NM_002048 | 4503918 | GAS1 |
| 1405 | Homo sapiens flavin containing monooxygenase 5 (FMO5), mRNA | NM_001461 | 4503760 | FMO5 |
| 1406 | Homo sapiens flavin containing monooxygenase 1 (FMO1), mRNA | NM_002021 | 4503754 | FMO1 |
| 1407 | Homo sapiens fibroblast growth factor 16 (FGF16), mRNA | NM_003868 | 4503690 | FGF16 |
| 1408 | Homo sapiens epithelial membrane protein 3 (EMP3), mRNA | NM_001425 | 4503562 | EMP3 |
| 1409 | Homo sapiens epithelial membrane protein 1 (EMP1), mRNA | NM_001423 | 4503558 | EMP1 |
| 1410 | Homo sapiens eukaryotic translation initiation factor 3, subunit 6 48 kDa (EIF3S6), | NM_001568 | 4503520 | EIF3S6 |
| | mRNA | | | |
| 1411 | Homo sapiens eukaryotic translation initiation factor 3, subunit 10 theta, | NM_003750 | 4503508 | EIF3S10 |
| | 150/170 kDa (EIF3S10), mRNA | | | |
| 1412 | Homo sapiens eukaryotic translation initiation factor 2B, subunit 1 alpha, 26 kDa | NM_001414 | 4503502 | EIF2B1 |
| | (EIF2B1), mRNA | | | |
| 1413 | Homo sapiens endothelin receptor type A (EDNRA), mRNA | NM_001957 | 4503464 | EDNRA |
| 1414 | Homo sapiens dolichyl-phosphate mannosyltransferase polypeptide 1, catalytic | NM_003859 | 4503362 | DPM1 |
| | subunit (DPM1), mRNA | | | |
| 1415 | Homo sapiens defender against cell death 1 (DAD1), mRNA | NM_001344 | 4503252 | DAD1 |
| 1416 | Homo sapiens connective tissue growth factor (CTGF), mRNA | NM_001901 | 4503122 | CTGF |
| 1417 | Homo sapiens cysteine and glycine-rich protein 2 (CSRP2), mRNA | NM_001321 | 4503100 | CSRP2 |
| 1418 | Homo sapiens crystallin, alpha B (CRYAB), mRNA | NM_001885 | 4503056 | CRYAB |
| 1419 | Homo sapiens clathrin, light polypeptide (Lca) (CLTA), transcript variant nonbrain, | NM_001833 | 4502898 | CLTA |
| | mRNA | | | |
| 1420 | Homo sapiens CDC28 protein kinase regulatory subunit 1B (CKS1B), mRNA | NM_001826 | 4502856 | CKS1B |
| 1422 | Homo sapiens CD72 antigen (CD72), mRNA | NM_001782 | 4502682 | CD72 |
| 1423 | Homo sapiens B-cell translocation gene 1, anti-proliferative (BTG1), mRNA | NM_001731 | 4502472 | BTG1 |
| 1424 | Homo sapiens bone morphogenetic protein 1 (BMP1), transcript variant BMP1-1, | NM_001199 | 4502420 | BMP1 |
| | mRNA | | | |
| 1425 | Homo sapiens brain-specific angiogenesis inhibitor 2 (BAI2), mRNA | NM_001703 | 4502356 | BAI2 |
| 1427 | Homo sapiens Rho GDP dissociation inhibitor (GDI) gamma (ARHGDIG), mRNA | NM_001176 | 4502224 | GDI |
| 1428 | Homo sapiens amyloid beta (A4) precursor-like protein 2 (APLP2), mRNA | NM_001642 | 4502146 | APLP2 |
| 1429 | Homo sapiens actinin, alpha 2 (ACTN2), mRNA | NM_001103 | 4501892 | ACTN2 |
| 1430 | Homo sapiens actin, alpha 2, smooth muscle, aorta (ACTA2), mRNA | NM_001613 | 4501882 | ACTA2 |
| 1431 | Homo sapiens alanyl-tRNA synthetase (AARS), mRNA | NM_001605 | 4501840 | AARS |
| 1432 | Homo sapiens down-regulator of transcription 1, TBP-binding (negative cofactor 2) | NM_001938 | 45006969 | DR1 |
| | (DR1), mRNA | | | |
| 1433 | Homo sapiens jun B proto-oncogene (JUNB), mRNA | NM_002229 | 44921611 | JUNB |
| 1434 | Homo sapiens stathmin 1/oncoprotein 18 (STMN1), transcript variant 3, mRNA | NM_005563 | 44889961 | STMN1 |
| 1435 | Homo sapiens solute carrier family 23 (nucleobase transporters), member 1 | NM_005847 | 44680144 | SLC23A1 |
| | (SLC23A1), transcript variant 1, mRNA | | | |
| 1436 | Homo sapiens KIAA0152 (KIAA0152), mRNA | NM_014730 | 44662824 | KIAA0152 |
| 1437 | Homo sapiens similar to RIKEN cDNA 4930522D07 (LOC164153), mRNA | NM_203412 | 44662818 | LOC164153 |
| 1438 | Homo sapiens retinoblastoma binding protein 2 homolog 1 (RBBP2H1) mRNA, | AF087481 | 4322487 | RBBP2H1 |
| | complete cds | | | |
| 1439 | Homo sapiens chromosome 17 open reading frame 37 (C17orf37), mRNA | NM_032339 | 42822890 | C17orf37 |
| 1440 | Homo sapiens p21/Cdc42/Rac1-activated kinase 1 (STE20 homolog, yeast) | NM_002576 | 42794768 | PAK1 |
| | (PAK1), mRNA | | | |
| 1441 | Homo sapiens glycerol kinase (GK), transcript variant 2, mRNA | NM_000167 | 42794761 | GK |
| 1442 | Homo sapiens alpha-methylacyl-CoA racemase (AMACR), transcript variant 1, | NM_014324 | 42794624 | AMACR |
| | mRNA | | | |
| 1443 | Homo sapiens PDZ and LIM domain 7 (enigma) (PDLIM7), transcript variant 3, | NM_203353 | 42741676 | PDLIM7 |
| | mRNA | | | |
| 1444 | Homo sapiens PDZ and LIM domain 7 (enigma) (PDLIM7), transcript variant 1, | NM_005451 | 42741673 | PDLIM7 |
| | mRNA | | | |
| 1445 | Homo sapiens 15 kDa selenoprotein (SEP15), transcript variant 1, mRNA | NM_004261 | 42741647 | SEP15 |
| 1446 | Homo sapiens MTERF domain containing 2 (MTERFD2), mRNA | NM_182501 | 42740902 | MTERFD2 |
| 1447 | Homo sapiens EF-hand domain family, member D2 (EFHD2), mRNA | NM_024329 | 42734435 | EFHD2 |
| 1448 | Homo sapiens nemo like kinase (NLK), mRNA | NM_016231 | 42734431 | NLK |
| 1449 | Homo sapiens polymerase I and transcript release factor (PTRF), mRNA | NM_012232 | 42734429 | PTRF |
| 1450 | Homo sapiens dystrobrevin, alpha (DTNA), transcript variant 5, mRNA | NM_032979 | 42717996 | DTNA |
| 1451 | Homo sapiens epithelial membrane protein 2 (EMP2), mRNA | NM_001424 | 42716292 | EMP2 |
| 1452 | Homo sapiens coiled-coil-helix-coiled-coil-helix domain containing 1 (CHCHD1), | NM_203298 | 42558251 | CHCHD1 |
| | mRNA | | | |
| 1453 | Homo sapiens iroquois homeobox protein 6 (IRX6), mRNA | NM_024335 | 42544240 | IRX6 |
| 1454 | Homo sapiens NDRG family member 2 (NDRG2), transcript variant 6, mRNA | NM_201539 | 42544219 | NDRG2 |
| 1455 | Homo sapiens vesicle-associated membrane protein 3 (cellubrevin) (VAMP3), | NM_004781 | 42544205 | VAMP3 |
| | mRNA | | | |
| 1456 | Homo sapiens BMX non-receptor tyrosine kinase (BMX), mRNA | NM_001721 | 42544180 | BMX |
| 1457 | Homo sapiens forkhead box M1 (FOXM1), transcript variant 2, mRNA | NM_021953 | 42544164 | FOXM1 |
| 1459 | Homo sapiens testis expressed sequence 261 (TEX261), mRNA | NM_144582 | 42518075 | TEX261 |
| 1460 | Homo sapiens tight junction protein 2 (zona occludens 2) (TJP2), transcript variant | NM_004817 | 42518069 | TJP2 |
| | 1, mRNA | | | |
| 1461 | Homo sapiens tripartite motif-containing 41 (TRIM41), transcript variant 1, mRNA | NM_033549 | 42516571 | TRIM41 |
| 1462 | Homo sapiens voltage-dependent anion channel 2 (VDAC2), mRNA | NM_003375 | 42476280 | VDAC2 |
| 1463 | Homo sapiens alpha-NAC protein (ANAC), mRNA | NM_199290 | 42476196 | ANAC |
| 1464 | Homo sapiens 6-phosphofructo-2-kinase/fructose-2,6-biphosphatase 3 (PFKFB3), | NM_004566 | 42476167 | PFKFB3 |
| | mRNA | | | |
| 1465 | Homo sapiens protein phosphatase 1, regulatory (inhibitor) subunit 3C | NM_005398 | 42476161 | PPP1R3C |
| | (PPP1R3C), mRNA | | | |
| 1466 | Homo sapiens pleiotrophin (heparin binding growth factor 8, neurite growth- | NM_002825 | 42476152 | PTN |
| | promoting factor 1) (PTN), mRNA | | | |
| 1467 | Homo sapiens farnesyltransferase, CAAX box, beta (FNTB), mRNA | NM_002028 | 42476119 | FNTB |
| 1468 | Homo sapiens copine III (CPNE3), mRNA | NM_003909 | 41872597 | CPNE3 |
| 1469 | Homo sapiens diacylglycerol kinase, zeta 104 kDa (DGKZ), transcript variant 2, | NM_003646 | 41872506 | DGKZ |
| | mRNA | | | |
| 1470 | Human (clone L5) orphan G protein-coupled receptor mRNA, complete cds | L06797 | 414929 | |
| 1471 | Homo sapiens glutathione peroxidase 1 (GPX1), transcript variant 1, mRNA | NM_000581 | 41406083 | GPX1 |
| 1472 | Homo sapiens amyloid beta (A4) precursor protein (peptidase nexin-II, Alzheimer | NM_000484 | 41406053 | APP |
| | disease) (APP), transcript variant 1, mRNA | | | |
| 1473 | Homo sapiens reticulon 3 (RTN3), transcript variant 1, mRNA | NM_006054 | 41393611 | RTN3 |
| 1474 | Homo sapiens reticulon 3 (RTN3), transcript variant 3, mRNA | NM_201429 | 41393603 | RTN3 |
| 1475 | Homo sapiens G protein-coupled receptor kinase interactor 1 (GIT1), mRNA | NM_014030 | 41393572 | GIT1 |
| 1476 | Homo sapiens kinesin family member 1B (KIF1B), transcript variant 1, mRNA | NM_015074 | 41393562 | KIF1B |
| 1477 | Homo sapiens solute carrier family 36 (proton/amino acid symporter), member 1 | NM_078483 | 41352720 | SLC36A1 |
| | (SLC36A1), mRNA | | | |
| 1478 | Homo sapiens E3 ubiquitin protein ligase, HECT domain containing, 1 (EDD1), | NM_015902 | 41352716 | EDD1 |
| | mRNA | | | |
| 1479 | Homo sapiens lysosomal-associated protein transmembrane 4 alpha (LAPTM4A), | NM_014713 | 41352690 | LAPTM4A |
| | mRNA | | | |
| 1480 | Homo sapiens phosphofructokinase, platelet (PFKP), mRNA | NM_002627 | 41352062 | PFKP |
| 1481 | Homo sapiens transcription elongation factor A (SII), 3 (TCEA3), mRNA | NM_003196 | 41350334 | TCEA3 |
| 1482 | Homo sapiens postsynaptic protein CRIPT (CRIPT), mRNA | NM_014171 | 41350204 | CRIPT |
| 1483 | Homo sapiens RAB1A, member RAS oncogene family (RAB1A), mRNA | NM_004161 | 41350195 | RAB1A |
| 1484 | Homo sapiens SEC31-like 1 (S. cerevisiae) (SEC31L1), transcript variant 2, mRNA | NM_016211 | 41349440 | SEC31L1 |
| 1485 | Homo sapiens bone morphogenetic protein receptor, type IA (BMPR1A), mRNA | NM_004329 | 41349436 | BMPR1A |
| 1486 | Homo sapiens nuclear protein E3-3 (DKFZP564J0123), transcript variant 3, mRNA | NM_199073 | 41327784 | DKFZP564J0123 |
| 1487 | Homo sapiens DEAD (Asp-Glu-Ala-Asp) box polypeptide 48 (DDX48), mRNA | NM_014740 | 41327777 | DDX48 |
| 1488 | Homo sapiens DEAD (Asp-Glu-Ala-Asp) box polypeptide 47 (DDX47), transcript | NM_016355 | 41327774 | (DDX47 |
| | variant 1, mRNA | | | |
| 1489 | Homo sapiens aldo-keto reductase family 7, member A2 (aflatoxin aldehyde | NM_003689 | 41327763 | AKR7A2 |
| | reductase) (AKR7A2), mRNA | | | |
| 1490 | Homo sapiens epidermal growth factor receptor (erythroblastic leukemia viral (v- | NM_005228 | 41327737 | EGFR |
| | erb-b) oncogene homolog, avian) (EGFR), transcript variant 1, mRNA | | | |
| 1491 | Homo sapiens capping protein (actin filament) muscle Z-line, alpha 2 (CAPZA2), | NM_006136 | 41327721 | CAPZA2 |
| | mRNA | | | |
| 1492 | Homo sapiens chromosome 20 open reading frame 67 (C20orf67), mRNA | NM_022104 | 41327716 | C20orf67 |
| 1493 | Homo sapiens leptin receptor overlapping transcript (LEPROT), mRNA | NM_017526 | 41327153 | LEPROT |
| 1494 | Homo sapiens UDP glucuronosyltransferase 1 family, polypeptide A7 (UGT1A7), | NM_019077 | 41282212 | UGT1A7 |
| | mRNA | | | |
| 1495 | Homo sapiens glucosamine-6-phosphate deaminase 1 (GNPDA1), mRNA | NM_005471 | 41281953 | GNPDA1 |
| 1496 | Homo sapiens NIMA (never in mitosis gene a)-related kinase 11 (NEK11), | NM_145910 | 41281752 | NEK11 |
| | transcript variant 2, mRNA | | | |
| 1497 | Homo sapiens tubulin tyrosine ligase-like famliy, member 4 (TTLL4), mRNA | NM_014640 | 41281414 | TTLL4 |
| 1498 | Homo sapiens SH2 domain binding protein 1 (tetratricopeptide repeat containing) | NM_014633 | 41281407 | SH2BP1 |
| | (SH2BP1), mRNA | | | |
| 1499 | Homo sapiens soc-2 suppressor of clear homolog (C. elegans) (SHOC2), mRNA | NM_007373 | 41281397 | SHOC2 |
| 1500 | Homo sapiens farnesyl diphosphate synthase (farnesyl pyrophosphate synthetase, | NM_002004 | 41281370 | FDPS |
| | dimethylallyltranstransferase, geranyltranstransferase) (FDPS), mRNA | | | |
| 1501 | Homo sapiens serpin peptidase inhibitor, clade B (ovalbumin), member 6 | NM_004568 | 41152085 | SERPINB6 |
| 1502 | PREDICTED: Homo sapiens similar to Ubiquitin carboxyl-terminal hydrolase 7 | XM_293886 | 41147015 | LOC345576 |
| | (Ubiquitin thiolesterase 7) (Ubiquitin-specific processing protease 7) | | | |
| | (Deubiquitinating enzyme 7) (Herpesvirus associated ubiquitin-specific protease) | | | |
| | (LOC345576), mRNA | | | |
| 1503 | Homo sapiens actin filament associated protein (AFAP), transcript variant 2, | NM_198595 | 40807503 | AFAP |
| | mRNA | | | |
| 1504 | Homo sapiens acyl-CoA synthetase long-chain family member 1 (ACSL1), mRNA | NM_001995 | 40807490 | ACSL1 |
| 1505 | Homo sapiens neuralized-like 2 (Drosophila) (NEURL2), mRNA | NM_080749 | 40807486 | NEURL2 |
| 1506 | Homo sapiens p53 and DNA damage regulated 1 (PDRG1), mRNA | NM_030815 | 40807483 | PDRG1 |
| 1507 | Homo sapiens zinc finger protein 336 (ZNF336), mRNA | NM_022482 | 40807461 | ZNF336 |
| 1508 | Homo sapiens heat shock 70 kDa protein 14, mRNA (cDNA clone | BC065281 | 40807031 | |
| | IMAGE: 6172299), complete cds | | | |
| 1509 | Homo sapiens ubiquitin-conjugating enzyme E2 variant 1 (Kua-UEV), transcript | NM_003349 | 40806191 | Kua-UEV |
| | variant 2, mRNA | | | |
| 1510 | Homo sapiens cyclin G1 (CCNG1), transcript variant 2, mRNA | NM_199246 | 40805831 | CCNG1 |
| 1511 | Homo sapiens LIM and senescent cell antigen-like domains 2 (LIMS2), mRNA | NM_017980 | 40804747 | LIMS2 |
| 1512 | Homo sapiens hypothetical protein DKFZp434N062 (DKFZp434N062), mRNA | NM_199336 | 40786393 | DKFZp434N062 |
| 1513 | Homo sapiens partial mRNA for Ankyrin repeat-containing protein (ORF1) clone | AJ315766 | 40643270 | ORF1 |
| | Telethon(Italy_B31)HSPD01281 | | | |
| 1514 | Homo sapiens ADP-ribosylarginine hydrolase (ADPRH), mRNA | NM_001125 | 40549393 | ADPRH |
| 1515 | Homo sapiens BRI3 binding protein (BRI3BP), mRNA | NM_080626 | 40548423 | BRI3BP |
| 1516 | Homo sapiens DC2 protein (DC2), mRNA | NM_021227 | 40548405 | DC2 |
| 1517 | Homo sapiens deafness locus associated putative guanine nucleotide exchange | NM_012139 | 40548400 | DELGEF |
| | factor (DELGEF), mRNA | | | |
| 1518 | Homo sapiens RAB8A, member RAS oncogene family (RAB8A), mRNA | NM_005370 | 40548385 | RAB8A |
| 1519 | Homo sapiens hypothetical protein BC004337 (LOC90826), mRNA | NM_138364 | 40538771 | LOC90826 |
| 1520 | Homo sapiens aldolase B, fructose-bisphosphate (ALDOB), mRNA | NM_000035 | 40354204 | ALDOB |
| 1521 | Homo sapiens keratin 18 (KRT18), transcript variant 2, mRNA | NM_199187 | 40354194 | KRT18 |
| 1522 | Homo sapiens 3NULL(2NULL), 5NULL-bisphosphate nucleotidase 1 (BPNT1), | NM_006085 | 40353765 | 2NULL |
| | mRNA | | | |
| 1523 | Homo sapiens thrombospondin 1 (THBS1), mRNA | NM_003246 | 40317625 | THBS1 |
| 1524 | Homo sapiens transmembrane, prostate androgen induced RNA (TMEPAI), | NM_199170 | 40317617 | TMEPAI |
| | transcript variant 3, mRNA | | | |
| 1525 | Homo sapiens aminolevulinate, delta-, synthase 1 (ALAS1), transcript variant 2, | NM_199166 | 40316938 | ALAS1 |
| | mRNA | | | |
| 1526 | Homo sapiens NFKB inhibitor interacting Ras-like 2 (NKIRAS2), transcript variant | NM_017595 | 40316921 | NKIRAS2 |
| | 2, mRNA | | | |
| 1527 | Homo sapiens thrombomodulin (THBD), mRNA | NM_000361 | 40288292 | THBD |
| 1528 | Homo sapiens GATA binding protein 6 (GATA6), mRNA | NM_005257 | 40288196 | GATA6 |
| 1529 | Homo sapiens PDZ and LIM domain 2 (mystique) (PDLIM2), transcript variant 2, | NM_021630 | 40288188 | PDLIM2 |
| | mRNA | | | |
| 1530 | Homo sapiens elongation factor Tu GTP binding domain containing 1 (EFTUD1), | NM_024580 | 40255246 | EFTUD1 |
| | mRNA | | | |
| 1531 | Homo sapiens solute carrier family 39 (zinc transporter), member 13 (SLC39A13), | NM_152264 | 40255100 | SLC39A13 |
| | mRNA | | | |
| 1532 | Homo sapiens transmembrane trafficking protein (TMP21), mRNA | NM_006827 | 40255040 | TMP21 |
| 1533 | Homo sapiens sphingosine-1-phosphate phosphatase 1 (SGPP1), mRNA | NM_030791 | 40254975 | SGPP1 |
| 1535 | Homo sapiens hypothetical protein PRO1855 (PRO1855), mRNA | NM_018509 | 40254923 | PRO1855 |
| 1537 | Homo sapiens ubiquitin-like 3 (UBL3), mRNA | NM_007106 | 40254846 | UBL3 |
| 1538 | Homo sapiens heat shock 90 kDa protein 1, alpha (HSPCA), transcript variant 2, | NM_005348 | 40254815 | HSPCA |
| | mRNA | | | |
| 1539 | Homo sapiens guanine nucleotide binding protein (G protein), q polypeptide | NM_002072 | 40254461 | GNAQ |
| | (GNAQ), mRNA | | | |
| 1540 | Homo sapiens natriuretic peptide receptor A/guanylate cyclase A (atrionatriuretic | NM_000906 | 40254425 | NPR1 |
| | peptide receptor A) (NPR1), mRNA | | | |
| 1541 | Homo sapiens myosin phosphatase-Rho interacting protein, mRNA (cDNA clone | BC009982 | 40226509 | |
| | IMAGE: 4121355), partial cds | | | |
| 1542 | Homo sapiens keratin 19 (KRT19), mRNA | NM_002276 | 40217850 | KRT19 |
| 1543 | Homo sapiens phosphogluconate dehydrogenase (PGD), mRNA | NM_002631 | 40068517 | PGD |
| 1544 | Homo sapiens PTK9L protein tyrosine kinase 9-like (A6-related protein) (PTK9L), | NM_007284 | 40068460 | PTK9L |
| | mRNA | | | |
| 1545 | Homo sapiens CBF1 interacting corepressor (CIR), transcript variant 1, mRNA | NM_004882 | 40068058 | CIR |
| 1546 | Homo sapiens glutathione S-transferase M3 (brain) (GSTM3), mRNA | NM_000849 | 39995110 | GSTM3 |
| 1547 | Homo sapiens prostaglandin F receptor (FP) (PTGFR), mRNA | NM_000959 | 39995094 | PTGFR |
| 1548 | Homo sapiens NOL1/NOP2/Sun domain family, member 2 (NSUN2), mRNA | NM_017755 | 39995081 | NSUN2 |
| 1549 | Homo sapiens cyclin-dependent kinase inhibitor 2D (p19, inhibits CDK4) | NM_001800 | 39995074 | CDKN2D |
| | (CDKN2D), transcript variant 1, mRNA | | | |
| 1550 | Homo sapiens cyclin-dependent kinase inhibitor 2D (p19, inhibits CDK4) | NM_079421 | 39995073 | CDKN2D |
| | (CDKN2D), transcript variant 2, mRNA | | | |
| 1551 | Homo sapiens iroquois homeobox protein 3 (IRX3), mRNA | NM_024336 | 39930458 | IRX3 |
| 1552 | Homo sapiens tumor protein p53 inducible nuclear protein 2 (TP53INP2), mRNA | NM_021202 | 39930398 | TP53INP2 |
| 1553 | Homo sapiens proteasome (prosome, macropain) 26S subunit, non-ATPase, 8 | NM_002812 | 39812361 | PSMD8 |
| | (PSMD8), mRNA | | | |
| 1555 | Homo sapiens transglutaminase 2 (C polypeptide, protein-glutamine-gamma- | NM_004613 | 39777596 | TGM2 |
| | glutamyltransferase) (TGM2), transcript variant 1, mRNA | | | |
| 1556 | Homo sapiens polycomb group ring finger 4 (PCGF4), mRNA | NM_005180 | 39725706 | PCGF4 |
| 1557 | Homo sapiens MID1 interacting protein 1 (gastrulation specific G12-like | NM_021242 | 39725681 | MID1IP1 |
| | (zebrafish)) (MID1IP1), mRNA | | | |
| 1558 | Homo sapiens nucleobindin 1 (NUCB1), mRNA | NM_006184 | 39725676 | NUCB1 |
| 1559 | Homo sapiens CDK2-associated protein 2 (CDK2AP2), mRNA | NM_005851 | 39725675 | CDK2AP2 |
| 1560 | Homo sapiens chromosome 16 open reading frame 53 (C16orf53), mRNA | NM_024516 | 39725648 | C16orf53 |
| 1561 | Homo sapiens PDGFA associated protein 1, mRNA (cDNA clone MGC: 14445 | BC007873 | 39644786 | |
| | IMAGE: 4303959), complete cds | | | |
| 1562 | H. sapiens MSX2 mRNA for transcription factor | X69295 | 396173 | |
| 1563 | H. sapiens (D1S483) DNA segment containing (CA) repeat; clone AFM296zc9; | Z24177 | 394377 | |
| | single read | | | |
| 1564 | Homo sapiens osf-2 mRNA for osteoblast specific factor 2 (OSF-2p1), complete | D13665 | 393318 | OSF-2p1 |
| | cds | | | |
| 1565 | Homo sapiens colony stimulating factor 1 (CSF1) mRNA, partial cds | M76453 | 393119 | CSF1 |
| 1566 | H. sapiens mRNA for immunoglobulin kappa light chain VJ region (ID LAM023) | Z68974 | 3928276 | |
| 1567 | Homo sapiens mRNA for KIAA0768 protein, partial cds | AB018311 | 3882256 | |
| 1568 | Homo sapiens laminin, alpha 1 (LAMA1), mRNA | NM_005559 | 38788415 | LAMA1 |
| 1569 | Homo sapiens homolog of yeast INO80 (INO80), transcript variant 1, mRNA | NM_017553 | 38708320 | INO80 |
| 1570 | Homo sapiens ribonuclease T2 (RNASET2), mRNA | NM_003730 | 38683865 | RNASET2 |
| 1571 | Homo sapiens ankyrin repeat domain 13 (ANKRD13), mRNA | NM_033121 | 38683798 | ANKRD13 |
| 1572 | Homo sapiens acetyl-Coenzyme A carboxylase alpha (ACACA), transcript variant | NM_000664 | 38679979 | ACACA |
| | 6, mRNA | | | |
| 1573 | Homo sapiens septin 11 (SEPT11), mRNA | NM_018243 | 38605734 | SEPT11 |
| 1574 | Homo sapiens mucolipin 3 (MCOLN3), mRNA | NM_018298 | 38570136 | MCOLN3 |
| 1575 | Homo sapiens FLJ20259 protein (FLJ20259), transcript variant 1, mRNA | NM_017730 | 38570096 | FLJ20259 |
| 1576 | Homo sapiens CUG triplet repeat, RNA binding protein 1 (CUGBP1), transcript | NM_006560 | 38570082 | CUGBP1 |
| | variant 1, mRNA | | | |
| 1577 | Homo sapiens KIAA1244 (KIAA1244), mRNA | NM_020340 | 38569486 | KIAA1244 |
| 1578 | Homo sapiens metaxin 1 (MTX1), transcript variant 1, mRNA | NM_002455 | 38569474 | MTX1 |
| 1579 | Homo sapiens ATP citrate lyase (ACLY), transcript variant 2, mRNA | NM_198830 | 38569422 | ACLY |
| 1580 | Homo sapiens ATP citrate lyase (ACLY), transcript variant 1, mRNA | NM_001096 | 38569420 | ACLY |
| 1581 | Homo sapiens ADP-ribosylation factor-like 3 (ARL3), mRNA | NM_004311 | 38569402 | ARL3 |
| 1582 | Homo sapiens ras-related C3 botulinum toxin substrate 1 (rho family, small GTP | NM_006908 | 38505163 | RAC1 |
| | binding protein Rac1) (RAC1), transcript variant Rac1, mRNA | | | |
| 1583 | Homo sapiens pentatricopeptide repeat domain 1 (PTCD1), mRNA | NM_015545 | 38492357 | PTCD1 |
| 1584 | Homo sapiens arginine/serine-rich coiled-coil 1 (RSRC1), mRNA | NM_016625 | 38488726 | RSRC1 |
| 1585 | Homo sapiens cytoglobin (CYGB), mRNA | NM_134268 | 38454323 | CYGB |
| 1587 | Homo sapiens chromatin modifying protein 2A (CHMP2A), transcript variant 2, | NM_198426 | 38372932 | CHMP2A |
| | mRNA | | | |
| 1588 | Homo sapiens branched chain keto acid dehydrogenase E1, alpha polypeptide | NM_000709 | 38372929 | BCKDHA |
| | (maple syrup urine disease) (BCKDHA), mRNA | | | |
| 1589 | Homo sapiens Sp1 transcription factor (SP1), mRNA | NM_138473 | 38372900 | SP1 |
| 1590 | Homo sapiens spermatogenesis associated 21 (SPATA21), mRNA | NM_198546 | 38348381 | SPATA21 |
| 1591 | Homo sapiens chromosome 1 open reading frame 122 (C1orf122), mRNA | NM_198446 | 38348207 | C1orf122 |
| 1593 | Homo sapiens chromosome 20 open reading frame 47 (C20orf47), transcript | NM_015966 | 38327613 | C20orf47 |
| | variant 2, mRNA | | | |
| 1594 | Homo sapiens insulin induced gene 2 (INSIG2), mRNA | NM_016133 | 38327532 | INSIG2 |
| 1595 | Homo sapiens ankyrin repeat domain 1 (cardiac muscle) (ANKRD1), mRNA | NM_014391 | 38327521 | ANKRD1 |
| 1596 | Homo sapiens heat shock 70 kDa protein 4 (HSPA4), transcript variant 1, mRNA | NM_002154 | 38327038 | HSPA4 |
| 1597 | Homo sapiens ubiquitination factor E4B (UFD2 homolog, yeast) (UBE4B), mRNA | NM_006048 | 38327033 | UBE4B |
| 1598 | Homo sapiens APG12 autophagy 12-like (S. cerevisiae) (APG12L), mRNA | NM_004707 | 38261968 | APG12L |
| 1599 | Homo sapiens neural precursor cell expressed, developmentally down-regulated 4 | NM_006154 | 38257154 | NEDD4 |
| | (NEDD4), transcript variant 1, mRNA | | | |
| 1600 | Homo sapiens ephrin-B3 (EFNB3), mRNA | NM_001406 | 38201712 | EFNB3 |
| 1601 | Homo sapiens DEAD (Asp-Glu-Ala-Asp) box polypeptide 17 (DDX17), transcript | NM_030881 | 38201711 | DDX17 |
| | variant 2, mRNA | | | |
| 1602 | Homo sapiens telomerase reverse transcriptase (TERT), transcript variant 2, | NM_198255 | 38201701 | TERT |
| | mRNA | | | |
| 1603 | Homo sapiens syndecan 4 (amphiglycan, ryudocan) (SDC4), mRNA | NM_002999 | 38201674 | SDC4 |
| 1604 | Homo sapiens AYP1 protein (AYP1), mRNA | NM_032193 | 38176284 | AYP1 |
| 1605 | Homo sapiens PPAR binding protein, mRNA (cDNA clone IMAGE: 4822636), | BC060758 | 38174456 | |
| | complete cds | | | |
| 1606 | Homo sapiens splicing factor, arginine/serine-rich 6 (SFRS6), mRNA | NM_006275 | 38158029 | SFRS6 |
| 1607 | Homo sapiens solute carrier family 37 (glycerol-3-phosphate transporter), member | NM_198277 | 38093648 | SLC37A2 |
| | 2 (SLC37A2), mRNA | | | |
| 1608 | Homo sapiens ubiquitin-conjugating enzyme E2Q (putative) (UBE2Q), mRNA | NM_017582 | 38045949 | UBE2Q |
| 1609 | Homo sapiens neurotrimin (HNT), mRNA | NM_016522 | 38045920 | HNT |
| 1610 | Homo sapiens COP9 constitutive photomorphogenic homolog subunit 6 | NM_006833 | 38027945 | COPS6 |
| | (Arabidopsis) (COPS6), mRNA | | | |
| 1611 | Homo sapiens COP9 constitutive photomorphogenic homolog subunit 5 | NM_006837 | 38027922 | COPS5 |
| | (Arabidopsis) (COPS5), mRNA | | | |
| 1612 | Homo sapiens 4-hydroxyphenylpyruvate dioxygenase (HPD), mRNA | NM_002150 | 38016938 | HPD |
| 1613 | Homo sapiens prostatic binding protein (PBP), mRNA | NM_002567 | 38016928 | PBP |
| 1614 | Homo sapiens protein kinase, AMP-activated, beta 1 non-catalytic subunit | NM_006253 | 38016923 | PRKAB1 |
| | (PRKAB1), mRNA | | | |
| 1615 | Homo sapiens restin (Reed-Steinberg cell-expressed intermediate filament- | NM_002956 | 38016917 | RSN |
| | associated protein) (RSN), transcript variant 1, mRNA | | | |
| 1616 | Homo sapiens BAT2 domain containing 1 (BAT2D1), mRNA | NM_015172 | 38016902 | BAT2D1 |
| 1617 | Homo sapiens B-lymphoma and BAL associated protein (BBAP) mRNA, complete | AY225123 | 37904596 | BBAP |
| | cds | | | |
| 1618 | Homo sapiens dual specificity phosphatase 3 (vaccinia virus phosphatase VH1- | NM_004090 | 37655179 | DUSP3 |
| | related) (DUSP3), mRNA | | | |
| 1619 | Homo sapiens nudix (nucleoside diphosphate linked moiety X)-type motif 3 | NM_006703 | 37622350 | NUDT3 |
| | (NUDT3), mRNA | | | |
| 1620 | Homo sapiens PBX/knotted 1 homeobox 1 (PKNOX1), transcript variant 1, mRNA | NM_004571 | 37595549 | PKNOX1 |
| 1621 | Homo sapiens ring finger protein 128 (RNF128), transcript variant 1, mRNA | NM_194463 | 37588872 | RNF128 |
| 1622 | Homo sapiens ubiquitin-conjugating enzyme E2, J1 (UBC6 homolog, yeast) | NM_016021 | 37577121 | UBE2J1 |
| | (UBE2J1), mRNA | | | |
| 1623 | Homo sapiens antigen MLAA-37 mRNA sequence | AY288979 | 37574674 | |
| 1624 | Homo sapiens antigen MLAA-3 mRNA, partial 3′UTR sequence | AY288946 | 37574631 | |
| 1625 | Homo sapiens alanine-glyoxylate aminotransferase 2-like 1 (AGXT2L1), mRNA | NM_031279 | 37574041 | AGXT2L1 |
| 1626 | PREDICTED: Homo sapiens FLJ00133 protein (FLJ00133), mRNA | XM_059482 | 37549556 | FLJ00133 |
| 1627 | PREDICTED: Homo sapiens kinesin family member 26A (KIF26A), mRNA | XM_050278 | 37546014 | KIF26A |
| 1628 | Homo sapiens carbohydrate (chondroitin) synthase 1 (CHSY1), mRNA | NM_014918 | 37537719 | CHSY1 |
| 1629 | Homo sapiens eukaryotic translation initiation factor 5 (EIF5), transcript variant 2, | NM_183004 | 37537715 | EIF5 |
| | mRNA | | | |
| 1630 | Homo sapiens poly(A) polymerase beta (testis specific) (PAPOLB), mRNA | NM_020144 | 37202113 | PAPOLB |
| 1631 | Homo sapiens tRNA selenocysteine associated protein (SECP43), mRNA | NM_017846 | 37059777 | SECP43 |
| 1632 | Homo sapiens DEAD (Asp-Glu-Ala-Asp) box polypeptide 51 (DDX51), mRNA | NM_175066 | 37059776 | DDX51 |
| 1633 | Homo sapiens sushi domain containing 3 (SUSD3), mRNA | NM_145006 | 37059752 | SUSD3 |
| 1634 | Homo sapiens peroxisomal biogenesis factor 5 (PEX5), mRNA | NM_000319 | 37059745 | PEX5 |
| 1635 | Homo sapiens DNA segment on chromosome 4 (unique) 234 expressed sequence | NM_014392 | 36951161 | D4S234E |
| | (D4S234E), mRNA | | | |
| 1636 | Homo sapiens fukutin related protein (FKRP), mRNA | NM_024301 | 36951139 | FKRP |
| 1637 | Homo sapiens HT-1080 protein mRNA, complete cds | U27112 | 3551741 | |
| 1638 | Homo sapiens ubiquitin-conjugating enzyme E2I (UBC9 homolog, yeast) (UBE2I), | NM_003345 | 35493970 | UBE2I |
| | transcript variant 1, mRNA | | | |
| 1639 | Human mRNA for platelet-derived growth factor PDGF-A | X06374 | 35363 | |
| 1640 | Homo sapiens tripartite motif-containing 55 (TRIM55), transcript variant 1, mRNA | NM_184085 | 34878835 | TRIM55 |
| 1641 | Homo sapiens chromosome 17 open reading frame 25 (C17orf25), mRNA | NM_016080 | 34850073 | C17orf25 |
| 1643 | Homo sapiens stathmin-like 2 (STMN2), mRNA | NM_007029 | 34850060 | STMN2 |
| 1644 | Homo sapiens full length insert cDNA clone ZD17G09 | AF086233 | 3483578 | |
| 1645 | Homo sapiens frizzled homolog 6 (Drosophila) (FZD6), mRNA | NM_003506 | 34734078 | FZD6 |
| 1646 | Homo sapiens fibulin 1 (FBLN1), transcript variant D, mRNA | NM_006486 | 34734065 | FBLN1 |
| 1647 | Homo sapiens fibulin 1 (FBLN1), transcript variant C, mRNA | NM_001996 | 34734061 | FBLN1 |
| 1648 | Homo sapiens RNA-binding region (RNP1, RRM) containing 1 (RNPC1), transcript | NM_017495 | 34577106 | RNPC1 |
| | variant 1, mRNA | | | |
| 1649 | Homo sapiens RAB22A, member RAS oncogene family (RAB22A), mRNA | NM_020673 | 34577103 | RAB22A |
| 1650 | Homo sapiens retinoblastoma-like 1 (p107) (RBL1), transcript variant 2, mRNA | NM_183404 | 34577076 | RBL1 |
| 1651 | Homo sapiens adipose differentiation-related protein (ADFP), mRNA | NM_001122 | 34577058 | ADFP |
| 1652 | Homo sapiens TSC22 domain family, member 1 (TSC22D1), transcript variant 1, | NM_183422 | 34556206 | TSC22D1 |
| | mRNA | | | |
| 1653 | Homo sapiens cDNA FLJ46208 fis, clone TESTI4010979 | AK128087 | 34535285 | |
| 1654 | Homo sapiens cDNA FLJ45929 fis, clone PLACE7000266, weakly similar to Homo | AK127826 | 34534907 | TTN |
| | sapiens titin (TTN), transcript variant N2-B | | | |
| 1655 | Homo sapiens cDNA FLJ45200 fis, clone BRCAN2006051, moderately similar to | AK127143 | 34533922 | |
| | AFG3-like protein 2 (EC 3.4.24.—) | | | |
| 1656 | Homo sapiens cDNA FLJ45003 fis, clone BRAWH3011623, moderately similar to | AK126950 | 34533641 | |
| | Heterogeneous nuclear ribonucleoproteins C1/C2 | | | |
| 1657 | Homo sapiens cDNA FLJ44930 fis, clone BRAMY3015549, highly similar to L1 cell | AK126878 | 34533543 | L1CAM |
| | adhesion molecule (L1CAM) | | | |
| 1658 | Homo sapiens cDNA FLJ44370 fis, clone TRACH3008902 | AK126342 | 34532803 | |
| 1659 | Homo sapiens cDNA FLJ43892 fis, clone TESTI4009457, highly similar to Homo | AK125880 | 34532143 | TP53INP1 |
| | sapiens tumor protein p53 inducible nuclear protein 1 (TP53INP1) | | | |
| 1660 | Homo sapiens cDNA FLJ43847 fis, clone TESTI4006407 | AK125835 | 34532079 | |
| 1661 | Homo sapiens cDNA FLJ43615 fis, clone SPLEN2016480 | AK125603 | 34531749 | |
| 1662 | Homo sapiens cDNA FLJ43528 fis, clone PLACE6015211 | AK125516 | 34531637 | |
| 1663 | Homo sapiens cDNA FLJ43331 fis, clone NT2RI3006132 | AK125321 | 34531379 | |
| 1664 | Homo sapiens cDNA FLJ43306 fis, clone NT2NE2006909, highly similar to | AK125296 | 34531345 | |
| | Methionine aminopeptidase 2 (EC 3.4.11.18) | | | |
| 1665 | Homo sapiens cDNA FLJ42802 fis, clone BRCAN2002562, moderately similar to | AK124792 | 34530674 | |
| | Splicing factor, arginine/serine-rich 2 | | | |
| 1666 | Homo sapiens cDNA FLJ42775 fis, clone BRAWH3004530 | AK124765 | 34530635 | |
| 1667 | Homo sapiens cDNA FLJ41022 fis, clone ASTRO1000009, weakly similar to | AK123018 | 34528466 | |
| | BAND 4.1-LIKE PROTEIN 4 | | | |
| 1668 | Homo sapiens cDNA FLJ16566 fis, clone SYNOV4009298 | AK122900 | 34528344 | |
| 1669 | Homo sapiens ornithine decarboxylase antizyme 1 (OAZ1), mRNA | NM_004152 | 34486089 | OAZ1 |
| 1670 | Homo sapiens karyopherin alpha 3 (importin alpha 4) (KPNA3), mRNA | NM_002267 | 34485721 | KPNA3 |
| 1671 | Homo sapiens RAB11A, member RAS oncogene family (RAB11A), mRNA | NM_004663 | 34485712 | RAB11A |
| 1672 | cr: gi\|34481864\|emb\|AJ549387.1\|HSA549387 Homo sapiens mRNA for basic | AJ549387 | 34481864 | |
| | transcription factor 2 homologue (BBF2H7 gene), from lung tissue | | | |
| 1673 | Homo sapiens actinin, alpha 4 (ACTN4), mRNA | NM_004924 | 34452697 | ACTN4 |
| 1674 | Homo sapiens mRNA; cDNA DKFZp686A06190 (from clone DKFZp686A06190) | BX648808 | 34367973 | |
| 1675 | Homo sapiens mRNA; cDNA DKFZp779D1554 (from clone DKFZp779D1554) | BX648542 | 34367704 | |
| 1676 | Homo sapiens mRNA; cDNA DKFZp686C20269 (from clone DKFZp686C20269) | BX648458 | 34367620 | |
| 1677 | Homo sapiens mRNA; cDNA DKFZp686I16170 (from clone DKFZp686I16170) | BX648343 | 34367502 | |
| 1678 | Homo sapiens mRNA; cDNA DKFZp686N2252 (from clone DKFZp686N2252) | BX647990 | 34367149 | |
| 1679 | Homo sapiens mRNA; cDNA DKFZp686K02111 (from clone DKFZp686K02111) | BX647377 | 34366405 | |
| 1680 | Homo sapiens mRNA; cDNA DKFZp686O10247 (from clone DKFZp686O10247) | BX641027 | 34365404 | |
| 1681 | Homo sapiens mRNA; cDNA DKFZp686M14190 (from clone DKFZp686M14190) | BX640978 | 34365328 | |
| 1682 | Homo sapiens proteasome (prosome, macropain) 26S subunit, non-ATPase, 7 | NM_002811 | 34335279 | PSMD7 |
| | (Mov34 homolog) (PSMD7), mRNA | | | |
| 1683 | Homo sapiens popeye domain containing 3 (POPDC3), mRNA | NM_022361 | 34335275 | POPDC3 |
| 1684 | Homo sapiens prion protein (p27-30) (Creutzfeld-Jakob disease, Gerstmann- | NM_183079 | 34335269 | PRNP |
| | Strausler-Scheinker syndrome, fatal familial insomnia) (PRNP), transcript variant 2, | | | |
| | mRNA | | | |
| 1685 | Homo sapiens discs, large (Drosophila) homolog-associated protein 4 (DLGAP4), | NM_183006 | 34335250 | DLGAP4 |
| | transcript variant 2, mRNA | | | |
| 1686 | Homo sapiens creatine kinase, brain (CKB), mRNA | NM_001823 | 34335231 | CKB |
| 1687 | Homo sapiens CDC45 cell division cycle 45-like (S. cerevisiae) (CDC45L), mRNA | NM_003504 | 34335230 | CDC45L |
| 1689 | Homo sapiens BCL2-associated X protein (BAX), transcript variant alpha, mRNA | NM_138761 | 34335117 | BAX |
| 1690 | Homo sapiens thymosin, beta 4, X-linked (TMSB4X), mRNA | NM_021109 | 34328943 | TMSB4X |
| 1691 | Homo sapiens CD63 antigen (melanoma 1 antigen) (CD63), mRNA | NM_001780 | 34328936 | CD63 |
| 1692 | Homo sapiens small nuclear ribonucleoprotein D3 polypeptide 18 kDa (SNRPD3), | NM_004175 | 34328935 | SNRPD3 |
| | mRNA | | | |
| 1694 | Homo sapiens protein tyrosine phosphatase, non-receptor type 6 (PTPN6), | NM_002831 | 34328900 | PTPN6 |
| | transcript variant 1, mRNA | | | |
| 1695 | Homo sapiens tyrosine 3-monooxygenase/tryptophan 5-monooxygenase activation | NM_006761 | 34304385 | YWHAE |
| | protein, epsilon polypeptide (YWHAE), mRNA | | | |
| 1696 | Homo sapiens sorting nexin 3 (SNX3), transcript variant 1, mRNA | NM_003795 | 34304374 | SNX3 |
| 1697 | Homo sapiens follistatin-like 1 (FSTL1), mRNA | NM_007085 | 34304366 | FSTL1 |
| 1698 | Homo sapiens biglycan (BGN), mRNA | NM_001711 | 34304351 | BGN |
| 1699 | Homo sapiens integral membrane protein 1 (ITM1), mRNA | NM_152713 | 34303951 | ITM1 |
| 1700 | Homo sapiens zinc finger protein 605 (ZNF605), mRNA | NM_183238 | 34222390 | ZNF605 |
| 1701 | Homo sapiens HMP19 protein (HMP19), mRNA | NM_015980 | 34222326 | HMP19 |
| 1702 | Homo sapiens KIAA0063 gene product (KIAA0063), mRNA | NM_014876 | 34222319 | KIAA0063 |
| 1703 | Homo sapiens trafficking protein particle complex 3 (TRAPPC3), mRNA | NM_014408 | 34222308 | TRAPPC3 |
| 1704 | Homo sapiens trophoblast glycoprotein (TPBG), mRNA | NM_006670 | 34222307 | TPBG |
| 1705 | Homo sapiens cysteine-rich, angiogenic inducer, 61 (CYR61), mRNA | NM_001554 | 34222286 | CYR61 |
| 1706 | Homo sapiens tubulin, beta polypeptide (TUBB), mRNA | NM_178014 | 34222261 | TUBB |
| 1707 | Homo sapiens ARD1 homolog A, N-acetyltransferase (S. cerevisiae) (ARD1A), | NM_003491 | 34222259 | ARD1A |
| | mRNA | | | |
| 1708 | Homo sapiens claudin 7 (CLDN7), mRNA | NM_001307 | 34222214 | CLDN7 |
| 1709 | Homo sapiens hypothetical protein MGC33214 (MGC33214), mRNA | NM_153354 | 34222213 | MGC33214 |
| 1710 | Homo sapiens CD164 antigen, sialomucin (CD164), mRNA | NM_006016 | 34222157 | CD164 |
| 1711 | Homo sapiens hypothetical gene supported by AL449243 (LOC91689), mRNA | NM_033318 | 34222144 | LOC91689 |
| 1712 | Homo sapiens RAB18, member RAS oncogene family (RAB18), mRNA | NM_021252 | 34222129 | RAB18 |
| 1713 | Homo sapiens RWD domain containing 2 (RWDD2), mRNA | NM_033411 | 34222125 | RWDD2 |
| 1714 | Homo sapiens synaptotagmin IV (SYT4), mRNA | NM_020783 | 34222118 | SYT4 |
| 1715 | Homo sapiens programmed cell death 4 (neoplastic transformation inhibitor), | BC026104 | 34193739 | |
| | transcript variant 1, mRNA (cDNA clone MGC: 33046 IMAGE: 4798293), complete | | | |
| | cds | | | |
| 1716 | Homo sapiens cDNA clone IMAGE: 5263531 | BC037740 | 34191398 | |
| 1717 | Homo sapiens IQ motif containing F3, mRNA (cDNA clone IMAGE: 4828588) | BC021188 | 34190818 | |
| 1718 | Homo sapiens hypothetical protein FLJ30707, mRNA (cDNA clone | BC034497 | 34190385 | |
| | IMAGE: 4836906), complete cds | | | |
| 1719 | Homo sapiens chromosome 6 open reading frame 93 (C6orf93), mRNA | NM_032860 | 34147695 | C6orf93 |
| 1720 | Homo sapiens transforming growth factor beta 1 induced transcript 1 (TGFB1I1), | NM_015927 | 34147679 | TGFB1I1 |
| | mRNA | | | |
| 1721 | Homo sapiens four and a half LIM domains 1 (FHL1), mRNA | NM_001449 | 34147646 | FHL1 |
| 1722 | Homo sapiens hypothetical protein MGC10433 (MGC10433), mRNA | NM_024321 | 34147641 | MGC10433 |
| 1723 | Homo sapiens sorbitol dehydrogenase (SORD), mRNA | NM_003104 | 34147623 | SORD |
| 1724 | Homo sapiens Rho GDP dissociation Inhibitor (GDI) alpha (ARHGDIA), mRNA | NM_004309 | 34147601 | GDI |
| 1725 | Homo sapiens eukaryotic translation initiation factor 5A (EIF5A), mRNA | NM_001970 | 34147551 | EIF5A |
| 1726 | Homo sapiens likely ortholog of mouse gene rich cluster, C10 gene (GRCC10), | NM_138425 | 34147536 | GRCC10 |
| | mRNA | | | |
| 1727 | Homo sapiens hypothetical protein BC004507 (LOC90313), mRNA | NM_138349 | 34147526 | LOC90313 |
| 1728 | Homo sapiens COX4 neighbor (COX4NB), mRNA | NM_006067 | 34147520 | COX4NB |
| 1729 | Homo sapiens zinc finger protein 622 (ZNF622), mRNA | NM_033414 | 34147461 | ZNF622 |
| 1730 | Homo sapiens meteorin, glial cell differentiation regulator (METRN), mRNA | NM_024042 | 34147349 | METRN |
| 1731 | Homo sapiens Rho GTPase activating protein 22 (ARHGAP22), mRNA | NM_021226 | 34013589 | ARHGAP22 |
| 1732 | Human U2 small nuclear RNA-associated B″ antigen mRNA, complete cds | M15841 | 340104 | |
| 1733 | Human ubiquitin carboxyl-terminal hydrolase (PGP 9.5, UCH-L3) isozyme L3 | M30496 | 340073 | |
| | mRNA, complete cds | | | |
| 1734 | Homo sapiens cofilin 2 (muscle) (CFL2), transcript variant 1, mRNA | NM_021914 | 33946276 | CFL2 |
| 1735 | cr: gi\|33943\|emb\|X53586.1\|HSINTA6R Human mRNA for integrin alpha 6 | X53586 | 33943 | |
| 1736 | cr: gi\|33876397\|gb\|BC001668.2\| Homo sapiens serine protease inhibitor, Kunitz | BC001668 | 33876397 | |
| | type, 2, mRNA (cDNA clone MGC: 2021 IMAGE: 2959462), complete cds | | | |
| 1737 | Homo sapiens nucleolar protein with MIF4G domain 1, mRNA (cDNA clone | BC007902 | 33873701 | |
| | IMAGE: 4138787), partial cds | | | |
| 1738 | Homo sapiens SM-11044 binding protein (SMBP), mRNA | NM_020123 | 33859832 | SMBP |
| 1739 | cr: gi\|338270\|gb\|M36001.1\|HUMSNRNPPS Human small nuclear RNA protein | M36001 | 338270 | |
| | pseudogene, complete cds | | | |
| 1740 | Homo sapiens RAB10, member RAS oncogene family (RAB10), mRNA | NM_016131 | 33695094 | RAB10 |
| 1741 | Homo sapiens likely ortholog of mouse ubiquitin-conjugating enzyme E2-230K (E2- | NM_022066 | 33636749 | E2-230K |
| | 230K), mRNA | | | |
| 1743 | Homo sapiens tousled-like kinase 1 (TLK1), mRNA | NM_012290 | 33636697 | TLK1 |
| 1744 | Homo sapiens transmembrane channel-like 7 (TMC7), mRNA | NM_024847 | 33636690 | TMC7 |
| 1745 | Homo sapiens transformer-2 alpha (TRA2A), mRNA | NM_013293 | 33620726 | TRA2A |
| 1746 | Homo sapiens myotubularin related protein 9 (MTMR9), mRNA | NM_015458 | 33598962 | MTMR9 |
| 1747 | Homo sapiens EPH receptor B3 (EPHB3), mRNA | NM_004443 | 33598961 | EPHB3 |
| 1748 | Homo sapiens phospholipase C, gamma 1 (PLCG1), transcript variant 2, mRNA | NM_182811 | 33598945 | PLCG1 |
| 1749 | Homo sapiens bleomycin hydrolase (BLMH), mRNA | NM_000386 | 33591068 | BLMH |
| 1750 | Homo sapiens integral membrane protein 2A (ITM2A), mRNA | NM_004867 | 33589836 | ITM2A |
| 1751 | Homo sapiens eukaryotic translation initiation factor 1A, X-linked (EIF1AX), mRNA | NM_001412 | 33519477 | EIF1AX |
| 1752 | Homo sapiens Nedd4 family interacting protein 1 (NDFIP1), mRNA | NM_030571 | 33519472 | NDFIP1 |
| 1755 | Homo sapiens methionine adenosyltransferase II, beta (MAT2B), transcript variant | NM_182796 | 33519454 | MAT2B |
| | 2, mRNA | | | |
| 1756 | Homo sapiens thioredoxin reductase 1 (TXNRD1), transcript variant 5, mRNA | NM_182729 | 33519425 | TXNRD1 |
| 1757 | Homo sapiens FK506 binding protein 9, 63 kDa (FKBP9), mRNA | NM_007270 | 33469984 | FKBP9 |
| 1758 | Homo sapiens protein disulfide isomerase family A, member 4 (PDIA4), mRNA | NM_004911 | 33469983 | PDIA4 |
| 1759 | Homo sapiens RNA binding motif protein 12 (RBM12), transcript variant 2, mRNA | NM_152838 | 33469953 | RBM12 |
| 1760 | Homo sapiens MCM6 minichromosome maintenance deficient 6 (MIS5 homolog, | NM_005915 | 33469920 | MCM6 |
| | S. pombe) (S. cerevisiae) (MCM6), mRNA | | | |
| 1761 | Homo sapiens component of oligomeric golgi complex 6 (COG6), mRNA | NM_020751 | 33457343 | COG6 |
| 1762 | Homo sapiens transmembrane emp24 protein transport domain containing 4 | NM_182547 | 33457307 | TMED4 |
| | (TMED4), mRNA | | | |
| 1763 | Homo sapiens glycerophosphodiester phosphodiesterase domain containing 4 | NM_182833 | 33457300 | GDPD4 |
| | (GDPD4), mRNA | | | |
| 1764 | Homo sapiens esterase D/formylglutathione hydrolase (ESD), mRNA | NM_001984 | 33413399 | ESD |
| 1765 | Homo sapiens pinin, desmosome associated protein (PNN), mRNA | NM_002687 | 33356173 | PNN |
| 1766 | Homo sapiens myosin IXB (MYO9B), mRNA | NM_004145 | 33356169 | MYO9B |
| 1767 | Homo sapiens eukaryotic translation initiation factor 1A, Y-linked (EIF1AY), mRNA | NM_004681 | 33356162 | EIF1AY |
| 1768 | Homo sapiens KIAA1704 (KIAA1704), mRNA | NM_018559 | 33354278 | KIAA1704 |
| 1769 | Homo sapiens dynein, cytoplasmic, heavy polypeptide 1 (DNCH1), mRNA | NM_001376 | 33350931 | DNCH1 |
| 1770 | Homo sapiens FP291 mRNA, complete cds | AF370376 | 33341681 | |
| 1771 | Homo sapiens pyruvate kinase, muscle (PKM2), transcript variant 3, mRNA | NM_182471 | 33286421 | PKM2 |
| 1772 | Homo sapiens ubiquitin-conjugating enzyme E2D 2 (UBC4/5 homolog, yeast) | NM_003339 | 33188457 | UBE2D2 |
| | (UBE2D2), transcript variant 1, mRNA | | | |
| 1773 | Homo sapiens protein kinase, AMP-activated, gamma 2 non-catalytic subunit | NM_016203 | 33186924 | PRKAG2 |
| | (PRKAG2), mRNA | | | |
| 1774 | Homo sapiens ubiquitin-conjugating enzyme E2D 3 (UBC4/5 homolog, yeast) | NM_181888 | 33149313 | UBE2D3 |
| | (UBE2D3), transcript variant 4, mRNA | | | |
| 1775 | Synthetic construct human lysozyme mRNA, complete cds | AY330867 | 33088589 | |
| 1776 | Homo sapiens ES/130-related protein mRNA, partial cds | AF007575 | 3299886 | |
| 1777 | Homo sapiens ankyrin 3, node of Ranvier (ankyrin G) (ANK3), transcript variant 1, | NM_020987 | 32967600 | ANK3 |
| | mRNA | | | |
| 1778 | Homo sapiens neurofibromin 2 (bilateral acoustic neuroma) (NF2), transcript | NM_181825 | 32967253 | NF2 |
| | variant 12, mRNA | | | |
| 1779 | Homo sapiens collagen, type VIII, alpha 1 (COL8A1), transcript variant 2, mRNA | NM_020351 | 32895367 | COL8A1 |
| 1780 | Homo sapiens hypothetical protein FLJ34969 (FLJ34969), mRNA | NM_152678 | 32698776 | FLJ34969 |
| 1781 | Homo sapiens arrestin domain containing 3 (ARRDC3), mRNA | NM_020801 | 32698735 | ARRDC3 |
| 1782 | Homo sapiens SR protein (RNPS1) mRNA, complete cds | AF015608 | 3253164 | RNPS1 |
| 1783 | Homo sapiens GATA binding protein 1 (globin transcription factor 1) (GATA1), | NM_002049 | 32483408 | GATA1 |
| | mRNA | | | |
| 1784 | Homo sapiens CD19 antigen (CD19), mRNA | NM_001770 | 32481214 | CD19 |
| 1785 | Homo sapiens peroxiredoxin 1 (PRDX1), transcript variant 3, mRNA | NM_181697 | 32455265 | PRDX1 |
| 1787 | Homo sapiens serpin peptidase inhibitor, clade H (heat shock protein 47), member | NM_001235 | 32454740 | SERPINH1 |
| | 1, (collagen binding protein 1) (SERPINH1), mRNA | | | |
| 1788 | Homo sapiens solute carrier family 36 (proton/amino acid symporter), member 2 | NM_181776 | 32401452 | SLC36A2 |
| | (SLC36A2), mRNA | | | |
| 1789 | Homo sapiens sprouty-related, EVH1 domain containing 2 (SPRED2), mRNA | NM_181784 | 32401444 | SPRED2 |
| 1790 | Homo sapiens MARCKS-like 1 (MARCKSL1), mRNA | NM_023009 | 32401423 | MARCKSL1 |
| 1791 | Homo sapiens synaptoporin (SYNPR), mRNA | NM_144642 | 32401419 | SYNPR |
| 1792 | Homo sapiens hypothetical protein MGC24039 (MGC24039), mRNA | NM_144973 | 32307180 | MGC24039 |
| 1793 | Homo sapiens cullin 1 (CUL1), mRNA | NM_003592 | 32307160 | CUL1 |
| 1794 | Homo sapiens centaurin, gamma 3 (CENTG3), mRNA | NM_031946 | 32307157 | CENTG3 |
| 1795 | Homo sapiens procollagen-lysine 1,2-oxoglutarate 5-dioxygenase 1 (PLOD1), | NM_000302 | 32307143 | PLOD1 |
| | mRNA | | | |
| 1796 | Homo sapiens protein phosphatase 2 (formerly 2A), regulatory subunit B (PR 52), | NM_004576 | 32307122 | PPP2R2B |
| | beta isoform (PPP2R2B), transcript variant 1, mRNA | | | |
| 1797 | Homo sapiens protein phosphatase 2 (formerly 2A), regulatory subunit B (PR 52), | NM_002717 | 32307111 | PPP2R2A |
| | alpha isoform (PPP2R2A), mRNA | | | |
| 1798 | Homo sapiens unc-5 homolog B (C. elegans) (UNC5B), mRNA | NM_170744 | 32261317 | UNC5B |
| 1799 | Homo sapiens maternal G10 transcript (G10), mRNA | NM_003910 | 32171174 | G10 |
| 1800 | Homo sapiens adenylosuccinate lyase (ADSL) mRNA, alternatively spliced, | AF067854 | 3211983 | ADSL |
| | complete cds | | | |
| 1801 | Homo sapiens jagunal homolog 1 (Drosophila) (JAGN1), mRNA | NM_032492 | 31982910 | JAGN1 |
| 1802 | Homo sapiens small acidic protein (SMAP), mRNA | NM_014267 | 31982890 | SMAP |
| 1803 | Homo sapiens GATA binding protein 2 (GATA2), mRNA | NM_032638 | 31982886 | GATA2 |
| 1804 | Homo sapiens PIN2-interscting protein 1 (PINX1), mRNA | NM_017884 | 31982866 | PINX1 |
| 1805 | Homo sapiens serine palmitoyltransferase, long chain base subunit 2 (SPTLC2), | NM_004863 | 31881646 | SPTLC2 |
| | mRNA | | | |
| 1806 | Homo sapiens mRNA; cDNA DKFZp779B0244 (from clone DKFZp779B0244) | BX538099 | 31874241 | |
| 1807 | Homo sapiens keratin associated protein 6-1 (KRTAP6-1), mRNA | NM_181602 | 31791037 | KRTAP6-1 |
| 1808 | Human mRNA for G(i) protein alpha-subunit (adenylate cyclase inhibiting GTP- | X04828 | 31743 | I |
| | binding protein) | | | |
| 1809 | Homo sapiens leucine-rich PPR-motif containing (LRPPRC), mRNA | NM_133259 | 31621304 | LRPPRC |
| 1810 | Homo sapiens myosin, light polypeptide 9, regulatory (MYL9), transcript variant 1, | NM_006097 | 31563522 | MYL9 |
| | mRNA | | | |
| 1811 | Homo sapiens integrin beta 4 binding protein (ITGB4BP), transcript variant 2, | NM_181468 | 31563377 | ITGB4BP |
| | mRNA | | | |
| 1812 | Homo sapiens A kinase (PRKA) anchor protein 13 (AKAP13), transcript variant 2, | NM_007200 | 31563331 | PRKA |
| | mRNA | | | |
| 1813 | Homo sapiens TSC22 domain family, member 1 (TSC22D1), transcript variant 2, | NM_006022 | 31543826 | TSC22D1 |
| | mRNA | | | |
| 1814 | Homo sapiens stomatin (EPB72)-like 1 (STOML1), mRNA | NM_004809 | 31543664 | EPB72 |
| 1815 | Homo sapiens signal recognition particle 14 kDa (homologous Alu RNA binding | NM_003134 | 31543652 | SRP14 |
| | protein) (SRP14), mRNA | | | |
| 1816 | Homo sapiens solute carrier family 20 (phosphate transporter), member 1 | NM_005415 | 31543829 | SLC20A1 |
| | (SLC20A1), mRNA | | | |
| 1817 | Homo sapiens px19-like protein (PX19), mRNA | NM_013237 | 31543450 | PX19 |
| 1818 | Homo sapiens pim-1 oncogene (PIM1), mRNA | NM_002648 | 31543400 | PIM1 |
| 1819 | Homo sapiens phosphoglycerate mutase 1 (brain) (PGAM1), mRNA | NM_002629 | 31543395 | PGAM1 |
| 1820 | Homo sapiens pyruvate dehydrogenase kinase, isoenzyme 2 (PDK2), mRNA | NM_002611 | 31543388 | PDK2 |
| 1821 | Homo sapiens ninjurin 1 (NINJ1), mRNA | NM_004148 | 31543289 | NINJ1 |
| 1822 | Homo sapiens metallothionein 2A (MT2A), mRNA | NM_005953 | 31543214 | MT2A |
| 1823 | Homo sapiens chromosome 7 open reading frame 21 (C7orf21), mRNA | NM_031434 | 31543199 | C7orf21 |
| 1824 | Homo sapiens serine/threonine kinase 40 (STK40), mRNA | NM_032017 | 31543197 | STK40 |
| 1825 | Homo sapiens ring finger protein 149 (RNF149), mRNA | NM_173647 | 31543079 | RNF149 |
| 1826 | Homo sapiens guanosine monophosphate reductase (GMPR), mRNA | NM_006877 | 31542848 | GMPR |
| 1827 | Homo sapiens chromosome 2 open reading frame 18 (C2orf18), mRNA | NM_017877 | 31542710 | C2orf18 |
| 1828 | Homo sapiens NECAP endocytosis associated 1 (NECAP1), mRNA | NM_015509 | 31542527 | NECAP1 |
| 1829 | Homo sapiens chromosome 14 open reading frame 10 (C14orf10), mRNA | NM_017917 | 31542241 | C14orf10 |
| 1830 | Homo sapiens alpha-1-microglobulin/bikunin precursor (AMBP), mRNA | NM_001633 | 31541901 | AMBP |
| 1831 | H. sapiens mRNA for Drosophila female sterile homeotic (FSH) homologue | X62083 | 31471 | FSH |
| 1832 | Homo sapiens mRNA for putative NFkB activating protein, complete cds, clone: | AB097007 | 31455468 | |
| | 120 | | | |
| 1833 | Homo sapiens exosome component 8 (EXOSC8), mRNA | NM_181503 | 31415881 | EXOSC8 |
| 1834 | Homo sapiens calmodulin 1 (phosphorylase kinase, delta) (CALM1), mRNA | NM_006888 | 31377794 | CALM1 |
| 1835 | Homo sapiens phosphatidylinositol transfer protein, alpha (PITPNA), mRNA | NM_006224 | 31377785 | PITPNA |
| 1836 | Homo sapiens glycosyltransferase 25 domain containing 1 (GLT25D1), mRNA | NM_024656 | 31377696 | GLT25D1 |
| 1837 | Homo sapiens splicing factor, arginine/serine-rich 3 (SFRS3), mRNA | NM_003017 | 31377552 | SFRS3 |
| 1838 | Homo sapiens prenylcysteine carboxyl methyltransferase (PCCMT) mRNA, | AF064084 | 3135668 | PCCMT |
| | complete cds | | | |
| 1839 | Homo sapiens smooth muscle myosin heavy chain 11 isoform SM1-like | NM_152994 | 31343495 | LOC129285 |
| | (LOC129285), mRNA | | | |
| 1840 | Homo sapiens NS5ATP13TP2 protein (NS5ATP13TP2), mRNA | NM_178507 | 31341888 | NS5ATP13TP2 |
| 1841 | Homo sapiens hypothetical protein MGC33407 (MGC33407), mRNA | NM_178525 | 31341825 | MGC33407 |
| 1842 | Homo sapiens hypothetical protein MGC50721 (MGC50721), mRNA | NM_173806 | 31341127 | MGC50721 |
| 1843 | Homo sapiens inhibitor of DNA binding 1, dominant negative helix-loop-helix | NM_002165 | 31317298 | ID1 |
| | protein (ID1), transcript variant 1, mRNA | | | |
| 1844 | Homo sapiens inhibitor of DNA binding 1, dominant negative helix-loop-helix | NM_181353 | 31317296 | ID1 |
| | protein (ID1), transcript variant 2, mRNA | | | |
| 1845 | Homo sapiens tafazzin (cardiomyopathy, dilated 3A (X-linked); endocardial | NM_181311 | 31317258 | TAZ |
| | fibroelastosis 2; Barth syndrome) (TAZ), transcript variant 2, mRNA | | | |
| 1846 | Homo sapiens early growth response 1 (EGR1), mRNA | NM_001964 | 31317226 | EGR1 |
| 1847 | Homo sapiens ephrin-B1 (EFNB1), mRNA | NM_004429 | 31317225 | EFNB1 |
| 1848 | Human ERK1 mRNA for protein serine/threonine kinase | X60188 | 31220 | ERK1 |
| 1849 | Homo sapiens protein phosphatase 2, regulatory subunit B (B56), delta isoform | NM_006245 | 31083266 | PPP2R5D |
| | (PPP2R5D), transcript variant 1, mRNA | | | |
| 1850 | Homo sapiens CCR4 carbon catabolite repression 4-like (S. cerevisiae) | NM_012118 | 31083027 | CCRN4L |
| | (CCRN4L), mRNA | | | |
| 1851 | Homo sapiens brain abundant, membrane attached signal protein 1 (BASP1), | NM_006317 | 30795230 | BASP1 |
| | mRNA | | | |
| 1852 | cr: gi\|30583494\|gb\|BT007328.1\| Homo sapiens LIM protein (similar to rat protein | BT007328 | 30583494 | |
| | kinase C-binding enigma) mRNA, complete cds | | | |
| 1853 | Homo sapiens replication factor C (activator 1) 2, 40 kDa mRNA, complete cds | BT007058 | 30582954 | |
| 1854 | Homo sapiens proteasome (prosome, macropain) activator subunit 1 (PA28 alpha) | NM_006263 | 30581139 | PSME1 |
| | (PSME1), transcript variant 1, mRNA | | | |
| 1855 | Homo sapiens SRY (sex determining region Y)-box 11 (SOX11), mRNA | NM_003108 | 30581115 | SOX11 |
| 1856 | Homo sapiens source of immunodominant MHC-associated peptides (SIMP), | NM_178862 | 30578409 | SIMP |
| | mRNA | | | |
| 1857 | Homo sapiens TBC1 domain family, member 8 (with GRAM domain) (TBC1D8), | NM_007063 | 30410773 | TBC1D8 |
| | mRNA | | | |
| 1858 | Homo sapiens mRNA for phospholipase C-alpha, complete cds | D16234 | 303617 | |
| 1859 | Homo sapiens spectrin, beta, non-erythrocytic 1 (SPTBN1), transcript variant 2, | NM_178313 | 30315657 | SPTBN1 |
| | mRNA | | | |
| 1860 | Homo sapiens mRNA; cDNA DKFZp451F1115 (from clone DKFZp451F1115) | AL833062 | 30268348 | |
| 1861 | Homo sapiens SRY (sex determining region Y)-box 4 (SOX4), mRNA | NM_003107 | 30179901 | SOX4 |
| 1862 | Homo sapiens troponin I, cardiac (TNNI3), mRNA | NM_000363 | 30141909 | TNNI3 |
| 1863 | Homo sapiens acid phosphatase 1, soluble (ACP1), transcript variant 2, mRNA | NM_007099 | 30090001 | ACP1 |
| 1864 | Homo sapiens actin-like 6A (ACTL6A), transcript variant 1, mRNA | NM_004301 | 30089995 | ACTL6A |
| 1865 | Homo sapiens CDC42 effector protein (Rho GTPase binding) 3 (CDC42EP3), | NM_006449 | 30089964 | CDC42EP3 |
| | mRNA | | | |
| 1866 | Homo sapiens N-acylsphingosine amidohydrolase (acid ceramidase) 1 (ASAH1), | NM_004315 | 30089929 | ASAH1 |
| | transcript variant 2, mRNA | | | |
| 1867 | Homo sapiens F-box protein 16 (FBXO16), mRNA | NM_172366 | 30089920 | FBXO16 |
| 1868 | Homo sapiens phosphofurin acidic cluster sorting protein 1 (PACS1), mRNA | NM_018026 | 30089915 | PACS1 |
| 1869 | Homo sapiens meteorin, glial cell differentiation regulator-like, mRNA (cDNA clone | BC050568 | 30047762 | |
| | IMAGE: 5213890), partial cds | | | |
| 1870 | Homo sapiens stannin (SNN), mRNA | NM_003498 | 29893560 | SNN |
| 1871 | Homo sapiens chromosome 1 open reading frame 9 (C1orf9), transcript variant 1, | NM_014283 | 29837653 | C1orf9 |
| | mRNA | | | |
| 1872 | Homo sapiens regulator of chromosome condensation 2 (RCC2), mRNA | NM_018715 | 29789089 | RCC2 |
| 1873 | Homo sapiens pleckstrin homology domain containing, family C (with FERM | NM_006832 | 29789005 | PLEKHC1 |
| | domain) member 1 (PLEKHC1), mRNA | | | |
| 1874 | Homo sapiens phosphoribosyl pyrophosphate amidotransferase (PPAT), mRNA | NM_002703 | 29570797 | PPAT |
| 1875 | Homo sapiens catenin, beta like 1 (CTNNBL1), mRNA | NM_030877 | 29570786 | CTNNBL1 |
| 1876 | Homo sapiens dynein, cytoplasmic, light polypeptide 2A (DNCL2A), transcript | NM_014183 | 29570778 | DNCL2A |
| | variant 1, mRNA | | | |
| 1877 | Homo sapiens Rho GTPase-activating protein (RICS), mRNA | NM_014715 | 29469070 | RICS |
| 1878 | Homo sapiens mesoderm specific transcript homolog (mouse) (MEST), transcript | NM_002402 | 29294638 | MEST |
| | variant 1, mRNA | | | |
| 1879 | Homo sapiens small nuclear ribonucleoprotein D2 polypeptide 16.5 kDa (SNRPD2), | NM_004597 | 29294622 | SNRPD2 |
| | transcript variant 1, mRNA | | | |
| 1880 | Homo sapiens phosphatidic acid phosphatase type 2B (PPAP2B), transcript | NM_003713 | 29171739 | PPAP2B |
| | variant 1, mRNA | | | |
| 1881 | Homo sapiens cytochrome P450, family 20, subfamily A, polypeptide 1 | NM_177538 | 29171729 | CYP20A1 |
| | (CYP20A1), transcript variant 1, mRNA | | | |
| 1882 | Homo sapiens melanoma antigen family D, 2 (MAGED2), transcript variant 2, | NM_177433 | 29171704 | MAGED2 |
| | mRNA | | | |
| 1883 | Homo sapiens ras homolog gene family, member J (RHOJ), mRNA | NM_020663 | 29171294 | RHOJ |
| 1884 | Homo sapiens hypothetical protein FLJ10726, mRNA (cDNA clone MGC: 57486 | BC048133 | 29126862 | |
| | IMAGE: 5295844), complete cds | | | |
| 1885 | Homo sapiens arsenite translocating ATPase (ASNA1) mRNA, complete cds | AF047469 | 2905656 | ASNA1 |
| 1886 | H. sapiens mRNA for autoantigen NOR-90 | X56687 | 28970 | |
| 1887 | Homo sapiens GC-rich promoter binding protein 1 (GPBP1), mRNA | NM_022913 | 28875781 | GPBP1 |
| 1889 | Homo sapiens CDK5 regulatory subunit associated protein 3 (CDK5RAP3), | NM_176096 | 28872791 | CDK5RAP3 |
| | transcript variant 3, mRNA | | | |
| 1890 | Homo sapiens CDK5 regulatory subunit associated protein 1 (CDK5RAP1), | NM_016408 | 28872781 | CDK5RAP1 |
| | transcript variant 1, mRNA | | | |
| 1891 | Homo sapiens BTG family, member 2 (BTG2), mRNA | NM_006763 | 28872718 | BTG2 |
| 1892 | Homo sapiens recombination activating gene 2 (RAG2), mRNA | NM_000536 | 28629867 | RAG2 |
| 1893 | Homo sapiens reticulocalbin 3, EF-hand calcium binding domain (RCN3), mRNA | NM_020650 | 28626509 | RCN3 |
| 1894 | Homo sapiens interleukin 7 receptor (IL7R), mRNA | NM_002185 | 28610150 | IL7R |
| 1895 | Homo sapiens interleukin 6 signal transducer (gp130, oncostatin M receptor) | NM_002184 | 28610146 | IL6ST |
| | (IL6ST), transcript variant 1, mRNA | | | |
| 1896 | Homo sapiens peroxiredoxin 6 (PRDX6), mRNA | NM_004905 | 28559000 | PRDX6 |
| 1897 | Homo sapiens polypyrimidine tract binding protein 1 (PTBP1), transcript variant 2, | NM_031990 | 28558995 | PTBP1 |
| | mRNA | | | |
| 1898 | Homo sapiens polypyrimidine tract binding protein 1 (PTBP1), transcript variant 1, | NM_002819 | 28558994 | PTBP1 |
| | mRNA | | | |
| 1899 | Homo sapiens structure specific recognition protein 1 (SSRP1), mRNA | NM_003146 | 28416943 | SSRP1 |
| 1900 | Homo sapiens signal sequence receptor, gamma (translocon-associated protein | NM_007107 | 28416942 | SSR3 |
| | gamma) (SSR3), mRNA | | | |
| 1901 | human full-length cDNA 5-PRIME end of clone CS0DF007YN24 of Fetal brain of | BX248753 | 28375482 | |
| | Homo sapiens (human) | | | |
| 1902 | Homo sapiens nidogen 1, mRNA (cDNA clone MGC: 33141 IMAGE: 5271590), | BC045606 | 28374138 | |
| | complete cds | | | |
| 1903 | Homo sapiens ST3 beta-galactoside alpha-2,3-sialyltransferase 5 (ST3GAL5), | NM_003896 | 28373079 | ST3GAL5 |
| | mRNA | | | |
| 1904 | Homo sapiens hypothetical protein LOC201895 (LOC201895), mRNA | NM_174921 | 28372538 | LOC201895 |
| 1905 | Homo sapiens protein phosphatase 4, regulatory subunit 2 (PPP4R2), mRNA | NM_174907 | 28372530 | PPP4R2 |
| 1906 | Homo sapiens TGFB-induced factor (TALE family homeobox) (TGIF), transcript | NM_003244 | 28178841 | TGIF |
| | variant 4, mRNA | | | |
| 1908 | Homo sapiens isocitrate dehydrogenase 1 (NADP+), soluble (IDH1), mRNA | NM_005896 | 28178824 | IDH1 |
| 1910 | Homo sapiens calsenilin, presenilin binding protein, EF-hand transcription factor | NM_013434 | 27894379 | CSEN |
| | (CSEN), mRNA | | | |
| 1911 | Homo sapiens msh homeo box homolog 2 (Drosophila) (MSX2), mRNA | NM_002449 | 27886556 | MSX2 |
| 1912 | Homo sapiens ATPase, Ca++ transporting, cardiac muscle, slow twitch 2 | NM_001681 | 27886536 | ATP2A2 |
| | (ATP2A2), transcript variant 2, mRNA | | | |
| 1913 | Homo sapiens nuclear factor of activated T-cells 5, tonicity-responsive (NFAT5), | NM_138714 | 27886519 | NFAT5 |
| | transcript variant 1, mRNA | | | |
| 1914 | Homo sapiens ras-related C3 botulinum toxin substrate 2 (rho family, small GTP | NM_002872 | 27881480 | RAC2 |
| | binding protein Rac2) (RAC2), mRNA | | | |
| 1915 | Homo sapiens solute carrier family 9 (sodium/hydrogen exchanger), isoform 1 | NM_003047 | 27777631 | SLC9A1 |
| | (antiporter, Na+/H+, amiloride sensitive) (SLC9A1), mRNA | | | |
| 1916 | Homo sapiens cDNA clone IMAGE: 4820027, containing frame-shift errors | BC042460 | 27769042 | |
| 1917 | Homo sapiens myosin, heavy polypeptide 6, cardiac muscle, alpha | NM_002471 | 27764860 | MYH6 |
| | cardiomyopathy, hypertrophic 1) (MYH6), mRNA | | | |
| 1918 | Homo sapiens afamin (AFM), mRNA | NM_001133 | 27754774 | AFM |
| 1919 | Homo sapiens DTW domain containing 2 (DTWD2), mRNA | NM_173666 | 27734778 | DTWD2 |
| 1920 | Homo sapiens hypothetical protein FLJ90652 (FLJ90652), mRNA | NM_173618 | 27734726 | FLJ90652 |
| 1921 | Homo sapiens leucine zipper protein 5, mRNA (cDNA clone MGC: 50909 | BC043404 | 27695960 | |
| | IMAGE: 6045248), complete cds | | | |
| 1922 | Homo sapiens methionyl aminopeptidase 2 (METAP2), mRNA | NM_006838 | 27597083 | METAP2 |
| 1923 | Homo sapiens ubiquitin protein ligase E3 component n-recognin 2 (UBR2), mRNA | NM_015255 | 27597060 | UBR2 |
| 1924 | cand_Human_STR_34C6_26Jun2003.fa BaseSame: gi\|27503691\|gb\|BC042478.1\| | BC042478 | 27503691 | |
| | Homo sapiens hypothetical protein DKFZp434F0318, clone IMAGE: 3874052, | | | |
| | mRNA, partial cds | | | |
| 1926 | Homo sapiens cervical cancer oncogene 9 mRNA, complete cds | AY071927 | 27461089 | |
| 1929 | Homo sapiens extracellular sulfatase SULF-2 mRNA, complete cds | AY101176 | 27356933 | |
| 1930 | Homo sapiens Tle-2 mRNA for receptor protein tyrosine kinase, partial cds | AB086825 | 27348225 | |
| 1932 | Homo sapiens adenylate kinase 2 (AK2), transcript variant AK2B, mRNA | NM_013411 | 26665888 | AK2B |
| 1933 | Homo sapiens cytochrome oxidase subunit I (COI) and subunit II (COII) | AF035429 | 2665724 | COI |
| | pseudogenes, complete sequence | | | |
| 1934 | Homo sapiens potassium voltage-gated channel, subfamily H (eag-related), | NM_000238 | 26051269 | KCNH2 |
| | member 2 (KCNH2), transcript variant 1, mRNA | | | |
| 1935 | Homo sapiens tumor necrosis factor, alpha-induced protein 6 (TNFAIP6), mRNA | NM_007115 | 26051242 | TNFAIP6 |
| 1936 | Homo sapiens tumor necrosis factor, alpha-induced protein 1 (endothelial) | NM_021137 | 26051238 | TNFAIP1 |
| | (TNFAIP1), mRNA | | | |
| 1937 | Homo sapiens nucleoporin 54 kDa (NUP54), mRNA | NM_017426 | 26051236 | NUP54 |
| 1938 | Homo sapiens transportin2 mRNA, complete cds | AF019039 | 2589203 | |
| 1939 | cr: gi\|2586349\|gb\|AF022813.1\|AF022813 Homo sapiens tetraspan (NAG-2) | AF022813 | 2586349 | NAG-2 |
| | mRNA, complete cds | | | |
| 1942 | Homo sapiens aldehyde dehydrogenase 1 family, member A1 (ALDH1A1), mRNA | NM_000689 | 25777722 | ALDH1A1 |
| 1943 | Homo sapiens zinc finger protein 91 homolog (mouse) (ZFP91), transcript variant | NM_053023 | 25777699 | ZFP91 |
| | 1, mRNA | | | |
| 1944 | Homo sapiens Ras association (RalGDS/AF-6) domain family 1 (RASSF1), | NM_170713 | 25777681 | RASSF1 |
| | transcript variant C, mRNA | | | |
| 1945 | Homo sapiens Ras association (RalGDS/AF-6) domain family 1 (RASSF1), | NM_170712 | 25777679 | RASSF1 |
| | transcript variant B, mRNA | | | |
| 1946 | Homo sapiens proteasame (prosome, macropain) 26S subunit, non-ATPase, 3 | NM_002809 | 25777611 | PSMD3 |
| | (PSMD3), mRNA | | | |
| 1947 | Homo sapiens proteasome (prosome, macropain) 26S subunit, non-ATPase, 2 | NM_002808 | 25777601 | PSMD2 |
| | (PSMD2), mRNA | | | |
| 1948 | Homo sapiens proteasome (prosome, macropain) 26S subunit, non-ATPase, 1 | NM_002807 | 25777599 | PSMD1 |
| | (PSMD1), mRNA | | | |
| 1949 | Homo sapiens eukaryotic translation initiation factor (eIF3) mRNA, complete cds | U78525 | 2558667 | eIF3 |
| 1950 | Homo sapiens DAZ associated protein 1 (DAZAP1), transcript variant 1, mRNA | NM_170711 | 25470889 | DAZAP1 |
| 1951 | Homo sapiens, Similar to selectin, endothelial cell, ligand, clone IMAGE: 4400215, | BC040133 | 25455703 | |
| | mRNA | | | |
| 1952 | Homo sapiens coenzyme Q7 homolog, ubiquinone (yeast) (COQ7), mRNA | NM_016138 | 25453483 | COQ7 |
| 1953 | Homo sapiens eukaryotic translation elongation factor 1 gamma (EEF1G), mRNA | NM_001404 | 25453475 | EEF1G |
| 1954 | Homo sapiens eukaryotic translation elongation factor 1 alpha 1 (EEF1A1), mRNA | NM_001402 | 25453469 | EEF1A1 |
| 1956 | Homo sapiens interferon-stimulated transcription factor 3, gamma 48 kDa | NM_006084 | 25282406 | ISGF3G |
| | (ISGF3G), mRNA | | | |
| 1957 | Homo sapiens voltage-dependent anion channel 3 (VDAC3), mRNA | N_005662 | 25188178 | VDAC3 |
| 1958 | Homo sapiens serum/glucocorticoid regulated kinase (SGK), mRNA | NM_005627 | 25168262 | SGK |
| 1959 | Homo sapiens barren homolog (Drosophila) (BRRN1), mRNA | NM_015341 | 25121986 | BRRN1 |
| 1960 | Homo sapiens selenophosphate synthetase 2 (SEPHS2), mRNA | NM_012248 | 24797146 | SEPHS2 |
| 1961 | Homo sapiens homeo box B9 (HOXB9), mRNA | NM_024017 | 24797138 | HOXB9 |
| 1962 | Homo sapiens karyopherin (importin) beta 1 (KPNB1), mRNA | NM_002265 | 24797084 | KPNB1 |
| 1963 | Homo sapiens integrin beta 1 binding protein 3 (ITGB1BP3), transcript variant 2, | NM_170678 | 24762247 | ITGB1BP3 |
| | mRNA | | | |
| 1964 | Homo sapiens, Similar to bromodomain containing 4, clone IMAGE: 5787152, | BC038988 | 24658603 | |
| | mRNA | | | |
| 1965 | Homo sapiens Down syndrome critical region gene 5 (DSCR5), transcript variant | NM_153682 | 24497598 | DSCR5 |
| | 2, mRNA | | | |
| 1966 | Homo sapiens Down syndrome critical region gene 5 (DSCR5), transcript variant | NM_153681 | 24497596 | DSCR5 |
| | 1, mRNA | | | |
| 1967 | Homo sapiens endothelial differentiation-related factor 1 (EDF1), transcript variant | NM_003792 | 24497592 | EDF1 |
| | alpha, mRNA | | | |
| 1968 | Homo sapiens adenylate cyclase 4 (ADCY4), mRNA | NM_139247 | 24497586 | ADCY4 |
| 1969 | Homo sapiens aldo-keto reductase family 1, member C4 (chlordecone reductase; | NM_001818 | 24497584 | AKR1C4 |
| | 3-alpha hydroxysteroid dehydrogenase, type I; dihydrodiol dehydrogenase 4) | | | |
| | (AKR1C4), mRNA | | | |
| 1970 | Homo sapiens solute carrier family 22 (extraneuronal monoamine transporter), | NM_021977 | 24497488 | SLC22A3 |
| | member 3 (SLC22A3), mRNA | | | |
| 1971 | Homo sapiens fucosidase, alpha-L-1, tissue (FUCA1), mRNA | NM_000147 | 24475878 | FUCA1 |
| 1972 | Homo sapiens RING1 and YY1 binding protein (RYBP), mRNA | NM_012234 | 24432049 | RYBP |
| 1973 | Homo sapiens chromatin accessibility complex 1 (CHRAC1), mRNA | NM_017444 | 24432041 | CHRAC1 |
| 1974 | Homo sapiens outer dense fiber of sperm tails 1 (ODF1), mRNA | NM_024410 | 24430179 | ODF1 |
| 1975 | Homo sapiens peripheral myelin protein 22 (PMP22), transcript variant 1, mRNA | NM_000304 | 24430161 | PMP22 |
| 1976 | Homo sapiens proteasome (prosome, macropain) 26S subunit, ATPase, 6 | NM_002806 | 24430159 | PSMC6 |
| | (PSMC6), mRNA | | | |
| 1977 | Homo sapiens fibrillin 1 (Marfan syndrome) (FBN1), mRNA | NM_000138 | 24430140 | FBN1 |
| 1978 | Homo sapiens proline-, glutamic acid-, leucine-rich protein 1 (PELP1), mRNA | NM_014389 | 24415382 | PELP1 |
| 1979 | Homo sapiens ADP-ribosylation factor-like 6 interacting protein (ARL6IP), mRNA | NM_015161 | 24308006 | ARL6IP |
| 1980 | Homo sapiens putative nucleic acid binding protein RY-1 (RY1), mRNA | NM_006857 | 24307918 | RY1 |
| 1981 | Homo sapiens dynein, cytoplasmic, intermediate polypeptide 2 (DNCI2), mRNA | NM_001378 | 24307878 | DNCI2 |
| 1982 | Homo sapiens zinc finger protein 11B (ZNF11B), mRNA | NM_006955 | 24307874 | ZNF11B |
| 1983 | Homo sapiens sorting nexin 8 (SNX8), mRNA | NM_013321 | 23943857 | SNX8 |
| 1984 | Homo sapiens sin3-associated polypeptide, 18 kDa (SAP18), mRNA | NM_005870 | 23510407 | SAP18 |
| 1985 | Homo sapiens ubiquitin-activating enzyme E1 (A1S9T and BN75 temperature | NM_003334 | 23510337 | UBE1 |
| | sensitivity complementing) (UBE1), transcript variant 1, mRNA | | | |
| 1986 | Homo sapiens chromosome 10 open reading frame 56 (C10orf56), mRNA | NM_153367 | 23503314 | C10orf56 |
| 1987 | Homo sapiens acidic (leucine-rich) nuclear phosphoprotein 32 family, member E | NM_030920 | 23463320 | ANP32E |
| | (ANP32E), mRNA | | | |
| 1988 | Homo sapiens copine I (CPNE1), transcript variant 8, mRNA | NM_152931 | 23397707 | CPNE1 |
| 1989 | Homo sapiens actin related protein 2/3 complex, subunit 3, 21 kDa (ARPC3), | NM_005719 | 23397667 | ARPC3 |
| | mRNA | | | |
| 1990 | Homo sapiens phosphatidylinositol glycan, class T (PIGT), mRNA | NM_015937 | 23397652 | PIGT |
| 1991 | Homo sapiens synaptotagmin binding, cytoplasmic RNA interacting protein | NM_006372 | 23397426 | SYNCRIP |
| | (SYNCRIP), mRNA | | | |
| 1992 | Homo sapiens TGF-beta induced apotosis protein 2 (TAIP-2), mRNA | NM_024969 | 23346411 | TAIP-2 |
| 1993 | Homo sapiens chromosome 20 open reading frame 111 (C20orf111), mRNA | NM_016470 | 23346408 | C20orf111 |
| 1994 | Homo sapiens prostaglandin E synthase 3 (cytosolic) (PTGES3), mRNA | NM_006601 | 23308578 | PTGES3 |
| 1995 | Homo sapiens cDNA clone MGC: 33718 IMAGE: 5288990, complete cds | BC036074 | 23243316 | |
| 1996 | Homo sapiens, clone IMAGE: 5241630, mRNA | BC035570 | 23242693 | |
| 1997 | Homo sapiens eukaryotic translation initiation factor 3, subunit 7 zeta, 66/67 kDa | NM_003753 | 23238220 | EIF3S7 |
| | (EIF3S7), mRNA | | | |
| 1998 | Homo sapiens actin related protein 2/3 complex, subunit 2, 34 kDa (ARPC2), | NM_152862 | 23238210 | ARPC2 |
| | transcript variant 1, mRNA | | | |
| 1999 | Homo sapiens actin related protein 2/3 complex, subunit 2, 34 kDa (ARPC2), | NM_005731 | 23238209 | ARPC2 |
| | transcript variant 2, mRNA | | | |
| 2000 | Homo sapiens unc-51-like kinase 2 (C. elegans) (ULK2), mRNA | NM_014683 | 23199984 | ULK2 |
| 2001 | Homo sapiens myogenic factor 3 (MYOD1), mRNA | NM_002478 | 23111008 | MYOD1 |
| 2002 | Homo sapiens cathepsin S (CTSS), mRNA | NM_004079 | 23110961 | CTSS |
| 2003 | Homo sapiens cathepsin H (CTSH), transcript variant 1, mRNA | NM_004390 | 23110954 | CTSH |
| 2004 | Homo sapiens cathepsin D (lysosomal aspartyl peptidase) (CTSD), mRNA | NM_001909 | 23110949 | CTSD |
| 2005 | Homo sapiens proteasome (prosome, macropain) subunit, alpha type, 7 (PSMA7), | NM_002792 | 23110945 | PSMA7 |
| | transcript variant 1, mRNA | | | |
| 2006 | Homo sapiens proteasome (prosome, macropain) subunit, alpha type, 3 (PSMA3), | NM_002788 | 23110937 | PSMA3 |
| | transcript variant 1, mRNA | | | |
| 2007 | Homo sapiens proteasome (prosome, macropain) subunit, beta type, 6 (PSMB6), | NM_002798 | 23110924 | PSMB6 |
| | mRNA | | | |
| 2008 | Homo sapiens proteasome (prosome, macropain) subunit, beta type, 10 | NM_002801 | 23110923 | PSMB10 |
| | (PSMB10), mRNA | | | |
| 2009 | Homo sapiens cDNA FLJ90810 fis, clone Y79AA1000876, weakly similar to | AK075291 | 22761284 | |
| | PROTEIN DISULFIDE ISOMERASE-RELATED PROTEIN PRECURSOR | | | |
| 2010 | Homo sapiens cDNA FLJ90670 fis, clone PLACE1005539, similar to ACTIN | AK075151 | 22761054 | |
| | POLYMERIZATION INHIBITOR | | | |
| 2011 | Homo sapiens RasGEF domain family, member 1B (RASGEF1B), mRNA | NM_152545 | 22749128 | RASGEF1B |
| 2012 | Homo sapiens essential meiotic endonuclease 1 homolog 1 (S. pombe) (EME1), | NM_152463 | 22748968 | EME1 |
| | mRNA | | | |
| 2013 | Homo sapiens lactate dehydrogenase B (LDHB), mRNA | NM_002300 | 22726178 | LDHB |
| 2014 | Homo sapiens peptidylprolyl isomerase (cyclophilin)-like 2 (PPIL2), transcript | NM_148176 | 22547214 | PPIL2 |
| | variant 3, mRNA | | | |
| 2015 | Homo sapiens Zic family member 2 (odd-paired homolog, Drosophila) (ZIC2), | NM_007129 | 22547196 | ZIC2 |
| | mRNA | | | |
| 2016 | Homo sapiens frizzled homolog 4 (Drosophila) (FZD4), mRNA | NM_012193 | 22547160 | FZD4 |
| 2017 | Homo sapiens flightless I homolog (Drosophila) (FLII), mRNA | NM_002018 | 22547155 | FLII |
| 2019 | Homo sapiens proteasome (prosome, macropain) subunit, beta type, 2 (PSMB2), | NM_002794 | 22538463 | PSMB2 |
| | mRNA | | | |
| 2020 | Homo sapiens cathepsin C (CTSC), transcript variant 1, mRNA | NM_001814 | 22538438 | CTSC |
| 2021 | Homo sapiens AT rich interactive domain 5B (MRF1-like), mRNA (cDNA clone | BC036831 | 22477878 | MRF1-like |
| | IMAGE: 5529276), partial cds | | | |
| 2022 | Homo sapiens chromosome 9 open reading frame 24 (C9orf24), transcript variant | NM_032596 | 22219472 | C9orf24 |
| | 1, mRNA | | | |
| 2023 | Homo sapiens high mobility group AT-hook 1 (HMGA1), transcript variant 1, mRNA | NM_145899 | 22208966 | HMGA1 |
| 2024 | Homo sapiens prefoldin 5 (PFDN5), transcript variant 1, mRNA | NM_002624 | 22202632 | PFDN5 |
| 2026 | Homo sapiens microsomal glutathione S-transferase 3 (MGST3), mRNA | NM_004528 | 22035640 | MGST3 |
| 2028 | Homo sapiens adaptor-related protein complex 1, beta 1 subunit (AP1B1), | NM_145730 | 22027652 | AP1B1 |
| | transcript variant 2, mRNA | | | |
| 2030 | Homo sapiens low density lipoprotein receptor adaptor protein 1 (LDLRAP1), | NM_015627 | 22027529 | LDLRAP1 |
| | mRNA | | | |
| 2031 | Homo sapiens cell division cycle associated 7 (CDCA7), transcript variant 2, | NM_145810 | 22027513 | CDCA7 |
| | mRNA | | | |
| 2032 | Homo sapiens myo-inositol 1-phosphate synthase A1 (ISYNA1), mRNA | NM_016368 | 21902536 | ISYNA1 |
| 2033 | Homo sapiens cDNA FLJ40890 fis, clone UTERU2001024, highly similar to | AK098209 | 21758176 | |
| | SPLICING FACTOR, ARGININE/SERINE-RICH 2 | | | |
| 2034 | Homo sapiens cDNA FLJ40776 fis, clone TRACH2005210 | AK098095 | 21758033 | |
| 2035 | Homo sapiens cDNA FLJ40363 fis, clone TESTI2034718, weakly similar to Mus | AK097682 | 21757530 | |
| | musculus membrane protein TMS-2 mRNA | | | |
| 2036 | Homo sapiens cDNA FLJ39736 fis, clone SMINT2016313, highly similar to G- | AK097055 | 21756699 | |
| | RICH SEQUENCE FACTOR-1 | | | |
| 2037 | Homo sapiens cDNA FLJ39640 fis, clone SMINT2003386, highly similar to DNA | AK096959 | 21756578 | |
| | REPLICATION LICENSING FACTOR MCM7 | | | |
| 2038 | Homo sapiens cDNA FLJ39507 fis, clone PROST2017617, highly similar to Homo | AK096826 | 21756403 | |
| | sapiens ENIGMA protein mRNA | | | |
| 2039 | Homo sapiens cDNA FLJ38612 fis, clone HEART2006195, weakly similar to Homo | AK095931 | 21755290 | |
| | sapiens mRNA for myopodin | | | |
| 2040 | Homo sapiens cDNA FLJ38560 fis, clone HCHON2003642, highly similar to Homo | AK095879 | 21755224 | |
| | sapiens mRNA for PED phosphoprotein | | | |
| 2041 | Homo sapiens cDNA FLJ38520 fis, clone HCHON2000781, weakly similar to | AK095839 | 21755181 | |
| | Cisplatin resistance-associated overexpressed protein | | | |
| 2042 | Homo sapiens cDNA FLJ38173 fis, clone FCBBF1000053, highly similar to | AK095492 | 21754757 | |
| | HYDROXYMETHYLGLUTARYL-COA SYNTHASE, CYTOPLASMIC (EC 4.1.3.5) | | | |
| 2043 | Homo sapiens cDNA FLJ38137 fis, clone D9OST2001433, highly similar to Homo | AK095456 | 21754712 | |
| | sapiens cellular repressor of E1A-stimulated genes CREG mRNA | | | |
| 2044 | Homo sapiens cDNA FLJ37260 fis, clone BRAMY2010441, highly similar to | AK094579 | 21753666 | |
| | PEANUT-LIKE PROTEIN 2 | | | |
| 2045 | Homo sapiens cDNA FLJ36793 fis, clone ADRGL2006124, highly similar to | AK094112 | 21753106 | |
| | SCG10-like-protein | | | |
| 2046 | Homo sapiens cDNA FLJ34577 fis, clone KIDNE2008403 | AK091896 | 21750369 | |
| 2047 | Homo sapiens malate dehydrogenase 1, NAD (soluble) (MDH1), mRNA | NM_005917 | 21735619 | MDH1 |
| 2048 | Homo sapiens programmed cell death 5 (PDCD5), mRNA | NM_004708 | 21735599 | PDCD5 |
| 2049 | Homo sapiens mRNA; cDNA DKFZp667K0520 (from clone DKFZp667K0520) | AL833651 | 21734299 | |
| 2050 | Homo sapiens mRNA; cDNA DKFZp667O202 (from clone DKFZp667O202) | AL832866 | 21733450 | |
| 2051 | Homo sapiens mRNA; cDNA DKFZp667P1024 (from clone DKFZp667P1024) | AL832835 | 21733418 | |
| 2052 | Homo sapiens mRNA; cDNA DKFZp686I1727 (from clone DKFZp686I1727) | AL832766 | 21733348 | |
| 2053 | Homo sapiens mRNA; cDNA DKFZp451C066 (from clone DKFZp451C066) | AL832353 | 21732915 | |
| 2054 | Homo sapiens solute carrier family 15, member 4 (SLC15A4), mRNA | NM_145648 | 21717815 | SLC15A4 |
| 2055 | Homo sapiens cortactin, mRNA (cDNA clone MGC: 33091 IMAGE: 4824206), | BC033889 | 21707901 | |
| | complete cds | | | |
| 2056 | Homo sapiens UBX domain containing 5 (UBXD5), transcript variant 1, mRNA | NM_145345 | 21703361 | UBXD5 |
| 2057 | Homo sapiens family with sequence similarity 14, member B (FAM14B), mRNA | NM_145249 | 21699069 | FAM14B |
| 2058 | Homo sapiens mitogen-activated protein kinase kinase 3 (MAP2K3), transcript | NM_002756 | 21618346 | MAP2K3 |
| | variant A, mRNA | | | |
| 2059 | Homo sapiens signal transducer and activator of transcription 5A (STAT5A), | NM_003152 | 21618341 | STAT5A |
| | mRNA | | | |
| 2060 | Homo sapiens testicular soluble adenylyl cyclase (SAC), mRNA | NM_018417 | 21614540 | SAC |
| 2061 | Homo sapiens glypican 4 (GPC4), mRNA | NM_001448 | 21614524 | GPC4 |
| 2062 | Homo sapiens aldehyde dehydrogenase 1 family, member L1 (ALDH1L1), mRNA | NM_012190 | 21614512 | ALDH1L1 |
| 2063 | Homo sapiens NAD(P) dependent steroid dehydrogenase-like (HSPC105), mRNA | NM_145168 | 21553310 | HSPC105 |
| 2064 | Homo sapiens AXL receptor tyrosine kinase (AXL), transcript variant 2, mRNA | NM_001699 | 21536467 | AXL |
| 2065 | Homo sapiens prefoldin 1 (PFDN1), mRNA | NM_002622 | 21536448 | PFDN1 |
| 2066 | Homo sapiens benzodiazapine receptor (peripheral) (BZRP), transcript variant | NM_000714 | 21536444 | BZRP |
| | PBR, mRNA | | | |
| 2067 | Homo sapiens tissue inhibitor of metalloproteinase 3 (Sorsby fundus dystrophy, | NM_000362 | 21536431 | TIMP3 |
| | pseudoinflammatory) (TIMP3), mRNA | | | |
| 2068 | Homo sapiens transferrin (TF), mRNA | NM_001063 | 21536430 | TF |
| 2069 | Homo sapiens amphiphysin (Stiff-Man syndrome with breast cancer 128 kDa | NM_001635 | 21536405 | AMPH |
| | autoantigen) (AMPH), transcript variant 1, mRNA | | | |
| 2070 | Homo sapiens ATP-binding cassette, sub-family B (MDR/TAP), member 11 | NM_003742 | 21536377 | MDR/TAP |
| | (ABCB11), mRNA | | | |
| 2071 | Homo sapiens DEAD (Asp-Glu-Ala-Asp) box polypeptide 6 (DDX6), mRNA | NM_004397 | 21536329 | DDX6 |
| 2072 | Homo sapiens suppressor of cytokine signaling 2 (SOCS2), mRNA | NM_003877 | 21536304 | SOCS2 |
| 2073 | Homo sapiens cAMP responsive element binding protein-like 2 (CREBL2), mRNA | NM_001310 | 21536277 | CREBL2 |
| 2075 | Homo sapiens methyl-CpG binding domain protein 2 (MBD2), transcript variant 1, | NM_003927 | 21464121 | MBD2 |
| | mRNA | | | |
| 2076 | Homo sapiens hypothetical protein FLJ23790 (FLJ23790), mRNA | NM_144963 | 21450642 | FLJ23790 |
| 2078 | Homo sapiens chromosome 1 open reading frame 58 (C1orf58), mRNA | NM_144695 | 21389600 | C1orf58 |
| 2079 | Homo sapiens zinc finger, matrin type 2 (ZMAT2), mRNA | NM_144723 | 21389510 | ZMAT2 |
| 2080 | Homo sapiens hypothetical protein FLJ32926 (FLJ32926), mRNA | NM_144577 | 21389346 | FLJ32926 |
| 2081 | Homo sapiens nexilin (F actin binding protein) (NEXN), mRNA | NM_144573 | 21389338 | NEXN |
| 2082 | Homo sapiens thioredoxin-like 5 (TXNL5), mRNA | NM_032731 | 21362103 | TXNL5 |
| 2083 | Homo sapiens ankyrin repeat, family A (RFXANK-like), 2 (ANKRA2), mRNA | NM_023039 | 21362082 | RFXANK- |
| | | | | like |
| 2084 | Homo sapiens hypothetical protein MGC13114 (MGC13114), mRNA | NM_032366 | 21362059 | MGC13114 |
| 2085 | Homo sapiens chromosome 9 open reading frame 89 (C9orf89), mRNA | NM_032310 | 21362043 | C9orf89 |
| 2086 | cand_Human_LPD_606_FL_26Jun2003.fa | NM_024647 | 21362033 | Nup43 |
| | BaseSame: gi\|21362033\|ref\|NM_024647.2\|Homo sapiens nucleoporin Nup43 | | | |
| | (Nup43) | | | |
| 2087 | Homo sapiens chromosome 19 open reading frame 27 (C19orf27), mRNA | NM_031213 | 21361948 | C19orf27 |
| 2088 | Homo sapiens tankyrase, TRF1-interacting ankyrin-related ADP-ribose polymerase | NM_025235 | 21361945 | TNKS2 |
| | 2 (TNKS2), mRNA | | | |
| 2089 | Homo sapiens FK506 binding protein 10, 65 kDa (FKBP10), mRNA | NM_021939 | 21361894 | FKBP10 |
| 2090 | Homo sapiens glutamate-ammonia ligase (glutamine synthase) (GLUL), mRNA | NM_002065 | 21361767 | GLUL |
| 2091 | Homo sapiens hypothetical protein FLJ10656 (P15RS), mRNA | NM_018170 | 21361708 | P15RS |
| 2092 | Homo sapiens COMM domain containing 4 (COMMD4), mRNA | NM_017828 | 21361660 | COMMD4 |
| 2093 | Homo sapiens RRN3 RNA polymerase I transcription factor homolog (yeast) | NM_018427 | 21361630 | RRN3 |
| | (RRN3), mRNA | | | |
| 2094 | Homo sapiens toll interacting protein (TOLLIP), mRNA | NM_019009 | 21361618 | TOLLIP |
| 2095 | Homo sapiens hypothetical protein FLJ20152 (FLJ20152), mRNA | NM_019000 | 21361616 | FLJ20152 |
| 2096 | Homo sapiens Huntingtin interacting protein K (HYPK), mRNA | NM_016400 | 21361540 | HYPK |
| 2097 | Homo sapiens hypothetical protein HSPC138 (HSPC138), mRNA | NM_016401 | 21361534 | HSPC138 |
| 2098 | Homo sapiens adiponectin receptor 1 (ADIPOR1), mRNA | NM_015999 | 21361518 | ADIPOR1 |
| 2099 | Homo sapiens neural precursor cell expressed, developmentally down-regulated 4- | NM_015277 | 21361471 | NEDD4L |
| | like (NEDD4L), mRNA | | | |
| 2100 | Homo sapiens glycogen synthase kinase 3 beta (GSK3B), mRNA | NM_002093 | 21361339 | GSK3B |
| 2101 | Homo sapiens tubulin, beta 4 (TUBB4), mRNA | NM_006087 | 21361321 | TUBB4 |
| 2102 | Homo sapiens retinol dehydrogenase 16 (all-trans and 13-cis) (RDH16), mRNA | NM_003708 | 21361104 | RDH16 |
| 2104 | Homo sapiens cold shock domain protein A (CSDA), mRNA | NM_003651 | 21359983 | CSDA |
| 2105 | Homo sapiens SECIS binding protein 2 (SECISBP2), mRNA | NM_024077 | 21359954 | SECISBP2 |
| 2106 | Homo sapiens Down syndrome cell adhesion molecule like 1 (DSCAML1), mRNA | NM_020693 | 21359934 | DSCAML1 |
| 2107 | Homo sapiens TGF beta-inducible nuclear protein 1 (TINP1), mRNA | NM_014886 | 21359901 | TINP1 |
| 2108 | Homo sapiens makorin, ring finger protein, 1 (MKRN1), mRNA | NM_013446 | 21359891 | MKRN1 |
| 2109 | Homo sapiens angiopoietin 1 (ANGPT1), mRNA | NM_001146 | 21328452 | ANGPT1 |
| 2111 | Homo sapiens coated vesicle membrane protein (RNP24), mRNA | NM_006815 | 21314646 | RNP24 |
| 2112 | Homo sapiens CD4 antigen (p55) (CD4), mRNA | NM_000616 | 21314613 | CD4 |
| 2115 | Homo sapiens signal transducing adaptor molecule (SH3 domain and ITAM motif) | NM_005843 | 21265030 | STAM2 |
| | 2 (STAM2), mRNA | | | |
| 2116 | Homo sapiens lysyl oxidase (LOX), mRNA | NM_002317 | 21264603 | LOX |
| 2117 | Homo sapiens spermine synthase (SMS), mRNA | NM_004595 | 21264340 | SMS |
| 2118 | Homo sapiens leucine rich repeat containing 8 family, member B (LRRC8B), | NM_015350 | 21245133 | LRRC8B |
| | mRNA | | | |
| 2119 | Homo sapiens SWI/SNF related, matrix associated, actin dependent regulator of | NM_003074 | 21237801 | SMARCC1 |
| | chromatin, subfamily c, member 1 (SMARCC1), mRNA | | | |
| 2120 | Homo sapiens CD9 antigen (p24) (CD9), mRNA | NM_001769 | 21237762 | CD9 |
| 2121 | Homo sapiens component of oligomeric golgi complex 8 (COG8), mRNA | NM_032382 | 21166360 | COG8 |
| 2122 | Homo sapiens peptidylglycine alpha-amidating monooxygenase (PAM), transcript | NM_138822 | 21070981 | PAM |
| | variant 4, mRNA | | | |
| 2123 | Homo sapiens elongation factor-2 kinase mRNA, complete cds | U93850 | 2104698 | EEF2K |
| 2124 | Homo sapiens DEAD (Asp-Glu-Ala-Asp) box polypeptide 39 (DDX39), transcript | NM_005804 | 21040370 | DDX39 |
| | variant 1, mRNA | | | |
| 2125 | Homo sapiens B-cell CLL/lymphoma 6 (zinc finger protein 51) (BCL6), transcript | NM_001706 | 21040323 | BCL6 |
| | variant 1, mRNA | | | |
| 2126 | Homo sapiens mitogen-activated protein kinase 1 (MAPK1), transcript variant 2, | NM_138957 | 20986530 | MAPK1 |
| | mRNA | | | |
| 2127 | Homo sapiens mitogen-activated protein kinase 14 (MAPK14), transcript variant 2, | NM_139012 | 20986511 | MAPK14 |
| | mRNA | | | |
| 2128 | Human huntingtin interacting protein (HIP1) mRNA, complete cds | U79734 | 2072422 | HIP1 |
| 2129 | Homo sapiens cAMP responsive element binding protein-like 1 (CREBL1), mRNA | NM_004381 | 20631976 | CREBL1 |
| 2130 | Homo sapiens casein kinase 1, delta (CSNK1D), transcript variant 2, mRNA | NM_139062 | 20544144 | CSNK1D |
| 2131 | Homo sapiens casein kinase 1, delta (CSNK1D), transcript variant 1, mRNA | NM_001893 | 20544143 | CSNK1D |
| 2132 | Homo sapiens mRNA for KIAA1412 protein, partial cds | AB037833 | 20521903 | |
| 2133 | Homo sapiens mRNA for KIAA1096 protein, partial cds | AB029019 | 20521763 | |
| 2134 | Homo sapiens mRNA for KIAA0864 protein, partial cds | AB020671 | 20521679 | |
| 2135 | Homo sapiens clusterin (complement lysis inhibitor, SP-40,40, sulfated | BC029972 | 20455818 | |
| | glycoprotein 2, testosterone-repressed prostate message 2, apolipoprotein J), | | | |
| | mRNA (cDNA clone IMAGE: 4939961) | | | |
| 2136 | Homo sapiens ATPase, H+ transporting, lysosomal 14 kDa, V1 subunit F | NM_004231 | 20357546 | ATP6V1F |
| | (ATP6V1F), mRNA | | | |
| 2137 | Homo sapiens guanine nucleotide binding protein (G protein), beta polypeptide 2 | NM_005273 | 20357528 | GNB2 |
| | (GNB2), mRNA | | | |
| 2138 | Homo sapiens uridine-cytidine kinase 2 (UCK2), mRNA | NM_012474 | 20357519 | UCK2 |
| 2139 | Homo sapiens B-cell CLL/lymphoma 10 (BCL10), mRNA | NM_003921 | 20336470 | BCL10 |
| 2142 | Homo sapiens A kinase (PRKA) anchor protein 7 (AKAP7), transcript variant | NM_004842 | 20336197 | PRKA |
| | alpha, mRNA | | | |
| 2143 | Homo sapiens phosphatidylinositol-4-phosphate 5-kinase, type II, alpha | NM_005028 | 20302162 | PIP5K2A |
| | (PIP5K2A), mRNA | | | |
| 2144 | Homo sapiens lysophospholipase II (LYPLA2), mRNA | NM_007260 | 20302149 | LYPLA2 |
| 2145 | Homo sapiens tankyrase 1 binding protein 1, 182 kDa (TNKS1BP1), mRNA | NM_033396 | 20270211 | TNKS1BP1 |
| 2146 | Homo sapiens neural proliferation, differentiation and control, 1 (NPDC1), mRNA | NM_015392 | 20149616 | NPDC1 |
| 2147 | Homo sapiens heat shock 90 kDa protein 1, beta (HSPCB), mRNA | NM_007355 | 20149593 | HSPCB |
| 2148 | Homo sapiens serine/threonine kinase receptor associated protein (STRAP), | NM_007178 | 20149591 | STRAP |
| | mRNA | | | |
| 2149 | Homo sapiens nuclear factor (erythroid-derived 2)-like 2 (NFE2L2), mRNA | NM_006164 | 20149575 | NFE2L2 |
| 2150 | Homo sapiens integrin beta 1 binding protein 1 (ITGB1BP1), transcript variant 1, | NM_004763 | 20143950 | ITGB1BP1 |
| | mRNA | | | |
| 2151 | Homo sapiens tumor protein p53 inducible nuclear protein 1 (TP53INP1), mRNA | NM_033285 | 20127661 | TP53INP1 |
| 2152 | Homo sapiens angiopoietin-like 7 (ANGPTL7), mRNA | NM_021146 | 20127595 | ANGPTL7 |
| 2153 | Homo sapiens PDZ and LIM domain 1 (elfin) (PDLIM1), mRNA | NM_020992 | 20127594 | PDLIM1 |
| 2154 | Homo sapiens hypothetical protein HSPC111 (HSPC111), mRNA | NM_016391 | 20127554 | HSPC111 |
| 2155 | Homo sapiens poly (ADP-ribose) polymerase family, member 6, mRNA (cDNA | BC026955 | 20073232 | |
| | clone IMAGE: 5106605) | | | |
| 2156 | Homo sapiens transmembrane protein 49 (TMEM49), mRNA | NM_030938 | 20070348 | TMEM49 |
| 2157 | Homo sapiens docking protein 4 (DOK4), mRNA | NM_018110 | 20070301 | DOK4 |
| 2158 | Homo sapiens tRNA nucleotidyl transferase, CCA-adding, 1 (TRNT1), transcript | NM_016000 | 20070270 | TRNT1 |
| | varient 1, mRNA | | | |
| 2159 | Homo sapiens ubiquinol-cytochrome c reductase binding protein (UQCRB), mRNA | NM_006294 | 20070231 | UQCRB |
| 2160 | Homo sapiens Ras homolog enriched in brain (RHEB), mRNA | NM_005614 | 20070207 | RHEB |
| 2161 | Homo sapiens eukaryotic translation initiation factor 4E binding protein 1 | NM_004095 | 20070179 | EIF4EBP1 |
| | (EIF4EBP1), mRNA | | | |
| 2162 | Homo sapiens procollagen-proline, 2-oxoglutarate 4-dioxygenase (proline 4- | NM_000918 | 20070124 | P4HB |
| | hydroxylase), beta polypeptide (protein disulfide isomerase-associated 1) (P4HB), | | | |
| | mRNA | | | |
| 2163 | Homo sapiens suppressor of Ty 16 homolog (S. cerevisiae) (SUPT16H), mRNA | NM_007192 | 19924176 | SUPT16H |
| 2164 | Homo sapiens RAD9 homolog A (S. pombe) (RAD9A), mRNA | NM_004584 | 19924112 | RAD9A |
| 2165 | Homo sapiens chromosome 7 open reading frame 30 (C7orf30), mRNA | NM_138446 | 19923976 | C7orf30 |
| 2166 | Homo sapiens hypothetical transmembrane protein SBBI54 (SBBI54), mRNA | NM_138334 | 19923878 | SBBI54 |
| 2167 | Homo sapiens pre-B-cell leukemia transcription factor interacting protein 1 | NM_020524 | 19923829 | PBXIP1 |
| | (PBXIP1), mRNA | | | |
| 2168 | Homo sapiens solute carrier family 16 (monocarboxylic acid transporters), member | NM_003051 | 19923752 | SLC16A1 |
| | 1 (SLC16A1), mRNA | | | |
| 2169 | Homo sapiens GATA zinc finger domain containing 2A (GATAD2A), mRNA | NM_017660 | 19923501 | GATAD2A |
| 2170 | Homo sapiens serine/arginine repetitive matrix 2 (SRRM2), mRNA | NM_016333 | 19923465 | SRRM2 |
| 2171 | Homo sapiens adenylate kinase 3 (AK3), mRNA | NM_016282 | 19923436 | AK3 |
| 2172 | Homo sapiens sodium channel, voltage-gated, type III, alpha (SCN3A), mRNA | NM_006922 | 19923380 | SCN3A |
| 2173 | Homo sapiens RAB4A, member RAS oncogene family (RAB4A), mRNA | NM_004578 | 19923259 | RAB4A |
| 2174 | Homo sapiens cytoskeleton-associated protein 4 (CKAP4), mRNA | NM_006825 | 19920316 | CKAP4 |
| 2175 | Homo sapiens ATPase, H+ transporting, lysosomal 31 kDa, V1 subunit E isoform 1 | NM_001696 | 19913433 | ATP6V1E1 |
| | (ATP3V1E1), mRNA | | | |
| 2176 | Homo sapiens ATPase, H+ transporting, lysosomal 56/58 kDa, V1 subunit B, | NM_001693 | 19913427 | ATP6V1B2 |
| | isoform 2 (ATP6V1B2), mRNA | | | |
| 2177 | Homo sapiens major vault protein (MVP), transcript variant 2, mRNA | NM_005115 | 19913411 | MVP |
| 2179 | Homo sapiens surfeit 4 (SURF4), mRNA | NM_033161 | 19593984 | SURF4 |
| 2180 | Homo sapiens bone morphogenetic protein 4 (BMP4), transcript variant 1, mRNA | NM_001202 | 19528648 | BMP4 |
| 2181 | Homo sapiens N-acylsphingosine amidohydrolase (alkaline ceramidase) 3 | NM_133492 | 19424127 | ASAH3 |
| | (ASAH3), mRNA | | | |
| 2182 | Homo sapiens, Tax1 (human T-cell leukemia virus type I) binding protein 1, clone | BC024600 | 19354206 | |
| | IMAGE: 4339438, mRNA | | | |
| 2183 | Homo sapiens TPMsk3 (TPM3) mRNA, partial cds | AF474157 | 19072648 | TPM3 |
| 2184 | cr: gi\|189503\|gb\|M62399.1\|HUMP65NFKB Human NF-kappa-B transcription factor | M62399 | 189503 | |
| | p65 DNA binding subunit mRNA, complete cds | | | |
| 2185 | Human major nuclear matrix protein mRNA | M63483 | 188622 | |
| 2186 | Homo sapiens protein tyrosine phosphatase, receptor type, D (PTPRD), transcript | NM_130393 | 18860893 | PTPRD |
| | vriant 4, mRNA | | | |
| 2187 | Homo sapiens cDNA clone IMAGE: 3508235, containing frame-shift errors | BC024102 | 18848186 | |
| 2188 | Homo sapiens collagen, type XVIII, alpha 1 (COL18A1), transcript variant 1, mRNA | NM_030582 | 18765744 | COL18A1 |
| 2189 | Homo sapiens tumor susceptibility gene 101 (TSG101), mRNA | NM_006292 | 18765712 | TSG101 |
| 2190 | Homo sapiens collagen, type XV, alpha 1 (COL15A1), mRNA | NM_001855 | 18641349 | COL15A1 |
| 2191 | Homo sapiens WNT1 inducible signaling pathway protein 2 (WISP2), mRNA | NM_003881 | 18491001 | WISP2 |
| 2193 | Homo sapiens GNAS complex locus (GNAS), transcript variant 2, mRNA | NM_080426 | 18426899 | GNAS |
| 2194 | Homo sapiens GNAS complex locus (GNAS), transcript variant 2, mRNA | NM_000516 | 18426896 | GNAS |
| 2195 | Homo sapiens sortilin-related VPS10 domain containing receptor 3 (SORCS3), | NM_014978 | 18379345 | SORCS3 |
| | mRNA | | | |
| 2196 | Homo sapiens RNA binding protein S1, serine-rich domain (RNPS1), transcript | NM_080594 | 18379333 | RNPS1 |
| | variant 2, mRNA | | | |
| 2197 | Homo sapiens centrosome-associated protein 350 (CAP350), mRNA | NM_014810 | 18378734 | CAP350 |
| 2198 | Homo sapiens protein tyrosine phosphatase, non-receptor type 18 (brain-derived) | NM_014369 | 18375654 | PTPN18 |
| | (PTPN18), mRNA | | | |
| 2199 | Homo sapiens collagen, type XII, alpha 1 (COL12A1), transcript variant short, | NM_080645 | 18201920 | COL12A1 |
| | mRNA | | | |
| 2200 | Homo sapiens vitronectin (serum spreading factor, somatomedin B, complement | NM_000638 | 18201910 | VTN |
| | S-protein) (VTN), mRNA | | | |
| 2201 | Homo sapiens mRNA for translocation protein-1, complete cds | D87127 | 1817551 | |
| 2202 | Homo sapiens egl nine homolog 2 (C. elegans) (EGLN2), transcript variant 1, | NM_053046 | 18141576 | EGLN2 |
| | mRNA | | | |
| 2203 | Homo sapiens DNA for cyclin G, partial cds | D86077 | 1813373 | |
| 2208 | Homo sapiens cytochrome c oxidase subunit VIIa polypeptide 2 (liver) (COX7A2), | NM_001865 | 18105035 | COX7A2 |
| | mRNA | | | |
| 2209 | Homo sapiens protein tyrasine phosphatase type IVA, member 2 (PTP4A2), | NM_003479 | 18104974 | PTP4A2 |
| | transcript variant 1, mRNA | | | |
| 2210 | Homo sapiens prostaglandin-endaperoxide synthase 1 (prostaglandin G/H | NM_000962 | 18104966 | PTGS1 |
| | synthase and cyclooxygenase) (PTGS1), transcript variant 1, mRNA | | | |
| 2211 | Homo sapiens amyloid beta precursor protein (cytoplasmic tall) binding protein 2 | NM_006380 | 18104961 | APPBP2 |
| | (APPBP2), mRNA | | | |
| 2212 | Homo sapiens similar to RIKEN cDNA 5730528L13 gene (MGC17337), mRNA | NM_080655 | 18087818 | MGC17337 |
| 2213 | Homo sapiens G protein beta1/gamma2 subunit-interacting factor 3, partial | AF456479 | 18087417 | |
| | sequence | | | |
| 2214 | Homo sapiens cytochrome c oxidase subunit VIc (COX6C), mRNA | NM_004374 | 17999531 | COX6C |
| 2215 | Homo sapiens cytochrome c oxidase subunit Vib polypeptide 1 (ubiquitous) | NM_001863 | 17999530 | COX6B1 |
| | (COX6B1), mRNA | | | |
| 2217 | Homo sapiens myosin light chain 1 slow a (MLC1SA), mRNA | NM_002475 | 17986280 | MLC1SA |
| 2218 | Homo sapiens collagen, type IV, alpha 2 (COL4A2), mRNA | NM_001846 | 17986276 | COL4A2 |
| 2219 | Homo sapiens myosin, light polypeptide 6, alkali, smooth muscle and non-muscle | NM_021019 | 17986257 | MYL6 |
| | (MYL6), transcript variant 1, mRNA | | | |
| 2220 | Homo sapiens septin 4 (SEPT4), transcript variant 3, mRNA | NM_080416 | 17986246 | SEPT4 |
| 2221 | Homo sapiens cyclin-dependent kinase inhibitor 1B (p27, Kip1) (CDKN1B), mRNA | NM_004064 | 17978497 | CDKN1B |
| 2222 | Homo sapiens cyclin-dependent kinase inhibitor 1A (p21, Cip1) (CDKN1A), | NM_078467 | 17978494 | CDKN1A |
| | transcript variant 2, mRNA | | | |
| 2223 | Homo sapiens CDK2-associated protein 1 (CDK2AP1), mRNA | NM_004642 | 17978492 | CDK2AP1 |
| 2224 | Homo sapiens GATA zinc finger domain containing 1, mRNA (cDNA clone | BC019350 | 17939643 | |
| | MGC: 13048 IMAGE: 3626609), complete cds | | | |
| 2225 | Homo sapiens tubulin, alpha 1 (testis specific) (TUBA1), mRNA | NM_006000 | 17921988 | TUBA1 |
| 2226 | Homo sapiens vacuolar protein sorting 4A (yeast) (VPS4A), mRNA | NM_013245 | 17865806 | VPS4A |
| 2227 | Homo sapiens BBP-like protein 2 (BLP2), transcript variant 1, mRNA | NM_078474 | 17865799 | BLP2 |
| 2228 | Homo sapiens wingless-type MMTV integration site family, member 7B (WNT7B), | NM_058238 | 17505192 | WNT7B |
| | mRNA | | | |
| 2229 | Homo sapiens vacuolar protein sorting 29 (yeast) (VPS29), transcript variant 2, | NM_057180 | 17402911 | VPS29 |
| | mRNA | | | |
| 2230 | Homo sapiens collagen, type VI, alpha 2 (COL6A2), transcript variant 2C2, mRNA | NM_001849 | 17402874 | COL6A2 |
| 2231 | Homo sapiens, Similar to transforming growth factor beta regulated gene 1, clone | BC018452 | 17391060 | |
| | IMAGE: 4500659, mRNA | | | |
| 2232 | Homo sapiens, clone IMAGE: 4668773, mRNA | BC017820 | 17389580 | |
| 2233 | Homo sapiens cyclin E1 (CCNE1), transcript variant 1, mRNA | NM_001238 | 17318558 | CCNE1 |
| 2234 | Homo sapiens FK506 binding protein 5 (FKBP5), mRNA | NM_004117 | 17149847 | FKBP5 |
| 2235 | Homo sapiens FK506 binding protein 3, 25 kDa (FKBP3), mRNA | NM_002013 | 17149845 | FKBP3 |
| 2236 | Homo sapiens FK506 binding protein 2, 13 kDa (FKBP2), transcript variant 2, | NM_057092 | 17149843 | FKBP2 |
| | mRNA | | | |
| 2237 | Homo sapiens FK506 binding protein 2, 13 kDa (FKBP2), transcript variant 1, | NM_004470 | 17149841 | FKBP2 |
| | mRNA | | | |
| 2238 | Homo sapiens FK506 binding protein 1A, 12 kDa (FKBP1A), transcript variant 12B, | NM_000801 | 17149837 | FKBP1A |
| | mRNA | | | |
| 2239 | Homo sapiens kelch domain containing 3 (KLHDC3), mRNA | NM_057161 | 17149812 | KLHDC3 |
| 2241 | Homo sapiens Kell blood group (KEL), mRNA | NM_000420 | 17025233 | KEL |
| 2245 | Homo sapiens cyclin D1 (PRAD1: parathyroid edenomatosis 1) (CCND1), mRNA | NM_053056 | 16950654 | CCND1 |
| 2246 | Homo sapiens cyclin-dependent kinase 4 (CDK4), mRNA | NM_000075 | 16936531 | CDK4 |
| 2247 | Homo sapiens cyclin-dependent kinase 2 (CDK2), transcript variant 1, mRNA | NM_001798 | 16936527 | CDK2 |
| 2249 | Homo sapiens talin 1 (TLN1), mRNA | NM_006289 | 16753232 | TLN1 |
| 2251 | Homo sapiens mRNA for KIAA0277 gene, partial cds | D87467 | 1665818 | |
| 2252 | Homo sapiens mRNA for KIAA0273 gene, partial cds | D87463 | 1665810 | |
| 2253 | Homo sapiens fructose-1,6-bisphosphatase 1 (FBP1), mRNA | NM_000507 | 16579887 | FBP1 |
| 2255 | Homo sapiens immediate early response 3 (IER3), transcript variant long, mRNA | NM_052815 | 16554596 | IER3 |
| 2256 | Homo sapiens immediate early response 3 (IER3), transcript variant short, mRNA | NM_003897 | 16554595 | IER3 |
| 2257 | Homo sapiens collagen, type V, alpha 2 (COL5A2), mRNA | NM_000393 | 16554580 | COL5A2 |
| 2258 | Homo sapiens collagen, type V, alpha 1 (COL5A1), mRNA | NM_000093 | 16554578 | COL5A1 |
| 2259 | Homo sapiens cDNA FLJ32947 fis, clone TESTI2007951, highly similar to | AK057509 | 16553243 | |
| | NEURAL CELL ADHESION MOLECULE, 140 KDA ISOFORM PRECURSOR | | | |
| 2260 | Homo sapiens cDNA FLJ32730 fis, clone TESTI2001099, highly similar to Homo | AK057292 | 16552923 | |
| | sapiens homolog of the Aspergilius nidulans sudD gene product mRNA | | | |
| 2261 | Homo sapiens cDNA FLJ32068 fis, clone OCBBF1000114 | AK056630 | 16552085 | |
| 2262 | Homo sapiens cDNA FLJ31433 fis, clone NT2NE2000575, highly similar to | AK055995 | 16550865 | |
| | PROBABLE RNA-DEPENDENT HELICASE P68 | | | |
| 2263 | Homo sapiens cDNA FLJ31392 fis, clone NT2NE1000073, weakly similar to RAB | AK055954 | 16550811 | |
| | GERANYLGERANYLTRANSFERASE ALPHA SUBUNIT (EC 2.5.1.—) | | | |
| 2264 | Homo sapiens cDNA FLJ30515 fis, clone BRAWH2000686, highly similar to | AK055077 | 16549730 | |
| | SERINE/THREONINE-PROTEIN KINASE SGK (EC 2.7.1.—) | | | |
| 2265 | Homo sapiens cDNA FLJ30017 fis, clone 3NB692000484, highly similar to | AK054579 | 16549145 | |
| | UBIQUITIN CARBOXYL-TERMINAL HYDROLASE ISOZYME L1 (EC 3.1.2.15) | | | |
| 2266 | Homo sapiens eukaryotic translation elongation factor 1 beta 2 (EEF1B2), | NM_001959 | 16519564 | EEF1B2 |
| | transcript variant 1, mRNA | | | |
| 2268 | Homo sapiens cadherin 15, M-cadherin (myotubule) (CDH15), mRNA | NM_004933 | 16507957 | CDH15 |
| 2270 | Human mRNA for TPRDI, complete cds | D84294 | 1632761 | |
| 2271 | Homo sapiens mitogen-activated protein kinase 6 (MAPK6), mRNA | NM_002748 | 16306569 | MAPK6 |
| 2275 | Homo sapiens selenium binding protein 1 (SELENBP1), mRNA | NM_003944 | 16306549 | SELENBP1 |
| 2276 | Homo sapiens cell division cycle 2, G1 to S and G2 to M (CDC2), transcript variant | NM_001786 | 16306490 | CDC2 |
| | 1, mRNA | | | |
| 2277 | Homo sapiens serine/threanine kinase TAO1 mRNA, complete cds | AY049015 | 16118410 | |
| 2282 | cr: gi\|16033442\|gb\|AF385323.1\|AF385323 Homo sapiens MDM2 variant FB26 | AF385323 | 16033442 | MDM2 |
| | (MDM2) mRNA, complete cds, alternatively spliced | | | |
| 2283 | Homo sapiens RNA binding motif protein 8A (RBM8A), mRNA | NM_005105 | 15812217 | RBM8A |
| 2284 | Homo sapiens F-box protein 7 (FBXO7), mRNA | NM_012179 | 15812192 | FBXO7 |
| 2285 | Homo sapiens zinc finger protein 36, C3H type-like 1 (ZFP36L1), mRNA | NM_004926 | 15812179 | ZFP36L1 |
| 2286 | Homo sapiens DiGeorge syndrome critical region gene 6-like (DGCR6L), mRNA | NM_033257 | 15718677 | DGCR6L |
| 2287 | Homo sapiens adrenergic, beta-2-, receptor, surface (ADRB2), mRNA | NM_000024 | 15718673 | ADRB2 |
| 2288 | Homo sapiens sorting nexin SNX22 (SNX22) mRNA, complete cds | AY044653 | 15554299 | SNX22 |
| 2290 | Homo sapiens sparc/osteonectin, cwcv and kazal-like domains proteoglycan | NM_004598 | 15451924 | SPOCK |
| | (testican) (SPOCK), mRNA | | | |
| 2291 | Homo sapiens eukaryotic translation initiation factor 5B (EIF5B), mRNA | NM_015904 | 15451891 | EIF5B |
| 2294 | Homo sapiens tripartite motif-containing 65, mRNA (cDNA clone IMAGE: 4385873), | BC013181 | 15341970 | |
| | complete cds | | | |
| 2295 | Homo sapiens collagen, type III, alpha 1 (Ehlers-Danlos syndrome type IV, | NM_000090 | 15149480 | COL3A1 |
| | autosomal dominant) (COL3A1), mRNA | | | |
| 2296 | Homo sapiens tripartite motif-containing 28 (TRIM28), mRNA | NM_005762 | 14971416 | TRIM28 |
| 2297 | Homo sapiens signal recognition particle receptor, B subunit (SRPRB), mRNA | NM_021203 | 14917112 | SRPRB |
| 2298 | Homo sapiens spen homolog, transcriptional regulator (Drosophila) (SPEN), | NM_015001 | 14790189 | SPEN |
| | mRNA | | | |
| 2299 | Homo sapiens caspase 9, apoptosis-related cysteine peptidase (CASP9), | NM_001229 | 14790123 | CASP9 |
| | transcript variant alpha, mRNA | | | |
| 2300 | Homo sapiens collagen, type I, alpha 1 (COL1A1), mRNA | NM_000088 | 14719826 | COL1A1 |
| 2301 | Homo sapiens NADH dehydrogenase (ubiquinone) 1 beta subcomplex, 11, | BC010665 | 14715010 | ubiquinone |
| | 17.3 kDa, mRNA (cDNA clone MGC: 8855 IMAGE: 3870660), complete cds | | | |
| 2302 | Homo sapiens, Similar to bromodomain-containing 2, clone IMAGE: 3544244, | BC001885 | 14706221 | |
| | mRNA | | | |
| 2303 | Homo sapiens BCL2-antagonist of cell death (BAD), transcript variant 1, mRNA | NM_004322 | 14670386 | BAD |
| 2304 | Homo sapiens poly(rC) binding protein 4 (PCBP4), transcript variant 3, mRNA | NM_033008 | 14670368 | PCBP4 |
| 2307 | Homo sapiens cadherin 5, type 2, VE-cadherin (vascular epithelium) (CDH5), | NM_001795 | 14589894 | CDH5 |
| | mRNA | | | |
| 2308 | Homo sapiens guanine nucleatide binding protein (G protein), gamma transducing | NM_031498 | 14589848 | GNGT2 |
| | activity polypeptide 2 (GNGT2), mRNA | | | |
| 2309 | Homo sapiens BCL2-related protein A1 (BCL2A1), mRNA | NM_004049 | 14574570 | BCL2A1 |
| 2310 | Homo sapiens nasopharyngeal carcinoma-related mRNA sequence | AF315788 | 14423106 | |
| 2311 | Homo sapiens protocadherin gamma subfamily C, 5 (PCDHGC5), transcript | NM_018929 | 14277683 | PCDHGC5 |
| | variant 1, mRNA | | | |
| 2312 | Homo sapiens protocadherin gamma subfamily C, 4 (PCDHGC4), transcript | NM_018928 | 14277680 | PCDHGC4 |
| | variant 1, mRNA | | | |
| 2313 | Homo sapiens protocadherin gamma subfamily C, 3 (PCDHGC3), transcript | NM_002588 | 14277674 | PCDHGC3 |
| | variant 1, mRNA | | | |
| 2314 | Homo sapiens protocadherin gamma subfamily B, 7 (PCDHGB7), transcript variant | NM_018927 | 14270506 | PCDHGB7 |
| | 1, mRNA | | | |
| 2315 | Homo sapiens protocadherin gamma subfamily B, 5 (PCDHGB5), transcript variant | NM_032099 | 14270504 | PCDHGB5 |
| | 2, mRNA | | | |
| 2316 | Homo sapiens protocadherin gamma subfamily B, 4 (PCDHGB4), transcript variant | NM_032098 | 14270498 | PCDHGB4 |
| | 2, mRNA | | | |
| 2317 | Homo sapiens protocadherin gamma subfamily B, 2 (PCDHGB2), transcript variant | NM_018923 | 14270491 | PCDHGB2 |
| | 1, mRNA | | | |
| 2318 | Homo sapiens protocadherin gamma subfamily B, 1 (PCDHGB1), transcript variant | NM_032095 | 14270489 | PCDHGB1 |
| | 2, mRNA | | | |
| 2319 | Homo sapiens protocadherin gamma subfamily A, B (PCDHGA8), transcript variant | NM_014004 | 14270482 | PCDHGA8 |
| | 2, mRNA | | | |
| 2320 | Homo sapiens hypothetical protein FLJ14825 (FLJ14825), mRNA | NM_032847 | 14249567 | FLJ14825 |
| 2321 | Homo sapiens hypothetical protein FLJ14525 (FLJ14525), mRNA | NM_032800 | 14249477 | FLJ14525 |
| 2322 | Homo sapiens Mov10, Moloney leukemia virus 10, homolog (mouse) (MOV10), | NM_020963 | 14211539 | MOV10 |
| | mRNA | | | |
| 2323 | Homo sapiens protocadherin gamma subfamily A, 7 (PCDHGA7), transcript variant | NM_032087 | 14196476 | PCDHGA7 |
| | 2, mRNA | | | |
| 2324 | Homo sapiens protocadherin gamma subfamily A, 5 (PCDHGA5), transcript variant | NM_018918 | 14196469 | PCDHGA5 |
| | 1, mRNA | | | |
| 2325 | Homo sapiens protocadherin gamma subfamily A, 4 (PCDHGA4), transcript variant | NM_018917 | 14196466 | PCDHGA4 |
| | 1, mRNA | | | |
| 2326 | Homo sapiens protocadherin gamma subfamily A, 3 (PCDHGA3), transcript variant | NM_032011 | 14196464 | PCDHGA3 |
| | 2, mRNA | | | |
| 2327 | Homo sapiens protocadherin gamma subfamily A, 2 (PCDHGA2), transcript variant | NM_018915 | 14196460 | PCDHGA2 |
| | 1, mRNA | | | |
| 2328 | Homo sapiens protocadherin gamma subfamily A, 1 (PCDHGA1), transcript variant | NM_018912 | 14196453 | PCDHGA1 |
| | 1, mRNA | | | |
| 2329 | Homo sapiens protocadherin gamma subfamily A, 12 (PCDHGA12), transcript | NM_003735 | 14196452 | PCDHGA12 |
| | variant 1, mRNA | | | |
| 2330 | Homo sapiens protocadherin gamma subfamily A, 10 (PCDHGA10), transcript | NM_032090 | 14196447 | PCDHGA10 |
| | variant 2, mRNA | | | |
| 2331 | Homo sapiens microtubule-associated protein 1B (MAP1B), transcript variant 1, | NM_005909 | 14165457 | MAP1B |
| | mRNA | | | |
| 2332 | Homo sapiens heterogeneous nuclear ribonucleoprotein K (HNRPK), transcript | NM_031262 | 14165434 | HNRPK |
| | variant 2, mRNA | | | |
| 2333 | Homo sapiens XPA binding protein 1, GTPase (XAB1), mRNA | NM_007266 | 14149628 | XAB1 |
| 2334 | Homo sapiens stromal cell-derived factor 2 (SDF2), mRNA | NM_006923 | 14141194 | SDF2 |
| 2336 | Homo sapiens fatty acid desaturase 2 (FADS2), mRNA | NM_004265 | 14141180 | FADS2 |
| 2337 | Homo sapiens high-mobility group box 2 (HMGB2), mRNA | NM_002129 | 14141173 | HMGB2 |
| 2338 | Homo sapiens poly(rC) binding protein 1 (PCBP1), mRNA | NM_006196 | 14141164 | PCBP1 |
| 2339 | Homo sapiens heterogeneous nuclear ribonucleoprotein U (scaffold attachment | NM_004501 | 14141160 | HNRPU |
| | factor A) (HNRPU), transcript variant 2, mRNA | | | |
| 2340 | Homo sapiens heterogeneous nuclear ribonucleoprotein M (HNRPM), transcript | NM_031203 | 14141153 | HNRPM |
| | variant 2, mRNA | | | |
| 2341 | Homo sapiens heterogeneous nuclear ribonucleoprotein M (HNRPM), transcript | NM_005968 | 14141151 | HNRPM |
| | variant 1, mRNA | | | |
| 2342 | Homo sapiens heterogeneous nuclear ribonucleoprotein C (C1/C2) (HNRPC), | NM_004500 | 14110430 | HNRPC |
| | transcript variant 2, mRNA | | | |
| 2343 | Homo sapiens cell division cycle 2-like 5 (cholinesterase-related cell division | NM_031267 | 14110389 | CDC2L5 |
| | controller) (CDC2L5), transcript variant 2, mRNA | | | |
| 2344 | Homo sapiens cell division cycle 2-like 5 (cholinesterase-related cell division | NM_003718 | 14110386 | CDC2L5 |
| | controller) (CDC2L5), transcript variant 1, mRNA | | | |
| 2345 | Homo sapiens heterogeneous nuclear ribonucleoprotein A2/B1 (HNRPA2B1), | NM_002137 | 14043073 | HNRPA2B1 |
| | transcript variant A2, mRNA | | | |
| 2346 | Homo sapiens heterogeneous nuclear ribonucleoprotein A2/B1 (HNRPA2B1), | NM_031243 | 14043071 | HNRPA2B1 |
| | transcript variant B1, mRNA | | | |
| 2347 | Homo sapiens hemoglobin, alpha 2 (HBA2), mRNA | NM_000517 | 14043068 | HBA2 |
| 2348 | Homo sapiens vesicle-associated membrane protein 8 (endobrevin) (VAMP8), | NM_003761 | 14043025 | VAMP8 |
| | mRNA | | | |
| 2349 | cr: gi\|14041994\|dbj\|AK027366.1\|Homo sapiens cDNA FLJ14460 fis, clone | AK027366 | 14041994 | |
| | HEMBB1002600, highly similar to Homo sapiens tetraspan NET-5 mRNA | | | |
| 2350 | Homo sapiens alkaline phosphatase, liver/bone/kidney (ALPL), mRNA | NM_000478 | 13787192 | ALPL |
| 2351 | Homo sapiens sirtuin (silent mating type information regulatian 2 homolog) 2 (S. cerevisiae) | NM_030593 | 13775601 | SIRT2 |
| | (SIRT2), transcript variant 2, mRNA | | | |
| 2352 | Homo sapiens methylenetetrahydrofolate dehydrogenase (NADP+ dependent) 1, | NM_005956 | 13699867 | MTHFD1 |
| | methenyltetrahydrofolate cyclohydrolase, formyltetrahydrofolate synthetase | | | |
| | (MTHFD1), mRNA | | | |
| 2353 | Homo sapiens hypoxia up-regulated 1 (HYOU1), mRNA | NM_006389 | 13699861 | HYOU1 |
| 2355 | Homo sapiens tripartite motif-containing 8 (TRIM8), mRNA | NM_030912 | 13569865 | TRIM8 |
| 2356 | Homo sapiens syncoilin, intermediate filament 1 (SYNC1), mRNA | NM_030786 | 13540560 | SYNC1 |
| 2357 | Homo sapiens BTB and CNC homology 1, basic leucine zipper transcription factor | NM_021813 | 13540489 | BACH2 |
| | 2 (BACH2), mRNA | | | |
| 2358 | Homo sapiens DEAH (Asp-Glu-Ala-His) box polypeptide 9 (DHX9), transcript | NM_001357 | 13514819 | DHX9 |
| | variant 1, mRNA | | | |
| 2359 | Homo sapiens DEAD (Asp-Glu-Ala-Asp) box polypeptide 3, X-linked (DDX3X), | NM_001356 | 13514812 | DDX3X |
| | transcript variant 2, mRNA | | | |
| 2360 | Homo sapiens thioredoxin 1 (TRX1) pseudogene 5 sequence | AF357532 | 13448894 | TRX1 |
| 2361 | Homo sapiens damage-specific DNA binding protein 1, 127 kDa (DDB1), mRNA | NM_001923 | 13435358 | DDB1 |
| 2362 | Homo sapiens ATPase type 13A2 (ATP13A2), mRNA | NM_022089 | 13435128 | ATP13A2 |
| 2363 | Homo sapiens nuclear receptor subfamily 1, group D, member 1 (NR1D1), mRNA | NM_021724 | 13430847 | NR1D1 |
| 2364 | Homo sapiens myc target 1 (MYCT1), mRNA | NM_025107 | 13376676 | MYCT1 |
| 2365 | Homo sapiens coiled-coil domain containing 2 (CCDC2), mRNA | NM_025103 | 13376668 | CCDC2 |
| 2366 | Homo sapiens chromosome 10 open reading frame 88 (C10orf88), mRNA | NM_024942 | 13376420 | C10orf88 |
| 2367 | Homo sapiens hypothetical protein FLJ21908 (FLJ21908), mRNA | NM_024604 | 13375808 | FLJ21908 |
| 2368 | Homo sapiens Janus kinase 2 (a protein tyrosine kinase) (JAK2), mRNA | NM_004972 | 13325062 | JAK2 |
| 2369 | Homo sapiens ARP1 actin-related protein 1 homolog B, centractin beta (yeast) | NM_005735 | 13325060 | ACTR1B |
| | (ACTR1B), mRNA | | | |
| 2370 | Homo sapiens ARP1 actin-related protein 1 homolog A, centractin alpha (yeast) | NM_005736 | 13325058 | ACTR1A |
| | (ACTR1A), mRNA | | | |
| 2371 | Homo sapiens family with sequence similarity 3, member A (FAM3A), mRNA | NM_021806 | 13324681 | FAM3A |
| 2373 | Homo sapiens dynactin 1 (p150, glued homolog, Drosophila) (DCTN1), transcript | NM_004082 | 13259509 | DCTN1 |
| | variant 1, mRNA | | | |
| 2374 | Homo sapiens retinoblastoma binding protein 7 (RBBP7), mRNA | NM_002893 | 13259504 | RBBP7 |
| 2375 | Homo sapiens hypothetical protein MGC2941 (MGC2941), mRNA | NM_024297 | 13236519 | MGC2941 |
| 2376 | Homo sapiens transmembrane protein 38A (TMEM38A), mRNA | NM_024074 | 13129059 | TMEM38A |
| 2377 | Homo sapiens endothelial differentiation, sphingolipid G-protein-coupled receptor, | NM_001400 | 13027635 | EDG1 |
| | 1 (EDG1), mRNA | | | |
| 2378 | Homo sapiens chromosome 20 open reading frame 116 (C20orf116), mRNA | NM_023935 | 13027601 | C20orf116 |
| 2379 | Homo sapiens solute carrier family 39 (zinc transporter), member 6 (SLC39A6), | NM_012319 | 12751474 | SLC39A6 |
| | mRNA | | | |
| 2380 | cand_Human_FGA_30E10_ECM1_long_26Jun2003.fa | NM_004425 | 12707564 | ECM1 |
| | BaseSame: gi\|12707564\|ref\|NM_004425.2\|long variant of ECM1 Homo sapiens | | | |
| | extracellular matrix protein 1 (ECM1), transcript variant 1, mRNA | | | |
| 2381 | Homo sapiens phospholipase D3, mRNA (cDNA clone IMAGE: 3163199), partial | BC000553 | 12653558 | |
| | cds | | | |
| 2382 | cand_Human_STR_101G10_26Jun2003.fa | BC000232 | 12652946 | |
| | BaseSame: gi\|12652946\|gb\|BC000232.1\|gb\|BC000232.1\|Homo sapiens | | | |
| | BC000232 200204, Similar to deleted in polyposis 1, clone MGC: 2267 | | | |
| | IMAGE: 3350749, mRNA, complete cds | | | |
| 2383 | Homo sapiens heparan sulfate 2-O-sulfotransferase 1 (HS2ST1), mRNA | NM_012262 | 12545388 | HS2ST1 |
| 2384 | Homo sapiens chromosome 1 open reading frame 80 (C1orf80), mRNA | NM_022831 | 12383075 | C1orf80 |
| 2385 | cr: gi\|1235671\|gb\|U41766.1\|HSU41766 Human | U41766 | 1235671 | MDC9 |
| | metalloprotease/disintegrin/cysteine-rich protein precursor (MDC9) mRNA, | | | |
| | complete cds | | | |
| 2386 | Homo sapiens major facilitator superfamily domain containing 1 (MFSD1), mRNA | NM_022736 | 12232392 | MFSD1 |
| 2387 | Homo sapiens mRNA; cDNA DKFZp586E1222 (from clone DKFZp586E1222) | AL136930 | 12053354 | |
| 2388 | Homo sapiens DC44 mRNA, complete cds | AF267862 | 12006050 | |
| 2389 | Homo sapiens interferon induced transmembrane protein 3 (1-8U) (IFITM3), | NM_021034 | 11995467 | IFITM3 |
| | mRNA | | | |
| 2390 | Homo sapiens Jumonji, AT rich interactive domain 2 (JARID2), mRNA | NM_004973 | 11863151 | JARID2 |
| 2391 | Homo sapiens ubiquitin specific protease 9, X-linked (fat facets-like, Drosophila) | NM_021906 | 11641422 | USP9X |
| | (USP9X), transcript variant 2, mRNA | | | |
| 2392 | Homo sapiens growth differentiation factor 10 (GDF10), mRNA | NM_004962 | 11641417 | GDF10 |
| 2393 | Homo sapiens glucosidase, alpha; acid (Pompe disease, glycogen storage disease | NM_000152 | 11496988 | GAA |
| | type II) (GAA), mRNA | | | |
| 2394 | Human myosin-IXb mRNA, complete cds | U42391 | 1147782 | |
| 2395 | Homo sapiens prosaposin (variant Gaucher disease and variant metachromatic | NM_002778 | 11386146 | PSAP |
| | leukodystrophy) (PSAP), mRNA | | | |
| 2396 | Homo sapiens myosin, heavy polypeptide 3, skeletal muscle, embryonic (MYH3), | NM_002470 | 11342671 | MYH3 |
| | mRNA | | | |
| 2397 | Homo sapiens matrix metalloproteinase 2 (gelatinase A, 72 kDa gelatinase, 72 kDa | NM_004530 | 11342665 | MMP2 |
| | type IV collagenase) (MMP2), mRNA | | | |
| 2398 | Homo sapiens CD2-associated protein (CD2AP), mRNA | NM_012120 | 11321633 | CD2AP |
| 2399 | Homo sapiens kinase insert domain receptor (a type III receptor tyrosine kinase) | NM_002253 | 11321596 | KDR |
| | (KDR), mRNA | | | |
| 2400 | Homo sapiens succinate-CoA ligase, ADP-forming, beta subunit (SUCLA2), mRNA | NM_003850 | 11321582 | SUCLA2 |
| 2401 | Homo sapiens succinate-CoA ligase, GDP-forming, alpha subunit (SUCLG1), | NM_003849 | 11321580 | SUCLG1 |
| | mRNA | | | |
| 2402 | Homo sapiens myosin, heavy polypeptide 13, skeletal muscle (MYH13), mRNA | NM_003802 | 11321578 | MYH13 |
| 2403 | Homo sapiens myristoylated alanine-rich protein kinase C substrate (MARCKS), | NM_002356 | 11125771 | MARCKS |
| | mRNA | | | |
| 2404 | Homo sapiens tribbles homolog 2 (Drosophila) (TRIB2), mRNA | NM_021643 | 11056053 | TRIB2 |
| 2405 | Homo sapiens serine carboxypeptidase 1 (SCPEP1), mRNA | NM_021626 | 11055991 | SCPEP1 |
| 2406 | Homo sapiens CD79B antigen (immunoglobulin-associated beta) (CD79B), | NM_021602 | 11038675 | CD79B |
| | transcript variant 2, mRNA | | | |
| 2407 | Homo sapiens barrier to autointegration factor 1 (BANF1), mRNA | NM_003860 | 11038645 | BANF1 |
| 2408 | Homo sapiens arginase, liver (ARG1), mRNA | NM_000045 | 10947138 | ARG1 |
| 2409 | Homo sapiens CAP, adenylate cyclase-associated protein 1 (yeast) (CAP1), | NM_006367 | 10938021 | CAP1 |
| | mRNA | | | |
| 2410 | Homo sapiens cyclin B2 (CCNB2), mRNA | NM_004701 | 10938017 | CCNB2 |
| 2411 | Homo sapiens protamine 3 (PRM3), mRNA | NM_021247 | 10864056 | PRM3 |
| 2412 | Homo sapiens pleckstrin homology domain containing, family B (evectins) member | NM_021200 | 10864012 | PLEKHB1 |
| | 1 (PLEKHB1), mRNA | | | |
| 2413 | Homo sapiens thyroid hormone receptor interactor 12 (TRIP12), mRNA | NM_004238 | 10863902 | TRIP12 |
| 2415 | Homo sapiens serpin peptidase inhibitor, clade E (nexin, plasminogen activator | NM_000602 | 10835158 | SERPINE1 |
| | inhibitor type 1), member 1 (SERPINE1), mRNA | | | |
| 2416 | Homo sapiens necdin homolog (mouse) (NDN), mRNA | NM_002487 | 10800414 | NDN |
| 2417 | Human translation initiation factor eIF-2alpha mRNA, 3′UTR | U26032 | 1072033 | |
| 2418 | Homo sapiens cDNA: FLJ23553 fis, clone LNG09301 | AK027206 | 10440278 | |
| 2419 | cr: gi\|10439471\|dbj\|AK026587.1\|Homo sapiens cDNA: FLJ22934 fis, clone | AK026587 | 10439471 | |
| | KAT07572, highly similar to AF100759 Homo sapiens transmembrane 4 | | | |
| | superfamily protein mRNA | | | |
| 2420 | Homo sapiens cDNA: FLJ22657 fis, clone HSI07791, highly similar to HUMCYB5 | AK026310 | 10439131 | |
| | Human cytochrome b5 mRNA | | | |
| 2421 | Homo sapiens cDNA: FLJ22465 fis, clone HRC10233, highly similar to AF161518 | AK026118 | 10438865 | |
| | Homo sapiens HSPC169 mRNA | | | |
| 2422 | Homo sapiens cDNA FLJ13935 fis, clone Y79AA1000800, highly similar to Homo | AK023997 | 10436231 | ZSIG11 |
| | sapiens putative secreted protein (ZSIG11) mRNA | | | |
| 2423 | Homo sapiens cDNA FLJ13700 fis, clone PLACE2000216, highly similar to | AK023762 | 10435792 | |
| | SPECTRIN BETA CHAIN, BRAIN | | | |
| 2424 | Homo sapiens cDNA FLJ13521 fis, clone PLACE1005876, highly similar to | AK023583 | 10435560 | |
| | CLEAVAGE AND POLYADENYLATION SPECIFICITY FACTOR, 100 KD | | | |
| | SUBUNIT | | | |
| 2426 | Homo sapiens growth differentiation factor 3 (GDF3), mRNA | NM_020634 | 10190669 | GDF3 |
| 2427 | Homo sapiens heat shock 70 kDa protein 14 (HSPA14), mRNA | NM_016299 | 10047093 | HSPA14 |
| 2428 | Homo sapiens FXYD domain containing ion transport regulator 5 (FXYD5), | NM_144779.1 | 47778937 | FXYD5 |
| | transcript variant 1, mRNA | | | |

### Example 3

### Testing the siRNA Compounds in Primary Newborn Rat Cardiomyocytes (NRVM)

Primary neonatal rat cardiomyocytes (NRVM cells) were infected with shRNA expression vector (pTZ-GFP-H1RNA) in which the specific siRNA sequences had been inserted. 72 hrs after infection, cells were starved for 24 hrs and stretching was induced for 2 hrs. Real-Time PCR for the genes and their specific markers was performed to determine the extent of the endogenous gene expression inhibition.

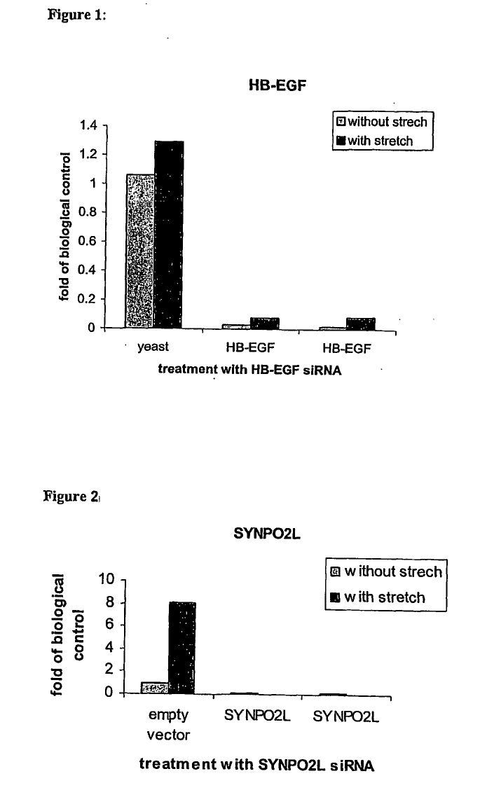

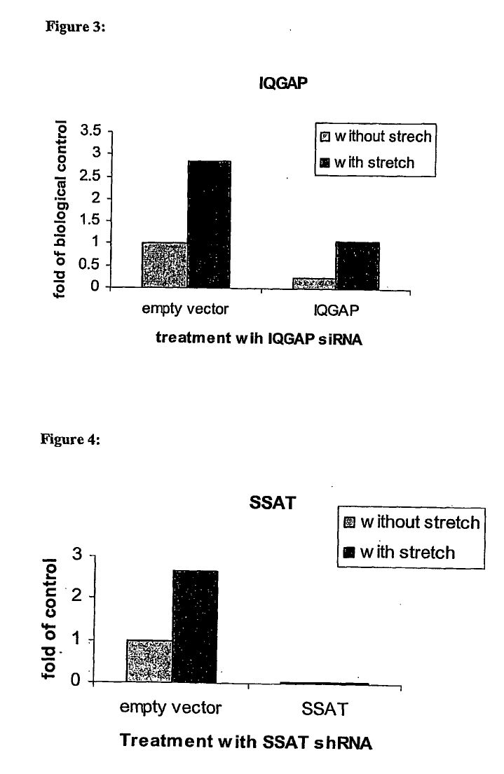

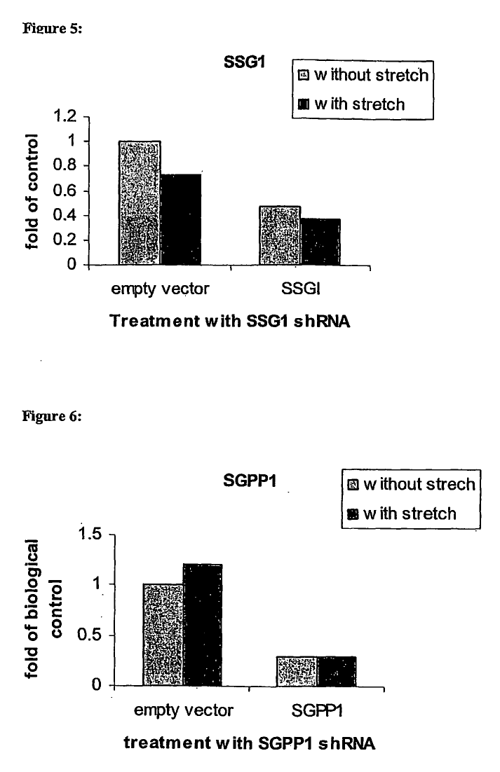

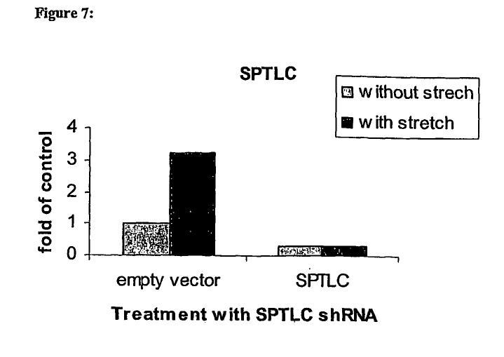

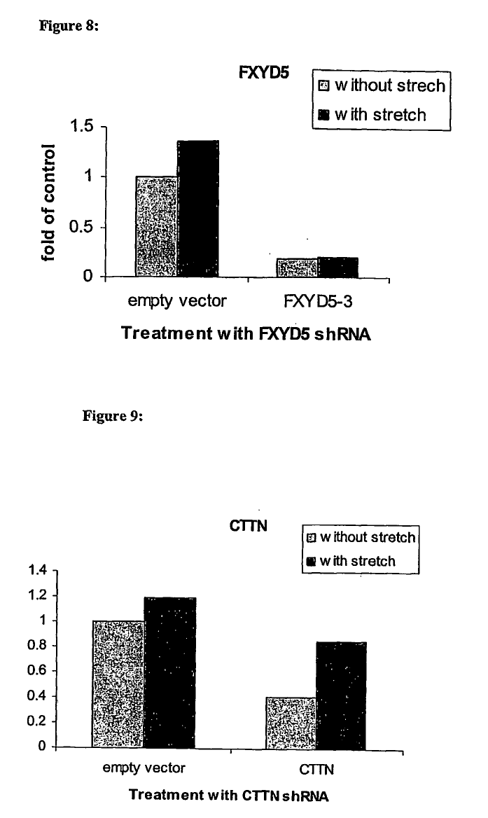

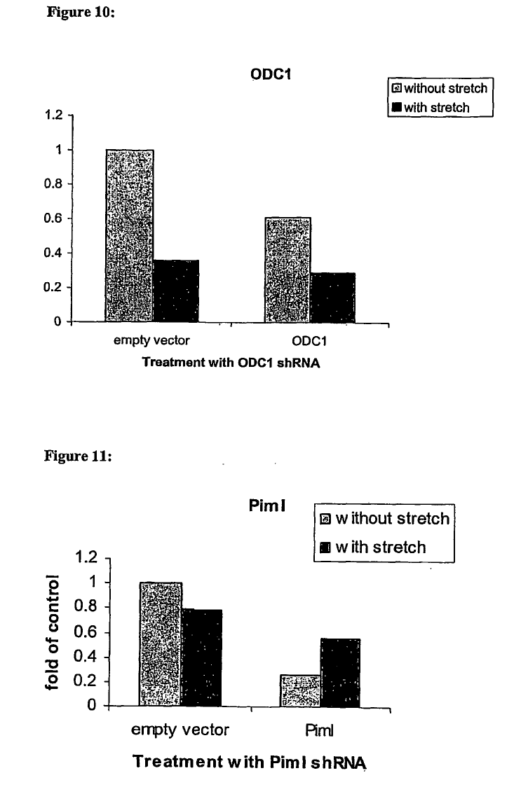

The NRVM cells were infected using the Lentiviral infection method with specific shRNA expressing various siRNA compounds corresponding to the following genes: SYNPO2L, HB-EGF, IQGAP, SSAT1, SSG1, SGPP1, SPTLC2, FXYD5, CTTN, ODC1 and PIM1 (Table B). The expression of the specific endogenous gene was evaluated with or without stretching treatment. As revealed from FIGS. 1-11, the expression of the endogenous selected genes exhibited a significant increase following the induction of stretching in the cells as determined by Real-Time PCR analysis. Infection with the specific shRNA significantly inhibited the expression of the selected endogenous genes before and following stretching.

### Example 4

### In Vivo Experimental Models

Myocardial infarction is inflicted by permanent ligation of the left coronary artery (LCA) as described by Lutgens et al. in Cardiovasc. Res. (1999) 41:586-59. Briefly, mice are anesthetized by intraperitoneal injection of 60 mg/kg sodium pentobarbital. A transverse skin incision above the third intercostal space and a left thoracotomy between the third and fourth ribs are made, and a 6.0 filament are tied around the LCA about 1 mm distal from the tip of the left auricle. After closure of the chest cavity and re-expansion of the lungs using positive pressure at end expiration, the infarcted mice are allowed to recover on a warming pad.

An osmotic minipump delivering the compounds of the invention (e.g. oligonucleotides, vectors or antibodies) or a placebo during the experiment is implanted subcutaneously on the back of the mouse immediately after performance of myocardial infarction. At 4 and 24 hours and at time points up to 16 days after surgery, infarcted mice are anesthetized and perfused with 1% paraformaldehyde in 0.1 M phosphate buffered saline (pH 7.0) via the abdominal aorta at physiological pressure. Fixed hearts are dissected and prepared for histology. 6 μm-thin sections are used for haematoxylin-eosin staining and for staining with markers specific for myocardial infarction to determine the effect of the treatment by the compounds of the invention on the mice as compared to control mice which receive a placebo or which were untreated (no myocardial infarction).

### Example 5

### In Situ Hybridization Analysis Following Left Anterior Descending Coronary Artery (LAD) Ligation

C3HeB/FeJ mice underwent ligation of left anterior descending (LAD) coronary artery and the expression of selected genes in the myocardium was analyzed by In situ hybridization (ISH) at various time points (0, 4 hr, 24 h and 4, 8, 16 and 24 days post ligation). Table P below details the ISH results obtained with representative genes following the LAD ligation. As revealed from this table, SYNPO2, IQ-GAP, FXYD5, HB-EGF, PIM1, SPTLC2, SSAT1 and SSG1 exhibit specific up-regulation of expression following LAD ligation and the consequent myocardial injury.

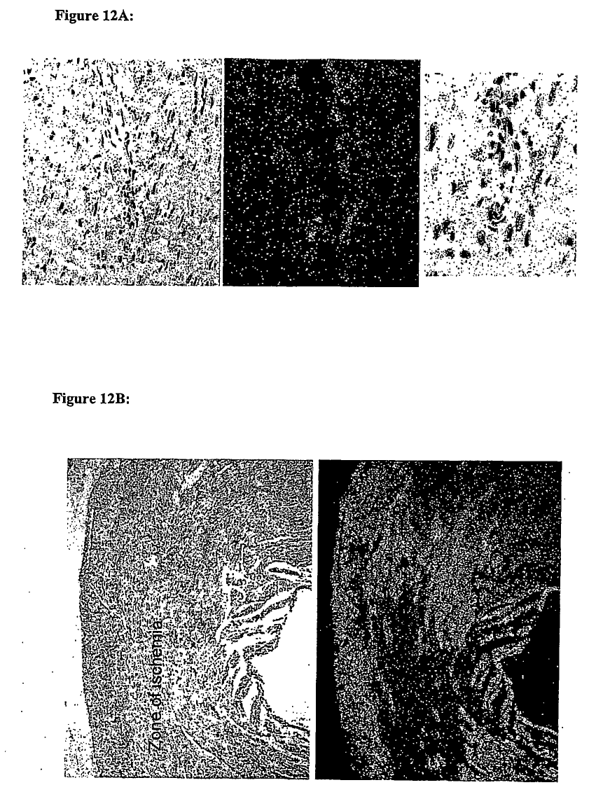

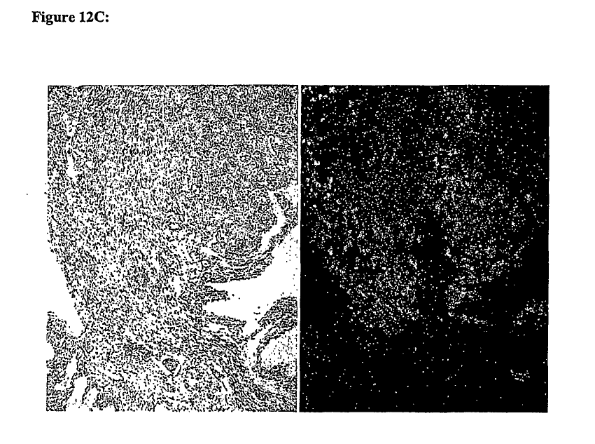

FIG. 12A specifically demonstrates Synpo2L expression (using ISH) in perivascular cardiomyocytes of intact myocardium. Middle panel is ISH analysis using fluorescence staining. The expression in the periinfarct area at 24 hrs post-ligation is illustrated in FIG. 12B (Right panel). The expression in the periinfarct area at 24 days post-ligation is illustrated in FIG. 12C (Right panel). These results show that the expression of Synpo2L is up-regulated following myocardial injury.

TABLE P

| Gene | ISH analysis post-ligation |
|---|---|
| Synaptopodin 2-like | Constitutive expression in perivascular |
| (SYNPO2) | cardiomyocytes. |
| | Activated expression at 4 hr-24 hr in |
| | multiple cardiomyocytes. Transient |
| | expression in endocardial and endothelial |
| | cells. |
| IQ motif containing GTPase | No expression in intact myocardium. |
| activating protein 1 (IQ-GAP) | Activated expression in inflammatory |
| | and fibroblast-like cells at 24 hr-8 d. |
| FXYD domain containing ion | No expression in intact myocardium. |
| transport regulator 5 (FXYD5) | Expression activated in perivascular, |
| | interstitial, endocardial and pericardial |
| | cells at 24 hr post-ligation. |
| | Prominent expression in |
| | granulation/fibrotic tissue at 4-16 days |
| | post-ligation. |
| heparin-binding EGF-like | No expression in intact myocardium. |
| growth factor (HB-EGF | Multiple cardiomyocytes show rapid |
| (DTR)b) | expression at 4 hr and 24 hr post-ligation |
| | at the periphery of ischemic zone. |
| | Subset of cardiomyocytes show |
| | expression at later stages of tissue |
| | remodeling in response to ischemic |
| | injury. |
| pim-1 oncogene (PIM1) | No expression in intact myocardium. |
| | Expression activated at 4 hr in subset of |
| | cardiomyocytes and perivascular cells at |
| | the periphery of ischemic area. |
| | Down regulation of expression by 24 h. |
| serine palmitoyltransferase, | No expression in intact myocardium. |
| long chain base subunit 2 | Widely expressed in fibroblasts and |
| (SPTLC2) | inflammatory cells at early stage of post- |
| | infarct tissue repair. |
| spermidine/spermine N1- | No expression in intact myocardium. |
| acetyltransferase (SSAT1) | Rapid activation of expression at 4 hr and |
| | 24 hr post-ligation in non-contractile |
| | elements of myocardium (perivascular |
| | cells, interstitial fibroblast-like cells, |
| | pericardial and endocardial cells) in |
| | response to injury. |
| steroid sensitive gene 1 | Expression in perivascular and fibroblast- |
| (URB), | like cells of intact myocardium. |
| transcript variant 1 (SSG1) | Development of fibrotic scar is |
| | accompanied by accumulation of |
| | multiple expressing fibroblast-like cells |
| | at all stages of tissue remodeling. |

1. A compound having the structure:
5′ (N)ₓ-Z 3′ (antisense strand)
3′Z′-(N′)_{y} 5′ (sense strand)
wherein each N and N′ is a ribonucleotide which may be modified or unmodified in its sugar residue and (N)ₓ and (N′)_{y} is an oligomer in which each consecutive N or N′ is joined to the next N or N′ by a covalent bond;
wherein each of x and y is an integer between 19 and 40;
wherein each of Z and Z′ may be present or absent, but if present is dTdT and is covalently attached at the 3′ terminus of the strand in which it is present;
and wherein the sequence of (N)ₓ comprises an antisense sequence set forth in any of SEQ ID NOS: 40-78.

2. (canceled)

3. The compound of claim 1, wherein the covalent bond is a phosphodiester bond.

4. The compound of claim 1, wherein x=y=19.

5. The compound of claim 1, wherein Z and Z′ are both absent.

6. The compound of claim 1, wherein one of Z or Z′ is present.

7. The compound of claim 1, wherein all of the ribonucleotides are unmodified in their sugar residues.

8. The compound of claim 1, wherein at least one ribonucleotide is modified in its sugar residue.

9. The compound of claim 8, wherein the modification of the sugar residue comprises a modification at the 2′ position.

10. The compound of claim 9, wherein the modification at the 2′ position results in the presence of a moiety selected from the group consisting of amino, fluoro, alkoxy and alkyl groups.

11. The compound of claim 10, wherein the moiety at the 2′ position is a methoxy (2′-0-methyl) group.

12. The compound of claim 11, wherein alternating ribonucleotides are modified in both the antisense and the sense strands.

13. The compound of claim 12, wherein the ribonucleotides at the 5′ and 3′ termini of the antisense strand are modified in their sugar residues, and the ribonucleotides at the 5′ and 3′ termini of the sense strand are unmodified in their sugar residues.

14. The compound of claim 13, wherein the antisense and the sense strands are non-phosphorylated at the 3′ and 5′ termini or wherein the antisense and the sense strands are phosphorylated at the 3′ termini.

15. A vector capable of expressing a compound having the structure:
5′ (N)ₓ-Z 3′ (antisense strand)
3′ Z′-(N′)_{y} 5′ (sense strand)
wherein each N and N′ is a ribonucleotide which may be modified or unmodified in its sugar residue and (N)ₓ and (N′)_{y} is an oligomer in which each consecutive N or N′ is joined to the next N or N′ by a covalent bond;
wherein each of x and y is an integer between 19 and 40;
wherein each of Z and Z′ may be present or absent, but if present is dTdT and is covalently attached at the 3′ terminus of the strand in which it is present;
and wherein the sequence of (N)ₓ comprises an antisense sequence set forth in any of SEQ ID NOS: 40-78.

16. A pharmaceutical composition comprising a compound of claim 1 in an amount effective to inhibit expression of a gene and a pharmaceutically acceptable carrier.

17. (canceled)

18. A method of treating a patient suffering from a cardiovascular disorder comprising administering to the patient a pharmaceutical composition according to claim 16 in a therapeutically effective dose so as to thereby treat the patient.

19-26. (canceled)

27. The method according to claim 18 wherein the cardiovascular disorder is selected from: myocardial ischemia-associated dysfunction, coronary arteriosclerosis, coronary thrombosis, myocardial infarction, stroke, acute coronary syndrome, unstable angina, arrhythmia, cardiopulmonary arrest, valve disorders and cardiac chest pain.

28-30. (canceled)

31. A method of down-regulating the expression of a cardiovascular-related gene by at least 50% as compared to a control comprising contacting an mRNA transcript of the gene with the compound of claim 1.

32. (canceled)

33. A pharmaceutical composition comprising a compound of claim 13 in an amount effective to inhibit expression of a gene and a pharmaceutically acceptable carrier.
